SEQUENCE LISTING (1249 sequences)

SEQ ID NO: 1 - protein, 415 aa, Homo sapiens
YNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAEAVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLRSTKFTIYNNMFCAGFHEGGRDSCQGDSGGPHVTEVEGTSFLTGIISWGEECAMKGKYGIYTKVSRYVNWIKEKTKLT

SEQ ID NO: 2 - protein, 448 aa, Homo sapiens [sequence omitted]

SEQ ID NO: 3 - protein, 122 aa, Artificial Sequence
  Recombinant protein
EVQLVESGGGLVQPGRSLKLSCAASGFTFDDYAMHWVRQVPGKGLEWVSGISWRGDIGGYVDSVKGRFTISRDNAKNSLYLQLNSLRAEDTALYYCVKSYGSGSFYNAFDSWGQGTLVTVSS

SEQ ID NO: 4 - protein, 5 aa, Artificial Sequence
  Recombinant protein
DYAMH

SEQ ID NO: 5 - protein, 17 aa, Artificial Sequence
  Recombinant protein
GISWRGDIGGYVDSVKG

SEQ ID NO: 6 - protein, 13 aa, Artificial Sequence
  Recombinant protein
SYGSGSFYNAFDS

SEQ ID NO: 7 - protein, 107 aa, Artificial Sequence
  Recombinant protein
DIQMTQSPSTLSASAGDRVTITCRASQSISSWLAWYQQKPGKAPKFLIYKASRLERGVPSRFSGSGSGTEFSLTISSLQPDDFATYYCLEYSSYIRTFGQGTKVEIK

SEQ ID NO: 8 - protein, 11 aa, Artificial Sequence
  Recombinant protein
RASQSISSWLA

SEQ ID NO: 9 - protein, 7 aa, Artificial Sequence
  Recombinant protein
KASRLER

SEQ ID NO: 10 - protein, 9 aa, Artificial Sequence
  Recombinant protein
LEYSSYIRT

SEQ ID NO: 11 - protein, 122 aa, Artificial Sequence
  Recombinant protein
EVQLVESGGGLVQPGRSLKLSCAASGFTFHDYAMHWVRQVPGKGLEWVSGISWRGDIIGYVDSVKGRFTISRDNAKNSLYLQLNSLRAEDTALYYCVKSYGSGSFYNAFDSWGQGTLVTVSS

SEQ ID NO: 12 - protein, 5 aa, Artificial Sequence
  Recombinant protein
DYAMH

SEQ ID NO: 13 - protein, 17 aa, Artificial Sequence
  Recombinant protein
GISWRGDIIGYVDSVKG

SEQ ID NO: 14 - protein, 13 aa, Artificial Sequence
  Recombinant protein
SYGSGSFYNAFDS

SEQ ID NO: 15 - protein, 107 aa, Artificial Sequence
  Recombinant protein
DIQMTQSPSTLSASAGDEVTITCRASKSISSWLAWYQQKPGKAPRFLIYKASRLDRGTPSRFSGSGSGTEFTLTISHLQPDDFATYYCLEYSSYIRTFGQGTKVEIK

SEQ ID NO: 16 - protein, 11 aa, Artificial Sequence
  Recombinant protein
RASKSISSWLA

SEQ ID NO: 17 - protein, 7 aa, Artificial Sequence
  Recombinant protein
KASRLDR

SEQ ID NO: 18 - protein, 9 aa, Artificial Sequence
  Recombinant protein
LEYSSYIRT

SEQ ID NO: 19 - protein, 122 aa, Artificial Sequence; identical to SEQ ID NO: 3
  Recombinant protein

SEQ ID NO: 20 - protein, 5 aa, Artificial Sequence
  Recombinant protein
DYAMH

SEQ ID NO: 21 - protein, 17 aa, Artificial Sequence
  Recombinant protein
GISWRGDIGGYVDSVKG

SEQ ID NO: 22 - protein, 13 aa, Artificial Sequence
  Recombinant protein
SYGSGSFYNAFDS

SEQ ID NO: 23 - protein, 107 aa, Artificial Sequence
  Recombinant protein
DIQMTQSPSTLSASAGDRVTITCRASQSISSWLAWYQQKPGKAPKFLIYKASRLDRGTPSRFSGSGSGTEFSLTISSLQPDDFATYYCLEYSSYIRTFGQGTKVEIK

SEQ ID NO: 24 - protein, 11 aa, Artificial Sequence
  Recombinant protein
RASQSISSWLA

SEQ ID NO: 25 - protein, 7 aa, Artificial Sequence
  Recombinant protein
KASRLDR

SEQ ID NO: 26 - protein, 9 aa, Artificial Sequence
  Recombinant protein
LEYSSYIRT

SEQ ID NO: 27 - protein, 122 aa, Artificial Sequence; identical to SEQ ID NO: 3
  Recombinant protein

SEQ ID NO: 28 - protein, 5 aa, Artificial Sequence
  Recombinant protein
DYAMH

SEQ ID NO: 29 - protein, 17 aa, Artificial Sequence
  Recombinant protein
GISWRGDIGGYVDSVKG

SEQ ID NO: 30 - protein, 13 aa, Artificial Sequence
  Recombinant protein
SYGSGSFYNAFDS

SEQ ID NO: 31 - protein, 107 aa, Artificial Sequence
  Recombinant protein
DIQMTQSPSTLSASVGDRVTITCRASQSISSWLAWYQQKPGKAPKFLIYKASRLERGTPSRFSGSGSGTEFSLTISSLQPDDFATYYCLEYSSYIRTFGQGTKVEIK

SEQ ID NO: 32 - protein, 11 aa, Artificial Sequence
  Recombinant protein
RASQSISSWLA

SEQ ID NO: 33 - protein, 7 aa, Artificial Sequence
  Recombinant protein
KASRLER

SEQ ID NO: 34 - protein, 9 aa, Artificial Sequence
  Recombinant protein
LEYSSYIRT

SEQ ID NO: 35 - protein, 122 aa, Artificial Sequence
  Recombinant protein
EVQLVESGGGLVQPGRSLKLSCAASGFTFDDYAMHWVRQVPGKGLEWVSGISWKGDIGGYVDSVKGRFTISRDNAKNSLYLQLNSLRAEDTALYYCAKSYGSGSFYNAFDSWGQGTLVTVSS

SEQ ID NO: 36 - protein, 5 aa, Artificial Sequence
  Recombinant protein
DYAMH

SEQ ID NO: 37 - protein, 17 aa, Artificial Sequence
  Recombinant protein
GISWKGDIGGYVDSVKG

SEQ ID NO: 38 - protein, 13 aa, Artificial Sequence
  Recombinant protein
SYGSGSFYNAFDS

SEQ ID NO: 39 - protein, 107 aa, Artificial Sequence
  Recombinant protein
DIQMTQSPSTLSASAGDEVTITCRASQSISSWLAWYQQKPGKAPKLLIYKASKLDRGVPSRFSGSGSGTEFSLTISDLQPDDFATYYCLEYSSYIRTFGQGTKVEIK

SEQ ID NO: 40 - protein, 11 aa, Artificial Sequence
  Recombinant protein
RASQSISSWLA

SEQ ID NO: 41 - protein, 7 aa, Artificial Sequence
  Recombinant protein
KASKLDR

SEQ ID NO: 42 - protein, 9 aa, Artificial Sequence
  Recombinant protein
LEYSSYIRT

SEQ ID NO: 43 - protein, 122 aa, Artificial Sequence
  Recombinant protein
EVQLVESGGGLVQPGRSLRLSCAASGFTFHDYAMHWVRQVPGKGLEWVSGISWKGDIGGYVDSVKGRFTISRDNEKNSLYLQMNSLRAEDTALYYCVKSYGSGSFYNAFDSWGQGTLVTVSS

SEQ ID NO: 44 - protein, 5 aa, Artificial Sequence
  Recombinant protein
DYAMH

SEQ ID NO: 45 - protein, 17 aa, Artificial Sequence
  Recombinant protein
GISWKGDIGGYVDSVKG

SEQ ID NO: 46 - protein, 13 aa, Artificial Sequence
  Recombinant protein
SYGSGSFYNAFDS

SEQ ID NO: 47 - protein, 107 aa, Artificial Sequence
  Recombinant protein
DIQMTQSPSTLSASVGDRVTITCRASQSISSWLAWYQQKPGKAPKLLIYKASRLERGTPSRFSGSGSGTEFSLTISSLQPDDFATYYCLEYSSYIRTFGQGTKVEIK

SEQ ID NO: 48 - protein, 11 aa, Artificial Sequence
  Recombinant protein
RASQSISSWLA

SEQ ID NO: 49 - protein, 7 aa, Artificial Sequence
  Recombinant protein
KASRLER

SEQ ID NO: 50 - protein, 9 aa, Artificial Sequence
  Recombinant protein
LEYSSYIRT

SEQ ID NO: 51 - protein, 122 aa, Artificial Sequence
  Recombinant protein
EVQLVESGGGLVQPGRSLRLSCAASGFTFHDYAMHWVRQVPGKGLEWVSGISWRGDIGGYVKSVKGRFTISRDNAKNSLYLQMNSLRAEDTALYYCVKSYGSGSFYNAFDSWGQGTLVTVSS

SEQ ID NO: 52 - protein, 5 aa, Artificial Sequence
  Recombinant protein
DYAMH

SEQ ID NO: 53 - protein, 17 aa, Artificial Sequence
  Recombinant protein
GISWRGDIGGYVKSVKG

SEQ ID NO: 54 - protein, 13 aa, Artificial Sequence
  Recombinant protein
SYGSGSFYNAFDS

SEQ ID NO: 55 - protein, 107 aa, Artificial Sequence
  Recombinant protein
DIQMTQSPSTLSASVGDRVTITCRASQSISSWLAWYQQKPGKAPKFLIYKASKLERGTPSRFSGSGSGTEFSLTISSLQPDDFATYYCLEYSSYIRTFGQGTKVEIK

SEQ ID NO: 56 - protein, 11 aa, Artificial Sequence
  Recombinant protein
RASQSISSWLA

SEQ ID NO: 57 - protein, 7 aa, Artificial Sequence
  Recombinant protein
KASKLER

SEQ ID NO: 58 - protein, 9 aa, Artificial Sequence
  Recombinant protein
LEYSSYIRT

SEQ ID NO: 59 - protein, 122 aa, Artificial Sequence; identical to SEQ ID NO: 51
  Recombinant protein

SEQ ID NO: 60 - protein, 5 aa, Artificial Sequence
  Recombinant protein
DYAMH

SEQ ID NO: 61 - protein, 17 aa, Artificial Sequence
  Recombinant protein
GISWRGDIGGYVKSVKG

SEQ ID NO: 62 - protein, 13 aa, Artificial Sequence
  Recombinant protein
SYGSGSFYNAFDS

SEQ ID NO: 63 - protein, 107 aa, Artificial Sequence; identical to SEQ ID NO: 31
  Recombinant protein

SEQ ID NO: 64 - protein, 11 aa, Artificial Sequence
  Recombinant protein
RASQSISSWLA

SEQ ID NO: 65 - protein, 7 aa, Artificial Sequence
  Recombinant protein
KASRLER

SEQ ID NO: 66 - protein, 9 aa, Artificial Sequence
  Recombinant protein
LEYSSYIRT

SEQ ID NO: 67 - protein, 122 aa, Artificial Sequence; identical to SEQ ID NO: 43
  Recombinant protein

SEQ ID NO: 68 - protein, 5 aa, Artificial Sequence
  Recombinant protein
DYAMH

SEQ ID NO: 69 - protein, 17 aa, Artificial Sequence
  Recombinant protein
GISWKGDIGGYVDSVKG

SEQ ID NO: 70 - protein, 13 aa, Artificial Sequence
  Recombinant protein
SYGSGSFYNAFDS

SEQ ID NO: 71 - protein, 107 aa, Artificial Sequence
  Recombinant protein
DIQMTQSPSTLSASVGDRVTITCRASQSISSWLAWYQQKPGKAPKFLIYKASRLDRGTPSRFSGSGSGTEFSLTISSLQPDDFATYYCLEYSSYIRTFGQGTKVEIK

SEQ ID NO: 72 - protein, 11 aa, Artificial Sequence
  Recombinant protein
RASQSISSWLA

SEQ ID NO: 73 - protein, 7 aa, Artificial Sequence
  Recombinant protein
KASRLDR

SEQ ID NO: 74 - protein, 9 aa, Artificial Sequence
  Recombinant protein
LEYSSYIRT

SEQ ID NO: 75 - protein, 122 aa, Artificial Sequence
  Recombinant protein
EVQLVESGGGLVQPGRSLRLSCAASGFTFHDYAMHWVRQAPGKGLEWVSGISWRGDIKGYVDSVKGRFTISRDNAKNSLYLQLNSLRAEDTALYYCAKSYGSGSFYNAFDSWGQGTLVTVSS

SEQ ID NO: 76 - protein, 5 aa, Artificial Sequence
  Recombinant protein
DYAMH

SEQ ID NO: 77 - protein, 17 aa, Artificial Sequence
  Recombinant protein
GISWRGDIKGYVDSVKG

SEQ ID NO: 78 - protein, 13 aa, Artificial Sequence
  Recombinant protein
SYGSGSFYNAFDS

SEQ ID NO: 79 - protein, 107 aa, Artificial Sequence
  Recombinant protein
DIQMTQSPSTLSASAGDRVTITCRASKSISSWLAWYQQKPGKAPKFLIYKASRLDRGTPQRFSGSGSGTEFSLTISSLQPDDFATYYCLEYSSYIRTFGQGTKVEIK

SEQ ID NO: 80 - protein, 11 aa, Artificial Sequence
  Recombinant protein
RASKSISSWLA

SEQ ID NO: 81 - protein, 7 aa, Artificial Sequence
  Recombinant protein
KASRLDR

SEQ ID NO: 82 - protein, 9 aa, Artificial Sequence
  Recombinant protein
LEYSSYIRT

SEQ ID NO: 83 - protein, 122 aa, Artificial Sequence; identical to SEQ ID NO: 75
  Recombinant protein

SEQ ID NO: 84 - protein, 5 aa, Artificial Sequence
  Recombinant protein
DYAMH

SEQ ID NO: 85 - protein, 17 aa, Artificial Sequence
  Recombinant protein
GISWRGDIKGYVDSVKG

SEQ ID NO: 86 - protein, 13 aa, Artificial Sequence
  Recombinant protein
SYGSGSFYNAFDS

SEQ ID NO: 87 - protein, 107 aa, Artificial Sequence
  Recombinant protein
DIQMTQSPSTLSASAGDRVTISCRASQSISSWLAWYQQKPGKAPKLLIYKASRLDRGTPSRFSGSGSGTEFTLTISSLQPDDFATYYCLEYNSYIRTFGQGTKVEIK

SEQ ID NO: 88 - protein, 11 aa, Artificial Sequence
  Recombinant protein
RASQSISSWLA

SEQ ID NO: 89 - protein, 7 aa, Artificial Sequence
  Recombinant protein
KASRLDR

SEQ ID NO: 90 - protein, 9 aa, Artificial Sequence
  Recombinant protein
LEYNSYIRT

SEQ ID NO: 91 - protein, 122 aa, Artificial Sequence
  Recombinant protein
EVQLVESGGGLVQPGRSLKLSCAASGFTFHDYAMHWVRQVPGKGLEWVSGISWRGDIGGYVKSVKGRFTISRDNAKNSLYLQLNSLRAEDTALYYCVKSYGSGSFYNAFDSWGQGTLVTVSS

SEQ ID NO: 92 - protein, 5 aa, Artificial Sequence
  Recombinant protein
DYAMH

SEQ ID NO: 93 - protein, 17 aa, Artificial Sequence
  Recombinant protein
GISWRGDIGGYVKSVKG

SEQ ID NO: 94 - protein, 13 aa, Artificial Sequence
  Recombinant protein
SYGSGSFYNAFDS

SEQ ID NO: 95 - protein, 107 aa, Artificial Sequence
  Recombinant protein
DIQMTQSPSTLSASVGDEVTITCRASQSISSWLAWYQQKPGKAPKFLIYKAQRLDRGTPQRFSGSGSGTEFTLTISSLQPDDFATYYCLEYSSYIRTFGQGTKVEIK

SEQ ID NO: 96 - protein, 11 aa, Artificial Sequence
  Recombinant protein
RASQSISSWLA

SEQ ID NO: 97 - protein, 7 aa, Artificial Sequence
  Recombinant protein
KAQRLDR

SEQ ID NO: 98 - protein, 9 aa, Artificial Sequence
  Recombinant protein
LEYSSYIRT

SEQ ID NO: 99 - protein, 122 aa, Artificial Sequence
  Recombinant protein
EVQLVESGGGLVQPGRSLKLSCAASGFTFDDYAMHWVRQAPGKGLEWVSGISWKGDIGGYVDSVKGRFTISRDNAKNSLYLQMNSLRAEDTALYYCVKSYGSGSFYNAFDSWGQGTLVTVSS

SEQ ID NO: 100 - protein, 5 aa, Artificial Sequence
  Recombinant protein
DYAMH

SEQ ID NO: 101 - protein, 17 aa, Artificial Sequence
  Recombinant protein
GISWKGDIGGYVDSVKG

SEQ ID NO: 102 - protein, 13 aa, Artificial Sequence
  Recombinant protein
SYGSGSFYNAFDS

SEQ ID NO: 103 - protein, 107 aa, Artificial Sequence
  Recombinant protein
DIQMTQSPSTLSASAGDEVTITCRASQSIKSWLAWYQQKPGKAPKFLIYKASRLDRGTPQRFSGSGSGTEFSLTISRLQPDDFATYYCLEYSSYIRTFGQGTKVEIK

SEQ ID NO: 104 - protein, 11 aa, Artificial Sequence
  Recombinant protein
RASQSIKSWLA

SEQ ID NO: 105 - protein, 7 aa, Artificial Sequence
  Recombinant protein
KASRLDR

SEQ ID NO: 106 - protein, 9 aa, Artificial Sequence
  Recombinant protein
LEYSSYIRT

SEQ ID NO: 107 - protein, 122 aa, Artificial Sequence
  Recombinant protein
EVQLVESGGGLVQPGRSLKLSCAASGFRFDDYAMHWVRQVPGKGLEWVSGISWKGDIGGYADSVKGRFTISRDNAKNSLYLQLNSLRAEDTALYYCVKSYGSGSFYNAFDSWGQGTLVTVSS

SEQ ID NO: 108 - protein, 5 aa, Artificial Sequence
  Recombinant protein
DYAMH

SEQ ID NO: 109 - protein, 17 aa, Artificial Sequence
  Recombinant protein
GISWKGDIGGYADSVKG

SEQ ID NO: 110 - protein, 13 aa, Artificial Sequence
  Recombinant protein
SYGSGSFYNAFDS

SEQ ID NO: 111 - protein, 107 aa, Artificial Sequence
  Recombinant protein
DIQMTQSPSTLSASVGDEVTITCRASKSISSWLAWYQQKPGKAPRFLIYKASRLERGTPQRFSGSGSGTEFTLTIYSLQPDDFATYYCLEYSSYIRTFGQGTKVEIK

SEQ ID NO: 112 - protein, 11 aa, Artificial Sequence
  Recombinant protein
RASKSISSWLA

SEQ ID NO: 113 - protein, 7 aa, Artificial Sequence
  Recombinant protein
KASRLER

SEQ ID NO: 114 - protein, 9 aa, Artificial Sequence
  Recombinant protein
LEYSSYIRT

SEQ ID NO: 115 - protein, 122 aa, Artificial Sequence; identical to SEQ ID NO: 107
  Recombinant protein

SEQ ID NO: 116 - protein, 5 aa, Artificial Sequence
  Recombinant protein
DYAMH

SEQ ID NO: 117 - protein, 17 aa, Artificial Sequence
  Recombinant protein
GISWKGDIGGYADSVKG

SEQ ID NO: 118 - protein, 13 aa, Artificial Sequence
  Recombinant protein
SYGSGSFYNAFDS

SEQ ID NO: 119 - protein, 107 aa, Artificial Sequence; identical to SEQ ID NO: 31
  Recombinant protein

SEQ ID NO: 120 - protein, 11 aa, Artificial Sequence
  Recombinant protein
RASQSISSWLA

SEQ ID NO: 121 - protein, 7 aa, Artificial Sequence
  Recombinant protein
KASRLER

SEQ ID NO: 122 - protein, 9 aa, Artificial Sequence
  Recombinant protein
LEYSSYIRT

SEQ ID NO: 123 - protein, 122 aa, Artificial Sequence
  Recombinant protein
EVQLVESGGGLVQPGRSLKLSCAASGFTFHDYAMHWVRQAPGKGLEWVSGISWKGDIGGYVDSVKGRFTISRDNAKNSLYLQMNSLRAEDTALYYCAKSYGSGSFYNAFDSWGQGTLVTVSS

SEQ ID NO: 124 - protein, 5 aa, Artificial Sequence
  Recombinant protein
DYAMH

SEQ ID NO: 125 - protein, 17 aa, Artificial Sequence
  Recombinant protein
GISWKGDIGGYVDSVKG

SEQ ID NO: 126 - protein, 13 aa, Artificial Sequence
  Recombinant protein
SYGSGSFYNAFDS

SEQ ID NO: 127 - protein, 107 aa, Artificial Sequence
  Recombinant protein
DIQMTQSPSTLSASVGDRVTITCRASQSISSWLAWYQQKPGKAPKFLIYKASKLDRGTPSRFSGSGSGTEFTLTISSLQPDDFATYYCLEYSSYIRTFGQGTKVEIK

SEQ ID NO: 128 - protein, 11 aa, Artificial Sequence
  Recombinant protein
RASQSISSWLA

SEQ ID NO: 129 - protein, 7 aa, Artificial Sequence
  Recombinant protein
KASKLDR

SEQ ID NO: 130 - protein, 9 aa, Artificial Sequence
  Recombinant protein
LEYSSYIRT

SEQ ID NO: 131 - protein, 122 aa, Artificial Sequence; identical to SEQ ID NO: 35
  Recombinant protein

SEQ ID NO: 132 - protein, 5 aa, Artificial Sequence
  Recombinant protein
DYAMH

SEQ ID NO: 133 - protein, 17 aa, Artificial Sequence
  Recombinant protein
GISWKGDIGGYVDSVKG

SEQ ID NO: 134 - protein, 13 aa, Artificial Sequence
  Recombinant protein
SYGSGSFYNAFDS

SEQ ID NO: 135 - protein, 107 aa, Artificial Sequence; identical to SEQ ID NO: 23
  Recombinant protein

SEQ ID NO: 136 - protein, 11 aa, Artificial Sequence
  Recombinant protein
RASQSISSWLA

SEQ ID NO: 137 - protein, 7 aa, Artificial Sequence
  Recombinant protein
KASRLDR

SEQ ID NO: 138 - protein, 9 aa, Artificial Sequence
  Recombinant protein
LEYSSYIRT

SEQ ID NO: 139 - protein, 122 aa, Artificial Sequence; identical to SEQ ID NO: 43
  Recombinant protein

SEQ ID NO: 140 - protein, 5 aa, Artificial Sequence
  Recombinant protein
DYAMH

SEQ ID NO: 141 - protein, 17 aa, Artificial Sequence
  Recombinant protein
GISWKGDIGGYVDSVKG

SEQ ID NO: 142 - protein, 13 aa, Artificial Sequence
  Recombinant protein
SYGSGSFYNAFDS

SEQ ID NO: 143 - protein, 107 aa, Artificial Sequence
  Recombinant protein
DIQMTQSPSTLSASVGDRVTITCRASQSISSWLAWYQQKPGKAPKFLIYKASRLDRGCPSRFSGSGSGTEFSLTISSLQPDDFATYYCLEYSSYIRTFGQGTKVEIK

SEQ ID NO: 144 - protein, 11 aa, Artificial Sequence
  Recombinant protein
RASQSISSWLA

SEQ ID NO: 145 - protein, 7 aa, Artificial Sequence
  Recombinant protein
KASRLDR

SEQ ID NO: 146 - protein, 9 aa, Artificial Sequence
  Recombinant protein
LEYSSYIRT

SEQ ID NO: 147 - protein, 122 aa, Artificial Sequence; identical to SEQ ID NO: 43
  Recombinant protein

SEQ ID NO: 148 - protein, 5 aa, Artificial Sequence
  Recombinant protein
DYAMH

SEQ ID NO: 149 - protein, 17 aa, Artificial Sequence
  Recombinant protein
GISWKGDIGGYVDSVKG

SEQ ID NO: 150 - protein, 13 aa, Artificial Sequence
  Recombinant protein
SYGSGSFYNAFDS

SEQ ID NO: 151 - protein, 107 aa, Artificial Sequence
  Recombinant protein
DIQMTQSPSTLSASVGDRVTITCRASQSISSWLAWYQQKPGKAPKFLIYKASRLDRGIPSRFSGSGSGTEFSLTISSLQPDDFATYYCLEYSSYIRTFGQGTKVEIK

SEQ ID NO: 152 - protein, 11 aa, Artificial Sequence
  Recombinant protein
RASQSISSWLA

SEQ ID NO: 153 - protein, 7 aa, Artificial Sequence
  Recombinant protein
KASRLDR

SEQ ID NO: 154 - protein, 9 aa, Artificial Sequence
  Recombinant protein
LEYSSYIRT

SEQ ID NO: 155 - protein, 122 aa, Artificial Sequence; identical to SEQ ID NO: 43
  Recombinant protein

SEQ ID NO: 156 - protein, 5 aa, Artificial Sequence
  Recombinant protein
DYAMH

SEQ ID NO: 157 - protein, 17 aa, Artificial Sequence
  Recombinant protein
GISWKGDIGGYVDSVKG

SEQ ID NO: 158 - protein, 13 aa, Artificial Sequence
  Recombinant protein
SYGSGSFYNAFDS

SEQ ID NO: 159 - protein, 107 aa, Artificial Sequence
  Recombinant protein
DIQMTQSPSTLSASVGDRVTITCRASQSISSWLAWYQQKPGKAPKFLIYKASRLDRGLPSRFSGSGSGTEFSLTISSLQPDDFATYYCLEYSSYIRTFGQGTKVEIK

SEQ ID NO: 160 - protein, 11 aa, Artificial Sequence
  Recombinant protein
RASQSISSWLA

SEQ ID NO: 161 - protein, 7 aa, Artificial Sequence
  Recombinant protein
KASRLDR

SEQ ID NO: 162 - protein, 9 aa, Artificial Sequence
  Recombinant protein
LEYSSYIRT

SEQ ID NO: 163 - protein, 122 aa, Artificial Sequence; identical to SEQ ID NO: 43
  Recombinant protein

SEQ ID NO: 164 - protein, 5 aa, Artificial Sequence
  Recombinant protein
DYAMH

SEQ ID NO: 165 - protein, 17 aa, Artificial Sequence
  Recombinant protein
GISWKGDIGGYVDSVKG

SEQ ID NO: 166 - protein, 13 aa, Artificial Sequence
  Recombinant protein
SYGSGSFYNAFDS

SEQ ID NO: 167 - protein, 107 aa, Artificial Sequence
  Recombinant protein
DIQMTQSPSTLSASVGDRVTITCRASQSISSWLAWYQQKPGKAPKFLIYKASRLDRGMPSRFSGSGSGTEFSLTISSLQPDDFATYYCLEYSSYIRTFGQGTKVEIK

SEQ ID NO: 168 - protein, 11 aa, Artificial Sequence
  Recombinant protein
RASQSISSWLA

SEQ ID NO: 169 - protein, 7 aa, Artificial Sequence
  Recombinant protein
KASRLDR

SEQ ID NO: 170 - protein, 9 aa, Artificial Sequence
  Recombinant protein
LEYSSYIRT

SEQ ID NO: 171 - protein, 122 aa, Artificial Sequence; identical to SEQ ID NO: 43
  Recombinant protein

SEQ ID NO: 172 - protein, 5 aa, Artificial Sequence
  Recombinant protein
DYAMH

SEQ ID NO: 173 - protein, 17 aa, Artificial Sequence
  Recombinant protein
GISWKGDIGGYVDSVKG

SEQ ID NO: 174 - protein, 13 aa, Artificial Sequence
  Recombinant protein
SYGSGSFYNAFDS

SEQ ID NO: 175 - protein, 107 aa, Artificial Sequence
  Recombinant protein
DIQMTQSPSTLSASVGDRVTITCRASQSISSWLAWYQQKPGKAPKFLIYKASRLDRGNPSRFSGSGSGTEFSLTISSLQPDDFATYYCLEYSSYIRTFGQGTKVEIK

SEQ ID NO: 176 - protein, 11 aa, Artificial Sequence
  Recombinant protein
RASQSISSWLA

SEQ ID NO: 177 - protein, 7 aa, Artificial Sequence
  Recombinant protein
KASRLDR

SEQ ID NO: 178 - protein, 9 aa, Artificial Sequence
  Recombinant protein
LEYSSYIRT

SEQ ID NO: 179 - protein, 122 aa, Artificial Sequence; identical to SEQ ID NO: 43
  Recombinant protein

SEQ ID NO: 180 - protein, 5 aa, Artificial Sequence
  Recombinant protein
DYAMH

SEQ ID NO: 181 - protein, 17 aa, Artificial Sequence
  Recombinant protein
GISWKGDIGGYVDSVKG

SEQ ID NO: 182 - protein, 13 aa, Artificial Sequence
  Recombinant protein
SYGSGSFYNAFDS

SEQ ID NO: 183 - protein, 107 aa, Artificial Sequence
  Recombinant protein
DIQMTQSPSTLSASVGDRVTITCRASQSISSWLAWYQQKPGKAPKFLIYKASRLDRGQPSRFSGSGSGTEFSLTISSLQPDDFATYYCLEYSSYIRTFGQGTKVEIK

SEQ ID NO: 184 - protein, 11 aa, Artificial Sequence
  Recombinant protein
RASQSISSWLA

SEQ ID NO: 185 - protein, 7 aa, Artificial Sequence
  Recombinant protein
KASRLDR

SEQ ID NO: 186 - protein, 9 aa, Artificial Sequence
  Recombinant protein
LEYSSYIRT

SEQ ID NO: 187 - protein, 122 aa, Artificial Sequence; identical to SEQ ID NO: 43
  Recombinant protein

SEQ ID NO: 188 - protein, 5 aa, Artificial Sequence
  Recombinant protein
DYAMH

SEQ ID NO: 189 - protein, 17 aa, Artificial Sequence
  Recombinant protein
GISWKGDIGGYVDSVKG

SEQ ID NO: 190 - protein, 13 aa, Artificial Sequence
  Recombinant protein
SYGSGSFYNAFDS

SEQ ID NO: 191 - protein, 107 aa, Artificial Sequence
  Recombinant protein
DIQMTQSPSTLSASVGDRVTITCRASQSISSWLAWYQQKPGKAPKFLIYKASRLDRGSPSRFSGSGSGTEFSLTISSLQPDDFATYYCLEYSSYIRTFGQGTKVEIK

SEQ ID NO: 192 - protein, 11 aa, Artificial Sequence
  Recombinant protein
RASQSISSWLA

SEQ ID NO: 193 - protein, 7 aa, Artificial Sequence
  Recombinant protein
KASRLDR

SEQ ID NO: 194 - protein, 9 aa, Artificial Sequence
  Recombinant protein
LEYSSYIRT

SEQ ID NO: 195 - protein, 122 aa, Artificial Sequence; identical to SEQ ID NO: 43
  Recombinant protein

SEQ ID NO: 196 - protein, 5 aa, Artificial Sequence
  Recombinant protein
DYAMH

SEQ ID NO: 197 - protein, 17 aa, Artificial Sequence
  Recombinant protein
GISWKGDIGGYVDSVKG

SEQ ID NO: 198 - protein, 13 aa, Artificial Sequence
  Recombinant protein
SYGSGSFYNAFDS

SEQ ID NO: 199 - protein, 107 aa, Artificial Sequence
  Recombinant protein
DIQMTQSPSTLSASVGDRVTITCRASQSISSWLAWYQQKPGKAPKFLIYKASRLDRGVPSRFSGSGSGTEFSLTISSLQPDDFATYYCLEYSSYIRTFGQGTKVEIK

SEQ ID NO: 200 - protein, 11 aa, Artificial Sequence
  Recombinant protein
RASQSISSWLA

SEQ ID NO: 201 - protein, 7 aa, Artificial Sequence
  Recombinant protein
KASRLDR

SEQ ID NO: 202 - protein, 9 aa, Artificial Sequence
  Recombinant protein
LEYSSYIRT

SEQ ID NO: 203 - protein, 122 aa, Artificial Sequence; identical to SEQ ID NO: 43
  Recombinant protein

SEQ ID NO: 204 - protein, 5 aa, Artificial Sequence
  Recombinant protein
DYAMH

SEQ ID NO: 205 - protein, 17 aa, Artificial Sequence
  Recombinant protein
GISWKGDIGGYVDSVKG

SEQ ID NO: 206 - protein, 13 aa, Artificial Sequence
  Recombinant protein
SYGSGSFYNAFDS

SEQ ID NO: 207 - protein, 107 aa, Artificial Sequence
  Recombinant protein
DIQMTQSPSTLSASVGDRVTITCRASQSISSWLAWYQQKPGKAPKFLIYKASRLDRGWPSRFSGSGSGTEFSLTISSLQPDDFATYYCLEYSSYIRTFGQGTKVEIK

SEQ ID NO: 208 - protein, 11 aa, Artificial Sequence
  Recombinant protein
RASQSISSWLA

SEQ ID NO: 209 - protein, 7 aa, Artificial Sequence
  Recombinant protein
KASRLDR

SEQ ID NO: 210 - protein, 9 aa, Artificial Sequence
  Recombinant protein
LEYSSYIRT

SEQ ID NO: 211 - protein, 122 aa, Artificial Sequence
  Recombinant protein
EVQLVESGGGLVQPGRSLRLSCAASKFTFHDYAMHWVRQAPGKGLEWVSGISWKGDIGGYADSVKGRFTISRDNAKNSLYLQMNSLRAEDTALYYCAKSYGSGSFYNAFDSWGQGTLVTVSS

SEQ ID NO: 212 - protein, 5 aa, Artificial Sequence
  Recombinant protein
DYAMH

SEQ ID NO: 213 - protein, 17 aa, Artificial Sequence
  Recombinant protein
GISWKGDIGGYADSVKG

SEQ ID NO: 214 - protein, 13 aa, Artificial Sequence
  Recombinant protein
SYGSGSFYNAFDS

SEQ ID NO: 215 - protein, 107 aa, Artificial Sequence; identical to SEQ ID NO: 127
  Recombinant protein

SEQ ID NO: 216 - protein, 11 aa, Artificial Sequence
  Recombinant protein
RASQSISSWLA

SEQ ID NO: 217 - protein, 7 aa, Artificial Sequence
  Recombinant protein
KASKLDR

SEQ ID NO: 218 - protein, 9 aa, Artificial Sequence
  Recombinant protein
LEYSSYIRT

SEQ ID NO: 219 - protein, 122 aa, Artificial Sequence; identical to SEQ ID NO: 211
  Recombinant protein

SEQ ID NO: 220 - protein, 5 aa, Artificial Sequence
  Recombinant protein
DYAMH

SEQ ID NO: 221 - protein, 17 aa, Artificial Sequence
  Recombinant protein
GISWKGDIGGYADSVKG

SEQ ID NO: 222 - protein, 13 aa, Artificial Sequence
  Recombinant protein
SYGSGSFYNAFDS

SEQ ID NO: 223 - protein, 107 aa, Artificial Sequence
  Recombinant protein
DIQMTQSPSTLSASVGDRVTITCRASQSISSWLAWYQQKPGKAPKFLIYKASKLERGTPSRFSGSGSGTEFTLTISSLQPDDFATYYCLEYSSYIRTFGQGTKVEIK

SEQ ID NO: 224 - protein, 11 aa, Artificial Sequence
  Recombinant protein
RASQSISSWLA

SEQ ID NO: 225 - protein, 7 aa, Artificial Sequence
  Recombinant protein
KASKLER

SEQ ID NO: 226 - protein, 9 aa, Artificial Sequence
  Recombinant protein
LEYSSYIRT

SEQ ID NO: 227 - protein, 122 aa, Artificial Sequence
  Recombinant protein
EVQLVESGGGLVQPGRSLRLSCAASKFTFHDYAMHWVRQAPGKGLEWVSGISWKGDIGGYVDSVKGRFTISRDNAKNSLYLQMNSLRAEDTALYYCAKSYGSGSFYNAFDSWGQGTLVTVSS

SEQ ID NO: 228 - protein, 5 aa, Artificial Sequence
  Recombinant protein
DYAMH

SEQ ID NO: 229 - protein, 17 aa, Artificial Sequence
  Recombinant protein
GISWKGDIGGYVDSVKG

SEQ ID NO: 230 - protein, 13 aa, Artificial Sequence
  Recombinant protein
SYGSGSFYNAFDS

SEQ ID NO: 231 - protein, 107 aa, Artificial Sequence; identical to SEQ ID NO: 127
  Recombinant protein

SEQ ID NO: 232 - protein, 11 aa, Artificial Sequence
  Recombinant protein
RASQSISSWLA

SEQ ID NO: 233 - protein, 7 aa, Artificial Sequence
  Recombinant protein
KASKLDR

SEQ ID NO: 234 - protein, 9 aa, Artificial Sequence
  Recombinant protein
LEYSSYIRT

SEQ ID NO: 235 - protein, 122 aa, Artificial Sequence; identical to SEQ ID NO: 227
  Recombinant protein

SEQ ID NO: 236 - protein, 5 aa, Artificial Sequence
  Recombinant protein
DYAMH

SEQ ID NO: 237 - protein, 17 aa, Artificial Sequence
  Recombinant protein
GISWKGDIGGYVDSVKG

SEQ ID NO: 238 - protein, 13 aa, Artificial Sequence
  Recombinant protein
SYGSGSFYNAFDS

SEQ ID NO: 239 - protein, 107 aa, Artificial Sequence; identical to SEQ ID NO: 223
  Recombinant protein

SEQ ID NO: 240 - protein, 11 aa, Artificial Sequence
  Recombinant protein
RASQSISSWLA

SEQ ID NO: 241 - protein, 7 aa, Artificial Sequence
  Recombinant protein
KASKLER

SEQ ID NO: 242 - protein, 9 aa, Artificial Sequence
  Recombinant protein
LEYSSYIRT

SEQ ID NO: 243 - protein, 122 aa, Artificial Sequence
  Recombinant protein
EVQLVESGGGLVQPGRSLRLSCAASKFTFHDYAMHWVRQVPGKGLEWVSGISWKGDIGGYVDSVKGRFTISRDNAKNSLYLQMNSLRAEDTALYYCAKSYGSGSFYNAFDSWGQGTLVTVSS

SEQ ID NO: 244 - protein, 5 aa, Artificial Sequence
  Recombinant protein
DYAMH

SEQ ID NO: 245 - protein, 17 aa, Artificial Sequence
  Recombinant protein
GISWKGDIGGYVDSVKG

SEQ ID NO: 246 - protein, 13 aa, Artificial Sequence
  Recombinant protein
SYGSGSFYNAFDS

SEQ ID NO: 247 - protein, 107 aa, Artificial Sequence; identical to SEQ ID NO: 127
  Recombinant protein

SEQ ID NO: 248 - protein, 11 aa, Artificial Sequence
  Recombinant protein
RASQSISSWLA

SEQ ID NO: 249 - protein, 7 aa, Artificial Sequence
  Recombinant protein
KASKLDR

SEQ ID NO: 250 - protein, 9 aa, Artificial Sequence
  Recombinant protein
LEYSSYIRT

SEQ ID NO: 251 - protein, 122 aa, Artificial Sequence; identical to SEQ ID NO: 243
  Recombinant protein

SEQ ID NO: 252 - protein, 5 aa, Artificial Sequence
  Recombinant protein
DYAMH

SEQ ID NO: 253 - protein, 17 aa, Artificial Sequence
  Recombinant protein
GISWKGDIGGYVDSVKG

SEQ ID NO: 254 - protein, 13 aa, Artificial Sequence
  Recombinant protein
SYGSGSFYNAFDS

SEQ ID NO: 255 - protein, 107 aa, Artificial Sequence; identical to SEQ ID NO: 223
  Recombinant protein

SEQ ID NO: 256 - protein, 11 aa, Artificial Sequence
  Recombinant protein
RASQSISSWLA

SEQ ID NO: 257 - protein, 7 aa, Artificial Sequence
  Recombinant protein
KASKLER

SEQ ID NO: 258 - protein, 9 aa, Artificial Sequence
  Recombinant protein
LEYSSYIRT

SEQ ID NO: 259 - protein, 122 aa, Artificial Sequence
  Recombinant protein
EVQLVESGGGLVQPGRSLKLSCAASKFTFHDYAMHWVRQAPGKGLEWVSGISWKGDIGGYVDSVKGRFTISRDNAKNSLYLQMNSLRAEDTALYYCAKSYGSGSFYNAFDSWGQGTLVTVSS

SEQ ID NO: 260 - protein, 5 aa, Artificial Sequence
  Recombinant protein
DYAMH

SEQ ID NO: 261 - protein, 17 aa, Artificial Sequence
  Recombinant protein
GISWKGDIGGYVDSVKG

SEQ ID NO: 262 - protein, 13 aa, Artificial Sequence
  Recombinant protein
SYGSGSFYNAFDS

SEQ ID NO: 263 - protein, 107 aa, Artificial Sequence; identical to SEQ ID NO: 127
  Recombinant protein

SEQ ID NO: 264 - protein, 11 aa, Artificial Sequence
  Recombinant protein
RASQSISSWLA

SEQ ID NO: 265 - protein, 7 aa, Artificial Sequence
  Recombinant protein
KASKLDR

SEQ ID NO: 266 - protein, 9 aa, Artificial Sequence
  Recombinant protein
LEYSSYIRT

SEQ ID NO: 267 - protein, 122 aa, Artificial Sequence; identical to SEQ ID NO: 259
  Recombinant protein

SEQ ID NO: 268 - protein, 5 aa, Artificial Sequence
  Recombinant protein
DYAMH

SEQ ID NO: 269 - protein, 17 aa, Artificial Sequence
  Recombinant protein
GISWKGDIGGYVDSVKG

SEQ ID NO: 270 - protein, 13 aa, Artificial Sequence
  Recombinant protein
SYGSGSFYNAFDS

SEQ ID NO: 271 - protein, 107 aa, Artificial Sequence; identical to SEQ ID NO: 223
  Recombinant protein

SEQ ID NO: 272 - protein, 11 aa, Artificial Sequence
  Recombinant protein
RASQSISSWLA

SEQ ID NO: 273 - protein, 7 aa, Artificial Sequence
  Recombinant protein
KASKLER

SEQ ID NO: 274 - protein, 9 aa, Artificial Sequence
  Recombinant protein
LEYSSYIRT

SEQ ID NO: 275 - protein, 122 aa, Artificial Sequence
  Recombinant protein
EVQLVESGGGLVQPGRSLRLSCAASGFTFHDYAMHWVRQVPGKGLEWVSGISWKGDIGGYVDSVKGRFTISRDNAKNSLYLQMNSLRAEDTALYYCAKSYGSGSFYNAFDSWGQGTLVTVSS

SEQ ID NO: 276 - protein, 5 aa, Artificial Sequence
  Recombinant protein
DYAMH

SEQ ID NO: 277 - protein, 17 aa, Artificial Sequence
  Recombinant protein
GISWKGDIGGYVDSVKG

SEQ ID NO: 278 - protein, 13 aa, Artificial Sequence
  Recombinant protein
SYGSGSFYNAFDS

SEQ ID NO: 279 - protein, 107 aa, Artificial Sequence; identical to SEQ ID NO: 127
  Recombinant protein

SEQ ID NO: 280 - protein, 11 aa, Artificial Sequence
  Recombinant protein
RASQSISSWLA

SEQ ID NO: 281 - protein, 7 aa, Artificial Sequence
  Recombinant protein
KASKLDR

SEQ ID NO: 282 - protein, 9 aa, Artificial Sequence
  Recombinant protein
LEYSSYIRT

SEQ ID NO: 283 - protein, 122 aa, Artificial Sequence; identical to SEQ ID NO: 275
  Recombinant protein

SEQ ID NO: 284 - protein, 5 aa, Artificial Sequence
  Recombinant protein
DYAMH

SEQ ID NO: 285 - protein, 17 aa, Artificial Sequence
  Recombinant protein
GISWKGDIGGYVDSVKG

SEQ ID NO: 286 - protein, 13 aa, Artificial Sequence
  Recombinant protein
SYGSGSFYNAFDS

SEQ ID NO: 287 - protein, 107 aa, Artificial Sequence; identical to SEQ ID NO: 223
  Recombinant protein

SEQ ID NO: 288 - protein, 11 aa, Artificial Sequence
  Recombinant protein
RASQSISSWLA

SEQ ID NO: 289 - protein, 7 aa, Artificial Sequence
  Recombinant protein
KASKLER

SEQ ID NO: 290 - protein, 9 aa, Artificial Sequence
  Recombinant protein
LEYSSYIRT

SEQ ID NO: 291 - protein, 122 aa, Artificial Sequence
  Recombinant protein
EVQLVESGGGLVQPGRSLKLSCAASGFTFHDYAMHWVRQVPGKGLEWVSGISWKGDIGGYVDSVKGRFTISRDNAKNSLYLQMNSLRAEDTALYYCAKSYGSGSFYNAFDSWGQGTLVTVSS

SEQ ID NO: 292 - protein, 5 aa, Artificial Sequence
  Recombinant protein
DYAMH

SEQ ID NO: 293 - protein, 17 aa, Artificial Sequence
  Recombinant protein
GISWKGDIGGYVDSVKG

SEQ ID NO: 294 - protein, 13 aa, Artificial Sequence
  Recombinant protein
SYGSGSFYNAFDS

SEQ ID NO: 295 - protein, 107 aa, Artificial Sequence; identical to SEQ ID NO: 127
  Recombinant protein

SEQ ID NO: 296 - protein, 11 aa, Artificial Sequence
  Recombinant protein
RASQSISSWLA

SEQ ID NO: 297 - protein, 7 aa, Artificial Sequence
  Recombinant protein
KASKLDR

SEQ ID NO: 298 - protein, 9 aa, Artificial Sequence
  Recombinant protein
LEYSSYIRT

SEQ ID NO: 299 - protein, 122 aa, Artificial Sequence; identical to SEQ ID NO: 291
  Recombinant protein

SEQ ID NO: 300 - protein, 5 aa, Artificial Sequence
  Recombinant protein
DYAMH

SEQ ID NO: 301 - protein, 17 aa, Artificial Sequence
  Recombinant protein
GISWKGDIGGYVDSVKG

SEQ ID NO: 302 - protein, 13 aa, Artificial Sequence
  Recombinant protein
SYGSGSFYNAFDS

SEQ ID NO: 303 - protein, 107 aa, Artificial Sequence; identical to SEQ ID NO: 223
  Recombinant protein

SEQ ID NO: 304 - protein, 11 aa, Artificial Sequence
  Recombinant protein
RASQSISSWLA

SEQ ID NO: 305 - protein, 7 aa, Artificial Sequence
  Recombinant protein
KASKLER

SEQ ID NO: 306 - protein, 9 aa, Artificial Sequence
  Recombinant protein
LEYSSYIRT

SEQ ID NO: 307 - protein, 122 aa, Artificial Sequence
  Recombinant protein
EVQLVESGGGLVQPGRSLRLSCAASGFKFHDYAMHWVRQVPGKGLEWVSGISWKGDIGGYVDSVKGRFTISRDNEKNSLYLQMNSLRAEDTALYYCVKSYGSGSFYNAFDSWGQGTLVTVSS

SEQ ID NO: 308 - protein, 5 aa, Artificial Sequence
  Recombinant protein
DYAMH

SEQ ID NO: 309 - protein, 17 aa, Artificial Sequence
  Recombinant protein
GISWKGDIGGYVDSVKG

SEQ ID NO: 310 - protein, 13 aa, Artificial Sequence
  Recombinant protein
SYGSGSFYNAFDS

SEQ ID NO: 311 - protein, 107 aa, Artificial Sequence; identical to SEQ ID NO: 71
  Recombinant protein

SEQ ID NO: 312 - protein, 11 aa, Artificial Sequence
  Recombinant protein
RASQSISSWLA

SEQ ID NO: 313 - protein, 7 aa, Artificial Sequence
  Recombinant protein
KASRLDR

SEQ ID NO: 314 - protein, 9 aa, Artificial Sequence
  Recombinant protein
LEYSSYIRT

SEQ ID NO: 315 - protein, 122 aa, Artificial Sequence
  Recombinant protein
EVQLVESGGGLVQPGRSLRLSCAASGFTFHDYAMHWVRQVPGKGLEWVSGISWKGDIGGYVDSVKGRFTISRDNDKNSLYLQMNSLRAEDTALYYCVKSYGSGSFYNAFDSWGQGTLVTVSS

SEQ ID NO: 316 - protein, 5 aa, Artificial Sequence
  Recombinant protein
DYAMH

SEQ ID NO: 317 - protein, 17 aa, Artificial Sequence
  Recombinant protein
GISWKGDIGGYVDSVKG

SEQ ID NO: 318 - protein, 13 aa, Artificial Sequence
  Recombinant protein
SYGSGSFYNAFDS

SEQ ID NO: 319 - protein, 107 aa, Artificial Sequence; identical to SEQ ID NO: 71
  Recombinant protein

SEQ ID NO: 320 - protein, 11 aa, Artificial Sequence
  Recombinant protein
RASQSISSWLA

SEQ ID NO: 321 - protein, 7 aa, Artificial Sequence
  Recombinant protein
KASRLDR

SEQ ID NO: 322 - protein, 9 aa, Artificial Sequence
  Recombinant protein
LEYSSYIRT

SEQ ID NO: 323 - protein, 122 aa, Artificial Sequence; identical to SEQ ID NO: 43
  Recombinant protein

SEQ ID NO: 324 - protein, 5 aa, Artificial Sequence
  Recombinant protein
DYAMH

SEQ ID NO: 325 - protein, 17 aa, Artificial Sequence
  Recombinant protein
GISWKGDIGGYVDSVKG

SEQ ID NO: 326 - protein, 13 aa, Artificial Sequence
  Recombinant protein
SYGSGSFYNAFDS

SEQ ID NO: 327 - protein, 107 aa, Artificial Sequence
  Recombinant protein
DIQMTQSPSTLSASVGDRVTITCRASQKISSWLAWYQQKPGKAPKFLIYKASRLDRGTPSRFSGSGSGTEFSLTISSLQPDDFATYYCLEYSSYIRTFGQGTKVEIK

SEQ ID NO: 328 - protein, 11 aa, Artificial Sequence
  Recombinant protein
RASQKISSWLA

SEQ ID NO: 329 - protein, 7 aa, Artificial Sequence
  Recombinant protein
KASRLDR

SEQ ID NO: 330 - protein, 9 aa, Artificial Sequence
  Recombinant protein
LEYSSYIRT

SEQ ID NO: 331 - protein, 122 aa, Artificial Sequence; identical to SEQ ID NO: 43
  Recombinant protein

SEQ ID NO: 332 - protein, 5 aa, Artificial Sequence
  Recombinant protein
DYAMH

SEQ ID NO: 333 - protein, 17 aa, Artificial Sequence
  Recombinant protein
GISWKGDIGGYVDSVKG

SEQ ID NO: 334 - protein, 13 aa, Artificial Sequence
  Recombinant protein
SYGSGSFYNAFDS

SEQ ID NO: 335 - protein, 107 aa, Artificial Sequence
  Recombinant protein
DIQMTQSPSTLSASVGDRVTITCRASQQISSWLAWYQQKPGKAPKFLIYKASRLDRGTPSRFSGSGSGTEFSLTISSLQPDDFATYYCLEYSSYIRTFGQGTKVEIK

SEQ ID NO: 336 - protein, 11 aa, Artificial Sequence
  Recombinant protein
RASQQISSWLA

SEQ ID NO: 337 - protein, 7 aa, Artificial Sequence
  Recombinant protein
KASRLDR

SEQ ID NO: 338 - protein, 9 aa, Artificial Sequence
  Recombinant protein
LEYSSYIRT

SEQ ID NO: 339 - protein, 122 aa, Artificial Sequence; identical to SEQ ID NO: 43
  Recombinant protein

SEQ ID NO: 340 - protein, 5 aa, Artificial Sequence
  Recombinant protein
DYAMH

SEQ ID NO: 341 - protein, 17 aa, Artificial Sequence
  Recombinant protein
GISWKGDIGGYVDSVKG

SEQ ID NO: 342 - protein, 13 aa, Artificial Sequence
  Recombinant protein
SYGSGSFYNAFDS

SEQ ID NO: 343 - protein, 107 aa, Artificial Sequence
  Recombinant protein
DIQMTQSPSTLSASVGDRVTITCRASQSIQSWLAWYQQKPGKAPKFLIYKASRLDRGTPSRFSGSGSGTEFSLTISSLQPDDFATYYCLEYSSYIRTFGQGTKVEIK

SEQ ID NO: 344 - protein, 11 aa, Artificial Sequence
  Recombinant protein
RASQSIQSWLA

SEQ ID NO: 345 - protein, 7 aa, Artificial Sequence
  Recombinant protein
KASRLDR

SEQ ID NO: 346 - protein, 9 aa, Artificial Sequence
  Recombinant protein
LEYSSYIRT

SEQ ID NO: 347 - protein, 122 aa, Artificial Sequence; identical to SEQ ID NO: 43
  Recombinant protein

SEQ ID NO: 348 - protein, 5 aa, Artificial Sequence
  Recombinant protein
DYAMH

SEQ ID NO: 349 - protein, 17 aa, Artificial Sequence
  Recombinant protein
GISWKGDIGGYVDSVKG

SEQ ID NO: 350 - protein, 13 aa, Artificial Sequence
  Recombinant protein
SYGSGSFYNAFDS

SEQ ID NO: 351 - protein, 107 aa, Artificial Sequence
  Recombinant protein
DIQMTQSPSTLSASVGDRVTITCRASQSISSWLAWYQQKPGKAPKFLIYKASRLDKGTPSRFSGSGSGTEFSLTISSLQPDDFATYYCLEYSSYIRTFGQGTKVEIK

SEQ ID NO: 352 - protein, 11 aa, Artificial Sequence
  Recombinant protein
RASQSISSWLA

SEQ ID NO: 353 - protein, 7 aa, Artificial Sequence
  Recombinant protein
KASRLDK

SEQ ID NO: 354 - protein, 9 aa, Artificial Sequence
  Recombinant protein
LEYSSYIRT

SEQ ID NO: 355 - protein, 122 aa, Artificial Sequence; identical to SEQ ID NO: 43
  Recombinant protein

SEQ ID NO: 356 - protein, 5 aa, Artificial Sequence
  Recombinant protein
DYAMH

SEQ ID NO: 357 - protein, 17 aa, Artificial Sequence
  Recombinant protein
GISWKGDIGGYVDSVKG

SEQ ID NO: 358 - protein, 13 aa, Artificial Sequence
  Recombinant protein
SYGSGSFYNAFDS

SEQ ID NO: 359 - protein, 107 aa, Artificial Sequence
  Recombinant protein
DIQMTQSPSTLSASVGDRVTITCRASQSISSWLAWYQQKPGKAPKFLIYKASRLDRGTPKRFSGSGSGTEFSLTISSLQPDDFATYYCLEYSSYIRTFGQGTKVEIK

SEQ ID NO: 360 - protein, 11 aa, Artificial Sequence
  Recombinant protein
RASQSISSWLA

SEQ ID NO: 361 - protein, 7 aa, Artificial Sequence
  Recombinant protein
KASRLDR

SEQ ID NO: 362 - protein, 9 aa, Artificial Sequence
  Recombinant protein
LEYSSYIRT

SEQ ID NO: 363 - protein, 122 aa, Artificial Sequence; identical to SEQ ID NO: 43
  Recombinant protein

SEQ ID NO: 364 - protein, 5 aa, Artificial Sequence
  Recombinant protein
DYAMH

SEQ ID NO: 365 - protein, 17 aa, Artificial Sequence
  Recombinant protein
GISWKGDIGGYVDSVKG

SEQ ID NO: 366 - protein, 13 aa, Artificial Sequence
  Recombinant protein
SYGSGSFYNAFDS

SEQ ID NO: 367 - protein, 107 aa, Artificial Sequence
  Recombinant protein
DIQMTQSPSTLSASVGDRVTITCRASQSISSWLAWYQQKPGKAPKFLIYKASRLDRGTPSRFSGSGSGTEFSLTISELQPDDFATYYCLEYSSYIRTFGQGTKVEIK

SEQ ID NO: 368 - protein, 11 aa, Artificial Sequence
  Recombinant protein
RASQSISSWLA

SEQ ID NO: 369 - protein, 7 aa, Artificial Sequence
  Recombinant protein
KASRLDR

SEQ ID NO: 370 - protein, 9 aa, Artificial Sequence
  Recombinant protein
LEYSSYIRT

SEQ ID NO: 371 - protein, 122 aa, Artificial Sequence; identical to SEQ ID NO: 43
  Recombinant protein

SEQ ID NO: 372 - protein, 5 aa, Artificial Sequence
  Recombinant protein
DYAMH

SEQ ID NO: 373 - protein, 17 aa, Artificial Sequence
  Recombinant protein
GISWKGDIGGYVDSVKG

SEQ ID NO: 374 - protein, 13 aa, Artificial Sequence
  Recombinant protein
SYGSGSFYNAFDS

SEQ ID NO: 375 - protein, 107 aa, Artificial Sequence
  Recombinant protein
DIQMTQSPSTLSASVGDRVTITCRASQSISSWLAWYQQKPGKAPKFLIYKASRLDRGTPSRFSGSGSGTEFSLTISSLTPDDFATYYCLEYSSYIRTFGQGTKVEIK

SEQ ID NO: 376 - protein, 11 aa, Artificial Sequence
  Recombinant protein
RASQSISSWLA

SEQ ID NO: 377 - protein, 7 aa, Artificial Sequence
  Recombinant protein
KASRLDR

SEQ ID NO: 378 - protein, 9 aa, Artificial Sequence
  Recombinant protein
LEYSSYIRT

SEQ ID NO: 379 - protein, 122 aa, Artificial Sequence; identical to SEQ ID NO: 43
  Recombinant protein

SEQ ID NO: 380 - protein, 5 aa, Artificial Sequence
  Recombinant protein
DYAMH

SEQ ID NO: 381 - protein, 17 aa, Artificial Sequence
  Recombinant protein
GISWKGDIGGYVDSVKG

SEQ ID NO: 382 - protein, 13 aa, Artificial Sequence
  Recombinant protein
SYGSGSFYNAFDS

SEQ ID NO: 383 - protein, 107 aa, Artificial Sequence
  Recombinant protein
DIQMTQSPSTLSASVGDRVTITCRASQSISSWLAWYQQKPGKAPKFLIYKASRLDRGTPSRFSGSGSGTEFSLTISSLSPDDFATYYCLEYSSYIRTFGQGTKVEIK

SEQ ID NO: 384 - protein, 11 aa, Artificial Sequence
  Recombinant protein
RASQSISSWLA

SEQ ID NO: 385 - protein, 7 aa, Artificial Sequence
  Recombinant protein
KASRLDR

SEQ ID NO: 386 - protein, 9 aa, Artificial Sequence
  Recombinant protein
LEYSSYIRT

SEQ ID NO: 387 - protein, 122 aa, Artificial Sequence; identical to SEQ ID NO: 43
  Recombinant protein

SEQ ID NO: 388 - protein, 5 aa, Artificial Sequence
  Recombinant protein
DYAMH

SEQ ID NO: 389 - protein, 17 aa, Artificial Sequence
  Recombinant protein
GISWKGDIGGYVDSVKG

SEQ ID NO: 390 - protein, 13 aa, Artificial Sequence
  Recombinant protein
SYGSGSFYNAFDS

SEQ ID NO: 391 - protein, 107 aa, Artificial Sequence
  Recombinant protein
DIQMTQSPSTLSASVGDRVTITCRASQSISSWLAWYQQKPGKAPKFLIYKASRLDRGTPSRFSGSGSGTEFSLTISSLQPDDFATYYCLEYQSYIRTFGQGTKVEIK

SEQ ID NO: 392 - protein, 11 aa, Artificial Sequence
  Recombinant protein
RASQSISSWLA

SEQ ID NO: 393 - protein, 7 aa, Artificial Sequence
  Recombinant protein
KASRLDR

SEQ ID NO: 394 - protein, 9 aa, Artificial Sequence
  Recombinant protein
LEYQSYIRT

SEQ ID NO: 395 - protein, 122 aa, Artificial Sequence; identical to SEQ ID NO: 43
  Recombinant protein

SEQ ID NO: 396 - protein, 5 aa, Artificial Sequence
  Recombinant protein
DYAMH

SEQ ID NO: 397 - protein, 17 aa, Artificial Sequence
  Recombinant protein
GISWKGDIGGYVDSVKG

SEQ ID NO: 398 - protein, 13 aa, Artificial Sequence
  Recombinant protein
SYGSGSFYNAFDS

SEQ ID NO: 399 - protein, 107 aa, Artificial Sequence
  Recombinant protein
DIQMTQSPSTLSASVGDRVTITCRASQSISSWLAWYQQKPGKAPKFLIYKASRLDRGTPSRFSGSGSGTEFSLTISSLQPDDFATYYCLEYKSYIRTFGQGTKVEIK

SEQ ID NO: 400 - protein, 11 aa, Artificial Sequence
  Recombinant protein
RASQSISSWLA

SEQ ID NO: 401 - protein, 7 aa, Artificial Sequence
  Recombinant protein
KASRLDR

SEQ ID NO: 402 - protein, 9 aa, Artificial Sequence
  Recombinant protein
LEYKSYIRT

SEQ ID NO: 403 - protein, 122 aa, Artificial Sequence; identical to SEQ ID NO: 43
  Recombinant protein

SEQ ID NO: 404 - protein, 5 aa, Artificial Sequence
  Recombinant protein
DYAMH

SEQ ID NO: 405 - protein, 17 aa, Artificial Sequence
  Recombinant protein
GISWKGDIGGYVDSVKG

SEQ ID NO: 406 - protein, 13 aa, Artificial Sequence
  Recombinant protein
SYGSGSFYNAFDS

SEQ ID NO: 407 - protein, 107 aa, Artificial Sequence
  Recombinant protein
DIQMTQSPSTLSASVGDRVTITCRASQSISSWLAWYQQKPGKAPKFLIYKASRLDRGTPSRFSGSGSGTEFSLTISSLQPDDFATYYCLEYSSWIRTFGQGTKVEIK

SEQ ID NO: 408 - protein, 11 aa, Artificial Sequence
  Recombinant protein
RASQSISSWLA

SEQ ID NO: 409 - protein, 7 aa, Artificial Sequence
  Recombinant protein
KASRLDR

SEQ ID NO: 410 - protein, 9 aa, Artificial Sequence
  Recombinant protein
LEYSSWIRT

SEQ ID NO: 411 - protein, 122 aa, Artificial Sequence; identical to SEQ ID NO: 43
  Recombinant protein

SEQ ID NO: 412 - protein, 5 aa, Artificial Sequence
  Recombinant protein
DYAMH

SEQ ID NO: 413 - protein, 17 aa, Artificial Sequence
  Recombinant protein
GISWKGDIGGYVDSVKG

SEQ ID NO: 414 - protein, 13 aa, Artificial Sequence
  Recombinant protein
SYGSGSFYNAFDS

SEQ ID NO: 415 - protein, 107 aa, Artificial Sequence
  Recombinant protein
DIQMTQSPSTLSASVGDRVTITCRASQSISSWLAWYQQKPGKAPKFLIYKASRLDRGTPSRFSGSGSGTEFSLTIYDLQPDDFATYYCLEYRSYIRTFGQGTKVEIK

SEQ ID NO: 416 - protein, 11 aa, Artificial Sequence
  Recombinant protein
RASQSISSWLA

SEQ ID NO: 417 - protein, 7 aa, Artificial Sequence
  Recombinant protein
KASRLDR

SEQ ID NO: 418 - protein, 9 aa, Artificial Sequence
  Recombinant protein
LEYRSYIRT

SEQ ID NO: 419 - protein, 122 aa, Artificial Sequence; identical to SEQ ID NO: 43
  Recombinant protein

SEQ ID NO: 420 - protein, 5 aa, Artificial Sequence
  Recombinant protein
DYAMH

SEQ ID NO: 421 - protein, 17 aa, Artificial Sequence
  Recombinant protein
GISWKGDIGGYVDSVKG

SEQ ID NO: 422 - protein, 13 aa, Artificial Sequence
  Recombinant protein
SYGSGSFYNAFDS

SEQ ID NO: 423 - protein, 107 aa, Artificial Sequence
  Recombinant protein
DIQMTQSPSTLSASVGDRVTITCRASQSISSWLAWYQQKPGKAPKFLIYKASRLDRGTPQRFSGSGSGTEFSLTIYSLQPDDFATYYCLEYNSYIRTFGQGTKVEIK

SEQ ID NO: 424 - protein, 11 aa, Artificial Sequence
  Recombinant protein
RASQSISSWLA

SEQ ID NO: 425 - protein, 7 aa, Artificial Sequence
  Recombinant protein
KASRLDR

SEQ ID NO: 426 - protein, 9 aa, Artificial Sequence
  Recombinant protein
LEYNSYIRT

SEQ ID NO: 427 - protein, 122 aa, Artificial Sequence; identical to SEQ ID NO: 35
  Recombinant protein

SEQ ID NO: 428 - protein, 5 aa, Artificial Sequence
  Recombinant protein
DYAMH

SEQ ID NO: 429 - protein, 17 aa, Artificial Sequence
  Recombinant protein
GISWKGDIGGYVDSVKG

SEQ ID NO: 430 - protein, 13 aa, Artificial Sequence
  Recombinant protein
SYGSGSFYNAFDS

SEQ ID NO: 431 - protein, 107 aa, Artificial Sequence
  Recombinant protein
DIQMTQSPSTLSASAGDRVTITCRASQSISSWLAWYQQKPGKAPKFLIYKASKLDRGTPSRFSGSGSGTEFSLTISSLQPDDFATYYCLEYSSYIRTFGQGTKVEIK

SEQ ID NO: 432 - protein, 11 aa, Artificial Sequence
  Recombinant protein
RASQSISSWLA

SEQ ID NO: 433 - protein, 7 aa, Artificial Sequence
  Recombinant protein
KASKLDR

SEQ ID NO: 434 - protein, 9 aa, Artificial Sequence
  Recombinant protein
LEYSSYIRT

SEQ ID NO: 435 - protein, 122 aa, Artificial Sequence; identical to SEQ ID NO: 3
  Recombinant protein

SEQ ID NO: 436 - protein, 5 aa, Artificial Sequence
  Recombinant protein
DYAMH

SEQ ID NO: 437 - protein, 17 aa, Artificial Sequence
  Recombinant protein
GISWRGDIGGYVDSVKG

SEQ ID NO: 438 - protein, 13 aa, Artificial Sequence
  Recombinant protein
SYGSGSFYNAFDS

SEQ ID NO: 439 - protein, 107 aa, Artificial Sequence; identical to SEQ ID NO: 431
  Recombinant protein

SEQ ID NO: 440 - protein, 11 aa, Artificial Sequence
  Recombinant protein
RASQSISSWLA

SEQ ID NO: 441 - protein, 7 aa, Artificial Sequence
  Recombinant protein
KASKLDR

SEQ ID NO: 442 - protein, 9 aa, Artificial Sequence
  Recombinant protein
LEYSSYIRT

SEQ ID NO: 443 - protein, 122 aa, Artificial Sequence; identical to SEQ ID NO: 3
  Recombinant protein

SEQ ID NO: 444 - protein, 5 aa, Artificial Sequence
  Recombinant protein
DYAMH

SEQ ID NO: 445 - protein, 17 aa, Artificial Sequence
  Recombinant protein
GISWRGDIGGYVDSVKG

SEQ ID NO: 446 - protein, 13 aa, Artificial Sequence
  Recombinant protein
SYGSGSFYNAFDS

SEQ ID NO: 447 - protein, 107 aa, Artificial Sequence; identical to SEQ ID NO: 47
  Recombinant protein

SEQ ID NO: 448 - protein, 11 aa, Artificial Sequence
  Recombinant protein
RASQSISSWLA

SEQ ID NO: 449 - protein, 7 aa, Artificial Sequence
  Recombinant protein
KASRLER

SEQ ID NO: 450 - protein, 9 aa, Artificial Sequence
  Recombinant protein
LEYSSYIRT

SEQ ID NO: 451 - protein, 122 aa, Artificial Sequence; identical to SEQ ID NO: 123
  Recombinant protein

SEQ ID NO: 452 - protein, 5 aa, Artificial Sequence
  Recombinant protein
DYAMH

SEQ ID NO: 453 - protein, 17 aa, Artificial Sequence
  Recombinant protein
GISWKGDIGGYVDSVKG

SEQ ID NO: 454 - protein, 13 aa, Artificial Sequence
  Recombinant protein
SYGSGSFYNAFDS

SEQ ID NO: 455 - protein, 107 aa, Artificial Sequence; identical to SEQ ID NO: 23
  Recombinant protein

SEQ ID NO: 456 - protein, 11 aa, Artificial Sequence
  Recombinant protein
RASQSISSWLA

SEQ ID NO: 457 - protein, 7 aa, Artificial Sequence
  Recombinant protein
KASRLDR

SEQ ID NO: 458 - protein, 9 aa, Artificial Sequence
  Recombinant protein
LEYSSYIRT

SEQ ID NO: 459 - protein, 120 aa, Artificial Sequence
  Recombinant protein
EVQLVQSGAEVKKPGESLRISCKGSGYSFTTSWIVWVRQMPGKGLEWMGMIDPSDSFTSYSPSFQGHVTISADKSISTAYLQWSSLKASDTAMYYCARLHYYHSEEFDVWGQGTLVTVSS

SEQ ID NO: 460 - protein, 5 aa, Artificial Sequence
  Recombinant protein
TSWIV

SEQ ID NO: 461 - protein, 17 aa, Artificial Sequence
  Recombinant protein
MIDPSDSFTSYSPSFQG

SEQ ID NO: 462 - protein, 11 aa, Artificial Sequence
  Recombinant protein
LHYYHSEEFDV

SEQ ID NO: 463 - protein, 109 aa, Artificial Sequence
  Recombinant protein
EIVLTQSPGTLSLSPGERATLSCRASQSVSSSYLAWYQQKPGQAPRLLIYGASSRARGIPDRFSGSGSGTDFTLTISRLEPEDFAVYYCQQFGSSRLFTFGQGTKLEIK

SEQ ID NO: 464 - protein, 12 aa, Artificial Sequence
  Recombinant protein
RASQSVSSSYLA

SEQ ID NO: 465 - protein, 7 aa, Artificial Sequence
  Recombinant protein
GASSRAR

SEQ ID NO: 466 - protein, 10 aa, Artificial Sequence
  Recombinant protein
QQFGSSRLFT

SEQ ID NO: 467 - protein, 120 aa, Artificial Sequence
  Recombinant protein
EVQLVQSGAEVKKPGESLRISCKGSGYSFSTSWIVWVRQMPGKGLEWMGMIDPSDSFTSYSPSFQGHVTISADKSISTAYLQWSSLKASDTAMYYCARLHYYNSEEFDVWGQGTLVTVSS

SEQ ID NO: 468 - protein, 5 aa, Artificial Sequence
  Recombinant protein
TSWIV

SEQ ID NO: 469 - protein, 17 aa, Artificial Sequence
  Recombinant protein
MIDPSDSFTSYSPSFQG

SEQ ID NO: 470 - protein, 11 aa, Artificial Sequence
  Recombinant protein
LHYYNSEEFDV

SEQ ID NO: 471 - protein, 109 aa, Artificial Sequence
  Recombinant protein
EIVLTQSPGTLSLSPGERATLSCRASQSVSSSYLAWYQQKPGQAPRLLIYGQSSRTRGIPDRFSGSGSGTDFTLTISRLEPEDFAVYYCQQFGDSQLFTFGQGTKLEIK

SEQ ID NO: 472 - protein, 12 aa, Artificial Sequence
  Recombinant protein
RASQSVSSSYLA

SEQ ID NO: 473 - protein, 7 aa, Artificial Sequence
  Recombinant protein
GQSSRTR

SEQ ID NO: 474 - protein, 10 aa, Artificial Sequence
  Recombinant protein
QQFGDSQLFT

SEQ ID NO: 475 - protein, 120 aa, Artificial Sequence; identical to SEQ ID NO: 467
  Recombinant protein

SEQ ID NO: 476 - protein, 5 aa, Artificial Sequence
  Recombinant protein
TSWIV

SEQ ID NO: 477 - protein, 17 aa, Artificial Sequence
  Recombinant protein
MIDPSDSFTSYSPSFQG

SEQ ID NO: 478 - protein, 11 aa, Artificial Sequence
  Recombinant protein
LHYYNSEEFDV

SEQ ID NO: 479 - protein, 109 aa, Artificial Sequence
  Recombinant protein
EIVLTQSPGTLSLSAGERATLSCRASQSVSSSYLAWYQQKPGQAPRLLIYGASSRARGIPDRFSGSGSGTDFTLTISRLEPEDFAVYYCQQFGSSRLFTFGQGTKLEIK

SEQ ID NO: 480 - protein, 12 aa, Artificial Sequence
  Recombinant protein
RASQSVSSSYLA

SEQ ID NO: 481 - protein, 7 aa, Artificial Sequence
  Recombinant protein
GASSRAR

SEQ ID NO: 482 - protein, 10 aa, Artificial Sequence
  Recombinant protein
QQFGSSRLFT

SEQ ID NO: 483 - protein, 120 aa, Artificial Sequence
  Recombinant protein
EVQLVQSGAEVKKPGESLRISCKGSGYSFSTSWISWVRQMPGKGLEWMGMIDPSDSYTSYSPSFQGHVTISADKSISTAYLQWSSLKASDTAMYYCARLHYYNSEEFDVWGQGTMVTVSS

SEQ ID NO: 484 - protein, 5 aa, Artificial Sequence
  Recombinant protein
TSWIS

SEQ ID NO: 485 - protein, 17 aa, Artificial Sequence
  Recombinant protein
MIDPSDSYTSYSPSFQG

SEQ ID NO: 486 - protein, 11 aa, Artificial Sequence
  Recombinant protein
LHYYNSEEFDV

SEQ ID NO: 487 - protein, 109 aa, Artificial Sequence
  Recombinant protein
EIVLTQSPGTLSLSPGERATLSCRASQSVSSSYLAWYQQKPGQAPRLLIYGQSSRTRGIPDRFSGSGSGTDFTLTISRLEPEDFAVYYCQQYGDSQLFTFGQGTKLEIK

SEQ ID NO: 488 - protein, 12 aa, Artificial Sequence
  Recombinant protein
RASQSVSSSYLA

SEQ ID NO: 489 - protein, 7 aa, Artificial Sequence
  Recombinant protein
GQSSRTR

SEQ ID NO: 490 - protein, 10 aa, Artificial Sequence
  Recombinant protein
QQYGDSQLFT

SEQ ID NO: 491 - protein, 120 aa, Artificial Sequence
  Recombinant protein
EVQLVQSGAEVKKPGESLRISCKGSGYSFSTSWISWVRQMPGKGLEWMGMIDPSDSFTSYSPSFQGHVTISADKSISTAYLQWSSLKASDTAMYYCARLHYYNSEEFDVWGQGTLVTVSS

SEQ ID NO: 492 - protein, 5 aa, Artificial Sequence
  Recombinant protein
TSWIS

SEQ ID NO: 493 - protein, 17 aa, Artificial Sequence
  Recombinant protein
MIDPSDSFTSYSPSFQG

SEQ ID NO: 494 - protein, 11 aa, Artificial Sequence
  Recombinant protein
LHYYNSEEFDV

SEQ ID NO: 495 - protein, 109 aa, Artificial Sequence; identical to SEQ ID NO: 487
  Recombinant protein

SEQ ID NO: 496 - protein, 12 aa, Artificial Sequence
  Recombinant protein
RASQSVSSSYLA

SEQ ID NO: 497 - protein, 7 aa, Artificial Sequence
  Recombinant protein
GQSSRTR

SEQ ID NO: 498 - protein, 10 aa, Artificial Sequence
  Recombinant protein
QQYGDSQLFT

SEQ ID NO: 499 - protein, 120 aa, Artificial Sequence; identical to SEQ ID NO: 483
  Recombinant protein

SEQ ID NO: 500 - protein, 5 aa, Artificial Sequence
  Recombinant protein
TSWIS

SEQ ID NO: 501 - protein, 17 aa, Artificial Sequence
  Recombinant protein
MIDPSDSYTSYSPSFQG

SEQ ID NO: 502 - protein, 11 aa, Artificial Sequence
  Recombinant protein
LHYYNSEEFDV

SEQ ID NO: 503 - protein, 109 aa, Artificial Sequence; identical to SEQ ID NO: 471
  Recombinant protein

SEQ ID NO: 504 - protein, 12 aa, Artificial Sequence
  Recombinant protein
RASQSVSSSYLA

SEQ ID NO: 505 - protein, 7 aa, Artificial Sequence
  Recombinant protein
GQSSRTR

SEQ ID NO: 506 - protein, 10 aa, Artificial Sequence
  Recombinant protein
QQFGDSQLFT

SEQ ID NO: 507 - protein, 120 aa, Artificial Sequence
  Recombinant protein
EVQLVQSGAEVKKPGESLRISCKGSGYSFTTSWISWVRQMPGKGLEWMGMIDPSDSFTSYSPSFQGHVTISADKSISTAYLQWSSLKASDTAMYYCARLHYYHSEEFDVWGQGTMVTVSS

SEQ ID NO: 508 - protein, 5 aa, Artificial Sequence
  Recombinant protein
TSWIS

SEQ ID NO: 509 - protein, 17 aa, Artificial Sequence
  Recombinant protein
MIDPSDSFTSYSPSFQG

SEQ ID NO: 510 - protein, 11 aa, Artificial Sequence
  Recombinant protein
LHYYHSEEFDV

SEQ ID NO: 511 - protein, 109 aa, Artificial Sequence; identical to SEQ ID NO: 471
  Recombinant protein

SEQ ID NO: 512 - protein, 12 aa, Artificial Sequence
  Recombinant protein
RASQSVSSSYLA

SEQ ID NO: 513 - protein, 7 aa, Artificial Sequence
  Recombinant protein
GQSSRTR

SEQ ID NO: 514 - protein, 10 aa, Artificial Sequence
  Recombinant protein
QQFGDSQLFT

SEQ ID NO: 515 - protein, 120 aa, Artificial Sequence
  Recombinant protein
EVQLVQSGAEVKKPGESLRISCKGSGYSFTTSWISWVRQMPGKGLEWMGMIDPSDSYTSYSPSFQGHVTISADKSISTAYLQWSSLKASDTAMYYCARLHYYHSEEFDVWGQGTLVTVSS

SEQ ID NO: 516 - protein, 5 aa, Artificial Sequence
  Recombinant protein
TSWIS

SEQ ID NO: 517 - protein, 17 aa, Artificial Sequence
  Recombinant protein
MIDPSDSYTSYSPSFQG

SEQ ID NO: 518 - protein, 11 aa, Artificial Sequence
  Recombinant protein
LHYYHSEEFDV

SEQ ID NO: 519 - protein, 109 aa, Artificial Sequence; identical to SEQ ID NO: 471
  Recombinant protein

SEQ ID NO: 520 - protein, 12 aa, Artificial Sequence
  Recombinant protein
RASQSVSSSYLA

SEQ ID NO: 521 - protein, 7 aa, Artificial Sequence
  Recombinant protein
GQSSRTR

SEQ ID NO: 522 - protein, 10 aa, Artificial Sequence
  Recombinant protein
QQFGDSQLFT

SEQ ID NO: 523 - protein, 120 aa, Artificial Sequence
  Recombinant protein
EVQLVQSGAEVKKPGESLRISCKGSGYSFTTSWISWVRQMPGKGLEWMGMIDPSDSYTSYSPSFQGHVTISADKSISTAYLQWSSLKASDTAMYYCARLHYYHSEEFDVWGQGTMVTVSS

SEQ ID NO: 524 - protein, 5 aa, Artificial Sequence
  Recombinant protein
TSWIS

SEQ ID NO: 525 - protein, 17 aa, Artificial Sequence
  Recombinant protein
MIDPSDSYTSYSPSFQG

SEQ ID NO: 526 - protein, 11 aa, Artificial Sequence
  Recombinant protein
LHYYHSEEFDV

SEQ ID NO: 527 - protein, 109 aa, Artificial Sequence; identical to SEQ ID NO: 471
  Recombinant protein

SEQ ID NO: 528 - protein, 12 aa, Artificial Sequence
  Recombinant protein
RASQSVSSSYLA

SEQ ID NO: 529 - protein, 7 aa, Artificial Sequence
  Recombinant protein
GQSSRTR

SEQ ID NO: 530 - protein, 10 aa, Artificial Sequence
  Recombinant protein
QQFGDSQLFT

SEQ ID NO: 531 - protein, 120 aa, Artificial Sequence; identical to SEQ ID NO: 491
  Recombinant protein

SEQ ID NO: 532 - protein, 5 aa, Artificial Sequence
  Recombinant protein
TSWIS

SEQ ID NO: 533 - protein, 17 aa, Artificial Sequence
  Recombinant protein
MIDPSDSFTSYSPSFQG

SEQ ID NO: 534 - protein, 11 aa, Artificial Sequence
  Recombinant protein
LHYYNSEEFDV

SEQ ID NO: 535 - protein, 109 aa, Artificial Sequence; identical to SEQ ID NO: 471
  Recombinant protein

SEQ ID NO: 536 - protein, 12 aa, Artificial Sequence
  Recombinant protein
RASQSVSSSYLA

SEQ ID NO: 537 - protein, 7 aa, Artificial Sequence
  Recombinant protein
GQSSRTR

SEQ ID NO: 538 - protein, 10 aa, Artificial Sequence
  Recombinant protein
QQFGDSQLFT

SEQ ID NO: 539 - protein, 120 aa, Artificial Sequence
  Recombinant protein
EVQLVQSGAEVKKPGESLRISCKGSGYSFSTSWIVWVRQMPGKGLEWMGMIDPSDSYTSYSPSFQGHVTISADKSISTAYLQWSSLKASDTAMYYCARLHYYNSEEFDVWGQGTLVTVSS

SEQ ID NO: 540 - protein, 5 aa, Artificial Sequence
  Recombinant protein
TSWIV

SEQ ID NO: 541 - protein, 17 aa, Artificial Sequence
  Recombinant protein
MIDPSDSYTSYSPSFQG

SEQ ID NO: 542 - protein, 11 aa, Artificial Sequence
  Recombinant protein
LHYYNSEEFDV

SEQ ID NO: 543 - protein, 109 aa, Artificial Sequence; identical to SEQ ID NO: 471
  Recombinant protein

SEQ ID NO: 544 - protein, 12 aa, Artificial Sequence
  Recombinant protein
RASQSVSSSYLA

SEQ ID NO: 545 - protein, 7 aa, Artificial Sequence
  Recombinant protein
GQSSRTR

SEQ ID NO: 546 - protein, 10 aa, Artificial Sequence
  Recombinant protein
QQFGDSQLFT

SEQ ID NO: 547 - protein, 120 aa, Artificial Sequence
  Recombinant protein
EVQLVQSGAEVKKPGESLRISCKGSGYSFSTSWIVWVRQMPGKGLEWMGMIDPSDSYTSYSPSFQGHVTISADKSISTAYLQWSSLKASDTAMYYCARLHYYNSEEFDVWGQGTMVTVSS

SEQ ID NO: 548 - protein, 5 aa, Artificial Sequence
  Recombinant protein
TSWIV

SEQ ID NO: 549 - protein, 17 aa, Artificial Sequence
  Recombinant protein
MIDPSDSYTSYSPSFQG

SEQ ID NO: 550 - protein, 11 aa, Artificial Sequence
  Recombinant protein
LHYYNSEEFDV

SEQ ID NO: 551 - protein, 109 aa, Artificial Sequence; identical to SEQ ID NO: 471
  Recombinant protein

SEQ ID NO: 552 - protein, 12 aa, Artificial Sequence
  Recombinant protein
RASQSVSSSYLA

SEQ ID NO: 553 - protein, 7 aa, Artificial Sequence
  Recombinant protein
GQSSRTR

SEQ ID NO: 554 - protein, 10 aa, Artificial Sequence
  Recombinant protein
QQFGDSQLFT

SEQ ID NO: 555 - protein, 120 aa, Artificial Sequence; identical to SEQ ID NO: 467
  Recombinant protein

SEQ ID NO: 556 - protein, 5 aa, Artificial Sequence
  Recombinant protein
TSWIV

SEQ ID NO: 557 - protein, 17 aa, Artificial Sequence
  Recombinant protein
MIDPSDSFTSYSPSFQG

SEQ ID NO: 558 - protein, 11 aa, Artificial Sequence
  Recombinant protein
LHYYNSEEFDV

SEQ ID NO: 559 - protein, 109 aa, Artificial Sequence; identical to SEQ ID NO: 487
  Recombinant protein

SEQ ID NO: 560 - protein, 12 aa, Artificial Sequence
  Recombinant protein
RASQSVSSSYLA

SEQ ID NO: 561 - protein, 7 aa, Artificial Sequence
  Recombinant protein
GQSSRTR

SEQ ID NO: 562 - protein, 10 aa, Artificial Sequence
  Recombinant protein
QQYGDSQLFT

SEQ ID NO: 563 - protein, 120 aa, Artificial Sequence; identical to SEQ ID NO: 507
  Recombinant protein

SEQ ID NO: 564 - protein, 5 aa, Artificial Sequence
  Recombinant protein
TSWIS

SEQ ID NO: 565 - protein, 17 aa, Artificial Sequence
  Recombinant protein
MIDPSDSFTSYSPSFQG

SEQ ID NO: 566 - protein, 11 aa, Artificial Sequence
  Recombinant protein
LHYYHSEEFDV

SEQ ID NO: 567 - protein, 109 aa, Artificial Sequence; identical to SEQ ID NO: 487
  Recombinant protein

SEQ ID NO: 568 - protein, 12 aa, Artificial Sequence
  Recombinant protein
RASQSVSSSYLA

SEQ ID NO: 569 - protein, 7 aa, Artificial Sequence
  Recombinant protein
GQSSRTR

SEQ ID NO: 570 - protein, 10 aa, Artificial Sequence
  Recombinant protein
QQYGDSQLFT

SEQ ID NO: 571 - protein, 120 aa, Artificial Sequence; identical to SEQ ID NO: 515
  Recombinant protein

SEQ ID NO: 572 - protein, 5 aa, Artificial Sequence
  Recombinant protein
TSWIS

SEQ ID NO: 573 - protein, 17 aa, Artificial Sequence
  Recombinant protein
MIDPSDSYTSYSPSFQG

SEQ ID NO: 574 - protein, 11 aa, Artificial Sequence
  Recombinant protein
LHYYHSEEFDV

SEQ ID NO: 575 - protein, 109 aa, Artificial Sequence; identical to SEQ ID NO: 487
  Recombinant protein

SEQ ID NO: 576 - protein, 12 aa, Artificial Sequence
  Recombinant protein
RASQSVSSSYLA

SEQ ID NO: 577 - protein, 7 aa, Artificial Sequence
  Recombinant protein
GQSSRTR

SEQ ID NO: 578 - protein, 10 aa, Artificial Sequence
  Recombinant protein
QQYGDSQLFT

SEQ ID NO: 579 - protein, 120 aa, Artificial Sequence; identical to SEQ ID NO: 523
  Recombinant protein

SEQ ID NO: 580 - protein, 5 aa, Artificial Sequence
  Recombinant protein
TSWIS

SEQ ID NO: 581 - protein, 17 aa, Artificial Sequence
  Recombinant protein
MIDPSDSYTSYSPSFQG

SEQ ID NO: 582 - protein, 11 aa, Artificial Sequence
  Recombinant protein
LHYYHSEEFDV

SEQ ID NO: 583 - protein, 109 aa, Artificial Sequence; identical to SEQ ID NO: 487
  Recombinant protein

SEQ ID NO: 584 - protein, 12 aa, Artificial Sequence
  Recombinant protein
RASQSVSSSYLA

SEQ ID NO: 585 - protein, 7 aa, Artificial Sequence
  Recombinant protein
GQSSRTR

SEQ ID NO: 586 - protein, 10 aa, Artificial Sequence
  Recombinant protein
QQYGDSQLFT

SEQ ID NO: 587 - protein, 120 aa, Artificial Sequence; identical to SEQ ID NO: 539
  Recombinant protein

SEQ ID NO: 588 - protein, 5 aa, Artificial Sequence
  Recombinant protein
TSWIV

SEQ ID NO: 589 - protein, 17 aa, Artificial Sequence
  Recombinant protein
MIDPSDSYTSYSPSFQG

SEQ ID NO: 590 - protein, 11 aa, Artificial Sequence
  Recombinant protein
LHYYNSEEFDV

SEQ ID NO: 591 - protein, 109 aa, Artificial Sequence; identical to SEQ ID NO: 487
  Recombinant protein

SEQ ID NO: 592 - protein, 12 aa, Artificial Sequence
  Recombinant protein
RASQSVSSSYLA

SEQ ID NO: 593 - protein, 7 aa, Artificial Sequence
  Recombinant protein
GQSSRTR

SEQ ID NO: 594 - protein, 10 aa, Artificial Sequence
  Recombinant protein
QQYGDSQLFT

SEQ ID NO: 595 - protein, 120 aa, Artificial Sequence; identical to SEQ ID NO: 467
  Recombinant protein

SEQ ID NO: 596 - protein, 5 aa, Artificial Sequence
  Recombinant protein
TSWIV

SEQ ID NO: 597 - protein, 17 aa, Artificial Sequence
  Recombinant protein
MIDPSDSFTSYSPSFQG

SEQ ID NO: 598 - protein, 11 aa, Artificial Sequence
  Recombinant protein
LHYYNSEEFDV

SEQ ID NO: 599 - protein, 109 aa, Artificial Sequence
  Recombinant protein
EIVLTQSPGTLSLSPGERATLSCRASQSVSSSYLAWYQQKPGQAPRLLIYGASSRARGIPDRFSGSGSGTDFTLTISRLEPEDFAVYYCQQYGDSRLFTFGQGTKLEIK

SEQ ID NO: 600 - protein, 12 aa, Artificial Sequence
  Recombinant protein
RASQSVSSSYLA

SEQ ID NO: 601 - protein, 7 aa, Artificial Sequence
  Recombinant protein
GASSRAR

SEQ ID NO: 602 - protein, 10 aa, Artificial Sequence
  Recombinant protein
QQYGDSRLFT

SEQ ID NO: 603 - protein, 120 aa, Artificial Sequence; identical to SEQ ID NO: 483
  Recombinant protein

SEQ ID NO: 604 - protein, 5 aa, Artificial Sequence
  Recombinant protein
TSWIS

SEQ ID NO: 605 - protein, 17 aa, Artificial Sequence
  Recombinant protein
MIDPSDSYTSYSPSFQG

SEQ ID NO: 606 - protein, 11 aa, Artificial Sequence
  Recombinant protein
LHYYNSEEFDV

SEQ ID NO: 607 - protein, 109 aa, Artificial Sequence; identical to SEQ ID NO: 599
  Recombinant protein

SEQ ID NO: 608 - protein, 12 aa, Artificial Sequence
  Recombinant protein
RASQSVSSSYLA

SEQ ID NO: 609 - protein, 7 aa, Artificial Sequence
  Recombinant protein
GASSRAR

SEQ ID NO: 610 - protein, 10 aa, Artificial Sequence
  Recombinant protein
QQYGDSRLFT

SEQ ID NO: 611 - protein, 120 aa, Artificial Sequence; identical to SEQ ID NO: 507
  Recombinant protein

SEQ ID NO: 612 - protein, 5 aa, Artificial Sequence
  Recombinant protein
TSWIS

SEQ ID NO: 613 - protein, 17 aa, Artificial Sequence
  Recombinant protein
MIDPSDSFTSYSPSFQG

SEQ ID NO: 614 - protein, 11 aa, Artificial Sequence
  Recombinant protein
LHYYHSEEFDV

SEQ ID NO: 615 - protein, 109 aa, Artificial Sequence; identical to SEQ ID NO: 599
  Recombinant protein

SEQ ID NO: 616 - protein, 12 aa, Artificial Sequence
  Recombinant protein
RASQSVSSSYLA

SEQ ID NO: 617 - protein, 7 aa, Artificial Sequence
  Recombinant protein
GASSRAR

SEQ ID NO: 618 - protein, 10 aa, Artificial Sequence
  Recombinant protein
QQYGDSRLFT

SEQ ID NO: 619 - protein, 120 aa, Artificial Sequence; identical to SEQ ID NO: 515
  Recombinant protein

SEQ ID NO: 620 - protein, 5 aa, Artificial Sequence
  Recombinant protein
TSWIS

SEQ ID NO: 621 - protein, 17 aa, Artificial Sequence
  Recombinant protein
MIDPSDSYTSYSPSFQG

SEQ ID NO: 622 - protein, 11 aa, Artificial Sequence
  Recombinant protein
LHYYHSEEFDV

SEQ ID NO: 623 - protein, 109 aa, Artificial Sequence; identical to SEQ ID NO: 599
  Recombinant protein

SEQ ID NO: 624 - protein, 12 aa, Artificial Sequence
  Recombinant protein
RASQSVSSSYLA

SEQ ID NO: 625 - protein, 7 aa, Artificial Sequence
  Recombinant protein
GASSRAR

SEQ ID NO: 626 - protein, 10 aa, Artificial Sequence
  Recombinant protein
QQYGDSRLFT

SEQ ID NO: 627 - protein, 120 aa, Artificial Sequence; identical to SEQ ID NO: 523
  Recombinant protein

SEQ ID NO: 628 - protein, 5 aa, Artificial Sequence
  Recombinant protein
TSWIS

SEQ ID NO: 629 - protein, 17 aa, Artificial Sequence
  Recombinant protein
MIDPSDSYTSYSPSFQG

SEQ ID NO: 630 - protein, 11 aa, Artificial Sequence
  Recombinant protein
LHYYHSEEFDV

SEQ ID NO: 631 - protein, 109 aa, Artificial Sequence; identical to SEQ ID NO: 599
  Recombinant protein

SEQ ID NO: 632 - protein, 12 aa, Artificial Sequence
  Recombinant protein
RASQSVSSSYLA

SEQ ID NO: 633 - protein, 7 aa, Artificial Sequence
  Recombinant protein
GASSRAR

SEQ ID NO: 634 - protein, 10 aa, Artificial Sequence
  Recombinant protein
QQYGDSRLFT

SEQ ID NO: 635 - protein, 120 aa, Artificial Sequence; identical to SEQ ID NO: 491
  Recombinant protein

SEQ ID NO: 636 - protein, 5 aa, Artificial Sequence
  Recombinant protein
TSWIS

SEQ ID NO: 637 - protein, 17 aa, Artificial Sequence
  Recombinant protein
MIDPSDSFTSYSPSFQG

SEQ ID NO: 638 - protein, 11 aa, Artificial Sequence
  Recombinant protein
LHYYNSEEFDV

SEQ ID NO: 639 - protein, 109 aa, Artificial Sequence; identical to SEQ ID NO: 599
  Recombinant protein

SEQ ID NO: 640 - protein, 12 aa, Artificial Sequence
  Recombinant protein
RASQSVSSSYLA

SEQ ID NO: 641 - protein, 7 aa, Artificial Sequence
  Recombinant protein
GASSRAR

SEQ ID NO: 642 - protein, 10 aa, Artificial Sequence
  Recombinant protein
QQYGDSRLFT

SEQ ID NO: 643 - protein, 120 aa, Artificial Sequence; identical to SEQ ID NO: 539
  Recombinant protein

SEQ ID NO: 644 - protein, 5 aa, Artificial Sequence
  Recombinant protein
TSWIV

SEQ ID NO: 645 - protein, 17 aa, Artificial Sequence
  Recombinant protein
MIDPSDSYTSYSPSFQG

SEQ ID NO: 646 - protein, 11 aa, Artificial Sequence
  Recombinant protein
LHYYNSEEFDV

SEQ ID NO: 647 - protein, 109 aa, Artificial Sequence; identical to SEQ ID NO: 599
  Recombinant protein

SEQ ID NO: 648 - protein, 12 aa, Artificial Sequence
  Recombinant protein
RASQSVSSSYLA

SEQ ID NO: 649 - protein, 7 aa, Artificial Sequence
  Recombinant protein
GASSRAR

SEQ ID NO: 650 - protein, 10 aa, Artificial Sequence
  Recombinant protein
QQYGDSRLFT

SEQ ID NO: 651 - protein, 120 aa, Artificial Sequence; identical to SEQ ID NO: 467
  Recombinant protein

SEQ ID NO: 652 - protein, 5 aa, Artificial Sequence
  Recombinant protein
TSWIV

SEQ ID NO: 653 - protein, 17 aa, Artificial Sequence
  Recombinant protein
MIDPSDSFTSYSPSFQG

SEQ ID NO: 654 - protein, 11 aa, Artificial Sequence
  Recombinant protein
LHYYNSEEFDV

SEQ ID NO: 655 - protein, 109 aa, Artificial Sequence
  Recombinant protein
EIVLTQSPGTLSLSPGERATLSCRASQSVSSSYLAWYQQKPGQAPRLLIYGQSSRTRGIPDRFSGSGSGTDFTLTISRLEPEDFAVYYCQQFGDSRLFTFGQGTKLEIK

SEQ ID NO: 656 - protein, 12 aa, Artificial Sequence
  Recombinant protein
RASQSVSSSYLA

SEQ ID NO: 657 - protein, 7 aa, Artificial Sequence
  Recombinant protein
GQSSRTR

SEQ ID NO: 658 - protein, 10 aa, Artificial Sequence
  Recombinant protein
QQFGDSRLFT

SEQ ID NO: 659 - protein, 120 aa, Artificial Sequence; identical to SEQ ID NO: 483
  Recombinant protein

SEQ ID NO: 660 - protein, 5 aa, Artificial Sequence
  Recombinant protein
TSWIS

SEQ ID NO: 661 - protein, 17 aa, Artificial Sequence
  Recombinant protein
MIDPSDSYTSYSPSFQG

SEQ ID NO: 662 - protein, 11 aa, Artificial Sequence
  Recombinant protein
LHYYNSEEFDV

SEQ ID NO: 663 - protein, 109 aa, Artificial Sequence; identical to SEQ ID NO: 655
  Recombinant protein

SEQ ID NO: 664 - protein, 12 aa, Artificial Sequence
  Recombinant protein
RASQSVSSSYLA

SEQ ID NO: 665 - protein, 7 aa, Artificial Sequence
  Recombinant protein
GQSSRTR

SEQ ID NO: 666 - protein, 10 aa, Artificial Sequence
  Recombinant protein
QQFGDSRLFT

SEQ ID NO: 667 - protein, 120 aa, Artificial Sequence; identical to SEQ ID NO: 507
  Recombinant protein

SEQ ID NO: 668 - protein, 5 aa, Artificial Sequence
  Recombinant protein
TSWIS

SEQ ID NO: 669 - protein, 17 aa, Artificial Sequence
  Recombinant protein
MIDPSDSFTSYSPSFQG

SEQ ID NO: 670 - protein, 11 aa, Artificial Sequence
  Recombinant protein
LHYYHSEEFDV

SEQ ID NO: 671 - protein, 109 aa, Artificial Sequence; identical to SEQ ID NO: 655
  Recombinant protein

SEQ ID NO: 672 - protein, 12 aa, Artificial Sequence
  Recombinant protein
RASQSVSSSYLA

SEQ ID NO: 673 - protein, 7 aa, Artificial Sequence
  Recombinant protein
GQSSRTR

SEQ ID NO: 674 - protein, 10 aa, Artificial Sequence
  Recombinant protein
QQFGDSRLFT

SEQ ID NO: 675 - protein, 120 aa, Artificial Sequence; identical to SEQ ID NO: 515
  Recombinant protein

SEQ ID NO: 676 - protein, 5 aa, Artificial Sequence
  Recombinant protein
TSWIS

SEQ ID NO: 677 - protein, 17 aa, Artificial Sequence
  Recombinant protein
MIDPSDSYTSYSPSFQG

SEQ ID NO: 678 - protein, 11 aa, Artificial Sequence
  Recombinant protein
LHYYHSEEFDV

SEQ ID NO: 679 - protein, 109 aa, Artificial Sequence; identical to SEQ ID NO: 655
  Recombinant protein

SEQ ID NO: 680 - protein, 12 aa, Artificial Sequence
  Recombinant protein
RASQSVSSSYLA

SEQ ID NO: 681 - protein, 7 aa, Artificial Sequence
  Recombinant protein
GQSSRTR

SEQ ID NO: 682 - protein, 10 aa, Artificial Sequence
  Recombinant protein
QQFGDSRLFT

SEQ ID NO: 683 - protein, 120 aa, Artificial Sequence; identical to SEQ ID NO: 523
  Recombinant protein

SEQ ID NO: 684 - protein, 5 aa, Artificial Sequence
  Recombinant protein
TSWIS

SEQ ID NO: 685 - protein, 17 aa, Artificial Sequence
  Recombinant protein
MIDPSDSYTSYSPSFQG

SEQ ID NO: 686 - protein, 11 aa, Artificial Sequence
  Recombinant protein
LHYYHSEEFDV

SEQ ID NO: 687 - protein, 109 aa, Artificial Sequence; identical to SEQ ID NO: 655
  Recombinant protein

SEQ ID NO: 688 - protein, 12 aa, Artificial Sequence
  Recombinant protein
RASQSVSSSYLA

SEQ ID NO: 689 - protein, 7 aa, Artificial Sequence
  Recombinant protein
GQSSRTR

SEQ ID NO: 690 - protein, 10 aa, Artificial Sequence
  Recombinant protein
QQFGDSRLFT

SEQ ID NO: 691 - protein, 120 aa, Artificial Sequence; identical to SEQ ID NO: 491
  Recombinant protein

SEQ ID NO: 692 - protein, 5 aa, Artificial Sequence
  Recombinant protein
TSWIS

SEQ ID NO: 693 - protein, 17 aa, Artificial Sequence
  Recombinant protein
MIDPSDSFTSYSPSFQG

SEQ ID NO: 694 - protein, 11 aa, Artificial Sequence
  Recombinant protein
LHYYNSEEFDV

SEQ ID NO: 695 - protein, 109 aa, Artificial Sequence; identical to SEQ ID NO: 655
  Recombinant protein

SEQ ID NO: 696 - protein, 12 aa, Artificial Sequence
  Recombinant protein
RASQSVSSSYLA

SEQ ID NO: 697 - protein, 7 aa, Artificial Sequence
  Recombinant protein
GQSSRTR

SEQ ID NO: 698 - protein, 10 aa, Artificial Sequence
  Recombinant protein
QQFGDSRLFT

SEQ ID NO: 699 - protein, 120 aa, Artificial Sequence; identical to SEQ ID NO: 539
  Recombinant protein

SEQ ID NO: 700 - protein, 5 aa, Artificial Sequence
  Recombinant protein
TSWIV

SEQ ID NO: 701 - protein, 17 aa, Artificial Sequence
  Recombinant protein
MIDPSDSYTSYSPSFQG

SEQ ID NO: 702 - protein, 11 aa, Artificial Sequence
  Recombinant protein
LHYYNSEEFDV

SEQ ID NO: 703 - protein, 109 aa, Artificial Sequence; identical to SEQ ID NO: 655
  Recombinant protein

SEQ ID NO: 704 - protein, 12 aa, Artificial Sequence
  Recombinant protein
RASQSVSSSYLA

SEQ ID NO: 705 - protein, 7 aa, Artificial Sequence
  Recombinant protein
GQSSRTR

SEQ ID NO: 706 - protein, 10 aa, Artificial Sequence
  Recombinant protein
QQFGDSRLFT

SEQ ID NO: 707 - protein, 120 aa, Artificial Sequence; identical to SEQ ID NO: 467
  Recombinant protein

SEQ ID NO: 708 - protein, 5 aa, Artificial Sequence
  Recombinant protein
TSWIV

SEQ ID NO: 709 - protein, 17 aa, Artificial Sequence
  Recombinant protein
MIDPSDSFTSYSPSFQG

SEQ ID NO: 710 - protein, 11 aa, Artificial Sequence
  Recombinant protein
LHYYNSEEFDV

SEQ ID NO: 711 - protein, 109 aa, Artificial Sequence
  Recombinant protein
EIVLTQSPGTLSLSPGERATLSCRASQSVSSSYLAWYQQKPGQAPRLLIYGQSSRTRGIPDRFSGSGSGTDFTLTISRLEPEDFAVYYCQQFGSSQLFTFGQGTKLEIK

SEQ ID NO: 712 - protein, 12 aa, Artificial Sequence
  Recombinant protein
RASQSVSSSYLA

SEQ ID NO: 713 - protein, 7 aa, Artificial Sequence
  Recombinant protein
GQSSRTR

SEQ ID NO: 714 - protein, 10 aa, Artificial Sequence
  Recombinant protein
QQFGSSQLFT

SEQ ID NO: 715 - protein, 120 aa, Artificial Sequence; identical to SEQ ID NO: 467
  Recombinant protein

SEQ ID NO: 716 - protein, 5 aa, Artificial Sequence
  Recombinant protein
TSWIV

SEQ ID NO: 717 - protein, 17 aa, Artificial Sequence
  Recombinant protein
MIDPSDSFTSYSPSFQG

SEQ ID NO: 718 - protein, 11 aa, Artificial Sequence
  Recombinant protein
LHYYNSEEFDV

SEQ ID NO: 719 - protein, 109 aa, Artificial Sequence
  Recombinant protein
EIVLTQSPGTLSLSPGERATLSCRASQSVSSSYLAWYQQKPGQAPRLLIYGQSSRTRGIPDRFSGSGSGTDFTLTISRLEPEDFAVYYCQQFGESQLFTFGQGTKLEIK

SEQ ID NO: 720 - protein, 12 aa, Artificial Sequence
  Recombinant protein
RASQSVSSSYLA

SEQ ID NO: 721 - protein, 7 aa, Artificial Sequence
  Recombinant protein
GQSSRTR

SEQ ID NO: 722 - protein, 10 aa, Artificial Sequence
  Recombinant protein
QQFGESQLFT

SEQ ID NO: 723 - protein, 120 aa, Artificial Sequence; identical to SEQ ID NO: 467
  Recombinant protein

SEQ ID NO: 724 - protein, 5 aa, Artificial Sequence
  Recombinant protein
TSWIV

SEQ ID NO: 725 - protein, 17 aa, Artificial Sequence
  Recombinant protein
MIDPSDSFTSYSPSFQG

SEQ ID NO: 726 - protein, 11 aa, Artificial Sequence
  Recombinant protein
LHYYNSEEFDV

SEQ ID NO: 727 - protein, 109 aa, Artificial Sequence
  Recombinant protein
EIVLTQSPGTLSLSPGERATLSCRASQSVSSSYLAWYQQKPGQAPRLLIYGQSSRTRGIPDRFSGSGSGTDFTLTISRLEPEDFAVYYCQQFGNSQLFTFGQGTKLEIK

SEQ ID NO: 728 - protein, 12 aa, Artificial Sequence
  Recombinant protein
RASQSVSSSYLA

SEQ ID NO: 729 - protein, 7 aa, Artificial Sequence
  Recombinant protein
GQSSRTR

SEQ ID NO: 730 - protein, 10 aa, Artificial Sequence
  Recombinant protein
QQFGNSQLFT

SEQ ID NO: 731 - protein, 120 aa, Artificial Sequence; identical to SEQ ID NO: 467
  Recombinant protein

SEQ ID NO: 732 - protein, 5 aa, Artificial Sequence
  Recombinant protein
TSWIV

SEQ ID NO: 733 - protein, 17 aa, Artificial Sequence
  Recombinant protein
MIDPSDSFTSYSPSFQG

SEQ ID NO: 734 - protein, 11 aa, Artificial Sequence
  Recombinant protein
LHYYNSEEFDV

SEQ ID NO: 735 - protein, 109 aa, Artificial Sequence
  Recombinant protein
EIVLTQSPGTLSLSPGERATLSCRASQSVSSSYLAWYQQKPGQAPRLLIYGQSSRTRGIPDRFSGSGSGTDFTLTISRLEPEDFAVYYCQQFGQSQLFTFGQGTKLEIK

SEQ ID NO: 736 - protein, 12 aa, Artificial Sequence
  Recombinant protein
RASQSVSSSYLA

SEQ ID NO: 737 - protein, 7 aa, Artificial Sequence
  Recombinant protein
GQSSRTR

SEQ ID NO: 738 - protein, 10 aa, Artificial Sequence
  Recombinant protein
QQFGQSQLFT

SEQ ID NO: 739 - protein, 120 aa, Artificial Sequence; identical to SEQ ID NO: 467
  Recombinant protein

SEQ ID NO: 740 - protein, 5 aa, Artificial Sequence
  Recombinant protein
TSWIV

SEQ ID NO: 741 - protein, 17 aa, Artificial Sequence
  Recombinant protein
MIDPSDSFTSYSPSFQG

SEQ ID NO: 742 - protein, 11 aa, Artificial Sequence
  Recombinant protein
LHYYNSEEFDV

SEQ ID NO: 743 - protein, 109 aa, Artificial Sequence
  Recombinant protein
EIVLTQSPGTLSLSPGERATLSCRASQSVSSSYLAWYQQKPGQAPRLLIYGQSSRTRGIPDRFSGSGSGTDFTLTISRLEPEDFAVYYCQQFGDAQLFTFGQGTKLEIK

SEQ ID NO: 744 - protein, 12 aa, Artificial Sequence
  Recombinant protein
RASQSVSSSYLA

SEQ ID NO: 745 - protein, 7 aa, Artificial Sequence
  Recombinant protein
GQSSRTR

SEQ ID NO: 746 - protein, 10 aa, Artificial Sequence
  Recombinant protein
QQFGDAQLFT

SEQ ID NO: 747 - protein, 120 aa, Artificial Sequence; identical to SEQ ID NO: 467
  Recombinant protein

SEQ ID NO: 748 - protein, 5 aa, Artificial Sequence
  Recombinant protein
TSWIV

SEQ ID NO: 749 - protein, 17 aa, Artificial Sequence
  Recombinant protein
MIDPSDSFTSYSPSFQG

SEQ ID NO: 750 - protein, 11 aa, Artificial Sequence
  Recombinant protein
LHYYNSEEFDV

SEQ ID NO: 751 - protein, 109 aa, Artificial Sequence
  Recombinant protein
EIVLTQSPGTLSLSPGERATLSCRASQSVSSSYLAWYQQKPGQAPRLLIYGQSSRTRGIPDRFSGSGSGTDFTLTISRLEPEDFAVYYCQQFGDTQLFTFGQGTKLEIK

SEQ ID NO: 752 - protein, 12 aa, Artificial Sequence
  Recombinant protein
RASQSVSSSYLA

SEQ ID NO: 753 - protein, 7 aa, Artificial Sequence
  Recombinant protein
GQSSRTR

SEQ ID NO: 754 - protein, 10 aa, Artificial Sequence
  Recombinant protein
QQFGDTQLFT

SEQ ID NO: 755 - protein, 120 aa, Artificial Sequence; identical to SEQ ID NO: 467
  Recombinant protein

SEQ ID NO: 756 - protein, 5 aa, Artificial Sequence
  Recombinant protein
TSWIV

SEQ ID NO: 757 - protein, 17 aa, Artificial Sequence
  Recombinant protein
MIDPSDSFTSYSPSFQG

SEQ ID NO: 758 - protein, 11 aa, Artificial Sequence
  Recombinant protein
LHYYNSEEFDV

SEQ ID NO: 759 - protein, 109 aa, Artificial Sequence
  Recombinant protein
EIVLTQSPGTLSLSPGERATLSCRASQSVSSSYLAWYQQKPGQAPRLLIYGQSSRTRGIPDRFSGSGSGTDFTLTISRLEPEDFAVYYCQQFGDNQLFTFGQGTKLEIK

SEQ ID NO: 760 - protein, 12 aa, Artificial Sequence
  Recombinant protein
RASQSVSSSYLA

SEQ ID NO: 761 - protein, 7 aa, Artificial Sequence
  Recombinant protein
GQSSRTR

SEQ ID NO: 762 - protein, 10 aa, Artificial Sequence
  Recombinant protein
QQFGDNQLFT

SEQ ID NO: 763 - protein, 120 aa, Artificial Sequence; identical to SEQ ID NO: 483
  Recombinant protein

SEQ ID NO: 764 - protein, 5 aa, Artificial Sequence
  Recombinant protein
TSWIS

SEQ ID NO: 765 - protein, 17 aa, Artificial Sequence
  Recombinant protein
MIDPSDSYTSYSPSFQG

SEQ ID NO: 766 - protein, 11 aa, Artificial Sequence
  Recombinant protein
LHYYNSEEFDV

SEQ ID NO: 767 - protein, 109 aa, Artificial Sequence; identical to SEQ ID NO: 711
  Recombinant protein

SEQ ID NO: 768 - protein, 12 aa, Artificial Sequence
  Recombinant protein
RASQSVSSSYLA

SEQ ID NO: 769 - protein, 7 aa, Artificial Sequence
  Recombinant protein
GQSSRTR

SEQ ID NO: 770 - protein, 10 aa, Artificial Sequence
  Recombinant protein
QQFGSSQLFT

SEQ ID NO: 771 - protein, 120 aa, Artificial Sequence; identical to SEQ ID NO: 507
  Recombinant protein

SEQ ID NO: 772 - protein, 5 aa, Artificial Sequence
  Recombinant protein
TSWIS

SEQ ID NO: 773 - protein, 17 aa, Artificial Sequence
  Recombinant protein
MIDPSDSFTSYSPSFQG

SEQ ID NO: 774 - protein, 11 aa, Artificial Sequence
  Recombinant protein
LHYYHSEEFDV

SEQ ID NO: 775 - protein, 109 aa, Artificial Sequence; identical to SEQ ID NO: 711
  Recombinant protein

SEQ ID NO: 776 - protein, 12 aa, Artificial Sequence
  Recombinant protein
RASQSVSSSYLA

SEQ ID NO: 777 - protein, 7 aa, Artificial Sequence
  Recombinant protein
GQSSRTR

SEQ ID NO: 778 - protein, 10 aa, Artificial Sequence
  Recombinant protein
QQFGSSQLFT

SEQ ID NO: 779 - protein, 120 aa, Artificial Sequence; identical to SEQ ID NO: 515
  Recombinant protein

SEQ ID NO: 780 - protein, 5 aa, Artificial Sequence
  Recombinant protein
TSWIS

SEQ ID NO: 781 - protein, 17 aa, Artificial Sequence
  Recombinant protein
MIDPSDSYTSYSPSFQG

SEQ ID NO: 782 - protein, 11 aa, Artificial Sequence
  Recombinant protein
LHYYHSEEFDV

SEQ ID NO: 783 - protein, 109 aa, Artificial Sequence; identical to SEQ ID NO: 711
  Recombinant protein

SEQ ID NO: 784 - protein, 12 aa, Artificial Sequence
  Recombinant protein
RASQSVSSSYLA

SEQ ID NO: 785 - protein, 7 aa, Artificial Sequence
  Recombinant protein
GQSSRTR

SEQ ID NO: 786 - protein, 10 aa, Artificial Sequence
  Recombinant protein
QQFGSSQLFT

SEQ ID NO: 787 - protein, 120 aa, Artificial Sequence; identical to SEQ ID NO: 523
  Recombinant protein

SEQ ID NO: 788 - protein, 5 aa, Artificial Sequence
  Recombinant protein
TSWIS

SEQ ID NO: 789 - protein, 17 aa, Artificial Sequence
  Recombinant protein
MIDPSDSYTSYSPSFQG

SEQ ID NO: 790 - protein, 11 aa, Artificial Sequence
  Recombinant protein
LHYYHSEEFDV

SEQ ID NO: 791 - protein, 109 aa, Artificial Sequence; identical to SEQ ID NO: 711
  Recombinant protein

SEQ ID NO: 792 - protein, 12 aa, Artificial Sequence
  Recombinant protein
RASQSVSSSYLA

SEQ ID NO: 793 - protein, 7 aa, Artificial Sequence
  Recombinant protein
GQSSRTR

SEQ ID NO: 794 - protein, 10 aa, Artificial Sequence
  Recombinant protein
QQFGSSQLFT

SEQ ID NO: 795 - protein, 120 aa, Artificial Sequence; identical to SEQ ID NO: 491
  Recombinant protein

SEQ ID NO: 796 - protein, 5 aa, Artificial Sequence
  Recombinant protein
TSWIS

SEQ ID NO: 797 - protein, 17 aa, Artificial Sequence
  Recombinant protein
MIDPSDSFTSYSPSFQG

SEQ ID NO: 798 - protein, 11 aa, Artificial Sequence
  Recombinant protein
LHYYNSEEFDV

SEQ ID NO: 799 - protein, 109 aa, Artificial Sequence; identical to SEQ ID NO: 711
  Recombinant protein

SEQ ID NO: 800 - protein, 12 aa, Artificial Sequence
  Recombinant protein
RASQSVSSSYLA

SEQ ID NO: 801 - protein, 7 aa, Artificial Sequence
  Recombinant protein
GQSSRTR

SEQ ID NO: 802 - protein, 10 aa, Artificial Sequence
  Recombinant protein
QQFGSSQLFT

SEQ ID NO: 803 - protein, 120 aa, Artificial Sequence; identical to SEQ ID NO: 539
  Recombinant protein

SEQ ID NO: 804 - protein, 5 aa, Artificial Sequence
  Recombinant protein
TSWIV

SEQ ID NO: 805 - protein, 17 aa, Artificial Sequence
  Recombinant protein
MIDPSDSYTSYSPSFQG

SEQ ID NO: 806 - protein, 11 aa, Artificial Sequence
  Recombinant protein
LHYYNSEEFDV

SEQ ID NO: 807 - protein, 109 aa, Artificial Sequence; identical to SEQ ID NO: 711
  Recombinant protein

SEQ ID NO: 808 - protein, 12 aa, Artificial Sequence
  Recombinant protein
RASQSVSSSYLA

SEQ ID NO: 809 - protein, 7 aa, Artificial Sequence
  Recombinant protein
GQSSRTR

SEQ ID NO: 810 - protein, 10 aa, Artificial Sequence
  Recombinant protein
QQFGSSQLFT

SEQ ID NO: 811 - protein, 120 aa, Artificial Sequence; identical to SEQ ID NO: 547
  Recombinant protein

SEQ ID NO: 812 - protein, 5 aa, Artificial Sequence
  Recombinant protein
TSWIV

SEQ ID NO: 813 - protein, 17 aa, Artificial Sequence
  Recombinant protein
MIDPSDSYTSYSPSFQG

SEQ ID NO: 814 - protein, 11 aa, Artificial Sequence
  Recombinant protein
LHYYNSEEFDV

SEQ ID NO: 815 - protein, 109 aa, Artificial Sequence; identical to SEQ ID NO: 711
  Recombinant protein

SEQ ID NO: 816 - protein, 12 aa, Artificial Sequence
  Recombinant protein
RASQSVSSSYLA

SEQ ID NO: 817 - protein, 7 aa, Artificial Sequence
  Recombinant protein
GQSSRTR

SEQ ID NO: 818 - protein, 10 aa, Artificial Sequence
  Recombinant protein
QQFGSSQLFT

SEQ ID NO: 819 - protein, 120 aa, Artificial Sequence; identical to SEQ ID NO: 483
  Recombinant protein

SEQ ID NO: 820 - protein, 5 aa, Artificial Sequence
  Recombinant protein
TSWIS

SEQ ID NO: 821 - protein, 17 aa, Artificial Sequence
  Recombinant protein
MIDPSDSYTSYSPSFQG

SEQ ID NO: 822 - protein, 11 aa, Artificial Sequence
  Recombinant protein
LHYYNSEEFDV

SEQ ID NO: 823 - protein, 109 aa, Artificial Sequence; identical to SEQ ID NO: 719
  Recombinant protein

SEQ ID NO: 824 - protein, 12 aa, Artificial Sequence
  Recombinant protein
RASQSVSSSYLA

SEQ ID NO: 825 - protein, 7 aa, Artificial Sequence
  Recombinant protein
GQSSRTR

SEQ ID NO: 826 - protein, 10 aa, Artificial Sequence
  Recombinant protein
QQFGESQLFT

SEQ ID NO: 827 - protein, 120 aa, Artificial Sequence; identical to SEQ ID NO: 507
  Recombinant protein

SEQ ID NO: 828 - protein, 5 aa, Artificial Sequence
  Recombinant protein
TSWIS

SEQ ID NO: 829 - protein, 17 aa, Artificial Sequence
  Recombinant protein
MIDPSDSFTSYSPSFQG

SEQ ID NO: 830 - protein, 11 aa, Artificial Sequence
  Recombinant protein
LHYYHSEEFDV

SEQ ID NO: 831 - protein, 109 aa, Artificial Sequence; identical to SEQ ID NO: 719
  Recombinant protein

SEQ ID NO: 832 - protein, 12 aa, Artificial Sequence
  Recombinant protein
RASQSVSSSYLA

SEQ ID NO: 833 - protein, 7 aa, Artificial Sequence
  Recombinant protein
GQSSRTR

SEQ ID NO: 834 - protein, 10 aa, Artificial Sequence
  Recombinant protein
QQFGESQLFT

SEQ ID NO: 835 - protein, 120 aa, Artificial Sequence; identical to SEQ ID NO: 515
  Recombinant protein

SEQ ID NO: 836 - protein, 5 aa, Artificial Sequence
  Recombinant protein
TSWIS

SEQ ID NO: 837 - protein, 17 aa, Artificial Sequence
  Recombinant protein
MIDPSDSYTSYSPSFQG

SEQ ID NO: 838 - protein, 11 aa, Artificial Sequence
  Recombinant protein
LHYYHSEEFDV

SEQ ID NO: 839 - protein, 109 aa, Artificial Sequence; identical to SEQ ID NO: 719
  Recombinant protein

SEQ ID NO: 840 - protein, 12 aa, Artificial Sequence
  Recombinant protein
RASQSVSSSYLA

SEQ ID NO: 841 - protein, 7 aa, Artificial Sequence
  Recombinant protein
GQSSRTR

SEQ ID NO: 842 - protein, 10 aa, Artificial Sequence
  Recombinant protein
QQFGESQLFT

SEQ ID NO: 843 - protein, 120 aa, Artificial Sequence; identical to SEQ ID NO: 523
  Recombinant protein

SEQ ID NO: 844 - protein, 5 aa, Artificial Sequence
  Recombinant protein
TSWIS

SEQ ID NO: 845 - protein, 17 aa, Artificial Sequence
  Recombinant protein
MIDPSDSYTSYSPSFQG

SEQ ID NO: 846 - protein, 11 aa, Artificial Sequence
  Recombinant protein
LHYYHSEEFDV

SEQ ID NO: 847 - protein, 109 aa, Artificial Sequence; identical to SEQ ID NO: 719
  Recombinant protein

SEQ ID NO: 848 - protein, 12 aa, Artificial Sequence
  Recombinant protein
RASQSVSSSYLA

SEQ ID NO: 849 - protein, 7 aa, Artificial Sequence
  Recombinant protein
GQSSRTR

SEQ ID NO: 850 - protein, 10 aa, Artificial Sequence
  Recombinant protein
QQFGESQLFT

SEQ ID NO: 851 - protein, 120 aa, Artificial Sequence; identical to SEQ ID NO: 491
  Recombinant protein

SEQ ID NO: 852 - protein, 5 aa, Artificial Sequence
  Recombinant protein
TSWIS

SEQ ID NO: 853 - protein, 17 aa, Artificial Sequence
  Recombinant protein
MIDPSDSFTSYSPSFQG

SEQ ID NO: 854 - protein, 11 aa, Artificial Sequence
  Recombinant protein
LHYYNSEEFDV

SEQ ID NO: 855 - protein, 109 aa, Artificial Sequence; identical to SEQ ID NO: 719
  Recombinant protein

SEQ ID NO: 856 - protein, 12 aa, Artificial Sequence
  Recombinant protein
RASQSVSSSYLA

SEQ ID NO: 857 - protein, 7 aa, Artificial Sequence
  Recombinant protein
GQSSRTR

SEQ ID NO: 858 - protein, 10 aa, Artificial Sequence
  Recombinant protein
QQFGESQLFT

SEQ ID NO: 859 - protein, 120 aa, Artificial Sequence; identical to SEQ ID NO: 539
  Recombinant protein

SEQ ID NO: 860 - protein, 5 aa, Artificial Sequence
  Recombinant protein
TSWIV

SEQ ID NO: 861 - protein, 17 aa, Artificial Sequence
  Recombinant protein
MIDPSDSYTSYSPSFQG

SEQ ID NO: 862 - protein, 11 aa, Artificial Sequence
  Recombinant protein
LHYYNSEEFDV

SEQ ID NO: 863 - protein, 109 aa, Artificial Sequence; identical to SEQ ID NO: 719
  Recombinant protein

SEQ ID NO: 864 - protein, 12 aa, Artificial Sequence
  Recombinant protein
RASQSVSSSYLA

SEQ ID NO: 865 - protein, 7 aa, Artificial Sequence
  Recombinant protein
GQSSRTR

SEQ ID NO: 866 - protein, 10 aa, Artificial Sequence
  Recombinant protein
QQFGESQLFT

SEQ ID NO: 867 - protein, 120 aa, Artificial Sequence; identical to SEQ ID NO: 547
  Recombinant protein

SEQ ID NO: 868 - protein, 5 aa, Artificial Sequence
  Recombinant protein
TSWIV

SEQ ID NO: 869 - protein, 17 aa, Artificial Sequence
  Recombinant protein
MIDPSDSYTSYSPSFQG

SEQ ID NO: 870 - protein, 11 aa, Artificial Sequence
  Recombinant protein
LHYYNSEEFDV

SEQ ID NO: 871 - protein, 109 aa, Artificial Sequence; identical to SEQ ID NO: 719
  Recombinant protein

SEQ ID NO: 872 - protein, 12 aa, Artificial Sequence
  Recombinant protein
RASQSVSSSYLA

SEQ ID NO: 873 - protein, 7 aa, Artificial Sequence
  Recombinant protein
GQSSRTR

SEQ ID NO: 874 - protein, 10 aa, Artificial Sequence
  Recombinant protein
QQFGESQLFT

SEQ ID NO: 875 - protein, 120 aa, Artificial Sequence; identical to SEQ ID NO: 483
  Recombinant protein

SEQ ID NO: 876 - protein, 5 aa, Artificial Sequence
  Recombinant protein
TSWIS

SEQ ID NO: 877 - protein, 17 aa, Artificial Sequence
  Recombinant protein
MIDPSDSYTSYSPSFQG

SEQ ID NO: 878 - protein, 11 aa, Artificial Sequence
  Recombinant protein
LHYYNSEEFDV

SEQ ID NO: 879 - protein, 109 aa, Artificial Sequence; identical to SEQ ID NO: 727
  Recombinant protein

SEQ ID NO: 880 - protein, 12 aa, Artificial Sequence
  Recombinant protein
RASQSVSSSYLA

SEQ ID NO: 881 - protein, 7 aa, Artificial Sequence
  Recombinant protein
GQSSRTR

SEQ ID NO: 882 - protein, 10 aa, Artificial Sequence
  Recombinant protein
QQFGNSQLFT

SEQ ID NO: 883 - protein, 120 aa, Artificial Sequence; identical to SEQ ID NO: 507
  Recombinant protein

SEQ ID NO: 884 - protein, 5 aa, Artificial Sequence
  Recombinant protein
TSWIS

SEQ ID NO: 885 - protein, 17 aa, Artificial Sequence
  Recombinant protein
MIDPSDSFTSYSPSFQG

SEQ ID NO: 886 - protein, 11 aa, Artificial Sequence
  Recombinant protein
LHYYHSEEFDV

SEQ ID NO: 887 - protein, 109 aa, Artificial Sequence; identical to SEQ ID NO: 727
  Recombinant protein

SEQ ID NO: 888 - protein, 12 aa, Artificial Sequence
  Recombinant protein
RASQSVSSSYLA

SEQ ID NO: 889 - protein, 7 aa, Artificial Sequence
  Recombinant protein
GQSSRTR

SEQ ID NO: 890 - protein, 10 aa, Artificial Sequence
  Recombinant protein
QQFGNSQLFT

SEQ ID NO: 891 - protein, 120 aa, Artificial Sequence; identical to SEQ ID NO: 515
  Recombinant protein

SEQ ID NO: 892 - protein, 5 aa, Artificial Sequence
  Recombinant protein
TSWIS

SEQ ID NO: 893 - protein, 17 aa, Artificial Sequence
  Recombinant protein
MIDPSDSYTSYSPSFQG

SEQ ID NO: 894 - protein, 11 aa, Artificial Sequence
  Recombinant protein
LHYYHSEEFDV

SEQ ID NO: 895 - protein, 109 aa, Artificial Sequence; identical to SEQ ID NO: 727
  Recombinant protein

SEQ ID NO: 896 - protein, 12 aa, Artificial Sequence
  Recombinant protein
RASQSVSSSYLA

SEQ ID NO: 897 - protein, 7 aa, Artificial Sequence
  Recombinant protein
GQSSRTR

SEQ ID NO: 898 - protein, 10 aa, Artificial Sequence
  Recombinant protein
QQFGNSQLFT

SEQ ID NO: 899 - protein, 120 aa, Artificial Sequence; identical to SEQ ID NO: 523
  Recombinant protein

SEQ ID NO: 900 - protein, 5 aa, Artificial Sequence
  Recombinant protein
TSWIS

SEQ ID NO: 901 - protein, 17 aa, Artificial Sequence
  Recombinant protein
MIDPSDSYTSYSPSFQG

SEQ ID NO: 902 - protein, 11 aa, Artificial Sequence
  Recombinant protein
LHYYHSEEFDV

SEQ ID NO: 903 - protein, 109 aa, Artificial Sequence; identical to SEQ ID NO: 727
  Recombinant protein

SEQ ID NO: 904 - protein, 12 aa, Artificial Sequence
  Recombinant protein
RASQSVSSSYLA

SEQ ID NO: 905 - protein, 7 aa, Artificial Sequence
  Recombinant protein
GQSSRTR

SEQ ID NO: 906 - protein, 10 aa, Artificial Sequence
  Recombinant protein
QQFGNSQLFT

SEQ ID NO: 907 - protein, 120 aa, Artificial Sequence; identical to SEQ ID NO: 491
  Recombinant protein

SEQ ID NO: 908 - protein, 5 aa, Artificial Sequence
  Recombinant protein
TSWIS

SEQ ID NO: 909 - protein, 17 aa, Artificial Sequence
  Recombinant protein
MIDPSDSFTSYSPSFQG

SEQ ID NO: 910 - protein, 11 aa, Artificial Sequence
  Recombinant protein
LHYYNSEEFDV

SEQ ID NO: 911 - protein, 109 aa, Artificial Sequence; identical to SEQ ID NO: 727
  Recombinant protein

SEQ ID NO: 912 - protein, 12 aa, Artificial Sequence
  Recombinant protein
RASQSVSSSYLA

SEQ ID NO: 913 - protein, 7 aa, Artificial Sequence
  Recombinant protein
GQSSRTR

SEQ ID NO: 914 - protein, 10 aa, Artificial Sequence
  Recombinant protein
QQFGNSQLFT

SEQ ID NO: 915 - protein, 120 aa, Artificial Sequence; identical to SEQ ID NO: 539
  Recombinant protein

SEQ ID NO: 916 - protein, 5 aa, Artificial Sequence
  Recombinant protein
TSWIV

SEQ ID NO: 917 - protein, 17 aa, Artificial Sequence
  Recombinant protein
MIDPSDSYTSYSPSFQG

SEQ ID NO: 918 - protein, 11 aa, Artificial Sequence
  Recombinant protein
LHYYNSEEFDV

SEQ ID NO: 919 - protein, 109 aa, Artificial Sequence; identical to SEQ ID NO: 727
  Recombinant protein

SEQ ID NO: 920 - protein, 12 aa, Artificial Sequence
  Recombinant protein
RASQSVSSSYLA

SEQ ID NO: 921 - protein, 7 aa, Artificial Sequence
  Recombinant protein
GQSSRTR

SEQ ID NO: 922 - protein, 10 aa, Artificial Sequence
  Recombinant protein
QQFGNSQLFT

SEQ ID NO: 923 - protein, 120 aa, Artificial Sequence; identical to SEQ ID NO: 547
  Recombinant protein

SEQ ID NO: 924 - protein, 5 aa, Artificial Sequence
  Recombinant protein
TSWIV

SEQ ID NO: 925 - protein, 17 aa, Artificial Sequence
  Recombinant protein
MIDPSDSYTSYSPSFQG

SEQ ID NO: 926 - protein, 11 aa, Artificial Sequence
  Recombinant protein
LHYYNSEEFDV

SEQ ID NO: 927 - protein, 109 aa, Artificial Sequence; identical to SEQ ID NO: 727
  Recombinant protein

SEQ ID NO: 928 - protein, 12 aa, Artificial Sequence
  Recombinant protein
RASQSVSSSYLA

SEQ ID NO: 929 - protein, 7 aa, Artificial Sequence
  Recombinant protein
GQSSRTR

SEQ ID NO: 930 - protein, 10 aa, Artificial Sequence
  Recombinant protein
QQFGNSQLFT

SEQ ID NO: 931 - protein, 120 aa, Artificial Sequence; identical to SEQ ID NO: 483
  Recombinant protein

SEQ ID NO: 932 - protein, 5 aa, Artificial Sequence
  Recombinant protein
TSWIS

SEQ ID NO: 933 - protein, 17 aa, Artificial Sequence
  Recombinant protein
MIDPSDSYTSYSPSFQG

SEQ ID NO: 934 - protein, 11 aa, Artificial Sequence
  Recombinant protein
LHYYNSEEFDV

SEQ ID NO: 935 - protein, 109 aa, Artificial Sequence; identical to SEQ ID NO: 735
  Recombinant protein

SEQ ID NO: 936 - protein, 12 aa, Artificial Sequence
  Recombinant protein
RASQSVSSSYLA

SEQ ID NO: 937 - protein, 7 aa, Artificial Sequence
  Recombinant protein
GQSSRTR

SEQ ID NO: 938 - protein, 10 aa, Artificial Sequence
  Recombinant protein
QQFGQSQLFT

SEQ ID NO: 939 - protein, 120 aa, Artificial Sequence; identical to SEQ ID NO: 507
  Recombinant protein

SEQ ID NO: 940 - protein, 5 aa, Artificial Sequence
  Recombinant protein
TSWIS

SEQ ID NO: 941 - protein, 17 aa, Artificial Sequence
  Recombinant protein
MIDPSDSFTSYSPSFQG

SEQ ID NO: 942 - protein, 11 aa, Artificial Sequence
  Recombinant protein
LHYYHSEEFDV

SEQ ID NO: 943 - protein, 109 aa, Artificial Sequence; identical to SEQ ID NO: 735
  Recombinant protein

SEQ ID NO: 944 - protein, 12 aa, Artificial Sequence
  Recombinant protein
RASQSVSSSYLA

SEQ ID NO: 945 - protein, 7 aa, Artificial Sequence
  Recombinant protein
GQSSRTR

SEQ ID NO: 946 - protein, 10 aa, Artificial Sequence
  Recombinant protein
QQFGQSQLFT

SEQ ID NO: 947 - protein, 120 aa, Artificial Sequence; identical to SEQ ID NO: 523
  Recombinant protein

SEQ ID NO: 948 - protein, 5 aa, Artificial Sequence
  Recombinant protein
TSWIS

SEQ ID NO: 949 - protein, 17 aa, Artificial Sequence
  Recombinant protein
MIDPSDSYTSYSPSFQG

SEQ ID NO: 950 - protein, 11 aa, Artificial Sequence
  Recombinant protein
LHYYHSEEFDV

SEQ ID NO: 951 - protein, 109 aa, Artificial Sequence; identical to SEQ ID NO: 735
  Recombinant protein

SEQ ID NO: 952 - protein, 12 aa, Artificial Sequence
  Recombinant protein
RASQSVSSSYLA

SEQ ID NO: 953 - protein, 7 aa, Artificial Sequence
  Recombinant protein
GQSSRTR

SEQ ID NO: 954 - protein, 10 aa, Artificial Sequence
  Recombinant protein
QQFGQSQLFT

SEQ ID NO: 955 - protein, 120 aa, Artificial Sequence; identical to SEQ ID NO: 491
  Recombinant protein

SEQ ID NO: 956 - protein, 5 aa, Artificial Sequence
  Recombinant protein
TSWIS

SEQ ID NO: 957 - protein, 17 aa, Artificial Sequence
  Recombinant protein
MIDPSDSFTSYSPSFQG

SEQ ID NO: 958 - protein, 11 aa, Artificial Sequence
  Recombinant protein
LHYYNSEEFDV

SEQ ID NO: 959 - protein, 109 aa, Artificial Sequence; identical to SEQ ID NO: 735
  Recombinant protein

SEQ ID NO: 960 - protein, 12 aa, Artificial Sequence
  Recombinant protein
RASQSVSSSYLA

SEQ ID NO: 961 - protein, 7 aa, Artificial Sequence
  Recombinant protein
GQSSRTR

SEQ ID NO: 962 - protein, 10 aa, Artificial Sequence
  Recombinant protein
QQFGQSQLFT

SEQ ID NO: 963 - protein, 120 aa, Artificial Sequence; identical to SEQ ID NO: 539
  Recombinant protein

SEQ ID NO: 964 - protein, 5 aa, Artificial Sequence
  Recombinant protein
TSWIV

SEQ ID NO: 965 - protein, 17 aa, Artificial Sequence
  Recombinant protein
MIDPSDSYTSYSPSFQG

SEQ ID NO: 966 - protein, 11 aa, Artificial Sequence
  Recombinant protein
LHYYNSEEFDV

SEQ ID NO: 967 - protein, 109 aa, Artificial Sequence; identical to SEQ ID NO: 735
  Recombinant protein

SEQ ID NO: 968 - protein, 12 aa, Artificial Sequence
  Recombinant protein
RASQSVSSSYLA

SEQ ID NO: 969 - protein, 7 aa, Artificial Sequence
  Recombinant protein
GQSSRTR

SEQ ID NO: 970 - protein, 10 aa, Artificial Sequence
  Recombinant protein
QQFGQSQLFT

SEQ ID NO: 971 - protein, 120 aa, Artificial Sequence; identical to SEQ ID NO: 547
  Recombinant protein

SEQ ID NO: 972 - protein, 5 aa, Artificial Sequence
  Recombinant protein
TSWIV

SEQ ID NO: 973 - protein, 17 aa, Artificial Sequence
  Recombinant protein
MIDPSDSYTSYSPSFQG

SEQ ID NO: 974 - protein, 11 aa, Artificial Sequence
  Recombinant protein
LHYYNSEEFDV

SEQ ID NO: 975 - protein, 109 aa, Artificial Sequence; identical to SEQ ID NO: 735
  Recombinant protein

SEQ ID NO: 976 - protein, 12 aa, Artificial Sequence
  Recombinant protein
RASQSVSSSYLA

SEQ ID NO: 977 - protein, 7 aa, Artificial Sequence
  Recombinant protein
GQSSRTR

SEQ ID NO: 978 - protein, 10 aa, Artificial Sequence
  Recombinant protein
QQFGQSQLFT

SEQ ID NO: 979 - protein, 120 aa, Artificial Sequence; identical to SEQ ID NO: 483
  Recombinant protein

SEQ ID NO: 980 - protein, 5 aa, Artificial Sequence
  Recombinant protein
TSWIS

SEQ ID NO: 981 - protein, 17 aa, Artificial Sequence
  Recombinant protein
MIDPSDSYTSYSPSFQG

SEQ ID NO: 982 - protein, 11 aa, Artificial Sequence
  Recombinant protein
LHYYNSEEFDV

SEQ ID NO: 983 - protein, 109 aa, Artificial Sequence; identical to SEQ ID NO: 743
  Recombinant protein

SEQ ID NO: 984 - protein, 12 aa, Artificial Sequence
  Recombinant protein
RASQSVSSSYLA

SEQ ID NO: 985 - protein, 7 aa, Artificial Sequence
  Recombinant protein
GQSSRTR

SEQ ID NO: 986 - protein, 10 aa, Artificial Sequence
  Recombinant protein
QQFGDAQLFT

SEQ ID NO: 987 - protein, 120 aa, Artificial Sequence; identical to SEQ ID NO: 507
  Recombinant protein

SEQ ID NO: 988 - protein, 5 aa, Artificial Sequence
  Recombinant protein
TSWIS

SEQ ID NO: 989 - protein, 17 aa, Artificial Sequence
  Recombinant protein
MIDPSDSFTSYSPSFQG

SEQ ID NO: 990 - protein, 11 aa, Artificial Sequence
  Recombinant protein
LHYYHSEEFDV

SEQ ID NO: 991 - protein, 109 aa, Artificial Sequence; identical to SEQ ID NO: 743
  Recombinant protein

SEQ ID NO: 992 - protein, 12 aa, Artificial Sequence
  Recombinant protein
RASQSVSSSYLA

SEQ ID NO: 993 - protein, 7 aa, Artificial Sequence
  Recombinant protein
GQSSRTR

SEQ ID NO: 994 - protein, 10 aa, Artificial Sequence
  Recombinant protein
QQFGDAQLFT

SEQ ID NO: 995 - protein, 120 aa, Artificial Sequence; identical to SEQ ID NO: 523
  Recombinant protein

SEQ ID NO: 996 - protein, 5 aa, Artificial Sequence
  Recombinant protein
TSWIS

SEQ ID NO: 997 - protein, 17 aa, Artificial Sequence
  Recombinant protein
MIDPSDSYTSYSPSFQG

SEQ ID NO: 998 - protein, 11 aa, Artificial Sequence
  Recombinant protein
LHYYHSEEFDV

SEQ ID NO: 999 - protein, 109 aa, Artificial Sequence; identical to SEQ ID NO: 743
  Recombinant protein

SEQ ID NO: 1000 - protein, 12 aa, Artificial Sequence
  Recombinant protein
RASQSVSSSYLA

SEQ ID NO: 1001 - protein, 7 aa, Artificial Sequence
  Recombinant protein
GQSSRTR

SEQ ID NO: 1002 - protein, 10 aa, Artificial Sequence
  Recombinant protein
QQFGDAQLFT

SEQ ID NO: 1003 - protein, 120 aa, Artificial Sequence; identical to SEQ ID NO: 491
  Recombinant protein

SEQ ID NO: 1004 - protein, 5 aa, Artificial Sequence
  Recombinant protein
TSWIS

SEQ ID NO: 1005 - protein, 17 aa, Artificial Sequence
  Recombinant protein
MIDPSDSFTSYSPSFQG

SEQ ID NO: 1006 - protein, 11 aa, Artificial Sequence
  Recombinant protein
LHYYNSEEFDV

SEQ ID NO: 1007 - protein, 109 aa, Artificial Sequence; identical to SEQ ID NO: 743
  Recombinant protein

SEQ ID NO: 1008 - protein, 12 aa, Artificial Sequence
  Recombinant protein
RASQSVSSSYLA

SEQ ID NO: 1009 - protein, 7 aa, Artificial Sequence
  Recombinant protein
GQSSRTR

SEQ ID NO: 1010 - protein, 10 aa, Artificial Sequence
  Recombinant protein
QQFGDAQLFT

SEQ ID NO: 1011 - protein, 120 aa, Artificial Sequence; identical to SEQ ID NO: 539
  Recombinant protein

SEQ ID NO: 1012 - protein, 5 aa, Artificial Sequence
  Recombinant protein
TSWIV

SEQ ID NO: 1013 - protein, 17 aa, Artificial Sequence
  Recombinant protein
MIDPSDSYTSYSPSFQG

SEQ ID NO: 1014 - protein, 11 aa, Artificial Sequence
  Recombinant protein
LHYYNSEEFDV

SEQ ID NO: 1015 - protein, 109 aa, Artificial Sequence; identical to SEQ ID NO: 743
  Recombinant protein

SEQ ID NO: 1016 - protein, 12 aa, Artificial Sequence
  Recombinant protein
RASQSVSSSYLA

SEQ ID NO: 1017 - protein, 7 aa, Artificial Sequence
  Recombinant protein
GQSSRTR

SEQ ID NO: 1018 - protein, 10 aa, Artificial Sequence
  Recombinant protein
QQFGDAQLFT

SEQ ID NO: 1019 - protein, 120 aa, Artificial Sequence; identical to SEQ ID NO: 547
  Recombinant protein

SEQ ID NO: 1020 - protein, 5 aa, Artificial Sequence
  Recombinant protein
TSWIV

SEQ ID NO: 1021 - protein, 17 aa, Artificial Sequence
  Recombinant protein
MIDPSDSYTSYSPSFQG

SEQ ID NO: 1022 - protein, 11 aa, Artificial Sequence
  Recombinant protein
LHYYNSEEFDV

SEQ ID NO: 1023 - protein, 109 aa, Artificial Sequence; identical to SEQ ID NO: 743
  Recombinant protein

SEQ ID NO: 1024 - protein, 12 aa, Artificial Sequence
  Recombinant protein
RASQSVSSSYLA

SEQ ID NO: 1025 - protein, 7 aa, Artificial Sequence
  Recombinant protein
GQSSRTR

SEQ ID NO: 1026 - protein, 10 aa, Artificial Sequence
  Recombinant protein
QQFGDAQLFT

SEQ ID NO: 1027 - protein, 120 aa, Artificial Sequence; identical to SEQ ID NO: 483
  Recombinant protein

SEQ ID NO: 1028 - protein, 5 aa, Artificial Sequence
  Recombinant protein
TSWIS

SEQ ID NO: 1029 - protein, 17 aa, Artificial Sequence
  Recombinant protein
MIDPSDSYTSYSPSFQG

SEQ ID NO: 1030 - protein, 11 aa, Artificial Sequence
  Recombinant protein
LHYYNSEEFDV

SEQ ID NO: 1031 - protein, 109 aa, Artificial Sequence; identical to SEQ ID NO: 751
  Recombinant protein

SEQ ID NO: 1032 - protein, 12 aa, Artificial Sequence
  Recombinant protein
RASQSVSSSYLA

SEQ ID NO: 1033 - protein, 7 aa, Artificial Sequence
  Recombinant protein
GQSSRTR

SEQ ID NO: 1034 - protein, 10 aa, Artificial Sequence
  Recombinant protein
QQFGDTQLFT

SEQ ID NO: 1035 - protein, 120 aa, Artificial Sequence; identical to SEQ ID NO: 507
  Recombinant protein

SEQ ID NO: 1036 - protein, 5 aa, Artificial Sequence
  Recombinant protein
TSWIS

SEQ ID NO: 1037 - protein, 17 aa, Artificial Sequence
  Recombinant protein
MIDPSDSFTSYSPSFQG

SEQ ID NO: 1038 - protein, 11 aa, Artificial Sequence
  Recombinant protein
LHYYHSEEFDV

SEQ ID NO: 1039 - protein, 109 aa, Artificial Sequence; identical to SEQ ID NO: 751
  Recombinant protein

SEQ ID NO: 1040 - protein, 12 aa, Artificial Sequence
  Recombinant protein
RASQSVSSSYLA

SEQ ID NO: 1041 - protein, 7 aa, Artificial Sequence
  Recombinant protein
GQSSRTR

SEQ ID NO: 1042 - protein, 10 aa, Artificial Sequence
  Recombinant protein
QQFGDTQLFT

SEQ ID NO: 1043 - protein, 120 aa, Artificial Sequence; identical to SEQ ID NO: 515
  Recombinant protein

SEQ ID NO: 1044 - protein, 5 aa, Artificial Sequence
  Recombinant protein
TSWIS

SEQ ID NO: 1045 - protein, 17 aa, Artificial Sequence
  Recombinant protein
MIDPSDSYTSYSPSFQG

SEQ ID NO: 1046 - protein, 11 aa, Artificial Sequence
  Recombinant protein
LHYYHSEEFDV

SEQ ID NO: 1047 - protein, 109 aa, Artificial Sequence; identical to SEQ ID NO: 751
  Recombinant protein

SEQ ID NO: 1048 - protein, 12 aa, Artificial Sequence
  Recombinant protein
RASQSVSSSYLA

SEQ ID NO: 1049 - protein, 7 aa, Artificial Sequence
  Recombinant protein
GQSSRTR

SEQ ID NO: 1050 - protein, 10 aa, Artificial Sequence
  Recombinant protein
QQFGDTQLFT

SEQ ID NO: 1051 - protein, 120 aa, Artificial Sequence; identical to SEQ ID NO: 523
  Recombinant protein

SEQ ID NO: 1052 - protein, 5 aa, Artificial Sequence
  Recombinant protein
TSWIS

SEQ ID NO: 1053 - protein, 17 aa, Artificial Sequence
  Recombinant protein
MIDPSDSYTSYSPSFQG

SEQ ID NO: 1054 - protein, 11 aa, Artificial Sequence
  Recombinant protein
LHYYHSEEFDV

SEQ ID NO: 1055 - protein, 109 aa, Artificial Sequence; identical to SEQ ID NO: 751
  Recombinant protein

SEQ ID NO: 1056 - protein, 12 aa, Artificial Sequence
  Recombinant protein
RASQSVSSSYLA

SEQ ID NO: 1057 - protein, 7 aa, Artificial Sequence
  Recombinant protein
GQSSRTR

SEQ ID NO: 1058 - protein, 10 aa, Artificial Sequence
  Recombinant protein
QQFGDTQLFT

SEQ ID NO: 1059 - protein, 120 aa, Artificial Sequence; identical to SEQ ID NO: 491
  Recombinant protein

SEQ ID NO: 1060 - protein, 5 aa, Artificial Sequence
  Recombinant protein
TSWIS

SEQ ID NO: 1061 - protein, 17 aa, Artificial Sequence
  Recombinant protein
MIDPSDSFTSYSPSFQG

SEQ ID NO: 1062 - protein, 11 aa, Artificial Sequence
  Recombinant protein
LHYYNSEEFDV

SEQ ID NO: 1063 - protein, 109 aa, Artificial Sequence; identical to SEQ ID NO: 751
  Recombinant protein

SEQ ID NO: 1064 - protein, 12 aa, Artificial Sequence
  Recombinant protein
RASQSVSSSYLA

SEQ ID NO: 1065 - protein, 7 aa, Artificial Sequence
  Recombinant protein
GQSSRTR

SEQ ID NO: 1066 - protein, 10 aa, Artificial Sequence
  Recombinant protein
QQFGDTQLFT

SEQ ID NO: 1067 - protein, 120 aa, Artificial Sequence; identical to SEQ ID NO: 539
  Recombinant protein

SEQ ID NO: 1068 - protein, 5 aa, Artificial Sequence
  Recombinant protein
TSWIV

SEQ ID NO: 1069 - protein, 17 aa, Artificial Sequence
  Recombinant protein
MIDPSDSYTSYSPSFQG

SEQ ID NO: 1070 - protein, 11 aa, Artificial Sequence
  Recombinant protein
LHYYNSEEFDV

SEQ ID NO: 1071 - protein, 109 aa, Artificial Sequence; identical to SEQ ID NO: 751
  Recombinant protein

SEQ ID NO: 1072 - protein, 12 aa, Artificial Sequence
  Recombinant protein
RASQSVSSSYLA

SEQ ID NO: 1073 - protein, 7 aa, Artificial Sequence
  Recombinant protein
GQSSRTR

SEQ ID NO: 1074 - protein, 10 aa, Artificial Sequence
  Recombinant protein
QQFGDTQLFT

SEQ ID NO: 1075 - protein, 120 aa, Artificial Sequence; identical to SEQ ID NO: 547
  Recombinant protein

SEQ ID NO: 1076 - protein, 5 aa, Artificial Sequence
  Recombinant protein
TSWIV

SEQ ID NO: 1077 - protein, 17 aa, Artificial Sequence
  Recombinant protein
MIDPSDSYTSYSPSFQG

SEQ ID NO: 1078 - protein, 11 aa, Artificial Sequence
  Recombinant protein
LHYYNSEEFDV

SEQ ID NO: 1079 - protein, 109 aa, Artificial Sequence; identical to SEQ ID NO: 751
  Recombinant protein

SEQ ID NO: 1080 - protein, 12 aa, Artificial Sequence
  Recombinant protein
RASQSVSSSYLA

SEQ ID NO: 1081 - protein, 7 aa, Artificial Sequence
  Recombinant protein
GQSSRTR

SEQ ID NO: 1082 - protein, 10 aa, Artificial Sequence
  Recombinant protein
QQFGDTQLFT

SEQ ID NO: 1083 - protein, 120 aa, Artificial Sequence; identical to SEQ ID NO: 483
  Recombinant protein

SEQ ID NO: 1084 - protein, 5 aa, Artificial Sequence
  Recombinant protein
TSWIS

SEQ ID NO: 1085 - protein, 17 aa, Artificial Sequence
  Recombinant protein
MIDPSDSYTSYSPSFQG

SEQ ID NO: 1086 - protein, 11 aa, Artificial Sequence
  Recombinant protein
LHYYNSEEFDV

SEQ ID NO: 1087 - protein, 109 aa, Artificial Sequence; identical to SEQ ID NO: 759
  Recombinant protein

SEQ ID NO: 1088 - protein, 12 aa, Artificial Sequence
  Recombinant protein
RASQSVSSSYLA

SEQ ID NO: 1089 - protein, 7 aa, Artificial Sequence
  Recombinant protein
GQSSRTR

SEQ ID NO: 1090 - protein, 10 aa, Artificial Sequence
  Recombinant protein
QQFGDNQLFT

SEQ ID NO: 1091 - protein, 120 aa, Artificial Sequence; identical to SEQ ID NO: 507
  Recombinant protein

SEQ ID NO: 1092 - protein, 5 aa, Artificial Sequence
  Recombinant protein
TSWIS

SEQ ID NO: 1093 - protein, 17 aa, Artificial Sequence
  Recombinant protein
MIDPSDSFTSYSPSFQG

SEQ ID NO: 1094 - protein, 11 aa, Artificial Sequence
  Recombinant protein
LHYYHSEEFDV

SEQ ID NO: 1095 - protein, 109 aa, Artificial Sequence; identical to SEQ ID NO: 759
  Recombinant protein

SEQ ID NO: 1096 - protein, 12 aa, Artificial Sequence
  Recombinant protein
RASQSVSSSYLA

SEQ ID NO: 1097 - protein, 7 aa, Artificial Sequence
  Recombinant protein
GQSSRTR

SEQ ID NO: 1098 - protein, 10 aa, Artificial Sequence
  Recombinant protein
QQFGDNQLFT

SEQ ID NO: 1099 - protein, 120 aa, Artificial Sequence; identical to SEQ ID NO: 515
  Recombinant protein

SEQ ID NO: 1100 - protein, 5 aa, Artificial Sequence
  Recombinant protein
TSWIS

SEQ ID NO: 1101 - protein, 17 aa, Artificial Sequence
  Recombinant protein
MIDPSDSYTSYSPSFQG

SEQ ID NO: 1102 - protein, 11 aa, Artificial Sequence
  Recombinant protein
LHYYHSEEFDV

SEQ ID NO: 1103 - protein, 109 aa, Artificial Sequence; identical to SEQ ID NO: 759
  Recombinant protein

SEQ ID NO: 1104 - protein, 12 aa, Artificial Sequence
  Recombinant protein
RASQSVSSSYLA

SEQ ID NO: 1105 - protein, 7 aa, Artificial Sequence
  Recombinant protein
GQSSRTR

SEQ ID NO: 1106 - protein, 10 aa, Artificial Sequence
  Recombinant protein
QQFGDNQLFT

SEQ ID NO: 1107 - protein, 120 aa, Artificial Sequence; identical to SEQ ID NO: 523
  Recombinant protein

SEQ ID NO: 1108 - protein, 5 aa, Artificial Sequence
  Recombinant protein
TSWIS

SEQ ID NO: 1109 - protein, 17 aa, Artificial Sequence
  Recombinant protein
MIDPSDSYTSYSPSFQG

SEQ ID NO: 1110 - protein, 11 aa, Artificial Sequence
  Recombinant protein
LHYYHSEEFDV

SEQ ID NO: 1111 - protein, 109 aa, Artificial Sequence; identical to SEQ ID NO: 759
  Recombinant protein

SEQ ID NO: 1112 - protein, 12 aa, Artificial Sequence
  Recombinant protein
RASQSVSSSYLA

SEQ ID NO: 1113 - protein, 7 aa, Artificial Sequence
  Recombinant protein
GQSSRTR

SEQ ID NO: 1114 - protein, 10 aa, Artificial Sequence
  Recombinant protein
QQFGDNQLFT

SEQ ID NO: 1115 - protein, 120 aa, Artificial Sequence; identical to SEQ ID NO: 491
  Recombinant protein

SEQ ID NO: 1116 - protein, 5 aa, Artificial Sequence
  Recombinant protein
TSWIS

SEQ ID NO: 1117 - protein, 17 aa, Artificial Sequence
  Recombinant protein
MIDPSDSFTSYSPSFQG

SEQ ID NO: 1118 - protein, 11 aa, Artificial Sequence
  Recombinant protein
LHYYNSEEFDV

SEQ ID NO: 1119 - protein, 109 aa, Artificial Sequence; identical to SEQ ID NO: 759
  Recombinant protein

SEQ ID NO: 1120 - protein, 12 aa, Artificial Sequence
  Recombinant protein
RASQSVSSSYLA

SEQ ID NO: 1121 - protein, 7 aa, Artificial Sequence
  Recombinant protein
GQSSRTR

SEQ ID NO: 1122 - protein, 10 aa, Artificial Sequence
  Recombinant protein
QQFGDNQLFT

SEQ ID NO: 1123 - protein, 120 aa, Artificial Sequence; identical to SEQ ID NO: 539
  Recombinant protein

SEQ ID NO: 1124 - protein, 5 aa, Artificial Sequence
  Recombinant protein
TSWIV

SEQ ID NO: 1125 - protein, 17 aa, Artificial Sequence
  Recombinant protein
MIDPSDSYTSYSPSFQG

SEQ ID NO: 1126 - protein, 11 aa, Artificial Sequence
  Recombinant protein
LHYYNSEEFDV

SEQ ID NO: 1127 - protein, 109 aa, Artificial Sequence; identical to SEQ ID NO: 759
  Recombinant protein

SEQ ID NO: 1128 - protein, 12 aa, Artificial Sequence
  Recombinant protein
RASQSVSSSYLA

SEQ ID NO: 1129 - protein, 7 aa, Artificial Sequence
  Recombinant protein
GQSSRTR

SEQ ID NO: 1130 - protein, 10 aa, Artificial Sequence
  Recombinant protein
QQFGDNQLFT

SEQ ID NO: 1131 - protein, 120 aa, Artificial Sequence; identical to SEQ ID NO: 547
  Recombinant protein

SEQ ID NO: 1132 - protein, 5 aa, Artificial Sequence
  Recombinant protein
TSWIV

SEQ ID NO: 1133 - protein, 17 aa, Artificial Sequence
  Recombinant protein
MIDPSDSYTSYSPSFQG

SEQ ID NO: 1134 - protein, 11 aa, Artificial Sequence
  Recombinant protein
LHYYNSEEFDV

SEQ ID NO: 1135 - protein, 109 aa, Artificial Sequence; identical to SEQ ID NO: 759
  Recombinant protein

SEQ ID NO: 1136 - protein, 12 aa, Artificial Sequence
  Recombinant protein
RASQSVSSSYLA

SEQ ID NO: 1137 - protein, 7 aa, Artificial Sequence
  Recombinant protein
GQSSRTR

SEQ ID NO: 1138 - protein, 10 aa, Artificial Sequence
  Recombinant protein
QQFGDNQLFT

SEQ ID NO: 1139 - protein, 120 aa, Artificial Sequence; identical to SEQ ID NO: 483
  Recombinant protein

SEQ ID NO: 1140 - protein, 5 aa, Artificial Sequence
  Recombinant protein
TSWIS

SEQ ID NO: 1141 - protein, 17 aa, Artificial Sequence
  Recombinant protein
MIDPSDSYTSYSPSFQG

SEQ ID NO: 1142 - protein, 11 aa, Artificial Sequence
  Recombinant protein
LHYYNSEEFDV

SEQ ID NO: 1143 - protein, 109 aa, Artificial Sequence
  Recombinant protein
EIVLTQSPGTLSLSPGERATLSCRASQSVSSSYLAWYQQKPGQAPRLLIYGQSSRTRGIPDRFSGSGSGTDFTLTISRLEPEDFAVYYCQQFGDDQLFTFGQGTKLEIK

SEQ ID NO: 1144 - protein, 12 aa, Artificial Sequence
  Recombinant protein
RASQSVSSSYLA

SEQ ID NO: 1145 - protein, 7 aa, Artificial Sequence
  Recombinant protein
GQSSRTR

SEQ ID NO: 1146 - protein, 10 aa, Artificial Sequence
  Recombinant protein
QQFGDDQLFT

SEQ ID NO: 1147 - protein, 120 aa, Artificial Sequence; identical to SEQ ID NO: 507
  Recombinant protein

SEQ ID NO: 1148 - protein, 5 aa, Artificial Sequence
  Recombinant protein
TSWIS

SEQ ID NO: 1149 - protein, 17 aa, Artificial Sequence
  Recombinant protein
MIDPSDSFTSYSPSFQG

SEQ ID NO: 1150 - protein, 11 aa, Artificial Sequence
  Recombinant protein
LHYYHSEEFDV

SEQ ID NO: 1151 - protein, 109 aa, Artificial Sequence; identical to SEQ ID NO: 1143
  Recombinant protein

SEQ ID NO: 1152 - protein, 12 aa, Artificial Sequence
  Recombinant protein
RASQSVSSSYLA

SEQ ID NO: 1153 - protein, 7 aa, Artificial Sequence
  Recombinant protein
GQSSRTR

SEQ ID NO: 1154 - protein, 10 aa, Artificial Sequence
  Recombinant protein
QQFGDDQLFT

SEQ ID NO: 1155 - protein, 120 aa, Artificial Sequence; identical to SEQ ID NO: 515
  Recombinant protein

SEQ ID NO: 1156 - protein, 5 aa, Artificial Sequence
  Recombinant protein
TSWIS

SEQ ID NO: 1157 - protein, 17 aa, Artificial Sequence
  Recombinant protein
MIDPSDSYTSYSPSFQG

SEQ ID NO: 1158 - protein, 11 aa, Artificial Sequence
  Recombinant protein
LHYYHSEEFDV

SEQ ID NO: 1159 - protein, 109 aa, Artificial Sequence; identical to SEQ ID NO: 1143
  Recombinant protein

SEQ ID NO: 1160 - protein, 12 aa, Artificial Sequence
  Recombinant protein
RASQSVSSSYLA

SEQ ID NO: 1161 - protein, 7 aa, Artificial Sequence
  Recombinant protein
GQSSRTR

SEQ ID NO: 1162 - protein, 10 aa, Artificial Sequence
  Recombinant protein
QQFGDDQLFT

SEQ ID NO: 1163 - protein, 120 aa, Artificial Sequence; identical to SEQ ID NO: 523
  Recombinant protein

SEQ ID NO: 1164 - protein, 5 aa, Artificial Sequence
  Recombinant protein
TSWIS

SEQ ID NO: 1165 - protein, 17 aa, Artificial Sequence
  Recombinant protein
MIDPSDSYTSYSPSFQG

SEQ ID NO: 1166 - protein, 11 aa, Artificial Sequence
  Recombinant protein
LHYYHSEEFDV

SEQ ID NO: 1167 - protein, 109 aa, Artificial Sequence; identical to SEQ ID NO: 1143
  Recombinant protein

SEQ ID NO: 1168 - protein, 12 aa, Artificial Sequence
  Recombinant protein
RASQSVSSSYLA

SEQ ID NO: 1169 - protein, 7 aa, Artificial Sequence
  Recombinant protein
GQSSRTR

SEQ ID NO: 1170 - protein, 10 aa, Artificial Sequence
  Recombinant protein
QQFGDDQLFT

SEQ ID NO: 1171 - protein, 120 aa, Artificial Sequence; identical to SEQ ID NO: 491
  Recombinant protein

SEQ ID NO: 1172 - protein, 5 aa, Artificial Sequence
  Recombinant protein
TSWIS

SEQ ID NO: 1173 - protein, 17 aa, Artificial Sequence
  Recombinant protein
MIDPSDSFTSYSPSFQG

SEQ ID NO: 1174 - protein, 11 aa, Artificial Sequence
  Recombinant protein
LHYYNSEEFDV

SEQ ID NO: 1175 - protein, 109 aa, Artificial Sequence; identical to SEQ ID NO: 1143
  Recombinant protein

SEQ ID NO: 1176 - protein, 12 aa, Artificial Sequence
  Recombinant protein
RASQSVSSSYLA

SEQ ID NO: 1177 - protein, 7 aa, Artificial Sequence
  Recombinant protein
GQSSRTR

SEQ ID NO: 1178 - protein, 10 aa, Artificial Sequence
  Recombinant protein
QQFGDDQLFT

SEQ ID NO: 1179 - protein, 120 aa, Artificial Sequence; identical to SEQ ID NO: 539
  Recombinant protein

SEQ ID NO: 1180 - protein, 5 aa, Artificial Sequence
  Recombinant protein
TSWIV

SEQ ID NO: 1181 - protein, 17 aa, Artificial Sequence
  Recombinant protein
MIDPSDSYTSYSPSFQG

SEQ ID NO: 1182 - protein, 11 aa, Artificial Sequence
  Recombinant protein
LHYYNSEEFDV

SEQ ID NO: 1183 - protein, 109 aa, Artificial Sequence; identical to SEQ ID NO: 1143
  Recombinant protein

SEQ ID NO: 1184 - protein, 12 aa, Artificial Sequence
  Recombinant protein
RASQSVSSSYLA

SEQ ID NO: 1185 - protein, 7 aa, Artificial Sequence
  Recombinant protein
GQSSRTR

SEQ ID NO: 1186 - protein, 10 aa, Artificial Sequence
  Recombinant protein
QQFGDDQLFT

SEQ ID NO: 1187 - protein, 120 aa, Artificial Sequence; identical to SEQ ID NO: 547
  Recombinant protein

SEQ ID NO: 1188 - protein, 5 aa, Artificial Sequence
  Recombinant protein
TSWIV

SEQ ID NO: 1189 - protein, 17 aa, Artificial Sequence
  Recombinant protein
MIDPSDSYTSYSPSFQG

SEQ ID NO: 1190 - protein, 11 aa, Artificial Sequence
  Recombinant protein
LHYYNSEEFDV

SEQ ID NO: 1191 - protein, 109 aa, Artificial Sequence; identical to SEQ ID NO: 1143
  Recombinant protein

SEQ ID NO: 1192 - protein, 12 aa, Artificial Sequence
  Recombinant protein
RASQSVSSSYLA

SEQ ID NO: 1193 - protein, 7 aa, Artificial Sequence
  Recombinant protein
GQSSRTR

SEQ ID NO: 1194 - protein, 10 aa, Artificial Sequence
  Recombinant protein
QQFGDDQLFT

SEQ ID NO: 1195 - protein, 326 aa, Artificial Sequence
  Recombinant protein
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 1196 - protein, 326 aa, Artificial Sequence
  Recombinant protein
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFLLYSKLTVDKSRWQEGNVFSCSVMHEALHNHYTQKSLSLSLG

SEQ ID NO: 1197 - protein, 107 aa, Homo sapiens [sequence omitted]

SEQ ID NO: 1198 - protein, 329 aa, Artificial Sequence
  Recombinant protein
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVMHEALHNHYTQKSLSLSPG

SEQ ID NO: 1199 - protein, 329 aa, Artificial Sequence
  Recombinant protein
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSRLTVDKSRWQQGNVFSCSVMHEALHNHYTQKSLSLSPG

SEQ ID NO: 1200 - protein, 6 aa, Artificial Sequence
  Recombinant peptide
HHHHHH

SEQ ID NO: 1201 - protein, 10 aa, Artificial Sequence
  Recombinant peptide
GGGGSGGGGS

SEQ ID NO: 1202 - protein, 122 aa, Artificial Sequence
  Recombinant Protein
EVQLVESGGGLVQPGRSLRLSCAASGFTFHDYAMHWVRQAPGKGLEWVSGISWKGDIGGYVDSVKGRFTISRDNAKNSLYLQMNSLRAEDTALYYCAKSYGSGSFYNAFDSWGQGTLVTVSS

SEQ ID NO: 1203 - protein, 5 aa, Artificial Sequence
  Recombinant Protein
DYAMH

SEQ ID NO: 1204 - protein, 17 aa, Artificial Sequence
  Recombinant Protein
GISWKGDIGGYVDSVKG

SEQ ID NO: 1205 - protein, 13 aa, Artificial Sequence
  Recombinant Protein
SYGSGSFYNAFDS

SEQ ID NO: 1206 - protein, 107 aa, Homo sapiens; identical to SEQ ID NO: 127

SEQ ID NO: 1207 - protein, 11 aa, Homo sapiens
RASQSISSWLA

SEQ ID NO: 1208 - protein, 7 aa, Artificial Sequence
  Recombinant Protein
KASKLDR

SEQ ID NO: 1209 - protein, 9 aa, Artificial Sequence
  Recombinant Sequence
LEYSSYIRT

SEQ ID NO: 1210 - protein, 122 aa, Artificial Sequence; identical to SEQ ID NO: 51
  Recombinant Protein

SEQ ID NO: 1211 - protein, 5 aa, Artificial Sequence
  Recombinant Protein
DYAMH

SEQ ID NO: 1212 - protein, 17 aa, Artificial Sequence
  Recombinant Protein
GISWRGDIGGYVKSVKG

SEQ ID NO: 1213 - protein, 13 aa, Artificial Sequence
  Recombinant Protein
SYGSGSFYNAFDS

SEQ ID NO: 1214 - protein, 107 aa, Artificial Sequence
  Recombinant Protein
DIQMTQSPSTLSASVGDEVTITCRASQSISSWLAWYQQKPGKAPKFLIYKASKLERGTPSRFSGSGSGTEFSLTISSLQPDDFATYYCLEYSSYIRTFGQGTKVEIK

SEQ ID NO: 1215 - protein, 11 aa, Artificial Sequence
  Recombinant Protein
RASQSISSWLA

SEQ ID NO: 1216 - protein, 7 aa, Artificial Sequence
  Recombinant Protein
KASKLER

SEQ ID NO: 1217 - protein, 9 aa, Artificial Sequence
  Recombinant Protein
LEYSSYIRT

SEQ ID NO: 1218 - protein, 122 aa, Artificial Sequence
  Recombinant Protein
EVQLVESGGGLVQPGRSLRLSCAASGFTFHDYAMHWVRQVPGKGLEWVSGISWRGDIGGYAKSVKGRFTISRDNAKNSLYLQMNSLRAEDTALYYCVKSYGSGSFYNAFDSWGQGTLVTVSS

SEQ ID NO: 1219 - protein, 5 aa, Artificial Sequence
  Recombinant Protein
DYAMH

SEQ ID NO: 1220 - protein, 17 aa, Artificial Sequence
  Recombinant Protein
GISWRGDIGGYAKSVKG

SEQ ID NO: 1221 - protein, 13 aa, Artificial Sequence
  Recombinant Protein
SYGSGSFYNAFDS

SEQ ID NO: 1222 - protein, 107 aa, Artificial Sequence; identical to SEQ ID NO: 127
  Recombinant Protein

SEQ ID NO: 1223 - protein, 11 aa, Homo sapiens
RASQSISSWLA

SEQ ID NO: 1224 - protein, 7 aa, Homo sapiens
KASKLDR

SEQ ID NO: 1225 - protein, 9 aa, Artificial Sequence
  Recombinant Protein
LEYSSYIRT

SEQ ID NO: 1226 - protein, 122 aa, Artificial Sequence
  Recombinant Protein
EVQLVESGGGLVQPGRSLRLSCAASGFTFDDYAMHWVRQVPGKGLEWVSGISWRGDIGGYVKSVKGRFTISRDNAKNSLYLQMNSLRAEDTALYYCVKSYGSGSFYNAFDSWGQGTLVTVSS

SEQ ID NO: 1227 - protein, 5 aa, Artificial Sequence
  Recombinant Protein
DYAMH

SEQ ID NO: 1228 - protein, 17 aa, Artificial Sequence
  Recombinant Protein
GISWRGDIGGYVKSVKG

SEQ ID NO: 1229 - protein, 13 aa, Artificial Sequence
  Recombinant Protein
SYGSGSFYNAFDS

SEQ ID NO: 1230 - protein, 107 aa, Artificial Sequence; identical to SEQ ID NO: 127
  Recombinant Protein

SEQ ID NO: 1231 - protein, 11 aa, Artificial Sequence
  Recombinant Protein
RASQSISSWLA

SEQ ID NO: 1232 - protein, 7 aa, Artificial Sequence
  Recombinant Protein
KASKLDR

SEQ ID NO: 1233 - protein, 9 aa, Artificial Sequence
  Recombinant Protein
LEYSSYIRT

SEQ ID NO: 1234 - protein, 122 aa, Artificial Sequence; identical to SEQ ID NO: 1226
  Recombinant Protein

SEQ ID NO: 1235 - protein, 5 aa, Artificial Sequence
  Recombinant Protein
DYAMH

SEQ ID NO: 1236 - protein, 17 aa, Artificial Sequence
  Recombinant Protein
GISWRGDIGGYVKSVKG

SEQ ID NO: 1237 - protein, 13 aa, Artificial Sequence
  Recombinant Protein
SYGSGSFYNAFDS

SEQ ID NO: 1238 - protein, 107 aa, Artificial Sequence
  Recombinant Protein
DIQMTQSPSTLSASVGDRVTITCRASQSISSWLAWYQQKPGKAPKLLIYKASKLDRGTPSRFSGSGSGTEFTLTISSLQPDDFATYYCLEYSSYIRTFGQGTKVEIK

SEQ ID NO: 1239 - protein, 11 aa, Artificial Sequence
  Recombinant Protein
RASQSISSWLA

SEQ ID NO: 1240 - protein, 7 aa, Artificial Sequence
  Recombinant Protein
KASKLDR

SEQ ID NO: 1241 - protein, 9 aa, Artificial Sequence
  Recombinant Protein
LEYSSYIRT

SEQ ID NO: 1242 - protein, 122 aa, Artificial Sequence; identical to SEQ ID NO: 51
  Recombinant Protein

SEQ ID NO: 1243 - protein, 5 aa, Artificial Sequence
  Recombinant Protein
DYAMH

SEQ ID NO: 1244 - protein, 17 aa, Artificial Sequence
  Recombinant Protein
GISWRGDIGGYVKSVKG

SEQ ID NO: 1245 - protein, 13 aa, Artificial Sequence
  Recombinant Protein
SYGSGSFYNAFDS

SEQ ID NO: 1246 - protein, 107 aa, Artificial Sequence; identical to SEQ ID NO: 127
  Recombinant Protein

SEQ ID NO: 1247 - protein, 11 aa, Artificial Sequence
  Recombinant Protein
RASQSISSWLA

SEQ ID NO: 1248 - protein, 7 aa, Artificial Sequence
  Recombinant Protein
KASKLDR

SEQ ID NO: 1249 - protein, 9 aa, Artificial Sequence
  Recombinant Protein
LEYSSYIRT

(19) United States
(12) Patent Application Publication
(10) Pub. No.: US 2023/0058721 A1
(43) Pub. Date: Feb. 23, 2023

(54) Procoagulant Antibodies

(71) Applicant: Novo Nordisk A/S, Bagsvaerd (DK)

(72) Inventors: Karina Thorn, Farum (DK); Bjarne Gram Hansen, Alleroed (DK); Laust Bruun Johnsen, Skodsborg (DK); Mikkel Nors Harndahl, Roskilde (DK); Zhiru Yang, Beijing (CN); Henrik Oestergaard, Oelstykke (DK); Per J. Greisen, Belmont, MA (US); Eva Johansson, Bagsvaerd (DK); Morten Groenbech Rasch, Smoerum (DK); Jianhe Chen, Beijing (CN); Anders Svensson, Malmoe (SE); Haisun Zhu, Norfolk, MA (US); Rong Zhou, Beijing (CN); Prafull S. Gandhi, Ballerup (DK)

(21) Appl. No.: 17/522,949

(22) Filed: Nov. 10, 2021

Related U.S. Application Data
(63) Continuation of application No. 17/161,741, filed on Jan. 29, 2021, now Pat. No. 11,220,554.
Application No. 17/161,741 is a continuation of application No. PCT/EP2019/070628, filed on Jul. 31, 2019.

(30) Foreign Application Priority Data
Aug. 1, 2018 (CN) PCT/CN2018/097834
Aug. 8, 2018 (CN) PCT/CN2018/099339
Sep. 7, 2018 (EP) 18193191.6

(51) Int. Cl. C07K 16/36; A61P 7/04

(52) U.S. Cl. CPC C07K 16/36; A61K 2039/505; A61P 7/04

(57) ABSTRACT

The present invention relates to multispecific procoagulant antibodies capable of binding to coagulation Factor IX (FIX) and/or the activated form thereof Factor IXa (FIXa), and Factor X (FX) and/or the activated form thereof Factor Xa (FXa) and promoting FX activation by FIXa, antibodies binding their epitopes or parts thereof and methods and composition for treating subjects suffering from a coagulopathy such as haemophilia A as well as kits, methods of manufacture and methods of use.

### CROSS-REFERENCE TO RELATED APPLICATIONS

This application is a continuation of U.S. application Ser. No. 17/161,741, filed Jan. 29, 2021, which is a continuation of an International Application No. PCT/EP2019/070628 (WO 2020/025672), filed Jul. 31, 2019, which claims priority to European Patent Application No. 18193191.6, filed Sep. 7, 2018, International Application No. PCT/CN2018/099339, filed Aug. 8, 2018, and International Application No. PCT/CN2018/097834, filed Aug. 1, 2018; the contents all of which are incorporated herein by reference.

### SEQUENCE LISTING

The instant application contains a Sequence Listing which has been submitted in ASCII format via EFS-Web and is hereby incorporated by reference in its entirety. Said ASCII copy, created on Oct. 8, 2021, is named 180021US02_SeqList.txt and is 541 kilobytes in size.

### BACKGROUND

In patients with a coagulopathy, such as in human beings with haemophilia A and B, various steps of the coagulation cascade are rendered dysfunctional due to, for example, the absence or insufficient presence of a functional coagulation factor. Such dysfunction of one part of the coagulation cascade results in insufficient blood coagulation and potentially life-threatening bleeding, or damage to internal organs, such as the joints.

Coagulation Factor VIII (FVIII) deficiency, commonly referred to as haemophilia A, is a congenital bleeding disorder affecting approximately 420,000 people worldwide, of which around 105,000 are currently diagnosed.

Patients with haemophilia A may receive coagulation factor replacement therapy such as exogenous FVIII. Conventional treatment consists of replacement therapy, provided as prophylaxis or on demand treatment of bleeding episodes. Until recently prophylactic treatment for a patient with severe haemophilia A was up to three intravenous injections/week with either plasma derived FVIII or recombinant FVIII or long-acting variants thereof.

However, such patients are at risk of developing neutralizing antibodies, so-called inhibitors, to such exogenous factors, rendering formerly efficient therapy ineffective. Haemophilia A patients with inhibitors is a non-limiting example of a coagulopathy that is partly congenital and partly acquired. Patients that have developed inhibitors to FVIII cannot be treated with conventional replacement therapy. Exogenous coagulation factors may only be administered intravenously, which is of considerable inconvenience and discomfort to patients. For example, infants and toddlers may have to have intravenous catheters surgically inserted into a chest vein, in order for venous access to be guaranteed. This leaves them at great risk of developing bacterial infections.

In a bleeding individual, coagulation is initiated by formation of the Tissue Factor/Factor Vila (TF/FVIIa) complex when extravascular TF is exposed to activated FVII (FVIIa) in the blood. TF/FVIIa complex formation leads to the activation of coagulation Factor X (FX) to activated coagulation Factor Xa (FXa) which, together with activated coagulation Factor V (FVa), generates a limited amount of thrombin, which in turn activates blood platelets. Activated platelets support the assembly of the tenase complex composed of activated Factor VIII (FVIIIa) and activated coagulation Factor IX (FIXa). The tenase complex is a very efficient catalyst of FX activation and FXa generated in this second step serves as the active protease in the FVa/FXa pro-thrombinase complex which is responsible for the final thrombin burst. Thrombin cleaves fibrinogen to generate fibrin monomers, which polymerise to form a fibrin network which seals the leaking vessel and stops the bleeding. The rapid and extensive thrombin burst is a prerequisite for the formation of a solid and stable fibrin clot.

An inadequate FXa formation and decreased thrombin generation caused by reduced or absent FVIII activity is the reason underlying the bleeding diathesis in haemophilia A patients.

As mentioned, proteolytic conversion of FX into its enzymatically active form FXa can be achieved by the intrinsic FX-activating complex comprising FIXa and its cofactor FVIIIa. Cofactor binding increases the enzymatic activity of FIXa by about five orders of magnitude and is believed to result through multiple mechanisms as outlined by Scheiflinger et al. (2008) J Thromb Haemost, 6:315-322. Notably, FVIIIa has been found to stabilize a conformation of FIXa that has increased proteolytic activity towards FX (Kolkman J A, Mertens K (2000) Biochemistry, 39:7398-7405, Zögg T, Brandstetter H (2009) Biol Chem, 390:391-400). Based on this observation and realizing that antibodies are versatile binding proteins capable of mimicking a variety of protein-protein interactions, Scheiflinger et al. performed a screen for agonistic anti-FIXa antibodies characterized by an ability to enhance FX activation by FIXa in the presence of a phospholipid surface and calcium, but in the absence of the natural cofactor FVIIIa. From a screen of 5280 hybridoma supernatants, 88 were found to produce antibodies exhibiting various degrees of FIXa agonistic activity, cf. EP1220923 B1 and EP1660536 B1. With respect to the kinetics of FX activation and ability to stimulate thrombin generation in FVIII-deficient human plasma, EP1660536 B1 consistently points to the anti-FIXa antibody 224F3 as the most efficient antibody.

Recently, a new drug, emicizumab (HEMLIBRA®) also known as ACE910, has been approved for subcutaneous prophylactic treatment of Haemophilia A with or without inhibitors against conventional replacement therapy factors. Emicizumab is a humanized, bispecific anti-FIX(a)/anti-FX(a) monoclonal antibody developed by Chugai Pharmaceuticals/Roche Pharmaceuticals for the treatment of haemophilia A. Emicizumab is designed to mimic FVIII cofactor function (see Sampei et al.: (2013) PLoS One, 8, e57479 and WO2012067176), however, some patients have developed inhibitors against emicizumab rendering treatment with this compound ineffective.

There are still many unmet medical needs in the haemophilia community, in particular, and in subjects with coagulopathies, in general and the present invention relates to improved compounds capable of substituting for FVIII and thus being useful for the treatment of a coagulopathy such as haemophilia A.

### SUMMARY

The present invention relates to compounds, which serve as a substitute for coagulation Factor VIII (FVIII) in patients suffering from a coagulopathy and in particular patients lacking functional FVIII, such as haemophilia A patients including haemophilia A patients with inhibitors. Hence, one aspect of the present invention relates to compounds capable of enhancing the generation of FXa and thus partially or completely restoring coagulation in patients lacking functional FVIII.

In one aspect, the compound is an antibody or antigen-binding fragment thereof. In one such aspect, the compound is a multispecific antibody or antigen-binding fragment thereof such as a bispecific antibody or antigen-binding fragment thereof.

In one particular aspect, the invention relates to procoagulant antibodies or antigen-binding fragment thereof which serve as a substitute for FVIII in patients lacking functional FVIII, such as haemophilia A patients.

In one such aspect, the antibody or antigen-binding fragment thereof is capable of binding FIX(a) and increases the enzymatic activity of FIXa towards FX, optionally also being capable of binding FX.

In one aspect, the invention relates to a procoagulant antibody or antigen-binding fragment thereof that is capable of binding FIX(a) and FX(a), including bispecific procoagulant antibodies or antigen-binding fragment thereof which increase the enzymatic activity of FIXa towards FX. In one aspect, the invention relates to a procoagulant bispecific antibody or antigen-binding fragment thereof that is capable of binding to FIX(a) and FX(a).

A further aspect of the invention relates to the individual component (intermediate) antibodies or antigen-binding fragment thereof that are part of a procoagulant antibody, such as a particular anti-FIX(a) antibody or antigen-binding fragment thereof or a particular anti-FX(a) antibody or antigen-binding fragment thereof.

A further aspect of the invention relates to the manufacture of the antibodies or antigen-binding fragment thereof—and components (intermediates) thereof—as disclosed herein.

A further aspect of the invention relates to an antibody that competes with a procoagulant antibody or antigen-binding fragment thereof, as disclosed herein, for binding to FIX(a) and/or FX(a).

A further aspect of the invention relates to an antibody or antigen-binding fragment thereof which shares epitope residues or epitope hot-spot residues on FIX(a) and/or FX(a) with a procoagulant antibody or antigen-binding fragment hereof, as disclosed herein.

A further aspect of the invention is directed to the procoagulant antibodies or antigen-binding fragment thereof disclosed herein for prevention and/or treatment of a coagulopathy, a disease accompanying coagulopathy, or a disease caused by coagulopathy. In one aspect the coagulopathy is haemophilia A with or without inhibitors.

A still further aspect of the invention relates to a pharmaceutical composition comprising a procoagulant antibody or antigen-binding fragment thereof as disclosed herein formulated for the delivery of said antibody for the prevention and/or treatment of a coagulopathy, such as haemophilia A with or without inhibitors, as well as an injection device with content thereof.

A further aspect of the invention is directed to a kit comprising (i) an antibody or antigen-binding fragment thereof as disclosed herein such as a bispecific antibody and (ii) instructions for use. The invention may also solve further problems that will be apparent from the disclosure of the exemplary embodiments.

### BRIEF DESCRIPTION OF THE FIGURES

FIG. 1A-1D shows aligments of sequences representing heavy- and light chain variable domains of the anti-FIX(a) (FIGS. 1A and 1B) and anti-FX(a) (FIGS. 10 and 1D) IgG antibodies as disclosed herein. CDR1, 2 and 3 sequences are highlighted in bold and underlined in uppermost sequence and is representative for the remaining sequences.

FIG. 2 shows thrombin generation test (TGT) results from the bispecific antibodies bimAb05-0745, bimAb05-3761, bimAb05-3769, bimAb05-0746, bimAb05-2112, bimAb05-2113, bimAb05-2114 and ACE910 in human tissue factor activated haemophilia A platelet-poor plasma (PPP). The experiment was performed as described in Example 16. Dotted and stippled lines indicate the peak thrombin level (nM) observed in the absence of antibody in HA-PPP and normal PPP, respectively, and with their standard deviation indicated by dotted lines. The profiles of bimAb05-0745, bimAb05-3761, bimAb05-3769, bimAb05-0746, bimAb05-2112, bimAb05-2113 and bimAb05-2114 are indicated by down-pointing triangles, whereas that of ACE910 is indicated by up-pointing triangles. Results are shown as mean±standard deviation from at least three independent experiments.

FIG. 3 shows thrombin generation test (TGT) results from the bispecific antibodies bimAb05-0745, bimAb05-3761, bimAb05-3769, bimAb05-0746, bimAb05-2112, bimAb05-2113, bimAb05-2114 and ACE910 in human tissue factor activated haemophilia A platelet-rich plasma (PRP). The experiment was performed as described in Example 16. Dotted and stippled lines indicate the peak thrombin level (nM) observed in the absence of antibody in HA-PRP and normal PRP, respectively, and with their standard deviation indicated by the dotted lines. The profiles of bimAb05-0745, bimAb05-3761, bimAb05-3769, bimAb05-0746, bimAb05-2112, bimAb05-2113 and bimAb05-2114 are indicated by down-pointing triangles, whereas that of ACE910 is indicated by up-pointing triangles. Results are shown as mean±standard deviation from at least three independent experiments.

FIG. 4A-4B shows results from antibody binning experiments on an Octet Fortebio system using a modified in-tandem setup. Briefly, biotinylated human FIXa was captured on streptavidin tips. Captured FIXa was then saturated by initial exposure to a first (1st) bivalent anti-FIXa antibody, which was followed by a second exposure to an equimolar mixture of the first (1st) antibody and a second (2nd) bivalent anti-FIXa antibody. Binding responses for each of the three phases as well as the identity of the antibodies used are shown. In FIG. 4A, top diagram (labelled as C), the 1^{st} bivalent anti-FIXa antibody is mAb01-2434 and the 2^{nd} bivalent anti-FIXa antibodies are mAb01-2434 (shown in dash line) or mAb01-1767 (shown in solid line), respectively. In FIG. 4A, bottom diagram (labelled as D), the 1^{st} bivalent anti-FIXa antibody is mAb01-2434 and the 2^{nd} bivalent anti-FIXa antibodies are IgG4 (shown in dash line) or mAb01-9985 (shown in solid line), respectively. In FIG. 4B, top diagram (labelled as E), the 1^{st} bivalent anti-FIXa antibody is mAb01-1582 and the 2^{nd} bivalent anti-FIXa antibodies are mAb01-1582 (shown in dash line) or mAb01-1767 (shown in solid line), respectively. In FIG. 4B, bottom diagram (labelled as F), the 1^{st} bivalent anti-FIXa antibody is mAb01-1582 and the 2^{nd} bivalent anti-FIXa antibodies are IgG4 (shown in dash line) or mAb01-9985 (shown in solid line), respectively.

FIG. 5 shows results from antibody binning experiments on an Octet Fortebio system using a modified in-tandem setup. Briefly, biotinylated human FXa was captured on streptavidin tips. Captured FXa was then saturated by initial exposure to a first (1st) bivalent anti-FXa antibody, which was followed by a second exposure to an equimolar mixture of the first (1st) antibody and a second (2nd) bivalent anti-FXa antibody. Binding responses for each of the three phases as well as the identity of the antibodies used are shown. In top diagram (labelled as A), the 1^{st} bivalent anti-FIXa antibody is mAb01-2435 and the 2^{nd} bivalent anti-FIXa antibodies are mAb01-2435 (shown in dash line) or mAb01-6723 (shown in solid line), respectively. In bottom diagram (labelled as B), the 1^{st} bivalent anti-FIXa antibody is mAb01-2435 and the 2^{nd} bivalent anti-FIXa antibodies are IgG4 (shown in dash line) or mAb01-8174 (shown in solid line), respectively.

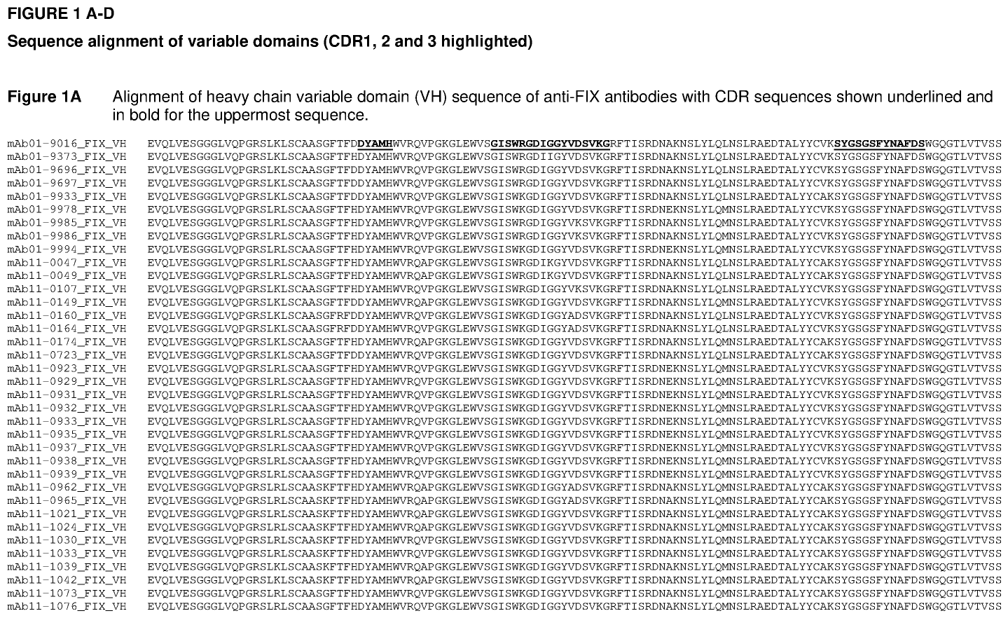

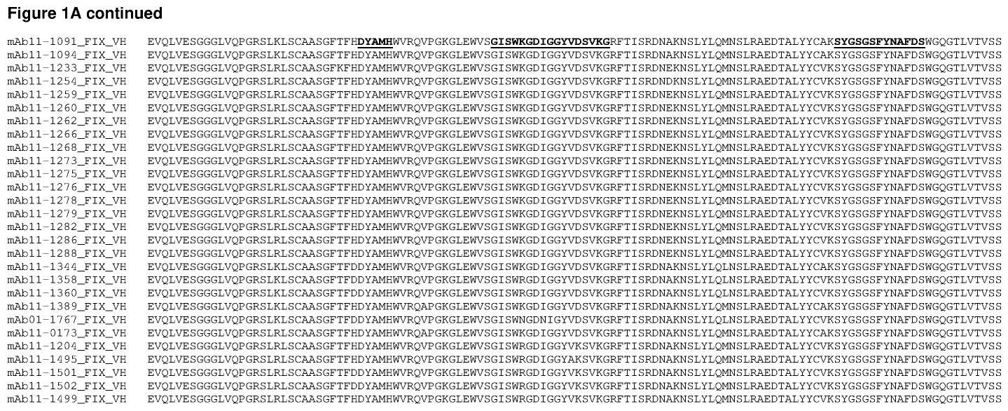

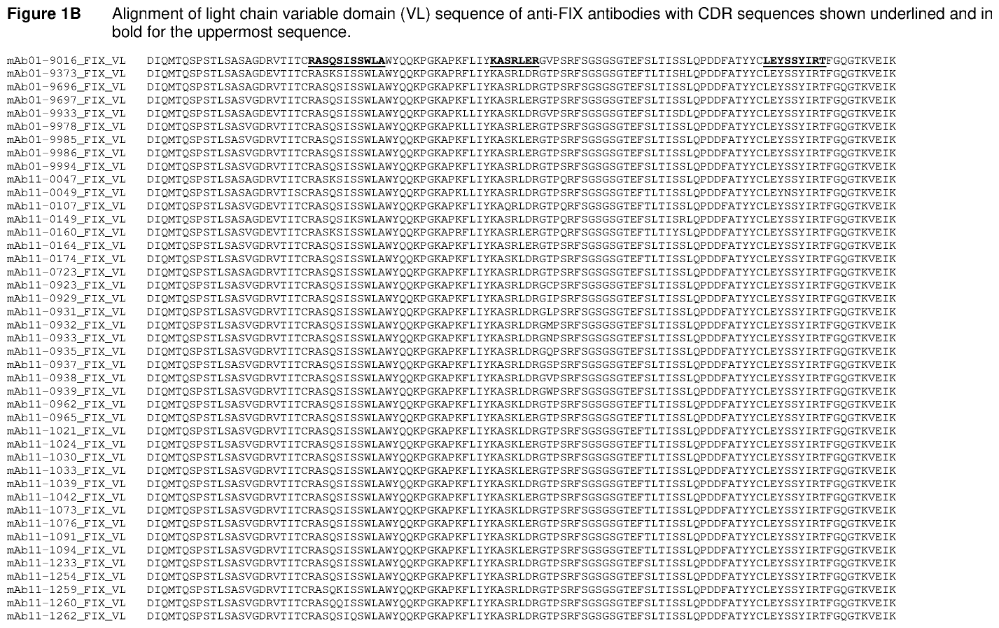

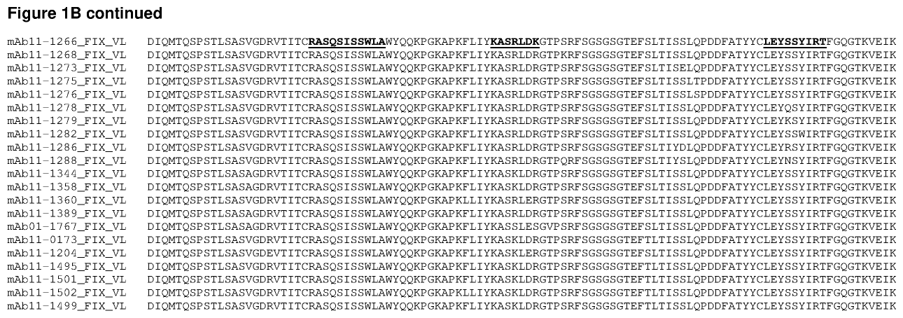

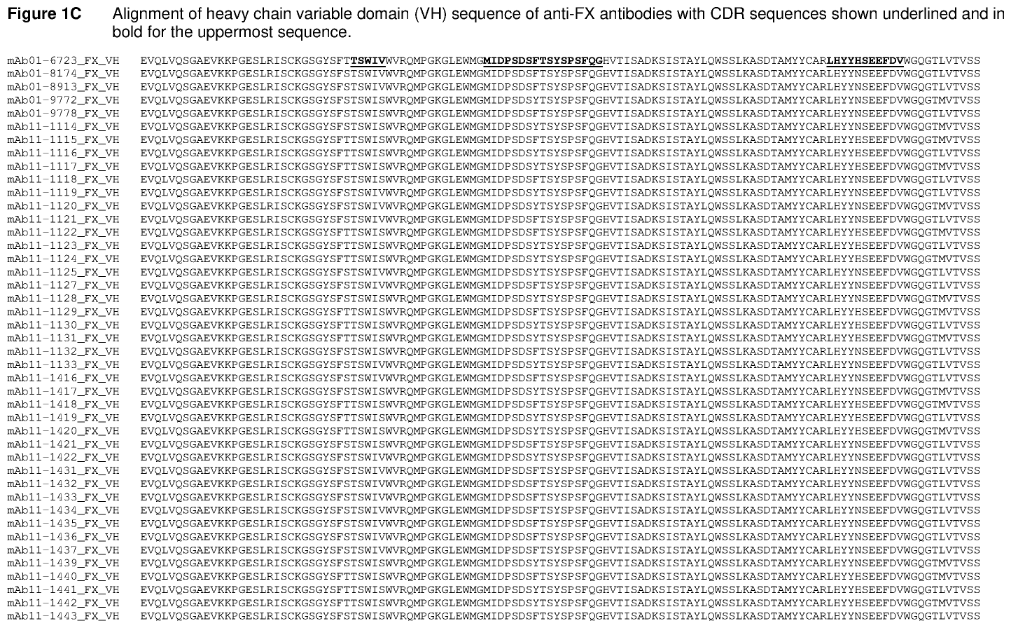

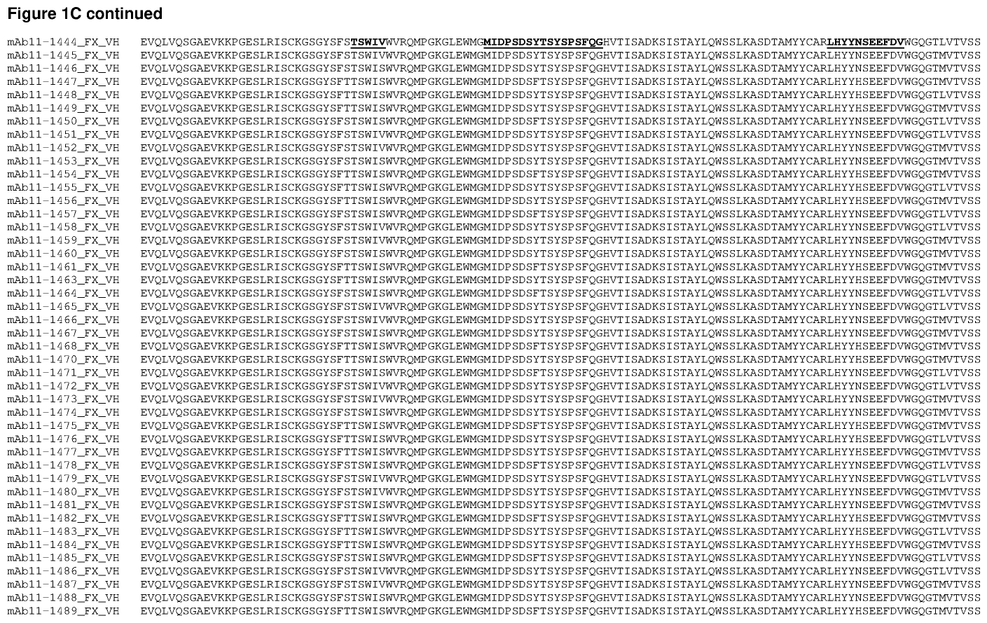

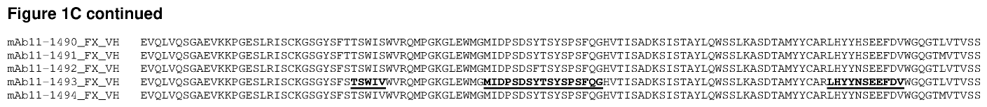

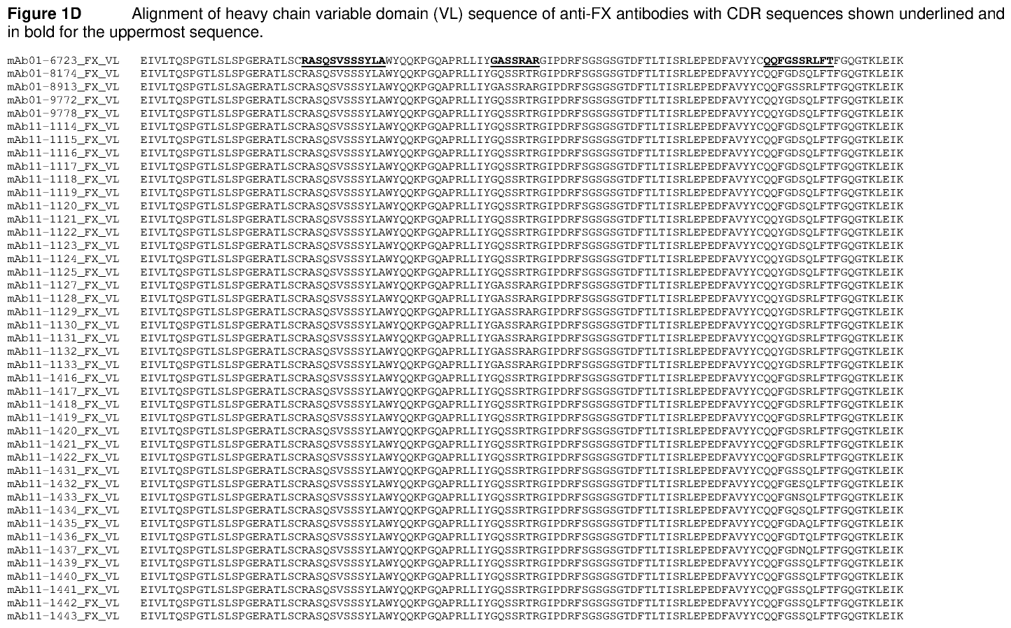

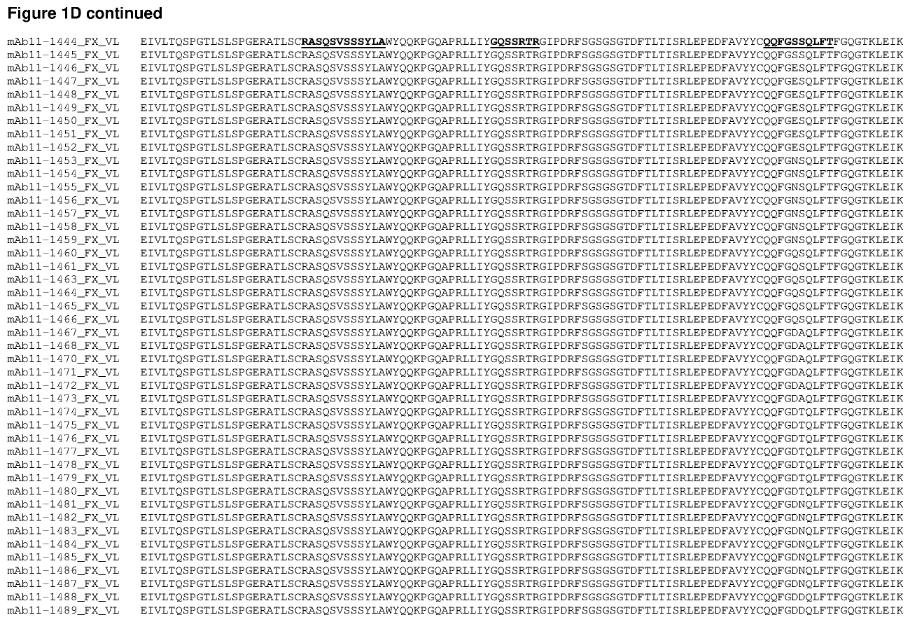

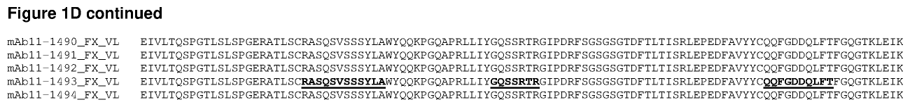

### BRIEF DESCRIPTION OF THE SEQUENCES

SEQ ID NO:1 represents the amino acid sequence of human coagulation Factor IX.

SEQ ID NO:2 represents the amino acid sequence of human coagulation Factor X.

SEQ ID NOs:3-1194 and 1202-1249 represent the sequences of the heavy chain variable domains (VH) and light chain variable domains (VL) and Complementarity Determining Regions (CDRs) of anti-FIX(a) and anti-FX(a) monoclonal antibodies (mAbs) described herein.

SEQ ID NO:1195 represents the human IgG4 heavy chain contant region with S228P and C-terminal lysine truncation.

SEQ ID NO:1196 represents the human IgG4 heavy chain constant region with S228P, F405L, R409K and C-terminal lysine truncation.

SEQ ID NO:1197 represents the human kappa light chain constant region.

SEQ ID NO:1198 represents the human IgG1 heavy chain constant region with F405L and C-terminal lysine truncation.

SEQ ID NO:1199 represents the human IgG1 heavy chain constant region with K405R and C-terminal lysine truncation.

SEQ ID NO:1200 represents a N-terminal His-tag.

SEQ ID NO:1201 represents a GS-linker.

The tables in Example 6 link the SEQ ID NOs to individual (component) anti-FIX(a) and anti-FX(a) antibodies and bispecific antibodies of the invention.

### DESCRIPTION

In subjects with a coagulopathy, such as in human beings with haemophilia A, the coagulation cascade is rendered dysfunctional due to the absence or insufficient presence of functional FVIII. Such dysfunction of one part of the coagulation cascade results in insufficient blood coagulation and potentially life-threatening bleeding, or damage to internal organs, such as the joints. The present invention relates to compounds, which serve as a substitute for coagulation Factor VIII (FVIII) in patients suffering from a coagulopathy and in particular patients lacking functional FVIII, such as haemophilia A patients including haemophilia A patients with inhibitors. In one aspect, such compound is an antibody.

In particular the inventors of the present invention have surprisingly identified antibodies which mimic FVIII cofactor activity with high potency and efficacy. In one particular aspect, the invention relates to procoagulant antibodies which serve as a substitute for FVIII in patients lacking functional FVIII, such as haemophilia A patients. In one such aspect, the procoagulant antibodies bind to and increase the enzymatic activity of coagulation Factor IXa (FIXa) towards coagulation Factor X (FX), optionally also binding FX. In one such aspect the antibodies of the invention are bispecific antibodies capable of binding to FIX/FIXa and FX.

A further aspect of the invention relates to the individual component (intermediate) antibodies or antigen-binding fragment thereof that are part of a multispecific procoagulant antibody, such as a particular anti-FIX(a) antibody or antigen-binding fragment thereof or a particular anti-FX(a) antibody or antigen-binding fragment thereof.

A further aspect of the invention relates to the manufacture of the antibodies or antigen-binding fragment thereof—and components (intermediates) thereof—as disclosed herein.

A further aspect of the invention relates to an antibody that competes with a procoagulant antibody or antigen-binding fragment thereof, as disclosed herein, for binding to FIX(a) and/or FX(a).

A further aspect of the invention relates to an antibody or antigen-binding fragment thereof which shares epitope residues or epitope hot-spot residues on FIX(a) and/or FX(a) with a procoagulant antibody or antigen-binding fragment hereof, as disclosed herein.

In one aspect, the antibody is a human or humanised antibody, such as a human or humanised bispecific antibody.

A further aspect of the invention is directed to the procoagulant antibodies or antigen-binding fragment thereof disclosed herein for prevention and/or treatment of a coagulopathy, a disease accompanying coagulopathy, or a disease caused by coagulopathy. In one aspect the coagulopathy is haemophilia A with or without inhibitors.

A still further aspect of the invention relates to a pharmaceutical composition comprising a procoagulant antibody or antigen-binding fragment thereof as disclosed herein formulated for the delivery of said antibody for the prevention and/or treatment of a coagulopathy, such as haemophilia A with or without inhibitors, as well as an injection device with content thereof.

A further aspect of the invention is directed to a kit comprising (i) an antibody or antigen-binding fragment thereof as disclosed herein such as a bispecific antibody and (ii) instructions for use.

Coagulation Factor IX

Coagulation Factor IX (FIX) is a vitamin K-dependent coagulation factor with structural similarities to Factor VII, prothrombin, Factor X, and Protein C. FIX circulates in plasma as a single-chain zymogen (SEQ ID NO:1). The circulating zymogen form consists of 415 amino acids divided into four distinct domains comprising an N-terminal γ-carboxyglutamic acid-rich (Gla) domain, two EGF domains and a C-terminal trypsin-like serine protease domain. Activation of FIX occurs by limited proteolysis at Arg145 and Arg180 to release the activation peptide (residues 146 to 180 of SEQ ID NO:1). Thus, activated FIX (FIXa) is composed of residues 1-145 of SEQ ID NO:1 (light chain) and residues 181-415 of SEQ ID NO:1 (heavy chain). Circulating FIX molecules thus comprise the FIX zymogen and the activated form of FIX which are herein generally referred to as FIX and FIXa with reference to SEQ ID NO:1.

Activated Factor IX is referred to as Factor IXa or FIXa. The term “FIX (SEQ ID NO:1) and/or the activated form thereof (FIXa)” may also be referred to as “FIX/FIXa” or simply “FIX(a)”. FIXa is a trypsin-like serine protease that serves a key role in haemostasis by generating, as part of the tenase complex, most of the Factor Xa required to support proper thrombin formation during coagulation.

FIX is herein represented by SEQ ID NO:1 corresponding to the Ala148 allelic form of human FIX (Anson et al. EMBO J. 1984 3:1053-1060; McGraw et al., Proc Natl Acad Sci USA. 1985 82:2847-2851; Graham et al. Am. J. Hum. Genet. 1988 42:573-580). In the present invention FIX is intended to cover all natural variants of FIX, such as the T148 variant (Uniprot ID P00740).

Coagulation Factor X

FX is a vitamin K-dependent coagulation factor with structural similarities to Factor VII, prothrombin, FIX, and protein C. FX circulates in plasma as a two-chain zymogen including residues 1-139 of SEQ ID NO:2 (light chain) and residues 143-448 of SEQ ID NO:2 (heavy chain). Human FX zymogen comprises four distinct domains comprising an N-terminal gamma-carboxyglutamic acid rich (Gla) domain (residues 1-45), two EGF domains, EGF1 (residues 46-82) and EGF2 (residues 85-125), respectively, and a C-terminal trypsin-like serine protease domain (residues 195-448). Activation of FX occurs by limited proteolysis at Arg194, which results in the release of the activation peptide (residues 143-194). Thus, activated FX (FXa) is composed of residues 1-139 of SEQ ID NO:2 (light chain) and residues 195-448 of SEQ ID NO:2 (activated heavy chain). Circulating Factor X molecules thus comprises the FX zymogen and the activated form of FX which are herein referred to as FX and FXa, respectively, with reference to SEQ ID NO:2. In the present invention FX is intended to cover all natural variants of FX. The term “FX (SEQ ID NO:2) and/or the activated form thereof (FXa)” may also be referred to as “FX/FXa” or “FX(a)”.

Antibodies

The term “antibody” herein refers to a protein, derived from an immunoglobulin sequence, which is capable of binding to an antigen or a portion thereof. The term antibody includes, but is not limited to, full length antibodies of any class (or isotype), that is, IgA, IgD, IgE, IgG, IgM and/or IgY. The term antibody includes—but is not limited to—antibodies that are bivalent, such as bispecific antibodies.

Natural full-length antibodies comprise at least four polypeptide chains: two heavy chains (HC) and two light chains (LC) that are connected by disulfide bonds. In some cases, natural antibodies comprise less than four chains, as in the case of the IgNARs found in Chondrichthyes. One class of immunoglobulins of particular pharmaceutical interest is the IgGs. In humans, the IgG class may be divided into four sub-classes IgG1, IgG2, IgG3 and IgG4, based on the sequence of their heavy chain constant regions. The light chains can be divided into two types, kappa and lambda chains, based on differences in their sequence composition. IgG molecules are composed of two heavy chains, interlinked by two or more disulfide bonds, and two light chains, each attached to a heavy chain by a disulfide bond. An IgG heavy chain may comprise a heavy chain variable domain (V_{H}) and up to three heavy chain constant (C_{H}) domains: C_{H}1, C_{H}2 and C_{H}3. A light chain may comprise a light chain variable domain (V_{L}) and a light chain constant domain (C_{L}). V_{H} and V_{L} regions can be further subdivided into regions of hypervariability, termed complementarity determining regions (CDRs) or hypervariable regions (HvRs), interspersed with regions that are more conserved, termed framework regions (FR). V_{H} and V_{L} domains are typically composed of three CDRs and four FRs, arranged from amino-terminus to carboxy-terminus in the following order: FR1, CDR1, FR2, CDR2, FR3, CDR3, FR4. The heavy and light chain variable domains containing the hypervariable regions (CDRs) form a structure that is capable of interacting with an antigen, whilst the constant region of an antibody may mediate binding of the immunoglobulin to host tissues or factors, including, but not limited to various cells of the immune system (effector cells), Fc receptors and the first component, C1q, of the C1 complex of the classical complement system.

Antibodies of the invention may be monoclonal antibodies (mAbs), in the sense that they represent a set of unique heavy and light chain variable domain sequences as expressed from a single B-cell or by a clonal population of B cells. Antibodies of the invention may be produced and purified using various methods that are known to a person skilled in the art. For example, antibodies may be produced from hybridoma cells. Antibodies may be produced by B-cell expansion. Antibodies or fragment thereof may be recombinantly expressed in mammalian or microbial expression systems, or by in vitro translation. Antibodies or fragment thereof may also be recombinantly expressed as cell surface bound molecules, by means of e.g. phage display, bacterial display, yeast display, mammalian cell display or ribosome or mRNA display. Antibodies of the current invention may be isolated. The term “isolated antibody” refers to an antibody that has been separated and/or recovered from (an)other component(s) in the environment in which it was produced and/or that has been purified from a mixture of components present in the environment in which it was produced.

Certain antigen-binding fragments of antibodies may be suitable in the context of the current invention, as it has been shown that the antigen-binding function of an antibody can be performed by fragments of a full-length antibody. The term “antigen-binding fragment” of an antibody refers to one or more fragment(s) of an antibody that retain(s) the ability to specifically bind to or recognise an antigen, such as FIX/FIXa, FX/FXa or another target molecule, as described herein. Examples of antigen-binding fragments include (but is not limited to) Fab, Fab′, Fab₂, Fab′₂, Fv (typically the combination of V_{L} and V_{H} domains of a single arm of an antibody), single-chain Fv (scFv); see e.g. Bird et al. Science 1988; 242:423-426; and Huston et al. PNAS 1988; 85:5879-5883), dsFv, Fd (typically the V_{H} and C_{H}1 domain), monovalent molecules comprising both a single V_{H} and a single V_{L} domain; minibodies, diabodies, triabodies, tetrabodies, and kappa bodies (see, e.g. III et al (1997) Protein Eng 10: 949-57); as well as one or more isolated CDRs or a functional paratope, where the isolated CDRs or antigen-binding residues or polypeptides can be associated or linked together so as to form a functional antibody fragment. These antibody fragments may be obtained using conventional techniques known to those skilled in the art, and the fragments may be screened for utility in the same manner as intact antibodies.

“Fab fragments” of an antibody, including “Fab” and “Fab′₂” fragments, can be derived from an antibody by cleavage of the heavy chain in the hinge region on the N-terminal or C-terminal side, respectively, of the hinge cysteine residues connecting the heavy chains of the antibody. A “Fab” fragment includes the variable and constant domains of the light chain and the variable domain and C_{H}1 domain of the heavy chain. “Fab′₂” fragments comprise a pair of “Fab′” fragments that are generally covalently linked by their hinge cysteines. A Fab′ is formally derived from a Fab′₂ fragment by cleavage of the hinge disulfide bonds connecting the heavy chains in the Fab′₂. Other chemical couplings than disulfide linkages of antibody fragments are also known in the art. A Fab fragment retains the ability of the parent antibody to bind to its antigen, potentially with a lower affinity. Fab′₂ fragments are capable of bivalent binding, whereas Fab and Fab′ fragments can only bind monovalently. Generally, Fab fragments lack the constant C_{H}2 and C_{H}3 domains, i.e. the Fc part, where interaction with the Fc receptors and C1q would occur. Thus, Fab fragments are in general devoid of effector functions. Fab fragments may be produced by methods known in the art, either by enzymatic cleavage of an antibody, e.g. using papain to obtain the Fab or pepsin to obtain the Fab′₂, Fab fragments including Fab, Fab′, Fab′₂ may be produced recombinantly using techniques that are well known to the person skilled in the art. An “Fv” (fragment variable) fragment is an antibody fragment that contains a complete antigen recognition and binding site, and generally comprises one heavy and one light chain variable domain in association that can be covalent in nature, for example in a single chain variable domain fragment (scFv). It is in this configuration that the three hypervariable regions of each variable domain interact to define an antigen-binding site on the surface of the V_{H}-V_{L} dimer. Collectively, the six hypervariable regions or a subset thereof confer antigen binding specificity to the antibody.

“Single-chain Fv” or “scFv” antibody comprise the V_{H} and V_{L} domains of antibody, where these domains are present in a single polypeptide chain. Generally, the Fv polypeptide further comprises a polypeptide linker between the V_{H} and V_{L} domains that enables the scFv to form the desired structure for antigen binding. For a review of scFv, see Pluckthun, 1994, In: The Pharmacology of Monoclonal Antibodies, Vol. 113, Rosenburg and Moore eds. Springer-Verlag, New York, pp. 269-315.

“Single-chain Fab” or “scFab” antibody comprise the V_{H}, C_{H}1, V_{L} and C_{L} domains of an antibody, where these domains are present in a single polypeptide chain. Generally, the Fab polypeptide further comprises a polypeptide linker between either V_{H} and C_{L} or V_{L} and C_{H}1 domains that enables the scFab to form the desired structure for antigen binding (Koerber et al. (2015) J Mol Biol. 427:576-86).

The term “diabodies” refers to small antibody fragments with two antigen-binding sites, in which fragments comprise a heavy chain variable domain (V_{H}) connected to a light chain variable domain (V_{L}) in the same polypeptide chain (V_{H} and V_{L}). By using a linker that is too short to allow pairing between the two variable domains on the same chain, the variable domains are forced to pair with complementary domains of another chain, creating two antigen-binding sites.

The expression “linear antibodies” refers to antibodies as described in Zapata et al. (1995) Protein Eng. 8: 1057-1062. Briefly, these antibodies contain a pair of tandem Fd segments (V_{H}-C_{H}1-V_{H}-C_{H}1) that, together with complementary light chain polypeptides, form a pair of antigen binding regions. Linear antibodies can be bispecific or monospecific.

Antibody fragments may be obtained using conventional recombinant or protein engineering techniques and the fragments can be screened for binding to FIX and the activated form thereof, FX or another function, in the same manner as intact antibodies.

Antibody fragments of the invention may be made by truncation, e.g. by removal of one or more amino acids from the N and/or C-terminal ends of a polypeptide. Fragments may also be generated by one or more internal deletions.

An antibody of the invention may be, or may comprise, a fragment of the antibody, or a variant of any one of the antibodies disclosed herein. An antibody of the invention may be, or may comprise, an antigen binding portion of one of these antibodies, or variants thereof. For example, an antibody of the invention may be a Fab fragment of one of these antibodies or variants thereof, or it may be a single chain antibody derived from one of these antibodies, or a variant thereof. Also, an antibody of the invention may be a combination of a full length antibody and fragment thereof.

The term “one-armed” as used herein, refers to a particular type of monovalent antibody constituted by an antibody heavy chain, a truncated heavy chain lacking the Fab region, and a single light chain.

The term “monospecific” antibody as used herein, refers to an antibody which is capable of binding to one particular epitope (including but not limited to bivalent antibodies).

The term “bispecific” antibody as used herein, refers to an antibody which is capable of binding to two different antigens or two different epitopes on the same antigen.

The term “trispecific” antibody as used herein, refers to an antibody which is capable of binding to three different antigens or three different epitopes on the same antigen or three different epitopes present on two different antigens.

The term “multispecific” antibody as used herein, refers to an antibody which is capable of binding to two or more different antigens or two or more different epitopes on the same antigen. Multispecific antibodies thus comprise bi- and trispecific antibodies.

Bispecific antibodies in full length IgG format can be generated by fusion of two individual hybridomas to form a hybrid quadroma which produces a mixture of antibodies including a fraction of bispecific heterodimerising antibodies (Chelius D. et al.; MAbs. 2010 May-June; 2(3): 309-319). Bispecific heterodimerising antibodies may alternatively be produced by using recombinant technologies. Heterodimerisation can also be achieved by engineering the dimerisation interface of the Fc region to promote heterodimerisation. One example hereof is the so-called knob-in-hole mutations where sterically bulky side chains (knobs) are introduced in one Fc matched by sterically small side chains (holes) on the opposite Fc thereby creating steric complementarity promoting heterodimerisation. Other methods for engineered heterodimerisation Fc interfaces are electrostatic complementarity, fusion to non-IgG heterodimerisation domains or utilising the natural Fab-arm exchange phenomenon of human IgG4 to control heterodimerisation. Examples of heterodimerised bispecific antibodies are well described in the literature, e.g. (Klein C, et al.; MAbs. 2012 November-December; 4(6): 653-663). Special attention has to be paid to the light chains in heterodimeric antibodies. Correct pairing of LCs and HCs can be accomplished by the use of a common light chain. Again engineering of the LC/HC interface can be used to promote heterodimerisation or light chain cross-over engineering as in CrossMabs. In vitro re-assembly under mildly reducing conditions of antibodies from two individual IgGs containing appropriate mutations can also be used to generate bispecific antibodies (e.g. Labrijn et al., PNAS, 110, 5145-5150 (2013)). Also the natural Fab-arm exchange method is reported to ensure correct light chains paring.

Multispecific antibody-based molecules may also be expressed recombinantly as fusion proteins combining the natural modules of IgGs to form multispecific and multivalent antibody derivatives as described in the literature. Examples of fusion antibodies are DVD-Igs, IgG-scFV, Diabodies, DARTs etc. Specific detection or purification tags, half-life extension moieties or other components can be incorporated in the fusion proteins. Additional non-IgG modalities may also be incorporated in the fusion proteins. Bispecific full length antibodies based on Fc heterodimerisation are commonly referred to as asymmetic IgGs, irrespective of the LC paring methodology.

Generally, bispecific antibodies may be produced in a variety of molecular formats as reviewed by Brinkmann et al. (Brinkmann et al. The making of bispecific antibodies. Mabs 9, 182-212 (2017)).

Multispecific antibody-based molecules may also be produced by chemical conjugation or coupling of individual full length IgGs or coupling of fragments of IgGs to form multispecific and multivalent antibody derivatives as described in the literature. Examples are chemically coupled Fab fragments, IgG-dimer etc. Specific detection or purification tags, half-life extension molecules or other components can be incorporated in the conjugate proteins. Additional non-IgG polypeptide may also be incorporated in the fusion proteins. Multispecific molecules may also be produced by combining recombinant and chemical methods including those described above.

In one aspect, an antibody of the invention is a chimeric antibody, a human antibody or a humanised antibody. Such antibody can be generated by using, for example, suitable antibody display or immunization platforms or other suitable platforms or methods known in the field. The term “human antibody”, as used herein, is intended to include antibodies having variable domains in which at least a portion of a framework region and/or at least a portion of a CDR region are derived from human germline immunoglobulin sequences. For example, a human antibody may have variable domains in which both the framework and CDR regions are derived from human germline immunoglobulin sequences. Furthermore, if the antibody contains a constant region, the constant region or a portion thereof is also derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo). Such a human antibody may be a human monoclonal antibody. Such a human monoclonal antibody may be produced by a hybridoma which includes a B cell obtained from a transgenic nonhuman animal, e.g., a transgenic mouse, having a genome comprising human immunoglobulin heavy and light chain gene segments repertoires, fused to an immortalised cell. Human antibodies may be isolated from sequence libraries built on selections of human germline sequences, further diversified with natural and synthetic sequence diversity.

Human antibodies may be prepared by in vitro immunisation of human lymphocytes followed by transformation of the lymphocytes with Epstein-Barr virus.

Human antibodies may be produced by recombinant methods known in the art.

The term “human antibody derivative” refers to any modified form of the human antibody, such as a conjugate of the antibody and another agent or antibody.

The term “humanised antibody”, as used herein, refers to a human/non-human antibody that contains a sequence (CDR regions or parts thereof) derived from a non-human immunoglobulin. A humanised antibody is, thus, a human immunoglobulin (recipient antibody) in which residues from at least a hypervariable region of the recipient are replaced by residues from a hypervariable region of an antibody from a non-human species (donor antibody) such as from a mouse, rat, rabbit or non-human primate, which have the desired specificity, affinity, sequence composition and functionality. In some instances, framework (FR) residues of the human immunoglobulin are replaced by corresponding non-human residues. An example of such a modification is the introduction of one or more so-called back-mutations, which are typically amino acid residues derived from the donor antibody. Humanisation of an antibody may be carried out using recombinant techniques known to the person skilled in the art (see, e.g., Antibody Engineering, Methods in Molecular Biology, vol. 248, edited by Benny K. Lo). A suitable human recipient framework for both the light and heavy chain variable domain may be identified by, for example, sequence or structural homology. Alternatively, fixed recipient frameworks may be used, e.g., based on knowledge of structure, biophysical and biochemical properties. The recipient frameworks can be germline derived or derived from a mature antibody sequence. CDR regions from the donor antibody can be transferred by CDR grafting. The CDR grafted humanised antibody can be further optimised for e.g. affinity, functionality and biophysical properties by identification of critical framework positions where re-introduction (back-mutation) of the amino acid residue from the donor antibody has beneficial impact on the properties of the humanised antibody. In addition to donor antibody derived back-mutations, the humanised antibody can be engineered by introduction of germline residues in the CDR or framework regions, elimination of immunogenic epitopes, site-directed mutagenesis, affinity maturation, etc.

Furthermore, humanised antibodies may comprise residues that are not found in the recipient antibody or in the donor antibody. These modifications are made to further refine antibody performance. In general, a humanised antibody will comprise at least one—typically two—variable domains, in which all or substantially all of the CDR regions correspond to those of a non-human immunoglobulin and in which all or substantially all of the FR residues are those of a human immunoglobulin sequence. The humanised antibody can, optionally, also comprise at least a portion of an immunoglobulin constant region (Fc), typically that of a human immunoglobulin.

The term “humanised antibody derivative” refers to any modified form of the humanised antibody, such as a conjugate of the antibody and a chemical agent or a conjugate of the antibody with another antibody.

The term “chimeric antibody”, as used herein, refers to an antibody comprising portions of antibodies derived from two or more species. For example, the genes encoding such antibody comprise genes encoding variable domains and genes encoding constant domains originated from two different species. For example, the genes encoding variable domains of a mouse monoclonal antibody may be joined to the genes encoding the constant domains of an antibody of human origin.

The fragment crystallisable region (“Fc region”/“Fc domain”) of an antibody is the C-terminal region of an antibody, which comprises the hinge and the constant C_{H}2 and C_{H}3 domains. The Fc domain may interact with cell surface receptors called Fc receptors, as well as some proteins of the complement system. The Fc region enables antibodies to interact with the immune system. In one aspect of the invention, antibodies may be engineered to include modifications within the Fc region, typically to alter one or more of its functional properties, such as serum half-life, complement fixation, Fc-receptor binding, protein stability and/or antigen-dependent cellular cytotoxicity, or lack thereof, among others. Furthermore, an antibody of the invention may be chemically modified (e.g., one or more chemical moieties can be attached to the antibody) or be modified to alter its glycosylation, again to alter one or more functional properties of the antibody. An IgG1 antibody may carry a modified Fc domain comprising one or more, and perhaps all of the following mutations that will result in decreased affinity to certain Fc-gamma receptors (L234A, L235E, and G237A) and in reduced C1q-mediated complement fixation (A330S and P331S), respectively (residue numbering according to the EU index). Alternatively, other amino acid substitutions, and combinations thereof and combinations with the above mentioned, known in the art to lead to altered (reduced or increased) Fc-gamma receptor binding may be used.

The isotype of an antibody of the invention may be IgG, such as IgG1, such as IgG2, such as IgG4. If desired, the class of an antibody may be “switched” by known techniques. For example, an antibody that was originally produced as an IgM molecule may be class switched to an IgG antibody. Class switching techniques also may be used to convert one IgG subclass to another, for example: from IgG1 to IgG2 or IgG4; from IgG2 to IgG1 or IgG4; or from IgG4 to IgG1 or IgG2. Engineering of antibodies to generate constant region chimeric molecules, by combination of regions from different IgG subclasses, can also be performed.

In one embodiment the hinge region of the antibody is modified such that the number of cysteine residues in the hinge region is altered, e.g., increased or decreased. This approach is described further for instance in U.S. Pat. No. 5,677,425 by Bodmer et al.

The constant region may be modified to stabilise the antibody, e.g., to reduce the risk of a bivalent antibody separating into half antibodies. For example, in an IgG4 constant region, residue S228 (according to the EU numbering index and S241 according to Kabat) may be mutated to a proline (P) residue to stabilise inter heavy chain disulphide bridge formation at the hinge (see, e.g., Angal et al. Mol Immunol. 1993; 30:105-8).

Antibodies or fragment thereof may be defined in terms of their complementarity-determining regions (CDRs). The term “complementarity-determining region” or “hypervariable region”, when used herein, refers to the regions of an antibody in which amino acid residues involved in antigen-binding are situated. The region of hypervariability or CDRs can be identified as the regions with the highest variability in amino acid alignments of antibody variable domains. Databases can be used for CDR identification such as the Kabat database, the CDRs e.g. being defined as comprising amino acid residues 24-34 (L1), 50-56 (L2) and 89-97 (L3) of the light-chain variable domain and 31-35 (H1), 50-65 (H2) and 95-102 (H3) in the heavy-chain variable domain; (Kabat et al. 1991; Sequences of Proteins of Immunological Interest, Fifth Edition, U.S. Department of Health and Human Services, NIH Publication No. 91-3242). Alternatively CDRs can be defined as those residues from a “hypervariable loop” (residues 26-33 (L1), 50-52 (L2) and 91-96 (L3) in the light-chain variable domain and 26-32 (H1), 53-55 (H2) and 96-101 (H3) in the heavy-chain variable domain; Chothia and Lesk, J. Mol. Biol. 1987; 196:901-917). Typically, the numbering of amino acid residues in this region is performed by the method described in Kabat et al. supra. Phrases such as “Kabat position”, “Kabat residue”, and “according to Kabat” herein refer to this numbering system for heavy chain variable domains or light chain variable domains. Using the Kabat numbering system, the actual linear amino acid sequence of a peptide may contain fewer or additional amino acids corresponding to a shortening of, or insertion into, a framework (FR) or CDR of the variable domain. For example, a heavy chain variable domain may include amino acid insertions (residue 52a, 52b and 52c according to Kabat) after residue 52 of CDR H2 and inserted residues (e.g. residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence.

The term “framework region” or “FR” residues refer to those V_{H} or V_{L} amino acid residues that are not within the CDRs, as defined herein.

An antibody of the invention may comprise a CDR region from one or more of the specific antibodies disclosed herein.

The term “procoagulant antibody” refers to an antibody which potentiates blood coagulation for example by accelerating the process of blood coagulation and/or increasing the enzymatic activity of one or more coagulation factors.

The term “procoagulant activity” refers to the ability of a compound, such as an antibody, to potentiate blood coagulation for example by accelerating the process of blood coagulation and/or increasing the enzymatic activity of one or more coagulation factors.

The term “antigen” (Ag) refers to the molecular entity used for immunisation of an immunocompetent vertebrate to produce the antibody (Ab) that recognizes the Ag. Herein, Ag is termed more broadly and is generally intended to include target molecules that are specifically recognized by the Ab, thus including fragments or mimics of the molecule used in the immunisation process, or other process, e.g. phage display, used for generating the Ab.

The present invention encompasses variants of the antibodies, or antigen-binding fragments thereof of the invention, which may comprise 1, 2, 3, 4 or 5 amino acid substitutions and/or deletions and/or insertions in the individual sequences disclosed herein.

“Substitution” variants preferably involve the replacement of one or more amino acid(s) with the same number of amino acid(s). Substitutions may be, but are not limited to, conservative substitutions. For example, an amino acid may be substituted to an amino acid with similar biochemical properties, for exampe, a basic amino acid may be substituted to another basic amino acid (e.g. lysine to arginine), an acidic amino acid may be substituted to another acidic amino acid (e.g glutamate to aspartate), a neutral amino acid may be substituted to another neutral amino acid (e.g threonine to serine), a charged amino acid may be substituted to another charged amino acid (e.g. glutamate to aspartate), a hydrophilic amino acid may be substituted to another hydrophilic amino acid (e.g. asparagine to glutamine), a hydrophobic amino acid may be substituted to another hydrophobic amino acid (e.g. alanine to valine), a polar amino acid may be substituted to another polar amino acid (e.g. serine to threonine), an aromatic amino acid may be substituted to another aromatic amino acid (e.g. phenylalanine to tryptophan) and an aliphatic amino acid may be substituted to another aliphatic amino acid (e.g. leucine to isoleucine).

Preferred variants include those in which instead of the amino acid which appears in the sequence comprises a structural analog of the amino acid.

Epitope and Paratope

The term “epitope”, as used herein, is defined in the context of a molecular interaction between an “antigen binding polypeptide”, such as an antibody (Ab), and its corresponding antigen (Ag). Generally, “epitope” refers to the area or region on an Ag to which an Ab binds, i.e. the area or region in physical contact with the Ab. Physical contact may be defined using various criteria (e.g. a distance cut-off of 2-6 Å, such as 3 Å, such as 3.5 Å such as 4 Å, such as 4.5 Å, such as 5 Å; or solvent accessibility) for atoms in the Ab and Ag molecules.

FIX/FIXa and FX/FXa may comprise a number of different epitopes, which may include, without limitation, (1) linear peptide epitopes (2) conformational epitopes which consist of one or more non-contiguous amino acids located near each other in the mature FIX/FIXa or FX/FXa conformation; and (3) epitopes which consist, either in whole or part, of molecular structures covalently attached to FIX/FIXa or FX/FXa, such as carbohydrate groups.

The epitope for a given antibody (Ab)/antigen (Ag) pair can be described and characterized at different levels of detail using a variety of experimental and computational epitope mapping methods. The experimental methods include mutagenesis, X-ray crystallography, Nuclear Magnetic Resonance (NMR) spectroscopy, Hydrogen Deuterium eXchange Mass Spectrometry (HDX-MS) and various competition binding methods; methods that are known in the art. As each method relies on a unique principle, the description of an epitope is intimately linked to the method by which it has been determined. Thus, depending on the epitope mapping method employed, the epitope for a given Ab/Ag pair may be described differently.

In the context of an X-ray derived crystal structure defined by spatial coordinates of a complex between an Ab, e.g. a Fab fragment, and its Ag, the term epitope is herein, unless otherwise specified or contradicted by context, specifically defined as FIX/FIXa or FX/FXa residues characterized by having a heavy atom (i.e. a non-hydrogen atom) within a distance of 3.5 Å, from a heavy atom in the Ab.

Epitopes described at the amino acid level, e.g. determined from an X-ray structure, are said to be identical if they contain the same set of amino acid residues. Epitopes are said to overlap if at least one amino acid residue is shared by the epitopes. Epitopes are said to be separate (unique) if no amino acid residue is shared by the epitopes.

The definition of the term “paratope” is derived from the above definition of “epitope” by reversing the perspective. Thus, the term “paratope” refers to the area or region on the antibody, or fragment thereof to which an antigen binds, i.e. to which it makes physical contact to the antigen.

In the context of an X-ray derived crystal structure, defined by spatial coordinates of a complex between an Ab, such as a Fab fragment, and its Ag, the term paratope is herein, unless otherwise specified or contradicted by context, specifically defined as Ab residues characterized by having a heavy atom (i.e. a non-hydrogen atom) within a distance of 3.5 Å from a heavy atom in FIX/FIXa or FX/FXa.

The epitope and paratope for a given antibody (Ab)/antigen (Ag) pair may be identified by routine methods. For example, the general location of an epitope may be determined by assessing the ability of an antibody to bind to different fragments or variants of FIX/FIXa or FX/FXa. The specific amino acids within FIX/FIXa or FX/FXa that make contact with an antibody (epitope) and the specific amino acids in an antibody that make contact with FIX/FIXa or FX/FXa (paratope) may also be determined using routine methods. For example, the antibody and target molecule may be combined and the Ab:Ag complex may be crystallised. The crystal structure of the complex may be determined and used to identify specific sites of interaction between the antibody and its target.

Epitopes on an antigen may comprise one or more hot-spot residues, i.e. residues which are particularly important for the interaction with the cognate antibody, and where interactions mediated by the side chain of said hot-spot residue contribute significantly to the binding energy for the antibody/antigen interaction (Peng et al. (2014) PNAS 111, E2656-E2665). Hot-spot residues can be identified by testing variants of the antigen (here FIX/FIXa and FX), where single epitope residues have been substituted by e.g. alanine, for binding to the cognate antibody. If substitution of an epitope residue with alanine has a strong impact on binding to the antibody, said epitope residue is considered a hot-spot residue, and therefore of particular importance for binding of the antibody to the antigen.

Antibodies that bind to the same antigen can be characterised with respect to their ability to bind to their common antigen simultaneously and may be subjected to “competition binding”/“binning”. In the present context, the term “binning” refers to a method of grouping antibodies that bind to the same antigen. “Binning” of antibodies may be based on competition binding of two antibodies to their common antigen in assays based on standard techniques.

An antibody's “bin” is defined using a reference antibody. If a second antibody is unable to bind to an antigen at the same time as the reference antibody, the second antibody is said to belong to the same “bin” as the reference antibody. In this case, the reference and the second antibody competitively bind the same part of an antigen and are coined “competing antibodies”. If a second antibody is capable of binding to an antigen at the same time as the reference antibody, the second antibody is said to belong to a separate “bin”. In this case, the reference and the second antibody do not competitively bind the same part of an antigen and are coined “non-competing antibodies”.

Antibody “binning” does not provide direct information about the epitope.

Competing antibodies, i.e. antibodies belonging to the same “bin” may have identical epitopes, overlapping epitopes or even separate epitopes. The latter is the case if the reference antibody bound to its epitope on the antigen takes up the space required for the second antibody to contact its epitope on the antigen (“steric hindrance”). Non-competing antibodies generally have separate epitopes. Thus, in some embodiments antibodies of the invention will bind to the same epitope as at least one of the antibodies specifically disclosed herein.

Competition assays for determining whether an antibody competes for binding with, an anti-FIX/FIXa or anti-FX/FXa antibody disclosed herein are known in the art. Exemplary competition assays include immunoassays (e.g., ELISA assays, RIA assays), surface plasmon resonance analysis (e.g. using a BIAcore™ instrument), biolayer interferometry (ForteBio®) and flow cytometry.

Typically, a competition assay involves the use of an antigen bound to a solid surface or expressed on a cell surface, a test FIX- or FIXa binding antibody and a reference antibody. The reference antibody is labelled and the test antibody is unlabelled. Competitive inhibition is measured by determining the amount of labelled reference antibody bound to the solid surface or cells in the presence of the test antibody. Usually the test antibody is present in excess (e.g., 1, 5, 10, 20, 100, 1000, 10000 or 100000 fold). Antibodies identified as being competitive in the competition assay (i.e., competing antibodies) include antibodies binding to the same epitope, or overlapping epitopes, as the reference antibody, and antibodies binding to an adjacent epitope sufficiently proximal to the epitope bound by the reference antibody for steric hindrance to occur. In an exemplary competition assay, a reference anti-FIX or anti-FIXa antibody is biotinylated using commercially available reagents. The biotinylated reference antibody is mixed with serial dilutions of the test antibody or unlabelled reference antibody (self-competition control) resulting in a mixture of various molar ratios (e.g., 1, 5, 10, 20, 100, 1000, 10000 or 100000 fold) of test antibody (or unlabelled reference antibody) to labelled reference antibody. The antibody mixture is added to a FIX or FIXa polypeptide coated-ELISA plate. The plate is then washed, and horseradish peroxidase (HRP)-strepavidin is added to the plate as the detection reagent. The amount of labelled reference antibody bound to the target antigen is detected following addition of a chromogenic substrate (e.g., TMB (3,3′,5,5′-tetramethylbenzidine) or ABTS (2,2″-azino-di-(3-ethylbenzthiazoline-6-sulfonate)), which are known in the art. Optical density readings (OD units) are made using a spectrometer (e.g. SpectraMax® M2 spectrometer (Molecular Devices)). The response (OD units) corresponding to zero percent inhibition is determined from wells without any competing antibody. The response (OD units) corresponding to 100% inhibition, i.e. the assay background, is determined from wells without any labelled reference antibody or test antibody. Percent inhibition of labelled reference antibody to FIX or FIXa by the test antibody (or the unlabelled reference antibody) at each concentration is calculated as follows: % inhibition=(1−(OD units−100% inhibition)/(0% inhibition−100% inhibition))*100.

The person skilled in the art will understand that similar assays may be performed to determine if two or more anti-FX/FXa antibodies shares a binding region, a bin and/or competitively binds the antigen. Persons skilled in the art will also appreciate that the competition assay can be performed using various detection systems known in the art.

A test antibody competes with the reference antibody for binding to the antigen if an excess of one antibody (e.g., 1, 5, 10, 20, 100, 1000, 10000 or 100000 fold) inhibits binding of the other antibody, e.g., by at least 50%, 75%, 90%, 95% or 99%, as measured in a competitive binding assay.

Unless otherwise indicated competition is determined using a competitive ELISA assay as described above.

The term “binding affinity” is herein used as a measure of the strength of a non-covalent interaction between two molecules, e.g. an antibody, or fragment thereof, and an antigen. The term “binding affinity” is used to describe monovalent interactions.

Binding affinity between two molecules, e.g. an antibody, or fragment thereof, and an antigen, through a monovalent interaction may be quantified by determining the equilibrium dissociation constant (K_{D}). K_{D} can be determined by measurement of the kinetics of complex formation and dissociation, e.g. by the Surface Plasmon Resonance (SPR) method or the Isothermal Titration calorimetry (ITC) method. The rate constants corresponding to the association and the dissociation of a monovalent complex are referred to as the association rate constant kₐ (or kₒₙ) and dissociation rate constant k_{d} (or k_{off}), respectively. K_{D} is related to kₐ and k_{d} through the equation K_{D}=k_{d}/kₐ.

Following the above definition, binding affinities associated with different molecular interactions, such as comparison of the binding affinity of different antibodies for a given antigen, may be compared by comparison of the K_{D} values for the individual antibody/antigen complexes.

The value of the dissociation constant can be determined directly by well-known methods. Standard assays to evaluate the binding ability of ligands such as antibodies towards targets are known in the art and include, for example, ELISAs, Western blots, RIAs, and flow cytometry analysis. The binding kinetics and binding affinity of the antibody also can be assessed by standard assays known in the art, such as SPR. Preferably, however, isothermal titration calorimetry (ITC) may be used to measure affinities for an antibody/target interaction as well as to derive thermodynamic parameters for the interaction.

A competitive binding assay can be conducted in which the binding of the antibody to the target is compared to the binding of the target by another ligand of that target, such as another antibody.

The K_{D} of an antibody of the invention for its target may be less than 100 μM such as less than 10 μM, such as less than 9 μM, such as less than 8 μM, such as less than 7 μM, such as less than 6 μM, such as less than 5 μM, such as less than 4 μM, such as less than 3 μM, such as less than 2 μM, such as less than 1 μM, such as less than 0.9 μM, such as less than 0.8 μM, such as less than 0.7 μM, such as less than 0.6 μM, such as less than 0.5 μM, such as less than 0.4 μM, such as less than 0.3 μM, such as less than 0.2 μM, such as less than 0.1 μM. In one such embodiment the antibody is a bispecific antibody comprising an anti-FX arm with a K_{D} towards FX of less than 100 μM such as less than 10 μM, such as less than 9 μM, such as less than 8 μM, such as less than 7 μM, such as less than 6 μM, such as less than 5 μM, such as less than 4 μM, such as less than 3 μM, such as less than 2 μM, such as less than 1 μM, such as less than 0.9 μM, such as less than 0.8 μM, such as less than 0.7 μM, such as less than 0.6 μM, such as less than 0.5 μM, such as less than 0.4 μM, such as less than 0.3 μM, such as less than 0.2 μM, such as less than 0.1 μM, such as less than 0.09 μM, such as less than 0.08 μM, such as less than 0.07 μM, such as less than 0.06 μM, such as less than 0.05 μM, such as less than 0.04 μM, such as less than 0.03 μM, such as less than 0.02 μM, such as less than 0.01 μM, such as less than 9 nM, such as less than 8 nM, such as less than 7 nM, such as less than 6 nM, such as less than 5 nM, such as less than 4 nM, such as less than 3 nM, such as less than 2 nM, such as less than 1 nM such as less than 0.5 nM.

The antibodies and antibody fragment thereof as described herein may be combined with other antibodies and antibody fragments known in the art creating bispecific, trispecific or multispecific antibody molecules. Compounds mimicking FVIII cofactor function have previously been created using other FIX(a) and FX(a) binding domains, which may potentially each substitute for the FIX(a) and/or FX(a) binding domains described herein. It is thus clear that the FIX(a) and FX(a) binding domains of the invention are of separate interest as individual component (intermediate) molecules, as part of a bi-, tri- or multispecific antibody comprising at least one FIX(a) and/or FX(a) binding domain.

The activity of procoagulant antibodies including bi-, tri and multispecific antibodies may be determined by methods known in the art. Standard assays include whole blood-Thrombin-Generation Test (TGT), measuring of clotting time by thrombelastography (TEG) and FXa generation assays.

Identity

The term “identity” as known in the art, refers to a relationship between the sequences of two or more polypeptides, as determined by comparing the sequences. In the art, “identity” also means the degree of sequence relatedness between polypeptides, as determined by the number of matches between strings of two or more amino acid residues. “Identity” measures the percent of identical matches between the smaller of two or more sequences with gap alignments (if any) addressed by a particular mathematical model or computer program (i.e., “algorithms”). Identity of related polypeptides can be readily calculated by known methods.

In the present invention similarity and identity were determined using Needleman (Needleman et al. J. Mol. Biol. 1970; 48:443-453) from EMBOSS-6.6.0 using the parameters 10 and 0.5 for gaps opening and extensions, respectively (gapopen=10, gapextend=0.5).

Pharmaceutical Formulations

In another aspect, the present invention provides compositions and formulations comprising compounds of the invention, such as the antibodies described herein. For example, the invention provides a pharmaceutical composition that comprises one or more antibodies of the invention, formulated together with a pharmaceutically acceptable carrier.

Accordingly, one object of the invention is to provide a pharmaceutical formulation comprising such an antibody which is present in a concentration from 0.25 mg/ml to 250 mg/ml, and wherein said formulation has a pH from 2.0 to 10.0. The formulation may further comprise one or more of a buffer system, a preservative, a tonicity agent, a chelating agent, a stabilizer, or a surfactant, as well as various combinations thereof. The use of preservatives, isotonic agents, chelating agents, stabilizers and surfactants in pharmaceutical compositions is well-known to the skilled person. Reference may be made to Remington: The Science and Practice of Pharmacy, 19th edition, 1995.

In one embodiment the pharmaceutical formulation is an aqueous formulation. Such a formulation is typically a solution or a suspension, but may also include colloids, dispersions, emulsions, and multi-phase materials. The term “aqueous formulation” is defined as a formulation comprising at least 50% w/w water. Likewise, the term “aqueous solution” is defined as a solution comprising at least 50% w/w water, and the term “aqueous suspension” is defined as a suspension comprising at least 50% w/w water.

In another embodiment the pharmaceutical formulation is a freeze-dried formulation, to which a solvent and/or a diluent is added prior to use.

In a further aspect, the pharmaceutical formulation comprises an aqueous solution of such an antibody, and a buffer, wherein the antibody is present in a concentration from 1 mg/ml or above, and wherein said formulation has a pH from about 2.0 to about 10.0.

In one embodiment the present invention relates to an injection device with content of said composition. In some embodiments the pharmaceutical composition of the invention is intended for use and/or contained in an injection device. In some embodiments, the injection device is a disposable, pre-filled, multi-dose pen of the FlexTouch® type (supplier Novo Nordisk A/S, Denmark). In some embodiments the injection device is a single shot device.

In some embodiments the injection device is a fixed dose device, such as one configured to deliver multiple predetermined doses of drug, sometimes referred to as a multiple fixed dose device or a fixed dose, multi-shot device.

In one embodiment the pharmaceutical composition of the invention is administered using an injection device comprising a tube having a needle gauge of 20 or greater.

In one embodiment a bispecific antibody according to table 1 herein is administered using a an injection device comprising a tube having a needle gauge of 20 or greater.

In one embodiment a bispecific antibody according to table 1 herein is administered using a an injection device comprising a tube having a needle gauge of 20 to 36. In one such embodiment the bispecific antibody is selected from a list consisting of bimAb05-0745, bimAb05-3761, bimAb05-3761, bimAb05-2112, bimAb05-2113, bimAb05-2114, bimAb05-3769, bimAb05-4271, bimAb05-4756, bimAb05-0396, bimAb05-0417 and bimAb05-0438,

Administration and Dosages

A compound of the invention, such as an antibody, may be administered parenterally, such as intravenously, such as intramuscularly, such as subcutaneously. Alternatively, an antibody of the invention may be administered via a non-parenteral route, such as periorally or topically. An antibody of the invention may be administered prophylactically. An antibody of the invention may be administered therapeutically (on demand).

The dose of the compounds to be delivered may be from about 0.01 mg to 500 mg of the compound per day, preferably from about 0.1 mg to 250 mg per day, and more preferably from about 0.5 mg to about 250 mg per day, per week, per second week or per month as loading and maintenance doses, depending on the severity of the condition. A suitable dose may also be adjusted for a particular compound based on the properties of that compound, including its in vivo half-life or mean residence time and its biological activity. For example, compounds to be delivered could in one embodiment be administered once weekly, or in another embodiment once every second week or in another embodiment once monthly and in either of said embodiments in a dose of for example 0.025, 0.05, 0.075, 0.1, 0.125, 0.15, 0.175, 0.2, 0.25, 0.3, 0.35, 0.4, 0.45, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5 or 10 mg per kg body weight.

The compositions containing the compounds as disclosed herein can be administered for prophylactic and/or in some embodiments therapeutic treatments. In therapeutic applications, compositions are administered to a subject already suffering from a disease, such as any bleeding disorder as described above, in an amount sufficient to cure, alleviate or partially arrest the disease and its complications. An amount adequate to accomplish this is defined as “therapeutically effective amount”. As will be understood by the person skilled in the art amounts effective for this purpose will depend on the severity of the disease or injury as well as the weight and general state of the subject.

### Embodiments

The invention is further described by the following embodiments:
1. An antibody or antigen-binding fragment thereof capable of binding to Factor IX (FIX) according to SEQ ID NO:1 and/or the activated form thereof (FIXa).
2. The antibody or antigen-binding fragment thereof according to embodiment 1, wherein the antibody or antigen-binding fragment thereof is part of “BinB” as described herein.
3. The antibody or antigen-binding fragment thereof according to embodiment 1, wherein the antibody or antigen-binding fragment thereof competes with a reference antibody wherein the reference antibody comprises
   a. a heavy chain variable domain identified by SEQ ID NO:35 and a light chain variable domain identified by SEQ ID NO:39,
   b. a heavy chain variable domain identified by SEQ ID NO:43 and a light chain variable domain identified by SEQ ID NO:47, or
   c. a heavy chain variable domain identified by SEQ ID NO:51 and a light chain variable domain identified by SEQ ID NO:55, or
   d. a heavy chain variable domain identified by SEQ ID NO:67 and a light chain variable domain identified by SEQ ID NO:71, or
   e. a heavy chain variable domain identified by SEQ ID NO:1202 and a light chain variable domain identified by SEQ ID NO:1206, or
   f. a heavy chain variable domain identified by SEQ ID NO:1210 and a light chain variable domain identified by SEQ ID NO:1214, or
   g. a heavy chain variable domain identified by SEQ ID NO:1218 and a light chain variable domain identified by SEQ ID NO:1222, or
   h. a heavy chain variable domain identified by SEQ ID NO:1226 and a light chain variable domain identified by SEQ ID NO:1230, or
   i. a heavy chain variable domain identified by SEQ ID NO:1234 and a light chain variable domain identified by SEQ ID NO:1238, or
   j. a heavy chain variable domain identified by SEQ ID NO:1242 and a light chain variable domain identified by SEQ ID NO:1246.
4. The antibody or antigen-binding fragment thereof according to the previous embodiment, wherein the reference antibody is a Fab.
5. The antibody or antigen-binding fragment thereof according to any of the previous embodiments, wherein the antibody or antigen-binding fragment thereof comprises
   a. a heavy chain variable domain at least 90% identical to the sequence identified by SEQ ID NO:35 and a light chain variable domain at least 90% identical to the sequence identified by SEQ ID NO:39; or
   b. a heavy chain variable domain at least 90% identical to the sequence identified by SEQ ID NO:43 and a light chain variable domain at least 90% identical to the sequence identified by SEQ ID NO:47; or
   c. a heavy chain variable domain at least 90% identical to the sequence identified by SEQ ID NO:51 and a light chain variable domain at least 90% identical to the sequence identified by SEQ ID NO:55; or
   d. a heavy chain variable domain at least 90% identical to the sequence identified by SEQ ID NO:67 and a light chain variable domain at least 90% identical to the sequence identified by SEQ ID NO:71, or
   e. a heavy chain variable domain at least 90% identical to the sequence identified by SEQ ID NO:1202 and a light chain variable domain at least 90% identical to the sequence identified by SEQ ID NO:1206, or
   f. a heavy chain variable domain at least 90% identical to the sequence identified by SEQ ID NO:1210 and a light chain variable domain at least 90% identical to the sequence identified by SEQ ID NO:1214, or
   g. a heavy chain variable domain at least 90% identical to the sequence identified by SEQ ID NO:1218 and a light chain variable domain at least 90% identical to the sequence identified by SEQ ID NO:1222, or
   h. a heavy chain variable domain at least 90% identical to the sequence identified by SEQ ID NO:1226 and a light chain variable domain at least 90% identical to the sequence identified by SEQ ID NO:1230, or
   i. a heavy chain variable domain at least 90% identical to the sequence identified by SEQ ID NO:1234 and a light chain variable domain at least 90% identical to the sequence identified by SEQ ID NO:1238, or
   j. a heavy chain variable domain at least 90% identical to the sequence identified by SEQ ID NO:1242 and a light chain variable domain at least 90% identical to the sequence identified by SEQ ID NO:1246.
6. The antibody or antigen-binding fragment thereof according to embodiment 5, wherein the heavy chain variable domain is at least 92, 94, 96, 97, 98, 99, 99.1 or 99.2% identical to the sequence identified by SEQ ID NO:35, 43, 51, 67, 1202, 1210, 1218, 1226, 1234 or 1242 respectively.
7. The antibody or antigen-binding fragment thereof according to embodiment 5, wherein the light chain variable domain is at least 92, 94, 96, 97, 98, 99, 99.1 or 99.2% identical to the sequence identified by SEQ ID NO: 39, 47, 55, 71, 1206, 1214, 1222, 1230, 1238 or 1246 respectively.
8. The antibody or antigen-binding fragment thereof according to embodiment 6 and 7, wherein both the heavy chain variable domain and the light chain variable domain are at least 92, 94, 96, 97, 98, 99, 99.1 or 99.2% identical to the identified SEQ IDs.
9. The antibody or antigen-binding fragment thereof according to any of the previous embodiments, wherein the antibody or antigen-binding fragment thereof is capable of binding an epitope comprising one or more of the amino acid residues L337, R338, S339, T340, K341 and T343 of SEQ ID NO:1.
10. The antibody or antigen-binding fragment thereof according to any of the previous embodiments, wherein the antibody or antigen-binding fragment thereof is capable of binding an epitope comprising amino acid residue R338 of SEQ ID NO:1.
11. The antibody or antigen-binding fragment thereof according to any of the previous embodiments, wherein the antibody or antigen-binding fragment thereof is capable of binding an epitope comprising amino acid residues R338 and K341 of SEQ ID NO:1.
12. The antibody or antigen-binding fragment thereof according to any of the previous embodiments, wherein the antibody or antigen-binding fragment thereof is capable of binding an epitope comprising two or three of the amino acid residues L337, R338, S339, T340, K341 and T343 of SEQ ID NO:1.
13. The antibody or antigen-binding fragment thereof according to any of the previous embodiments, wherein the antibody or antigen-binding fragment thereof is capable of binding an epitope comprising four or five of the amino acid residues L337, R338, S339, T340, K341 and T343 of SEQ ID NO:1.
14. The antibody or antigen-binding fragment thereof according to any of the previous embodiments, wherein the antibody or antigen-binding fragment thereof is capable of binding an epitope comprising
   a. R338, S339, T340, K341 and T343,
   b. L337, S339, T340, K341 and T343,
   c. L337, R338, T340, K341 and T343,
   d. L337, R338, S339, K341 and T343,
   e. L337, R338, S339, T340 and T343
   f. L337, R338, S339, T340 and K341,
   g. L337, R338, S339 and T340, or
   h. R338, T340 and K341
of SEQ ID NO:1.
15. The antibody or antigen-binding fragment thereof according to any of embodiments 1-11, wherein the antibody or antigen-binding fragment thereof is capable of binding an epitope comprising amino acid residues
   a. R338, T340 and K341 of SEQ ID NO:1, or
   b. L337, R338, S339, T340 and K341 of SEQ ID NO:1.
16. The antibody or antigen-binding fragment thereof according to any of the previous embodiments, comprising a paratope having the following amino acid residues
   a. H30, D31, W53, D56, S102, S104, Y106 and N107 in the heavy chain variable domain (SEQ ID NO:67) and Y91 and S92 in the light chain variable domain (SEQ ID NO:71), optionally comprising one, two or three amino acid substitutions in the ten recited paratope amino acid residues, or
   b. H30, D31, W53, S102, S104, Y106 and N107 in the heavy chain variable domain (SEQ ID NO:51) and residues Y91 and S92 in the light chain variable domain (SEQ ID NO:55), optionally comprising one, two or three amino acid substitutions in the nine recited paratope amino acid residues.
17. The antibody or antigen-binding fragment thereof according to any of embodiments 1-16, comprising a paratope having the following amino acid residues D30, D31, W53, S102, S104 and N107 in the heavy chain variable domain (SEQ ID NO:35) and Y91 and S92 in the light chain variable domain (SEQ ID NO:39), optionally comprising one, two or three amino acid substitutions in the eight recited paratope amino acid residues.
18. The antibody or antigen-binding fragment thereof according to embodiment 16 or 17 wherein said substitution(s) is/are a conservative substitution(s).
19. The antibody or antigen-binding fragment thereof according to any of the previous embodiments, wherein the antibody or antigen-binding fragment thereof comprises
   a.
      i. three heavy chain CDR sequences with at most 10 amino acid changes compared to the CDR sequences of the heavy chain variable domain identified by SEQ ID NO:35 and
      ii. three light chain CDR sequences with at most 10 amino acid changes compared to the CDR sequences of the light chain variable domain identified by SEQ ID NO:39, or
   b.
      i. three heavy chain CDR sequences with at most 10 amino acid changes compared to the CDR sequences of the heavy chain variable domain identified by SEQ ID NO:43 and
      ii. three light chain CDR sequences with at most 10 amino acid changes compared to the CDR sequences of the light chain variable domain identified by SEQ ID NO:47, or
   c.
      i. three heavy chain CDR sequences with at most 10 amino acid changes compared to the CDR sequences of the heavy chain variable domain identified by SEQ ID NO:51 and
      ii. three light chain CDR sequences with at most 10 amino acid changes compared to the CDR sequences of the light chain variable domain identified by SEQ ID NO:55, or
   d.
      i. three heavy chain CDR sequences with at most 10 amino acid changes compared to the CDR sequences of the heavy chain variable domain identified by SEQ ID NO:67 and
      ii. three light chain CDR sequences with at most 10 amino acid changes compared to the CDR sequences of the light chain variable domain identified by SEQ ID NO:71, or
   e.
      i. three heavy chain CDR sequences with at most 10 amino acid changes compared to the CDR sequences of the heavy chain variable domain identified by SEQ ID NO:1202 and
      ii. three light chain CDR sequences with at most 10 amino acid changes compared to the CDR sequences of the light chain variable domain identified by SEQ ID NO:1206, or
   f.
      i. three heavy chain CDR sequences with at most 10 amino acid changes compared to the CDR sequences of the heavy chain variable domain identified by SEQ ID NO:1210 and
      ii. three light chain CDR sequences with at most 10 amino acid changes compared to the CDR sequences of the light chain variable domain identified by SEQ ID NO:1214, or
   g.
      i. three heavy chain CDR sequences with at most 10 amino acid changes compared to the CDR sequences of the heavy chain variable domain identified by SEQ ID NO:1218 and
      ii. three light chain CDR sequences with at most 10 amino acid changes compared to the CDR sequences of the light chain variable domain identified by SEQ ID NO:1222, or
   h.
      i. three heavy chain CDR sequences with at most 10 amino acid changes compared to the CDR sequences of the heavy chain variable domain identified by SEQ ID NO:1226 and
      ii. three light chain CDR sequences with at most 10 amino acid changes compared to the CDR sequences of the light chain variable domain identified by SEQ ID NO:1230, or
   i.
      i. three heavy chain CDR sequences with at most 10 amino acid changes compared to the CDR sequences of the heavy chain variable domain identified by SEQ ID NO:1234 and
      ii. three light chain CDR sequences with at most 10 amino acid changes compared to the CDR sequences of the light chain variable domain identified by SEQ ID NO:1238, or
   j.
      i. three heavy chain CDR sequences with at most 10 amino acid changes compared to the CDR sequences of the heavy chain variable domain identified by SEQ ID NO:1242 and
      ii. three light chain CDR sequences with at most 10 amino acid changes compared to the CDR sequences of the light chain variable domain identified by SEQ ID NO:1246.
20. The antibody or antigen-binding fragment thereof according to embodiment 19, wherein the three heavy chain CDR sequences have at most 9, such as 8, such as 7 or such as 6 amino acid changes compared to the CDRs of the identified SEQ ID NOs.
21. The antibody or antigen-binding fragment thereof according to embodiment 19, wherein the three heavy chain CDR sequences have at most 5, such as 4, such as 3, such as 2 or at most 1 amino acid changes compared to the CDRs of the identified SEQ ID NOs.
22. The antibody or antigen-binding fragment thereof according to embodiment 19, wherein the three light chain CDR sequences have at most 9, such as 8, such as 7 or such as 6 amino acid changes compared to the CDRs of the identified SEQ ID NOs.
23. The antibody or antigen-binding fragment thereof according to embodiment 19, wherein the three light chain CDR sequences have at most 5, such as 4, such as 3, such as 2 or at most 1 amino acid changes compared to the CDRs of the identified SEQ IDs.
24. The antibody or antigen-binding fragment thereof according to any of the previous embodiments, wherein the antibody or antigen-binding fragment thereof comprises
   a. the CDR sequences of the heavy chain variable domain identified by SEQ ID NO:35 and the CDR sequences of the light chain variable domain identified by SEQ ID NO:39, or
   b. the CDR sequences of the heavy chain variable domain identified by SEQ ID NO:43 and the CDR sequences of the light chain variable domain identified by SEQ ID NO:47, or
   c. the CDR sequences of the heavy chain variable domain identified by SEQ ID NO:51 and the CDR sequences of the light chain variable domain identified by SEQ ID NO:55, or
   d. the CDR sequences of the heavy chain variable domain identified by SEQ ID NO:67 and the CDR sequences of the light chain variable domain identified by SEQ ID NO:71, or
   e. the CDR sequences of the heavy chain variable domain identified by SEQ ID NO:1202 and the CDR sequences of the light chain variable domain identified by SEQ ID NO:1206, or
   f. the CDR sequences of the heavy chain variable domain identified by SEQ ID NO:1210 and the CDR sequences of the light chain variable domain identified by SEQ ID NO:1214, or
   g. the CDR sequences of the heavy chain variable domain identified by SEQ ID NO:1218 and the CDR sequences of the light chain variable domain identified by SEQ ID NO:1222, or
   h. the CDR sequences of the heavy chain variable domain identified by SEQ ID NO:1226 and the CDR sequences of the light chain variable domain identified by SEQ ID NO:1230, or
   i. the CDR sequences of the heavy chain variable domain identified by SEQ ID NO:1234 and the CDR sequences of the light chain variable domain identified by SEQ ID NO:1238, or
   j. the CDR sequences of the heavy chain variable domain identified by SEQ ID NO:1242 and the CDR sequences of the light chain variable domain identified by SEQ ID NO:1246.
25. The antibody or antigen-binding fragment thereof according to any of the previous embodiments, wherein the antibody or antigen-binding fragment thereof comprises
   a. a heavy chain variable domain identified by SEQ ID NO:35 and a light chain variable domain identified by SEQ ID NO:39, or
   b. a heavy chain variable domain identified by SEQ ID NO:43 and a light chain variable domain identified by SEQ ID NO:47, or
   c. a heavy chain variable domain identified by SEQ ID NO:51 and a light chain variable domain identified by SEQ ID NO:55, or
   d. a heavy chain variable domain identified by SEQ ID NO:67 and a light chain variable domain identified by SEQ ID NO:71, or
   e. a heavy chain variable domain identified by SEQ ID NO:1202 and a light chain variable domain identified by SEQ ID NO:1206, or
   f. a heavy chain variable domain identified by SEQ ID NO:1210 and a light chain variable domain identified by SEQ ID NO:1214, or
   g. a heavy chain variable domain identified by SEQ ID NO:1218 and a light chain variable domain identified by SEQ ID NO:1222, or
   h. a heavy chain variable domain identified by SEQ ID NO:1226 and a light chain variable domain identified by SEQ ID NO:1230, or
   i. a heavy chain variable domain identified by SEQ ID NO:1234 and a light chain variable domain identified by SEQ ID NO:1238, or
   j. a heavy chain variable domain identified by SEQ ID NO:1242 and a light chain variable domain identified by SEQ ID NO:1246.
26. An antibody or antigen-binding fragment thereof capable of binding to FX (SEQ ID NO:2) and/or the activated form thereof (FXa).
27. The antibody or antigen-binding fragment thereof according to embodiment 26, wherein the antibody or antigen-binding fragment thereof is part of “Bin2”.
28. The antibody or antigen-binding fragment thereof according to embodiment 26, wherein the antibody or antigen-binding fragment thereof competes with a reference antibody wherein the reference antibody comprises
   a. a heavy chain variable domain identified by SEQ ID NO:467 and a light chain variable domain identified by SEQ ID NO:471, or
   b. a heavy chain variable domain identified by SEQ ID NO:483 and a light chain variable domain identified by SEQ ID NO:487, or
   c. a heavy chain variable domain identified by SEQ ID NO:707 and a light chain variable domain identified by SEQ ID NO:711, or
   d. a heavy chain variable domain identified by SEQ ID NO:731 and a light chain variable domain identified by SEQ ID NO:735, or
   e. a heavy chain variable domain identified by SEQ ID NO:907 and a light chain variable domain identified by SEQ ID NO:911, or
   f. a heavy chain variable domain identified by SEQ ID NO:1075 and a light chain variable domain identified by SEQ ID NO:1079.
29. The antibody according to any of the previous embodiments, wherein the reference antibody is a Fab.
30. The antibody or antigen-binding fragment thereof according to any of the previous embodiments, wherein the antibody or antigen-binding fragment thereof comprises a heavy chain variable domain at least 90% identical to the sequence identified by SEQ ID NO:467, 483, 707, 731, 907 or 1075 and a light chain variable domain at least 90% identical to the sequence identified by SEQ ID NO:471, 487, 711, 735, 911 or 1079.
31. The antibody or antigen-binding fragment thereof according to embodiment 30, wherein the heavy chain variable domain is at least 92, 94, 96, 97, 98, 99, 99.1 or 99.2% identical to the sequence identified by SEQ ID NO: 467, 483, 707, 731, 907 or 1075.
32. The antibody or antigen-binding fragment thereof according to embodiment 30, wherein the light chain variable domain is at least 92, 94, 96, 97, 98, 99, 99.1 or 99.2% identical to the sequence identified by SEQ ID NO:471, 487, 711, 735, 911 or 1079.
33. The antibody or antigen-binding fragment thereof according to embodiment 31 and 32, wherein both the heavy chain variable domain and the light chain variable domain are at least 92, 94, 96, 97, 98, 99 or 99.1% identical to the identified SEQ IDs.
34. The antibody or antigen-binding fragment thereof according to any of the previous embodiments, wherein the antibody or antigen-binding fragment thereof is capable of binding an epitope comprising one or more of the amino acid residues E103, Q104, V108, R113, T116, L117, D119, I125, T127, E228, F229, Y230, E266, R287, P291, I292, P304, L419, K420, D423, R424, M426, K427 and T428 of FX(a).
35. The antibody or antigen-binding fragment thereof according to any of the previous embodiments, wherein the antibody or antigen-binding fragment thereof is capable of binding an epitope comprising 15, 16, 17, 18, 19, 20, 21, 22, 23 or 24 of the amino acid residues E103, Q104, V108, R113, T116, L117, D119, I125, T127, E228, F229, Y230, E266, R287, P291, I292, P304, L419, K420, D423, R424, M426, K427 and T428 of FX(a)
36. The antibody or antigen-binding fragment thereof according to any of the previous embodiments, wherein the antibody or antigen-binding fragment thereof is capable of binding an epitope comprising the amino acid residues Y230, D423, R424 and K427 of FX(a).
37. The antibody or antigen-binding fragment thereof according to any of the previous embodiments, wherein the antibody or antigen-binding fragment thereof is capable of binding an epitope comprising the amino acid residues E103, Q104, V108, R113, T116, L117, D119, I125, T127, E228, F229, Y230, E266, R287, P291, I292, P304, L419, K420, D423, R424, M426, K427 and T428 of FX(a).
38. The antibody or antigen-binding fragment thereof according to any of the previous embodiments, comprising a paratope comprising the following amino acid residues K23, S25, G26, Y27, F29, W33, D52, S54, D55, F57, S77, H100, Y101, Y102, N103, S104 in the heavy chain variable domain (SEQ ID NO:467) and residues V29, S30, S31, Y33, Y50, Q52, S54, R55, R57 and D94 in the light chain variable domain (SEQ ID NO:471), optionally comprising one, two, three, four or five amino acid substitutions, deletions or insertions in the 26 recited paratope amino acid residues.
39. The antibody or antigen-binding fragment thereof according to any of the previous embodiments, wherein the antibody or antigen-binding fragment thereof is capable of binding an epitope comprising one or more of the amino acid residues E103, Q104, V108, R113, T116, L117, A118, D119, I125, T127, S227, E228, Y230, R287, I292, L303, P304, L419, K420, D423, R424, M426, K427 and T428 of FX(a).
40. The antibody or antigen-binding fragment thereof according to any of the previous embodiments, wherein the antibody or antigen-binding fragment thereof is capable of binding an epitope comprising 15, 16, 17, 18, 19, 20, 21, 22, 23 or 24 of the amino acid residues E103, Q104, V108, R113, T116, L117, A118, D119, I125, T127, S227, E228, Y230, R287, I292, L303, P304, L419, K420, D423, R424, M426, K427 and T428 of FX(a).
41. The antibody or antigen-binding fragment thereof according to any of the previous embodiments, wherein the antibody or antigen-binding fragment thereof is capable of binding an epitope comprising the amino acid residues E103, Q104, V108, R113, T116, L117, D119, I125, T127, E228, F229, Y230, E266, R287, P291, I292, P304, L419, K420, D423, R424, M426, K427 and T428 of FX(a).
42. The antibody or antigen-binding fragment thereof according to any of the previous embodiments, comprising a paratope comprising the following amino acid residues K23, G24, S25, G26, Y27, W33, D52, S54, D55, Y57, S77, L99, H100, Y101, Y102, N103 and S104 in the variable heavy chain domain (SEQ ID NO:483) and residues S30, S31, Y33, Y50, Q52, S54, R55, R57, Y92 and D94 in the light chain variable domain (SEQ ID NO:487), optionally comprising one, two, three, four or five amino acid substitutions, deletions or insertions in the 27 recited paratope amino acid residues.
43. The antibody or antigen-binding fragment thereof according to embodiment 38 or 42 wherein said substitution(s) is/are a conservative substitution(s).
44. The antibody or antigen-binding fragment thereof according to any of embodiments 26-43, wherein the antibody or antigen-binding fragment thereof comprises
   a. three heavy chain CDR sequences with at most 10 amino acid changes compared to the CDR sequences of the heavy chain variable domain identified by SEQ ID NO:467 and
      three light chain CDR sequences with at most 10 amino acid changes compared to the CDR sequences of the light chain variable domain identified by SEQ ID NO:471; or
   b. three heavy chain CDR sequences with at most 10 amino acid changes compared to the CDR sequences of the heavy chain variable domain identified by SEQ ID NO:483, and
      three light chain CDR sequences with at most 10 amino acid changes compared to the CDR sequences of the light chain variable domain identified by SEQ ID NO:487.
45. The antibody or antigen-binding fragment thereof according to embodiment 44, wherein the three heavy chain CDR sequences have at most 9, such as 8, such as 7 or such as 6 amino acid changes compared to the CDRs of the identified SEQ IDs.
46. The antibody or antigen-binding fragment thereof according to embodiment 44, wherein the three heavy chain CDR sequences have at most 5, such as 4, such as 3, such as 2 or at most 1 amino acid changes compared to the CDRs of the identified SEQ IDs.
47. The antibody or antigen-binding fragment thereof according to embodiment 44, wherein the three light chain CDR sequences have at most 9, such as 8, such as 7 or such as 6 amino acid changes compared to the CDRs of the identified SEQ IDs.
48. The antibody or antigen-binding fragment thereof according to embodiment 44, wherein the three light chain CDR sequences have at most 5, such as 4, such as 3, such as 2 or at most 1 amino acid changes compared to the CDRs of the identified SEQ IDs.
49. The antibody or antigen-binding fragment thereof according to any of the previous embodiments, wherein the antibody or antigen-binding fragment thereof comprises the CDR sequences of
   a. the heavy chain variable domain identified by SEQ ID NO:467 and the CDR sequences of the light chain variable domain identified by SEQ ID NO:471; or
   b. the heavy chain variable domain identified by SEQ ID NO:483 and the CDR sequences of the light chain variable domain identified by SEQ ID NO:487; or
   c. the heavy chain variable domain identified by SEQ ID NO:555 and the CDR sequences of the light chain variable domain identified by SEQ ID NO:559; or
   d. the heavy chain variable domain identified by SEQ ID NO:587 and the CDR sequences of the light chain variable domain identified by SEQ ID NO:591; or
   e. the heavy chain variable domain identified by SEQ ID NO:707 and the CDR sequences of the light chain variable domain identified by SEQ ID NO:711; or
   f. the heavy chain variable domain identified by SEQ ID NO:731 and the CDR sequences of the light chain variable domain identified by SEQ ID NO:735; or
   g. the heavy chain variable domain identified by SEQ ID NO:907 and the CDR sequences of the light chain variable domain identified by SEQ ID NO:911; or
   h. the heavy chain variable domain identified by SEQ ID NO:1075 and the CDR sequences of the light chain variable domain identified by SEQ ID NO:1079.
50. The antibody or antigen-binding fragment thereof according to any of the previous embodiments, wherein the antibody or antigen-binding fragment thereof comprises
   a. a heavy chain variable domain identified by SEQ ID NO:467 and a light chain variable domain identified by SEQ ID NO:471; or
   b. a heavy chain variable domain identified by SEQ ID NO:483 and a light chain variable domain identified by SEQ ID NO:487; or
   c. a heavy chain variable domain identified by SEQ ID NO:555 and a light chain variable domain identified by SEQ ID NO:559; or
   d. a heavy chain variable domain identified by SEQ ID NO:587 and a light chain variable domain identified by SEQ ID NO:591; or
   e. a heavy chain variable domain identified by SEQ ID NO:707 and a light chain variable domain identified by SEQ ID NO:711; or
   f. a heavy chain variable domain identified by SEQ ID NO:731 and a light chain variable domain identified by SEQ ID NO:735; or
   g. a heavy chain variable domain identified by SEQ ID NO:907 and a light chain variable domain identified by SEQ ID NO:911; or
   h. a heavy chain variable domain identified by SEQ ID NO:1075 and a light chain variable domain identified by SEQ ID NO:1079.
51. A multispecific antibody or antigen-binding fragment thereof capable of binding to FIX according to SEQ ID NO:1 or the activated form thereof (FIXa), and FX (SEQ ID NO:2) or the activated form thereof (FXa).
52. The multispecific antibody or antigen-binding fragment thereof according to embodiment 51, wherein the antibody comprises an antibody or antigen-binding fragment thereof according to any of the previous embodiments 1-50.
53. The multispecific antibody or antigen-binding fragment thereof according to embodiment 51, wherein the antibody comprises an antibody or antigen-binding fragment thereof according to any of the previous embodiments 2-25.
54. The multispecific antibody or antigen-binding fragment thereof according to embodiment 51, wherein the antibody comprises an antibody or antigen-binding fragment thereof according to any of the previous embodiments 26-50.
55. The multispecific antibody or antigen-binding fragment thereof according to embodiment 51, wherein the antibody comprises an antibody or antigen-binding fragment thereof according to any of the previous embodiments 27-50.
56. The multispecific antibody or antigen-binding fragment thereof according to embodiment 51, wherein the antibody comprises an antibody or antigen-binding fragment thereof according to any of the previous embodiments 1-25 and an antigen-binding fragment according to any of the previous embodiments 26-50.
57. The multispecific antibody or antigen-binding fragment thereof according to embodiment 51, wherein the antibody comprises an antibody or antigen-binding fragment thereof according to any of the previous embodiments 2-25 and an antibody or antigen-binding fragment thereof according to any of the previous embodiments 27-50.
58. The multispecific antibody or antigen-binding fragment thereof according to any of embodiments 51-57 comprising
   a. the anti-FIX(a) antibody heavy chain CDR3 sequence identified by SEQ ID NO:38, optionally comprising 1, 2 or 3 amino acid substitutions and/or deletions and/or insertions; and
      the anti-FIX(a) antibody light chain CDR3 sequence identified by SEQ ID NO:42, optionally comprising 1, 2 or 3 amino acid substitutions and/or deletions and/or insertions; and
      the anti-FX(a) antibody heavy chain CDR3 sequence identified by SEQ ID NO:470, optionally comprising 1, 2 or 3 amino acid substitutions and/or deletions and/or insertions; and
      the anti-FX(a) antibody light chain CDR3 sequence identified by SEQ ID NO:474, optionally comprising 1, 2 or 3 amino acid substitutions and/or deletions and/or insertions; or
   b. the anti-FIX(a) antibody heavy chain CDR3 sequence identified by SEQ ID NO:38, optionally comprising 1, 2 or 3 amino acid substitutions and/or deletions and/or insertions; and
      the anti-FIX(a) antibody light chain CDR3 sequence identified by SEQ ID NO:42, optionally comprising 1, 2 or 3 amino acid substitutions and/or deletions and/or insertions; and
      the anti-FX(a) antibody heavy chain CDR3 sequence identified by SEQ ID NO: 486, optionally comprising 1, 2 or 3 amino acid substitutions and/or deletions and/or insertions; and
      the anti-FX(a) antibody light chain CDR3 sequence identified by SEQ ID NO: 490, optionally comprising 1, 2 or 3 amino acid substitutions and/or deletions and/or insertions; or
   c. the anti-FIX(a) antibody heavy chain CDR3 sequence identified by SEQ ID NO:46, optionally comprising 1, 2 or 3 amino acid substitutions and/or deletions and/or insertions; and
      the anti-FIX(a) antibody light chain CDR3 sequence identified by SEQ ID NO:50, optionally comprising 1, 2 or 3 amino acid substitutions and/or deletions and/or insertions; and
      the anti-FX(a) antibody heavy chain CDR3 sequence identified by SEQ ID NO:470, optionally comprising 1, 2 or 3 amino acid substitutions and/or deletions and/or insertions; and
      the anti-FX(a) antibody light chain CDR3 sequence identified by SEQ ID NO:474, optionally comprising 1, 2 or 3 amino acid substitutions and/or deletions and/or insertions; or
   d. the anti-FIX(a) antibody heavy chain CDR3 sequence identified by SEQ ID NO:54, optionally comprising 1, 2 or 3 amino acid substitutions and/or deletions and/or insertions; and
      the anti-FIX(a) antibody light chain CDR3 sequence identified by SEQ ID NO:58, optionally comprising 1, 2 or 3 amino acid substitutions and/or deletions and/or insertions; and
      the anti-FX(a) antibody heavy chain CDR3 sequence identified by SEQ ID NO:470, optionally comprising 1, 2 or 3 amino acid substitutions and/or deletions and/or insertions; and
      the anti-FX(a) antibody light chain CDR3 sequence identified by SEQ ID NO:474, optionally comprising 1, 2 or 3 amino acid substitutions and/or deletions and/or insertions; or
   e. the anti-FIX(a) antibody heavy chain CDR3 sequence identified by SEQ ID NO:54, optionally comprising 1, 2 or 3 amino acid substitutions and/or deletions and/or insertions; and
      the anti-FIX(a) antibody light chain CDR3 sequence identified by SEQ ID NO:58, optionally comprising 1, 2 or 3 amino acid substitutions and/or deletions and/or insertions; and
      the anti-FX(a) antibody heavy chain CDR3 sequence identified by SEQ ID NO:486, optionally comprising 1, 2 or 3 amino acid substitutions and/or deletions and/or insertions; and
      the anti-FX(a) antibody light chain CDR3 sequence identified by SEQ ID NO:490, optionally comprising 1, 2 or 3 amino acid substitutions and/or deletions and/or insertions; or
   f. the anti-FIX(a) antibody heavy chain CDR3 sequence identified by SEQ ID NO:54, optionally comprising 1, 2 or 3 amino acid substitutions and/or deletions and/or insertions; and
      the anti-FIX(a) antibody light chain CDR3 sequence identified by SEQ ID NO:58, optionally comprising 1, 2 or 3 amino acid substitutions and/or deletions and/or insertions; and
      the anti-FX(a) antibody heavy chain CDR3 sequence identified by SEQ ID NO:558, optionally comprising 1, 2 or 3 amino acid substitutions and/or deletions and/or insertions; and
      the anti-FX(a) antibody light chain CDR3 sequence identified by SEQ ID NO:562, optionally comprising 1, 2 or 3 amino acid substitutions and/or deletions and/or insertions; or
   g. the anti-FIX(a) antibody heavy chain CDR3 sequence identified by SEQ ID NO:54, optionally comprising 1, 2 or 3 amino acid substitutions and/or deletions and/or insertions; and
      the anti-FIX(a) antibody light chain CDR3 sequence identified by SEQ ID NO:58, optionally comprising 1, 2 or 3 amino acid substitutions and/or deletions and/or insertions; and
      the anti-FX(a) antibody heavy chain CDR3 sequence identified by SEQ ID NO:590, optionally comprising 1, 2 or 3 amino acid substitutions and/or deletions and/or insertions; and
      the anti-FX(a) antibody light chain CDR3 sequence identified by SEQ ID NO:594, optionally comprising 1, 2 or 3 amino acid substitutions and/or deletions and/or insertions; or
   h. the anti-FIX(a) antibody heavy chain CDR3 sequence identified by SEQ ID NO:54, optionally comprising 1, 2 or 3 amino acid substitutions and/or deletions and/or insertions; and
      the anti-FIX(a) antibody light chain CDR3 sequence identified by SEQ ID NO:58, optionally comprising 1, 2 or 3 amino acid substitutions and/or deletions and/or insertions; and
      the anti-FX(a) antibody heavy chain CDR3 sequence identified by SEQ ID NO:710, optionally comprising 1, 2 or 3 amino acid substitutions and/or deletions and/or insertions; and
      the anti-FX(a) antibody light chain CDR3 sequence identified by SEQ ID NO:714, optionally comprising 1, 2 or 3 amino acid substitutions and/or deletions and/or insertions; or
   i. the anti-FIX(a) antibody heavy chain CDR3 sequence identified by SEQ ID NO:54, optionally comprising 1, 2 or 3 amino acid substitutions and/or deletions and/or insertions; and
      the anti-FIX(a) antibody light chain CDR3 sequence identified by SEQ ID NO:58, optionally comprising 1, 2 or 3 amino acid substitutions and/or deletions and/or insertions; and
      the anti-FX(a) antibody heavy chain CDR3 sequence identified by SEQ ID NO:734, optionally comprising 1, 2 or 3 amino acid substitutions and/or deletions and/or insertions; and
      the anti-FX(a) antibody light chain CDR3 sequence identified by SEQ ID NO:738, optionally comprising 1, 2 or 3 amino acid substitutions and/or deletions and/or insertions; or
   j. the anti-FIX(a) antibody heavy chain CDR3 sequence identified by SEQ ID NO:54, optionally comprising 1, 2 or 3 amino acid substitutions and/or deletions and/or insertions; and
      the anti-FIX(a) antibody light chain CDR3 sequence identified by SEQ ID NO:58, optionally comprising 1, 2 or 3 amino acid substitutions and/or deletions and/or insertions; and
      the anti-FX(a) antibody heavy chain CDR3 sequence identified by SEQ ID NO:910, optionally comprising 1, 2 or 3 amino acid substitutions and/or deletions and/or insertions; and
      the anti-FX(a) antibody light chain CDR3 sequence identified by SEQ ID NO:914, optionally comprising 1, 2 or 3 amino acid substitutions and/or deletions and/or insertions; or
   k. the anti-FIX(a) antibody heavy chain CDR3 sequence identified by SEQ ID NO:54, optionally comprising 1, 2 or 3 amino acid substitutions and/or deletions and/or insertions; and
      the anti-FIX(a) antibody light chain CDR3 sequence identified by SEQ ID NO:58, optionally comprising 1, 2 or 3 amino acid substitutions and/or deletions and/or insertions; and
      the anti-FX(a) antibody heavy chain CDR3 sequence identified by SEQ ID NO:1078, optionally comprising 1, 2 or 3 amino acid substitutions and/or deletions and/or insertions; and
      the anti-FX(a) antibody light chain CDR3 sequence identified by SEQ ID NO:1082, optionally comprising 1, 2 or 3 amino acid substitutions and/or deletions and/or insertions; or
   l. the anti-FIX(a) antibody heavy chain CDR3 sequence identified by SEQ ID NO:70, optionally comprising 1, 2 or 3 amino acid substitutions and/or deletions and/or insertions; and
      the anti-FIX(a) antibody light chain CDR3 sequence identified by SEQ ID NO:74, optionally comprising 1, 2 or 3 amino acid substitutions and/or deletions and/or insertions; and
      the anti-FX(a) antibody heavy chain CDR3 sequence identified by SEQ ID NO:470, optionally comprising 1, 2 or 3 amino acid substitutions and/or deletions and/or insertions; and
      the anti-FX(a) antibody light chain CDR3 sequence identified by SEQ ID NO:474, optionally comprising 1, 2 or 3 amino acid substitutions and/or deletions and/or insertions; or
   m. the anti-FIX(a) antibody heavy chain CDR3 sequence identified by SEQ ID NO:70, optionally comprising 1, 2 or 3 amino acid substitutions and/or deletions and/or insertions; and
      the anti-FIX(a) antibody light chain CDR3 sequence identified by SEQ ID NO:74, optionally comprising 1, 2 or 3 amino acid substitutions and/or deletions and/or insertions; and
      the anti-FX(a) antibody heavy chain CDR3 sequence identified by SEQ ID NO:486, optionally comprising 1, 2 or 3 amino acid substitutions and/or deletions and/or insertions; and
      the anti-FX(a) antibody light chain CDR3 sequence identified by SEQ ID NO:490, optionally comprising 1, 2 or 3 amino acid substitutions and/or deletions and/or insertions; or
   n. the anti-FIX(a) antibody heavy chain CDR3 sequence identified by SEQ ID NO:1205, optionally comprising 1, 2 or 3 amino acid substitutions and/or deletions and/or insertions; and
      the anti-FIX(a) antibody light chain CDR3 sequence identified by SEQ ID NO:1209, optionally comprising 1, 2 or 3 amino acid substitutions and/or deletions and/or insertions; and
      the anti-FX(a) antibody heavy chain CDR3 sequence identified by SEQ ID NO:1213, optionally comprising 1, 2 or 3 amino acid substitutions and/or deletions and/or insertions; and
      the anti-FX(a) antibody light chain CDR3 sequence identified by SEQ ID NO:1217, optionally comprising 1, 2 or 3 amino acid substitutions and/or deletions and/or insertions; or
   o. the anti-FIX(a) antibody heavy chain CDR3 sequence identified by SEQ ID NO:1221, optionally comprising 1, 2 or 3 amino acid substitutions and/or deletions and/or insertions; and
      the anti-FIX(a) antibody light chain CDR3 sequence identified by SEQ ID NO:1225, optionally comprising 1, 2 or 3 amino acid substitutions and/or deletions and/or insertions; and
      the anti-FX(a) antibody heavy chain CDR3 sequence identified by SEQ ID NO:470, optionally comprising 1, 2 or 3 amino acid substitutions and/or deletions and/or insertions; and
      the anti-FX(a) antibody light chain CDR3 sequence identified by SEQ ID NO:474, optionally comprising 1, 2 or 3 amino acid substitutions and/or deletions and/or insertions; or
   p. the anti-FIX(a) antibody heavy chain CDR3 sequence identified by SEQ ID NO:1229, optionally comprising 1, 2 or 3 amino acid substitutions and/or deletions and/or insertions; and
      the anti-FIX(a) antibody light chain CDR3 sequence identified by SEQ ID NO:1233, optionally comprising 1, 2 or 3 amino acid substitutions and/or deletions and/or insertions; and
      the anti-FX(a) antibody heavy chain CDR3 sequence identified by SEQ ID NO:470, optionally comprising 1, 2 or 3 amino acid substitutions and/or deletions and/or insertions; and
      the anti-FX(a) antibody light chain CDR3 sequence identified by SEQ ID NO:474, optionally comprising 1, 2 or 3 amino acid substitutions and/or deletions and/or insertions; or
   q. the anti-FIX(a) antibody heavy chain CDR3 sequence identified by SEQ ID NO:1237, optionally comprising 1, 2 or 3 amino acid substitutions and/or deletions and/or insertions; and
      the anti-FIX(a) antibody light chain CDR3 sequence identified by SEQ ID NO:1241, optionally comprising 1, 2 or 3 amino acid substitutions and/or deletions and/or insertions; and
      the anti-FX(a) antibody heavy chain CDR3 sequence identified by SEQ ID NO:470, optionally comprising 1, 2 or 3 amino acid substitutions and/or deletions and/or insertions; and
      the anti-FX(a) antibody light chain CDR3 sequence identified by SEQ ID NO:474, optionally comprising 1, 2 or 3 amino acid substitutions and/or deletions and/or insertions; or
   r. the anti-FIX(a) antibody heavy chain CDR3 sequence identified by SEQ ID NO:1245, optionally comprising 1, 2 or 3 amino acid substitutions and/or deletions and/or insertions; and
      the anti-FIX(a) antibody light chain CDR3 sequence identified by SEQ ID NO:1249, optionally comprising 1, 2 or 3 amino acid substitutions and/or deletions and/or insertions; and
      the anti-FX(a) antibody heavy chain CDR3 sequence identified by SEQ ID NO:470, optionally comprising 1, 2 or 3 amino acid substitutions and/or deletions and/or insertions; and
      the anti-FX(a) antibody light chain CDR3 sequence identified by SEQ ID NO:474, optionally comprising 1, 2 or 3 amino acid substitutions and/or deletions and/or insertions; or
59. The multispecific antibody or antigen-binding fragment thereof according to any of embodiments 51-57 comprising
   a. the anti-FIX(a) antibody heavy chain CDR1-3 sequences identified by SEQ ID NOs:36, 37 and 38, respectively, optionally comprising 1, 2 or 3 amino acid substitutions and/or deletions and/or insertions; and
      the anti-FIX(a) antibody light chain CDR1-3 sequences identified by SEQ ID NOs:40, 41 and 42, respectively, optionally comprising 1, 2 or 3 amino acid substitutions and/or deletions and/or insertions; and
      the anti-FX(a) antibody heavy chain CDR1-3 sequences identified by SEQ ID NOs:468, 469 and 470, respectively, optionally comprising 1, 2 or 3 amino acid substitutions and/or deletions and/or insertions; and
      the anti-FX(a) antibody light chain CDR1-3 sequences identified by SEQ ID NOs:472, 473 and 474, respectively, optionally comprising 1, 2 or 3 amino acid substitutions and/or deletions and/or insertions; or
   b. the anti-FIX(a) antibody heavy chain CDR1-3 sequences identified by SEQ ID NOs:36, 37 and 38, respectively, optionally comprising 1, 2 or 3 amino acid substitutions and/or deletions and/or insertions; and
      the anti-FIX(a) antibody light chain CDR1-3 sequences identified by SEQ ID NOs:40, 41 and 42, respectively, optionally comprising 1, 2 or 3 amino acid substitutions and/or deletions and/or insertions; and
      the anti-FX(a) antibody heavy chain CDR1-3 sequences identified by SEQ ID NOs:484, 485 and 486, respectively, optionally comprising 1, 2 or 3 amino acid substitutions and/or deletions and/or insertions; and
      the anti-FX(a) antibody light chain CDR1-3 sequences identified by SEQ ID NOs:488, 489 and 490, respectively, optionally comprising 1, 2 or 3 amino acid substitutions and/or deletions and/or insertions; or
   c. the anti-FIX(a) antibody heavy chain CDR1-3 sequences identified by SEQ ID NOs:44, 45 and 46, respectively, optionally comprising 1, 2 or 3 amino acid substitutions and/or deletions and/or insertions; and
      the anti-FIX(a) antibody light chain CDR1-3 sequences identified by SEQ ID NOs:48, 49 and 50, respectively, optionally comprising 1, 2 or 3 amino acid substitutions and/or deletions and/or insertions; and
      the anti-FX(a) antibody heavy chain CDR1-3 sequences identified by SEQ ID NOs:468, 469 and 470, respectively, optionally comprising 1, 2 or 3 amino acid substitutions and/or deletions and/or insertions; and
      the anti-FX(a) antibody light chain CDR1-3 sequences identified by SEQ ID NOs:472, 473 and 474, respectively, optionally comprising 1, 2 or 3 amino acid substitutions and/or deletions and/or insertions; or
   d. the anti-FIX(a) antibody heavy chain CDR1-3 sequences identified by SEQ ID NOs:52, 53 and 54, respectively, optionally comprising 1, 2 or 3 amino acid substitutions and/or deletions and/or insertions; and
      the anti-FIX(a) antibody light chain CDR1-3 sequences identified by SEQ ID NOs:56, 57 and 58, respectively, optionally comprising 1, 2 or 3 amino acid substitutions and/or deletions and/or insertions; and
      the anti-FX(a) antibody heavy chain CDR1-3 sequences identified by SEQ ID NOs:468, 469 and 470, respectively, optionally comprising 1, 2 or 3 amino acid substitutions and/or deletions and/or insertions; and
      the anti-FX(a) antibody light chain CDR1-3 sequences identified by SEQ ID NOs:472, 473 and 474, respectively, optionally comprising 1, 2 or 3 amino acid substitutions and/or deletions and/or insertions; or
   e. the anti-FIX(a) antibody heavy chain CDR1-3 sequences identified by SEQ ID NOs:52, 53 and 54, respectively, optionally comprising 1, 2 or 3 amino acid substitutions and/or deletions and/or insertions; and
      the anti-FIX(a) antibody light chain CDR1-3 sequences identified by SEQ ID NOs:56, 57 and 58, respectively, optionally comprising 1, 2 or 3 amino acid substitutions and/or deletions and/or insertions; and
      the anti-FX(a) antibody heavy chain CDR1-3 sequences identified by SEQ ID NOs:708, 709 and 710, respectively, optionally comprising 1, 2 or 3 amino acid substitutions and/or deletions and/or insertions; and
      the anti-FX(a) antibody light chain CDR1-3 sequences identified by SEQ ID NOs:712, 713 and 714, respectively, optionally comprising 1, 2 or 3 amino acid substitutions and/or deletions and/or insertions; or
   f. the anti-FIX(a) antibody heavy chain CDR1-3 sequences identified by SEQ ID NOs:52, 53 and 54, respectively, optionally comprising 1, 2 or 3 amino acid substitutions and/or deletions and/or insertions; and
      the anti-FIX(a) antibody light chain CDR1-3 sequences identified by SEQ ID NOs:56, 57 and 58, respectively, optionally comprising 1, 2 or 3 amino acid substitutions and/or deletions and/or insertions; and
      the anti-FX(a) antibody heavy chain CDR1-3 sequences identified by SEQ ID NOs:732, 733 and 734, respectively, optionally comprising 1, 2 or 3 amino acid substitutions and/or deletions and/or insertions; and
      the anti-FX(a) antibody light chain CDR1-3 sequences identified by SEQ ID NOs:736, 737 and 738, respectively, optionally comprising 1, 2 or 3 amino acid substitutions and/or deletions and/or insertions; or
   g. the anti-FIX(a) antibody heavy chain CDR1-3 sequences identified by SEQ ID NOs:52, 53 and 54, respectively, optionally comprising 1, 2 or 3 amino acid substitutions and/or deletions and/or insertions; and
      the anti-FIX(a) antibody light chain CDR1-3 sequences identified by SEQ ID NOs:56, 57 and 58, respectively, optionally comprising 1, 2 or 3 amino acid substitutions and/or deletions and/or insertions; and
      the anti-FX(a) antibody heavy chain CDR1-3 sequences identified by SEQ ID NOs:908, 909 and 910, respectively, optionally comprising 1, 2 or 3 amino acid substitutions and/or deletions and/or insertions; and
      the anti-FX(a) antibody light chain CDR1-3 sequences identified by SEQ ID NOs:912, 913 and 914, respectively, optionally comprising 1, 2 or 3 amino acid substitutions and/or deletions and/or insertions; or
   h. the anti-FIX(a) antibody heavy chain CDR1-3 sequences identified by SEQ ID NOs:52, 53 and 54, respectively, optionally comprising 1, 2 or 3 amino acid substitutions and/or deletions and/or insertions; and
      the anti-FIX(a) antibody light chain CDR1-3 sequences identified by SEQ ID NOs:56, 57 and 58, respectively, optionally comprising 1, 2 or 3 amino acid substitutions and/or deletions and/or insertions; and
      the anti-FX(a) antibody heavy chain CDR1-3 sequences identified by SEQ ID NOs:1076, 1077 and 1078, respectively, optionally comprising 1, 2 or 3 amino acid substitutions and/or deletions and/or insertions; and
      the anti-FX(a) antibody light chain CDR1-3 sequences identified by SEQ ID NOs:1080, 1081 and 1082, respectively, optionally comprising 1, 2 or 3 amino acid substitutions and/or deletions and/or insertions; or
   i. the anti-FIX(a) antibody heavy chain CDR1-3 sequences identified by SEQ ID NOs:52, 53 and 54, respectively, optionally comprising 1, 2 or 3 amino acid substitutions and/or deletions and/or insertions; and
      the anti-FIX(a) antibody light chain CDR1-3 sequences identified by SEQ ID NOs:56, 57 and 58, respectively, optionally comprising 1, 2 or 3 amino acid substitutions and/or deletions and/or insertions; and
      the anti-FX(a) antibody heavy chain CDR1-3 sequences identified by SEQ ID NOs: 484, 485 and 486, respectively, optionally comprising 1, 2 or 3 amino acid substitutions and/or deletions and/or insertions; and
      the anti-FX(a) antibody light chain CDR1-3 sequences identified by SEQ ID NOs:488, 489 and 490, respectively, optionally comprising 1, 2 or 3 amino acid substitutions and/or deletions and/or insertions; or
   j. the anti-FIX(a) antibody heavy chain CDR1-3 sequences identified by SEQ ID NOs:68, 69 and 70, respectively, optionally comprising 1, 2 or 3 amino acid substitutions and/or deletions and/or insertions; and
      the anti-FIX(a) antibody light chain CDR1-3 sequences identified by SEQ ID NOs: 72, 73 and 74, respectively, optionally comprising 1, 2 or 3 amino acid substitutions and/or deletions and/or insertions; and
      the anti-FX(a) antibody heavy chain CDR1-3 sequences identified by SEQ ID NOs:468, 469 and 470, respectively, optionally comprising 1, 2 or 3 amino acid substitutions and/or deletions and/or insertions; and
      the anti-FX(a) antibody light chain CDR1-3 sequences identified by SEQ ID NOs:472, 473 and 474, respectively, optionally comprising 1, 2 or 3 amino acid substitutions and/or deletions and/or insertions; or
   k. the anti-FIX(a) antibody heavy chain CDR1-3 sequences identified by SEQ ID NOs:68, 69 and 70, respectively, optionally comprising 1, 2 or 3 amino acid substitutions and/or deletions and/or insertions; and
      the anti-FIX(a) antibody light chain CDR1-3 sequences identified by SEQ ID NOs:72, 73 and 74, respectively, optionally comprising 1, 2 or 3 amino acid substitutions and/or deletions and/or insertions; and
      the anti-FX(a) antibody heavy chain CDR1-3 sequences identified by SEQ ID NOs:484, 485 and 486, respectively, optionally comprising 1, 2 or 3 amino acid substitutions and/or deletions and/or insertions; and
      the anti-FX(a) antibody light chain CDR1-3 sequences identified by SEQ ID NOs:488, 489 and 490, respectively, optionally comprising 1, 2 or 3 amino acid substitutions and/or deletions and/or insertions; or
   l. the anti-FIX(a) antibody heavy chain CDR1-3 sequences identified by SEQ ID NOs:1203, 1204 and 1205, respectively, optionally comprising 1, 2 or 3 amino acid substitutions and/or deletions and/or insertions; and
      the anti-FIX(a) antibody light chain CDR1-3 sequences identified by SEQ ID NOs:1207, 1208 and 1209, respectively, optionally comprising 1, 2 or 3 amino acid substitutions and/or deletions and/or insertions; and
      the anti-FX(a) antibody heavy chain CDR1-3 sequences identified by SEQ ID NOs:468, 469 and 470, respectively, optionally comprising 1, 2 or 3 amino acid substitutions and/or deletions and/or insertions; and
      the anti-FX(a) antibody light chain CDR1-3 sequences identified by SEQ ID NOs:472, 473 and 474, respectively, optionally comprising 1, 2 or 3 amino acid substitutions and/or deletions and/or insertions; or
      m. the anti-FIX(a) antibody heavy chain CDR1-3 sequences identified by SEQ ID NOs:1211, 1212 and 1213, respectively, optionally comprising 1, 2 or 3 amino acid substitutions and/or deletions and/or insertions; and
      the anti-FIX(a) antibody light chain CDR1-3 sequences identified by SEQ ID NOs:1215, 1216 and 1217, respectively, optionally comprising 1, 2 or 3 amino acid substitutions and/or deletions and/or insertions; and
      the anti-FX(a) antibody heavy chain CDR1-3 sequences identified by SEQ ID NOs:468, 469 and 470, respectively, optionally comprising 1, 2 or 3 amino acid substitutions and/or deletions and/or insertions; and
      the anti-FX(a) antibody light chain CDR1-3 sequences identified by SEQ ID NOs:472, 473 and 474, respectively, optionally comprising 1, 2 or 3 amino acid substitutions and/or deletions and/or insertions; or
   n. the anti-FIX(a) antibody heavy chain CDR1-3 sequences identified by SEQ ID NOs:1219, 1220 and 1221, respectively, optionally comprising 1, 2 or 3 amino acid substitutions and/or deletions and/or insertions; and
      the anti-FIX(a) antibody light chain CDR1-3 sequences identified by SEQ ID NOs:1223, 1224 and 1225, respectively, optionally comprising 1, 2 or 3 amino acid substitutions and/or deletions and/or insertions; and
      the anti-FX(a) antibody heavy chain CDR1-3 sequences identified by SEQ ID NOs:468, 469 and 470, respectively, optionally comprising 1, 2 or 3 amino acid substitutions and/or deletions and/or insertions; and
      the anti-FX(a) antibody light chain CDR1-3 sequences identified by SEQ ID NOs:472, 473 and 474, respectively, optionally comprising 1, 2 or 3 amino acid substitutions and/or deletions and/or insertions; or
   o. the anti-FIX(a) antibody heavy chain CDR1-3 sequences identified by SEQ ID NOs:1227, 1228 and 1229, respectively, optionally comprising 1, 2 or 3 amino acid substitutions and/or deletions and/or insertions; and
      the anti-FIX(a) antibody light chain CDR1-3 sequences identified by SEQ ID NOs:1231, 1232 and 1233, respectively, optionally comprising 1, 2 or 3 amino acid substitutions and/or deletions and/or insertions; and
      the anti-FX(a) antibody heavy chain CDR1-3 sequences identified by SEQ ID NOs:468, 469 and 470, respectively, optionally comprising 1, 2 or 3 amino acid substitutions and/or deletions and/or insertions; and
      the anti-FX(a) antibody light chain CDR1-3 sequences identified by SEQ ID NOs:472, 473 and 474, respectively, optionally comprising 1, 2 or 3 amino acid substitutions and/or deletions and/or insertions; or
   p. the anti-FIX(a) antibody heavy chain CDR1-3 sequences identified by SEQ ID NOs:1235, 1236 and 1237, respectively, optionally comprising 1, 2 or 3 amino acid substitutions and/or deletions and/or insertions; and
      the anti-FIX(a) antibody light chain CDR1-3 sequences identified by SEQ ID NOs:1239, 1240 and 1241, respectively, optionally comprising 1, 2 or 3 amino acid substitutions and/or deletions and/or insertions; and
      the anti-FX(a) antibody heavy chain CDR1-3 sequences identified by SEQ ID NOs:468, 469 and 470, respectively, optionally comprising 1, 2 or 3 amino acid substitutions and/or deletions and/or insertions; and
      the anti-FX(a) antibody light chain CDR1-3 sequences identified by SEQ ID NOs:472, 473 and 474, respectively, optionally comprising 1, 2 or 3 amino acid substitutions and/or deletions and/or insertions; or
   q. the anti-FIX(a) antibody heavy chain CDR1-3 sequences identified by SEQ ID NOs:1243, 1244 and 1245, respectively, optionally comprising 1, 2 or 3 amino acid substitutions and/or deletions and/or insertions; and
      the anti-FIX(a) antibody light chain CDR1-3 sequences identified by SEQ ID NOs:1247, 1248 and 1249, respectively, optionally comprising 1, 2 or 3 amino acid substitutions and/or deletions and/or insertions; and
      the anti-FX(a) antibody heavy chain CDR1-3 sequences identified by SEQ ID NOs:468, 469 and 470, respectively, optionally comprising 1, 2 or 3 amino acid substitutions and/or deletions and/or insertions; and
      the anti-FX(a) antibody light chain CDR1-3 sequences identified by SEQ ID NOs:472, 473 and 474, respectively, optionally comprising 1, 2 or 3 amino acid substitutions and/or deletions and/or insertions.
60. The multispecific antibody or antigen-binding fragment thereof according to embodiment
51 wherein the antibody is a bispecific antibody capable of specifically binding FIX(a) and FX(a) wherein the binding domains are those of the mAb pairs consisting of: mAb01-9933/mAb01-8174, mAb01-9933/mAb01-9772, mAb01-9978/mAb01-8174, mAb01-9978/mAb01-9772, mAb01-9985/mAb01-8174, mAb01-9985/mAb01-9772, mAb01-9994/mAb01-8174, mAb01-9994/mAb01-9772, mAb01-9985/mAb11-1431, mAb01-9985/mAb11-1434, mAb01-9985/mAb11-1457, mAb01-9985/mAb11-1480, mAb01-9985/mAb11-1121, mAb01-9985/mAb11-1125, mAb11-0173/mAb01-8174, mAb11-1204/mAb01-8174, mAb11-1495/mAb01-8174, mAb11-1501/mAb01-8174 or mAb11-1502/mAb01-8174.
61. The multispecific antibody or antigen-binding fragment thereof according to any of embodiments 51-60 wherein the antibody or antigen-binding fragment thereof is a procoagulant antibody.
62. The multispecific antibody or antigen-binding fragment thereof according to any of embodiments 51-60 wherein the antibody or antigen-binding fragment thereof is capable of increasing the procoagulant activity of FIXa.
63. The multispecific antibody or antigen-binding fragment thereof according to any of embodiments 51-60 wherein the antibody or antigen-binding fragment thereof is capable of increasing the enzymatic activity of FIXa towards FX.
64. The multispecific antibody or antigen-binding fragment thereof according to any of embodiments 51-60 wherein the antibody or antigen-binding fragment thereof is capable of functionally substituting for FVIII and/or FVIIIa.
65. The multispecific antibody or antigen-binding fragment thereof according to any of embodiments 51-64 wherein the antibody or antigen-binding fragment thereof is a bispecific antibody.
66. The multispecific antibody or antigen-binding fragment thereof according to any of embodiments 63-65 wherein the increase of the enzymatic activity of FIXa towards FX is determined in a FXa generation assay as described herein using a monovalent one-armed anti-FIX/FIXa antibody where in the stimulation index is at least 1300, 1400, 1500, 1600, 1700, 1800, 1900, 2000, 2100, 2200, 2300, 2400, 2500, 3000, 3500, 4000, 4500, 5000, 5500, 6000, 6500, 7000, 7500, 8000, 8500, 9000, 9500, 10000, 10500, 11000, 11500, 12000, 12500, 13000, 13500, 13730, 14000, 14500, 15000, 15500, 16000, 16500, 17000, 17500, 18000, 18500, 19000 or 19500 fold when measured using an one-armed antibody concentration resulting in at least 80% saturation of FIXa.
67. The multispecific antibody or antigen-binding fragment thereof according to any of embodiments 51-65 wherein said antibody or antigen-binding fragment thereof is capable of providing a mean peak thrombin (in nM) of at least 47, 48, 49, 50, 55, 60, 65, 70, 75, 80, 85, 90, 95, 100, 105 or 107 at a compound concentration of 900 nM in a TGT assay (in HA-PRP) according to example 16 herein.
68. The antibody according to any of the previous embodiments wherein the antibody isotype is IgG1, IgG2, IgG3 or IgG4 or a combination thereof.
69. A pharmaceutical composition comprising an antibody or antigen-binding fragment thereof according to any of the previous embodiments and optionally one or more pharmaceutically acceptable carrier(s).
70. The pharmaceutical composition comprising an antibody or antigen-binding fragment thereof according to embodiment 69 for the treatment of a coagulopathy or blood coagulation disorder, such as haemophilia A with or without inhibitors.
71. The antibody or antigen-binding fragment thereof or composition according to any of the previous embodiments for use in a method of treatment of a coagulopathy or blood coagulation disorder.
72. The antibody or antigen-binding fragment thereof or composition according to any of the previous embodiments for use in the treatment of haemophilia A with or without inhibitors.
73. A method of treating a subject suffering from a coagulopathy or blood coagulation disorder, comprising administering to said subject an antibody or antigen-binding fragment thereof or composition according to any of the previous embodiments.
74. A method according to embodiment 73 wherein the coagulopathy or blood coagulation disorder is haemophilia A or haemophilia A with inhibitors.
75. Use of an antibody or antigen-binding fragment thereof or composition according to any of embodiments 1-69 for the manufacture of a medicament for the treatment of a subject in need thereof.
76. Use of an antibody or antigen-binding fragment thereof or composition according to any of embodiments 1-69 for the manufacture of a medicament for the treatment of haemophilia A with or without inhibitors.
77. A eukaryotic cell which expresses the antibody or antigen-binding fragment thereof, according to any one of embodiments 1-68.
78. A kit comprising the antibody or antigen-binding fragment thereof or composition according to any of embodiments 1-69 and instructions for use.
79. The antibody or antigen-binding fragment thereof according to any of embodiments 1-25 wherein the antibody or antigen-binding fragment thereof is a component (intermediate) for use in a procoagulant multispecific antibody.
80. The antibody or antigen-binding fragment thereof according to any of embodiments 26-50 wherein the antibody or antigen-binding fragment thereof is a component (intermediate) for use in a procoagulant multispecific antibody.
81. The antibody or antigen-binding fragment thereof according to any of embodiments 1-25 wherein the antibody or antigen-binding fragment thereof is a component (intermediate) for use in the manufacture of a procoagulant multispecific antibody.
82. The antibody or antigen-binding fragment thereof according to any of embodiments 26-50 wherein the antibody or antigen-binding fragment thereof is an a component (intermediate) for use in the manufacture of a procoagulant multispecific antibody.
83. The multispecific antibody or antigen-binding fragment thereof according to any of embodiments 61-68 wherein the procoagulant activity of said antibody is improved over the multispecific antibodies disclosed in WO2018/141863.
84. The multispecific antibody or antigen-binding fragment thereof according to embodiment 83 wherein said improvement is determined using an assay as disclosed herein, such as in a FXa generation assay using monovalent one-armed (OA) anti-FIXa antibodies (as described in example 12 herein), a haemophilia A (HA) plasma TGT assay (as described in example 15 herein), in a HA-PPP TGT assay or in a HA-PRP TGT assay (as described in example 16 herein) or in a murine Tail Vein Transsection (TVT) model (as described in example 17 herein).
85. The multispecific antibody or antigen-binding fragment thereof according to embodiment 51, wherein the antibody or antigen binding fragment thereof is a bispecific antibody comprising an antibody or antigen-binding fragment thereof according to embodiment 14 and an antibody or antigen-binding fragment thereof according to embodiment 36 or 37.
86. The multispecific antibody or antigen-binding fragment thereof according to embodiment 85 wherein the stimulation of the enzymatic activity of FIXa towards FX is determined in a FXa generation assay as described in example 12 herein using a monovalent one-armed anti-FIX/FIXa antibody where in the stimulation index is at least 1300, 1400, 1500, 1600, 1700, 1800, 1900, 2000, 2100, 2200, 2300, 2400, 2500, 3000, 3500, 4000, 4500, 5000, 5500, 6000, 6500, 7000, 7500, 8000, 8500, 9000, 9500, 10000, 10500, 11000, 11500, 12000, 12500, 13000, 13500, 13730, 14000, 14500, 15000, 15500, 16000, 16500, 17000, 17500, 18000, 18500, 19000 or 19500 fold when measured using an one-armed antibody concentration resulting in at least 80% saturation of FIXa.
87. The multispecific antibody or antigen-binding fragment thereof according to any of embodiment 85 wherein said antibody or antigen-binding fragment thereof is capable of providing a mean peak thrombin (in nM) of at least 47, 48, 49, 50, 55, 60, 65, 70, 75, 80, 85, 90, 95, 100, 105 or 107 at a compound concentration of 900 nM in a TGT assay (in HA-PRP) according to example 16 herein.
88. The antibody according to any of embodiments 85-87 wherein the antibody isotype is IgG1 or IgG4.
89. A pharmaceutical composition comprising an antibody or antigen-binding fragment thereof according to any of embodiments 85-88 and optionally one or more pharmaceutically acceptable carrier(s).
90. The pharmaceutical composition comprising an antibody or antigen-binding fragment thereof according to embodiment 89 for the treatment of a coagulopathy or blood coagulation disorder, such as haemophilia A with or without inhibitors.
91. The antibody or antigen-binding fragment thereof or composition according to any of embodiments 83-90 for use in a method of treatment of a coagulopathy or blood coagulation disorder.
92. The antibody or antigen-binding fragment thereof or composition according to any of embodiments 91 for use in the treatment of haemophilia A with or without inhibitors.
93. A method of treating a subject suffering from a coagulopathy or blood coagulation disorder, comprising administering to said subject an antibody or antigen-binding fragment thereof or composition according to any of embodiments 83-90.
94. A method according to embodiment 93 wherein the coagulopathy or blood coagulation disorder is haemophilia A or haemophilia A with inhibitors.
95. Use of an antibody or antigen-binding fragment thereof or composition according to any of embodiments 83-89 for the manufacture of a medicament for the treatment of a subject in need thereof.
96. Use of an antibody or antigen-binding fragment thereof or composition according to any of embodiments 83-89 for the manufacture of a medicament for the treatment of haemophilia A with or without inhibitors.
97. A eukaryotic cell which expresses the antibody or antigen-binding fragment thereof, according to any one of embodiments 83-88.
98. A kit comprising the antibody or antigen-binding fragment thereof or composition according to any of embodiments 83-89 and instructions for use.
99. The antibody or antigen-binding fragment thereof according to any of embodiments 83-88 wherein the antibody or antigen-binding fragment capable of binding to FIX(a) is a component (intermediate) for use in a procoagulant multispecific antibody.
100. The antibody or antigen-binding fragment thereof according to any of embodiments 83-88 wherein the antibody or antigen-binding fragment thereof capable of binding to FIX(a) is a component (intermediate) for use in the manufacture of a procoagulant multispecific antibody.
101. The antibody or antigen-binding fragment thereof according to any of embodiments 83-88 wherein the antibody or antigen-binding fragment thereof is a component (intermediate) for use in the manufacture of a procoagulant multispecific antibody.
102. An injection device comprising an antibody or antigen-binding fragment thereof or composition according to any of embodiments 51-69.
103. An injection device comprising an antibody or antigen-binding fragment thereof or composition according to any of embodiments 83-90.
104. The injection device according to embodiment 102 wherein said device is a disposable and/or pre-filled and/or multi-dose device, such as a pen.
105. The injection device according to embodiment 104 wherein said device is a pre-filled pen.
106. The injection device according to embodiment 104 wherein said device is a multi-dose pen.
107. The injection device according to any of embodiments 102, 104, 105 or 106 wherein said injection device comprises a tube having a needle gauge of 20 to 36.
108. The injection device according to embodiment 103 wherein said device is a disposable and/or pre-filled and/or multi-dose device, such as a pen.
109. The injection device according to embodiment 103 wherein said device is a pre-filled pen.
110. The injection device according to embodiment 108 wherein said device is a multi-dose pen.
111. The injection device according to any of embodiments 103, 108, 109 or 110 wherein said injection device comprises a tube having a needle gauge of 20 to 36.

In some embodiments the antibodies or antigen-binding fragments thereof of the invention are procoagulant bispecific antibodies capable of binding to FIX (SEQ ID NO:1) and/or the activated form thereof (FIXa) and to FX (SEQ ID NO:2) and/or the activated form thereof (FXa).

In one embodiment the bispecific antibody (bimAb) is bimAb05-0745 wherein the anti-FIX(a) arm comprises the following CDR-sequences:

VH CDR1 (SEQ ID NO: 68):
DYAMH
VH CDR2 (SEQ ID NO: 69):
GISWKGDIGGYVDSVKG
VH CDR3 (SEQ ID NO: 70):
SYGSGSFYNAFDS
VL CDR1 (SEQ ID NO: 72):
RASQSISSWLA
VL CDR2 (SEQ ID NO: 73):
KASRLDR
VL CDR3 (SEQ ID NO: 74):
LEYSSYIRT

and wherein the anti-FX(a) arm comprises the following CDR-sequences:

VH CDR1 (SEQ ID NO: 468):
TSWIV
VH CDR2 (SEQ ID NO: 469):
MIDPSDSFTSYSPSFQG
VH CDR3 (SEQ ID NO: 470):
LHYYNSEEFDV
VL CDR1 (SEQ ID NO: 472):
RASQSVSSSYLA
VL CDR2 (SEQ ID NO: 473):
GQSSRTR
VL CDR3 (SEQ ID NO: 474):
QQFGDSQLFT

In one embodiment the bispecific antibody is bimAb05-0746 wherein the anti-FIX(a) arm comprises the following CDR-sequences:

VH CDR1 (SEQ ID NO: 44):
DYAMH
VH CDR2 (SEQ ID NO: 45):
GISWKGDIGGYVDSVKG
VH CDR3 (SEQ ID NO: 46):
SYGSGSFYNAFDS
VL CDR1 (SEQ ID NO: 48):
RASQSISSWLA
VL CDR2 (SEQ ID NO: 49):
KASRLER
VL CDR3 (SEQ ID NO: 50):
LEYSSYIRT

and wherein the anti-FX(a) arm comprises the following CDR-sequences:

VH CDR1 (SEQ ID NO: 468):
TSWIV
VH CDR2 (SEQ ID NO: 469):
MIDPSDSFTSYSPSFQG
VH CDR3 (SEQ ID NO: 470):
LHYYNSEEFDV
VL CDR1 (SEQ ID NO: 472):
RASQSVSSSYLA
VL CDR2 (SEQ ID NO: 473):
GQSSRTR
VL CDR3 (SEQ ID NO: 474):
QQFGDSQLFT

In one embodiment the bispecific antibody is bimAb05-1229 wherein the anti-FIX(a) arm comprises the following CDR-sequences:

VH CDR1 (SEQ ID NO: 4):
DYAMH
VH CDR2 (SEQ ID NO: 5):
GISWRGDIGGYVDSVKG
VH CDR3 (SEQ ID NO: 6):
SYGSGSFYNAFDS
VL CDR1 (SEQ ID NO: 8):
RASQSISSWLA
VL CDR2 (SEQ ID NO: 9):
KASRLER
VL CDR3 (SEQ ID NO: 10):
LEYSSYIRT

and wherein the anti-FX(a) arm comprises the following CDR-sequences:

VH CDR1 (SEQ ID NO: 460):
TSWIV
VH CDR2 (SEQ ID NO: 461):
MIDPSDSFTSYSPSFQG
VH CDR3 (SEQ ID NO: 462):
LHYYHSEEFDV
VL CDR1 (SEQ ID NO: 464):
RASQSVSSSYLA
VL CDR2 (SEQ ID NO: 465):
GASSRAR
VL CDR3 (SEQ ID NO: 466):
QQFGSSRLFT

In one embodiment the bispecific antibody is bimAb05-2112 wherein the anti-FIX(a) arm comprises the following CDR-sequences:

VH CDR1 (SEQ ID NO: 68):
DYAMH
VH CDR2 (SEQ ID NO: 69):
GISWKGDIGGYVDSVKG
VH CDR3 (SEQ ID NO: 70):
SYGSGSFYNAFDS
VL CDR1 (SEQ ID NO: 72):
RASQSISSWLA
VL CDR2 (SEQ ID NO: 73):
KASRLDR
VL CDR3 (SEQ ID NO: 74):
LEYSSYIRT

and wherein the anti-FX(a) arm comprises the following CDR-sequences:

VH CDR1 (SEQ ID NO: 484):
TSWIS
VH CDR2 (SEQ ID NO: 485):
MIDPSDSYTSYSPSFQG
VH CDR3 (SEQ ID NO: 486):
LHYYNSEEFDV
VL CDR1 (SEQ ID NO: 488):
RASQSVSSSYLA
VL CDR2 (SEQ ID NO: 489):
GQSSRTR
VL CDR3 (SEQ ID NO: 490):
QQYGDSQLFT

In one embodiment the bispecific antibody is bimAb05-2113 wherein the anti-FIX(a) arm comprises the following CDR-sequences:

VH CDR1 (SEQ ID NO: 36):
DYAMH
VH CDR2 (SEQ ID NO: 37):
GISWKGDIGGYVDSVKG
VH CDR3 (SEQ ID NO: 38):
SYGSGSFYNAFDS
VL CDR1 (SEQ ID NO: 40):
RASQSISSWLA
VL CDR2 (SEQ ID NO: 41):
KASKLDR
VL CDR3 (SEQ ID NO: 42):
LEYSSYIRT

and wherein the anti-FX(a) arm comprises the following CDR-sequences:

VH CDR1 (SEQ ID NO: 484):
TSWIS
VH CDR2 (SEQ ID NO: 485):
MIDPSDSYTSYSPSFQG
VH CDR3 (SEQ ID NO: 486):
LHYYNSEEFDV
VL CDR1 (SEQ ID NO: 488):
RASQSVSSSYLA
VL CDR2 (SEQ ID NO: 489):
GQSSRTR
VL CDR3 (SEQ ID NO: 490):
QQYGDSQLFT

In one embodiment the bispecific antibody is bimAb05-2114 wherein the anti-FIX(a) arm comprises the following CDR-sequences:

VH CDR1 (SEQ ID NO: 52):
DYAMH
VH CDR2 (SEQ ID NO: 53):
GISWRGDIGGYVKSVKG
VH CDR3 (SEQ ID NO: 54):
SYGSGSFYNAFDS
VL CDR1 (SEQ ID NO: 56):
RASQSISSWLA
VL CDR2 (SEQ ID NO: 57):
KASKLER
VL CDR3 (SEQ ID NO: 58):
LEYSSYIRT

and wherein the anti-FX(a) arm comprises the following CDR-sequences:

VH CDR1 (SEQ ID NO: 484):
TSWIS
VH CDR2 (SEQ ID NO: 485):
MIDPSDSYTSYSPSFQG
VH CDR3 (SEQ ID NO: 486):
LHYYNSEEFDV
VL CDR1 (SEQ ID NO: 488):
RASQSVSSSYLA
VL CDR2 (SEQ ID NO: 489):
GQSSRTR
VL CDR3 (SEQ ID NO: 490):
QQYGDSQLFT

In one embodiment the bispecific antibody is bimAb05-2115 wherein the anti-FIX(a) arm comprises the following CDR-sequences:

VH CDR1 (SEQ ID NO: 44):
DYAMH
VH CDR2 (SEQ ID NO: 45):
GISWKGDIGGYVDSVKG
VH CDR3 (SEQ ID NO: 46):
SYGSGSFYNAFDS
VL CDR1 (SEQ ID NO: 48):
RASQSISSWLA
VL CDR2 (SEQ ID NO: 49):
KASRLER
VL CDR3 (SEQ ID NO: 50):
LEYSSYIRT

and wherein the anti-FX(a) arm comprises the following CDR-sequences:

VH CDR1 (SEQ ID NO: 484):
TSWIS
VH CDR2 (SEQ ID NO: 485):
MIDPSDSYTSYSPSFQG
VH CDR3 (SEQ ID NO: 486):
LHYYNSEEFDV
VL CDR1 (SEQ ID NO: 488):
RASQSVSSSYLA
VL CDR2 (SEQ ID NO: 489):
GQSSRTR
VL CDR3 (SEQ ID NO: 490):
QQYGDSQLFT

In one embodiment the bispecific antibody is bimAb05-2375 wherein the anti-FIX(a) arm comprises the following CDR-sequences:

VH CDR1 (SEQ ID NO: 4):
DYAMH
VH CDR2 (SEQ ID NO: 5):
GISWRGDIGGYVDSVKG
VH CDR3 (SEQ ID NO: 6):
SYGSGSFYNAFDS
VL CDR1 (SEQ ID NO: 8):
RASQSISSWLA
VL CDR2 (SEQ ID NO: 9):
KASRLER
VL CDR3 (SEQ ID NO: 10):
LEYSSYIRT

and wherein the anti-FX(a) arm comprises the following CDR-sequences:

VH CDR1 (SEQ ID NO: 468):
TSWIV
VH CDR2 (SEQ ID NO: 469):
MIDPSDSFTSYSPSFQG
VH CDR3 (SEQ ID NO: 470):
LHYYNSEEFDV
VL CDR1 (SEQ ID NO: 472):
RASQSVSSSYLA
VL CDR2 (SEQ ID NO: 473):
GQSSRTR
VL CDR3 (SEQ ID NO: 474):
QQFGDSQLFT

In one embodiment the bispecific antibody is bimAb05-2379 wherein the anti-FIX(a) arm comprises the following CDR-sequences:

VH CDR1 (SEQ ID NO: 4):
DYAMH
VH CDR2 (SEQ ID NO: 5):
GISWRGDIGGYVDSVKG
VH CDR3 (SEQ ID NO: 6):
SYGSGSFYNAFDS
VL CDR1 (SEQ ID NO: 8):
RASQSISSWLA
VL CDR2 (SEQ ID NO: 9):
KASRLER
VL CDR3 (SEQ ID NO: 10):
LEYSSYIRT

and wherein the anti-FX(a) arm comprises the following CDR-sequences:

VH CDR1 (SEQ ID NO: 476):
TSWIV
VH CDR2 (SEQ ID NO: 477):
MIDPSDSFTSYSPSFQG
VH CDR3 (SEQ ID NO: 478):
LHYYNSEEFDV
VL CDR1 (SEQ ID NO: 480):
RASQSVSSSYLA
VL CDR2 (SEQ ID NO: 481):
GASSRAR
VL CDR3 (SEQ ID NO: 482):
QQFGSSRLFT

In one embodiment the bispecific antibody is bimAb05-2532 wherein the anti-FIX(a) arm comprises the following CDR-sequences:

VH CDR1 (SEQ ID NO: 12):
DYAMH
VH CDR2 (SEQ ID NO: 13):
GISWRGDIIGYVDSVKG
VH CDR3 (SEQ ID NO: 14):
SYGSGSFYNAFDS
VL CDR1 (SEQ ID NO: 16):
RASKSISSWLA
VL CDR2 (SEQ ID NO: 17):
KASRLDR
VL CDR3 (SEQ ID NO: 18):
LEYSSYIRT

and wherein the anti-FX(a) arm comprises the following CDR-sequences:

VH CDR1 (SEQ ID NO: 460):
TSWIV
VH CDR2 (SEQ ID NO: 461):
MIDPSDSFTSYSPSFQG
VH CDR3 (SEQ ID NO: 462):
LHYYHSEEFDV
VL CDR1 (SEQ ID NO: 464):
RASQSVSSSYLA
VL CDR2 (SEQ ID NO: 465):
GASSRAR
VL CDR3 (SEQ ID NO: 466):
QQFGSSRLFT

In one embodiment the bispecific antibody is bimAb05-3279 wherein the anti-FIX(a) arm comprises the following CDR-sequences:

VH CDR1 (SEQ ID NO: 36):
DYAMH
VH CDR2 (SEQ ID NO: 37):
GISWKGDIGGYVDSVKG
VH CDR3 (SEQ ID NO: 38):
SYGSGSFYNAFDS
VL CDR1 (SEQ ID NO: 40):
RASQSISSWLA
VL CDR2 (SEQ ID NO: 41):
KASKLDR
VL CDR3 (SEQ ID NO: 42):
LEYSSYIRT

and wherein the anti-FX(a) arm comprises the following CDR-sequences:

VH CDR1 (SEQ ID NO: 460):
TSWIV
VH CDR2 (SEQ ID NO: 461):
MIDPSDSFTSYSPSFQG
VH CDR3 (SEQ ID NO: 462):
LHYYHSEEFDV
VL CDR1 (SEQ ID NO: 464):
RASQSVSSSYLA
VL CDR2 (SEQ ID NO: 465):
GASSRAR
VL CDR3 (SEQ ID NO: 466):
QQFGSSRLFT

In one embodiment the bispecific antibody is bimAb05-3409 wherein the anti-FIX(a) arm comprises the following CDR-sequences:

VH CDR1 (SEQ ID NO: 44):
DYAMH
VH CDR2 (SEQ ID NO: 45):
GISWKGDIGGYVDSVKG
VH CDR3 (SEQ ID NO: 46):
SYGSGSFYNAFDS
VL CDR1 (SEQ ID NO: 48):
RASQSISSWLA
VL CDR2 (SEQ ID NO: 49):
KASRLER
VL CDR3 (SEQ ID NO: 501:
LEYSSYIRT

and wherein the anti-FX(a) arm comprises the following CDR-sequences:

VH CDR1 (SEQ ID NO: 460):
TSWIV
VH CDR2 (SEQ ID NO: 461):
MIDPSDSFTSYSPSFQG
VH CDR3 (SEQ ID NO: 462):
LHYYHSEEFDV
VL CDR1 (SEQ ID NO: 464):
RASQSVSSSYLA
VL CDR2 (SEQ ID NO: 465):
GASSRAR
VL CDR3 (SEQ ID NO: 466):
QQFGSSRLFT

In one embodiment the bispecific antibody is bimAb05-3416 wherein the anti-FIX(a) arm comprises the following CDR-sequences:

VH CDR1 (SEQ ID NO: 52):
DYAMH
VH CDR2 (SEQ ID NO: 53):
GISWRGDIGGYVKSVKG
VH CDR3 (SEQ ID NO: 54):
SYGSGSFYNAFDS
VL CDR1 (SEQ ID NO: 56):
RASQSISSWLA
VL CDR2 (SEQ ID NO: 57):
KASKLER
VL CDR3 (SEQ ID NO: 58):
LEYSSYIRT

and wherein the anti-FX(a) arm comprises the following CDR-sequences:

VH CDR1 (SEQ ID NO: 460):
TSWIV
VH CDR2 (SEQ ID NO: 461):
MIDPSDSFTSYSPSFQG
VH CDR3 (SEQ ID NO: 462):
LHYYHSEEFDV
VL CDR1 (SEQ ID NO: 464):
RASQSVSSSYLA
VL CDR2 (SEQ ID NO: 465):
GASSRAR
VL CDR3 (SEQ ID NO: 466):
QQFGSSRLFT

In one embodiment the bispecific antibody is bimAb05-3755 wherein the anti-FIX(a) arm comprises the following CDR-sequences:

VH CDR1 (SEQ ID NO: 68):
DYAMH
VH CDR2 (SEQ ID NO: 69):
GISWKGDIGGYVDSVKG
VH CDR3 (SEQ ID NO: 70):
SYGSGSFYNAFDS
VL CDR1 (SEQ ID NO: 72):
RASQSISSWLA
VL CDR2 (SEQ ID NO: 73):
KASRLDR
VL CDR3 (SEQ ID NO: 74):
LEYSSYIRT

and wherein the anti-FX(a) arm comprises the following CDR-sequences:

VH CDR1 (SEQ ID NO: 460):
TSWIV
VH CDR2 (SEQ ID NO: 461):
MIDPSDSFTSYSPSFQG
VH CDR3 (SEQ ID NO: 462):
LHYYHSEEFDV
VL CDR1 (SEQ ID NO: 464):
RASQSVSSSYLA
VL CDR2 (SEQ ID NO: 465):
GASSRAR
VL CDR3 (SEQ ID NO: 466):
QQFGSSRLFT

In one embodiment the bispecific antibody is bimAb05-3761 wherein the anti-FIX(a) arm comprises the following CDR-sequences:

VH CDR1 (SEQ ID NO: 36):
DYAMH
VH CDR2 (SEQ ID NO: 37):
GISWKGDIGGYVDSVKG
VH CDR3 (SEQ ID NO: 38):
SYGSGSFYNAFDS
VL CDR1 (SEQ ID NO: 40):
RASQSISSWLA
VL CDR2 (SEQ ID NO: 41):
KASKLDR
VL CDR3 (SEQ ID NO: 42):
LEYSSYIRT

and wherein the anti-FX(a) arm comprises the following CDR-sequences:

VH CDR1 (SEQ ID NO: 468):
TSWIV
VH CDR2 (SEQ ID NO: 469):
MIDPSDSFTSYSPSFQG
VH CDR3 (SEQ ID NO: 470):
LHYYNSEEFDV
VL CDR1 (SEQ ID NO: 472):
RASQSVSSSYLA
VL CDR2 (SEQ ID NO: 473):
GQSSRTR
VL CDR3 (SEQ ID NO: 474):
QQFGDSQLFT

In one embodiment the bispecific antibody is bimAb05-3769 wherein the anti-FIX(a) arm comprises the following CDR-sequences:

VH CDR1 (SEQ ID NO: 52):
DYAMH
VH CDR2 (SEQ ID NO: 53):
GISWRGDIGGYVKSVKG
VH CDR3 (SEQ ID NO: 54):
SYGSGSFYNAFDS
VL CDR1 (SEQ ID NO: 56):
RASQSISSWLA
VL CDR2 (SEQ ID NO: 57):
KASKLER
VL CDR3 (SEQ ID NO: 58):
LEYSSYIRT

and wherein the anti-FX(a) arm comprises the following CDR-sequences:

VH CDR1 (SEQ ID NO: 468):
TSWIV
VH CDR2 (SEQ ID NO: 469):
MIDPSDSFTSYSPSFQG
VH CDR3 (SEQ ID NO: 470):
LHYYNSEEFDV
VL CDR1 (SEQ ID NO: 472):
RASQSVSSSYLA
VL CDR2 (SEQ ID NO: 473):
GQSSRTR
VL CDR3 (SEQ ID NO: 474):
QQFGDSQLFT

In one embodiment the bispecific antibody is bimAb05-3770 wherein the anti-FIX(a) arm comprises the following CDR-sequences:

VH CDR1 (SEQ ID NO: 60):
DYAMH
VH CDR2 (SEQ ID NO: 61):
GISWRGDIGGYVKSVKG
VH CDR3 (SEQ ID NO: 62):
SYGSGSFYNAFDS
VL CDR1 (SEQ ID NO: 64):
RASQSISSWLA
VL CDR2 (SEQ ID NO: 65):
KASRLER
VL CDR3 (SEQ ID NO: 66):
LEYSSYIRT

and wherein the anti-FX(a) arm comprises the following CDR-sequences:

VH CDR1 (SEQ ID NO: 468):
TSWIV
VH CDR2 (SEQ ID NO: 469):
MIDPSDSFTSYSPSFQG
VH CDR3 (SEQ ID NO: 470):
LHYYNSEEFDV
VL CDR1 (SEQ ID NO: 472):
RASQSVSSSYLA
VL CDR2 (SEQ ID NO: 473):
GQSSRTR
VL CDR3 (SEQ ID NO: 474):
QQFGDSQLFT

In one embodiment the bispecific antibody is bimAb05-3862 wherein the anti-FIX(a) arm comprises the following CDR-sequences:

VH CDR1 (SEQ ID NO: 20):
DYAMH
VH CDR2 (SEQ ID NO: 21):
GISWRGDIGGYVDSVKG
VH CDR3 (SEQ ID NO: 22):
SYGSGSFYNAFDS
VL CDR1 (SEQ ID NO: 24):
RASQSISSWLA
VL CDR2 (SEQ ID NO: 25):
KASRLDR
VL CDR3 (SEQ ID NO: 26):
LEYSSYIRT

and wherein the anti-FX(a) arm comprises the following CDR-sequences:

VH CDR1 (SEQ ID NO: 468):
TSWIV
VH CDR2 (SEQ ID NO: 469):
MIDPSDSFTSYSPSFQG
VH CDR3 (SEQ ID NO: 470):
LHYYNSEEFDV
VL CDR1 (SEQ ID NO: 472):
RASQSVSSSYLA
VL CDR2 (SEQ ID NO: 473):
GQSSRTR
VL CDR3 (SEQ ID NO: 474):
QQFGDSQLFT

In one embodiment the bispecific antibody is bimAb05-3863 wherein the anti-FIX(a) arm comprises the following CDR-sequences:

VH CDR1 (SEQ ID NO: 28):
DYAMH
VH CDR2 (SEQ ID NO: 29):
GISWRGDIGGYVDSVKG
VH CDR3 (SEQ ID NO: 30):
SYGSGSFYNAFDS
VL CDR1 (SEQ ID NO: 32):
RASQSISSWLA
VL CDR2 (SEQ ID NO: 33):
KASRLER
VL CDR3 (SEQ ID NO: 34):
LEYSSYIRT

and wherein the anti-FX(a) arm comprises the following CDR-sequences:

VH CDR1 (SEQ ID NO: 468):
TSWIV
VH CDR2 (SEQ ID NO: 469):
MIDPSDSFTSYSPSFQG
VH CDR3 (SEQ ID NO: 470):
LHYYNSEEFDV
VL CDR1 (SEQ ID NO: 472):
RASQSVSSSYLA
VL CDR2 (SEQ ID NO: 473):
GQSSRTR
VL CDR3 (SEQ ID NO: 474):
QQFGDSQLFT

In one embodiment the bispecific antibody is bimAb05-3880 wherein the anti-FIX(a) arm comprises the following CDR-sequences:

VH CDR1 (SEQ ID NO: 76):
DYAMH
VH CDR2 (SEQ ID NO: 77):
GISWRGDIKGYVDSVKG
VH CDR3 (SEQ ID NO: 78):
SYGSGSFYNAFDS
VL CDR1 (SEQ ID NO: 80):
RASKSISSWLA
VL CDR2 (SEQ ID NO: 81):
KASRLDR
VL CDR3 (SEQ ID NO: 82):
LEYSSYIRT

and wherein the anti-FX(a) arm comprises the following CDR-sequences:

VH CDR1 (SEQ ID NO: 468):
TSWIV
VH CDR2 (SEQ ID NO: 469):
MIDPSDSFTSYSPSFQG
VH CDR3 (SEQ ID NO: 470):
LHYYNSEEFDV
VL CDR1 (SEQ ID NO: 472):
RASQSVSSSYLA
VL CDR2 (SEQ ID NO: 473):
GQSSRTR
VL CDR3 (SEQ ID NO: 474):
QQFGDSQLFT

In one embodiment the bispecific antibody is bimAb05-3886 wherein the anti-FIX(a) arm comprises the following CDR-sequences:

VH CDR1 (SEQ ID NO: 84):
DYAMH
VH CDR2 (SEQ ID NO: 85):
GISWRGDIKGYVDSVKG
VH CDR3 (SEQ ID NO: 86):
SYGSGSFYNAFDS
VL CDR1 (SEQ ID NO: 88):
RASQSISSWLA
VL CDR2 (SEQ ID NO: 89):
KASRLDR
VL CDR3 (SEQ ID NO: 901:
LEYNSYIRT

and wherein the anti-FX(a) arm comprises the following CDR-sequences:

VH CDR1 (SEQ ID NO: 468):
TSWIV
VH CDR2 (SEQ ID NO: 469):
MIDPSDSFTSYSPSFQG
VH CDR3 (SEQ ID NO: 470):
LHYYNSEEFDV
VL CDR1 (SEQ ID NO: 472):
RASQSVSSSYLA
VL CDR2 (SEQ ID NO: 473):
GQSSRTR
VL CDR3 (SEQ ID NO: 474):
QQFGDSQLFT

In one embodiment the bispecific antibody is bimAb05-3955 wherein the anti-FIX(a) arm comprises the following CDR-sequences:

VH CDR1 (SEQ ID NO: 92):
DYAMH
VH CDR2 (SEQ ID NO: 93):
GISWRGDIGGYVKSVKG
VH CDR3 (SEQ ID NO: 94):
SYGSGSFYNAFDS
VL CDR1 (SEQ ID NO: 96):
RASQSISSWLA
VL CDR2 (SEQ ID NO: 97):
KAQRLDR
VL CDR3 (SEQ ID NO: 98):
LEYSSYIRT

and wherein the anti-FX(a) arm comprises the following CDR-sequences:

VH CDR1 (SEQ ID NO: 468):
TSWIV
VH CDR2 (SEQ ID NO: 469):
MIDPSDSFTSYSPSFQG
VH CDR3 (SEQ ID NO: 470):
LHYYNSEEFDV
VL CDR1 (SEQ ID NO: 472):
RASQSVSSSYLA
VL CDR2 (SEQ ID NO: 473):
GQSSRTR
VL CDR3 (SEQ ID NO: 474):
QQFGDSQLFT

In one embodiment the bispecific antibody is bimAb05-4100 wherein the anti-FIX(a) arm comprises the following CDR-sequences:

VH CDR1 (SEQ ID NO: 100):
DYAMH
VH CDR2 (SEQ ID NO: 101):
GISWKGDIGGYVDSVKG
VH CDR3 (SEQ ID NO: 102):
SYGSGSFYNAFDS
VL CDR1 (SEQ ID NO: 104):
RASQSIKSWLA
VL CDR2 (SEQ ID NO: 105):
KASRLDR
VL CDR3 (SEQ ID NO: 106):
LEYSSYIRT

and wherein the anti-FX(a) arm comprises the following CDR-sequences:

VH CDR1 (SEQ ID NO: 468):
TSWIV
VH CDR2 (SEQ ID NO: 469):
MIDPSDSFTSYSPSFQG
VH CDR3 (SEQ ID NO: 470):
LHYYNSEEFDV
VL CDR1 (SEQ ID NO: 472):
RASQSVSSSYLA
VL CDR2 (SEQ ID NO: 473):
GQSSRTR
VL CDR3 (SEQ ID NO: 474):
QQFGDSQLFT

In one embodiment the bispecific antibody is bimAb05-4114 wherein the anti-FIX(a) arm comprises the following CDR-sequences:

VH CDR1 (SEQ ID NO: 108):
DYAMH
VH CDR2 (SEQ ID NO: 109):
GISWKGDIGGYADSVKG
VH CDR3 (SEQ ID NO: 110):
SYGSGSFYNAFDS
VL CDR1 (SEQ ID NO: 112):
RASKSISSWLA
VL CDR2 (SEQ ID NO: 113):
KASRLER
VL CDR3 (SEQ ID NO: 114):
LEYSSYIRT

and wherein the anti-FX(a) arm comprises the following CDR-sequences:

VH CDR1 (SEQ ID NO: 468):
TSWIV
VH CDR2 (SEQ ID NO: 469):
MIDPSDSFTSYSPSFQG
VH CDR3 (SEQ ID NO: 470):
LHYYNSEEFDV
VL CDR1 (SEQ ID NO: 472):
RASQSVSSSYLA
VL CDR2 (SEQ ID NO: 473):
GQSSRTR
VL CDR3 (SEQ ID NO: 474):
QQFGDSQLFT

In one embodiment the bispecific antibody is bimAb05-4121 wherein the anti-FIX(a) arm comprises the following CDR-sequences:

VH CDR1 (SEQ ID NO: 116):
DYAMH
VH CDR2 (SEQ ID NO: 117):
GISWKGDIGGYADSVKG
VH CDR3 (SEQ ID NO: 118):
SYGSGSFYNAFDS
VL CDR1 (SEQ ID NO: 120):
RASQSISSWLA
VL CDR2 (SEQ ID NO: 121):
KASRLER
VL CDR3 (SEQ ID NO: 122):
LEYSSYIRT

and wherein the anti-FX(a) arm comprises the following CDR-sequences:

VH CDR1 (SEQ ID NO: 468):
TSWIV
VH CDR2 (SEQ ID NO: 469):
MIDPSDSFTSYSPSFQG
VH CDR3 (SEQ ID NO: 470):
LHYYNSEEFDV
VL CDR1 (SEQ ID NO: 472):
RASQSVSSSYLA
VL CDR2 (SEQ ID NO: 473):
GQSSRTR
VL CDR3 (SEQ ID NO: 474):
QQFGDSQLFT

In one embodiment the bispecific antibody is bimAb05-4220 wherein the anti-FIX(a) arm comprises the following CDR-sequences:

VH CDR1 (SEQ ID NO: 212):
DYAMH
VH CDR2 (SEQ ID NO: 213):
GISWKGDIGGYADSVKG
VH CDR3 (SEQ ID NO: 214):
SYGSGSFYNAFDS
VL CDR1 (SEQ ID NO: 216):
RASQSISSWLA
VL CDR2 (SEQ ID NO: 217):
KASKLDR
VL CDR3 (SEQ ID NO: 218):
LEYSSYIRT

and wherein the anti-FX(a) arm comprises the following CDR-sequences:

VH CDR1 (SEQ ID NO: 468):
TSWIV
VH CDR2 (SEQ ID NO: 469):
MIDPSDSFTSYSPSFQG
VH CDR3 (SEQ ID NO: 470):
LHYYNSEEFDV
VL CDR1 (SEQ ID NO: 472):
RASQSVSSSYLA
VL CDR2 (SEQ ID NO: 473):
GQSSRTR
VL CDR3 (SEQ ID NO: 474):
QQFGDSQLFT

In one embodiment the bispecific antibody is bimAb05-4226 wherein the anti-FIX(a) arm comprises the following CDR-sequences:

VH CDR1 (SEQ ID NO: 220):
DYAMH
VH CDR2 (SEQ ID NO: 221):
GISWKGDIGGYADSVKG
VH CDR3 (SEQ ID NO: 222):
SYGSGSFYNAFDS
VL CDR1 (SEQ ID NO: 224):
RASQSISSWLA
VL CDR2 (SEQ ID NO: 225):
KASKLER
VL CDR3 (SEQ ID NO: 226):
LEYSSYIRT

and wherein the anti-FX(a) arm comprises the following CDR-sequences:

VH CDR1 (SEQ ID NO: 468):
TSWIV
VH CDR2 (SEQ ID NO: 469):
MIDPSDSFTSYSPSFQG
VH CDR3 (SEQ ID NO: 470):
LHYYNSEEFDV
VL CDR1 (SEQ ID NO: 472):
RASQSVSSSYLA
VL CDR2 (SEQ ID NO: 473):
GQSSRTR
VL CDR3 (SEQ ID NO: 474):
QQFGDSQLFT

In one embodiment the bispecific antibody is bimAb05-4283 wherein the anti-FIX(a) arm comprises the following CDR-sequences:

VH CDR1 (SEQ ID NO: 228):
DYAMH
VH CDR2 (SEQ ID NO: 229):
GISWKGDIGGYVDSVKG
VH CDR3 (SEQ ID NO: 230):
SYGSGSFYNAFDS
VL CDR1 (SEQ ID NO: 232):
RASQSISSWLA
VL CDR2 (SEQ ID NO: 233):
KASKLDR
VL CDR3 (SEQ ID NO: 234):
LEYSSYIRT

and wherein the anti-FX(a) arm comprises the following CDR-sequences:

VH CDR1 (SEQ ID NO: 468):
TSWIV
VH CDR2 (SEQ ID NO: 469):
MIDPSDSFTSYSPSFQG
VH CDR3 (SEQ ID NO: 470):
LHYYNSEEFDV
VL CDR1 (SEQ ID NO: 472):
RASQSVSSSYLA
VL CDR2 (SEQ ID NO: 473):
GQSSRTR
VL CDR3 (SEQ ID NO: 474):
QQFGDSQLFT

In one embodiment the bispecific antibody is bimAb05-4289 wherein the anti-FIX(a) arm comprises the following CDR-sequences:

VH CDR1 (SEQ ID NO: 236):
DYAMH
VH CDR2 (SEQ ID NO: 237):
GISWKGDIGGYVDSVKG
VH CDR3 (SEQ ID NO: 238):
SYGSGSFYNAFDS
VL CDR1 (SEQ ID NO: 240):
RASQSISSWLA
VL CDR2 (SEQ ID NO: 241):
KASKLER
VL CDR3 (SEQ ID NO: 242):
LEYSSYIRT

and wherein the anti-FX(a) arm comprises the following CDR-sequences:

VH CDR1 (SEQ ID NO: 468):
TSWIV
VH CDR2 (SEQ ID NO: 469):
MIDPSDSFTSYSPSFQG
VH CDR3 (SEQ ID NO: 470):
LHYYNSEEFDV
VL CDR1 (SEQ ID NO: 472):
RASQSVSSSYLA
VL CDR2 (SEQ ID NO: 473):
GQSSRTR
VL CDR3 (SEQ ID NO: 474):
QQFGDSQLFT

In one embodiment the bispecific antibody is bimAb05-4292 wherein the anti-FIX(a) arm comprises the following CDR-sequences:

VH CDR1 (SEQ ID NO: 252):
DYAMH
VH CDR2 (SEQ ID NO: 253):
GISWKGDIGGYVDSVKG
VH CDR3 (SEQ ID NO: 254):
SYGSGSFYNAFDS
VL CDR1 (SEQ ID NO: 256):
RASQSISSWLA
VL CDR2 (SEQ ID NO: 257):
KASKLER
VL CDR3 (SEQ ID NO: 258):
LEYSSYIRT

and wherein the anti-FX(a) arm comprises the following CDR-sequences:

VH CDR1 (SEQ ID NO: 468):
TSWIV
VH CDR2 (SEQ ID NO: 469):
MIDPSDSFTSYSPSFQG
VH CDR3 (SEQ ID NO: 470):
LHYYNSEEFDV
VL CDR1 (SEQ ID NO: 472):
RASQSVSSSYLA
VL CDR2 (SEQ ID NO: 473):
GQSSRTR
VL CDR3 (SEQ ID NO: 474):
QQFGDSQLFT

In one embodiment the bispecific antibody is bimAb05-4293 wherein the anti-FIX(a) arm comprises the following CDR-sequences:

VH CDR1 (SEQ ID NO: 244):
DYAMH
VH CDR2 (SEQ ID NO: 245):
GISWKGDIGGYVDSVKG
VH CDR3 (SEQ ID NO: 246):
SYGSGSFYNAFDS
VL CDR1 (SEQ ID NO: 248):
RASQSISSWLA
VL CDR2 (SEQ ID NO: 249):
KASKLDR
VL CDR3 (SEQ ID NO: 250):
LEYSSYIRT

and wherein the anti-FX(a) arm comprises the following CDR-sequences:

VH CDR1 (SEQ ID NO: 468):
TSWIV
VH CDR2 (SEQ ID NO: 469):
MIDPSDSFTSYSPSFQG
VH CDR3 (SEQ ID NO: 470):
LHYYNSEEFDV
VL CDR1 (SEQ ID NO: 472):
RASQSVSSSYLA
VL CDR2 (SEQ ID NO: 473):
GQSSRTR
VL CDR3 (SEQ ID NO: 474):
QQFGDSQLFT

In one embodiment the bispecific antibody is bimAb05-4387 wherein the anti-FIX(a) arm comprises the following CDR-sequences:

VH CDR1 (SEQ ID NO: 260):
DYAMH
VH CDR2 (SEQ ID NO: 261):
GISWKGDIGGYVDSVKG
VH CDR3 (SEQ ID NO: 262):
SYGSGSFYNAFDS
VL CDR1 (SEQ ID NO: 264):
RASQSISSWLA
VL CDR2 (SEQ ID NO: 265):
KASKLDR
VL CDR3 (SEQ ID NO: 266):
LEYSSYIRT

and wherein the anti-FX(a) arm comprises the following CDR-sequences:

VH CDR1 (SEQ ID NO: 468):
TSWIV
VH CDR2 (SEQ ID NO: 469):
MIDPSDSFTSYSPSFQG
VH CDR3 (SEQ ID NO: 470):
LHYYNSEEFDV
VL CDR1 (SEQ ID NO: 472):
RASQSVSSSYLA
VL CDR2 (SEQ ID NO: 473):
GQSSRTR
VL CDR3 (SEQ ID NO: 474):
QQFGDSQLFT

In one embodiment the bispecific antibody is bimAb05-4392 wherein the anti-FIX(a) arm comprises the following CDR-sequences:

VH CDR1 (SEQ ID NO: 268):
DYAMH
VH CDR2 (SEQ ID NO: 269):
GISWKGDIGGYVDSVKG
VH CDR3 (SEQ ID NO: 270):
SYGSGSFYNAFDS
VL CDR1 (SEQ ID NO: 272):
RASQSISSWLA
VL CDR2 (SEQ ID NO: 273):
KASKLER
VL CDR3 (SEQ ID NO: 274):
LEYSSYIRT

and wherein the anti-FX(a) arm comprises the following CDR-sequences:

VH CDR1 (SEQ ID NO: 468):
TSWIV
VH CDR2 (SEQ ID NO: 469):
MIDPSDSFTSYSPSFQG
VH CDR3 (SEQ ID NO: 470):
LHYYNSEEFDV
VL CDR1 (SEQ ID NO: 472):
RASQSVSSSYLA
VL CDR2 (SEQ ID NO: 473):
GQSSRTR
VL CDR3 (SEQ ID NO: 474):
QQFGDSQLFT

In one embodiment the bispecific antibody is bimAb05-4419 wherein the anti-FIX(a) arm comprises the following CDR-sequences:

VH CDR1 (SEQ ID NO: 124):
DYAMH
VH CDR2 (SEQ ID NO: 125):
GISWKGDIGGYVDSVKG
VH CDR3 (SEQ ID NO: 126):
SYGSGSFYNAFDS
VL CDR1 (SEQ ID NO: 128):
RASQSISSWLA
VL CDR2 (SEQ ID NO: 129):
KASKLDR
VL CDR3 (SEQ ID NO: 130):
LEYSSYIRT

and wherein the anti-FX(a) arm comprises the following CDR-sequences:

VH CDR1 (SEQ ID NO: 468):
TSWIV
VH CDR2 (SEQ ID NO: 469):
MIDPSDSFTSYSPSFQG
VH CDR3 (SEQ ID NO: 470):
LHYYNSEEFDV
VL CDR1 (SEQ ID NO: 472):
RASQSVSSSYLA
VL CDR2 (SEQ ID NO: 473):
GQSSRTR
VL CDR3 (SEQ ID NO: 474):
QQFGDSQLFT

In one embodiment the bispecific antibody is bimAb05-4422 wherein the anti-FIX(a) arm comprises the following CDR-sequences:

VH CDR1 (SEQ ID NO: 276):
DYAMH
VH CDR2 (SEQ ID NO: 277):
GISWKGDIGGYVDSVKG
VH CDR3 (SEQ ID NO: 278):
SYGSGSFYNAFDS
VL CDR1 (SEQ ID NO: 280):
RASQSISSWLA
VL CDR2 (SEQ ID NO: 281):
KASKLDR
VL CDR3 (SEQ ID NO: 282):
LEYSSYIRT

and wherein the anti-FX(a) arm comprises the following CDR-sequences:

VH CDR1 (SEQ ID NO: 468):
TSWIV
VH CDR2 (SEQ ID NO: 469):
MIDPSDSFTSYSPSFQG
VH CDR3 (SEQ ID NO: 470):
LHYYNSEEFDV
VL CDR1 (SEQ ID NO: 472):
RASQSVSSSYLA
VL CDR2 (SEQ ID NO: 473):
GQSSRTR
VL CDR3 (SEQ ID NO: 474):
QQFGDSQLFT

In one embodiment the bispecific antibody is bimAb05-4428 wherein the anti-FIX(a) arm comprises the following CDR-sequences:

VH CDR1 (SEQ ID NO: 284):
DYAMH
VH CDR2 (SEQ ID NO: 285):
GISWKGDIGGYVDSVKG
VH CDR3 (SEQ ID NO: 286):
SYGSGSFYNAFDS
VL CDR1 (SEQ ID NO: 288):
RASQSISSWLA
VL CDR2 (SEQ ID NO: 289):
KASKLER
VL CDR3 (SEQ ID NO: 290):
LEYSSYIRT

and wherein the anti-FX(a) arm comprises the following CDR-sequences:

VH CDR1 (SEQ ID NO: 468):
TSWIV
VH CDR2 (SEQ ID NO: 469):
MIDPSDSFTSYSPSFQG
VH CDR3 (SEQ ID NO: 470):
LHYYNSEEFDV
VL CDR1 (SEQ ID NO: 472):
RASQSVSSSYLA
VL CDR2 (SEQ ID NO: 473):
GQSSRTR
VL CDR3 (SEQ ID NO: 474):
QQFGDSQLFT

In one embodiment the bispecific antibody is bimAb05-4443 wherein the anti-FIX(a) arm comprises the following CDR-sequences:

VH CDR1 (SEQ ID NO: 300):
DYAMH
VH CDR2 (SEQ ID NO: 301):
GISWKGDIGGYVDSVKG
VH CDR3 (SEQ ID NO: 302):
SYGSGSFYNAFDS
VL CDR1 (SEQ ID NO: 304):
RASQSISSWLA
VL CDR2 (SEQ ID NO: 305):
KASKLER
VL CDR3 (SEQ ID NO: 306):
LEYSSYIRT

and wherein the anti-FX(a) arm comprises the following CDR-sequences:

VH CDR1 (SEQ ID NO: 468):
TSWIV
VH CDR2 (SEQ ID NO: 469):
MIDPSDSFTSYSPSFQG
VH CDR3 (SEQ ID NO: 470):
LHYYNSEEFDV
VL CDR1 (SEQ ID NO: 472):
RASQSVSSSYLA
VL CDR2 (SEQ ID NO: 473):
GQSSRTR
VL CDR3 (SEQ ID NO: 474):
QQFGDSQLFT

In one embodiment the bispecific antibody is bimAb05-4444 wherein the anti-FIX(a) arm comprises the following CDR-sequences:

VH CDR1 (SEQ ID NO: 292):
DYAMH
VH CDR2 (SEQ ID NO: 293):
GISWKGDIGGYVDSVKG
VH CDR3 (SEQ ID NO: 294):
SYGSGSFYNAFDS
VL CDR1 (SEQ ID NO: 296):
RASQSISSWLA
VL CDR2 (SEQ ID NO: 297):
KASKLDR
VL CDR3 (SEQ ID NO: 298):
LEYSSYIRT

and wherein the anti-FX(a) arm comprises the following CDR-sequences:

VH CDR1 (SEQ ID NO: 468):
TSWIV
VH CDR2 (SEQ ID NO: 469):
MIDPSDSFTSYSPSFQG
VH CDR3 (SEQ ID NO: 470):
LHYYNSEEFDV
VL CDR1 (SEQ ID NO: 472):
RASQSVSSSYLA
VL CDR2 (SEQ ID NO: 473):
GQSSRTR
VL CDR3 (SEQ ID NO: 474):
QQFGDSQLFT

In one embodiment the bispecific antibody is bimAb05-4601 wherein the anti-FIX(a) arm comprises the following CDR-sequences:

VH CDR1 (SEQ ID NO: 188):
DYAMH
VH CDR2 (SEQ ID NO: 189):
GISWKGDIGGYVDSVKG
VH CDR3 (SEQ ID NO: 190):
SYGSGSFYNAFDS
VL CDR1 (SEQ ID NO: 192):
RASQSISSWLA
VL CDR2 (SEQ ID NO: 193):
KASRLDR
VL CDR3 (SEQ ID NO: 194):
LEYSSYIRT

and wherein the anti-FX(a) arm comprises the following CDR-sequences:

VH CDR1 (SEQ ID NO: 468):
TSWIV
VH CDR2 (SEQ ID NO: 469):
MIDPSDSFTSYSPSFQG
VH CDR3 (SEQ ID NO: 470):
LHYYNSEEFDV
VL CDR1 (SEQ ID NO: 472):
RASQSVSSSYLA
VL CDR2 (SEQ ID NO: 473):
GQSSRTR
VL CDR3 (SEQ ID NO: 474):
QQFGDSQLFT

In one embodiment the bispecific antibody is bimAb05-4604 wherein the anti-FIX(a) arm comprises the following CDR-sequences:

VH CDR1 (SEQ ID NO: 140):
DYAMH
VH CDR2 (SEQ ID NO: 141):
GISWKGDIGGYVDSVKG
VH CDR3 (SEQ ID NO: 142):
SYGSGSFYNAFDS
VL CDR1 (SEQ ID NO: 144):
RASQSISSWLA
VL CDR2 (SEQ ID NO: 145):
KASRLDR
VL CDR3 (SEQ ID NO: 146):
LEYSSYIRT

and wherein the anti-FX(a) arm comprises the following CDR-sequences:

VH CDR1 (SEQ ID NO: 468):
TSWIV
VH CDR2 (SEQ ID NO: 469):
MIDPSDSFTSYSPSFQG
VH CDR3 (SEQ ID NO: 470):
LHYYNSEEFDV
VL CDR1 (SEQ ID NO: 472):
RASQSVSSSYLA
VL CDR2 (SEQ ID NO: 473):
GQSSRTR
VL CDR3 (SEQ ID NO: 474):
QQFGDSQLFT

In one embodiment the bispecific antibody is bimAb05-4608 wherein the anti-FIX(a) arm comprises the following CDR-sequences:

VH CDR1 (SEQ ID NO: 204):
DYAMH
VH CDR2 (SEQ ID NO: 205):
GISWKGDIGGYVDSVKG
VH CDR3 (SEQ ID NO: 206):
SYGSGSFYNAFDS
VL CDR1 (SEQ ID NO: 208):
RASQSISSWLA
VL CDR2 (SEQ ID NO: 209):
KASRLDR
VL CDR3 (SEQ ID NO: 210):
LEYSSYIRT

and wherein the anti-FX(a) arm comprises the following CDR-sequences:

VH CDR1 (SEQ ID NO: 468):
TSWIV
VH CDR2 (SEQ ID NO: 469):
MIDPSDSFTSYSPSFQG
VH CDR3 (SEQ ID NO: 470):
LHYYNSEEFDV
VL CDR1 (SEQ ID NO: 472):
RASQSVSSSYLA
VL CDR2 (SEQ ID NO: 473):
GQSSRTR
VL CDR3 (SEQ ID NO: 474):
QQFGDSQLFT

In one embodiment the bispecific antibody is bimAb05-4611 wherein the anti-FIX(a) arm comprises the following CDR-sequences:

VH CDR1 (SEQ ID NO: 180):
DYAMH
VH CDR2 (SEQ ID NO: 181):
GISWKGDIGGYVDSVKG
VH CDR3 (SEQ ID NO: 182):
SYGSGSFYNAFDS
VL CDR1 (SEQ ID NO: 184):
RASQSISSWLA
VL CDR2 (SEQ ID NO: 185):
KASRLDR
VL CDR3 (SEQ ID NO: 186):
LEYSSYIRT

and wherein the anti-FX(a) arm comprises the following CDR-sequences:

VH CDR1 (SEQ ID NO: 468):
TSWIV
VH CDR2 (SEQ ID NO: 469):
MIDPSDSFTSYSPSFQG
VH CDR3 (SEQ ID NO: 470):
LHYYNSEEFDV
VL CDR1 (SEQ ID NO: 472):
RASQSVSSSYLA
VL CDR2 (SEQ ID NO: 473):
GQSSRTR
VL CDR3 (SEQ ID NO: 474):
QQFGDSQLFT

In one embodiment the bispecific antibody is bimAb05-4612 wherein the anti-FIX(a) arm comprises the following CDR-sequences:

VH CDR1 (SEQ ID NO: 196):
DYAMH
VH CDR2 (SEQ ID NO: 197):
GISWKGDIGGYVDSVKG
VH CDR3 (SEQ ID NO: 198):
SYGSGSFYNAFDS
VL CDR1 (SEQ ID NO: 200):
RASQSISSWLA
VL CDR2 (SEQ ID NO: 201):
KASRLDR
VL CDR3 (SEQ ID NO: 202):
LEYSSYIRT

and wherein the anti-FX(a) arm comprises the following CDR-sequences:

VH CDR1 (SEQ ID NO: 468):
TSWIV
VH CDR2 (SEQ ID NO: 469):
MIDPSDSFTSYSPSFQG
VH CDR3 (SEQ ID NO: 470):
LHYYNSEEFDV
VL CDR1 (SEQ ID NO: 472):
RASQSVSSSYLA
VL CDR2 (SEQ ID NO: 473):
GQSSRTR
VL CDR3 (SEQ ID NO: 474):
QQFGDSQLFT

In one embodiment the bispecific antibody is bimAb05-4613 wherein the anti-FIX(a) arm comprises the following CDR-sequences:

VH CDR1 (SEQ ID NO: 172):
DYAMH
VH CDR2 (SEQ ID NO: 173):
GISWKGDIGGYVDSVKG
VH CDR3 (SEQ ID NO: 174):
SYGSGSFYNAFDS
VL CDR1 (SEQ ID NO: 176):
RASQSISSWLA
VL CDR2 (SEQ ID NO: 177):
KASRLDR
VL CDR3 (SEQ ID NO: 178):
LEYSSYIRT

and wherein the anti-FX(a) arm comprises the following CDR-sequences:

VH CDR1 (SEQ ID NO: 468):
TSWIV
VH CDR2 (SEQ ID NO: 469):
MIDPSDSFTSYSPSFQG
VH CDR3 (SEQ ID NO: 470):
LHYYNSEEFDV
VL CDR1 (SEQ ID NO: 472):
RASQSVSSSYLA
VL CDR2 (SEQ ID NO: 473):
GQSSRTR
VL CDR3 (SEQ ID NO: 474):
QQFGDSQLFT

In one embodiment the bispecific antibody is bimAb05-4615 wherein the anti-FIX(a) arm comprises the following CDR-sequences:

VH CDR1 (SEQ ID NO: 164):
DYAMH
VH CDR2 (SEQ ID NO: 165):
GISWKGDIGGYVDSVKG
VH CDR3 (SEQ ID NO: 166):
SYGSGSFYNAFDS
VL CDR1 (SEQ ID NO: 168):
RASQSISSWLA
VL CDR2 (SEQ ID NO: 169):
KASRLDR
VL CDR3 (SEQ ID NO: 170):
LEYSSYIRT

and wherein the anti-FX(a) arm comprises the following CDR-sequences:

VH CDR1 (SEQ ID NO: 468):
TSWIV
VH CDR2 (SEQ ID NO: 469):
MIDPSDSFTSYSPSFQG
VH CDR3 (SEQ ID NO: 470):
LHYYNSEEFDV
VL CDR1 (SEQ ID NO: 472):
RASQSVSSSYLA
VL CDR2 (SEQ ID NO: 473):
GQSSRTR
VL CDR3 (SEQ ID NO: 474):
QQFGDSQLFT

In one embodiment the bispecific antibody is bimAb05-4617 wherein the anti-FIX(a) arm comprises the following CDR-sequences:

VH CDR1 (SEQ ID NO: 148):
DYAMH
VH CDR2 (SEQ ID NO: 149):
GISWKGDIGGYVDSVKG
VH CDR3 (SEQ ID NO: 150):
SYGSGSFYNAFDS
VL CDR1 (SEQ ID NO: 152):
RASQSISSWLA
VL CDR2 (SEQ ID NO: 153):
KASRLDR
VL CDR3 (SEQ ID NO: 154):
LEYSSYIRT

and wherein the anti-FX(a) arm comprises the following CDR-sequences:

VH CDR1 (SEQ ID NO: 468):
TSWIV
VH CDR2 (SEQ ID NO: 469):
MIDPSDSFTSYSPSFQG
VH CDR3 (SEQ ID NO: 470):
LHYYNSEEFDV
VL CDR1 (SEQ ID NO: 472):
RASQSVSSSYLA
VL CDR2 (SEQ ID NO: 473):
GQSSRTR
VL CDR3 (SEQ ID NO: 474):
QQFGDSQLFT

In one embodiment the bispecific antibody is bimAb05-4618 wherein the anti-FIX(a) arm comprises the following CDR-sequences:

VH CDR1 (SEQ ID NO: 156):
DYAMH
VH CDR2 (SEQ ID NO: 157):
GISWKGDIGGYVDSVKG
VH CDR3 (SEQ ID NO: 158):
SYGSGSFYNAFDS
VL CDR1 (SEQ ID NO: 160):
RASQSISSWLA
VL CDR2 (SEQ ID NO: 161):
KASRLDR
VL CDR3 (SEQ ID NO: 162):
LEYSSYIRT

and wherein the anti-FX(a) arm comprises the following CDR-sequences:

VH CDR1 (SEQ ID NO: 468):
TSWIV
VH CDR2 (SEQ ID NO: 469):
MIDPSDSFTSYSPSFQG
VH CDR3 (SEQ ID NO: 470):
LHYYNSEEFDV
VL CDR1 (SEQ ID NO: 472):
RASQSVSSSYLA
VL CDR2 (SEQ ID NO: 473):
GQSSRTR
VL CDR3 (SEQ ID NO: 474):
QQFGDSQLFT

In one embodiment the bispecific antibody is bimAb05-4684 wherein the anti-FIX(a) arm comprises the following CDR-sequences:

VH CDR1 (SEQ ID NO: 52):
DYAMH
VH CDR2 (SEQ ID NO: 53):
GISWRGDIGGYVKSVKG
VH CDR3 (SEQ ID NO: 54):
SYGSGSFYNAFDS
VL CDR1 (SEQ ID NO: 56):
RASQSISSWLA
VL CDR2 (SEQ ID NO: 57):
KASKLER
VL CDR3 (SEQ ID NO: 58):
LEYSSYIRT

and wherein the anti-FX(a) arm comprises the following CDR-sequences:

VH CDR1 (SEQ ID NO: 500):
TSWIS
VH CDR2 (SEQ ID NO: 501):
MIDPSDSYTSYSPSFQG
VH CDR3 (SEQ ID NO: 502):
LHYYNSEEFDV
VL CDR1 (SEQ ID NO: 504):
RASQSVSSSYLA
VL CDR2 (SEQ ID NO: 505):
GQSSRTR
VL CDR3 (SEQ ID NO: 506):
QQFGDSQLFT

In one embodiment the bispecific antibody is bimAb05-4685 wherein the anti-FIX(a) arm comprises the following CDR-sequences:

VH CDR1 (SEQ ID NO: 52):
DYAMH
VH CDR2 (SEQ ID NO: 53):
GISWRGDIGGYVKSVKG
VH CDR3 (SEQ ID NO: 54):
SYGSGSFYNAFDS
VL CDR1 (SEQ ID NO: 56):
RASQSISSWLA
VL CDR2 (SEQ ID NO: 57):
KASKLER
VL CDR3 (SEQ ID NO: 58):
LEYSSYIRT

and wherein the anti-FX(a) arm comprises the following CDR-sequences:

VH CDR1 (SEQ ID NO: 508):
TSWIS
VH CDR2 (SEQ ID NO: 509):
MIDPSDSFTSYSPSFQG
VH CDR3 (SEQ ID NO: 510):
LHYYHSEEFDV
VL CDR1 (SEQ ID NO: 512):
RASQSVSSSYLA
VL CDR2 (SEQ ID NO: 513):
GQSSRTR
VL CDR3 (SEQ ID NO: 514):
QQFGDSQLFT

In one embodiment the bispecific antibody is bimAb05-4686 wherein the anti-FIX(a) arm comprises the following CDR-sequences:

VH CDR1 (SEQ ID NO: 52):
DYAMH
VH CDR2 (SEQ ID NO: 53):
GISWRGDIGGYVKSVKG
VH CDR3 (SEQ ID NO: 54):
SYGSGSFYNAFDS
VL CDR1 (SEQ ID NO: 56):
RASQSISSWLA
VL CDR2 (SEQ ID NO: 57):
KASKLER
VL CDR3 (SEQ ID NO: 58):
LEYSSYIRT

and wherein the anti-FX(a) arm comprises the following CDR-sequences:

VH CDR1 (SEQ ID NO: 516):
TSWIS
VH CDR2 (SEQ ID NO: 517):
MIDPSDSYTSYSPSFQG
VH CDR3 (SEQ ID NO: 518):
LHYYHSEEFDV
VL CDR1 (SEQ ID NO: 520):
RASQSVSSSYLA
VL CDR2 (SEQ ID NO: 521):
GQSSRTR
VL CDR3 (SEQ ID NO: 522):
QQFGDSQLFT

In one embodiment the bispecific antibody is bimAb05-4687 wherein the anti-FIX(a) arm comprises the following CDR-sequences:

VH CDR1 (SEQ ID NO: 52):
DYAMH
VH CDR2 (SEQ ID NO: 53):
GISWRGDIGGYVKSVKG
VH CDR3 (SEQ ID NO: 54):
SYGSGSFYNAFDS
VL CDR1 (SEQ ID NO: 56):
RASQSISSWLA
VL CDR2 (SEQ ID NO: 57):
KASKLER
VL CDR3 (SEQ ID NO: 58):
LEYSSYIRT

and wherein the anti-FX(a) arm comprises the following CDR-sequences:

VH CDR1 (SEQ ID NO: 524):
TSWIS
VH CDR2 (SEQ ID NO: 525):
MIDPSDSYTSYSPSFQG
VH CDR3 (SEQ ID NO: 526):
LHYYHSEEFDV
VL CDR1 (SEQ ID NO: 528):
RASQSVSSSYLA
VL CDR2 (SEQ ID NO: 529):
GQSSRTR
VL CDR3 (SEQ ID NO: 530):
QQFGDSQLFT

In one embodiment the bispecific antibody is bimAb05-4688 wherein the anti-FIX(a) arm comprises the following CDR-sequences:

VH CDR1 (SEQ ID NO: 52):
DYAMH
VH CDR2 (SEQ ID NO: 53):
GISWRGDIGGYVKSVKG
VH CDR3 (SEQ ID NO: 54):
SYGSGSFYNAFDS
VL CDR1 (SEQ ID NO: 56):
RASQSISSWLA
VL CDR2 (SEQ ID NO: 57):
KASKLER
VL CDR3 (SEQ ID NO: 58):
LEYSSYIRT

and wherein the anti-FX(a) arm comprises the following CDR-sequences:

VH CDR1 (SEQ ID NO: 532):
TSWIS
VH CDR2 (SEQ ID NO: 533):
MIDPSDSFTSYSPSFQG
VH CDR3 (SEQ ID NO: 534):
LHYYNSEEFDV
VL CDR1 (SEQ ID NO: 536):
RASQSVSSSYLA
VL CDR2 (SEQ ID NO: 537):
GQSSRTR
VL CDR3 (SEQ ID NO: 538):
QQFGDSQLFT

In one embodiment the bispecific antibody is bimAb05-4689 wherein the anti-FIX(a) arm comprises the following CDR-sequences:

VH CDR1 (SEQ ID NO: 52):
DYAMH
VH CDR2 (SEQ ID NO: 53):
GISWRGDIGGYVKSVKG
VH CDR3 (SEQ ID NO: 54):
SYGSGSFYNAFDS
VL CDR1 (SEQ ID NO: 56):
RASQSISSWLA
VL CDR2 (SEQ ID NO: 57):
KASKLER
VL CDR3 (SEQ ID NO: 58):
LEYSSYIRT

and wherein the anti-FX(a) arm comprises the following CDR-sequences:

VH CDR1 (SEQ ID NO: 540):
TSWIV
VH CDR2 (SEQ ID NO: 541):
MIDPSDSYTSYSPSFQG
VH CDR3 (SEQ ID NO: 542):
LHYYNSEEFDV
VL CDR1 (SEQ ID NO: 544):
RASQSVSSSYLA
VL CDR2 (SEQ ID NO: 545):
GQSSRTR
VL CDR3 (SEQ ID NO: 546):
QQFGDSQLFT

In one embodiment the bispecific antibody is bimAb05-4690 wherein the anti-FIX(a) arm comprises the following CDR-sequences:

VH CDR1 (SEQ ID NO: 52):
DYAMH
VH CDR2 (SEQ ID NO: 53):
GISWRGDIGGYVKSVKG
VH CDR3 (SEQ ID NO: 54):
SYGSGSFYNAFDS
VL CDR1 (SEQ ID NO: 56):
RASQSISSWLA
VL CDR2 (SEQ ID NO: 57):
KASKLER
VL CDR3 (SEQ ID NO: 58):
LEYSSYIRT

and wherein the anti-FX(a) arm comprises the following CDR-sequences:

VH CDR1 (SEQ ID NO: 556):
TSWIV
VH CDR2 (SEQ ID NO: 557):
MIDPSDSFTSYSPSFQG
VH CDR3 (SEQ ID NO: 558):
LHYYNSEEFDV
VL CDR1 (SEQ ID NO: 560):
RASQSVSSSYLA
VL CDR2 (SEQ ID NO: 561):
GQSSRTR
VL CDR3 (SEQ ID NO: 562):
QQYGDSQLFT

In one embodiment the bispecific antibody is bimAb05-4692 wherein the anti-FIX(a) arm comprises the following CDR-sequences:

VH CDR1
(SEQ ID NO: 52):
DYAMH
VH CDR2
(SEQ ID NO: 53):
GISWRGDIGGYVKSVKG
VH CDR3
(SEQ ID NO: 54):
SYGSGSFYNAFDS
VL CDR1
(SEQ ID NO: 56):
RASQSISSWLA
VL CDR2
(SEQ ID NO: 57):
KASKLER
VL CDR3
(SEQ ID NO: 58):
LEYSSYIRT

and wherein the anti-FX(a) arm comprises the following CDR-sequences:

VH CDR1
(SEQ ID NO: 564):
TSWIS
VH CDR2
(SEQ ID NO: 565):
MIDPSDSFTSYSPSFQG
VH CDR3
(SEQ ID NO: 566):
LHYYHSEEFDV
VL CDR1
(SEQ ID NO: 568):
RASQSVSSSYLA
VL CDR2
(SEQ ID NO: 569):
GQSSRTR
VL CDR3
(SEQ ID NO: 570):
QQYGDSQLFT

In one embodiment the bispecific antibody is bimAb05-4693 wherein the anti-FIX(a) arm comprises the following CDR-sequences:

VH CDR1
(SEQ ID NO: 52):
DYAMH
VH CDR2
(SEQ ID NO: 53):
GISWRGDIGGYVKSVKG
VH CDR3
(SEQ ID NO: 54):
SYGSGSFYNAFDS
VL CDR1
(SEQ ID NO: 56):
RASQSISSWLA
VL CDR2
(SEQ ID NO: 57):
KASKLER
VL CDR3
(SEQ ID NO: 58):
LEYSSYIRT

and wherein the anti-FX(a) arm comprises the following CDR-sequences:

VH CDR1
(SEQ ID NO: 572):
TSWIS
VH CDR2
(SEQ ID NO: 573):
MIDPSDSYTSYSPSFQG
VH CDR3
(SEQ ID NO: 574):
LHYYHSEEFDV
VL CDR1
(SEQ ID NO: 576):
RASQSVSSSYLA
VL CDR2
(SEQ ID NO: 577):
GQSSRTR
VL CDR3
(SEQ ID NO: 578):
QQYGDSQLFT

In one embodiment the bispecific antibody is bimAb05-4694 wherein the anti-FIX(a) arm comprises the following CDR-sequences:

VH CDR1
(SEQ ID NO: 52):
DYAMH
VH CDR2
(SEQ ID NO: 53):
GISWRGDIGGYVKSVKG
VH CDR3
(SEQ ID NO: 54):
SYGSGSFYNAFDS
VL CDR1
(SEQ ID NO: 56):
RASQSISSWLA
VL CDR2
(SEQ ID NO: 57):
KASKLER
VL CDR3
(SEQ ID NO: 58):
LEYSSYIRT

and wherein the anti-FX(a) arm comprises the following CDR-sequences:

VH CDR1
(SEQ ID NO: 580):
TSWIS
VH CDR2
(SEQ ID NO: 581):
MIDPSDSYTSYSPSFQG
VH CDR3
(SEQ ID NO: 582):
LHYYHSEEFDV
VL CDR1
(SEQ ID NO: 584):
RASQSVSSSYLA
VL CDR2
(SEQ ID NO: 585):
GQSSRTR
VL CDR3
(SEQ ID NO: 586):
QQYGDSQLFT

In one embodiment the bispecific antibody is bimAb05-4695 wherein the anti-FIX(a) arm comprises the following CDR-sequences:

VH CDR1
(SEQ ID NO: 52):
DYAMH
VH CDR2
(SEQ ID NO: 53):
GISWRGDIGGYVKSVKG
VH CDR3
(SEQ ID NO: 54):
SYGSGSFYNAFDS
VL CDR1
(SEQ ID NO: 56):
RASQSISSWLA
VL CDR2
(SEQ ID NO: 57):
KASKLER
VL CDR3
(SEQ ID NO: 58):
LEYSSYIRT

and wherein the anti-FX(a) arm comprises the following CDR-sequences:

VH CDR1
(SEQ ID NO: 492):
TSWIS
VH CDR2
(SEQ ID NO: 493):
MIDPSDSFTSYSPSFQG
VH CDR3
(SEQ ID NO: 494):
LHYYNSEEFDV
VL CDR1
(SEQ ID NO: 496):
RASQSVSSSYLA
VL CDR2
(SEQ ID NO: 497):
GQSSRTR
VL CDR3
(SEQ ID NO: 498):
QQYGDSQLFT

In one embodiment the bispecific antibody is bimAb05-4696 wherein the anti-FIX(a) arm comprises the following CDR-sequences:

VH CDR1
(SEQ ID NO: 52):
DYAMH
VH CDR2
(SEQ ID NO: 53):
GISWRGDIGGYVKSVKG
VH CDR3
(SEQ ID NO: 54):
SYGSGSFYNAFDS
VL CDR1
(SEQ ID NO: 56):
RASQSISSWLA
VL CDR2
(SEQ ID NO: 57):
KASKLER
VL CDR3
(SEQ ID NO: 58):
LEYSSYIRT

and wherein the anti-FX(a) arm comprises the following CDR-sequences:

VH CDR1
(SEQ ID NO: 588):
TSWIV
VH CDR2
(SEQ ID NO: 589):
MIDPSDSYTSYSPSFQG
VH CDR3
(SEQ ID NO: 590):
LHYYNSEEFDV
VL CDR1
(SEQ ID NO: 592):
RASQSVSSSYLA
VL CDR2
(SEQ ID NO: 593):
GQSSRTR
VL CDR3
(SEQ ID NO: 594):
QQYGDSQLFT

In one embodiment the bispecific antibody is bimAb05-4697 wherein the anti-FIX(a) arm comprises the following CDR-sequences:

VH CDR1
(SEQ ID NO: 52):
DYAMH
VH CDR2
(SEQ ID NO: 53):
GISWRGDIGGYVKSVKG
VH CDR3
(SEQ ID NO: 54):
SYGSGSFYNAFDS
VL CDR1
(SEQ ID NO: 56):
RASQSISSWLA
VL CDR2
(SEQ ID NO: 57):
KASKLER
VL CDR3
(SEQ ID NO: 58):
LEYSSYIRT

and wherein the anti-FX(a) arm comprises the following CDR-sequences:

VH CDR1
(SEQ ID NO: 596):
TSWIV
VH CDR2
(SEQ ID NO: 597):
MIDPSDSFTSYSPSFQG
VH CDR3
(SEQ ID NO: 598):
LHYYNSEEFDV
VL CDR1
(SEQ ID NO: 600):
RASQSVSSSYLA
VL CDR2
(SEQ ID NO: 601):
GASSRAR
VL CDR3
(SEQ ID NO: 602):
QQYGDSRLFT

In one embodiment the bispecific antibody is bimAb05-4698 wherein the anti-FIX(a) arm comprises the following CDR-sequences:

VH CDR1
(SEQ ID NO: 52):
DYAMH
VH CDR2
(SEQ ID NO: 53):
GISWRGDIGGYVKSVKG
VH CDR3
(SEQ ID NO: 54):
SYGSGSFYNAFDS
VL CDR1
(SEQ ID NO: 56):
RASQSISSWLA
VL CDR2
(SEQ ID NO: 57):
KASKLER
VL CDR3
(SEQ ID NO: 58):
LEYSSYIRT

and wherein the anti-FX(a) arm comprises the following CDR-sequences:

VH CDR1 (SEQ ID NO: 604):
TSWIS
VH CDR2 (SEQ ID NO: 605):
MIDPSDSYTSYSPSFQG
VH CDR3 (SEQ ID NO: 606):
LHYYNSEEFDV
VL CDR1 (SEQ ID NO: 608):
RASQSVSSSYLA
VL CDR2 (SEQ ID NO: 609):
GASSRAR
VL CDR3 (SEQ ID NO: 610):
QQYGDSRLFT

In one embodiment the bispecific antibody is bimAb05-4699 wherein the anti-FIX(a) arm comprises the following CDR-sequences:

VH CDR1 (SEQ ID NO: 52):
DYAMH
VH CDR2 (SEQ ID NO: 53):
GISWRGDIGGYVKSVKG
VH CDR3 (SEQ ID NO: 54):
SYGSGSFYNAFDS
VL CDR1 (SEQ ID NO: 56):
RASQSISSWLA
VL CDR2 (SEQ ID NO: 57):
KASKLER
VL CDR3 (SEQ ID NO: 58):
LEYSSYIRT

and wherein the anti-FX(a) arm comprises the following CDR-sequences:

VH CDR1 (SEQ ID NO: 612):
TSWIS
VH CDR2 (SEQ ID NO: 613):
MIDPSDSFTSYSPSFQG
VH CDR3 (SEQ ID NO: 614):
LHYYHSEEFDV
VL CDR1 (SEQ ID NO: 616):
RASQSVSSSYLA
VL CDR2 (SEQ ID NO: 617):
GASSRAR
VL CDR3 (SEQ ID NO: 618):
QQYGDSRLFT

In one embodiment the bispecific antibody is bimAb05-4700 wherein the anti-FIX(a) arm comprises the following CDR-sequences:

VH CDR1 (SEQ ID NO: 52):
DYAMH
VH CDR2 (SEQ ID NO: 53):
GISWRGDIGGYVKSVKG
VH CDR3 (SEQ ID NO: 54):
SYGSGSFYNAFDS
VL CDR1 (SEQ ID NO: 56):
RASQSISSWLA
VL CDR2 (SEQ ID NO: 57):
KASKLER
VL CDR3 (SEQ ID NO: 58):
LEYSSYIRT

and wherein the anti-FX(a) arm comprises the following CDR-sequences:

VH CDR1 (SEQ ID NO: 620):
TSWIS
VH CDR2 (SEQ ID NO: 621):
MIDPSDSYTSYSPSFQG
VH CDR3 (SEQ ID NO: 622):
LHYYHSEEFDV
VL CDR1 (SEQ ID NO: 624):
RASQSVSSSYLA
VL CDR2 (SEQ ID NO: 625):
GASSRAR
VL CDR3 (SEQ ID NO: 626):
QQYGDSRLFT

In one embodiment the bispecific antibody is bimAb05-4701 wherein the anti-FIX(a) arm comprises the following CDR-sequences:

VH CDR1 (SEQ ID NO: 52):
DYAMH
VH CDR2 (SEQ ID NO: 53):
GISWRGDIGGYVKSVKG
VH CDR3 (SEQ ID NO: 54):
SYGSGSFYNAFDS
VL CDR1 (SEQ ID NO: 56):
RASQSISSWLA
VL CDR2 (SEQ ID NO: 57):
KASKLER
VL CDR3 (SEQ ID NO: 58):
LEYSSYIRT

and wherein the anti-FX(a) arm comprises the following CDR-sequences:

VH CDR1 (SEQ ID NO: 628):
TSWIS
VH CDR2 (SEQ ID NO: 629):
MIDPSDSYTSYSPSFQG
VH CDR3 (SEQ ID NO: 630):
LHYYHSEEFDV
VL CDR1 (SEQ ID NO: 632):
RASQSVSSSYLA
VL CDR2 (SEQ ID NO: 633):
GASSRAR
VL CDR3 (SEQ ID NO: 634):
QQYGDSRLFT

In one embodiment the bispecific antibody is bimAb05-4702 wherein the anti-FIX(a) arm comprises the following CDR-sequences:

VH CDR1 (SEQ ID NO: 52):
DYAMH
VH CDR2 (SEQ ID NO: 53):
GISWRGDIGGYVKSVKG
VH CDR3 (SEQ ID NO: 54):
SYGSGSFYNAFDS
VL CDR1 (SEQ ID NO: 56):
RASQSISSWLA
VL CDR2 (SEQ ID NO: 57):
KASKLER
VL CDR3 (SEQ ID NO: 58):
LEYSSYIRT

and wherein the anti-FX(a) arm comprises the following CDR-sequences:

VH CDR1 (SEQ ID NO: 636):
TSWIS
VH CDR2 (SEQ ID NO: 637):
MIDPSDSFTSYSPSFQG
VH CDR3 (SEQ ID NO: 638):
LHYYNSEEFDV
VL CDR1 (SEQ ID NO: 640):
RASQSVSSSYLA
VL CDR2 (SEQ ID NO: 641):
GASSRAR
VL CDR3 (SEQ ID NO: 642):
QQYGDSRLFT

In one embodiment the bispecific antibody is bimAb05-4703 wherein the anti-FIX(a) arm comprises the following CDR-sequences:

VH CDR1 (SEQ ID NO: 52):
DYAMH
VH CDR2 (SEQ ID NO: 53):
GISWRGDIGGYVKSVKG
VH CDR3 (SEQ ID NO: 54):
SYGSGSFYNAFDS
VL CDR1 (SEQ ID NO: 56):
RASQSISSWLA
VL CDR2 (SEQ ID NO: 57):
KASKLER
VL CDR3 (SEQ ID NO: 58):
LEYSSYIRT

and wherein the anti-FX(a) arm comprises the following CDR-sequences:

VH CDR1 (SEQ ID NO: 644):
TSWIV
VH CDR2 (SEQ ID NO: 645):
MIDPSDSYTSYSPSFQG
VH CDR3 (SEQ ID NO: 646):
LHYYNSEEFDV
VL CDR1 (SEQ ID NO: 648):
RASQSVSSSYLA
VL CDR2 (SEQ ID NO: 649):
GASSRAR
VL CDR3 (SEQ ID NO: 650):
QQYGDSRLFT

In one embodiment the bispecific antibody is bimAb05-4704 wherein the anti-FIX(a) arm comprises the following CDR-sequences:

VH CDR1 (SEQ ID NO: 52):
DYAMH
VH CDR2 (SEQ ID NO: 53):
GISWRGDIGGYVKSVKG
VH CDR3 (SEQ ID NO: 54):
SYGSGSFYNAFDS
VL CDR1 (SEQ ID NO: 56):
RASQSISSWLA
VL CDR2 (SEQ ID NO: 57):
KASKLER
VL CDR3 (SEQ ID NO: 58):
LEYSSYIRT

and wherein the anti-FX(a) arm comprises the following CDR-sequences:

VH CDR1 (SEQ ID NO: 652):
TSWIV
VH CDR2 (SEQ ID NO: 653):
MIDPSDSFTSYSPSFQG
VH CDR3 (SEQ ID NO: 654):
LHYYNSEEFDV
VL CDR1 (SEQ ID NO: 656):
RASQSVSSSYLA
VL CDR2 (SEQ ID NO: 657):
GQSSRTR
VL CDR3 (SEQ ID NO: 658):
QQFGDSRLFT

In one embodiment the bispecific antibody is bimAb05-4705 wherein the anti-FIX(a) arm comprises the following CDR-sequences:

VH CDR1 (SEQ ID NO: 52):
DYAMH
VH CDR2 (SEQ ID NO: 53):
GISWRGDIGGYVKSVKG
VH CDR3 (SEQ ID NO: 54):
SYGSGSFYNAFDS
VL CDR1 (SEQ ID NO: 56):
RASQSISSWLA
VL CDR2 (SEQ ID NO: 57):
KASKLER
VL CDR3 (SEQ ID NO: 58):
LEYSSYIRT

and wherein the anti-FX(a) arm comprises the following CDR-sequences:

VH CDR1 (SEQ ID NO: 660):
TSWIS
VH CDR2 (SEQ ID NO: 661):
MIDPSDSYTSYSPSFQG
VH CDR3 (SEQ ID NO: 662):
LHYYNSEEFDV
VL CDR1 (SEQ ID NO: 664):
RASQSVSSSYLA
VL CDR2 (SEQ ID NO: 665):
GQSSRTR
VL CDR3 (SEQ ID NO: 666):
QQFGDSRLFT

In one embodiment the bispecific antibody is bimAb05-4706 wherein the anti-FIX(a) arm comprises the following CDR-sequences:

VH CDR1 (SEQ ID NO: 52):
DYAMH
VH CDR2 (SEQ ID NO: 53):
GISWRGDIGGYVKSVKG
VH CDR3 (SEQ ID NO: 54):
SYGSGSFYNAFDS
VL CDR1 (SEQ ID NO: 56):
RASQSISSWLA
VL CDR2 (SEQ ID NO: 57):
KASKLER
VL CDR3 (SEQ ID NO: 58):
LEYSSYIRT

and wherein the anti-FX(a) arm comprises the following CDR-sequences:

VH CDR1 (SEQ ID NO: 668):
TSWIS
VH CDR2 (SEQ ID NO: 669):
MIDPSDSFTSYSPSFQG
VH CDR3 (SEQ ID NO: 670):
LHYYHSEEFDV
VL CDR1 (SEQ ID NO: 672):
RASQSVSSSYLA
VL CDR2 (SEQ ID NO: 673):
GQSSRTR
VL CDR3 (SEQ ID NO: 674):
QQFGDSRLFT

In one embodiment the bispecific antibody is bimAb05-4707 wherein the anti-FIX(a) arm comprises the following CDR-sequences:

VH CDR1 (SEQ ID NO: 52):
DYAMH
VH CDR2 (SEQ ID NO: 53):
GISWRGDIGGYVKSVKG
VH CDR3 (SEQ ID NO: 54):
SYGSGSFYNAFDS
VL CDR1 (SEQ ID NO: 56):
RASQSISSWLA
VL CDR2 (SEQ ID NO: 57):
KASKLER
VL CDR3 (SEQ ID NO: 58):
LEYSSYIRT

and wherein the anti-FX(a) arm comprises the following CDR-sequences:

VH CDR1 (SEQ ID NO: 676):
TSWIS
VH CDR2 (SEQ ID NO: 677):
MIDPSDSYTSYSPSFQG
VH CDR3 (SEQ ID NO: 678):
LHYYHSEEFDV
VL CDR1 (SEQ ID NO: 680):
RASQSVSSSYLA
VL CDR2 (SEQ ID NO: 681):
GQSSRTR
VL CDR3 (SEQ ID NO: 682):
QQFGDSRLFT

In one embodiment the bispecific antibody is bimAb05-4708 wherein the anti-FIX(a) arm comprises the following CDR-sequences:

VH CDR1 (SEQ ID NO: 52):
DYAMH
VH CDR2 (SEQ ID NO: 53):
GISWRGDIGGYVKSVKG
VH CDR3 (SEQ ID NO: 54):
SYGSGSFYNAFDS
VL CDR1 (SEQ ID NO: 56):
RASQSISSWLA
VL CDR2 (SEQ ID NO: 57):
KASKLER
VL CDR3 (SEQ ID NO: 58):
LEYSSYIRT

and wherein the anti-FX(a) arm comprises the following CDR-sequences:

VH CDR1 (SEQ ID NO: 684):
TSWIS
VH CDR2 (SEQ ID NO: 685):
MIDPSDSYTSYSPSFQG
VH CDR3 (SEQ ID NO: 686):
LHYYHSEEFDV
VL CDR1 (SEQ ID NO: 688):
RASQSVSSSYLA
VL CDR2 (SEQ ID NO: 689):
GQSSRTR
VL CDR3 (SEQ ID NO: 690):
QQFGDSRLFT

In one embodiment the bispecific antibody is bimAb05-4709 wherein the anti-FIX(a) arm comprises the following CDR-sequences:

VH CDR1 (SEQ ID NO: 52):
DYAMH
VH CDR2 (SEQ ID NO: 53):
GISWRGDIGGYVKSVKG
VH CDR3 (SEQ ID NO: 54):
SYGSGSFYNAFDS
VL CDR1 (SEQ ID NO: 56):
RASQSISSWLA
VL CDR2 (SEQ ID NO: 57):
KASKLER
VL CDR3 (SEQ ID NO: 58):
LEYSSYIRT

and wherein the anti-FX(a) arm comprises the following CDR-sequences:

VH CDR1 (SEQ ID NO: 692):
TSWIS
VH CDR2 (SEQ ID NO: 693):
MIDPSDSFTSYSPSFQG
VH CDR3 (SEQ ID NO: 694):
LHYYNSEEFDV
VL CDR1 (SEQ ID NO: 696):
RASQSVSSSYLA
VL CDR2 (SEQ ID NO: 697):
GQSSRTR
VL CDR3 (SEQ ID NO: 698):
QQFGDSRLFT

In one embodiment the bispecific antibody is bimAb05-4710 wherein the anti-FIX(a) arm comprises the following CDR-sequences:

VH CDR1 (SEQ ID NO: 52):
DYAMH
VH CDR2 (SEQ ID NO: 53):
GISWRGDIGGYVKSVKG
VH CDR3 (SEQ ID NO: 54):
SYGSGSFYNAFDS
VL CDR1 (SEQ ID NO: 56):
RASQSISSWLA
VL CDR2 (SEQ ID NO: 57):
KASKLER
VL CDR3 (SEQ ID NO: 58):
LEYSSYIRT

and wherein the anti-FX(a) arm comprises the following CDR-sequences:

VH CDR1 (SEQ ID NO: 700):
TSWIV
VH CDR2 (SEQ ID NO: 701):
MIDPSDSYTSYSPSFQG
VH CDR3 (SEQ ID NO: 702):
LHYYNSEEFDV
VL CDR1 (SEQ ID NO: 704):
RASQSVSSSYLA
VL CDR2 (SEQ ID NO: 705):
GQSSRTR
VL CDR3 (SEQ ID NO: 706):
QQFGDSRLFT

In one embodiment the bispecific antibody is bimAb05-4788 wherein the anti-FIX(a) arm comprises the following CDR-sequences:

VH CDR1 (SEQ ID NO: 308):
DYAMH
VH CDR2 (SEQ ID NO: 309):
GISWKGDIGGYVDSVKG
VH CDR3 (SEQ ID NO: 310):
SYGSGSFYNAFDS
VL CDR1 (SEQ ID NO: 312):
RASQSISSWLA
VL CDR2 (SEQ ID NO: 313):
KASRLDR
VL CDR3 (SEQ ID NO: 314):
LEYSSYIRT

and wherein the anti-FX(a) arm comprises the following CDR-sequences:

VH CDR1 (SEQ ID NO: 468):
TSWIV
VH CDR2 (SEQ ID NO: 469):
MIDPSDSFTSYSPSFQG
VH CDR3 (SEQ ID NO: 470):
LHYYNSEEFDV
VL CDR1 (SEQ ID NO: 472):
RASQSVSSSYLA
VL CDR2 (SEQ ID NO: 473):
GQSSRTR
VL CDR3 (SEQ ID NO: 474):
QQFGDSQLFT

In one embodiment the bispecific antibody is bimAb05-4884 wherein the anti-FIX(a) arm comprises the following CDR-sequences:

VH CDR1 (SEQ ID NO: 316):
DYAMH
VH CDR2 (SEQ ID NO: 317):
GISWKGDIGGYVDSVKG
VH CDR3 (SEQ ID NO: 318):
SYGSGSFYNAFDS
VL CDR1 (SEQ ID NO: 320):
RASQSISSWLA
VL CDR2 (SEQ ID NO: 321):
KASRLDR
VL CDR3 (SEQ ID NO: 322):
LEYSSYIRT

and wherein the anti-FX(a) arm comprises the following CDR-sequences:

VH CDR1 (SEQ ID NO: 468):
TSWIV
VH CDR2 (SEQ ID NO: 469):
MIDPSDSFTSYSPSFQG
VH CDR3 (SEQ ID NO: 470):
LHYYNSEEFDV
VL CDR1 (SEQ ID NO: 472):
RASQSVSSSYLA
VL CDR2 (SEQ ID NO: 473):
GQSSRTR
VL CDR3 (SEQ ID NO: 474):
QQFGDSQLFT

In one embodiment the bispecific antibody is bimAb05-4895 wherein the anti-FIX(a) arm comprises the following CDR-sequences:

VH CDR1 (SEQ ID NO: 340):
DYAMH
VH CDR2 (SEQ ID NO: 341):
GISWKGDIGGYVDSVKG
VH CDR3 (SEQ ID NO: 342):
SYGSGSFYNAFDS
VL CDR1 (SEQ ID NO: 344):
RASQSIQSWLA
VL CDR2 (SEQ ID NO: 345):
KASRLDR
VL CDR3 (SEQ ID NO: 346):
LEYSSYIRT

and wherein the anti-FX(a) arm comprises the following CDR-sequences:

VH CDR1 (SEQ ID NO: 468):
TSWIV
VH CDR2 (SEQ ID NO: 469):
MIDPSDSFTSYSPSFQG
VH CDR3 (SEQ ID NO: 470):
LHYYNSEEFDV
VL CDR1 (SEQ ID NO: 472):
RASQSVSSSYLA
VL CDR2 (SEQ ID NO: 473):
GQSSRTR
VL CDR3 (SEQ ID NO: 474):
QQFGDSQLFT

In one embodiment the bispecific antibody is bimAb05-4896 wherein the anti-FIX(a) arm comprises the following CDR-sequences:

VH CDR1 (SEQ ID NO: 324):
DYAMH
VH CDR2 (SEQ ID NO: 325):
GISWKGDIGGYVDSVKG
VH CDR3 (SEQ ID NO: 326):
SYGSGSFYNAFDS
VL CDR1 (SEQ ID NO: 328):
RASQKISSWLA
VL CDR2 (SEQ ID NO: 329):
KASRLDR
VL CDR3 (SEQ ID NO: 330):
LEYSSYIRT

and wherein the anti-FX(a) arm comprises the following CDR-sequences:

VH CDR1 (SEQ ID NO: 468):
TSWIV
VH CDR2 (SEQ ID NO: 469):
MIDPSDSFTSYSPSFQG
VH CDR3 (SEQ ID NO: 470):
LHYYNSEEFDV
VL CDR1 (SEQ ID NO: 472):
RASQSVSSSYLA
VL CDR2 (SEQ ID NO: 473):
GQSSRTR
VL CDR3 (SEQ ID NO: 474):
QQFGDSQLFT

In one embodiment the bispecific antibody is bimAb05-4898 wherein the anti-FIX(a) arm comprises the following CDR-sequences:

VH CDR1 (SEQ ID NO: 332):
DYAMH
VH CDR2 (SEQ ID NO: 333):
GISWKGDIGGYVDSVKG
VH CDR3 (SEQ ID NO: 334):
SYGSGSFYNAFDS
VL CDR1 (SEQ ID NO: 336):
RASQQISSWLA
VL CDR2 (SEQ ID NO: 337):
KASRLDR
VL CDR2 (SEQ ID NO: 338):
LEYSSYIRT

and wherein the anti-FX(a) arm comprises the following CDR-sequences:

VH CDR1 (SEQ ID NO: 468):
TSWIV
VH CDR2 (SEQ ID NO: 469):
MIDPSDSFTSYSPSFQG
VH CDR3 (SEQ ID NO: 470):
LHYYNSEEFDV
VL CDR1 (SEQ ID NO: 472):
RASQSVSSSYLA
VL CDR2 (SEQ ID NO: 473):
GQSSRTR
VL CDR3 (SEQ ID NO: 474):
QQFGDSQLFT

In one embodiment the bispecific antibody is bimAb05-4903 wherein the anti-FIX(a) arm comprises the following CDR-sequences:

VH CDR1 (SEQ ID NO: 356):
DYAMH
VH CDR2 (SEQ ID NO: 357):
GISWKGDIGGYVDSVKG
VH CDR3 (SEQ ID NO: 358):
SYGSGSFYNAFDS
VL CDR1 (SEQ ID NO: 360):
RASQSISSWLA
VL CDR2 (SEQ ID NO: 361):
KASRLDR
VL CDR3 (SEQ ID NO: 362):
LEYSSYIRT

and wherein the anti-FX(a) arm comprises the following CDR-sequences:

VH CDR1 (SEQ ID NO: 468):
TSWIV
VH CDR2 (SEQ ID NO: 469):
MIDPSDSFTSYSPSFQG
VH CDR3 (SEQ ID NO: 470):
LHYYNSEEFDV
VL CDR1 (SEQ ID NO: 472):
RASQSVSSSYLA
VL CDR2 (SEQ ID NO: 473):
GQSSRTR
VL CDR3 (SEQ ID NO: 474):
QQFGDSQLFT

In one embodiment the bispecific antibody is bimAb05-4906 wherein the anti-FIX(a) arm comprises the following CDR-sequences:

VH CDR1 (SEQ ID NO: 348):
DYAMH
VH CDR2 (SEQ ID NO: 349):
GISWKGDIGGYVDSVKG
VH CDR3 (SEQ ID NO: 350):
SYGSGSFYNAFDS
VL CDR1 (SEQ ID NO: 352):
RASQSISSWLA
VL CDR2 (SEQ ID NO: 353):
KASRLDK
VL CDR3 (SEQ ID NO: 354):
LEYSSYIRT

and wherein the anti-FX(a) arm comprises the following CDR-sequences:

VH CDR1 (SEQ ID NO: 468):
TSWIV
VH CDR2 (SEQ ID NO: 469):
MIDPSDSFTSYSPSFQG
VH CDR3 (SEQ ID NO: 470):
LHYYNSEEFDV
VL CDR1 (SEQ ID NO: 472):
RASQSVSSSYLA
VL CDR2 (SEQ ID NO: 473):
GQSSRTR
VL CDR3 (SEQ ID NO: 474):
QQFGDSQLFT

In one embodiment the bispecific antibody is bimAb05-4910 wherein the anti-FIX(a) arm comprises the following CDR-sequences:

VH CDR1 (SEQ ID NO: 364):
DYAMH
VH CDR2 (SEQ ID NO: 365):
GISWKGDIGGYVDSVKG
VH CDR3 (SEQ ID NO: 366):
SYGSGSFYNAFDS
VL CDR1 (SEQ ID NO: 368):
RASQSISSWLA
VL CDR2 (SEQ ID NO: 369):
KASRLDR
VL CDR3 (SEQ ID NO: 370):
LEYSSYIRT

and wherein the anti-FX(a) arm comprises the following CDR-sequences:

VH CDR1 (SEQ ID NO: 468):
TSWIV
VH CDR2 (SEQ ID NO: 469):
MIDPSDSFTSYSPSFQG
VH CDR3 (SEQ ID NO: 470):
LHYYNSEEFDV
VL CDR1 (SEQ ID NO: 472):
RASQSVSSSYLA
VL CDR2 (SEQ ID NO: 473):
GQSSRTR
VL CDR3 (SEQ ID NO: 474):
QQFGDSQLFT

In one embodiment the bispecific antibody is bimAb05-4914 wherein the anti-FIX(a) arm comprises the following CDR-sequences:

VH CDR1 (SEQ ID NO: 372):
DYAMH
VH CDR2 (SEQ ID NO: 373):
GISWKGDIGGYVDSVKG
VH CDR3 (SEQ ID NO: 374):
SYGSGSFYNAFDS
VL CDR1 (SEQ ID NO: 376):
RASQSISSWLA
VL CDR2 (SEQ ID NO: 377):
KASRLDR
VL CDR3 (SEQ ID NO: 378):
LEYSSYIRT

and wherein the anti-FX(a) arm comprises the following CDR-sequences:

VH CDR1 (SEQ ID NO: 468):
TSWIV
VH CDR2 (SEQ ID NO: 469):
MIDPSDSFTSYSPSFQG
VH CDR3 (SEQ ID NO: 470):
LHYYNSEEFDV
VL CDR1 (SEQ ID NO: 472):
RASQSVSSSYLA
VL CDR2 (SEQ ID NO: 473):
GQSSRTR
VL CDR3 (SEQ ID NO: 474):
QQFGDSQLFT

In one embodiment the bispecific antibody is bimAb05-4915 wherein the anti-FIX(a) arm comprises the following CDR-sequences:

VH CDR1 (SEQ ID NO: 380):
DYAMH
VH CDR2 (SEQ ID NO: 381):
GISWKGDIGGYVDSVKG
VH CDR3 (SEQ ID NO: 382):
SYGSGSFYNAFDS
VL CDR1 (SEQ ID NO: 384):
RASQSISSWLA
VL CDR2 (SEQ ID NO: 385):
KASRLDR
VL CDR3 (SEQ ID NO: 386):
LEYSSYIRT

and wherein the anti-FX(a) arm comprises the following CDR-sequences:

VH CDR1 (SEQ ID NO: 468):
TSWIV
VH CDR2 (SEQ ID NO: 469):
MIDPSDSFTSYSPSFQG
VH CDR3 (SEQ ID NO: 470):
LHYYNSEEFDV
VL CDR1 (SEQ ID NO: 472):
RASQSVSSSYLA
VL CDR2 (SEQ ID NO: 473):
GQSSRTR
VL CDR3 (SEQ ID NO: 474):
QQFGDSQLFT

In one embodiment the bispecific antibody is bimAb05-4919 wherein the anti-FIX(a) arm comprises the following CDR-sequences:

VH CDR1 (SEQ ID NO: 388):
DYAMH
VH CDR2 (SEQ ID NO: 389):
GISWKGDIGGYVDSVKG
VH CDR3 (SEQ ID NO: 390):
SYGSGSFYNAFDS
VL CDR1 (SEQ ID NO: 392):
RASQSISSWLA
VL CDR2 (SEQ ID NO: 393):
KASRLDR
VL CDR3 (SEQ ID NO: 394):
LEYQSYIRT

and wherein the anti-FX(a) arm comprises the following CDR-sequences:

VH CDR1 (SEQ ID NO: 468):
TSWIV
VH CDR2 (SEQ ID NO: 469):
MIDPSDSFTSYSPSFQG
VH CDR3 (SEQ ID NO: 470):
LHYYNSEEFDV
VL CDR1 (SEQ ID NO: 472):
RASQSVSSSYLA
VL CDR2 (SEQ ID NO: 473):
GQSSRTR
VL CDR3 (SEQ ID NO: 474):
QQFGDSQLFT

In one embodiment the bispecific antibody is bimAb05-4920 wherein the anti-FIX(a) arm comprises the following CDR-sequences:

VH CDR1 (SEQ ID NO: 404):
DYAMH
VH CDR2 (SEQ ID NO: 405):
GISWKGDIGGYVDSVKG
VH CDR3 (SEQ ID NO: 406):
SYGSGSFYNAFDS
VL CDR1 (SEQ ID NO: 408):
RASQSISSWLA
VL CDR2 (SEQ ID NO: 409):
KASRLDR
VL CDR3 (SEQ ID NO: 410):
LEYSSWIRT

and wherein the anti-FX(a) arm comprises the following CDR-sequences:

VH CDR1 (SEQ ID NO: 468):
TSWIV
VH CDR2 (SEQ ID NO: 469):
MIDPSDSFTSYSPSFQG
VH CDR3 (SEQ ID NO: 470):
LHYYNSEEFDV
VL CDR1 (SEQ ID NO: 472):
RASQSVSSSYLA
VL CDR2 (SEQ ID NO: 473):
GQSSRTR
VL CDR3 (SEQ ID NO: 474):
QQFGDSQLFT

In one embodiment the bispecific antibody is bimAb05-4921 wherein the anti-FIX(a) arm comprises the following CDR-sequences:

VH CDR1 (SEQ ID NO: 396):
DYAMH
VH CDR2 (SEQ ID NO: 397):
GISWKGDIGGYVDSVKG
VH CDR3 (SEQ ID NO: 398):
SYGSGSFYNAFDS
VL CDR1 (SEQ ID NO: 400):
RASQSISSWLA
VL CDR2 (SEQ ID NO: 401):
KASRLDR
VL CDR3 (SEQ ID NO: 402):
LEYKSYIRT

and wherein the anti-FX(a) arm comprises the following CDR-sequences:

VH CDR1 (SEQ ID NO: 468):
TSWIV
VH CDR2 (SEQ ID NO: 469):
MIDPSDSFTSYSPSFQG
VH CDR3 (SEQ ID NO: 470):
LHYYNSEEFDV
VL CDR1 (SEQ ID NO: 472):
RASQSVSSSYLA
VL CDR2 (SEQ ID NO: 473):
GQSSRTR
VL CDR3 (SEQ ID NO: 474):
QQFGDSQLFT

In one embodiment the bispecific antibody is bimAb05-4924 wherein the anti-FIX(a) arm comprises the following CDR-sequences:

VH CDR1 (SEQ ID NO: 420):
DYAMH
VH CDR2 (SEQ ID NO: 421):
GISWKGDIGGYVDSVKG
VH CDR3 (SEQ ID NO: 422):
SYGSGSFYNAFDS
VL CDR1 (SEQ ID NO: 424):
RASQSISSWLA
VL CDR2 (SEQ ID NO: 425):
KASRLDR
VL CDR3 (SEQ ID NO: 426):
LEYNSYIRT

and wherein the anti-FX(a) arm comprises the following CDR-sequences:

VH CDR1 (SEQ ID NO: 468):
TSWIV
VH CDR2 (SEQ ID NO: 469):
MIDPSDSFTSYSPSFQG
VH CDR3 (SEQ ID NO: 470):
LHYYNSEEFDV
VL CDR1 (SEQ ID NO: 472):
RASQSVSSSYLA
VL CDR2 (SEQ ID NO: 473):
GQSSRTR
VL CDR3 (SEQ ID NO: 474):
QQFGDSQLFT

In one embodiment the bispecific antibody is bimAb05-4927 wherein the anti-FIX(a) arm comprises the following CDR-sequences:

VH CDR1 (SEQ ID NO: 412):
DYAMH
VH CDR2 (SEQ ID NO: 413):
GISWKGDIGGYVDSVKG
VH CDR3 (SEQ ID NO: 414):
SYGSGSFYNAFDS
VL CDR1 (SEQ ID NO: 416):
RASQSISSWLA
VL CDR2 (SEQ ID NO: 417):
KASRLDR
VL CDR3 (SEQ ID NO: 418):
LEYRSYIRT

and wherein the anti-FX(a) arm comprises the following CDR-sequences:

VH CDR1 (SEQ ID NO: 468):
TSWIV
VH CDR2 (SEQ ID NO: 469):
MIDPSDSFTSYSPSFQG
VH CDR3 (SEQ ID NO: 470):
LHYYNSEEFDV
VL CDR1 (SEQ ID NO: 472):
RASQSVSSSYLA
VL CDR2 (SEQ ID NO: 473):
GQSSRTR
VL CDR3 (SEQ ID NO: 474):
QQFGDSQLFT

In one embodiment the bispecific antibody is bimAb05-5092 wherein the anti-FIX(a) arm comprises the following CDR-sequences:

VH CDR1 (SEQ ID NO: 428):
DYAMH
VH CDR2 (SEQ ID NO: 429):
GISWKGDIGGYVDSVKG
VH CDR3 (SEQ ID NO: 430):
SYGSGSFYNAFDS
VL CDR1 (SEQ ID NO: 432):
RASQSISSWLA
VL CDR2 (SEQ ID NO: 433):
KASKLDR
VL CDR3 (SEQ ID NO: 434):
LEYSSYIRT

and wherein the anti-FX(a) arm comprises the following CDR-sequences:

VH CDR1 (SEQ ID NO: 468):
TSWIV
VH CDR2 (SEQ ID NO: 469):
MIDPSDSFTSYSPSFQG
VH CDR3 (SEQ ID NO: 470):
LHYYNSEEFDV
VL CDR1 (SEQ ID NO: 472):
RASQSVSSSYLA
VL CDR2 (SEQ ID NO: 473):
GQSSRTR
VL CDR3 (SEQ ID NO: 474):
QQFGDSQLFT

In one embodiment the bispecific antibody is bimAb05-5095 wherein the anti-FIX(a) arm comprises the following CDR-sequences:

VH CDR1 (SEQ ID NO: 132):
DYAMH
VH CDR2 (SEQ ID NO: 133):
GISWKGDIGGYVDSVKG
VH CDR3 (SEQ ID NO: 134):
SYGSGSFYNAFDS
VL CDR1 (SEQ ID NO: 136):
RASQSISSWLA
VL CDR2 (SEQ ID NO: 137):
KASRLDR
VL CDR3 (SEQ ID NO: 138):
LEYSSYIRT

and wherein the anti-FX(a) arm comprises the following CDR-sequences:

VH CDR1 (SEQ ID NO: 468):
TSWIV
VH CDR2 (SEQ ID NO: 469):
MIDPSDSFTSYSPSFQG
VH CDR3 (SEQ ID NO: 470):
LHYYNSEEFDV
VL CDR1 (SEQ ID NO: 472):
RASQSVSSSYLA
VL CDR2 (SEQ ID NO: 473):
GQSSRTR
VL CDR3 (SEQ ID NO: 474):
QQFGDSQLFT

In one embodiment the bispecific antibody is bimAb05-5204 wherein the anti-FIX(a) arm comprises the following CDR-sequences:

VH CDR1 (SEQ ID NO: 436):
DYAMH
VH CDR2 (SEQ ID NO: 437):
GISWRGDIGGYVDSVKG
VH CDR3 (SEQ ID NO: 438):
SYGSGSFYNAFDS
VL CDR1 (SEQ ID NO: 440):
RASQSISSWLA
VL CDR2 (SEQ ID NO: 441):
KASKLDR
VL CDR3 (SEQ ID NO: 442):
LEYSSYIRT

and wherein the anti-FX(a) arm comprises the following CDR-sequences:

VH CDR1 (SEQ ID NO: 468):
TSWIV
VH CDR2 (SEQ ID NO: 469):
MIDPSDSFTSYSPSFQG
VH CDR3 (SEQ ID NO: 470):
LHYYNSEEFDV
VL CDR1 (SEQ ID NO: 472):
RASQSVSSSYLA
VL CDR2 (SEQ ID NO: 473):
GQSSRTR
VL CDR3 (SEQ ID NO: 474):
QQFGDSQLFT

In one embodiment the bispecific antibody is bimAb05-5205 wherein the anti-FIX(a) arm comprises the following CDR-sequences:

VH CDR1 (SEQ ID NO: 444):
DYAMH
VH CDR2 (SEQ ID NO: 445):
GISWRGDIGGYVDSVKG
VH CDR3 (SEQ ID NO: 446):
SYGSGSFYNAFDS
VL CDR1 (SEQ ID NO: 448):
RASQSISSWLA
VL CDR2 (SEQ ID NO: 449):
KASRLER
VL CDR3 (SEQ ID NO: 450):
LEYSSYIRT

and wherein the anti-FX(a) arm comprises the following CDR-sequences:

VH CDR1 (SEQ ID NO: 468):
TSWIV
VH CDR2 (SEQ ID NO: 469):
MIDPSDSFTSYSPSFQG
VH CDR3 (SEQ ID NO: 470):
LHYYNSEEFDV
VL CDR1 (SEQ ID NO: 472):
RASQSVSSSYLA
VL CDR2 (SEQ ID NO: 473):
GQSSRTR
VL CDR3 (SEQ ID NO: 474):
QQFGDSQLFT

In one embodiment the bispecific antibody is bimAb05-5240 wherein the anti-FIX(a) arm comprises the following CDR-sequences:

VH CDR1 (SEQ ID NO: 452):
DYAMH
VH CDR2 (SEQ ID NO: 453):
GISWKGDIGGYVDSVKG
VH CDR3 (SEQ ID NO: 454):
SYGSGSFYNAFDS
VL CDR1 (SEQ ID NO: 456):
RASQSISSWLA
VL CDR2 (SEQ ID NO: 457):
KASRLDR
VL CDR3 (SEQ ID NO: 458):
LEYSSYIRT

and wherein the anti-FX(a) arm comprises the following CDR-sequences:

VH CDR1 (SEQ ID NO: 468):
TSWIV
VH CDR2 (SEQ ID NO: 469):
MIDPSDSFTSYSPSFQG
VH CDR3 (SEQ ID NO: 470):
LHYYNSEEFDV
VL CDR1 (SEQ ID NO: 472):
RASQSVSSSYLA
VL CDR2 (SEQ ID NO: 473):
GQSSRTR
VL CDR3 (SEQ ID NO: 474):
QQFGDSQLFT

In one embodiment the bispecific antibody is bimAb05-5339 wherein the anti-FIX(a) arm comprises the following CDR-sequences:

VH CDR1 (SEQ ID NO: 52):
DYAMH
VH CDR2 (SEQ ID NO: 53):
GISWRGDIGGYVKSVKG
VH CDR3 (SEQ ID NO: 54):
SYGSGSFYNAFDS
VL CDR1 (SEQ ID NO: 56):
RASQSISSWLA
VL CDR2 (SEQ ID NO: 57):
KASKLER
VL CDR3 (SEQ ID NO: 581:
LEYSSYIRT

and wherein the anti-FX(a) arm comprises the following CDR-sequences:

VH CDR1 (SEQ ID NO: 772):
TSWIS
VH CDR2 (SEQ ID NO: 773):
MIDPSDSFTSYSPSFQG
VH CDR3 (SEQ ID NO: 774):
LHYYHSEEFDV
VL CDR1 (SEQ ID NO: 776):
RASQSVSSSYLA
VL CDR2 (SEQ ID NO: 777):
GQSSRTR
VL CDR3 (SEQ ID NO: 778):
QQFGSSQLFT

In one embodiment the bispecific antibody is bimAb05-5340 wherein the anti-FIX(a) arm comprises the following CDR-sequences:

VH CDR1 (SEQ ID NO: 52):
DYAMH
VH CDR2 (SEQ ID NO: 53):
GISWRGDIGGYVKSVKG
VH CDR3 (SEQ ID NO: 54):
SYGSGSFYNAFDS
VL CDR1 (SEQ ID NO: 56):
RASQSISSWLA
VL CDR2 (SEQ ID NO: 57):
KASKLER
VL CDR3 (SEQ ID NO: 58):
LEYSSYIRT

and wherein the anti-FX(a) arm comprises the following CDR-sequences:

VH CDR1 (SEQ ID NO: 708):
TSWIV
VH CDR2 (SEQ ID NO: 709):
MIDPSDSFTSYSPSFQG
VH CDR3 (SEQ ID NO: 710):
LHYYNSEEFDV
VL CDR1 (SEQ ID NO: 712):
RASQSVSSSYLA
VL CDR2 (SEQ ID NO: 713):
GQSSRTR
VL CDR3 (SEQ ID NO: 714):
QQFGSSQLFT

In one embodiment the bispecific antibody is bimAb05-5341 wherein the anti-FIX(a) arm comprises the following CDR-sequences:

VH CDR1 (SEQ ID NO: 52):
DYAMH
VH CDR2 (SEQ ID NO: 53):
GISWRGDIGGYVKSVKG
VH CDR3 (SEQ ID NO: 54):
SYGSGSFYNAFDS
VL CDR1 (SEQ ID NO: 56):
RASQSISSWLA
VL CDR2 (SEQ ID NO: 57):
KASKLER
VL CDR3 (SEQ ID NO: 58):
LEYSSYIRT

and wherein the anti-FX(a) arm comprises the following CDR-sequences:

VH CDR1 (SEQ ID NO: 780):
TSWIS
VH CDR2 (SEQ ID NO: 781):
MIDPSDSYTSYSPSFQG
VH CDR3 (SEQ ID NO: 782):
LHYYHSEEFDV
VL CDR1 (SEQ ID NO: 784):
RASQSVSSSYLA
VL CDR2 (SEQ ID NO: 785):
GQSSRTR
VL CDR3 (SEQ ID NO: 786):
QQFGSSQLFT

In one embodiment the bispecific antibody is bimAb05-5342 wherein the anti-FIX(a) arm comprises the following CDR-sequences:

VH CDR1 (SEQ ID NO: 52):
DYAMH
VH CDR2 (SEQ ID NO: 53):
GISWRGDIGGYVKSVKG
VH CDR3 (SEQ ID NO: 54):
SYGSGSFYNAFDS
VL CDR1 (SEQ ID NO: 56):
RASQSISSWLA
VL CDR2 (SEQ ID NO: 57):
KASKLER
VL CDR3 (SEQ ID NO: 58):
LEYSSYIRT

and wherein the anti-FX(a) arm comprises the following CDR-sequences:

VH CDR1 (SEQ ID NO: 764):
TSWIS
VH CDR2 (SEQ ID NO: 765):
MIDPSDSYTSYSPSFQG
VH CDR3 (SEQ ID NO: 766):
LHYYNSEEFDV
VL CDR1 (SEQ ID NO: 768):
RASQSVSSSYLA
VL CDR2 (SEQ ID NO: 769):
GQSSRTR
VL CDR3 (SEQ ID NO: 770):
QQFGSSQLFT

In one embodiment the bispecific antibody is bimAb05-5343 wherein the anti-FIX(a) arm comprises the following CDR-sequences:

VH CDR1 (SEQ ID NO: 52):
DYAMH
VH CDR2 (SEQ ID NO: 53):
GISWRGDIGGYVKSVKG
VH CDR3 (SEQ ID NO: 54):
SYGSGSFYNAFDS
VL CDR1 (SEQ ID NO: 56):
RASQSISSWLA
VL CDR2 (SEQ ID NO: 57):
KASKLER
VL CDR3 (SEQ ID NO: 58):
LEYSSYIRT

and wherein the anti-FX(a) arm comprises the following CDR-sequences:

VH CDR1 (SEQ ID NO: 788):
TSWIS
VH CDR2 (SEQ ID NO: 789):
MIDPSDSYTSYSPSFQG
VH CDR3 (SEQ ID NO: 790):
LHYYHSEEFDV
VL CDR1 (SEQ ID NO: 792):
RASQSVSSSYLA
VL CDR2 (SEQ ID NO: 793):
GQSSRTR
VL CDR3 (SEQ ID NO: 794):
QQFGSSQLFT

In one embodiment the bispecific antibody is bimAb05-5344 wherein the anti-FIX(a) arm comprises the following CDR-sequences:

VH CDR1 (SEQ ID NO: 52):
DYAMH
VH CDR2 (SEQ ID NO: 53):
GISWRGDIGGYVKSVKG
VH CDR3 (SEQ ID NO: 54):
SYGSGSFYNAFDS
VL CDR1 (SEQ ID NO: 56):
RASQSISSWLA
VL CDR2 (SEQ ID NO: 57):
KASKLER
VL CDR3 (SEQ ID NO: 58):
LEYSSYIRT

and wherein the anti-FX(a) arm comprises the following CDR-sequences:

VH CDR1 (SEQ ID NO: 796):
TSWIS
VH CDR2 (SEQ ID NO: 797):
MIDPSDSFTSYSPSFQG
VH CDR3 (SEQ ID NO: 798):
LHYYNSEEFDV
VL CDR1 (SEQ ID NO: 800):
RASQSVSSSYLA
VL CDR2 (SEQ ID NO: 801):
GQSSRTR
VL CDR3 (SEQ ID NO: 802):
QQFGSSQLFT

In one embodiment the bispecific antibody is bimAb05-5345 wherein the anti-FIX(a) arm comprises the following CDR-sequences:

VH CDR1 (SEQ ID NO: 52):
DYAMH
VH CDR2 (SEQ ID NO: 53):
GISWRGDIGGYVKSVKG
VH CDR3 (SEQ ID NO: 54):
SYGSGSFYNAFDS
VL CDR1 (SEQ ID NO: 56):
RASQSISSWLA
VL CDR2 (SEQ ID NO: 57):
KASKLER
VL CDR3 (SEQ ID NO: 58):
LEYSSYIRT

and wherein the anti-FX(a) arm comprises the following CDR-sequences:

VH CDR1 (SEQ ID NO: 820):
TSWIS
VH CDR2 (SEQ ID NO: 821):
MIDPSDSYTSYSPSFQG
VH CDR3 (SEQ ID NO: 822):
LHYYNSEEFDV
VL CDR1 (SEQ ID NO: 824):
RASQSVSSSYLA
VL CDR2 (SEQ ID NO: 825):
GQSSRTR
VL CDR3 (SEQ ID NO: 826):
QQFGESQLFT

In one embodiment the bispecific antibody is bimAb05-5346 wherein the anti-FIX(a) arm comprises the following CDR-sequences:

VH CDR1 (SEQ ID NO: 52):
DYAMH
VH CDR2 (SEQ ID NO: 53):
GISWRGDIGGYVKSVKG
VH CDR3 (SEQ ID NO: 54):
SYGSGSFYNAFDS
VL CDR1 (SEQ ID NO: 56):
RASQSISSWLA
VL CDR2 (SEQ ID NO: 57):
KASKLER
VL CDR3 (SEQ ID NO: 58):
LEYSSYIRT

and wherein the anti-FX(a) arm comprises the following CDR-sequences:

VH CDR1 (SEQ ID NO: 804):
TSWIV
VH CDR2 (SEQ ID NO: 805):
MIDPSDSYTSYSPSFQG
VH CDR3 (SEQ ID NO: 806):
LHYYNSEEFDV
VL CDR1 (SEQ ID NO: 808):
RASQSVSSSYLA
VL CDR2 (SEQ ID NO: 809):
GQSSRTR
VL CDR3 (SEQ ID NO: 810):
QQFGSSQLFT

In one embodiment the bispecific antibody is bimAb05-5347 wherein the anti-FIX(a) arm comprises the following CDR-sequences:

VH CDR1 (SEQ ID NO: 52):
DYAMH
VH CDR2 (SEQ ID NO: 53):
GISWRGDIGGYVKSVKG
VH CDR3 (SEQ ID NO: 54):
SYGSGSFYNAFDS
VL CDR1 (SEQ ID NO: 56):
RASQSISSWLA
VL CDR2 (SEQ ID NO: 57):
KASKLER
VL CDR3 (SEQ ID NO: 58):
LEYSSYIRT

and wherein the anti-FX(a) arm comprises the following CDR-sequences:

VH CDR1 (SEQ ID NO: 828):
TSWIS
VH CDR2 (SEQ ID NO: 829):
MIDPSDSFTSYSPSFQG
VH CDR3 (SEQ ID NO: 830):
LHYYHSEEFDV
VL CDR1 (SEQ ID NO: 832):
RASQSVSSSYLA
VL CDR2 (SEQ ID NO: 833):
GQSSRTR
VL CDR3 (SEQ ID NO: 834):
QQFGESQLFT

In one embodiment the bispecific antibody is bimAb05-5348 wherein the anti-FIX(a) arm comprises the following CDR-sequences:

VH CDR1 (SEQ ID NO: 52):
DYAMH
VH CDR2 (SEQ ID NO: 53):
GISWRGDIGGYVKSVKG
VH CDR3 (SEQ ID NO: 54):
SYGSGSFYNAFDS
VL CDR1 (SEQ ID NO: 56):
RASQSISSWLA
VL CDR2 (SEQ ID NO: 57):
KASKLER
VL CDR3 (SEQ ID NO: 58):
LEYSSYIRT

and wherein the anti-FX(a) arm comprises the following CDR-sequences:

VH CDR1 (SEQ ID NO: 812):
TSWIV
VH CDR2 (SEQ ID NO: 813):
MIDPSDSYTSYSPSFQG
VH CDR3 (SEQ ID NO: 814):
LHYYNSEEFDV
VL CDR1 (SEQ ID NO: 816):
RASQSVSSSYLA
VL CDR2 (SEQ ID NO: 817):
GQSSRTR
VL CDR3 (SEQ ID NO: 818):
QQFGSSQLFT

In one embodiment the bispecific antibody is bimAb05-5349 wherein the anti-FIX(a) arm comprises the following CDR-sequences:

VH CDR1 (SEQ ID NO: 52):
DYAMH
VH CDR2 (SEQ ID NO: 53):
GISWRGDIGGYVKSVKG
VH CDR3 (SEQ ID NO: 54):
SYGSGSFYNAFDS
VL CDR1 (SEQ ID NO: 56):
RASQSISSWLA
VL CDR2 (SEQ ID NO: 57):
KASKLER
VL CDR3 (SEQ ID NO: 58):
LEYSSYIRT

and wherein the anti-FX(a) arm comprises the following CDR-sequences:

VH CDR1 (SEQ ID NO: 836):
TSWIS
VH CDR2 (SEQ ID NO: 837):
MIDPSDSYTSYSPSFQG
VH CDR3 (SEQ ID NO: 838):
LHYYHSEEFDV
VL CDR1 (SEQ ID NO: 840):
RASQSVSSSYLA
VL CDR2 (SEQ ID NO: 841):
GQSSRTR
VL CDR3 (SEQ ID NO: 842):
QQFGESQLFT

In one embodiment the bispecific antibody is bimAb05-5350 wherein the anti-FIX(a) arm comprises the following CDR-sequences:

VH CDR1 (SEQ ID NO: 52):
DYAMH
VH CDR2 (SEQ ID NO: 53):
GISWRGDIGGYVKSVKG
VH CDR3 (SEQ ID NO: 54):
SYGSGSFYNAFDS
VL CDR1 (SEQ ID NO: 56):
RASQSISSWLA
VL CDR2 (SEQ ID NO: 57):
KASKLER
VL CDR3 (SEQ ID NO: 58):
LEYSSYIRT

and wherein the anti-FX(a) arm comprises the following CDR-sequences:

VH CDR1 (SEQ ID NO: 716):
TSWIV
VH CDR2 (SEQ ID NO: 717):
MIDPSDSFTSYSPSFQG
VH CDR3 (SEQ ID NO: 718):
LHYYNSEEFDV
VL CDR1 (SEQ ID NO: 720):
RASQSVSSSYLA
VL CDR2 (SEQ ID NO: 721):
GQSSRTR
VL CDR3 (SEQ ID NO: 722):
QQFGESQLFT

In one embodiment the bispecific antibody is bimAb05-5351 wherein the anti-FIX(a) arm comprises the following CDR-sequences:

VH CDR1 (SEQ ID NO: 52):
DYAMH
VH CDR2 (SEQ ID NO: 53):
GISWRGDIGGYVKSVKG
VH CDR3 (SEQ ID NO: 54):
SYGSGSFYNAFDS
VL CDR1 (SEQ ID NO: 56):
RASQSISSWLA
VL CDR2 (SEQ ID NO: 57):
KASKLER
VL CDR3 (SEQ ID NO: 58):
LEYSSYIRT

and wherein the anti-FX(a) arm comprises the following CDR-sequences:

VH CDR1 (SEQ ID NO: 844):
TSWIS
VH CDR2 (SEQ ID NO: 845):
MIDPSDSYTSYSPSFQG
VH CDR3 (SEQ ID NO: 846):
LHYYHSEEFDV
VL CDR1 (SEQ ID NO: 848):
RASQSVSSSYLA
VL CDR2 (SEQ ID NO: 849):
GQSSRTR
VL CDR3 (SEQ ID NO: 850):
QQFGESQLFT

In one embodiment the bispecific antibody is bimAb05-5352 wherein the anti-FIX(a) arm comprises the following CDR-sequences:

VH CDR1 (SEQ ID NO: 52):
DYAMH
VH CDR2 (SEQ ID NO: 53):
GISWRGDIGGYVKSVKG
VH CDR3 (SEQ ID NO: 54):
SYGSGSFYNAFDS
VL CDR1 (SEQ ID NO: 56):
RASQSISSWLA
VL CDR2 (SEQ ID NO: 57):
KASKLER
VL CDR3 (SEQ ID NO: 58):
LEYSSYIRT

and wherein the anti-FX(a) arm comprises the following CDR-sequences:

VH CDR1 (SEQ ID NO: 852):
TSWIS
VH CDR2 (SEQ ID NO: 853):
MIDPSDSFTSYSPSFQG
VH CDR3 (SEQ ID NO: 854):
LHYYNSEEFDV
VL CDR1 (SEQ ID NO: 856):
RASQSVSSSYLA
VL CDR2 (SEQ ID NO: 857):
GQSSRTR
VL CDR3 (SEQ ID NO: 858):
QQFGESQLFT

In one embodiment the bispecific antibody is bimAb05-5353 wherein the anti-FIX(a) arm comprises the following CDR-sequences:

VH CDR1 (SEQ ID NO: 52):
DYAMH
VH CDR2 (SEQ ID NO: 53):
GISWRGDIGGYVKSVKG
VH CDR3 (SEQ ID NO: 54):
SYGSGSFYNAFDS
VL CDR1 (SEQ ID NO: 56):
RASQSISSWLA
VL CDR2 (SEQ ID NO: 57):
KASKLER
VL CDR3 (SEQ ID NO: 58):
LEYSSYIRT

and wherein the anti-FX(a) arm comprises the following CDR-sequences:

VH CDR1 (SEQ ID NO: 876):
TSWIS
VH CDR2 (SEQ ID NO: 877):
MIDPSDSYTSYSPSFQG
VH CDR3 (SEQ ID NO: 878):
LHYYNSEEFDV
VL CDR1 (SEQ ID NO: 880):
RASQSVSSSYLA
VL CDR2 (SEQ ID NO: 881):
GQSSRTR
VL CDR3 (SEQ ID NO: 882):
QQFGNSQLFT

In one embodiment the bispecific antibody is bimAb05-5354 wherein the anti-FIX(a) arm comprises the following CDR-sequences:

VH CDR1 (SEQ ID NO: 52):
DYAMH
VH CDR2 (SEQ ID NO: 53):
GISWRGDIGGYVKSVKG
VH CDR3 (SEQ ID NO: 54):
SYGSGSFYNAFDS
VL CDR1 (SEQ ID NO: 56):
RASQSISSWLA
VL CDR2 (SEQ ID NO: 57):
KASKLER
VL CDR3 (SEQ ID NO: 58):
LEYSSYIRT

and wherein the anti-FX(a) arm comprises the following CDR-sequences:

VH CDR1 (SEQ ID NO: 860):
TSWIV
VH CDR2 (SEQ ID NO: 861):
MIDPSDSYTSYSPSFQG
VH CDR3 (SEQ ID NO: 862):
LHYYNSEEFDV
VL CDR1 (SEQ ID NO: 864):
RASQSVSSSYLA
VL CDR2 (SEQ ID NO: 865):
GQSSRTR
VL CDR3 (SEQ ID NO: 866):
QQFGESQLFT

In one embodiment the bispecific antibody is bimAb05-5355 wherein the anti-FIX(a) arm comprises the following CDR-sequences:

VH CDR1 (SEQ ID NO: 52):
DYAMH
VH CDR2 (SEQ ID NO: 53):
GISWRGDIGGYVKSVKG
VH CDR3 (SEQ ID NO: 54):
SYGSGSFYNAFDS
VL CDR1 (SEQ ID NO: 56):
RASQSISSWLA
VL CDR2 (SEQ ID NO: 57):
KASKLER
VL CDR3 (SEQ ID NO: 58):
LEYSSYIRT

and wherein the anti-FX(a) arm comprises the following CDR-sequences:

VH CDR1 (SEQ ID NO: 884):
TSWIS
VH CDR2 (SEQ ID NO: 885):
MIDPSDSFTSYSPSFQG
VH CDR3 (SEQ ID NO: 886):
LHYYHSEEFDV
VL CDR1 (SEQ ID NO: 888):
RASQSVSSSYLA
VL CDR2 (SEQ ID NO: 889):
GQSSRTR
VL CDR3 (SEQ ID NO: 890):
QQFGNSQLFT

In one embodiment the bispecific antibody is bimAb05-5356 wherein the anti-FIX(a) arm comprises the following CDR-sequences:

VH CDR1 (SEQ ID NO: 52):
DYAMH
VH CDR2 (SEQ ID NO: 53):
GISWRGDIGGYVKSVKG
VH CDR3 (SEQ ID NO: 54):
SYGSGSFYNAFDS
VL CDR1 (SEQ ID NO: 56):
RASQSISSWLA
VL CDR2 (SEQ ID NO: 57):
KASKLER
VL CDR3 (SEQ ID NO: 58):
LEYSSYIRT

and wherein the anti-FX(a) arm comprises the following CDR-sequences:

VH CDR1 (SEQ ID NO: 868):
TSWIV
VH CDR2 (SEQ ID NO: 869):
MIDPSDSYTSYSPSFQG
VH CDR3 (SEQ ID NO: 870):
LHYYNSEEFDV
VL CDR1 (SEQ ID NO: 872):
RASQSVSSSYLA
VL CDR2 (SEQ ID NO: 873):
GQSSRTR
VL CDR3 (SEQ ID NO: 874):
QQFGESQLFT

In one embodiment the bispecific antibody is bimAb05-5357 wherein the anti-FIX(a) arm comprises the following CDR-sequences:

VH CDR1 (SEQ ID NO: 52):
DYAMH
VH CDR2 (SEQ ID NO: 53):
GISWRGDIGGYVKSVKG
VH CDR3 (SEQ ID NO: 54):
SYGSGSFYNAFDS
VL CDR1 (SEQ ID NO: 56):
RASQSISSWLA
VL CDR2 (SEQ ID NO: 57):
KASKLER
VL CDR3 (SEQ ID NO: 58):
LEYSSYIRT

and wherein the anti-FX(a) arm comprises the following CDR-sequences:

VH CDR1 (SEQ ID NO: 892):
TSWIS
VH CDR2 (SEQ ID NO: 893):
MIDPSDSYTSYSPSFQG
VH CDR3 (SEQ ID NO: 894):
LHYYHSEEFDV
VL CDR1 (SEQ ID NO: 896):
RASQSVSSSYLA
VL CDR2 (SEQ ID NO: 897):
GQSSRTR
VL CDR3 (SEQ ID NO: 898):
QQFGNSQLFT

In one embodiment the bispecific antibody is bimAb05-5358 wherein the anti-FIX(a) arm comprises the following CDR-sequences:

VH CDR1 (SEQ ID NO: 52):
DYAMH
VH CDR2 (SEQ ID NO: 53):
GISWRGDIGGYVKSVKG
VH CDR3 (SEQ ID NO: 54):
SYGSGSFYNAFDS
VL CDR1 (SEQ ID NO: 56):
RASQSISSWLA
VL CDR2 (SEQ ID NO: 57):
KASKLER
VL CDR3 (SEQ ID NO: 58):
LEYSSYIRT

and wherein the anti-FX(a) arm comprises the following CDR-sequences:

VH CDR1 (SEQ ID NO: 724):
TSWIV
VH CDR2 (SEQ ID NO: 725):
MIDPSDSFTSYSPSFQG
VH CDR3 (SEQ ID NO: 726):
LHYYNSEEFDV
VL CDR1 (SEQ ID NO: 728):
RASQSVSSSYLA
VL CDR2 (SEQ ID NO: 729):
GQSSRTR
VL CDR3 (SEQ ID NO: 730):
QQFGNSQLFT

In one embodiment the bispecific antibody is bimAb05-5359 wherein the anti-FIX(a) arm comprises the following CDR-sequences:

VH CDR1 (SEQ ID NO: 52): DYAMH
VH CDR2 (SEQ ID NO: 53): GISWRGDIGGYVKSVKG
VH CDR3 (SEQ ID NO: 54): SYGSGSFYNAFDS
VL CDR1 (SEQ ID NO: 56): RASQSISSWLA
VL CDR2 (SEQ ID NO: 57): KASKLER
VL CDR3 (SEQ ID NO: 58): LEYSSYIRT

and wherein the anti-FX(a) arm comprises the following CDR-sequences:

VH CDR1 (SEQ ID NO: 900): TSWIS
VH CDR2 (SEQ ID NO: 901): MIDPSDSYTSYSPSFQG
VH CDR3 (SEQ ID NO: 902): LHYYHSEEFDV
VL CDR1 (SEQ ID NO: 904): RASQSVSSSYLA
VL CDR2 (SEQ ID NO: 905): GQSSRTR
VL CDR3 (SEQ ID NO: 906): QQFGNSQLFT

In one embodiment the bispecific antibody is bimAb05-5361 wherein the anti-FIX(a) arm comprises the following CDR-sequences:

VH CDR1 (SEQ ID NO: 52): DYAMH
VH CDR2 (SEQ ID NO: 53): GISWRGDIGGYVKSVKG
VH CDR3 (SEQ ID NO: 54): SYGSGSFYNAFDS
VL CDR1 (SEQ ID NO: 56): RASQSISSWLA
VL CDR2 (SEQ ID NO: 57): KASKLER
VL CDR3 (SEQ ID NO: 58): LEYSSYIRT

and wherein the anti-FX(a) arm comprises the following CDR-sequences:

VH CDR1 (SEQ ID NO: 924): TSWIV
VH CDR2 (SEQ ID NO: 925): MIDPSDSYTSYSPSFQG
VH CDR3 (SEQ ID NO: 926): LHYYNSEEFDV
VL CDR1 (SEQ ID NO: 928): RASQSVSSSYLA
VL CDR2 (SEQ ID NO: 929): GQSSRTR
VL CDR (SEQ ID NO: 930): QQFGNSQLFT

In one embodiment the bispecific antibody is bimAb05-5362 wherein the anti-FIX(a) arm comprises the following CDR-sequences:

VH CDR1 (SEQ ID NO: 52): DYAMH
VH CDR2 (SEQ ID NO: 53): GISWRGDIGGYVKSVKG
VH CDR3 (SEQ ID NO: 54): SYGSGSFYNAFDS
VL CDR1 (SEQ ID NO: 56): RASQSISSWLA
VL CDR2 (SEQ ID NO: 57): KASKLER
VL CDR3 (SEQ ID NO: 58): LEYSSYIRT

and wherein the anti-FX(a) arm comprises the following CDR-sequences:

VH CDR1 (SEQ ID NO: 908): TSWIS
VH CDR2 (SEQ ID NO: 909): MIDPSDSFTSYSPSFQG
VH CDR3 (SEQ ID NO: 910): LHYYNSEEFDV
VL CDR1 (SEQ ID NO: 912): RASQSVSSSYLA
VL CDR2 (SEQ ID NO: 913): GQSSRTR
VL CDR3 (SEQ ID NO: 914): QQFGNSQLFT

In one embodiment the bispecific antibody is bimAb05-5363 wherein the anti-FIX(a) arm comprises the following CDR-sequences:

VH CDR1 (SEQ ID NO: 52): DYAMH
VH CDR2 (SEQ ID NO: 53): GISWRGDIGGYVKSVKG
VH CDR3 (SEQ ID NO: 54): SYGSGSFYNAFDS
VL CDR1 (SEQ ID NO: 56): RASQSISSWLA
VL CDR2 (SEQ ID NO: 57): KASKLER
VL CDR3 (SEQ ID NO: 58): LEYSSYIRT

and wherein the anti-FX(a) arm comprises the following CDR-sequences:

VH CDR1 (SEQ ID NO: 732): TSWIV
VH CDR2 (SEQ ID NO: 733): MIDPSDSFTSYSPSFQG
VH CDR3 (SEQ ID NO: 734): LHYYNSEEFDV
VL CDR1 (SEQ ID NO: 736): RASQSVSSSYLA
VL CDR2 (SEQ ID NO: 737): GQSSRTR
VL CDR3 (SEQ ID NO: 738): QQFGQSQLFT

In one embodiment the bispecific antibody is bimAb05-5364 wherein the anti-FIX(a) arm comprises the following CDR-sequences:

VH CDR1 (SEQ ID NO: 52): DYAMH
VH CDR2 (SEQ ID NO: 53): GISWRGDIGGYVKSVKG
VH CDR3 (SEQ ID NO: 54): SYGSGSFYNAFDS
VL CDR1 (SEQ ID NO: 56): RASQSISSWLA
VL CDR2 (SEQ ID NO: 57): KASKLER
VL CDR3 (SEQ ID NO: 58): LEYSSYIRT

and wherein the anti-FX(a) arm comprises the following CDR-sequences:

VH CDR1 (SEQ ID NO: 916): TSWIV
VH CDR2 (SEQ ID NO: 917): MIDPSDSYTSYSPSFQG
VH CDR3 (SEQ ID NO: 918): LHYYNSEEFDV
VL CDR1 (SEQ ID NO: 920): RASQSVSSSYLA
VL CDR2 (SEQ ID NO: 921): GQSSRTR
VL CDR3 (SEQ ID NO: 922): QQFGNSQLFT

In one embodiment the bispecific antibody is bimAb05-5365 wherein the anti-FIX(a) arm comprises the following CDR-sequences:

VH CDR1 (SEQ ID NO: 52): DYAMH
VH CDR2 (SEQ ID NO: 53): GISWRGDIGGYVKSVKG
VH CDR3 (SEQ ID NO: 54): SYGSGSFYNAFDS
VL CDR1 (SEQ ID NO: 56): RASQSISSWLA
VL CDR2 (SEQ ID NO: 57): KASKLER
VL CDR3 (SEQ ID NO: 58): LEYSSYIRT

and wherein the anti-FX(a) arm comprises the following CDR-sequences:

VH CDR1 (SEQ ID NO: 932): TSWIS
VH CDR2 (SEQ ID NO: 933): MIDPSDSYTSYSPSFQG
VH CDR3 (SEQ ID NO: 934): LHYYNSEEFDV
VL CDR1 (SEQ ID NO: 936): RASQSVSSSYLA
VL CDR2 (SEQ ID NO: 937): GQSSRTR
VL CDR3 (SEQ ID NO: 938): QQFGQSQLFT

In one embodiment the bispecific antibody is bimAb05-5366 wherein the anti-FIX(a) arm comprises the following CDR-sequences:

VH CDR1 (SEQ ID NO: 52): DYAMH
VH CDR2 (SEQ ID NO: 53): GISWRGDIGGYVKSVKG
VH CDR3 (SEQ ID NO: 54): SYGSGSFYNAFDS
VL CDR1 (SEQ ID NO: 56): RASQSISSWLA
VL CDR2 (SEQ ID NO: 57): KASKLER
VL CDR3 (SEQ ID NO: 58): LEYSSYIRT

and wherein the anti-FX(a) arm comprises the following CDR-sequences:

VH CDR1 (SEQ ID NO: 940): TSWIS
VH CDR2 (SEQ ID NO: 941): MIDPSDSFTSYSPSFQG
VH CDR3 (SEQ ID NO: 942): LHYYHSEEFDV
VL CDR1 (SEQ ID NO: 944): RASQSVSSSYLA
VL CDR2 (SEQ ID NO: 945): GQSSRTR
VL CDR3 (SEQ ID NO: 946): QQFGQSQLFT

In one embodiment the bispecific antibody is bimAb05-5367 wherein the anti-FIX(a) arm comprises the following CDR-sequences:

VH CDR1 (SEQ ID NO: 52): DYAMH
VH CDR2 (SEQ ID NO: 53): GISWRGDIGGYVKSVKG
VH CDR3 (SEQ ID NO: 54): SYGSGSFYNAFDS
VL CDR1 (SEQ ID NO: 56): RASQSISSWLA
VL CDR2 (SEQ ID NO: 57): KASKLER
VL CDR3 (SEQ ID NO: 58): LEYSSYIRT

and wherein the anti-FX(a) arm comprises the following CDR-sequences:

VH CDR1 (SEQ ID NO: 964):
TSWIV
VH CDR2 (SEQ ID NO: 965):
MIDPSDSYTSYSPSFQG
VH CDR3 (SEQ ID NO: 966):
LHYYNSEEFDV
VL CDR1 (SEQ ID NO: 968):
RASQSVSSSYLA
VL CDR2 (SEQ ID NO: 969):
GQSSRTR
VL CDR3 (SEQ ID NO: 970):
QQFGQSQLFT

In one embodiment the bispecific antibody is bimAb05-5369 wherein the anti-FIX(a) arm comprises the following CDR-sequences:

VH CDR1 (SEQ ID NO: 52):
DYAMH
VH CDR2 (SEQ ID NO: 53):
GISWRGDIGGYVKSVKG
VH CDR3 (SEQ ID NO: 54):
SYGSGSFYNAFDS
VL CDR1 (SEQ ID NO: 56):
RASQSISSWLA
VL CDR2 (SEQ ID NO: 57):
KASKLER
VL CDR3 (SEQ ID NO: 58):
LEYSSYIRT

and wherein the anti-FX(a) arm comprises the following CDR-sequences:

VH CDR1 (SEQ ID NO: 972):
TSWIV
VH CDR2 (SEQ ID NO: 973):
MIDPSDSYTSYSPSFQG
VH CDR3 (SEQ ID NO: 974):
LHYYNSEEFDV
VL CDR1 (SEQ ID NO: 976):
RASQSVSSSYLA
VL CDR2 (SEQ ID NO: 977):
GQSSRTR
VL CDR3 (SEQ ID NO: 978):
QQFGQSQLFT

In one embodiment the bispecific antibody is bimAb05-5370 wherein the anti-FIX(a) arm comprises the following CDR-sequences:

VH CDR1 (SEQ ID NO: 52):
DYAMH
VH CDR2 (SEQ ID NO: 53):
GISWRGDIGGYVKSVKG
VH CDR3 (SEQ ID NO: 54):
SYGSGSFYNAFDS
VL CDR1 (SEQ ID NO: 56):
RASQSISSWLA
VL CDR2 (SEQ ID NO: 57):
KASKLER
VL CDR3 (SEQ ID NO: 58):
LEYSSYIRT

and wherein the anti-FX(a) arm comprises the following CDR-sequences:

VH CDR1 (SEQ ID NO: 948):
TSWIS
VH CDR2 (SEQ ID NO: 949):
MIDPSDSYTSYSPSFQG
VH CDR3 (SEQ ID NO: 950):
LHYYHSEEFDV
VL CDR1 (SEQ ID NO: 952):
RASQSVSSSYLA
VL CDR2 (SEQ ID NO: 953):
GQSSRTR
VL CDR3 (SEQ ID NO: 954):
QQFGQSQLFT

In one embodiment the bispecific antibody is bimAb05-5371 wherein the anti-FIX(a) arm comprises the following CDR-sequences:

VH CDR1 (SEQ ID NO: 52):
DYAMH
VH CDR2 (SEQ ID NO: 53):
GISWRGDIGGYVKSVKG
VH CDR3 (SEQ ID NO: 54):
SYGSGSFYNAFDS
VL CDR1 (SEQ ID NO: 56):
RASQSISSWLA
VL CDR2 (SEQ ID NO: 57):
KASKLER
VL CDR3 (SEQ ID NO: 58):
LEYSSYIRT

and wherein the anti-FX(a) arm comprises the following CDR-sequences:

VH CDR1 (SEQ ID NO: 740):
TSWIV
VH CDR2 (SEQ ID NO: 741):
MIDPSDSFTSYSPSFQG
VH CDR3 (SEQ ID NO: 742):
LHYYNSEEFDV
VL CDR1 (SEQ ID NO: 744):
RASQSVSSSYLA
VL CDR2 (SEQ ID NO: 745):
GQSSRTR
VL CDR3 (SEQ ID NO: 746):
QQFGDAQLFT

In one embodiment the bispecific antibody is bimAb05-5372 wherein the anti-FIX(a) arm comprises the following CDR-sequences:

VH CDR1 (SEQ ID NO: 52):
DYAMH
VH CDR2 (SEQ ID NO: 53):
GISWRGDIGGYVKSVKG
VH CDR3 (SEQ ID NO: 54):
SYGSGSFYNAFDS
VL CDR1 (SEQ ID NO: 56):
RASQSISSWLA
VL CDR2 (SEQ ID NO: 57):
KASKLER
VL CDR3 (SEQ ID NO: 58):
LEYSSYIRT

and wherein the anti-FX(a) arm comprises the following CDR-sequences:

VH CDR1 (SEQ ID NO: 956):
TSWIS
VH CDR2 (SEQ ID NO: 957):
MIDPSDSFTSYSPSFQG
VH CDR3 (SEQ ID NO: 958):
LHYYNSEEFDV
VL CDR1 (SEQ ID NO: 960):
RASQSVSSSYLA
VL CDR2 (SEQ ID NO: 961):
GQSSRTR
VL CDR3 (SEQ ID NO: 962):
QQFGQSQLFT

In one embodiment the bispecific antibody is bimAb05-5373 wherein the anti-FIX(a) arm comprises the following CDR-sequences:

VH CDR1 (SEQ ID NO: 52):
DYAMH
VH CDR2 (SEQ ID NO: 53):
GISWRGDIGGYVKSVKG
VH CDR3 (SEQ ID NO: 54):
SYGSGSFYNAFDS
VL CDR1 (SEQ ID NO: 56):
RASQSISSWLA
VL CDR2 (SEQ ID NO: 57):
KASKLER
VL CDR3 (SEQ ID NO: 58):
LEYSSYIRT

and wherein the anti-FX(a) arm comprises the following CDR-sequences:

VH CDR1 (SEQ ID NO: 980):
TSWIS
VH CDR2 (SEQ ID NO: 981):
MIDPSDSYTSYSPSFQG
VH CDR3 (SEQ ID NO: 982):
LHYYNSEEFDV
VL CDR1 (SEQ ID NO: 984):
RASQSVSSSYLA
VL CDR2 (SEQ ID NO: 985):
GQSSRTR
VL CDR3 (SEQ ID NO: 986):
QQFGDAQLFT

In one embodiment the bispecific antibody is bimAb05-5374 wherein the anti-FIX(a) arm comprises the following CDR-sequences:

VH CDR1 (SEQ ID NO: 52):
DYAMH
VH CDR2 (SEQ ID NO: 53):
GISWRGDIGGYVKSVKG
VH CDR3 (SEQ ID NO: 54):
SYGSGSFYNAFDS
VL CDR1 (SEQ ID NO: 56):
RASQSISSWLA
VL CDR2 (SEQ ID NO: 57):
KASKLER
VL CDR3 (SEQ ID NO: 58):
LEYSSYIRT

and wherein the anti-FX(a) arm comprises the following CDR-sequences:

VH CDR1 (SEQ ID NO: 988):
TSWIS
VH CDR2 (SEQ ID NO: 989):
MIDPSDSFTSYSPSFQG
VH CDR3 (SEQ ID NO: 990):
LHYYHSEEFDV
VL CDR1 (SEQ ID NO: 992):
RASQSVSSSYLA
VL CDR2 (SEQ ID NO: 993):
GQSSRTR
VL CDR3 (SEQ ID NO: 994):
QQFGDAQLFT

In one embodiment the bispecific antibody is bimAb05-5375 wherein the anti-FIX(a) arm comprises the following CDR-sequences:

VH CDR1 (SEQ ID NO: 52):
DYAMH
VH CDR2 (SEQ ID NO: 53):
GISWRGDIGGYVKSVKG
VH CDR3 (SEQ ID NO: 54):
SYGSGSFYNAFDS
VL CDR1 (SEQ ID NO: 56):
RASQSISSWLA
VL CDR2 (SEQ ID NO: 57):
KASKLER
VL CDR3 (SEQ ID NO: 58):
LEYSSYIRT

and wherein the anti-FX(a) arm comprises the following CDR-sequences:

VH CDR1 (SEQ ID NO: 1012):
TSWIV
VH CDR2 (SEQ ID NO: 1013):
MIDPSDSYTSYSPSFQG
VH CDR3 (SEQ ID NO: 1014):
LHYYNSEEFDV
VL CDR1 (SEQ ID NO: 1016):
RASQSVSSSYLA
VL CDR2 (SEQ ID NO: 1017):
GQSSRTR
VL CDR3 (SEQ ID NO: 1018):
QQFGDAQLFT

In one embodiment the bispecific antibody is bimAb05-5377 wherein the anti-FIX(a) arm comprises the following CDR-sequences:

VH CDR1 (SEQ ID NO: 52):
DYAMH
VH CDR2 (SEQ ID NO: 53):
GISWRGDIGGYVKSVKG
VH CDR3 (SEQ ID NO: 54):
SYGSGSFYNAFDS
VL CDR1 (SEQ ID NO: 56):
RASQSISSWLA
VL CDR2 (SEQ ID NO: 57):
KASKLER
VL CDR3 (SEQ ID NO: 58):
LEYSSYIRT

and wherein the anti-FX(a) arm comprises the following CDR-sequences:

VH CDR1 (SEQ ID NO: 1020):
TSWIV
VH CDR2 (SEQ ID NO: 1021):
MIDPSDSYTSYSPSFQG
VH CDR3 (SEQ ID NO: 1022):
LHYYNSEEFDV
VL CDR1 (SEQ ID NO: 1024):
RASQSVSSSYLA
VL CDR2 (SEQ ID NO: 1025):
GQSSRTR
VL CDR3 (SEQ ID NO: 1026):
QQFGDAQLFT

In one embodiment the bispecific antibody is bimAb05-5378 wherein the anti-FIX(a) arm comprises the following CDR-sequences:

VH CDR1 (SEQ ID NO: 52):
DYAMH
VH CDR2 (SEQ ID NO: 53):
GISWRGDIGGYVKSVKG
VH CDR3 (SEQ ID NO: 54):
SYGSGSFYNAFDS
VL CDR1 (SEQ ID NO: 56):
RASQSISSWLA
VL CDR2 (SEQ ID NO: 57):
KASKLER
VL CDR3 (SEQ ID NO: 58):
LEYSSYIRT

and wherein the anti-FX(a) arm comprises the following CDR-sequences:

VH CDR1 (SEQ ID NO: 996):
TSWIS
VH CDR2 (SEQ ID NO: 997):
MIDPSDSYTSYSPSFQG
VH CDR3 (SEQ ID NO: 998):
LHYYHSEEFDV
VL CDR1 (SEQ ID NO: 1000):
RASQSVSSSYLA
VL CDR2 (SEQ ID NO: 1001):
GQSSRTR
VL CDR3 (SEQ ID NO: 1002):
QQFGDAQLFT

In one embodiment the bispecific antibody is bimAb05-5379 wherein the anti-FIX(a) arm comprises the following CDR-sequences:

VH CDR1 (SEQ ID NO: 52):
DYAMH
VH CDR2 (SEQ ID NO: 53):
GISWRGDIGGYVKSVKG
VH CDR3 (SEQ ID NO: 54):
SYGSGSFYNAFDS
VL CDR1 (SEQ ID NO: 56):
RASQSISSWLA
VL CDR2 (SEQ ID NO: 57):
KASKLER
VL CDR3 (SEQ ID NO: 58):
LEYSSYIRT

and wherein the anti-FX(a) arm comprises the following CDR-sequences:

VH CDR1 (SEQ ID NO: 748):
TSWIV
VH CDR2 (SEQ ID NO: 749):
MIDPSDSFTSYSPSFQG
VH CDR3 (SEQ ID NO: 750):
LHYYNSEEFDV
VL CDR1 (SEQ ID NO: 752):
RASQSVSSSYLA
VL CDR2 (SEQ ID NO: 753):
GQSSRTR
VL CDR3 (SEQ ID NO: 754):
QQFGDTQLFT

In one embodiment the bispecific antibody is bimAb05-5380 wherein the anti-FIX(a) arm comprises the following CDR-sequences:

VH CDR1 (SEQ ID NO: 52):
DYAMH
VH CDR2 (SEQ ID NO: 53):
GISWRGDIGGYVKSVKG
VH CDR3 (SEQ ID NO: 54):
SYGSGSFYNAFDS
VL CDR1 (SEQ ID NO: 56):
RASQSISSWLA
VL CDR2 (SEQ ID NO: 57):
KASKLER
VL CDR3 (SEQ ID NO: 58):
LEYSSYIRT

and wherein the anti-FX(a) arm comprises the following CDR-sequences:

VH CDR1 (SEQ ID NO: 1004):
TSWIS
VH CDR2 (SEQ ID NO: 1005):
MIDPSDSFTSYSPSFQG
VH CDR3 (SEQ ID NO: 1006):
LHYYNSEEFDV
VL CDR1 (SEQ ID NO: 1008):
RASQSVSSSYLA
VL CDR2 (SEQ ID NO: 1009):
GQSSRTR
VL CDR3 (SEQ ID NO: 1010):
QQFGDAQLFT

In one embodiment the bispecific antibody is bimAb05-5381 wherein the anti-FIX(a) arm comprises the following CDR-sequences:

VH CDR1 (SEQ ID NO: 52):
DYAMH
VH CDR2 (SEQ ID NO: 53):
GISWRGDIGGYVKSVKG
VH CDR3 (SEQ ID NO: 54):
SYGSGSFYNAFDS
VL CDR1 (SEQ ID NO: 56):
RASQSISSWLA
VL CDR2 (SEQ ID NO: 57):
KASKLER
VL CDR3 (SEQ ID NO: 58):
LEYSSYIRT

and wherein the anti-FX(a) arm comprises the following CDR-sequences:

VH CDR1 (SEQ ID NO: 1028):
TSWIS
VH CDR2 (SEQ ID NO: 1029):
MIDPSDSYTSYSPSFQG
VH CDR3 (SEQ ID NO: 1030):
LHYYNSEEFDV
VL CDR1 (SEQ ID NO: 1032):
RASQSVSSSYLA
VL CDR2 (SEQ ID NO: 1033):
GQSSRTR
VL CDR3 (SEQ ID NO: 1034):
QQFGDTQLFT

In one embodiment the bispecific antibody is bimAb05-5383 wherein the anti-FIX(a) arm comprises the following CDR-sequences:

VH CDR1 (SEQ ID NO: 52):
DYAMH
VH CDR2 (SEQ ID NO: 53):
GISWRGDIGGYVKSVKG
VH CDR3 (SEQ ID NO: 54):
SYGSGSFYNAFDS
VL CDR1 (SEQ ID NO: 56):
RASQSISSWLA
VL CDR2 (SEQ ID NO: 57):
KASKLER
VL CDR3 (SEQ ID NO: 58):
LEYSSYIRT

and wherein the anti-FX(a) arm comprises the following CDR-sequences:

VH CDR1 (SEQ ID NO: 1052):
TSWIS
VH CDR2 (SEQ ID NO: 1053):
MIDPSDSYTSYSPSFQG
VH CDR3 (SEQ ID NO: 1054):
LHYYHSEEFDV
VL CDR1 (SEQ ID NO: 1056):
RASQSVSSSYLA
VL CDR2 (SEQ ID NO: 1057):
GQSSRTR
VL CDR3 (SEQ ID NO: 1058):
QQFGDTQLFT

In one embodiment the bispecific antibody is bimAb05-5384 wherein the anti-FIX(a) arm comprises the following CDR-sequences:

VH CDR1 (SEQ ID NO: 52):
DYAMH
VH CDR2 (SEQ ID NO: 53):
GISWRGDIGGYVKSVKG
VH CDR3 (SEQ ID NO: 54):
SYGSGSFYNAFDS
VL CDR1 (SEQ ID NO: 56):
RASQSISSWLA
VL CDR2 (SEQ ID NO: 57):
KASKLER
VL CDR3 (SEQ ID NO: 58):
LEYSSYIRT

and wherein the anti-FX(a) arm comprises the following CDR-sequences:

VH CDR1 (SEQ ID NO: 1036):
TSWIS
VH CDR2 (SEQ ID NO: 1037):
MIDPSDSFTSYSPSFQG
VH CDR3 (SEQ ID NO: 1038):
LHYYHSEEFDV
VL CDR1 (SEQ ID NO: 1040):
RASQSVSSSYLA
VL CDR2 (SEQ ID NO: 1041):
GQSSRTR
VL CDR3 (SEQ ID NO: 1042):
QQFGDTQLFT

In one embodiment the bispecific antibody is bimAb05-5385 wherein the anti-FIX(a) arm comprises the following CDR-sequences:

VH CDR1 (SEQ ID NO: 52):
DYAMH
VH CDR2 (SEQ ID NO: 53):
GISWRGDIGGYVKSVKG
VH CDR3 (SEQ ID NO: 54):
SYGSGSFYNAFDS
VL CDR1 (SEQ ID NO: 56):
RASQSISSWLA
VL CDR2 (SEQ ID NO: 57):
KASKLER
VL CDR3 (SEQ ID NO: 58):
LEYSSYIRT

and wherein the anti-FX(a) arm comprises the following CDR-sequences:

VH CDR1 (SEQ ID NO: 1060):
TSWIS
VH CDR2 (SEQ ID NO: 1061):
MIDPSDSFTSYSPSFQG
VH CDR3 (SEQ ID NO: 1062):
LHYYNSEEFDV
VL CDR1 (SEQ ID NO: 1064):
RASQSVSSSYLA
VL CDR2 (SEQ ID NO: 1065):
GQSSRTR
VL CDR3 (SEQ ID NO: 1066):
QQFGDTQLFT

In one embodiment the bispecific antibody is bimAb05-5386 wherein the anti-FIX(a) arm comprises the following CDR-sequences:

VH CDR1 (SEQ ID NO: 52):
DYAMH
VH CDR2 (SEQ ID NO: 53):
GISWRGDIGGYVKSVKG
VH CDR3 (SEQ ID NO: 54):
SYGSGSFYNAFDS
VL CDR1 (SEQ ID NO: 56):
RASQSISSWLA
VL CDR2 (SEQ ID NO: 57):
KASKLER
VL CDR3 (SEQ ID NO: 58):
LEYSSYIRT

and wherein the anti-FX(a) arm comprises the following CDR-sequences:

VH CDR1 (SEQ ID NO: 1044):
TSWIS
VH CDR2 (SEQ ID NO: 1045):
MIDPSDSYTSYSPSFQG
VH CDR3 (SEQ ID NO: 1046):
LHYYHSEEFDV
VL CDR1 (SEQ ID NO: 1048):
RASQSVSSSYLA
VL CDR2 (SEQ ID NO: 1049):
GQSSRTR
VL CDR3 (SEQ ID NO: 1050):
QQFGDTQLFT

In one embodiment the bispecific antibody is bimAb05-5387 wherein the anti-FIX(a) arm comprises the following CDR-sequences:

VH CDR1 (SEQ ID NO: 52):
DYAMH
VH CDR2 (SEQ ID NO: 53):
GISWRGDIGGYVKSVKG
VH CDR3 (SEQ ID NO: 54):
SYGSGSFYNAFDS
VL CDR1 (SEQ ID NO: 56):
RASQSISSWLA
VL CDR2 (SEQ ID NO: 57):
KASKLER
VL CDR3 (SEQ ID NO: 58):
LEYSSYIRT

and wherein the anti-FX(a) arm comprises the following CDR-sequences:

VH CDR1 (SEQ ID NO: 1068):
TSWIV
VH CDR2 (SEQ ID NO: 1069):
MIDPSDSYTSYSPSFQG
VH CDR3 (SEQ ID NO: 1070):
LHYYNSEEFDV
VL CDR1 (SEQ ID NO: 1072):
RASQSVSSSYLA
VL CDR2 (SEQ ID NO: 1073):
GQSSRTR
VL CDR3 (SEQ ID NO: 1074):
QQFGDTQLFT

In one embodiment the bispecific antibody is bimAb05-5388 wherein the anti-FIX(a) arm comprises the following CDR-sequences:

VH CDR1 (SEQ ID NO: 52):
DYAMH
VH CDR2 (SEQ ID NO: 53):
GISWRGDIGGYVKSVKG
VH CDR3 (SEQ ID NO: 54):
SYGSGSFYNAFDS
VL CDR1 (SEQ ID NO: 56):
RASQSISSWLA
VL CDR2 (SEQ ID NO: 57):
KASKLER
VL CDR3 (SEQ ID NO: 58):
LEYSSYIRT

and wherein the anti-FX(a) arm comprises the following CDR-sequences:

VH CDR1 (SEQ ID NO: 1076):
TSWIV
VH CDR2 (SEQ ID NO: 1077):
MIDPSDSYTSYSPSFQG
VH CDR3 (SEQ ID NO: 1078):
LHYYNSEEFDV
VL CDR1 (SEQ ID NO: 1080):
RASQSVSSSYLA
VL CDR2 (SEQ ID NO: 1081):
GQSSRTR
VL CDR3 (SEQ ID NO: 1082):
QQFGDTQLFT

In one embodiment the bispecific antibody is bimAb05-5389 wherein the anti-FIX(a) arm comprises the following CDR-sequences:

VH CDR1 (SEQ ID NO: 52):
DYAMH
VH CDR2 (SEQ ID NO: 53):
GISWRGDIGGYVKSVKG
VH CDR3 (SEQ ID NO: 54):
SYGSGSFYNAFDS
VL CDR1 (SEQ ID NO: 56):
RASQSISSWLA
VL CDR2 (SEQ ID NO: 57):
KASKLER
VL CDR3 (SEQ ID NO: 58):
LEYSSYIRT

and wherein the anti-FX(a) arm comprises the following CDR-sequences:

VH CDR1 (SEQ ID NO: 1100):
TSWIS
VH CDR2 (SEQ ID NO: 1101):
MIDPSDSYTSYSPSFQG
VH CDR3 (SEQ ID NO: 1102):
LHYYHSEEFDV
VL CDR1 (SEQ ID NO: 1104):
RASQSVSSSYLA
VL CDR2 (SEQ ID NO: 1105):
GQSSRTR
VL CDR3 (SEQ ID NO: 1106):
QQFGDNQLFT

In one embodiment the bispecific antibody is bimAb05-5390 wherein the anti-FIX(a) arm comprises the following CDR-sequences:

VH CDR1 (SEQ ID NO: 52):
DYAMH
VH CDR2 (SEQ ID NO: 53):
GISWRGDIGGYVKSVKG
VH CDR3 (SEQ ID NO: 54):
SYGSGSFYNAFDS
VL CDR1 (SEQ ID NO: 56):
RASQSISSWLA
VL CDR2 (SEQ ID NO: 57):
KASKLER
VL CDR3 (SEQ ID NO: 58):
LEYSSYIRT

and wherein the anti-FX(a) arm comprises the following CDR-sequences:

VH CDR1 (SEQ ID NO: 756):
TSWIV
VH CDR2 (SEQ ID NO: 757):
MIDPSDSFTSYSPSFQG
VH CDR3 (SEQ ID NO: 758):
LHYYNSEEFDV
VL CDR1 (SEQ ID NO: 760):
RASQSVSSSYLA
VL CDR2 (SEQ ID NO: 761):
GQSSRTR
VL CDR3 (SEQ ID NO: 762):
QQFGDNQLFT

In one embodiment the bispecific antibody is bimAb05-5391 wherein the anti-FIX(a) arm comprises the following CDR-sequences:

VH CDR1 (SEQ ID NO: 52):
DYAMH
VH CDR2 (SEQ ID NO: 53):
GISWRGDIGGYVKSVKG
VH CDR3 (SEQ ID NO: 54):
SYGSGSFYNAFDS
VL CDR1 (SEQ ID NO: 56):
RASQSISSWLA
VL CDR2 (SEQ ID NO: 57):
KASKLER
VL CDR3 (SEQ ID NO: 58):
LEYSSYIRT

and wherein the anti-FX(a) arm comprises the following CDR-sequences:

VH CDR1 (SEQ ID NO: 1108):
TSWIS
VH CDR2 (SEQ ID NO: 1109):
MIDPSDSYTSYSPSFQG
VH CDR3 (SEQ ID NO: 1110):
LHYYHSEEFDV
VL CDR1 (SEQ ID NO: 1112):
RASQSVSSSYLA
VL CDR2 (SEQ ID NO: 1113):
GQSSRTR
VL CDR3 (SEQ ID NO: 1114):
QQFGDNQLFT

In one embodiment the bispecific antibody is bimAb05-5392 wherein the anti-FIX(a) arm comprises the following CDR-sequences:

VH CDR1 (SEQ ID NO: 52):
DYAMH
VH CDR2 (SEQ ID NO: 53):
GISWRGDIGGYVKSVKG
VH CDR3 (SEQ ID NO: 54):
SYGSGSFYNAFDS
VL CDR1 (SEQ ID NO: 56):
RASQSISSWLA
VL CDR2 (SEQ ID NO: 57):
KASKLER
VL CDR3 (SEQ ID NO: 58):
LEYSSYIRT

and wherein the anti-FX(a) arm comprises the following CDR-sequences:

VH CDR1 (SEQ ID NO: 1084):
TSWIS
VH CDR2 (SEQ ID NO: 1085):
MIDPSDSYTSYSPSFQG
VH CDR3 (SEQ ID NO: 1086):
LHYYNSEEFDV
VL CDR1 (SEQ ID NO: 1088):
RASQSVSSSYLA
VL CDR2 (SEQ ID NO: 1089):
GQSSRTR
VL CDR3 (SEQ ID NO: 1090):
QQFGDNQLFT

In one embodiment the bispecific antibody is bimAb05-5393 wherein the anti-FIX(a) arm comprises the following CDR-sequences:

VH CDR1 (SEQ ID NO: 52):
DYAMH
VH CDR2 (SEQ ID NO: 53):
GISWRGDIGGYVKSVKG
VH CDR3 (SEQ ID NO: 54):
SYGSGSFYNAFDS
VL CDR1 (SEQ ID NO: 56):
RASQSISSWLA
VL CDR2 (SEQ ID NO: 57):
KASKLER
VL CDR3 (SEQ ID NO: 58):
LEYSSYIRT

and wherein the anti-FX(a) arm comprises the following CDR-sequences:

VH CDR1 (SEQ ID NO: 1116):
TSWIS
VH CDR2 (SEQ ID NO: 1117):
MIDPSDSFTSYSPSFQG
VH CDR3 (SEQ ID NO: 1118):
LHYYNSEEFDV
VL CDR1 (SEQ ID NO: 1120):
RASQSVSSSYLA
VL CDR2 (SEQ ID NO: 1121):
GQSSRTR
VL CDR3 (SEQ ID NO: 1122):
QQFGDNQLFT

In one embodiment the bispecific antibody is bimAb05-5394 wherein the anti-FIX(a) arm comprises the following CDR-sequences:

VH CDR1 (SEQ ID NO: 52):
DYAMH
VH CDR2 (SEQ ID NO: 53):
GISWRGDIGGYVKSVKG
VH CDR3 (SEQ ID NO: 54):
SYGSGSFYNAFDS
VL CDR1 (SEQ ID NO: 56):
RASQSISSWLA
VL CDR2 (SEQ ID NO: 57):
KASKLER
VL CDR3 (SEQ ID NO: 58):
LEYSSYIRT

and wherein the anti-FX(a) arm comprises the following CDR-sequences:

VH CDR1 (SEQ ID NO: 1092):
TSWIS
VH CDR2 (SEQ ID NO: 1093):
MIDPSDSFTSYSPSFQG
VH CDR3 (SEQ ID NO: 1094):
LHYYHSEEFDV
VL CDR1 (SEQ ID NO: 1096):
RASQSVSSSYLA
VL CDR2 (SEQ ID NO: 1097):
GQSSRTR
VL CDR3 (SEQ ID NO: 1098):
QQFGDNQLFT

In one embodiment the bispecific antibody is bimAb05-5395 wherein the anti-FIX(a) arm comprises the following CDR-sequences:

VH CDR1 (SEQ ID NO: 52):
DYAMH
VH CDR2 (SEQ ID NO: 53):
GISWRGDIGGYVKSVKG
VH CDR3 (SEQ ID NO: 54):
SYGSGSFYNAFDS
VL CDR1 (SEQ ID NO: 56):
RASQSISSWLA
VL CDR2 (SEQ ID NO: 57):
KASKLER
VL CDR3 (SEQ ID NO: 58):
LEYSSYIRT

and wherein the anti-FX(a) arm comprises the following CDR-sequences:

VH CDR1 (SEQ ID NO: 1124):
TSWIV
VH CDR2 (SEQ ID NO: 1125):
MIDPSDSYTSYSPSFQG
VH CDR3 (SEQ ID NO: 1126):
LHYYNSEEFDV
VL CDR1 (SEQ ID NO: 1128):
RASQSVSSSYLA
VL CDR2 (SEQ ID NO: 1129):
GQSSRTR
VL CDR3 (SEQ ID NO: 1130):
QQFGDNQLFT

In one embodiment the bispecific antibody is bimAb05-5396 wherein the anti-FIX(a) arm comprises the following CDR-sequences:

VH CDR1 (SEQ ID NO: 52):
DYAMH
VH CDR2 (SEQ ID NO: 53):
GISWRGDIGGYVKSVKG
VH CDR3 (SEQ ID NO: 54):
SYGSGSFYNAFDS
VL CDR1 (SEQ ID NO: 56):
RASQSISSWLA
VL CDR2 (SEQ ID NO: 57):
KASKLER
VL CDR3 (SEQ ID NO: 58):
LEYSSYIRT

and wherein the anti-FX(a) arm comprises the following CDR-sequences:

VH CDR1 (SEQ ID NO: 1132):
TSWIV
VH CDR2 (SEQ ID NO: 1133):
MIDPSDSYTSYSPSFQG
VH CDR3 (SEQ ID NO: 1134):
LHYYNSEEFDV
VL CDR1 (SEQ ID NO: 1136):
RASQSVSSSYLA
VL CDR2 (SEQ ID NO: 1137):
GQSSRTR
VL CDR3 (SEQ ID NO: 1138):
QQFGDNQLFT

In one embodiment the bispecific antibody is bimAb05-5397 wherein the anti-FIX(a) arm comprises the following CDR-sequences:

VH CDR1 (SEQ ID NO: 52):
DYAMH
VH CDR2 (SEQ ID NO: 53):
GISWRGDIGGYVKSVKG
VH CDR3 (SEQ ID NO: 54):
SYGSGSFYNAFDS
VL CDR1 (SEQ ID NO: 56):
RASQSISSWLA
VL CDR2 (SEQ ID NO: 57):
KASKLER
VL CDR3 (SEQ ID NO: 58):
LEYSSYIRT

and wherein the anti-FX(a) arm comprises the following CDR-sequences:

VH CDR1 (SEQ ID NO: 1156):
TSWIS
VH CDR2 (SEQ ID NO: 1157):
MIDPSDSYTSYSPSFQG
VH CDR3 (SEQ ID NO: 1158):
LHYYHSEEFDV
VL CDR1 (SEQ ID NO: 1160):
RASQSVSSSYLA
VL CDR2 (SEQ ID NO: 1161):
GQSSRTR
VL CDR3 (SEQ ID NO: 1162):
QQFGDDQLFT

In one embodiment the bispecific antibody is bimAb05-5399 wherein the anti-FIX(a) arm comprises the following CDR-sequences:

VH CDR1 (SEQ ID NO: 52):
DYAMH
VH CDR2 (SEQ ID NO: 53):
GISWRGDIGGYVKSVKG
VH CDR3 (SEQ ID NO: 54):
SYGSGSFYNAFDS
VL CDR1 (SEQ ID NO: 56):
RASQSISSWLA
VL CDR2 (SEQ ID NO: 57):
KASKLER
VL CDR3 (SEQ ID NO: 58):
LEYSSYIRT

and wherein the anti-FX(a) arm comprises the following CDR-sequences:

VH CDR1 (SEQ ID NO: 1164):
TSWIS
VH CDR2 (SEQ ID NO: 1165):
MIDPSDSYTSYSPSFQG
VH CDR3 (SEQ ID NO: 1166):
LHYYHSEEFDV
VL CDR1 (SEQ ID NO: 1168):
RASQSVSSSYLA
VL CDR2 (SEQ ID NO: 1169):
GQSSRTR
VL CDR3 (SEQ ID NO: 1170):
QQFGDDQLFT

In one embodiment the bispecific antibody is bimAb05-5400 wherein the anti-FIX(a) arm comprises the following CDR-sequences:

VH CDR1 (SEQ ID NO: 52):
DYAMH
VH CDR2 (SEQ ID NO: 53):
GISWRGDIGGYVKSVKG
VH CDR3 (SEQ ID NO: 54):
SYGSGSFYNAFDS
VL CDR1 (SEQ ID NO: 56):
RASQSISSWLA
VL CDR2 (SEQ ID NO: 57):
KASKLER
VL CDR3 (SEQ ID NO: 58):
LEYSSYIRT

and wherein the anti-FX(a) arm comprises the following CDR-sequences:

VH CDR1 (SEQ ID NO: 1140):
TSWIS
VH CDR2 (SEQ ID NO: 1141):
MIDPSDSYTSYSPSFQG
VH CDR3 (SEQ ID NO: 1142):
LHYYNSEEFDV
VL CDR1 (SEQ ID NO: 1144):
RASQSVSSSYLA
VL CDR2 (SEQ ID NO: 1145):
GQSSRTR
VL CDR3 (SEQ ID NO: 1146):
QQFGDDQLFT

In one embodiment the bispecific antibody is bimAb05-5401 wherein the anti-FIX(a) arm comprises the following CDR-sequences:

VH CDR1 (SEQ ID NO: 52):
DYAMH
VH CDR2 (SEQ ID NO: 53):
GISWRGDIGGYVKSVKG
VH CDR3 (SEQ ID NO: 54):
SYGSGSFYNAFDS
VL CDR1 (SEQ ID NO: 56):
RASQSISSWLA
VL CDR2 (SEQ ID NO: 57):
KASKLER
VL CDR3 (SEQ ID NO: 58):
LEYSSYIRT

and wherein the anti-FX(a) arm comprises the following CDR-sequences:

VH CDR1 (SEQ ID NO: 1172):
TSWIS
VH CDR2 (SEQ ID NO: 1173):
MIDPSDSFTSYSPSFQG
VH CDR3 (SEQ ID NO: 1174):
LHYYNSEEFDV
VL CDR1 (SEQ ID NO: 1176):
RASQSVSSSYLA
VL CDR2 (SEQ ID NO: 1177):
GQSSRTR
VL CDR3 (SEQ ID NO: 1178):
QQFGDDQLFT

In one embodiment the bispecific antibody is bimAb05-5402 wherein the anti-FIX(a) arm comprises the following CDR-sequences:

VH CDR1 (SEQ ID NO: 52):
DYAMH
VH CDR2 (SEQ ID NO: 53):
GISWRGDIGGYVKSVKG
VH CDR3 (SEQ ID NO: 54):
SYGSGSFYNAFDS
VL CDR1 (SEQ ID NO: 56):
RASQSISSWLA
VL CDR2 (SEQ ID NO: 57):
KASKLER
VL CDR3 (SEQ ID NO: 58):
LEYSSYIRT

and wherein the anti-FX(a) arm comprises the following CDR-sequences:

VH CDR1 (SEQ ID NO: 1148):
TSWIS
VH CDR2 (SEQ ID NO: 1149):
MIDPSDSFTSYSPSFQG
VH CDR3 (SEQ ID NO: 1150):
LHYYHSEEFDV
VL CDR1 (SEQ ID NO: 1152):
RASQSVSSSYLA
VL CDR2 (SEQ ID NO: 1153):
GQSSRTR
VL CDR3 (SEQ ID NO: 1154):
QQFGDDQLFT

In one embodiment the bispecific antibody is bimAb05-5403 wherein the anti-FIX(a) arm comprises the following CDR-sequences:

VH CDR1 (SEQ ID NO: 52):
DYAMH
VH CDR2 (SEQ ID NO: 53):
GISWRGDIGGYVKSVKG
VH CDR3 (SEQ ID NO: 54):
SYGSGSFYNAFDS
VL CDR1 (SEQ ID NO: 56):
RASQSISSWLA
VL CDR2 (SEQ ID NO: 57):
KASKLER
VL CDR3 (SEQ ID NO: 58):
LEYSSYIRT

and wherein the anti-FX(a) arm comprises the following CDR-sequences:

VH CDR1 (SEQ ID NO: 1180):
TSWIV
VH CDR2 (SEQ ID NO: 1181):
MIDPSDSYTSYSPSFQG
VH CDR3 (SEQ ID NO: 1182):
LHYYNSEEFDV
VL CDR1 (SEQ ID NO: 1184):
RASQSVSSSYLA
VL CDR2 (SEQ ID NO: 1185):
GQSSRTR
VL CDR3 (SEQ ID NO: 1186):
QQFGDDQLFT

In one embodiment the bispecific antibody is bimAb05-5406 wherein the anti-FIX(a) arm comprises the following CDR-sequences:

VH CDR1 (SEQ ID NO: 52):
DYAMH
VH CDR2 (SEQ ID NO: 53):
GISWRGDIGGYVKSVKG
VH CDR3 (SEQ ID NO: 54):
SYGSGSFYNAFDS
VL CDR1 (SEQ ID NO: 56):
RASQSISSWLA
VL CDR2 (SEQ ID NO: 57):
KASKLER
VL CDR3 (SEQ ID NO: 58):
LEYSSYIRT

and wherein the anti-FX(a) arm comprises the following CDR-sequences:

VH CDR1 (SEQ ID NO: 1188):
TSWIV
VH CDR2 (SEQ ID NO: 1189):
MIDPSDSYTSYSPSFQG
VH CDR3 (SEQ ID NO: 1190):
LHYYNSEEFDV
VL CDR1 (SEQ ID NO: 1192):
RASQSVSSSYLA
VL CDR2 (SEQ ID NO: 1193):
GQSSRTR
VL CDR3 (SEQ ID NO: 1194):
QQFGDDQLFT

In one embodiment the bispecific antibody is bimAb05-5413 wherein the anti-FIX(a) arm comprises the following CDR-sequences:

VH CDR1 (SEQ ID NO: 52):
DYAMH
VH CDR2 (SEQ ID NO: 53):
GISWRGDIGGYVKSVKG
VH CDR3 (SEQ ID NO: 54):
SYGSGSFYNAFDS
VL CDR1 (SEQ ID NO: 56):
RASQSISSWLA
VL CDR2 (SEQ ID NO: 57):
KASKLER
VL CDR3 (SEQ ID NO: 58):
LEYSSYIRT

and wherein the anti-FX(a) arm comprises the following CDR-sequences:

VH CDR1 (SEQ ID NO: 548):
TSWIV
VH CDR2 (SEQ ID NO: 549):
MIDPSDSYTSYSPSFQG
VH CDR3 (SEQ ID NO: 550):
LHYYNSEEFDV
VL CDR1 (SEQ ID NO: 552):
RASQSVSSSYLA
VL CDR2 (SEQ ID NO: 553):
GQSSRTR
VL CDR3 (SEQ ID NO: 554):
QQFGDSQLFT

In one embodiment the bispecific antibody is bimAb05-4271 wherein the anti-FIX(a) arm comprises the following CDR-sequences:

VH CDR1 (SEQ ID NO: 1203):
DYAMH
VH CDR2 (SEQ ID NO: 1204):
GISWKGDIGGYVDSVKG
VH CDR3 (SEQ ID NO: 1205):
SYGSGSFYNAFDS
VL CDR1 (SEQ ID NO: 1207):
RASQSISSWLA
VL CDR2 (SEQ ID NO: 1208):
KASKLDR
VL CDR3 (SEQ ID NO: 1209):
LEYSSYIRT

and wherein the anti-FX(a) arm comprises the following CDR-sequences:

VH CDR1 (SEQ ID NO: 468):
TSWIV
VH CDR2 (SEQ ID NO: 469):
MIDPSDSFTSYSPSFQG
VH CDR3 (SEQ ID NO: 470):
LHYYNSEEFDV
VL CDR1 (SEQ ID NO: 472):
RASQSVSSSYLA
VL CDR2 (SEQ ID NO: 473):
GQSSRTR
VL CDR3 (SEQ ID NO: 474):
QQFGDSQLFT

In one embodiment the bispecific antibody is bimAb05-4756 wherein the anti-FIX(a) arm comprises the following CDR-sequences:

VH CDR1 (SEQ ID NO: 1211):
DYAMH
VH CDR2 (SEQ ID NO: 1212):
GISWRGDIGGYVKSVKG
VH CDR3 (SEQ ID NO: 1213):
SYGSGSFYNAFDS
VL CDR1 (SEQ ID NO: 1215):
RASQSISSWLA
VL CDR2 (SEQ ID NO: 1216):
KASKLER
VL CDR3 (SEQ ID NO: 1217):
LEYSSYIRT

and wherein the anti-FX(a) arm comprises the following CDR-sequences:

VH CDR1 (SEQ ID NO: 468):
TSWIV
VH CDR2 (SEQ ID NO: 469):
MIDPSDSFTSYSPSFQG
VH CDR3 (SEQ ID NO: 470):
LHYYNSEEFDV
VL CDR1 (SEQ ID NO: 472):
RASQSVSSSYLA
VL CDR2 (SEQ ID NO: 473):
GQSSRTR
VL CDR3 (SEQ ID NO: 474):
QQFGDSQLFT

In one embodiment the bispecific antibody is bimAb05-0396 wherein the anti-FIX(a) arm comprises the following CDR-sequences:

VH CDR1 (SEQ ID NO: 1219):
DYAMH
VH CDR2 (SEQ ID NO: 1220):
GISWRGDIGGYAKSVKG
VH CDR3 (SEQ ID NO: 1221):
SYGSGSFYNAFDS
VL CDR1 (SEQ ID NO: 1223):
RASQSISSWLA
VL CDR2 (SEQ ID NO: 1224):
KASKLDR
VL CDR3 (SEQ ID NO: 1225):
LEYSSYIRT

and wherein the anti-FX(a) arm comprises the following CDR-sequences:

VH CDR1 (SEQ ID NO: 468):
TSWIV
VH CDR2 (SEQ ID NO: 469):
MIDPSDSFTSYSPSFQG
VH CDR3 (SEQ ID NO: 470):
LHYYNSEEFDV
VL CDR1 (SEQ ID NO: 472):
RASQSVSSSYLA
VL CDR2 (SEQ ID NO: 473):
GQSSRTR
VL CDR3 (SEQ ID NO: 4741:
QQFGDSQLFT

In one embodiment the bispecific antibody is bimAb05-0417 wherein the anti-FIX(a) arm comprises the following CDR-sequences:

VH CDR1 (SEQ ID NO: 1227):
DYAMH
VH CDR2 (SEQ ID NO: 1228):
GISWRGDIGGYVKSVKG
VH CDR3 (SEQ ID NO: 1229):
SYGSGSFYNAFDS
VL CDR1 (SEQ ID NO: 1231):
RASQSISSWLA
VL CDR2 (SEQ ID NO: 1232):
KASKLDR
VL CDR3 (SEQ ID NO: 1233):
LEYSSYIRT

and wherein the anti-FX(a) arm comprises the following CDR-sequences:

VH CDR1 (SEQ ID NO: 468):
TSWIV
VH CDR2 (SEQ ID NO: 469):
MIDPSDSFTSYSPSFQG
VH CDR3 (SEQ ID NO: 470):
LHYYNSEEFDV
VL CDR1 (SEQ ID NO: 472):
RASQSVSSSYLA
VL CDR2 (SEQ ID NO: 473):
GQSSRTR
VL CDR3 (SEQ ID NO: 474):
QQFGDSQLFT

In one embodiment the bispecific antibody is bimAb05-0438 wherein the anti-FIX(a) arm comprises the following CDR-sequences:

VH CDR1 (SEQ ID NO: 1235):
DYAMH
VH CDR2 (SEQ ID NO: 1236):
GISWRGDIGGYVKSVKG
VH CDR3 (SEQ ID NO: 1237):
SYGSGSFYNAFDS
VL CDR1 (SEQ ID NO: 1239):
RASQSISSWLA
VL CDR2 (SEQ ID NO: 1240):
KASKLDR
VL CDR3 (SEQ ID NO: 1241):
LEYSSYIRT

and wherein the anti-FX(a) arm comprises the following CDR-sequences:

VH CDR1 (SEQ ID NO: 468):
TSWIV
VH CDR2 (SEQ ID NO: 469):
MIDPSDSFTSYSPSFQG
VH CDR3 (SEQ ID NO: 470):
LHYYNSEEFDV
VL CDR1 (SEQ ID NO: 472):
RASQSVSSSYLA
VL CDR2 (SEQ ID NO: 473):
GQSSRTR
VL CDR3 (SEQ ID NO: 474):
QQFGDSQLFT

In one embodiment the bispecific antibody comprises anti-FIX(a) arm VH and VL domains corresponding to SEQ ID NO:67 and SEQ ID NO:71, respectively, and

anti-FX(a) arm VH and VL domains corresponding to SEQ ID NO:467 and SEQ ID NO:471, respectively.

In one embodiment the bispecific antibody comprises anti-FIX(a) arm VH and VL domains corresponding to SEQ ID NO:43 and SEQ ID NO:47, respectively, and

anti-FX(a) arm VH and VL domains corresponding to SEQ ID NO:467 and SEQ ID NO:471, respectively.

In one embodiment the bispecific antibody comprises anti-FIX(a) arm VH and VL domains corresponding to SEQ ID NO:3 and SEQ ID NO:7, respectively, and

anti-FX(a) arm VH and VL domains corresponding to SEQ ID NO:459 and SEQ ID NO:463, respectively.

In one embodiment the bispecific antibody comprises anti-FIX(a) arm VH and VL domains corresponding to SEQ ID NO:67 and SEQ ID NO:71, respectively, and

anti-FX(a) arm VH and VL domains corresponding to SEQ ID NO:483 and SEQ ID NO:487, respectively.

In one embodiment the bispecific antibody comprises anti-FIX(a) arm VH and VL domains corresponding to SEQ ID NO:35 and SEQ ID NO:39, respectively, and

anti-FX(a) arm VH and VL domains corresponding to SEQ ID NO:483 and SEQ ID NO:487, respectively.

In one embodiment the bispecific antibody comprises anti-FIX(a) arm VH and VL domains corresponding to SEQ ID NO:51 and SEQ ID NO:55, respectively, and

anti-FX(a) arm VH and VL domains corresponding to SEQ ID NO:483 and SEQ ID NO:487, respectively.

In one embodiment the bispecific antibody comprises anti-FIX(a) arm VH and VL domains corresponding to SEQ ID NO:43 and SEQ ID NO:47, respectively, and

anti-FX(a) arm VH and VL domains corresponding to SEQ ID NO:483 and SEQ ID NO:487, respectively.

In one embodiment the bispecific antibody comprises anti-FIX(a) arm VH and VL domains corresponding to SEQ ID NO:3 and SEQ ID NO:7, respectively, and

anti-FX(a) arm VH and VL domains corresponding to SEQ ID NO:467 and SEQ ID NO:471, respectively.

In one embodiment the bispecific antibody comprises anti-FIX(a) arm VH and VL domains corresponding to SEQ ID NO:3 and SEQ ID NO:7, respectively, and

anti-FX(a) arm VH and VL domains corresponding to SEQ ID NO:475 and SEQ ID NO:479, respectively.

In one embodiment the bispecific antibody comprises anti-FIX(a) arm VH and VL domains corresponding to SEQ ID NO:11 and SEQ ID NO:15, respectively, and

anti-FX(a) arm VH and VL domains corresponding to SEQ ID NO:459 and SEQ ID NO:463, respectively.

In one embodiment the bispecific antibody comprises anti-FIX(a) arm VH and VL domains corresponding to SEQ ID NO:35 and SEQ ID NO:39, respectively, and

anti-FX(a) arm VH and VL domains corresponding to SEQ ID NO:459 and SEQ ID NO:463, respectively.

In one embodiment the bispecific antibody comprises anti-FIX(a) arm VH and VL domains corresponding to SEQ ID NO:43 and SEQ ID NO:47, respectively, and

anti-FX(a) arm VH and VL domains corresponding to SEQ ID NO:459 and SEQ ID NO:463, respectively.

In one embodiment the bispecific antibody comprises anti-FIX(a) arm VH and VL domains corresponding to SEQ ID NO:51 and SEQ ID NO:55, respectively, and

anti-FX(a) arm VH and VL domains corresponding to SEQ ID NO:459 and SEQ ID NO:463, respectively.

In one embodiment the bispecific antibody comprises anti-FIX(a) arm VH and VL domains corresponding to SEQ ID NO:67 and SEQ ID NO:71, respectively, and

anti-FX(a) arm VH and VL domains corresponding to SEQ ID NO:459 and SEQ ID NO:463, respectively.

In one embodiment the bispecific antibody comprises anti-FIX(a) arm VH and VL domains corresponding to SEQ ID NO:35 and SEQ ID NO:39, respectively, and

anti-FX(a) arm VH and VL domains corresponding to SEQ ID NO:467 and SEQ ID NO:471, respectively.

In one embodiment the bispecific antibody comprises anti-FIX(a) arm VH and VL domains corresponding to SEQ ID NO:51 and SEQ ID NO:55, respectively, and

anti-FX(a) arm VH and VL domains corresponding to SEQ ID NO:467 and SEQ ID NO:471, respectively.

In one embodiment the bispecific antibody comprises anti-FIX(a) arm VH and VL domains corresponding to SEQ ID NO:59 and SEQ ID NO:63, respectively, and

anti-FX(a) arm VH and VL domains corresponding to SEQ ID NO:467 and SEQ ID NO:471, respectively.

In one embodiment the bispecific antibody comprises anti-FIX(a) arm VH and VL domains corresponding to SEQ ID NO:19 and SEQ ID NO:23, respectively, and

anti-FX(a) arm VH and VL domains corresponding to SEQ ID NO:467 and SEQ ID NO:471, respectively.

In one embodiment the bispecific antibody comprises anti-FIX(a) arm VH and VL domains corresponding to SEQ ID NO:27 and SEQ ID NO:31, respectively, and

anti-FX(a) arm VH and VL domains corresponding to SEQ ID NO:467 and SEQ ID NO:471, respectively.

In one embodiment the bispecific antibody comprises anti-FIX(a) arm VH and VL domains corresponding to SEQ ID NO:75 and SEQ ID NO:79, respectively, and

anti-FX(a) arm VH and VL domains corresponding to SEQ ID NO:467 and SEQ ID NO:471, respectively.

In one embodiment the bispecific antibody comprises anti-FIX(a) arm VH and VL domains corresponding to SEQ ID NO:83 and SEQ ID NO:87, respectively, and

anti-FX(a) arm VH and VL domains corresponding to SEQ ID NO:467 and SEQ ID NO:471, respectively.

In one embodiment the bispecific antibody comprises anti-FIX(a) arm VH and VL domains corresponding to SEQ ID NO:91 and SEQ ID NO:95, respectively, and

anti-FX(a) arm VH and VL domains corresponding to SEQ ID NO:467 and SEQ ID NO:471, respectively.

In one embodiment the bispecific antibody comprises anti-FIX(a) arm VH and VL domains corresponding to SEQ ID NO:99 and SEQ ID NO:103, respectively, and

anti-FX(a) arm VH and VL domains corresponding to SEQ ID NO:467 and SEQ ID NO:471, respectively.

In one embodiment the bispecific antibody comprises anti-FIX(a) arm VH and VL domains corresponding to SEQ ID NO:107 and SEQ ID NO:111, respectively, and

anti-FX(a) arm VH and VL domains corresponding to SEQ ID NO:467 and SEQ ID NO:471, respectively.

In one embodiment the bispecific antibody comprises anti-FIX(a) arm VH and VL domains corresponding to SEQ ID NO:115 and SEQ ID NO:119, respectively, and

anti-FX(a) arm VH and VL domains corresponding to SEQ ID NO:467 and SEQ ID NO:471, respectively.

In one embodiment the bispecific antibody comprises anti-FIX(a) arm VH and VL domains corresponding to SEQ ID NO:211 and SEQ ID NO:215, respectively, and

anti-FX(a) arm VH and VL domains corresponding to SEQ ID NO:467 and SEQ ID NO:471, respectively.

In one embodiment the bispecific antibody comprises anti-FIX(a) arm VH and VL domains corresponding to SEQ ID NO:219 and SEQ ID NO:223, respectively, and

anti-FX(a) arm VH and VL domains corresponding to SEQ ID NO:467 and SEQ ID NO:471, respectively.

In one embodiment the bispecific antibody comprises anti-FIX(a) arm VH and VL domains corresponding to SEQ ID NO:227 and SEQ ID NO:231, respectively, and

anti-FX(a) arm VH and VL domains corresponding to SEQ ID NO:467 and SEQ ID NO:471, respectively.

In one embodiment the bispecific antibody comprises anti-FIX(a) arm VH and VL domains corresponding to SEQ ID NO:235 and SEQ ID NO:239, respectively, and

anti-FX(a) arm VH and VL domains corresponding to SEQ ID NO:467 and SEQ ID NO:471, respectively.

In one embodiment the bispecific antibody comprises anti-FIX(a) arm VH and VL domains corresponding to SEQ ID NO:251 and SEQ ID NO:255, respectively, and

anti-FX(a) arm VH and VL domains corresponding to SEQ ID NO:467 and SEQ ID NO:471, respectively.

In one embodiment the bispecific antibody comprises anti-FIX(a) arm VH and VL domains corresponding to SEQ ID NO:243 and SEQ ID NO:247, respectively, and

anti-FX(a) arm VH and VL domains corresponding to SEQ ID NO:467 and SEQ ID NO:471, respectively.

In one embodiment the bispecific antibody comprises anti-FIX(a) arm VH and VL domains corresponding to SEQ ID NO:259 and SEQ ID NO:263, respectively, and

anti-FX(a) arm VH and VL domains corresponding to SEQ ID NO:467 and SEQ ID NO:471, respectively.

In one embodiment the bispecific antibody comprises anti-FIX(a) arm VH and VL domains corresponding to SEQ ID NO:267 and SEQ ID NO:271, respectively, and

anti-FX(a) arm VH and VL domains corresponding to SEQ ID NO:467 and SEQ ID NO:471, respectively.

In one embodiment the bispecific antibody comprises anti-FIX(a) arm VH and VL domains corresponding to SEQ ID NO:123 and SEQ ID NO:127, respectively, and

anti-FX(a) arm VH and VL domains corresponding to SEQ ID NO:467 and SEQ ID NO:471, respectively.

In one embodiment the bispecific antibody comprises anti-FIX(a) arm VH and VL domains corresponding to SEQ ID NO:275 and SEQ ID NO:279, respectively, and

anti-FX(a) arm VH and VL domains corresponding to SEQ ID NO:467 and SEQ ID NO:471, respectively.

In one embodiment the bispecific antibody comprises anti-FIX(a) arm VH and VL domains corresponding to SEQ ID NO:283 and SEQ ID NO:287, respectively, and

anti-FX(a) arm VH and VL domains corresponding to SEQ ID NO:467 and SEQ ID NO:471, respectively.

In one embodiment the bispecific antibody comprises anti-FIX(a) arm VH and VL domains corresponding to SEQ ID NO:299 and SEQ ID NO:303, respectively, and

anti-FX(a) arm VH and VL domains corresponding to SEQ ID NO:467 and SEQ ID NO:471, respectively.

In one embodiment the bispecific antibody comprises anti-FIX(a) arm VH and VL domains corresponding to SEQ ID NO:291 and SEQ ID NO:295, respectively, and

anti-FX(a) arm VH and VL domains corresponding to SEQ ID NO:467 and SEQ ID NO:471, respectively.

In one embodiment the bispecific antibody comprises anti-FIX(a) arm VH and VL domains corresponding to SEQ ID NO:187 and SEQ ID NO:191, respectively, and

anti-FX(a) arm VH and VL domains corresponding to SEQ ID NO:467 and SEQ ID NO:471, respectively.

In one embodiment the bispecific antibody comprises anti-FIX(a) arm VH and VL domains corresponding to SEQ ID NO:139 and SEQ ID NO:143, respectively, and

anti-FX(a) arm VH and VL domains corresponding to SEQ ID NO:467 and SEQ ID NO:471, respectively.

In one embodiment the bispecific antibody comprises anti-FIX(a) arm VH and VL domains corresponding to SEQ ID NO:203 and SEQ ID NO:207, respectively, and

anti-FX(a) arm VH and VL domains corresponding to SEQ ID NO:467 and SEQ ID NO:471, respectively.

In one embodiment the bispecific antibody comprises anti-FIX(a) arm VH and VL domains corresponding to SEQ ID NO:179 and SEQ ID NO:183, respectively, and

anti-FX(a) arm VH and VL domains corresponding to SEQ ID NO:467 and SEQ ID NO:471, respectively.

In one embodiment the bispecific antibody comprises anti-FIX(a) arm VH and VL domains corresponding to SEQ ID NO:195 and SEQ ID NO:199, respectively, and

anti-FX(a) arm VH and VL domains corresponding to SEQ ID NO:467 and SEQ ID NO:471, respectively.

In one embodiment the bispecific antibody comprises anti-FIX(a) arm VH and VL domains corresponding to SEQ ID NO:171 and SEQ ID NO:175, respectively, and

anti-FX(a) arm VH and VL domains corresponding to SEQ ID NO:467 and SEQ ID NO:471, respectively.

In one embodiment the bispecific antibody comprises anti-FIX(a) arm VH and VL domains corresponding to SEQ ID NO:163 and SEQ ID NO:167, respectively, and

anti-FX(a) arm VH and VL domains corresponding to SEQ ID NO:467 and SEQ ID NO:471, respectively.

In one embodiment the bispecific antibody comprises anti-FIX(a) arm VH and VL domains corresponding to SEQ ID NO:147 and SEQ ID NO:151, respectively, and

anti-FX(a) arm VH and VL domains corresponding to SEQ ID NO:467 and SEQ ID NO:471, respectively.

In one embodiment the bispecific antibody comprises anti-FIX(a) arm VH and VL domains corresponding to SEQ ID NO:155 and SEQ ID NO:159, respectively, and

anti-FX(a) arm VH and VL domains corresponding to SEQ ID NO:467 and SEQ ID NO:471, respectively.

In one embodiment the bispecific antibody comprises anti-FIX(a) arm VH and VL domains corresponding to SEQ ID NO:51 and SEQ ID NO:55, respectively, and

anti-FX(a) arm VH and VL domains corresponding to SEQ ID NO:499 and SEQ ID NO:503, respectively.

In one embodiment the bispecific antibody comprises anti-FIX(a) arm VH and VL domains corresponding to SEQ ID NO:51 and SEQ ID NO:55, respectively, and

anti-FX(a) arm VH and VL domains corresponding to SEQ ID NO:507 and SEQ ID NO:511, respectively.

In one embodiment the bispecific antibody comprises anti-FIX(a) arm VH and VL domains corresponding to SEQ ID NO:51 and SEQ ID NO:55, respectively, and

anti-FX(a) arm VH and VL domains corresponding to SEQ ID NO:515 and SEQ ID NO:519, respectively.

In one embodiment the bispecific antibody comprises anti-FIX(a) arm VH and VL domains corresponding to SEQ ID NO:51 and SEQ ID NO:55, respectively, and

anti-FX(a) arm VH and VL domains corresponding to SEQ ID NO:523 and SEQ ID NO:527, respectively.

In one embodiment the bispecific antibody comprises anti-FIX(a) arm VH and VL domains corresponding to SEQ ID NO:51 and SEQ ID NO:55, respectively, and

anti-FX(a) arm VH and VL domains corresponding to SEQ ID NO:531 and SEQ ID NO:535, respectively.

In one embodiment the bispecific antibody comprises anti-FIX(a) arm VH and VL domains corresponding to SEQ ID NO:51 and SEQ ID NO:55, respectively, and

anti-FX(a) arm VH and VL domains corresponding to SEQ ID NO:539 and SEQ ID NO:543, respectively.

In one embodiment the bispecific antibody comprises anti-FIX(a) arm VH and VL domains corresponding to SEQ ID NO:51 and SEQ ID NO:55, respectively, and

anti-FX(a) arm VH and VL domains corresponding to SEQ ID NO:555 and SEQ ID NO:559, respectively.

In one embodiment the bispecific antibody comprises anti-FIX(a) arm VH and VL domains corresponding to SEQ ID NO:51 and SEQ ID NO:55, respectively, and

anti-FX(a) arm VH and VL domains corresponding to SEQ ID NO:563 and SEQ ID NO:567, respectively.

In one embodiment the bispecific antibody comprises anti-FIX(a) arm VH and VL domains corresponding to SEQ ID NO:51 and SEQ ID NO:55, respectively, and

anti-FX(a) arm VH and VL domains corresponding to SEQ ID NO:571 and SEQ ID NO:575, respectively.

In one embodiment the bispecific antibody comprises anti-FIX(a) arm VH and VL domains corresponding to SEQ ID NO:51 and SEQ ID NO:55, respectively, and

anti-FX(a) arm VH and VL domains corresponding to SEQ ID NO:579 and SEQ ID NO:583, respectively.

In one embodiment the bispecific antibody comprises anti-FIX(a) arm VH and VL domains corresponding to SEQ ID NO:51 and SEQ ID NO:55, respectively, and

anti-FX(a) arm VH and VL domains corresponding to SEQ ID NO:491 and SEQ ID NO:495, respectively.

In one embodiment the bispecific antibody comprises anti-FIX(a) arm VH and VL domains corresponding to SEQ ID NO:51 and SEQ ID NO:55, respectively, and

anti-FX(a) arm VH and VL domains corresponding to SEQ ID NO:587 and SEQ ID NO:591, respectively.

In one embodiment the bispecific antibody comprises anti-FIX(a) arm VH and VL domains corresponding to SEQ ID NO:51 and SEQ ID NO:55, respectively, and

anti-FX(a) arm VH and VL domains corresponding to SEQ ID NO:595 and SEQ ID NO:599, respectively.

In one embodiment the bispecific antibody comprises anti-FIX(a) arm VH and VL domains corresponding to SEQ ID NO:51 and SEQ ID NO:55, respectively, and

anti-FX(a) arm VH and VL domains corresponding to SEQ ID NO:603 and SEQ ID NO:607, respectively.

In one embodiment the bispecific antibody comprises anti-FIX(a) arm VH and VL domains corresponding to SEQ ID NO:51 and SEQ ID NO:55, respectively, and

anti-FX(a) arm VH and VL domains corresponding to SEQ ID NO:611 and SEQ ID NO:615, respectively.

In one embodiment the bispecific antibody comprises anti-FIX(a) arm VH and VL domains corresponding to SEQ ID NO:51 and SEQ ID NO:55, respectively, and

anti-FX(a) arm VH and VL domains corresponding to SEQ ID NO:619 and SEQ ID NO:623, respectively.

In one embodiment the bispecific antibody comprises anti-FIX(a) arm VH and VL domains corresponding to SEQ ID NO:51 and SEQ ID NO:55, respectively, and

anti-FX(a) arm VH and VL domains corresponding to SEQ ID NO:627 and SEQ ID NO:631, respectively.

In one embodiment the bispecific antibody comprises anti-FIX(a) arm VH and VL domains corresponding to SEQ ID NO:51 and SEQ ID NO:55, respectively, and

anti-FX(a) arm VH and VL domains corresponding to SEQ ID NO:635 and SEQ ID NO:639, respectively.

In one embodiment the bispecific antibody comprises anti-FIX(a) arm VH and VL domains corresponding to SEQ ID NO:51 and SEQ ID NO:55, respectively, and

anti-FX(a) arm VH and VL domains corresponding to SEQ ID NO:643 and SEQ ID NO:647, respectively.

In one embodiment the bispecific antibody comprises anti-FIX(a) arm VH and VL domains corresponding to SEQ ID NO:51 and SEQ ID NO:55, respectively, and

anti-FX(a) arm VH and VL domains corresponding to SEQ ID NO:651 and SEQ ID NO:655, respectively.

In one embodiment the bispecific antibody comprises anti-FIX(a) arm VH and VL domains corresponding to SEQ ID NO:51 and SEQ ID NO:55, respectively, and

anti-FX(a) arm VH and VL domains corresponding to SEQ ID NO:659 and SEQ ID NO:663, respectively.

In one embodiment the bispecific antibody comprises anti-FIX(a) arm VH and VL domains corresponding to SEQ ID NO:51 and SEQ ID NO:55, respectively, and

anti-FX(a) arm VH and VL domains corresponding to SEQ ID NO:667 and SEQ ID NO:671, respectively.

In one embodiment the bispecific antibody comprises anti-FIX(a) arm VH and VL domains corresponding to SEQ ID NO:51 and SEQ ID NO:55, respectively, and

anti-FX(a) arm VH and VL domains corresponding to SEQ ID NO:675 and SEQ ID NO:679, respectively.

In one embodiment the bispecific antibody comprises anti-FIX(a) arm VH and VL domains corresponding to SEQ ID NO:51 and SEQ ID NO:55, respectively, and

anti-FX(a) arm VH and VL domains corresponding to SEQ ID NO:683 and SEQ ID NO:687, respectively.

In one embodiment the bispecific antibody comprises anti-FIX(a) arm VH and VL domains corresponding to SEQ ID NO:51 and SEQ ID NO:55, respectively, and

anti-FX(a) arm VH and VL domains corresponding to SEQ ID NO:691 and SEQ ID NO:695, respectively.

In one embodiment the bispecific antibody comprises anti-FIX(a) arm VH and VL domains corresponding to SEQ ID NO:51 and SEQ ID NO:55, respectively, and

anti-FX(a) arm VH and VL domains corresponding to SEQ ID NO:699 and SEQ ID NO:703, respectively.

In one embodiment the bispecific antibody comprises anti-FIX(a) arm VH and VL domains corresponding to SEQ ID NO:307 and SEQ ID NO:311, respectively, and

anti-FX(a) arm VH and VL domains corresponding to SEQ ID NO:467 and SEQ ID NO:471, respectively.

In one embodiment the bispecific antibody comprises anti-FIX(a) arm VH and VL domains corresponding to SEQ ID NO:315 and SEQ ID NO:319, respectively, and

anti-FX(a) arm VH and VL domains corresponding to SEQ ID NO:467 and SEQ ID NO:471, respectively.

In one embodiment the bispecific antibody comprises anti-FIX(a) arm VH and VL domains corresponding to SEQ ID NO:339 and SEQ ID NO:343, respectively, and

anti-FX(a) arm VH and VL domains corresponding to SEQ ID NO:467 and SEQ ID NO:471, respectively.

In one embodiment the bispecific antibody comprises anti-FIX(a) arm VH and VL domains corresponding to SEQ ID NO:323 and SEQ ID NO:327, respectively, and

anti-FX(a) arm VH and VL domains corresponding to SEQ ID NO:467 and SEQ ID NO:471, respectively.

In one embodiment the bispecific antibody comprises anti-FIX(a) arm VH and VL domains corresponding to SEQ ID NO:331 and SEQ ID NO:335, respectively, and

anti-FX(a) arm VH and VL domains corresponding to SEQ ID NO:467 and SEQ ID NO:471, respectively.

In one embodiment the bispecific antibody comprises anti-FIX(a) arm VH and VL domains corresponding to SEQ ID NO:355 and SEQ ID NO:359, respectively, and

anti-FX(a) arm VH and VL domains corresponding to SEQ ID NO:467 and SEQ ID NO:471, respectively.

In one embodiment the bispecific antibody comprises anti-FIX(a) arm VH and VL domains corresponding to SEQ ID NO:347 and SEQ ID NO:351, respectively, and

anti-FX(a) arm VH and VL domains corresponding to SEQ ID NO:467 and SEQ ID NO:471, respectively.

In one embodiment the bispecific antibody comprises anti-FIX(a) arm VH and VL domains corresponding to SEQ ID NO:363 and SEQ ID NO:367, respectively, and

anti-FX(a) arm VH and VL domains corresponding to SEQ ID NO:467 and SEQ ID NO:471, respectively.

In one embodiment the bispecific antibody comprises anti-FIX(a) arm VH and VL domains corresponding to SEQ ID NO:371 and SEQ ID NO:375, respectively, and

anti-FX(a) arm VH and VL domains corresponding to SEQ ID NO:467 and SEQ ID NO:471, respectively.

In one embodiment the bispecific antibody comprises anti-FIX(a) arm VH and VL domains corresponding to SEQ ID NO:379 and SEQ ID NO:383, respectively, and

anti-FX(a) arm VH and VL domains corresponding to SEQ ID NO:467 and SEQ ID NO:471, respectively.

In one embodiment the bispecific antibody comprises anti-FIX(a) arm VH and VL domains corresponding to SEQ ID NO:387 and SEQ ID NO:391, respectively, and

anti-FX(a) arm VH and VL domains corresponding to SEQ ID NO:467 and SEQ ID NO:471, respectively.

In one embodiment the bispecific antibody comprises anti-FIX(a) arm VH and VL domains corresponding to SEQ ID NO:403 and SEQ ID NO:407, respectively, and

anti-FX(a) arm VH and VL domains corresponding to SEQ ID NO:467 and SEQ ID NO:471, respectively.

In one embodiment the bispecific antibody comprises anti-FIX(a) arm VH and VL domains corresponding to SEQ ID NO:395 and SEQ ID NO:399, respectively, and

anti-FX(a) arm VH and VL domains corresponding to SEQ ID NO:467 and SEQ ID NO:471, respectively.

In one embodiment the bispecific antibody comprises anti-FIX(a) arm VH and VL domains corresponding to SEQ ID NO:419 and SEQ ID NO:423, respectively, and

anti-FX(a) arm VH and VL domains corresponding to SEQ ID NO:467 and SEQ ID NO:471, respectively.

In one embodiment the bispecific antibody comprises anti-FIX(a) arm VH and VL domains corresponding to SEQ ID NO:411 and SEQ ID NO:415, respectively, and

anti-FX(a) arm VH and VL domains corresponding to SEQ ID NO:467 and SEQ ID NO:471, respectively.

In one embodiment the bispecific antibody comprises anti-FIX(a) arm VH and VL domains corresponding to SEQ ID NO:427 and SEQ ID NO:431, respectively, and

anti-FX(a) arm VH and VL domains corresponding to SEQ ID NO:467 and SEQ ID NO:471, respectively.

In one embodiment the bispecific antibody comprises anti-FIX(a) arm VH and VL domains corresponding to SEQ ID NO:131 and SEQ ID NO:135, respectively, and

anti-FX(a) arm VH and VL domains corresponding to SEQ ID NO:467 and SEQ ID NO:471, respectively.

In one embodiment the bispecific antibody comprises anti-FIX(a) arm VH and VL domains corresponding to SEQ ID NO:435 and SEQ ID NO:439, respectively, and

anti-FX(a) arm VH and VL domains corresponding to SEQ ID NO:467 and SEQ ID NO:471, respectively.

In one embodiment the bispecific antibody comprises anti-FIX(a) arm VH and VL domains corresponding to SEQ ID NO:443 and SEQ ID NO:447, respectively, and

anti-FX(a) arm VH and VL domains corresponding to SEQ ID NO:467 and SEQ ID NO:471, respectively.

In one embodiment the bispecific antibody comprises anti-FIX(a) arm VH and VL domains corresponding to SEQ ID NO:451 and SEQ ID NO:455, respectively, and

anti-FX(a) arm VH and VL domains corresponding to SEQ ID NO:467 and SEQ ID NO:471, respectively.

In one embodiment the bispecific antibody comprises anti-FIX(a) arm VH and VL domains corresponding to SEQ ID NO:51 and SEQ ID NO:55, respectively, and

anti-FX(a) arm VH and VL domains corresponding to SEQ ID NO:771 and SEQ ID NO:775, respectively.

In one embodiment the bispecific antibody comprises anti-FIX(a) arm VH and VL domains corresponding to SEQ ID NO:51 and SEQ ID NO:55, respectively, and

anti-FX(a) arm VH and VL domains corresponding to SEQ ID NO:707 and SEQ ID NO:711, respectively.

In one embodiment the bispecific antibody comprises anti-FIX(a) arm VH and VL domains corresponding to SEQ ID NO:51 and SEQ ID NO:55, respectively, and

anti-FX(a) arm VH and VL domains corresponding to SEQ ID NO:779 and SEQ ID NO:783, respectively.

In one embodiment the bispecific antibody comprises anti-FIX(a) arm VH and VL domains corresponding to SEQ ID NO:51 and SEQ ID NO:55, respectively, and

anti-FX(a) arm VH and VL domains corresponding to SEQ ID NO:763 and SEQ ID NO:767, respectively.

In one embodiment the bispecific antibody comprises anti-FIX(a) arm VH and VL domains corresponding to SEQ ID NO:51 and SEQ ID NO:55, respectively, and

anti-FX(a) arm VH and VL domains corresponding to SEQ ID NO:787 and SEQ ID NO:791, respectively.

In one embodiment the bispecific antibody comprises anti-FIX(a) arm VH and VL domains corresponding to SEQ ID NO:51 and SEQ ID NO:55, respectively, and

anti-FX(a) arm VH and VL domains corresponding to SEQ ID NO:795 and SEQ ID NO:799, respectively.

In one embodiment the bispecific antibody comprises anti-FIX(a) arm VH and VL domains corresponding to SEQ ID NO:51 and SEQ ID NO:55, respectively, and

anti-FX(a) arm VH and VL domains corresponding to SEQ ID NO:819 and SEQ ID NO:823, respectively.

In one embodiment the bispecific antibody comprises anti-FIX(a) arm VH and VL domains corresponding to SEQ ID NO:51 and SEQ ID NO:55, respectively, and

anti-FX(a) arm VH and VL domains corresponding to SEQ ID NO:803 and SEQ ID NO:807, respectively.

In one embodiment the bispecific antibody comprises anti-FIX(a) arm VH and VL domains corresponding to SEQ ID NO:51 and SEQ ID NO:55, respectively, and

anti-FX(a) arm VH and VL domains corresponding to SEQ ID NO:827 and SEQ ID NO:831, respectively.

In one embodiment the bispecific antibody comprises anti-FIX(a) arm VH and VL domains corresponding to SEQ ID NO:51 and SEQ ID NO:55, respectively, and

anti-FX(a) arm VH and VL domains corresponding to SEQ ID NO:811 and SEQ ID NO:815, respectively.

In one embodiment the bispecific antibody comprises anti-FIX(a) arm VH and VL domains corresponding to SEQ ID NO:51 and SEQ ID NO:55, respectively, and

anti-FX(a) arm VH and VL domains corresponding to SEQ ID NO:835 and SEQ ID NO:839, respectively.

In one embodiment the bispecific antibody comprises anti-FIX(a) arm VH and VL domains corresponding to SEQ ID NO:51 and SEQ ID NO:55, respectively, and

anti-FX(a) arm VH and VL domains corresponding to SEQ ID NO:715 and SEQ ID NO:719, respectively.

In one embodiment the bispecific antibody comprises anti-FIX(a) arm VH and VL domains corresponding to SEQ ID NO:51 and SEQ ID NO:55, respectively, and

anti-FX(a) arm VH and VL domains corresponding to SEQ ID NO:843 and SEQ ID NO:847, respectively.

In one embodiment the bispecific antibody comprises anti-FIX(a) arm VH and VL domains corresponding to SEQ ID NO:51 and SEQ ID NO:55, respectively, and

anti-FX(a) arm VH and VL domains corresponding to SEQ ID NO:851 and SEQ ID NO:855, respectively.

In one embodiment the bispecific antibody comprises anti-FIX(a) arm VH and VL domains corresponding to SEQ ID NO:51 and SEQ ID NO:55, respectively, and

anti-FX(a) arm VH and VL domains corresponding to SEQ ID NO:875 and SEQ ID NO:879, respectively.

In one embodiment the bispecific antibody comprises anti-FIX(a) arm VH and VL domains corresponding to SEQ ID NO:51 and SEQ ID NO:55, respectively, and

anti-FX(a) arm VH and VL domains corresponding to SEQ ID NO:859 and SEQ ID NO:863, respectively. In one embodiment the bispecific antibody comprises anti-FIX(a) arm VH and VL domains corresponding to SEQ ID NO:51 and SEQ ID NO:55, respectively, and

anti-FX(a) arm VH and VL domains corresponding to SEQ ID NO:883 and SEQ ID NO:887, respectively. In one embodiment the bispecific antibody comprises anti-FIX(a) arm VH and VL domains corresponding to SEQ ID NO:51 and SEQ ID NO:55, respectively, and

anti-FX(a) arm VH and VL domains corresponding to SEQ ID NO:867 and SEQ ID NO:871, respectively. In one embodiment the bispecific antibody comprises anti-FIX(a) arm VH and VL domains corresponding to SEQ ID NO:51 and SEQ ID NO:55, respectively, and

anti-FX(a) arm VH and VL domains corresponding to SEQ ID NO:891 and SEQ ID NO:895, respectively. In one embodiment the bispecific antibody comprises anti-FIX(a) arm VH and VL domains corresponding to SEQ ID NO:51 and SEQ ID NO:55, respectively, and

anti-FX(a) arm VH and VL domains corresponding to SEQ ID NO:723 and SEQ ID NO:727, respectively. In one embodiment the bispecific antibody comprises anti-FIX(a) arm VH and VL domains corresponding to SEQ ID NO:51 and SEQ ID NO:55, respectively, and

anti-FX(a) arm VH and VL domains corresponding to SEQ ID NO:899 and SEQ ID NO:903, respectively. In one embodiment the bispecific antibody comprises anti-FIX(a) arm VH and VL domains corresponding to SEQ ID NO:51 and SEQ ID NO:55, respectively, and

anti-FX(a) arm VH and VL domains corresponding to SEQ ID NO:923 and SEQ ID NO:927, respectively.

In one embodiment the bispecific antibody comprises anti-FIX(a) arm VH and VL domains corresponding to SEQ ID NO:51 and SEQ ID NO:55, respectively, and

anti-FX(a) arm VH and VL domains corresponding to SEQ ID NO:907 and SEQ ID NO:911, respectively.

In one embodiment the bispecific antibody comprises anti-FIX(a) arm VH and VL domains corresponding to SEQ ID NO:51 and SEQ ID NO:55, respectively, and

anti-FX(a) arm VH and VL domains corresponding to SEQ ID NO:731 and SEQ ID NO:735, respectively.

In one embodiment the bispecific antibody comprises anti-FIX(a) arm VH and VL domains corresponding to SEQ ID NO:51 and SEQ ID NO:55, respectively, and

anti-FX(a) arm VH and VL domains corresponding to SEQ ID NO:915 and SEQ ID NO:919, respectively.

In one embodiment the bispecific antibody comprises anti-FIX(a) arm VH and VL domains corresponding to SEQ ID NO:51 and SEQ ID NO:55, respectively, and

anti-FX(a) arm VH and VL domains corresponding to SEQ ID NO:931 and SEQ ID NO:935, respectively.

In one embodiment the bispecific antibody comprises anti-FIX(a) arm VH and VL domains corresponding to SEQ ID NO:51 and SEQ ID NO:55, respectively, and

anti-FX(a) arm VH and VL domains corresponding to SEQ ID NO:939 and SEQ ID NO:943, respectively.

In one embodiment the bispecific antibody comprises anti-FIX(a) arm VH and VL domains corresponding to SEQ ID NO:51 and SEQ ID NO:55, respectively, and

anti-FX(a) arm VH and VL domains corresponding to SEQ ID NO:963 and SEQ ID NO:967, respectively.

In one embodiment the bispecific antibody comprises anti-FIX(a) arm VH and VL domains corresponding to SEQ ID NO:51 and SEQ ID NO:55, respectively, and

anti-FX(a) arm VH and VL domains corresponding to SEQ ID NO:971 and SEQ ID NO:975, respectively.

In one embodiment the bispecific antibody comprises anti-FIX(a) arm VH and VL domains corresponding to SEQ ID NO:51 and SEQ ID NO:55, respectively, and

anti-FX(a) arm VH and VL domains corresponding to SEQ ID NO:947 and SEQ ID NO:951, respectively.

In one embodiment the bispecific antibody comprises anti-FIX(a) arm VH and VL domains corresponding to SEQ ID NO:51 and SEQ ID NO:55, respectively, and

anti-FX(a) arm VH and VL domains corresponding to SEQ ID NO:739 and SEQ ID NO:743, respectively.

In one embodiment the bispecific antibody comprises anti-FIX(a) arm VH and VL domains corresponding to SEQ ID NO:51 and SEQ ID NO:55, respectively, and

anti-FX(a) arm VH and VL domains corresponding to SEQ ID NO:955 and SEQ ID NO:959, respectively.

In one embodiment the bispecific antibody comprises anti-FIX(a) arm VH and VL domains corresponding to SEQ ID NO:51 and SEQ ID NO:55, respectively, and

anti-FX(a) arm VH and VL domains corresponding to SEQ ID NO:979 and SEQ ID NO:983, respectively.

In one embodiment the bispecific antibody comprises anti-FIX(a) arm VH and VL domains corresponding to SEQ ID NO:51 and SEQ ID NO:55, respectively, and

anti-FX(a) arm VH and VL domains corresponding to SEQ ID NO:987 and SEQ ID NO:991, respectively.

In one embodiment the bispecific antibody comprises anti-FIX(a) arm VH and VL domains corresponding to SEQ ID NO:51 and SEQ ID NO:55, respectively, and

anti-FX(a) arm VH and VL domains corresponding to SEQ ID NO:1011 and SEQ ID NO:1015, respectively.

In one embodiment the bispecific antibody comprises anti-FIX(a) arm VH and VL domains corresponding to SEQ ID NO:51 and SEQ ID NO:55, respectively, and

anti-FX(a) arm VH and VL domains corresponding to SEQ ID NO:1019 and SEQ ID NO:1023, respectively.

In one embodiment the bispecific antibody comprises anti-FIX(a) arm VH and VL domains corresponding to SEQ ID NO:51 and SEQ ID NO:55, respectively, and

anti-FX(a) arm VH and VL domains corresponding to SEQ ID NO:995 and SEQ ID NO:999, respectively.

In one embodiment the bispecific antibody comprises anti-FIX(a) arm VH and VL domains corresponding to SEQ ID NO:51 and SEQ ID NO:55, respectively, and

anti-FX(a) arm VH and VL domains corresponding to SEQ ID NO:747 and SEQ ID NO:751, respectively.

In one embodiment the bispecific antibody comprises anti-FIX(a) arm VH and VL domains corresponding to SEQ ID NO:51 and SEQ ID NO:55, respectively, and

anti-FX(a) arm VH and VL domains corresponding to SEQ ID NO:1003 and SEQ ID NO:1007, respectively.

In one embodiment the bispecific antibody comprises anti-FIX(a) arm VH and VL domains corresponding to SEQ ID NO:51 and SEQ ID NO:55, respectively, and

anti-FX(a) arm VH and VL domains corresponding to SEQ ID NO:1027 and SEQ ID NO:1031, respectively.

In one embodiment the bispecific antibody comprises anti-FIX(a) arm VH and VL domains corresponding to SEQ ID NO:51 and SEQ ID NO:55, respectively, and

anti-FX(a) arm VH and VL domains corresponding to SEQ ID NO:1051 and SEQ ID NO:1055, respectively.

In one embodiment the bispecific antibody comprises anti-FIX(a) arm VH and VL domains corresponding to SEQ ID NO:51 and SEQ ID NO:55, respectively, and

anti-FX(a) arm VH and VL domains corresponding to SEQ ID NO:1035 and SEQ ID NO:1039, respectively.

In one embodiment the bispecific antibody comprises anti-FIX(a) arm VH and VL domains corresponding to SEQ ID NO:51 and SEQ ID NO:55, respectively, and

anti-FX(a) arm VH and VL domains corresponding to SEQ ID NO:1059 and SEQ ID NO:1063, respectively.

In one embodiment the bispecific antibody comprises anti-FIX(a) arm VH and VL domains corresponding to SEQ ID NO:51 and SEQ ID NO:55, respectively, and

anti-FX(a) arm VH and VL domains corresponding to SEQ ID NO:1043 and SEQ ID NO:1047, respectively.

In one embodiment the bispecific antibody comprises anti-FIX(a) arm VH and VL domains corresponding to SEQ ID NO:51 and SEQ ID NO:55, respectively, and

anti-FX(a) arm VH and VL domains corresponding to SEQ ID NO:1067 and SEQ ID NO:1071, respectively.

In one embodiment the bispecific antibody comprises anti-FIX(a) arm VH and VL domains corresponding to SEQ ID NO:51 and SEQ ID NO:55, respectively, and

anti-FX(a) arm VH and VL domains corresponding to SEQ ID NO:1075 and SEQ ID NO:1079, respectively.

In one embodiment the bispecific antibody comprises anti-FIX(a) arm VH and VL domains corresponding to SEQ ID NO:51 and SEQ ID NO:55, respectively, and

anti-FX(a) arm VH and VL domains corresponding to SEQ ID NO:1099 and SEQ ID NO:1103, respectively.

In one embodiment the bispecific antibody comprises anti-FIX(a) arm VH and VL domains corresponding to SEQ ID NO:51 and SEQ ID NO:55, respectively, and

anti-FX(a) arm VH and VL domains corresponding to SEQ ID NO:755 and SEQ ID NO:759, respectively.

In one embodiment the bispecific antibody comprises anti-FIX(a) arm VH and VL domains corresponding to SEQ ID NO:51 and SEQ ID NO:55, respectively, and

anti-FX(a) arm VH and VL domains corresponding to SEQ ID NO:1107 and SEQ ID NO:1111, respectively.

In one embodiment the bispecific antibody comprises anti-FIX(a) arm VH and VL domains corresponding to SEQ ID NO:51 and SEQ ID NO:55, respectively, and

anti-FX(a) arm VH and VL domains corresponding to SEQ ID NO:1083 and SEQ ID NO:1087, respectively.

In one embodiment the bispecific antibody comprises anti-FIX(a) arm VH and VL domains corresponding to SEQ ID NO:51 and SEQ ID NO:55, respectively, and

anti-FX(a) arm VH and VL domains corresponding to SEQ ID NO:1115 and SEQ ID NO:1119, respectively.

In one embodiment the bispecific antibody comprises anti-FIX(a) arm VH and VL domains corresponding to SEQ ID NO:51 and SEQ ID NO:55, respectively, and

anti-FX(a) arm VH and VL domains corresponding to SEQ ID NO:1091 and SEQ ID NO:1095, respectively.

In one embodiment the bispecific antibody comprises anti-FIX(a) arm VH and VL domains corresponding to SEQ ID NO:51 and SEQ ID NO:55, respectively, and

anti-FX(a) arm VH and VL domains corresponding to SEQ ID NO:1123 and SEQ ID NO:1127, respectively.

In one embodiment the bispecific antibody comprises anti-FIX(a) arm VH and VL domains corresponding to SEQ ID NO:51 and SEQ ID NO:55, respectively, and

anti-FX(a) arm VH and VL domains corresponding to SEQ ID NO:1131 and SEQ ID NO:1135, respectively.

In one embodiment the bispecific antibody comprises anti-FIX(a) arm VH and VL domains corresponding to SEQ ID NO:51 and SEQ ID NO:55, respectively, and

anti-FX(a) arm VH and VL domains corresponding to SEQ ID NO:1155 and SEQ ID NO:1159, respectively.

In one embodiment the bispecific antibody comprises anti-FIX(a) arm VH and VL domains corresponding to SEQ ID NO:51 and SEQ ID NO:55, respectively, and

anti-FX(a) arm VH and VL domains corresponding to SEQ ID NO:1163 and SEQ ID NO:1167, respectively.

In one embodiment the bispecific antibody comprises anti-FIX(a) arm VH and VL domains corresponding to SEQ ID NO:51 and SEQ ID NO:55, respectively, and

anti-FX(a) arm VH and VL domains corresponding to SEQ ID NO:1139 and SEQ ID NO:1143, respectively.

In one embodiment the bispecific antibody comprises anti-FIX(a) arm VH and VL domains corresponding to SEQ ID NO:51 and SEQ ID NO:55, respectively, and

anti-FX(a) arm VH and VL domains corresponding to SEQ ID NO:1171 and SEQ ID NO:1175, respectively.

In one embodiment the bispecific antibody comprises anti-FIX(a) arm VH and VL domains corresponding to SEQ ID NO:51 and SEQ ID NO:55, respectively, and

anti-FX(a) arm VH and VL domains corresponding to SEQ ID NO:1147 and SEQ ID NO:1151, respectively.

In one embodiment the bispecific antibody comprises anti-FIX(a) arm VH and VL domains corresponding to SEQ ID NO:51 and SEQ ID NO:55, respectively, and

anti-FX(a) arm VH and VL domains corresponding to SEQ ID NO:1179 and SEQ ID NO:1183, respectively.

In one embodiment the bispecific antibody comprises anti-FIX(a) arm VH and VL domains corresponding to SEQ ID NO:51 and SEQ ID NO:55, respectively, and

anti-FX(a) arm VH and VL domains corresponding to SEQ ID NO:1187 and SEQ ID NO:1191, respectively.

In one embodiment the bispecific antibody comprises anti-FIX(a) arm VH and VL domains corresponding to SEQ ID NO:51 and SEQ ID NO:55, respectively, and

anti-FX(a) arm VH and VL domains corresponding to SEQ ID NO:547 and SEQ ID NO:551, respectively.

In one embodiment the bispecific antibody comprises anti-FIX(a) arm VH and VL domains corresponding to SEQ ID NO:1202 and SEQ ID NO:1206, respectively, and

anti-FX(a) arm VH and VL domains corresponding to SEQ ID NO:467 and SEQ ID NO:471, respectively.

In one embodiment the bispecific antibody comprises anti-FIX(a) arm VH and VL domains corresponding to SEQ ID NO:1210 and SEQ ID NO:1214, respectively, and

anti-FX(a) arm VH and VL domains corresponding to SEQ ID NO:467 and SEQ ID NO:471, respectively.

In one embodiment the bispecific antibody comprises anti-FIX(a) arm VH and VL domains corresponding to SEQ ID NO:1218 and SEQ ID NO:1222, respectively, and

anti-FX(a) arm VH and VL domains corresponding to SEQ ID NO:467 and SEQ ID NO:471, respectively.

In one embodiment the bispecific antibody comprises anti-FIX(a) arm VH and VL domains corresponding to SEQ ID NO:1226 and SEQ ID NO:1230, respectively, and

anti-FX(a) arm VH and VL domains corresponding to SEQ ID NO:467 and SEQ ID NO:471, respectively.

In one embodiment the bispecific antibody comprises anti-FIX(a) arm VH and VL domains corresponding to SEQ ID NO:1234 and SEQ ID NO:1238, respectively, and

anti-FX(a) arm VH and VL domains corresponding to SEQ ID NO:467 and SEQ ID NO:471, respectively.

In one embodiment the bispecific antibody comprises anti-FIX(a) arm VH and VL domains corresponding to SEQ ID NO:1242 and SEQ ID NO:1246, respectively, and

anti-FX(a) arm VH and VL domains corresponding to SEQ ID NO:467 and SEQ ID NO:471, respectively.

In one embodiment the paratope of an anti-FIX(a) antibody or antigen-binding fragment thereof of the invention comprises amino acid residues H30, D31, W53, D56, S102, S104, Y106 and N107 in the heavy chain variable domain (SEQ ID NO:67) and residues Y91 and S92 in the light chain variable domain (SEQ ID NO:71).

In one embodiment an anti-FIX(a) antibody or antigen-binding fragment thereof of the invention comprises one, two or three amino acid substitutions or deletions within the group of paratope amino acid residues H30, D31, W53, D56, S102, S104, Y106 and N107 in the heavy chain variable domain (SEQ ID NO:67) and residues Y91 and S92 in the light chain variable domain (SEQ ID NO:71).

In another embodiment the paratope of an anti-FIX(a) antibody or antigen-binding fragment thereof of the invention comprises amino acid residues D30, D31, W53, S102, S104 and N107 in the heavy chain variable domain (SEQ ID NO:35) and residues Y91 and S92 in the light chain variable domain (SEQ ID NO:39).

In another embodiment an anti-FIX(a) antibody of the invention comprises one, two or three amino acid substitutions or deletions within the group of paratope amino acid residues: D30, D31, W53, S102, S104 and N107 in the heavy chain variable domain (SEQ ID NO:35) and residues Y91 and S92 in the light chain variable domain (SEQ ID NO:39).

In one embodiment the CDR sequences of an antibody or antigen-binding fragment thereof of the invention may be described by anti-FIX(a) paratope amino acid residues being part of the CDRs.

In one such embodiment the anti-FIX(a) paratope CDRs are

VH CDR1 (based on SEQ ID NO: 68):
DXXXX
VH CDR2 (based on SEQ ID NO: 69):
XXXWXXDXXXXXXXXXX
VH CDR3 (based on SEQ ID NO: 70):
XXXSXSXYNXXXX
VL CDR1 (based on SEQ ID NO: 72):
XXXXXXXXXXX
VL CDR2 (based on SEQ ID NO: 73):
XXXXXXX
VL CDR3 (based on SEQ ID NO: 74):
XXYSXXXXX

wherein paratope amino acid residues are in bold, and X represents a naturally occurring amino acid residue.

In another such embodiment the anti-FIX(a) paratope CDRs are

VH CDR1 (based on SEQ ID NO: 36):
DXXXX
VH CDR2 (based on SEQ ID NO: 37):
XXXWXXXXXXXXXXXXX
VH CDR3 (based on SEQ ID NO: 38):
XXXSXSXXNXXXX
VL CDR1 (based on SEQ ID NO: 40):
XXXXXXXXXXX
VL CDR2 (based on SEQ ID NO: 41):
XXXXXXX
VL CDR3 (based on SEQ ID NO: 42):
XXYSXXXXX

wherein paratope amino acid residues are in bold, and X represents a naturally occurring amino acid residue.

In another such embodiment the anti-FIX(a) paratope CDRs are

VH CDR1 (based on SEQ ID NO: 52):
DXXXX
VH CDR2 (based on SEQ ID NO: 53):
XXXWXXXXXXXXXXXXX
VH CDR3 (based on SEQ ID NO: 54):
XXXSXSXYNXXXX
VL CDR1 (based on SEQ ID NO: 56):
XXXXXXXXXXX
VL CDR2 (based on SEQ ID NO: 57):
XXXXXXX
VL CDR3 (based on SEQ ID NO: 58):
XXYSXXXXX

wherein paratope amino acid residues are in bold, and X represents a naturally occurring amino acid residue.

In one embodiment the paratope of an anti-FX(a) antibody of the invention comprises residues K23, S25, G26, Y27, F29, W33, D52, S54, D55, F57, S77, H100, Y101, Y102, N103 and S104 in the heavy chain variable domain (SEQ ID NO:467) and residues V29, S30, S31, Y33, Y50, Q52, S54, R55, R57 and D94 in the light chain variable domain (SEQ ID NO:471).

In another embodiment an anti-FX(a) antibody of the invention comprises one, two or three amino acid substitutions or deletions within the group of paratope residues K23, S25, G26, Y27, F29, W33, D52, S54, D55, F57, S77, H100, Y101, Y102, N103 and S104 in the heavy chain variable domain (SEQ ID NO:467) and residues V29, S30, S31, Y33, Y50, Q52, S54, R55, R57 and D94 in the light chain variable domain (SEQ ID NO:471).

In another embodiment the paratope of an anti-FX(a) antibody of the invention comprises residues K23, G24, S25, G26, Y27, W33, D52, S54, D55, Y57, S77, L99, H100, Y101, Y102, N103 and S104 in the variable heavy chain domain (SEQ ID NO:483) and residues S30, S31, Y33, Y50, Q52, S54, R55, R57, Y92 and D94 in the light chain variable domain (SEQ ID NO:487).

In another embodiment an anti-FX(a) antibody of the invention comprises one, two or three amino acid substitutions or deletions within the group of paratope residues K23, G24, S25, G26, Y27, W33, D52, S54, D55, Y57, S77, L99, H100, Y101, Y102, N103 and S104 in the variable heavy chain domain (SEQ ID NO:483) and residues S30, S31, Y33, Y50, Q52, S54, R55, R57, Y92 and D94 in the light chain variable domain (SEQ ID NO:487).

In one embodiment the CDR sequences of such an antibody may be described by anti-FX(a) paratope amino acid residues being part of the CDRs.

In one such embodiment the anti-FX(a) paratope CDRs are

VH CDR1 (based on SEQ ID NO: 468):
XXBXX
VH CDR2 (based on SEQ ID NO: 469):
XXBXBXFBXXXXXXXX
VH CDR3 (based on SEQ ID NO: 470):
XHYYNSXXXXX
VL CDR1 (based on SEQ ID NO: 472):
XXXXXVSSXYXX
VL CDR2 (based on SEQ ID NO: 473):
XQXSRXR
VL CDR3 (based on SEQ ID NO: 474):
XXXXDXXXXX

wherein paratope amino acid residues are in bold, and X represents a naturally occurring amino acid residue.

In another such embodiment the anti-FX(a) paratope CDRs are

VH CDR1 (based on SEQ ID NO: 484):
XXWXX
VH CDR2 (based on SEQ ID NO: 485):
XXDXSDXYXXXXXXXXX
VH CDR3 (based on SEQ ID NO: 486):
LHYYNSXXXXX
VL CDR1 (based on SEQ ID NO: 488):
XXXXXXXSSXYXX
VL CDR2 (based on SEQ ID NO: 489):
XQXSRXR
VL CDR3 (based on SEQ ID NO: 490):
XXYXDXXXXX

wherein paratope amino acid residues are in bold, and X represents a naturally occurring amino acid residue.

In one embodiment an antibody of the invention is a multispecific antibody or antigen-binding fragment thereof capable of stimulating the enzymatic activity of FIXa towards FX comprising a first antigen-binding site capable of binding to FIX (SEQ ID NO:1) and/or the activated form thereof (FIXa), and a second antigen-binding site capable of binding to FX (SEQ ID NO:2) and/or the activated form thereof (FXa).

In one such embodiment the first antigen-binding site comprises a paratope comprising amino acid residues D30, D31, W53, S102, S104 and N107 in the heavy chain variable domain (SEQ ID NO:35) and amino acid residues Y91 and S92 in the light chain variable domain (SEQ ID NO:39), and the second antigen-binding site comprises a paratope comprising amino acid residues K23, G24, S25, G26, Y27, W33, D52, S54, D55, Y57, S77, L99, H100, Y101, Y102, N103 and S104 in the variable heavy chain domain (SEQ ID NO:483) and amino acid residues S30, S31, Y33, Y50, Q52, S54, R55, R57, Y92 and D94 in the light chain variable domain (SEQ ID NO:487).

In one such embodiment the first antigen-binding site comprises a paratope comprising amino acid residues H30, D31, W53, D56, S102, S104, Y106 and N107 in the heavy chain variable domain (SEQ ID NO:67) and amino acid residues Y91 and S92 in the light chain variable domain (SEQ ID NO:71), and the second antigen-binding site comprises a paratope comprising amino acid residues K23, G24, S25, G26, Y27, W33, D52, S54, D55, Y57, S77, L99, H100, Y101, Y102, N103 and S104 in the variable heavy chain domain (SEQ ID NO:483) and amino acid residues S30, S31, Y33, Y50, Q52, S54, R55, R57, Y92 and D94 in the light chain variable domain (SEQ ID NO:487).

In one such embodiment the first antigen-binding site comprises a paratope comprising amino acid residues H30, D31, W53, D56, S102, S104, Y106 and N107 in the heavy chain variable domain (SEQ ID NO:67) and amino acid residues Y91 and S92 in the light chain variable domain (SEQ ID NO:71), and the second antigen-binding site comprises a paratope comprising amino acid residues K23, S25, G26, Y27, F29, W33, D52, S54, D55, F57, S77, H100, Y101, Y102, N103 and S104 in the heavy chain variable domain (SEQ ID NO:467) and amino acid residues V29, S30, S31, Y33, Y50, Q52, S54, R55, R57 and D94 in the light chain variable domain (SEQ ID NO:471).

In one such embodiment the first antigen-binding site comprises a paratope comprising amino acid residues D30, D31, W53, S102, S104 and N107 in the heavy chain variable domain (SEQ ID NO:35) and amino acid residues Y91 and S92 in the light chain variable domain (SEQ ID NO:39), and the second antigen-binding site comprises a paratope comprising amino acid residues K23, S25, G26, Y27, F29, W33, D52, S54, D55, F57, S77, H100, Y101, Y102, N103 and S104 in the heavy chain variable domain (SEQ ID NO:467) and amino acid residues V29, S30, S31, Y33, Y50, Q52, S54, R55, R57 and D94 in the light chain variable domain (SEQ ID NO:471).

In one such embodiment the first antigen-binding site comprises a paratope comprising amino acid residues H30, D31, W53, S102, S104, Y106 and N107 in the heavy chain variable domain (SEQ ID NO:51) and amino acid residues Y91 and S92 in the light chain variable domain (SEQ ID NO:55), and the second antigen-binding site comprises a paratope comprising amino acid residues K23, S25, G26, Y27, F29, W33, D52, S54, D55, F57, S77, H100, Y101, Y102, N103 and S104 in the heavy chain variable domain (SEQ ID NO:467) and amino acid residues V29, S30, S31, Y33, Y50, Q52, S54, R55, R57 and D94 in the light chain variable domain (SEQ ID NO:471).

In one embodiment the antibody is a bispecific antibody capable of binding to FIX(a) and FX(a).

In one embodiment an antibody of the invention is capable of binding FIXa with a higher affinity than that with which it binds FIX.

In one embodiment an antibody of the invention is capable of increasing the enzymatic activity of FIXa towards FX.

In one such embodiment an antibody of the invention is capable of increasing the enzymatic activity of FIXa towards FX as measured in a FXa generation assay using monovalent one-armed antibodies as described herein.

In one embodiment an antibody of the invention is capable of increasing the enzymatic activity of FIXa towards FX as measured in a FXa generation assay using bivalent antibodies as described herein.

In one embodiment the multispecific antibodies, such as bispecific antibodies, of the invention do not interfere with the effect of FVIII, such as recombinant FVIII administered to a patient suffering from haemophilia A, when said antibodies are used in clinically relevant dosages in the treatment of haemophilia A.

In one embodiment an antibody of the invention is not the anti-FIX antibody CLB-FIX 13 as described in Rohlena et al. (2003) J. Biol. Chem. 278(11):9394-9401. In one embodiment an antibody of the invention is not the anti-FIX antibody HIX-1 (IgG1 murine) (Merck KGaA, SigmaAldrich). In one embodiment an antibody or antigen-binding fragment thereof of the invention is not the anti-FIX antibody AHIX-5041 (IgG1) (Haematologic Technologies, Inc.).

In one embodiment an antibody or antigen-binding fragment thereof of the invention has reduced immunogenicity as compared to procoagulant antibodies of the art.

In a preferred embodiment antibodies of the invention were—unless otherwise stated or contradicted by context—expressed in the IgG4/kappa format.

The heavy chain constant domain regions (C_{H}1-C_{H}2-C_{H}3) were for the anti-FIX(a) arm human IgG4 with a S228P (EU-numbering) substitution and with truncation of the C-terminal lysine:

(SEQ ID NO: 1195)
ASTKGPSVFPLAPCSRSTSESTAALGCLVKDYFPEPVTVSWNSGALTSG
VHTFPAVLQSSGLYSLSSVVTVPSSSLGTKTYTCNVDHKPSNTKVDKRV
ESKYGPPCP [symbol] CPAPEFLGGPSVFLFPPKPKDTLMISRTPEVTCVVVDVS
QEDPEVQFNWYVDGVEVHNAKTKPREEQFNSTYRVVSVLTVLHQDWLNG
KEYKCKVSNKGLPSSIEKTISKAKGQPREPQVYTLPPSQEEMTKNQVSL
TCLVKGFYPSDIAVEWESNGQPENNYKTTPPVLDSDGSFFLYSRLTVDK
SRWQEGNVFSCSVMHEALHNHYTQKSLSLSLG.

In one embodiment the heavy chain constant domain regions (C_{H}1-C_{H}2-C_{H}3) were for the anti-FX(a) arm human IgG4 with the S228P substitution and with two additional substitutions, F405L and R409K (EU numbering), in the C_{H}3 domain to facilitate hetero-dimerization of the heavy chains (described in example 4) and with truncation of the C-terminal lysine:

(SEQ ID NO: 1196)
ASTKGPSVFPLAPCSRSTSESTAALGCLVKDYFPEPVTVSWNSGALTSGV
HTFPAVLQSSGLYSLSSVVTVPSSSLGTKTYTCNVDHKPSNTKVDKRVES
KYGPPCP [symbol] CPAPEFLGGPSVFLFPPKPKDTLMISRTPEVTCVVVDVSQED
PEVQFNWYVDGVEVHNAKTKPREEQFNSTYRVVSVLTVLHQDWLNGKEYK
CKVSNKGLPSSIEKTISKAKGQPREPQVYTLPPSQEEMTKNQVSLTCLVK
GFYPSDIAVEWESNGQPENNYKTTPPVLDSDGSF [symbol] LYS [symbol] LTVDKSRWQE
GNVFSCSVMHEALHNHYTQKSLSLSLG.

and, the light chain constant region (CL) was human kappa:

(SEQ ID NO: 1197)
RTVAAPSVFIFPPSDEQLKSGTASVVOLLNNFYPREAKVQWKVDNALQS
GNSQESVTEQDSKDSTYSLSSTLTLSKADYEKHKVYACEVTHQGLSSPV
TKSFNRGEC.

In another embodiment antibodies can also be expressed in the IgG4 format with heavy chain constant domain regions (C_{H}1-C_{H}2-C_{H}3) for the anti-FIX(a) arm carrying S228P, F405L and R409K substitutions and with heavy chain constant domain regions for the anti-FX(a) arm carrying the S228P substitution, with or without C-terminal lysine deletion.

In one embodiment antibodies can also be expressed in the IgG1/kappa format. In that case the heavy chain constant domain regions of the anti-FIX(a) arm is human IgG1 F405L with truncation of the C-terminal lysine:

(SEQ ID NO: 1198)
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKV
EPKSCDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDW
LNGKEYKCKVSNKALPAPIEKTISKAKGQPREPQVYTLPPSRDELTKNQ
VSLTCLVKGFYPSDIAVEWESNGQPENNYKTTPPVLDSDGSF [symbol] LYSK
LTVDKSRWQQGNVFSCSVMHEALHNHYTQKSLSLSPG.

and the heavy chain constant domain regions of the anti-FX(a) arm is human IgG1 K409R with truncation of the C-terminal lysine:

(SEQ ID NO: 1199)
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKV
EPKSCDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDW
LNGKEYKCKVSNKALPAPIEKTISKAKGQPREPQVYTLPPSRDELTKNQ
VSLTCLVKGFYPSDIAVEWESNGQPENNYKTTPPVLDSDGSFFLYS [symbol] LT
VDKSRWQQGNVFSCSVMHEALHNHYTQKSLSLSPG.

Antibodies can also be expressed in the IgG1 format with heavy chain constant domain regions (C_{H}1-C_{H}2-C_{H}3) for the anti-FIX(a) arm carrying the K409R substitution and with heavy chain constant domain regions for the anti-FX(a) arm carrying the F405L substitution, with or without C-terminal lysine deletion.

The constant domain regions may further comprise additional substitutions or other modifications e.g. to modulate effector functions, half-life or other properties.

The present disclosure also provides kits that comprise antibodies or antigen-binding fragments thereof as disclosed herein suitable for treatment as described herein. In some embodiments, a kit comprises (i) an antibody, such as a bispecific antibody or antigen-binding fragment thereof, pharmaceutical composition, nucleic acid, vector, or cell (e.g., a host cell) as disclosed herein, or a combination thereof, and (ii) instructions for use. A skilled person will readily recognize that the antibodies, bispecific molecules (e.g., bispecific antibodies), pharmaceutical compositions, nucleic acids, vectors, or cells (e.g., a host cell) disclosed herein, or combinations thereof can be readily incorporated into one of the established kit formats which are well known in the art.

### EXAMPLES

### List of Abbreviations

ACN: Acetonitrile

bimAb: Bispecific monoclonal Antibody

CDR: Complementarity Determining Region

EGR-CK: EGR-chloromethylketone

LC-MS Liquid chromatography-mass spectrometry

FACS: Fluorescence-activated cell sorting

FIX: Coagulation Factor IX

FIXa: Coagulation Factor IXa

FX: Coagulation Factor X

FXa: Coagulation Factor Xa

HA: Haemophilia A

HA-PPP: HA-induced human platelet-poor plasma

HA-PRP: HA-induced human platelet-rich plasma

hFIXa: human Coagulation Factor IXa

ITC: Isothermal Titration calorimetry

MACS: Magnetic-activated cell sorting

OA: One-armed

PCR: Polymerase Chain Reaction

SPR: Surface Plasmon Resonance

References to ACE910 should be understood as referring to an antibody having an amino acid sequence which is identical to that of ACE910.

### Example 1: Development of Anti-FIX(a) and Anti-FX(a) Antibodies

FIX(a) and FX(a) binding antibodies as disclosed herein were identified using various antibody development methods. In order to generate a diverse set of antibodies, immunisations of mice and rabbits were performed and phage display and Adimab yeast antibody expression platforms were also utilized.

Adimab Yeast Antibody Platform

The Adimab platform is a yeast antibody expression system encompassing a fully human naïve IgG1/kappa library with a diversity of 10¹⁰. The antibody selection process was directed using MACS and FACS based methods which allowed monitoring of applied selection criteria in real time. Since selections were based on MACS and FACS, labelled antigens (e.g. biotin) were needed. Selection campaigns were performed using biotin-labelled active-site inhibited hFIXa (FIXa-EGR-biotin), or antibody mediated immobilization of hFIXa. Hits were evaluated for binding using Bio-layer interferometry (Octet fortebio systems).

Phage Display

The utilized antibody phage display platform is a proprietary fully human Fab display library. The library has a size of 10¹⁰ and was constructed by a combinational approach utilizing chemical synthesis of the light chain, as well as the heavy chain CDR1 and CDR2, complemented with PCR amplification of the heavy chain CDR3 from human peripheral blood mononuclear cells. To maximise epitope diversity, different panning strategies were explored, including panning using biotinylated FIXa-EGR, FX, active-site inhibited FXa, or antigen capture using anti-FIXa antibodies. Initial hits were identified by phage ELISA. After sequence analysis, unique hits were cloned and recombinantly expressed as IgG1 antibodies, and ranked using SPR (Biacore) or Bio-layer interferometry (Octet fortebio systems).

In Vivo Platforms

Mice and rabbits were used for the generation of antibodies using in vivo platforms. For the generation of anti-FIX/FIXa antibodies, mice or rabbits were immunized with human FIXa, FIXa-EGR or FIX using standard protocols. The spleen cells from mice were fused with myeloma cells using standard techniques and the resulting antibody containing hybridoma supernatants were screened for binding to FIXa using ELISA. FIXa binding rabbit B-cells were single cell sorted using FACS by gating on cells binding randomly biotinylated FIXa-EGR (detected by streptavidin conjugated fluorophore). Sorted rabbit B cells were cultured for seven days in 384w plates using feeder cells and conditioned medium from splenocytes, prior to screening against FIXa in ELISA. Rabbit B-cells and mouse hybridoma clones, expressing FIXa binding antibody hits, were either used for VH/VL sequencing followed by recombinant expression (for rabbit or hybridoma mAbs) or further propagated for mAb production (mouse hybridomas).

For the generation of anti-FX antibodies, mice and rabbits were immunised with FX using standard protocols. Rabbit B-cells were isolated by FACS based single-cell sorting and using randomly biotinylated FX (detected by streptavidin conjugated flourophore) while spleen cells from immunized mice were used for standard hybridoma development. Resulting antibody producing B-cell or mouse hybridoma clones were screened for FX binding using ELISA and Octet fortebio systems. Rabbit B-cell or mouse hybridoma clones expressing antibody hits were either used for V_{H}/VL sequencing followed by recombinant expression (for rabbit or hybridoma mAbs) or further propagated for mAb production (mouse hybridomas)

Sequencing of Hybridoma-Derived Antibodies

Anti-FIXa and anti-FX antibody producing hybridomas were sequenced and expressed in HEK293 cells using standard techniques. Expressed antibodies were evaluated for antigen binding using Octet fortebio systems.

Total RNA was extracted from antibody producing clones and the variable domain (V_{H} and V_{L}) encoding DNA sequences were amplified using RT-PCR. V_{H} and V_{L} sequences were determined and inserted into a pTT-based mammalian expression vector (Durocher et al (2002) Nucleic Acid Res. 30: E9) or into a pcDNA3.4 mammalian expression vector (Invitrogen) containing antibody constant region encoding DNA sequences. For pTT/pcDNA3.4 mAb expression vectors, the V_{H} and V_{L} DNA sequences were inserted in-frame with human IgG1 or IgG₄ S228P (C_{H}1 C_{H}2 C_{H}3, optionally with additional amino acid substitutions and deletions, e.g. substitutions in the C_{H}3 domain and deletion of the C-terminal lysine) or human C_{L} kappa constant region encoding DNA sequences, respectively. For the corresponding pTT/pcDNA3.4 Fab expression vectors the V_{H} DNA sequences were inserted in-frame with human IgG₄ C_{H}1 encoding DNA sequences.

### Example 2: Recombinant Expression of Antibodies and Antibody Fab Fragments

Antibodies and antibody Fab fragments were expressed using transient transfection of HEK293 suspension cells (293Expi, Invitrogen) essentially following manufacturer's instructions. 293Expi cells were typically subcultivated every 3-4 days in Expi293F expression medium (Invitrogen, catalogue number A1435104) supplemented with 1% P/S (GIBCO catalogue number 15140-122). Expi293F cells were transfected at a cell density of 2.5-3 mill/mL using Expifectamine. For each litre of Expi293F cells, the transfection was performed by diluting a total of 1 mg of plasmid DNA (V_{H}-C_{H}1 (for Fab) or V_{H}-C_{H}1-C_{H}2-C_{H}3 (for mAb) and LC plasmids in 1:1 ratio) into 50 mL Optimem (GIBCO, cat. no. 51985-026, dilution A) and by diluting 2.7 mL Expifectamine into 50 mL Optimem (dilution B). For Fab and mAb producing co-transfections, V_{H}-C_{H}1 and LC plasmids (Fab) and V_{H}-C_{H}1-C_{H}2-C_{H}3 and LC plasmids (mAb), respectively, were used in a 1:1 ratio. Dilution A and B were mixed and incubated at room temperature for 10-20 minutes. The transfection mix was hereafter added to the Expi293F cells and cells were incubated at 37° C. in a humidified incubator with orbital rotation (85-125 rpm). One day post-transfection, transfected cells were supplemented with 5 ml of ExpiFectamine 293 Transfection Enhancer 1 and 50 ml of ExpiFectamine 293 Transfection Enhancer 2. Cell culture supernatants were typically harvested 4-5 days post-transfection by centrifugation followed by filtration.

### Example 3: Fab and Antibody Purification and Characterization

All purification steps were carried out at 4° C. For lab scale, Milli-Q water was used for buffer preparation. The HPLC system used for SE-HPLC analysis was Aglient 1100. Aggregation and LC/MS were assessed for QC.

Capturing of Fab was performed with HiTrap Protein G HP affinity chromatography with binding buffer in 1×PBS (10 mM Na2HPO4, 1.8 mM KH2PO4, 137 mM NaCl, 2.7 mM KCl), pH 7.4. One step elution was performed with 0.1M Glycine, pH 2.8. The final product was desalted via 52 mL GE Hiprep 16 desalting column into formulation buffer (25 mM HEPES, 150 mM NaCl) with pH 7.4 and concentrated by centrifugal ultrafilter (30KD C.O.) for storage at −80° C.

To assess the quality of the purified Fab, SDS-PAGE and high-performance size-exclusion chromatography (SE-HPLC) analysis were performed. Batches that did not meet the quality standards (e.g., <95% monomeric by SE-HPLC) were further purified by size-exclusion chromatography. LC/MS was carried out to verify identity of Fab protein. Molecular weights (MWs) of all Fab's were shown to be consistent with theoretical MW of heavy chain and light chain, respectively.

Antibody Purification and Characterization

Purification of the antibodies was conducted by affinity chromatography using a Protein A MabSelect SuRe resins (GE Healthcare, cat. no. 17-5438-01). For small-scale antibody productions, protein A based purification was performed in 96 well plates while for larger productions, the ÄktaExplorer chromatography system (GE Healthcare, cat. no. 18-1112-41) was used. The buffer systems used for the affinity purification step were 1) an equilibration buffer composed of 20 mM NaPhosphate pH 7.2, 150 mM NaCl and 2) an elution buffer composed of 10 mM Formic acid pH 3.5 and 3) a pH-adjustment buffer composed of 0.4 M NaPhosphate pH 9.0. Cell supernatants were applied directly without any adjustments onto a pre-equilibrated MabSelect SuRe column. The column was washed with approximately 10 column volumes of equilibration buffer and the antibodies were eluted isocratically in approx. 2-5 column volume of elution buffer. The pH of the pooled fractions was adjusted to neutral using the described pH-adjustment buffer immediately after elution.

The purified antibodies were characterized using different methods such as SDS-PAGE/Coomassie, size-exclusion high-pressure liquid-chromatography (SE-H PLC) and liquid-chromatography mass spectrometry (LC-MS) analyses. The SDS-PAGE/Coomassie analysis was performed using NuPage 4-12% Bis-Tris gels (Invitrogen, cat. no. NP0321BOX). Here, all antibodies displayed expected light chain and heavy chain components. Intact molecular mass determinations were performed using a Liquid Chromatography Electrospray Ionisation Time-of-Flight Mass Spectrometry method setup on an Agilent 6210 instrument and a desalting column MassPREP (Waters, cat. no. USRM10008656). The buffer system used was an equilibration buffer composed of 0.1% Formic acid in LC-MS graded-H₂O and an elution buffer composed of 0.1% formic acid in LC-MS graded-ACN. Analyses were performed with and without N-Glycosidase F (Roche Diagnostics, cat. no. 11365177001) and reducing agent (i.e. mercaptoethanol or DTT). All antibodies displayed expected intact molecular masses in accordance with sequence and one heavy chain N-glycan. Purity was determined based on SE-HPLC. The final protein purity was analysed based on SE-HPLC method setup on an Agilent LC 1100/1200 system and using a BIOSep-SEC-53000 300×7.8 mm column (Phenomenex, cat. no. 00H-2146-K0) and a running buffer composed of 200 mM NaPhosphate pH 6.9, 300 mM NaCl and 10% isopropanol. UV280 and fluorescence (Ex 280 nm/Em 354 nm) detectors was used for detection. The antibodies eluted as single symmetric peaks with retention times reflecting the size of the antibodies. Purity estimates were all between 95-99% for the different antibodies. To measure the final protein concentrations, a NanoDrop spectrophotometer (Thermo Scientific) was used together with specific extinction coefficients for each of the antibodies.

### Example 4: Bispecific Antibodies Prepared by In Vitro Assembly

Bispecific antibodies were generated by in vitro assembly of a first and a second antibody by the Duobody® method (Genmab) described (Labrijn et al. PNAS 2013, vol. 110, pp. 5145-5150) for bispecific human IgG1 antibodies and using a slightly modified variant for bispecific human IgG4 antibodies as detailed in the following.

For IgG1 the heavy chain constant region of the first antibody is human IgG1 K409R (anti-FIX/FIXa) and the heavy chain constant region of the second antibody is human IgG1 F405L (anti-FX/FXa). The IgG1 may be a IgG1 variant with reduced effector functions, as referred to earlier.

For human IgG4, the heavy chain constant region of the first antibody is IgG4 S228P (anti-FIX/FIXa) and the heavy chain constant region of the second antibody is IgG4 S228P F405L+R409K (anti-FX). The two parental antibodies are produced as described in Examples 1-3. The Fab arm exchange reaction is carried out in HEPES buffer (pH 7.4) under reducing conditions using 75 mM 2-mercaptoethylamine (2-MEA) and incubation at 30° C. for 4 hours.

### Example 5: Preparation of Monovalent (One-Armed) Antibodies

To avoid any potential avidity effects associated with conventional monospecific and bivalent antibodies, e.g. in FXa generation assays (Example 12) and in certain SPR-based experiments (Examples 10 and 11), a monovalent one-armed (OA) antibody format was used, as described by Martens et al.: A Novel One-Armed Anti-c-Met Antibody Inhibits Glioblastoma Growth In vivo. Clin. Cancer Res. 12, 6144-6152 (2006), where a full heavy chain, a truncated heavy chain (lacking the Fab region) and a light chain are co-expressed. Instead of co-expression of the three chains described by Martens et al. monovalent antibodies of the present invention were prepared using the Duobody® principle as described for bispecific antibodies (Example 4). Thus, monovalent antibodies were prepared by mixing a full monospecific and bivalent antibody and a truncated heavy chain dimer (formally derived from a full antibody by removing the Fab region) and allow exchange of chains to proceed under the same experimental conditions as described in Example 4. Formation of the monovalent antibody requires that the antibody and truncated heavy chain dimer carry appropriate complementary mutations to promote hetero-dimerization, i.e. F405L/K409R for human IgG1 and F405L+R409K/WT for human IgG4, as described in Example 4.

In case of monovalent antibodies of the IgG1 subtype the truncation of the heavy chain can be from the N-terminus to a position in-between Cys 220 and the upper hinge Cys 226 (EU numbering). A specific example of a truncated human IgG1 heavy chain is one where residues 1-220 are truncated.

In case of monovalent antibodies of the human IgG4 subtype the truncation of the heavy chain can be from the N-terminus to a position in-between Cys 200 and the upper hinge Cys 226 (EU numbering). A specific example of a truncated human IgG4 heavy chain is one where residues 1-214 are truncated.

### Example 6: Overview of Bispecific Antibody (Component) IDs and SEQ ID NOs

TABLE 1
Overview of bispecific antibody components
and corresponding VH/VL SEQ ID NOs

| | Component anti-FIX | | Component anti-FX | |
|---|---|---|---|---|
| | antibody | | antibody | |
| BimAb ID | ID | # VH\|VL | ID | # VH\|VL |
| bimAb05-0745 | mAb01-9994 | 67\|71 | mAb01-8174 | 467\|471 |
| bimAb05-0746 | mAb01-9978 | 43\|47 | mAb01-8174 | 467\|471 |
| bimAb05-1229 | mAb01-9016 | 3\|7 | mAb01-6723 | 459\|463 |
| bimAb05-2112 | mAb01-9994 | 67\|71 | mAb01-9772 | 483\|487 |
| bimAb05-2113 | mAb01-9933 | 35\|39 | mAb01-9772 | 483\|487 |
| bimAb05-2114 | mAb01-9985 | 51\|55 | mAb01-9772 | 483\|487 |
| bimAb05-2115 | mAb01-9978 | 43\|47 | mAb01-9772 | 483\|487 |
| bimAb05-2375 | mAb01-9016 | 3\|7 | mAb01-8174 | 467\|471 |
| bimAb05-2379 | mAb01-9016 | 3\|7 | mAb01-8913 | 475\|479 |
| bimAb05-2532 | mAb01-9373 | 11\|15 | mAb01-6723 | 459\|463 |
| bimAb05-3279 | mAb01-9933 | 35\|39 | mAb01-6723 | 459\|463 |
| bimAb05-3409 | mAb01-9978 | 43\|47 | mAb01-6723 | 459\|463 |
| bimAb05-3416 | mAb01-9985 | 51\|55 | mAb01-6723 | 459\|463 |
| bimAb05-3755 | mAb01-9994 | 67\|71 | mAb01-6723 | 459\|463 |
| bimAb05-3761 | mAb01-9933 | 35\|39 | mAb01-8174 | 467\|471 |
| bimAb05-3769 | mAb01-9985 | 51\|55 | mAb01-8174 | 467\|471 |
| bimAb05-3770 | mAb01-9986 | 59\|63 | mAb01-8174 | 467\|471 |
| bimAb05-3862 | mAb01-9696 | 19\|23 | mAb01-8174 | 467\|471 |
| bimAb05-3863 | mAb01-9697 | 27\|31 | mAb01-8174 | 467\|471 |
| bimAb05-3880 | mAb11-0047 | 75\|79 | mAb01-8174 | 467\|471 |
| bimAb05-3886 | mAb11-0049 | 83\|87 | mAb01-8174 | 467\|471 |
| bimAb05-3955 | mAb11-0107 | 91\|95 | mAb01-8174 | 467\|471 |
| bimAb05-4100 | mAb11-0149 | 99\|103 | mAb01-8174 | 467\|471 |
| bimAb05-4114 | mAb11-0160 | 107\|111 | mAb01-8174 | 467\|471 |
| bimAb05-4121 | mAb11-0164 | 115\|119 | mAb01-8174 | 467\|471 |
| bimAb05-4220 | mAb11-0962 | 211\|215 | mAb01-8174 | 467\|471 |
| bimAb05-4226 | mAb11-0965 | 219\|223 | mAb01-8174 | 467\|471 |
| bimAb05-4283 | mAb11-1021 | 227\|231 | mAb01-8174 | 467\|471 |
| bimAb05-4289 | mAb11-1024 | 235\|239 | mAb01-8174 | 467\|471 |
| bimAb05-4292 | mAb11-1033 | 251\|255 | mAb01-8174 | 467\|471 |
| bimAb05-4293 | mAb11-1030 | 243\|247 | mAb01-8174 | 467\|471 |
| bimAb05-4387 | mAb11-1039 | 259\|263 | mAb01-8174 | 467\|471 |
| bimAb05-4392 | mAb11-1042 | 267\|271 | mAb01-8174 | 467\|471 |
| bimAb05-4419 | mAb11-0174 | 123\|127 | mAb01-8174 | 467\|471 |
| bimAb05-4422 | mAb11-1073 | 275\|279 | mAb01-8174 | 467\|471 |
| bimAb05-4428 | mAb11-1076 | 283\|287 | mAb01-8174 | 467\|471 |
| bimAb05-4443 | mAb11-1094 | 299\|303 | mAb01-8174 | 467\|471 |
| bimAb05-4444 | mAb11-1091 | 291\|295 | mAb01-8174 | 467\|471 |
| bimAb05-4601 | mAb11-0937 | 187\|191 | mAb01-8174 | 467\|471 |
| bimAb05-4604 | mAb11-0923 | 139\|143 | mAb01-8174 | 467\|471 |
| bimAb05-4608 | mAb11-0939 | 203\|207 | mAb01-8174 | 467\|471 |
| bimAb05-4611 | mAb11-0935 | 179\|183 | mAb01-8174 | 467\|471 |
| bimAb05-4612 | mAb11-0938 | 195\|199 | mAb01-8174 | 467\|471 |
| bimAb05-4613 | mAb11-0933 | 171\|175 | mAb01-8174 | 467\|471 |
| bimAb05-4615 | mAb11-0932 | 163\|167 | mAb01-8174 | 467\|471 |
| bimAb05-4617 | mAb11-0929 | 147\|151 | mAb01-8174 | 467\|471 |
| bimAb05-4618 | mAb11-0931 | 155\|159 | mAb01-8174 | 467\|471 |
| bimAb05-4684 | mAb01-9985 | 51\|55 | mAb11-1114 | 499\|503 |
| bimAb05-4685 | mAb01-9985 | 51\|55 | mAb11-1115 | 507\|511 |
| bimAb05-4686 | mAb01-9985 | 51\|55 | mAb11-1116 | 515\|519 |
| bimAb05-4687 | mAb01-9985 | 51\|55 | mAb11-1117 | 523\|527 |
| bimAb05-4688 | mAb01-9985 | 51\|55 | mAb11-1118 | 531\|535 |
| bimAb05-4689 | mAb01-9985 | 51\|55 | mAb11-1119 | 539\|543 |
| bimAb05-4690 | mAb01-9985 | 51\|55 | mAb11-1121 | 555\|559 |
| bimAb05-4692 | mAb01-9985 | 51\|55 | mAb11-1122 | 563\|567 |
| bimAb05-4693 | mAb01-9985 | 51\|55 | mAb11-1123 | 571\|575 |
| bimAb05-4694 | mAb01-9985 | 51\|55 | mAb11-1124 | 579\|583 |
| bimAb05-4695 | mAb01-9985 | 51\|55 | mAb01-9778 | 491\|495 |
| bimAb05-4696 | mAb01-9985 | 51\|55 | mAb11-1125 | 587\|591 |
| bimAb05-4697 | mAb01-9985 | 51\|55 | mAb11-1127 | 595\|599 |
| bimAb05-4698 | mAb01-9985 | 51\|55 | mAb11-1128 | 603\|607 |
| bimAb05-4699 | mAb01-9985 | 51\|55 | mAb11-1129 | 611\|615 |
| bimAb05-4700 | mAb01-9985 | 51\|55 | mAb11-1130 | 619\|623 |
| bimAb05-4701 | mAb01-9985 | 51\|55 | mAb11-1131 | 627\|631 |
| bimAb05-4702 | mAb01-9985 | 51\|55 | mAb11-1132 | 635\|639 |
| bimAb05-4703 | mAb01-9985 | 51\|55 | mAb11-1133 | 643\|647 |
| bimAb05-4704 | mAb01-9985 | 51\|55 | mAb11-1416 | 651\|655 |
| bimAb05-4705 | mAb01-9985 | 51\|55 | mAb11-1417 | 659\|663 |
| bimAb05-4706 | mAb01-9985 | 51\|55 | mAb11-1418 | 667\|671 |
| bimAb05-4707 | mAb01-9985 | 51\|55 | mAb11-1419 | 675\|679 |
| bimAb05-4708 | mAb01-9985 | 51\|55 | mAb11-1420 | 683\|687 |
| bimAb05-4709 | mAb01-9985 | 51\|55 | mAb11-1421 | 691\|695 |
| bimAb05-4710 | mAb01-9985 | 51\|55 | mAb11-1422 | 699\|703 |
| bimAb05-4788 | mAb11-1233 | 307\|311 | mAb01-8174 | 467\|471 |
| bimAb05-4884 | mAb11-1254 | 315\|319 | mAb01-8174 | 467\|471 |
| bimAb05-4895 | mAb11-1262 | 339\|343 | mAb01-8174 | 467\|471 |
| bimAb05-4896 | mAb11-1259 | 323\|327 | mAb01-8174 | 467\|471 |
| bimAb05-4898 | mAb11-1260 | 331\|335 | mAb01-8174 | 467\|471 |
| bimAb05-4903 | mAb11-1268 | 355\|359 | mAb01-8174 | 467\|471 |
| bimAb05-4906 | mAb11-1266 | 347\|351 | mAb01-8174 | 467\|471 |
| bimAb05-4910 | mAb11-1273 | 363\|367 | mAb01-8174 | 467\|471 |
| bimAb05-4914 | mAb11-1275 | 371\|375 | mAb01-8174 | 467\|471 |
| bimAb05-4915 | mAb11-1276 | 379\|383 | mAb01-8174 | 467\|471 |
| bimAb05-4919 | mAb11-1278 | 387\|391 | mAb01-8174 | 467\|471 |
| bimAb05-4920 | mAb11-1282 | 403\|407 | mAb01-8174 | 467\|471 |
| bimAb05-4921 | mAb11-1279 | 395\|399 | mAb01-8174 | 467\|471 |
| bimAb05-4924 | mAb11-1288 | 419\|423 | mAb01-8174 | 467\|471 |
| bimAb05-4927 | mAb11-1286 | 411\|415 | mAb01-8174 | 467\|471 |
| bimAb05-5092 | mAb11-1344 | 427\|431 | mAb01-8174 | 467\|471 |
| bimAb05-5095 | mAb11-0723 | 131\|135 | mAb01-8174 | 467\|471 |
| bimAb05-5204 | mAb11-1358 | 435\|439 | mAb01-8174 | 467\|471 |
| bimAb05-5205 | mAb11-1360 | 443\|447 | mAb01-8174 | 467\|471 |
| bimAb05-5240 | mAb11-1389 | 451\|455 | mAb01-8174 | 467\|471 |
| bimAb05-5339 | mAb01-9985 | 51\|55 | mAb11-1440 | 771\|775 |
| bimAb05-5340 | mAb01-9985 | 51\|55 | mAb11-1431 | 707\|711 |
| bimAb05-5341 | mAb01-9985 | 51\|55 | mAb11-1441 | 779\|783 |
| bimAb05-5342 | mAb01-9985 | 51\|55 | mAb11-1439 | 763\|767 |
| bimAb05-5343 | mAb01-9985 | 51\|55 | mAb11-1442 | 787\|791 |
| bimAb05-5344 | mAb01-9985 | 51\|55 | mAb11-1443 | 795\|799 |
| bimAb05-5345 | mAb01-9985 | 51\|55 | mAb11-1446 | 819\|823 |
| bimAb05-5346 | mAb01-9985 | 51\|55 | mAb11-1444 | 803\|807 |
| bimAb05-5347 | mAb01-9985 | 51\|55 | mAb11-1447 | 827\|831 |
| bimAb05-5348 | mAb01-9985 | 51\|55 | mAb11-1445 | 811\|815 |
| bimAb05-5349 | mAb01-9985 | 51\|55 | mAb11-1448 | 835\|839 |
| bimAb05-5350 | mAb01-9985 | 51\|55 | mAb11-1432 | 715\|719 |
| bimAb05-5351 | mAb01-9985 | 51\|55 | mAb11-1449 | 843\|847 |
| bimAb05-5352 | mAb01-9985 | 51\|55 | mAb11-1450 | 851\|855 |
| bimAb05-5353 | mAb01-9985 | 51\|55 | mAb11-1453 | 875\|879 |
| bimAb05-5354 | mAb01-9985 | 51\|55 | mAb11-1451 | 859\|863 |
| bimAb05-5355 | mAb01-9985 | 51\|55 | mAb11-1454 | 883\|887 |
| bimAb05-5356 | mAb01-9985 | 51\|55 | mAb11-1452 | 867\|871 |
| bimAb05-5357 | mAb01-9985 | 51\|55 | mAb11-1455 | 891\|895 |
| bimAb05-5358 | mAb01-9985 | 51\|55 | mAb11-1433 | 723\|727 |
| bimAb05-5359 | mAb01-9985 | 51\|55 | mAb11-1456 | 899\|903 |
| bimAb05-5361 | mAb01-9985 | 51\|55 | mAb11-1459 | 923\|927 |
| bimAb05-5362 | mAb01-9985 | 51\|55 | mAb11-1457 | 907\|911 |
| bimAb05-5363 | mAb01-9985 | 51\|55 | mAb11-1434 | 731\|735 |
| bimAb05-5364 | mAb01-9985 | 51\|55 | mAb11-1458 | 915\|919 |
| bimAb05-5365 | mAb01-9985 | 51\|55 | mAb11-1460 | 931\|935 |
| bimAb05-5366 | mAb01-9985 | 51\|55 | mAb11-1461 | 939\|943 |
| bimAb05-5367 | mAb01-9985 | 51\|55 | mAb11-1465 | 963\|967 |
| bimAb05-5369 | mAb01-9985 | 51\|55 | mAb11-1466 | 971\|975 |
| bimAb05-5370 | mAb01-9985 | 51\|55 | mAb11-1463 | 947\|951 |
| bimAb05-5371 | mAb01-9985 | 51\|55 | mAb11-1435 | 739\|743 |
| bimAb05-5372 | mAb01-9985 | 51\|55 | mAb11-1464 | 955\|959 |
| bimAb05-5373 | mAb01-9985 | 51\|55 | mAb11-1467 | 979\|983 |
| bimAb05-5374 | mAb01-9985 | 51\|55 | mAb11-1468 | 987\|991 |
| bimAb05-5375 | mAb01-9985 | 51\|55 | mAb11-1472 | 1011\|1015 |
| bimAb05-5377 | mAb01-9985 | 51\|55 | mAb11-1473 | 1019\|1023 |
| bimAb05-5378 | mAb01-9985 | 51\|55 | mAb11-1470 | 995\|999 |
| bimAb05-5379 | mAb01-9985 | 51\|55 | mAb11-1436 | 747\|751 |
| bimAb05-5380 | mAb01-9985 | 51\|55 | mAb11-1471 | 1003\|1007 |
| bimAb05-5381 | mAb01-9985 | 51\|55 | mAb11-1474 | 1027\|1031 |
| bimAb05-5383 | mAb01-9985 | 51\|55 | mAb11-1477 | 1051\|1055 |
| bimAb05-5384 | mAb01-9985 | 51\|55 | mAb11-1475 | 1035\|1039 |
| bimAb05-5385 | mAb01-9985 | 51\|55 | mAb11-1478 | 1059\|1063 |
| bimAb05-5386 | mAb01-9985 | 51\|55 | mAb11-1476 | 1043\|1047 |
| bimAb05-5387 | mAb01-9985 | 51\|55 | mAb11-1479 | 1067\|1071 |
| bimAb05-5388 | mAb01-9985 | 51\|55 | mAb11-1480 | 1075\|1079 |
| bimAb05-5389 | mAb01-9985 | 51\|55 | mAb11-1483 | 1099\|1103 |
| bimAb05-5390 | mAb01-9985 | 51\|55 | mAb11-1437 | 755\|759 |
| bimAb05-5391 | mAb01-9985 | 51\|55 | mAb11-1484 | 1107\|1111 |
| bimAb05-5392 | mAb01-9985 | 51\|55 | mAb11-1481 | 1083\|1087 |
| bimAb05-5393 | mAb01-9985 | 51\|55 | mAb11-1485 | 1115\|1119 |
| bimAb05-5394 | mAb01-9985 | 51\|55 | mAb11-1482 | 1091\|1095 |
| bimAb05-5395 | mAb01-9985 | 51\|55 | mAb11-1486 | 1123\|1127 |
| bimAb05-5396 | mAb01-9985 | 51\|55 | mAb11-1487 | 1131\|1135 |
| bimAb05-5397 | mAb01-9985 | 51\|55 | mAb11-1490 | 1155\|1159 |
| bimAb05-5399 | mAb01-9985 | 51\|55 | mAb11-1491 | 1163\|1167 |
| bimAb05-5400 | mAb01-9985 | 51\|55 | mAb11-1488 | 1139\|1143 |
| bimAb05-5401 | mAb01-9985 | 51\|55 | mAb11-1492 | 1171\|1175 |
| bimAb05-5402 | mAb01-9985 | 51\|55 | mAb11-1489 | 1147\|1151 |
| bimAb05-5403 | mAb01-9985 | 51\|55 | mAb11-1493 | 1179\|1183 |
| bimAb05-5406 | mAb01-9985 | 51\|55 | mAb11-1494 | 1187\|1191 |
| bimAb05-5413 | mAb01-9985 | 51\|55 | mAb11-1120 | 547\|551 |
| bimAb05-4271 | mAb11-0173 | 1202\|1206 | mAb01-8174 | 467\|471 |
| bimAb05-4756 | mAb11-1204 | 1210\|1214 | mAb01-8174 | 467\|471 |
| bimAb05-0396 | mAb11-1495 | 1218\|1222 | mAb01-8174 | 467\|471 |
| bimAb05-0417 | mAb11-1501 | 1226\|1230 | mAb01-8174 | 467\|471 |
| bimAb05-0438 | mAb11-1502 | 1234\|1238 | mAb01-8174 | 467\|471 |

TABLE 2
Overview of anti-FIX(a) antibody VH, VL and CDR sequences

| | VH/VL | | | CDR1 | | CDR2 | | CDR3 | |
|---|---|---|---|---|---|---|---|---|---|
| mAb ID | Domain | Sequence | # | Sequence | # | Sequence | # | Sequence | # |
| mAb01- | VH | EVQLVESGGGLVQPGRS | 3 | DYAMH | 4 | GISWRGDIGGYV | 5 | SYGSGSFYNAFDS | 6 |
| 9016 | | LKLSCAASGFTFDDYAMH | | | | DSVKG | | | |
| | | WVRQVPGKGLEWVSGIS | | | | | | | |
| | | WRGDIGGYVDSVKGRFTI | | | | | | | |
| | | SRDNAKNSLYLQLNSLRA | | | | | | | |
| | | EDTALYYCVKSYGSGSFY | | | | | | | |
| | | NAFDSWGQGTLVTVSS | | | | | | | |
| mAb01- | VL | DIQMTQSPSTLSASAGDR | 7 | RASQSISSW | 8 | KASRLER | 9 | LEYSSYIRT | 10 |
| 9016 | | VTITCRASQSISSWLAWY | | LA | | | | | |
| | | QQKPGKAPKFLIYKASRL | | | | | | | |
| | | ERGVPSRFSGSGSGTEF | | | | | | | |
| | | SLTISSLQPDDFATYYCLE | | | | | | | |
| | | YSSYIRTFGQGTKVEIK | | | | | | | |
| mAb01- | VH | EVQLVESGGGLVQPGRS | 11 | DYAMH | 12 | GISWRGDIIGYVD | 13 | SYGSGSFYNAFDS | 14 |
| 9373 | | LKLSCAASGFTFHDYAMH | | | | SVKG | | | |
| | | WVRQVPGKGLEWVSGIS | | | | | | | |
| | | WRGDIIGYVDSVKGRFTIS | | | | | | | |
| | | RDNAKNSLYLQLNSLRAE | | | | | | | |
| | | DTALYYCVKSYGSGSFYN | | | | | | | |
| | | AFDSWGQGTLVTVSS | | | | | | | |
| mAb01- | VL | DIQMTQSPSTLSASAGDE | 15 | RASKSISSW | 16 | KASRLDR | 17 | LEYSSYIRT | 18 |
| 9373 | | VTITCRASKSISSWLAWY | | LA | | | | | |
| | | QQKPGKAPRFLIYKASRL | | | | | | | |
| | | DRGTPSRFSGSGSGTEF | | | | | | | |
| | | TLTISHLQPDDFATYYCLE | | | | | | | |
| | | YSSYIRTFGQGTKVEIK | | | | | | | |
| mAb01- | VH | EVQLVESGGGLVQPGRS | 19 | DYAMH | 20 | GISWRGDIGGYV | 21 | SYGSGSFYNAFDS | 22 |
| 9696 | | LKLSCAASGFTFDDYAMH | | | | DSVKG | | | |
| | | WVRQVPGKGLEWVSGIS | | | | | | | |
| | | WRGDIGGYVDSVKGRFTI | | | | | | | |
| | | SRDNAKNSLYLQLNSLRA | | | | | | | |
| | | EDTALYYCVKSYGSGSFY | | | | | | | |
| | | NAFDSWGQGTLVTVSS | | | | | | | |
| mAb01- | VL | DIQMTQSPSTLSASAGDR | 23 | RASQSISSW | 24 | KASRLDR | 25 | LEYSSYIRT | 26 |
| 9696 | | VTITCRASQSISSWLAWY | | LA | | | | | |
| | | QQKPGKAPKFLIYKASRL | | | | | | | |
| | | DRGTPSRFSGSGSGTEF | | | | | | | |
| | | SLTISSLQPDDFATYYCLE | | | | | | | |
| | | YSSYIRTFGQGTKVEIK | | | | | | | |
| mAb01- | VH | EVQLVESGGGLVQPGRS | 27 | DYAMH | 28 | GISWRGDIGGYV | 29 | SYGSGSFYNAFDS | 30 |
| 9697 | | LKLSCAASGFTFDDYAMH | | | | DSVKG | | | |
| | | WVRQVPGKGLEWVSGIS | | | | | | | |
| | | WRGDIGGYVDSVKGRFTI | | | | | | | |
| | | SRDNAKNSLYLQLNSLRA | | | | | | | |
| | | EDTALYYCVKSYGSGSFY | | | | | | | |
| | | NAFDSWGQGTLVTVSS | | | | | | | |
| mAb01- | VL | DIQMTQSPSTLSASVGDR | 31 | RASQSISSW | 32 | KASRLER | 33 | LEYSSYIRT | 34 |
| 9697 | | VTITCRASQSISSWLAWY | | LA | | | | | |
| | | QQKPGKAPKFLIYKASRL | | | | | | | |
| | | ERGTPSRFSGSGSGTEF | | | | | | | |
| | | SLTISSLQPDDFATYYCLE | | | | | | | |
| | | YSSYIRTFGQGTKVEIK | | | | | | | |
| mAb01- | VH | EVQLVESGGGLVQPGRS | 35 | DYAMH | 36 | GISWKGDIGGYV | 37 | SYGSGSFYNAFDS | 38 |
| 9933 | | LKLSCAASGFTFDDYAMH | | | | DSVKG | | | |
| | | WVRQVPGKGLEWVSGIS | | | | | | | |
| | | WKGDIGGYVDSVKGRFTI | | | | | | | |
| | | SRDNAKNSLYLQLNSLRA | | | | | | | |
| | | EDTALYYCAKSYGSGSFY | | | | | | | |
| | | NAFDSWGQGTLVTVSS | | | | | | | |
| mAb01- | VL | DIQMTQSPSTLSASAGDE | 39 | RASQSISSW | 40 | KASKLDR | 41 | LEYSSYIRT | 42 |
| 9933 | | VTITCRASQSISSWLAWY | | LA | | | | | |
| | | QQKPGKAPKLLIYKASKL | | | | | | | |
| | | DRGVPSRFSGSGSGTEF | | | | | | | |
| | | SLTISDLQPDDFATYYCLE | | | | | | | |
| | | YSSYIRTFGQGTKVEIK | | | | | | | |
| mAb01- | VH | EVQLVESGGGLVQPGRS | 43 | DYAMH | 44 | GISWKGDIGGYV | 45 | SYGSGSFYNAFDS | 46 |
| 9978 | | LRLSCAASGFTFHDYAMH | | | | DSVKG | | | |
| | | WVRQVPGKGLEWVSGIS | | | | | | | |
| | | WKGDIGGYVDSVKGRFTI | | | | | | | |
| | | SRDNEKNSLYLQMNSLR | | | | | | | |
| | | AEDTALYYCVKSYGSGSF | | | | | | | |
| | | YNAFDSWGQGTLVTVSS | | | | | | | |
| mAb01- | VL | DIQMTQSPSTLSASVGDR | 47 | RASQSISSW | 48 | KASRLER | 49 | LEYSSYIRT | 50 |
| 9978 | | VTITCRASQSISSWLAWY | | LA | | | | | |
| | | QQKPGKAPKLLIYKASRL | | | | | | | |
| | | ERGTPSRFSGSGSGTEF | | | | | | | |
| | | SLTISSLQPDDFATYYCLE | | | | | | | |
| | | YSSYIRTFGQGTKVEIK | | | | | | | |
| mAb01- | VH | EVQLVESGGGLVQPGRS | 51 | DYAMH | 52 | GISWRGDIGGYV | 53 | SYGSGSFYNAFDS | 54 |
| 9985 | | LRLSCAASGFTFHDYAMH | | | | KSVKG | | | |
| | | WVRQVPGKGLEWVSGIS | | | | | | | |
| | | WRGDIGGYVKSVKGRFTI | | | | | | | |
| | | SRDNAKNSLYLQMNSLR | | | | | | | |
| | | AEDTALYYCVKSYGSGSF | | | | | | | |
| | | YNAFDSWGQGTLVTVSS | | | | | | | |
| mAb01- | VL | DIQMTQSPSTLSASVGDR | 55 | RASQSISSW | 56 | KASKLER | 57 | LEYSSYIRT | 58 |
| 9985 | | VTITCRASQSISSWLAWY | | LA | | | | | |
| | | QQKPGKAPKFLIYKASKL | | | | | | | |
| | | ERGTPSRFSGSGSGTEF | | | | | | | |
| | | SLTISSLQPDDFATYYCLE | | | | | | | |
| | | YSSYIRTFGQGTKVEIK | | | | | | | |
| mAb01- | VH | EVQLVESGGGLVQPGRS | 59 | DYAMH | 60 | GISWRGDIGGYV | 61 | SYGSGSFYNAFDS | 62 |
| 9986 | | LRLSCAASGFTFHDYAMH | | | | KSVKG | | | |
| | | WVRQVPGKGLEWVSGIS | | | | | | | |
| | | WRGDIGGYVKSVKGRFTI | | | | | | | |
| | | SRDNAKNSLYLQMNSLR | | | | | | | |
| | | AEDTALYYCVKSYGSGSF | | | | | | | |
| | | YNAFDSWGQGTLVTVSS | | | | | | | |
| mAb01- | VL | DIQMTQSPSTLSASVGDR | 63 | RASQSISSW | 64 | KASRLER | 65 | LEYSSYIRT | 66 |
| 9986 | | VTITCRASQSISSWLAWY | | LA | | | | | |
| | | QQKPGKAPKFLIYKASRL | | | | | | | |
| | | ERGTPSRFSGSGSGTEF | | | | | | | |
| | | SLTISSLQPDDFATYYCLE | | | | | | | |
| | | YSSYIRTFGQGTKVEIK | | | | | | | |
| mAb01- | VH | EVQLVESGGGLVQPGRS | 67 | DYAMH | 68 | GISWKGDIGGYV | 69 | SYGSGSFYNAFDS | 70 |
| 9994 | | LRLSCAASGFTFHDYAMH | | | | DSVKG | | | |
| | | WVRQVPGKGLEWVSGIS | | | | | | | |
| | | WKGDIGGYVDSVKGRFTI | | | | | | | |
| | | SRDNEKNSLYLQMNSLR | | | | | | | |
| | | AEDTALYYCVKSYGSGSF | | | | | | | |
| | | YNAFDSWGQGTLVTVSS | | | | | | | |
| mAb01- | VL | DIQMTQSPSTLSASVGDR | 71 | RASQSISSW | 72 | KASRLDR | 73 | LEYSSYIRT | 74 |
| 9994 | | VTITCRASQSISSWLAWY | | LA | | | | | |
| | | QQKPGKAPKFLIYKASRL | | | | | | | |
| | | DRGTPSRFSGSGSGTEF | | | | | | | |
| | | SLTISSLQPDDFATYYCLE | | | | | | | |
| | | YSSYIRTFGQGTKVEIK | | | | | | | |
| mAb11- | VH | EVQLVESGGGLVQPGRS | 75 | DYAMH | 76 | GISWRGDIKGYVD | 77 | SYGSGSFYNAFDS | 78 |
| 0047 | | LRLSCAASGFTFHDYAMH | | | | SVKG | | | |
| | | WVRQAPGKGLEWVSGIS | | | | | | | |
| | | WRGDIKGYVDSVKGRFTI | | | | | | | |
| | | SRDNAKNSLYLQLNSLRA | | | | | | | |
| | | EDTALYYCAKSYGSGSFY | | | | | | | |
| | | NAFDSWGQGTLVTVSS | | | | | | | |
| mAb11- | VL | DIQMTQSPSTLSASAGDR | 79 | RASKSISSW | 80 | KASRLDR | 81 | LEYSSYIRT | 82 |
| 0047 | | VTITCRASKSISSWLAWY | | LA | | | | | |
| | | QQKPGKAPKFLIYKASRL | | | | | | | |
| | | DRGTPQRFSGSGSGTEF | | | | | | | |
| | | SLTISSLQPDDFATYYCLE | | | | | | | |
| | | YSSYIRTFGQGTKVEIK | | | | | | | |
| mAb11- | VH | EVQLVESGGGLVQPGRS | 83 | DYAMH | 84 | GISWRGDIKGYVD | 85 | SYGSGSFYNAFDS | 86 |
| 0049 | | LRLSCAASGFTFHDYAMH | | | | SVKG | | | |
| | | WVRQAPGKGLEWVSGIS | | | | | | | |
| | | WRGDIKGYVDSVKGRFTI | | | | | | | |
| | | SRDNAKNSLYLQLNSLRA | | | | | | | |
| | | EDTALYYCAKSYGSGSFY | | | | | | | |
| | | NAFDSWGQGTLVTVSS | | | | | | | |
| mAb11- | VL | DIQMTQSPSTLSASAGDR | 87 | RASQSISSW | 88 | KASRLDR | 89 | LEYNSYIRT | 90 |
| 0049 | | VTISCRASQSISSWLAWY | | LA | | | | | |
| | | QQKPGKAPKLLIYKASRL | | | | | | | |
| | | DRGTPSRFSGSGSGTEF | | | | | | | |
| | | TLTISSLQPDDFATYYCLE | | | | | | | |
| | | YNSYIRTFGQGTKVEIK | | | | | | | |
| mAb11- | VH | EVQLVESGGGLVQPGRS | 91 | DYAMH | 92 | GISWRGDIGGYV | 93 | SYGSGSFYNAFDS | 94 |
| 0107 | | LKLSCAASGFTFHDYAMH | | | | KSVKG | | | |
| | | WVRQVPGKGLEWVSGIS | | | | | | | |
| | | WRGDIGGYVKSVKGRFTI | | | | | | | |
| | | SRDNAKNSLYLQLNSLRA | | | | | | | |
| | | EDTALYYCVKSYGSGSFY | | | | | | | |
| | | NAFDSWGQGTLVTVSS | | | | | | | |
| mAb11- | VL | DIQMTQSPSTLSASVGDE | 95 | RASQSISSW | 96 | KAQRLDR | 97 | LEYSSYIRT | 98 |
| 0107 | | VTITCRASQSISSWLAWY | | LA | | | | | |
| | | QQKPGKAPKFLIYKAQRL | | | | | | | |
| | | DRGTPQRFSGSGSGTEF | | | | | | | |
| | | TLTISSLQPDDFATYYCLE | | | | | | | |
| | | YSSYIRTFGQGTKVEIK | | | | | | | |
| mAb11- | VH | EVQLVESGGGLVQPGRS | 99 | DYAMH | 100 | GISWKGDIGGYV | 101 | SYGSGSFYNAFDS | 102 |
| 0149 | | LKLSCAASGFTFDDYAMH | | | | DSVKG | | | |
| | | WVRQAPGKGLEWVSGIS | | | | | | | |
| | | WKGDIGGYVDSVKGRFTI | | | | | | | |
| | | SRDNAKNSLYLQMNSLR | | | | | | | |
| | | AEDTALYYCVKSYGSGSF | | | | | | | |
| | | YNAFDSWGQGTLVTVSS | | | | | | | |
| mAb11- | VL | DIQMTQSPSTLSASAGDE | 103 | RASQSIKSW | 104 | KASRLDR | 105 | LEYSSYIRT | 106 |
| 0149 | | VTITCRASQSIKSWLAWY | | LA | | | | | |
| | | QQKPGKAPKFLIYKASRL | | | | | | | |
| | | DRGTPQRFSGSGSGTEF | | | | | | | |
| | | SLTISRLQPDDFATYYCLE | | | | | | | |
| | | YSSYIRTFGQGTKVEIK | | | | | | | |
| mAb11- | VH | EVQLVESGGGLVQPGRS | 107 | DYAMH | 108 | GISWKGDIGGYA | 109 | SYGSGSFYNAFDS | 110 |
| 0160 | | LKLSCAASGFRFDDYAMH | | | | DSVKG | | | |
| | | WVRQVPGKGLEWVSGIS | | | | | | | |
| | | WKGDIGGYADSVKGRFTI | | | | | | | |
| | | SRDNAKNSLYLQLNSLRA | | | | | | | |
| | | EDTALYYCVKSYGSGSFY | | | | | | | |
| | | NAFDSWGQGTLVTVSS | | | | | | | |
| mAb11- | VL | DIQMTQSPSTLSASVGDE | 111 | RASKSISSW | 112 | KASRLER | 113 | LEYSSYIRT | 114 |
| 0160 | | VTITCRASKSISSWLAWY | | LA | | | | | |
| | | QQKPGKAPRFLIYKASRL | | | | | | | |
| | | ERGTPQRFSGSGSGTEF | | | | | | | |
| | | TLTIYSLQPDDFATYYCLE | | | | | | | |
| | | YSSYIRTFGQGTKVEIK | | | | | | | |
| mAb11- | VH | EVQLVESGGGLVQPGRS | 115 | DYAMH | 116 | GISWKGDIGGYA | 117 | SYGSGSFYNAFDS | 118 |
| 0164 | | LKLSCAASGFRFDDYAMH | | | | DSVKG | | | |
| | | WVRQVPGKGLEWVSGIS | | | | | | | |
| | | WKGDIGGYADSVKGRFTI | | | | | | | |
| | | SRDNAKNSLYLQLNSLRA | | | | | | | |
| | | EDTALYYCVKSYGSGSFY | | | | | | | |
| | | NAFDSWGQGTLVTVSS | | | | | | | |
| mAb11- | VL | DIQMTQSPSTLSASVGDR | 119 | RASQSISSW | 120 | KASRLER | 121 | LEYSSYIRT | 122 |
| 0164 | | VTITCRASQSISSWLAWY | | LA | | | | | |
| | | QQKPGKAPKFLIYKASRL | | | | | | | |
| | | ERGTPSRFSGSGSGTEF | | | | | | | |
| | | SLTISSLQPDDFATYYCLE | | | | | | | |
| | | YSSYIRTFGQGTKVEIK | | | | | | | |
| mAb11- | VH | EVQLVESGGGLVQPGRS | 123 | DYAMH | 124 | GISWKGDIGGYV | 125 | SYGSGSFYNAFDS | 126 |
| 0174 | | LKLSCAASGFTFHDYAMH | | | | DSVKG | | | |
| | | WVRQAPGKGLEWVSGIS | | | | | | | |
| | | WKGDIGGYVDSVKGRFTI | | | | | | | |
| | | SRDNAKNSLYLQMNSLR | | | | | | | |
| | | AEDTALYYCAKSYGSGSF | | | | | | | |
| | | YNAFDSWGQGTLVTVSS | | | | | | | |
| mAb11- | VL | DIQMTQSPSTLSASVGDR | 127 | RASQSISSW | 128 | KASKLDR | 129 | LEYSSYIRT | 130 |
| 0174 | | VTITCRASQSISSWLAWY | | LA | | | | | |
| | | QQKPGKAPKFLIYKASKL | | | | | | | |
| | | DRGTPSRFSGSGSGTEF | | | | | | | |
| | | TLTISSLQPDDFATYYCLE | | | | | | | |
| | | YSSYIRTFGQGTKVEIK | | | | | | | |
| mAb11- | VH | EVQLVESGGGLVQPGRS | 131 | DYAMH | 132 | GISWKGDIGGYV | 133 | SYGSGSFYNAFDS | 134 |
| 0723 | | LKLSCAASGFTFDDYAMH | | | | DSVKG | | | |
| | | WVRQVPGKGLEWVSGIS | | | | | | | |
| | | WKGDIGGYVDSVKGRFTI | | | | | | | |
| | | SRDNAKNSLYLQLNSLRA | | | | | | | |
| | | EDTALYYCAKSYGSGSFY | | | | | | | |
| | | NAFDSWGQGTLVTVSS | | | | | | | |
| mAb11- | VL | DIQMTQSPSTLSASAGDR | 135 | RASQSISSW | 136 | KASRLDR | 137 | LEYSSYIRT | 138 |
| 0723 | | VTITCRASQSISSWLAWY | | LA | | | | | |
| | | QQKPGKAPKFLIYKASRL | | | | | | | |
| | | DRGTPSRFSGSGSGTEF | | | | | | | |
| | | SLTISSLQPDDFATYYCLE | | | | | | | |
| | | YSSYIRTFGQGTKVEIK | | | | | | | |
| mAb11- | VH | EVQLVESGGGLVQPGRS | 139 | DYAMH | 140 | GISWKGDIGGYV | 141 | SYGSGSFYNAFDS | 142 |
| 0923 | | LRLSCAASGFTFHDYAMH | | | | DSVKG | | | |
| | | WVRQVPGKGLEWVSGIS | | | | | | | |
| | | WKGDIGGYVDSVKGRFTI | | | | | | | |
| | | SRDNEKNSLYLQMNSLR | | | | | | | |
| | | AEDTALYYCVKSYGSGSF | | | | | | | |
| | | YNAFDSWGQGTLVTVSS | | | | | | | |
| mAb11- | VL | DIQMTQSPSTLSASVGDR | 143 | RASQSISSW | 144 | KASRLDR | 145 | LEYSSYIRT | 146 |
| 0923 | | VTITCRASQSISSWLAWY | | LA | | | | | |
| | | QQKPGKAPKFLIYKASRL | | | | | | | |
| | | DRGCPSRFSGSGSGTEF | | | | | | | |
| | | SLTISSLQPDDFATYYCLE | | | | | | | |
| | | YSSYIRTFGQGTKVEIK | | | | | | | |
| mAb11- | VH | EVQLVESGGGLVQPGRS | 147 | DYAMH | 148 | GISWKGDIGGYV | 149 | SYGSGSFYNAFDS | 150 |
| 0929 | | LRLSCAASGFTFHDYAMH | | | | DSVKG | | | |
| | | WVRQVPGKGLEWVSGIS | | | | | | | |
| | | WKGDIGGYVDSVKGRFTI | | | | | | | |
| | | SRDNEKNSLYLQMNSLR | | | | | | | |
| | | AEDTALYYCVKSYGSGSF | | | | | | | |
| | | YNAFDSWGQGTLVTVSS | | | | | | | |
| mAb11- | VL | DIQMTQSPSTLSASVGDR | 151 | RASQSISSW | 152 | KASRLDR | 153 | LEYSSYIRT | 154 |
| 0929 | | VTITCRASQSISSWLAWY | | LA | | | | | |
| | | QQKPGKAPKFLIYKASRL | | | | | | | |
| | | DRGIPSRFSGSGSGTEFS | | | | | | | |
| | | LTISSLQPDDFATYYCLEY | | | | | | | |
| | | SSYIRTFGQGTKVEIK | | | | | | | |
| mAb11- | VH | EVQLVESGGGLVQPGRS | 155 | DYAMH | 156 | GISWKGDIGGYV | 157 | SYGSGSFYNAFDS | 158 |
| 0931 | | LRLSCAASGFTFHDYAMH | | | | DSVKG | | | |
| | | WVRQVPGKGLEWVSGIS | | | | | | | |
| | | WKGDIGGYVDSVKGRFTI | | | | | | | |
| | | SRDNEKNSLYLQMNSLR | | | | | | | |
| | | AEDTALYYCVKSYGSGSF | | | | | | | |
| | | YNAFDSWGQGTLVTVSS | | | | | | | |
| mAb11- | VL | DIQMTQSPSTLSASVGDR | 159 | RASQSISSW | 160 | KASRLDR | 161 | LEYSSYIRT | 162 |
| 0931 | | VTITCRASQSISSWLAWY | | LA | | | | | |
| | | QQKPGKAPKFLIYKASRL | | | | | | | |
| | | DRGLPSRFSGSGSGTEF | | | | | | | |
| | | SLTISSLQPDDFATYYCLE | | | | | | | |
| | | YSSYIRTFGQGTKVEIK | | | | | | | |
| mAb11- | VH | EVQLVESGGGLVQPGRS | 163 | DYAMH | 164 | GISWKGDIGGYV | 165 | SYGSGSFYNAFDS | 166 |
| 0932 | | LRLSCAASGFTFHDYAMH | | | | DSVKG | | | |
| | | WVRQVPGKGLEWVSGIS | | | | | | | |
| | | WKGDIGGYVDSVKGRFTI | | | | | | | |
| | | SRDNEKNSLYLQMNSLR | | | | | | | |
| | | AEDTALYYCVKSYGSGSF | | | | | | | |
| | | YNAFDSWGQGTLVTVSS | | | | | | | |
| mAb11- | VL | DIQMTQSPSTLSASVGDR | 167 | RASQSISSW | 168 | KASRLDR | 169 | LEYSSYIRT | 170 |
| 0932 | | VTITCRASQSISSWLAWY | | LA | | | | | |
| | | QQKPGKAPKFLIYKASRL | | | | | | | |
| | | DRGMPSRFSGSGSGTEF | | | | | | | |
| | | SLTISSLQPDDFATYYCLE | | | | | | | |
| | | YSSYIRTFGQGTKVEIK | | | | | | | |
| mAb11- | VH | EVQLVESGGGLVQPGRS | 171 | DYAMH | 172 | GISWKGDIGGYV | 173 | SYGSGSFYNAFDS | 174 |
| 0933 | | LRLSCAASGFTFHDYAMH | | | | DSVKG | | | |
| | | WVRQVPGKGLEWVSGIS | | | | | | | |
| | | WKGDIGGYVDSVKGRFTI | | | | | | | |
| | | SRDNEKNSLYLQMNSLR | | | | | | | |
| | | AEDTALYYCVKSYGSGSF | | | | | | | |
| | | YNAFDSWGQGTLVTVSS | | | | | | | |
| mAb11- | VL | DIQMTQSPSTLSASVGDR | 175 | RASQSISSW | 176 | KASRLDR | 177 | LEYSSYIRT | 178 |
| 0933 | | VTITCRASQSISSWLAWY | | LA | | | | | |
| | | QQKPGKAPKFLIYKASRL | | | | | | | |
| | | DRGNPSRFSGSGSGTEF | | | | | | | |
| | | SLTISSLQPDDFATYYCLE | | | | | | | |
| | | YSSYIRTFGQGTKVEIK | | | | | | | |
| mAb11- | VH | EVQLVESGGGLVQPGRS | 179 | DYAMH | 180 | GISWKGDIGGYV | 181 | SYGSGSFYNAFDS | 182 |
| 0935 | | LRLSCAASGFTFHDYAMH | | | | DSVKG | | | |
| | | WVRQVPGKGLEWVSGIS | | | | | | | |
| | | WKGDIGGYVDSVKGRFTI | | | | | | | |
| | | SRDNEKNSLYLQMNSLR | | | | | | | |
| | | AEDTALYYCVKSYGSGSF | | | | | | | |
| | | YNAFDSWGQGTLVTVSS | | | | | | | |
| mAb11- | VL | DIQMTQSPSTLSASVGDR | 183 | RASQSISSW | 184 | KASRLDR | 185 | LEYSSYIRT | 186 |
| 0935 | | VTITCRASQSISSWLAWY | | LA | | | | | |
| | | QQKPGKAPKFLIYKASRL | | | | | | | |
| | | DRGQPSRFSGSGSGTEF | | | | | | | |
| | | SLTISSLQPDDFATYYCLE | | | | | | | |
| | | YSSYIRTFGQGTKVEIK | | | | | | | |
| mAb11- | VH | EVQLVESGGGLVQPGRS | 187 | DYAMH | 188 | GISWKGDIGGYV | 189 | SYGSGSFYNAFDS | 190 |
| 0937 | | LRLSCAASGFTFHDYAMH | | | | DSVKG | | | |
| | | WVRQVPGKGLEWVSGIS | | | | | | | |
| | | WKGDIGGYVDSVKGRFTI | | | | | | | |
| | | SRDNEKNSLYLQMNSLR | | | | | | | |
| | | AEDTALYYCVKSYGSGSF | | | | | | | |
| | | YNAFDSWGQGTLVTVSS | | | | | | | |
| mAb11- | VL | DIQMTQSPSTLSASVGDR | 191 | RASQSISSW | 192 | KASRLDR | 193 | LEYSSYIRT | 194 |
| 0937 | | VTITCRASQSISSWLAWY | | LA | | | | | |
| | | QQKPGKAPKFLIYKASRL | | | | | | | |
| | | DRGSPSRFSGSGSGTEF | | | | | | | |
| | | SLTISSLQPDDFATYYCLE | | | | | | | |
| | | YSSYIRTFGQGTKVEIK | | | | | | | |
| mAb11- | VH | EVQLVESGGGLVQPGRS | 195 | DYAMH | 196 | GISWKGDIGGYV | 197 | SYGSGSFYNAFDS | 198 |
| 0938 | | LRLSCAASGFTFHDYAMH | | | | DSVKG | | | |
| | | WVRQVPGKGLEWVSGIS | | | | | | | |
| | | WKGDIGGYVDSVKGRFTI | | | | | | | |
| | | SRDNEKNSLYLQMNSLR | | | | | | | |
| | | AEDTALYYCVKSYGSGSF | | | | | | | |
| | | YNAFDSWGQGTLVTVSS | | | | | | | |
| mAb11- | VL | DIQMTQSPSTLSASVGDR | 199 | RASQSISSW | 200 | KASRLDR | 201 | LEYSSYIRT | 202 |
| 0938 | | VTITCRASQSISSWLAWY | | LA | | | | | |
| | | QQKPGKAPKFLIYKASRL | | | | | | | |
| | | DRGVPSRFSGSGSGTEF | | | | | | | |
| | | SLTISSLQPDDFATYYCLE | | | | | | | |
| | | YSSYIRTFGQGTKVEIK | | | | | | | |
| mAb11- | VH | EVQLVESGGGLVQPGRS | 203 | DYAMH | 204 | GISWKGDIGGYV | 205 | SYGSGSFYNAFDS | 206 |
| 0939 | | LRLSCAASGFTFHDYAMH | | | | DSVKG | | | |
| | | WVRQVPGKGLEWVSGIS | | | | | | | |
| | | WKGDIGGYVDSVKGRFTI | | | | | | | |
| | | SRDNEKNSLYLQMNSLR | | | | | | | |
| | | AEDTALYYCVKSYGSGSF | | | | | | | |
| | | YNAFDSWGQGTLVTVSS | | | | | | | |
| mAb11- | VL | DIQMTQSPSTLSASVGDR | 207 | RASQSISSW | 208 | KASRLDR | 209 | LEYSSYIRT | 210 |
| 0939 | | VTITCRASQSISSWLAWY | | LA | | | | | |
| | | QQKPGKAPKFLIYKASRL | | | | | | | |
| | | DRGWPSRFSGSGSGTEF | | | | | | | |
| | | SLTISSLQPDDFATYYCLE | | | | | | | |
| | | YSSYIRTFGQGTKVEIK | | | | | | | |
| mAb11- | VH | EVQLVESGGGLVQPGRS | 211 | DYAMH | 212 | GISWKGDIGGYA | 213 | SYGSGSFYNAFDS | 214 |
| 0962 | | LRLSCAASKFTFHDYAMH | | | | DSVKG | | | |
| | | WVRQAPGKGLEWVSGIS | | | | | | | |
| | | WKGDIGGYADSVKGRFTI | | | | | | | |
| | | SRDNAKNSLYLQMNSLR | | | | | | | |
| | | AEDTALYYCAKSYGSGSF | | | | | | | |
| | | YNAFDSWGQGTLVTVSS | | | | | | | |
| mAb11- | VL | DIQMTQSPSTLSASVGDR | 215 | RASQSISSW | 216 | KASKLDR | 217 | LEYSSYIRT | 218 |
| 0962 | | VTITCRASQSISSWLAWY | | | | LA | | | |
| | | QQKPGKAPKFLIYKASKL | | | | | | | |
| | | DRGTPSRFSGSGSGTEF | | | | | | | |
| | | TLTISSLQPDDFATYYCLE | | | | | | | |
| | | YSSYIRTFGQGTKVEIK | | | | | | | |
| mAb11- | VH | EVQLVESGGGLVQPGRS | 219 | DYAMH | 220 | GISWKGDIGGYA | 221 | SYGSGSFYNAFDS | 222 |
| 0965 | | LRLSCAASKFTFHDYAMH | | | | DSVKG | | | |
| | | WVRQAPGKGLEWVSGIS | | | | | | | |
| | | WKGDIGGYADSVKGRFTI | | | | | | | |
| | | SRDNAKNSLYLQMNSLR | | | | | | | |
| | | AEDTALYYCAKSYGSGSF | | | | | | | |
| | | YNAFDSWGQGTLVTVSS | | | | | | | |
| mAb11- | VL | DIQMTQSPSTLSASVGDR | 223 | RASQSISSW | 224 | KASKLER | 225 | LEYSSYIRT | 226 |
| 0965 | | VTITCRASQSISSWLAWY | | LA | | | | | |
| | | QQKPGKAPKFLIYKASKL | | | | | | | |
| | | ERGTPSRFSGSGSGTEF | | | | | | | |
| | | TLTISSLQPDDFATYYCLE | | | | | | | |
| | | YSSYIRTFGQGTKVEIK | | | | | | | |
| mAb11- | VH | EVQLVESGGGLVQPGRS | 227 | DYAMH | 228 | GISWKGDIGGYV | 229 | SYGSGSFYNAFDS | 230 |
| 1021 | | LRLSCAASKFTFHDYAMH | | | | DSVKG | | | |
| | | WVRQAPGKGLEWVSGIS | | | | | | | |
| | | WKGDIGGYVDSVKGRFTI | | | | | | | |
| | | SRDNAKNSLYLQMNSLR | | | | | | | |
| | | AEDTALYYCAKSYGSGSF | | | | | | | |
| | | YNAFDSWGQGTLVTVSS | | | | | | | |
| mAb11- | VL | DIQMTQSPSTLSASVGDR | 231 | RASQSISSW | 232 | KASKLDR | 233 | LEYSSYIRT | 234 |
| 1021 | | VTITCRASQSISSWLAWY | | LA | | | | | |
| | | QQKPGKAPKFLIYKASKL | | | | | | | |
| | | DRGTPSRFSGSGSGTEF | | | | | | | |
| | | TLTISSLQPDDFATYYCLE | | | | | | | |
| | | YSSYIRTFGQGTKVEIK | | | | | | | |
| mAb11- | VH | EVQLVESGGGLVQPGRS | 235 | DYAMH | 236 | GISWKGDIGGYV | 237 | SYGSGSFYNAFDS | 238 |
| 1024 | | LRLSCAASKFTFHDYAMH | | | | DSVKG | | | |
| | | WVRQAPGKGLEWVSGIS | | | | | | | |
| | | WKGDIGGYVDSVKGRFTI | | | | | | | |
| | | SRDNAKNSLYLQMNSLR | | | | | | | |
| | | AEDTALYYCAKSYGSGSF | | | | | | | |
| | | YNAFDSWGQGTLVTVSS | | | | | | | |
| mAb11- | VL | DIQMTQSPSTLSASVGDR | 239 | RASQSISSW | 240 | KASKLER | 241 | LEYSSYIRT | 242 |
| 1024 | | VTITCRASQSISSWLAWY | | LA | | | | | |
| | | QQKPGKAPKFLIYKASKL | | | | | | | |
| | | ERGTPSRFSGSGSGTEF | | | | | | | |
| | | TLTISSLQPDDFATYYCLE | | | | | | | |
| | | YSSYIRTFGQGTKVEIK | | | | | | | |
| mAb11- | VH | EVQLVESGGGLVQPGRS | 243 | DYAMH | 244 | GISWKGDIGGYV | 245 | SYGSGSFYNAFDS | 246 |
| 1030 | | LRLSCAASKFTFHDYAMH | | | | DSVKG | | | |
| | | WVRQVPGKGLEWVSGIS | | | | | | | |
| | | WKGDIGGYVDSVKGRFTI | | | | | | | |
| | | SRDNAKNSLYLQMNSLR | | | | | | | |
| | | AEDTALYYCAKSYGSGSF | | | | | | | |
| | | YNAFDSWGQGTLVTVSS | | | | | | | |
| mAb11- | VL | DIQMTQSPSTLSASVGDR | 247 | RASQSISSW | 248 | KASKLDR | 249 | LEYSSYIRT | 250 |
| 1030 | | VTITCRASQSISSWLAWY | | LA | | | | | |
| | | QQKPGKAPKFLIYKASKL | | | | | | | |
| | | DRGTPSRFSGSGSGTEF | | | | | | | |
| | | TLTISSLQPDDFATYYCLE | | | | | | | |
| | | YSSYIRTFGQGTKVEIK | | | | | | | |
| mAb11- | VH | EVQLVESGGGLVQPGRS | 251 | DYAMH | 252 | GISWKGDIGGYV | 253 | SYGSGSFYNAFDS | 254 |
| 1033 | | LRLSCAASKFTFHDYAMH | | | | DSVKG | | | |
| | | WVRQVPGKGLEWVSGIS | | | | | | | |
| | | WKGDIGGYVDSVKGRFTI | | | | | | | |
| | | SRDNAKNSLYLQMNSLR | | | | | | | |
| | | AEDTALYYCAKSYGSGSF | | | | | | | |
| | | YNAFDSWGQGTLVTVSS | | | | | | | |
| mAb11- | VL | DIQMTQSPSTLSASVGDR | 255 | RASQSISSW | 256 | KASKLER | 257 | LEYSSYIRT | 258 |
| 1033 | | VTITCRASQSISSWLAWY | | LA | | | | | |
| | | QQKPGKAPKFLIYKASKL | | | | | | | |
| | | ERGTPSRFSGSGSGTEF | | | | | | | |
| | | TLTISSLQPDDFATYYCLE | | | | | | | |
| | | YSSYIRTFGQGTKVEIK | | | | | | | |
| mAb11- | VH | EVQLVESGGGLVQPGRS | 259 | DYAMH | 260 | GISWKGDIGGYV | 261 | SYGSGSFYNAFDS | 262 |
| 1039 | | LKLSCAASKFTFHDYAMH | | | | DSVKG | | | |
| | | WVRQAPGKGLEWVSGIS | | | | | | | |
| | | WKGDIGGYVDSVKGRFTI | | | | | | | |
| | | SRDNAKNSLYLQMNSLR | | | | | | | |
| | | AEDTALYYCAKSYGSGSF | | | | | | | |
| | | YNAFDSWGQGTLVTVSS | | | | | | | |
| mAb11- | VL | DIQMTQSPSTLSASVGDR | 263 | RASQSISSW | 264 | KASKLDR | 265 | LEYSSYIRT | 266 |
| 1039 | | VTITCRASQSISSWLAWY | | LA | | | | | |
| | | QQKPGKAPKFLIYKASKL | | | | | | | |
| | | DRGTPSRFSGSGSGTEF | | | | | | | |
| | | TLTISSLQPDDFATYYCLE | | | | | | | |
| | | YSSYIRTFGQGTKVEIK | | | | | | | |
| mAb11- | VH | EVQLVESGGGLVQPGRS | 267 | DYAMH | 268 | GISWKGDIGGYV | 269 | SYGSGSFYNAFDS | 270 |
| 1042 | | LKLSCAASKFTFHDYAMH | | | | DSVKG | | | |
| | | WVRQAPGKGLEWVSGIS | | | | | | | |
| | | WKGDIGGYVDSVKGRFTI | | | | | | | |
| | | SRDNAKNSLYLQMNSLR | | | | | | | |
| | | AEDTALYYCAKSYGSGSF | | | | | | | |
| | | YNAFDSWGQGTLVTVSS | | | | | | | |
| mAb11- | VL | DIQMTQSPSTLSASVGDR | 271 | RASQSISSW | 272 | KASKLER | 273 | LEYSSYIRT | 274 |
| 1042 | | VTITCRASQSISSWLAWY | | LA | | | | | |
| | | QQKPGKAPKFLIYKASKL | | | | | | | |
| | | ERGTPSRFSGSGSGTEF | | | | | | | |
| | | TLTISSLQPDDFATYYCLE | | | | | | | |
| | | YSSYIRTFGQGTKVEIK | | | | | | | |
| mAb11- | VH | EVQLVESGGGLVQPGRS | 275 | DYAMH | 276 | GISWKGDIGGYV | 277 | SYGSGSFYNAFDS | 278 |
| 1073 | | LRLSCAASGFTFHDYAMH | | | | DSVKG | | | |
| | | WVRQVPGKGLEWVSGIS | | | | | | | |
| | | WKGDIGGYVDSVKGRFTI | | | | | | | |
| | | SRDNAKNSLYLQMNSLR | | | | | | | |
| | | AEDTALYYCAKSYGSGSF | | | | | | | |
| | | YNAFDSWGQGTLVTVSS | | | | | | | |
| mAb11- | VL | DIQMTQSPSTLSASVGDR | 279 | RASQSISSW | 280 | KASKLDR | 281 | LEYSSYIRT | 282 |
| 1073 | | VTITCRASQSISSWLAWY | | LA | | | | | |
| | | QQKPGKAPKFLIYKASKL | | | | | | | |
| | | DRGTPSRFSGSGSGTEF | | | | | | | |
| | | TLTISSLQPDDFATYYCLE | | | | | | | |
| | | YSSYIRTFGQGTKVEIK | | | | | | | |
| mAb11- | VH | EVQLVESGGGLVQPGRS | 283 | DYAMH | 284 | GISWKGDIGGYV | 285 | SYGSGSFYNAFDS | 286 |
| 1076 | | LRLSCAASGFTFHDYAMH | | | | DSVKG | | | |
| | | WVRQVPGKGLEWVSGIS | | | | | | | |
| | | WKGDIGGYVDSVKGRFTI | | | | | | | |
| | | SRDNAKNSLYLQMNSLR | | | | | | | |
| | | AEDTALYYCAKSYGSGSF | | | | | | | |
| | | YNAFDSWGQGTLVTVSS | | | | | | | |
| mAb11- | VL | DIQMTQSPSTLSASVGDR | 287 | RASQSISSW | 288 | KASKLER | 289 | LEYSSYIRT | 290 |
| 1076 | | VTITCRASQSISSWLAWY | | LA | | | | | |
| | | QQKPGKAPKFLIYKASKL | | | | | | | |
| | | ERGTPSRFSGSGSGTEF | | | | | | | |
| | | TLTISSLQPDDFATYYCLE | | | | | | | |
| | | YSSYIRTFGQGTKVEIK | | | | | | | |
| mAb11- | VH | EVQLVESGGGLVQPGRS | 291 | DYAMH | 292 | GISWKGDIGGYV | 293 | SYGSGSFYNAFDS | 294 |
| 1091 | | LKLSCAASGFTFHDYAMH | | | | DSVKG | | | |
| | | WVRQVPGKGLEWVSGIS | | | | | | | |
| | | WKGDIGGYVDSVKGRFTI | | | | | | | |
| | | SRDNAKNSLYLQMNSLR | | | | | | | |
| | | AEDTALYYCAKSYGSGSF | | | | | | | |
| | | YNAFDSWGQGTLVTVSS | | | | | | | |
| mAb11- | VL | DIQMTQSPSTLSASVGDR | 295 | RASQSISSW | 296 | KASKLDR | 297 | LEYSSYIRT | 298 |
| 1091 | | VTITCRASQSISSWLAWY | | LA | | | | | |
| | | QQKPGKAPKFLIYKASKL | | | | | | | |
| | | DRGTPSRFSGSGSGTEF | | | | | | | |
| | | TLTISSLQPDDFATYYCLE | | | | | | | |
| | | YSSYIRTFGQGTKVEIK | | | | | | | |
| mAb11- | VH | EVQLVESGGGLVQPGRS | 299 | DYAMH | 300 | GISWKGDIGGYV | 301 | SYGSGSFYNAFDS | 302 |
| 1094 | | LKLSCAASGFTFHDYAMH | | | | DSVKG | | | |
| | | WVRQVPGKGLEWVSGIS | | | | | | | |
| | | WKGDIGGYVDSVKGRFTI | | | | | | | |
| | | SRDNAKNSLYLQMNSLR | | | | | | | |
| | | AEDTALYYCAKSYGSGSF | | | | | | | |
| | | YNAFDSWGQGTLVTVSS | | | | | | | |
| mAb11- | VL | DIQMTQSPSTLSASVGDR | 303 | RASQSISSW | 304 | KASKLER | 305 | LEYSSYIRT | 306 |
| 1094 | | VTITCRASQSISSWLAWY | | LA | | | | | |
| | | QQKPGKAPKFLIYKASKL | | | | | | | |
| | | ERGTPSRFSGSGSGTEF | | | | | | | |
| | | TLTISSLQPDDFATYYCLE | | | | | | | |
| | | YSSYIRTFGQGTKVEIK | | | | | | | |
| mAb11- | VH | EVQLVESGGGLVQPGRS | 307 | DYAMH | 308 | GISWKGDIGGYV | 309 | SYGSGSFYNAFDS | 310 |
| 1233 | | LRLSCAASGFKFHDYAMH | | | | DSVKG | | | |
| | | WVRQVPGKGLEWVSGIS | | | | | | | |
| | | WKGDIGGYVDSVKGRFTI | | | | | | | |
| | | SRDNEKNSLYLQMNSLR | | | | | | | |
| | | AEDTALYYCVKSYGSGSF | | | | | | | |
| | | YNAFDSWGQGTLVTVSS | | | | | | | |
| mAb11- | VL | DIQMTQSPSTLSASVGDR | 311 | RASQSISSW | 312 | KASRLDR | 313 | LEYSSYIRT | 314 |
| 1233 | | VTITCRASQSISSWLAWY | | LA | | | | | |
| | | QQKPGKAPKFLIYKASRL | | | | | | | |
| | | DRGTPSRFSGSGSGTEF | | | | | | | |
| | | SLTISSLQPDDFATYYCLE | | | | | | | |
| | | YSSYIRTFGQGTKVEIK | | | | | | | |
| mAb11- | VH | EVQLVESGGGLVQPGRS | 315 | DYAMH | 316 | GISWKGDIGGYV | 317 | SYGSGSFYNAFDS | 318 |
| 1254 | | LRLSCAASGFTFHDYAMH | | | | DSVKG | | | |
| | | WVRQVPGKGLEWVSGIS | | | | | | | |
| | | WKGDIGGYVDSVKGRFTI | | | | | | | |
| | | SRDNDKNSLYLQMNSLR | | | | | | | |
| | | AEDTALYYCVKSYGSGSF | | | | | | | |
| | | YNAFDSWGQGTLVTVSS | | | | | | | |
| mAb11- | VL | DIQMTQSPSTLSASVGDR | 319 | RASQSISSW | 320 | KASRLDR | 321 | LEYSSYIRT | 322 |
| 1254 | | VTITCRASQSISSWLAWY | | LA | | | | | |
| | | QQKPGKAPKFLIYKASRL | | | | | | | |
| | | DRGTPSRFSGSGSGTEF | | | | | | | |
| | | SLTISSLQPDDFATYYCLE | | | | | | | |
| | | YSSYIRTFGQGTKVEIK | | | | | | | |
| mAb11- | VH | EVQLVESGGGLVQPGRS | 323 | DYAMH | 324 | GISWKGDIGGYV | 325 | SYGSGSFYNAFDS | 326 |
| 1259 | | LRLSCAASGFTFHDYAMH | | | | DSVKG | | | |
| | | WVRQVPGKGLEWVSGIS | | | | | | | |
| | | WKGDIGGYVDSVKGRFTI | | | | | | | |
| | | SRDNEKNSLYLQMNSLR | | | | | | | |
| | | AEDTALYYCVKSYGSGSF | | | | | | | |
| | | YNAFDSWGQGTLVTVSS | | | | | | | |
| mAb11- | VL | DIQMTQSPSTLSASVGDR | 327 | RASQKISSW | 328 | KASRLDR | 329 | LEYSSYIRT | 330 |
| 1259 | | VTITCRASQKISSWLAWY | | LA | | | | | |
| | | QQKPGKAPKFLIYKASRL | | | | | | | |
| | | DRGTPSRFSGSGSGTEF | | | | | | | |
| | | SLTISSLQPDDFATYYCLE | | | | | | | |
| | | YSSYIRTFGQGTKVEIK | | | | | | | |
| mAb11- | VH | EVQLVESGGGLVQPGRS | 331 | DYAMH | 332 | GISWKGDIGGYV | 333 | SYGSGSFYNAFDS | 334 |
| 1260 | | LRLSCAASGFTFHDYAMH | | | | DSVKG | | | |
| | | WVRQVPGKGLEWVSGIS | | | | | | | |
| | | WKGDIGGYVDSVKGRFTI | | | | | | | |
| | | SRDNEKNSLYLQMNSLR | | | | | | | |
| | | AEDTALYYCVKSYGSGSF | | | | | | | |
| | | YNAFDSWGQGTLVTVSS | | | | | | | |
| mAb11- | VL | DIQMTQSPSTLSASVGDR | 335 | RASQQISS | 336 | KASRLDR | 337 | LEYSSYIRT | 338 |
| 1260 | | VTITCRASQQISSWLAWY | | WLA | | | | | |
| | | QQKPGKAPKFLIYKASRL | | | | | | | |
| | | DRGTPSRFSGSGSGTEF | | | | | | | |
| | | SLTISSLQPDDFATYYCLE | | | | | | | |
| | | YSSYIRTFGQGTKVEIK | | | | | | | |
| mAb11- | VH | EVQLVESGGGLVQPGRS | 339 | DYAMH | 340 | GISWKGDIGGYV | 341 | SYGSGSFYNAFDS | 342 |
| 1262 | | LRLSCAASGFTFHDYAMH | | | | DSVKG | | | |
| | | WVRQVPGKGLEWVSGIS | | | | | | | |
| | | WKGDIGGYVDSVKGRFTI | | | | | | | |
| | | SRDNEKNSLYLQMNSLR | | | | | | | |
| | | AEDTALYYCVKSYGSGSF | | | | | | | |
| | | YNAFDSWGQGTLVTVSS | | | | | | | |
| mAb11- | VL | DIQMTQSPSTLSASVGDR | 343 | RASQSIQS | 344 | KASRLDR | 345 | LEYSSYIRT | 346 |
| 1262 | | VTITCRASQSIQSWLAWY | | WLA | | | | | |
| | | QQKPGKAPKFLIYKASRL | | | | | | | |
| | | DRGTPSRFSGSGSGTEF | | | | | | | |
| | | SLTISSLQPDDFATYYCLE | | | | | | | |
| | | SSYIRTFGQGTKVEIK | | | | | | | |
| mAb11- | VH | EVQLVESGGGLVQPGRS | 347 | DYAMH | 348 | GISWKGDIGGYV | 349 | SYGSGSFYNAFDS | 350 |
| 1266 | | LRLSCAASGFTFHDYAMH | | | | DSVKG | | | |
| | | WVRQVPGKGLEWVSGIS | | | | | | | |
| | | WKGDIGGYVDSVKGRFTI | | | | | | | |
| | | SRDNEKNSLYLQMNSLR | | | | | | | |
| | | AEDTALYYCVKSYGSGSF | | | | | | | |
| | | YNAFDSWGQGTLVTVSS | | | | | | | |
| mAb11- | VL | DIQMTQSPSTLSASVGDR | 351 | RASQSISSW | 352 | KASRLDK | 353 | LEYSSYIRT | 354 |
| 1266 | | VTITCRASQSISSWLAWY | | LA | | | | | |
| | | QQKPGKAPKFLIYKASRL | | | | | | | |
| | | DKGTPSRFSGSGSGTEF | | | | | | | |
| | | SLTISSLQPDDFATYYCLE | | | | | | | |
| | | YSSYIRTFGQGTKVEIK | | | | | | | |
| mAb11- | VH | EVQLVESGGGLVQPGRS | 355 | DYAMH | 356 | GISWKGDIGGYV | 357 | SYGSGSFYNAFDS | 358 |
| 1268 | | LRLSCAASGFTFHDYAMH | | | | DSVKG | | | |
| | | WVRQVPGKGLEWVSGIS | | | | | | | |
| | | WKGDIGGYVDSVKGRFTI | | | | | | | |
| | | SRDNEKNSLYLQMNSLR | | | | | | | |
| | | AEDTALYYCVKSYGSGSF | | | | | | | |
| | | YNAFDSWGQGTLVTVSS | | | | | | | |
| mAb11- | VL | DIQMTQSPSTLSASVGDR | 359 | RASQSISSW | 360 | KASRLDR | 361 | LEYSSYIRT | 362 |
| 1268 | | VTITCRASQSISSWLAWY | | LA | | | | | |
| | | QQKPGKAPKFLIYKASRL | | | | | | | |
| | | DRGTPKRFSGSGSGTEF | | | | | | | |
| | | SLTISSLQPDDFATYYCLE | | | | | | | |
| | | YSSYIRTFGQGTKVEIK | | | | | | | |
| mAb11- | VH | EVQLVESGGGLVQPGRS | 363 | DYAMH | 364 | GISWKGDIGGYV | 365 | SYGSGSFYNAFDS | 366 |
| 1273 | | LRLSCAASGFTFHDYAMH | | | | DSVKG | | | |
| | | WVRQVPGKGLEWVSGIS | | | | | | | |
| | | WKGDIGGYVDSVKGRFTI | | | | | | | |
| | | SRDNEKNSLYLQMNSLR | | | | | | | |
| | | AEDTALYYCVKSYGSGSF | | | | | | | |
| | | YNAFDSWGQGTLVTVSS | | | | | | | |
| mAb11- | VL | DIQMTQSPSTLSASVGDR | 367 | RASQSISSW | 368 | KASRLDR | 369 | LEYSSYIRT | 370 |
| 1273 | | VTITCRASQSISSWLAWY | | LA | | | | | |
| | | QQKPGKAPKFLIYKASRL | | | | | | | |
| | | DRGTPSRFSGSGSGTEF | | | | | | | |
| | | SLTISELQPDDFATYYCLE | | | | | | | |
| | | YSSYIRTFGQGTKVEIK | | | | | | | |
| mAb11- | VH | EVQLVESGGGLVQPGRS | 371 | DYAMH | 372 | GISWKGDIGGYV | 373 | SYGSGSFYNAFDS | 374 |
| 1275 | | LRLSCAASGFTFHDYAMH | | | | DSVKG | | | |
| | | WVRQVPGKGLEWVSGIS | | | | | | | |
| | | WKGDIGGYVDSVKGRFTI | | | | | | | |
| | | SRDNEKNSLYLQMNSLR | | | | | | | |
| | | AEDTALYYCVKSYGSGSF | | | | | | | |
| | | YNAFDSWGQGTLVTVSS | | | | | | | |
| mAb11- | VL | DIQMTQSPSTLSASVGDR | 375 | RASQSISSW | 376 | KASRLDR | 377 | LEYSSYIRT | 378 |
| 1275 | | VTITCRASQSISSWLAWY | | LA | | | | | |
| | | QQKPGKAPKFLIYKASRL | | | | | | | |
| | | DRGTPSRFSGSGSGTEF | | | | | | | |
| | | SLTISSLTPDDFATYYCLE | | | | | | | |
| | | YSSYIRTFGQGTKVEIK | | | | | | | |
| mAb11- | VH | EVQLVESGGGLVQPGRS | 379 | DYAMH | 380 | GISWKGDIGGYV | 381 | SYGSGSFYNAFDS | 382 |
| 1276 | | LRLSCAASGFTFHDYAMH | | | | DSVKG | | | |
| | | WVRQVPGKGLEWVSGIS | | | | | | | |
| | | WKGDIGGYVDSVKGRFTI | | | | | | | |
| | | SRDNEKNSLYLQMNSLR | | | | | | | |
| | | AEDTALYYCVKSYGSGSF | | | | | | | |
| | | YNAFDSWGQGTLVTVSS | | | | | | | |
| mAb11- | VL | DIQMTQSPSTLSASVGDR | 383 | RASQSISSW | 384 | KASRLDR | 385 | LEYSSYIRT | 386 |
| 1276 | | VTITCRASQSISSWLAWY | | LA | | | | | |
| | | QQKPGKAPKFLIYKASRL | | | | | | | |
| | | DRGTPSRFSGSGSGTEF | | | | | | | |
| | | SLTISSLSPDDFATYYCLE | | | | | | | |
| | | YSSYIRTFGQGTKVEIK | | | | | | | |
| mAb11- | VH | EVQLVESGGGLVQPGRS | 387 | DYAMH | 388 | GISWKGDIGGYV | 389 | SYGSGSFYNAFDS | 390 |
| 1278 | | LRLSCAASGFTFHDYAMH | | | | DSVKG | | | |
| | | WVRQVPGKGLEWVSGIS | | | | | | | |
| | | WKGDIGGYVDSVKGRFTI | | | | | | | |
| | | SRDNEKNSLYLQMNSLR | | | | | | | |
| | | AEDTALYYCVKSYGSGSF | | | | | | | |
| | | YNAFDSWGQGTLVTVSS | | | | | | | |
| mAb11- | VL | DIQMTQSPSTLSASVGDR | 391 | RASQSISSW | 392 | KASRLDR | 393 | LEYQSYIRT | 394 |
| 1278 | | VTITCRASQSISSWLAWY | | LA | | | | | |
| | | QQKPGKAPKFLIYKASRL | | | | | | | |
| | | DRGTPSRFSGSGSGTEF | | | | | | | |
| | | SLTISSLQPDDFATYYCLE | | | | | | | |
| | | YQSYIRTFGQGTKVEIK | | | | | | | |
| mAb11- | VH | EVQLVESGGGLVQPGRS | 395 | DYAMH | 396 | GISWKGDIGGYV | 397 | SYGSGSFYNAFDS | 398 |
| 1279 | | LRLSCAASGFTFHDYAMH | | | | DSVKG | | | |
| | | WVRQVPGKGLEWVSGIS | | | | | | | |
| | | WKGDIGGYVDSVKGRFTI | | | | | | | |
| | | SRDNEKNSLYLQMNSLR | | | | | | | |
| | | AEDTALYYCVKSYGSGSF | | | | | | | |
| | | YNAFDSWGQGTLVTVSS | | | | | | | |
| mAb11- | VL | DIQMTQSPSTLSASVGDR | 399 | RASQSISSW | 400 | KASRLDR | 401 | LEYKSYIRT | 402 |
| 1279 | | VTITCRASQSISSWLAWY | | LA | | | | | |
| | | QQKPGKAPKFLIYKASRL | | | | | | | |
| | | DRGTPSRFSGSGSGTEF | | | | | | | |
| | | SLTISSLQPDDFATYYCLE | | | | | | | |
| | | YKSYIRTFGQGTKVEIK | | | | | | | |
| mAb11- | VH | EVQLVESGGGLVQPGRS | 403 | DYAMH | 404 | GISWKGDIGGYV | 405 | SYGSGSFYNAFDS | 406 |
| 1282 | | LRLSCAASGFTFHDYAMH | | | | DSVKG | | | |
| | | WVRQVPGKGLEWVSGIS | | | | | | | |
| | | WKGDIGGYVDSVKGRFTI | | | | | | | |
| | | SRDNEKNSLYLQMNSLR | | | | | | | |
| | | AEDTALYYCVKSYGSGSF | | | | | | | |
| | | YNAFDSWGQGTLVTVSS | | | | | | | |
| mAb11- | VL | DIQMTQSPSTLSASVGDR | 407 | RASQSISSW | 408 | KASRLDR | 409 | LEYSSWIRT | 410 |
| 1282 | | VTITCRASQSISSWLAWY | | LA | | | | | |
| | | QQKPGKAPKFLIYKASRL | | | | | | | |
| | | DRGTPSRFSGSGSGTEF | | | | | | | |
| | | SLTISSLQPDDFATYYCLE | | | | | | | |
| | | YSSWIRTFGQGTKVEIK | | | | | | | |
| mAb11- | VH | EVQLVESGGGLVQPGRS | 411 | DYAMH | 412 | GISWKGDIGGYV | 413 | SYGSGSFYNAFDS | 414 |
| 1286 | | LRLSCAASGFTFHDYAMH | | | | DSVKG | | | |
| | | WVRQVPGKGLEWVSGIS | | | | | | | |
| | | WKGDIGGYVDSVKGRFTI | | | | | | | |
| | | SRDNEKNSLYLQMNSLR | | | | | | | |
| | | AEDTALYYCVKSYGSGSF | | | | | | | |
| | | YNAFDSWGQGTLVTVSS | | | | | | | |
| mAb11- | VL | DIQMTQSPSTLSASVGDR | 415 | RASQSISSW | 416 | KASRLDR | 417 | LEYRSYIRT | 418 |
| 1286 | | VTITCRASQSISSWLAWY | | LA | | | | | |
| | | QQKPGKAPKFLIYKASRL | | | | | | | |
| | | DRGTPSRFSGSGSGTEF | | | | | | | |
| | | SLTIYDLQPDDFATYYCLE | | | | | | | |
| | | YRSYIRTFGQGTKVEIK | | | | | | | |
| mAb11- | VH | EVQLVESGGGLVQPGRS | 419 | DYAMH | 420 | GISWKGDIGGYV | 421 | SYGSGSFYNAFDS | 422 |
| 1288 | | LRLSCAASGFTFHDYAMH | | | | DSVKG | | | |
| | | WVRQVPGKGLEWVSGIS | | | | | | | |
| | | WKGDIGGYVDSVKGRFTI | | | | | | | |
| | | SRDNEKNSLYLQMNSLR | | | | | | | |
| | | AEDTALYYCVKSYGSGSF | | | | | | | |
| | | YNAFDSWGQGTLVTVSS | | | | | | | |
| mAb11- | VL | DIQMTQSPSTLSASVGDR | 423 | RASQSISSW | 424 | KASRLDR | 425 | LEYNSYIRT | 426 |
| 1288 | | VTITCRASQSISSWLAWY | | LA | | | | | |
| | | QQKPGKAPKFLIYKASRL | | | | | | | |
| | | DRGTPQRFSGSGSGTEF | | | | | | | |
| | | SLTIYSLQPDDFATYYCLE | | | | | | | |
| | | YNSYIRTFGQGTKVEIK | | | | | | | |
| mAb11- | VH | EVQLVESGGGLVQPGRS | 427 | DYAMH | 428 | GISWKGDIGGYV | 429 | SYGSGSFYNAFDS | 430 |
| 1344 | | LKLSCAASGFTFDDYAMH | | | | DSVKG | | | |
| | | WVRQVPGKGLEWVSGIS | | | | | | | |
| | | WKGDIGGYVDSVKGRFTI | | | | | | | |
| | | SRDNAKNSLYLQLNSLRA | | | | | | | |
| | | EDTALYYCAKSYGSGSFY | | | | | | | |
| | | NAFDSWGQGTLVTVSS | | | | | | | |
| mAb11- | VL | DIQMTQSPSTLSASAGDR | 431 | RASQSISSW | 432 | KASKLDR | 433 | LEYSSYIRT | 434 |
| 1344 | | VTITCRASQSISSWLAWY | | LA | | | | | |
| | | QQKPGKAPKFLIYKASKL | | | | | | | |
| | | DRGTPSRFSGSGSGTEF | | | | | | | |
| | | SLTISSLQPDDFATYYCLE | | | | | | | |
| | | YSSYIRTFGQGTKVEIK | | | | | | | |
| mAb11- | VH | EVQLVESGGGLVQPGRS | 435 | DYAMH | 436 | GISWRGDIGGYV | 437 | SYGSGSFYNAFDS | 438 |
| 1358 | | LKLSCAASGFTFDDYAMH | | | | DSVKG | | | |
| | | WVRQVPGKGLEWVSGIS | | | | | | | |
| | | WRGDIGGYVDSVKGRFTI | | | | | | | |
| | | SRDNAKNSLYLQLNSLRA | | | | | | | |
| | | EDTALYYCVKSYGSGSFY | | | | | | | |
| | | NAFDSWGQGTLVTVSS | | | | | | | |
| mAb11- | VL | DIQMTQSPSTLSASAGDR | 439 | RASQSISSW | 440 | KASKLDR | 441 | LEYSSYIRT | 442 |
| 1358 | | VTITCRASQSISSWLAWY | | LA | | | | | |
| | | QQKPGKAPKFLIYKASKL | | | | | | | |
| | | DRGTPSRFSGSGSGTEF | | | | | | | |
| | | SLTISSLQPDDFATYYCLE | | | | | | | |
| | | YSSYIRTFGQGTKVEIK | | | | | | | |
| mAb11- | VH | EVQLVESGGGLVQPGRS | 443 | DYAMH | 444 | GISWRGDIGGYV | 445 | SYGSGSFYNAFDS | 446 |
| 1360 | | LKLSCAASGFTFDDYAMH | | | | DSVKG | | | |
| | | WVRQVPGKGLEWVSGIS | | | | | | | |
| | | WRGDIGGYVDSVKGRFTI | | | | | | | |
| | | SRDNAKNSLYLQLNSLRA | | | | | | | |
| | | EDTALYYCVKSYGSGSFY | | | | | | | |
| | | NAFDSWGQGTLVTVSS | | | | | | | |
| mAb11- | VL | DIQMTQSPSTLSASVGDR | 447 | RASQSISSW | 448 | KASRLER | 449 | LEYSSYIRT | 450 |
| 1360 | | VTITCRASQSISSWLAWY | | LA | | | | | |
| | | QQKPGKAPKLLIYKASRL | | | | | | | |
| | | ERGTPSRFSGSGSGTEF | | | | | | | |
| | | SLTISSLQPDDFATYYCLE | | | | | | | |
| | | YSSYIRTFGQGTKVEIK | | | | | | | |
| mAb11- | VH | EVQLVESGGGLVQPGRS | 451 | DYAMH | 452 | GISWKGDIGGYV | 453 | SYGSGSFYNAFDS | 454 |
| 1389 | | LKLSCAASGFTFHDYAMH | | | | DSVKG | | | |
| | | WVRQAPGKGLEWVSGIS | | | | | | | |
| | | WKGDIGGYVDSVKGRFTI | | | | | | | |
| | | SRDNAKNSLYLQMNSLR | | | | | | | |
| | | AEDTALYYCAKSYGSGSF | | | | | | | |
| | | YNAFDSWGQGTLVTVSS | | | | | | | |
| mAb11- | VL | DIQMTQSPSTLSASAGDR | 455 | RASQSISSW | 456 | KASRLDR | 457 | LEYSSYIRT | 458 |
| 1389 | | VTITCRASQSISSWLAWY | | LA | | | | | |
| | | QQKPGKAPKFLIYKASRL | | | | | | | |
| | | DRGTPSRFSGSGSGTEF | | | | | | | |
| | | SLTISSLQPDDFATYYCLE | | | | | | | |
| | | YSSYIRTFGQGTKVEIK | | | | | | | |
| mAb11- | VH | EVQLVESGGGLVQPGRS | 1202 | DYAMH | 1203 | GISWKGDIGGYV | 1204 | SYGSGSFYNAFDS | 1205 |
| 0173 | | LRLSCAASGFTFHDYAMH | | | | DSVKG | | | |
| | | WVRQAPGKGLEWVSGIS | | | | | | | |
| | | WKGDIGGYVDSVKGRFTI | | | | | | | |
| | | SRDNAKNSLYLQMNSLR | | | | | | | |
| | | AEDTALYYCAKSYGSGSF | | | | | | | |
| | | YNAFDSWGQGTLVTVSS | | | | | | | |
| mAb11- | VL | DIQMTQSPSTLSASVGDR | 1206 | RASQSISSW | 1207 | KASKLDR | 1208 | LEYSSYIRT | 1209 |
| 0173 | | VTITCRASQSISSWLAWY | | LA | | | | | |
| | | QQKPGKAPKFLIYKASKL | | | | | | | |
| | | DRGTPSRFSGSGSGTEF | | | | | | | |
| | | TLTISSLQPDDFATYYCLE | | | | | | | |
| | | YSSYIRTFGQGTKVEIK | | | | | | | |
| mAb11- | VH | EVQLVESGGGLVQPGRS | 1210 | DYAMH | 1211 | GISWRGDIGGYV | 1212 | SYGSGSFYNAFDS | 1213 |
| 1204 | | LRLSCAASGFTFHDYAMH | | | | KSVKG | | | |
| | | WVRQVPGKGLEWVSGIS | | | | | | | |
| | | WRGDIGGYVKSVKGRFTI | | | | | | | |
| | | SRDNAKNSLYLQMNSLR | | | | | | | |
| | | AEDTALYYCVKSYGSGSF | | | | | | | |
| | | YNAFDSWGQGTLVTVSS | | | | | | | |
| mAb11- | VL | DIQMTQSPSTLSASVGDE | 1214 | RASQSISSW | 1215 | KASKLER | 1216 | LEYSSYIRT | 1217 |
| 1204 | | VTITCRASQSISSWLAWY | | LA | | | | | |
| | | QQKPGKAPKFLIYKASKL | | | | | | | |
| | | ERGTPSRFSGSGSGTEF | | | | | | | |
| | | SLTISSLQPDDFATYYCLE | | | | | | | |
| | | YSSYIRTFGQGTKVEIK | | | | | | | |
| mAb11- | VH | EVQLVESGGGLVQPGRS | 1218 | DYAMH | 1219 | GISWRGDIGGYA | 1220 | SYGSGSFYNAFDS | 1221 |
| 1495 | | LRLSCAASGFTFHDYAMH | | | | KSVKG | | | |
| | | WVRQVPGKGLEWVSGIS | | | | | | | |
| | | WRGDIGGYAKSVKGRFTI | | | | | | | |
| | | SRDNAKNSLYLQMNSLR | | | | | | | |
| | | AEDTALYYCVKSYGSGSF | | | | | | | |
| | | YNAFDSWGQGTLVTVSS | | | | | | | |
| mAb11- | VL | DIQMTQSPSTLSASVGDR | 1222 | RASQSISSW | 1223 | KASKLDR | 1224 | LEYSSYIRT | 1225 |
| 1495 | | VTITCRASQSISSWLAWY | | LA | | | | | |
| | | QQKPGKAPKFLIYKASKL | | | | | | | |
| | | DRGTPSRFSGSGSGTEF | | | | | | | |
| | | TLTISSLQPDDFATYYCLE | | | | | | | |
| | | YSSYIRTFGQGTKVEIK | | | | | | | |
| mAb11- | VH | EVQLVESGGGLVQPGRS | 1226 | DYAMH | 1227 | GISWRGDIGGYV | 1228 | SYGSGSFYNAFDS | 1229 |
| 1501 | | LRLSCAASGFTFDDYAMH | | | | KSVKG | | | |
| | | WVRQVPGKGLEWVSGIS | | | | | | | |
| | | WRGDIGGYVKSVKGRFTI | | | | | | | |
| | | SRDNAKNSLYLQMNSLR | | | | | | | |
| | | AEDTALYYCVKSYGSGSF | | | | | | | |
| | | YNAFDSWGQGTLVTVSS | | | | | | | |
| mAb11- | VL | DIQMTQSPSTLSASVGDR | 1230 | RASQSISSW | 1231 | KASKLDR | 1232 | LEYSSYIRT | 1233 |
| 1501 | | VTITCRASQSISSWLAWY | | LA | | | | | |
| | | QQKPGKAPKFLIYKASKL | | | | | | | |
| | | DRGTPSRFSGSGSGTEF | | | | | | | |
| | | TLTISSLQPDDFATYYCLE | | | | | | | |
| | | YSSYIRTFGQGTKVEIK | | | | | | | |
| mAb11- | VH | EVQLVESGGGLVQPGRS | 1234 | DYAMH | 1235 | GISWRGDIGGYV | 1236 | SYGSGSFYNAFDS | 1237 |
| 1502 | | LRLSCAASGFTFDDYAMH | | | | KSVKG | | | |
| | | WVRQVPGKGLEWVSGIS | | | | | | | |
| | | WRGDIGGYVKSVKGRFTI | | | | | | | |
| | | SRDNAKNSLYLQMNSLR | | | | | | | |
| | | AEDTALYYCVKSYGSGSF | | | | | | | |
| | | YNAFDSWGQGTLVTVSS | | | | | | | |
| mAb11- | VL | DIQMTQSPSTLSASVGDR | 1238 | RASQSISSW | 1239 | KASKLDR | 1240 | LEYSSYIRT | 1241 |
| 1502 | | VTITCRASQSISSWLAWY | | LA | | | | | |
| | | QQKPGKAPKLLIYKASKL | | | | | | | |
| | | DRGTPSRFSGSGSGTEF | | | | | | | |
| | | TLTISSLQPDDFATYYCLE | | | | | | | |
| | | YSSYIRTFGQGTKVEIK | | | | | | | |
| mAb11- | VH | EVQLVESGGGLVQPGRS | 1242 | DYAMH | 1243 | GISWRGDIGGYV | 1244 | SYGSGSFYNAFDS | 1245 |
| 1499 | | LRLSCAASGFTFHDYAMH | | | | KSVKG | | | |
| | | WVRQVPGKGLEWVSGIS | | | | | | | |
| | | WRGDIGGYVKSVKGRFTI | | | | | | | |
| | | SRDNAKNSLYLQMNSLR | | | | | | | |
| | | AEDTALYYCVKSYGSGSF | | | | | | | |
| | | YNAFDSWGQGTLVTVSS | | | | | | | |
| mAb11- | VL | DIQMTQSPSTLSASVGDR | 1246 | RASQSISSW | 1247 | KASKLDR | 1248 | LEYSSYIRT | 1249 |
| 1499 | | VTITCRASQSISSWLAWY | | LA | | | | | |
| | | QQKPGKAPKFLIYKASKL | | | | | | | |
| | | DRGTPSRFSGSGSGTEF | | | | | | | |
| | | TLTISSLQPDDFATYYCLE | | | | | | | |
| | | YSSYIRTFGQGTKVEIK | | | | | | | |

TABLE 3
Overview of anti-FX antibody VH, VL and CDR sequences

| | VH/VL | | | CDR1 | | CDR2 | | CDR3 | |
|---|---|---|---|---|---|---|---|---|---|
| mAb ID | Domain | Sequence | # | Sequence | # | Sequence | # | Sequence | # |
| mAb01- | VH | EVQLVQSGAEVKKPGE | 459 | TSWIV | 460 | MIDPSDSFTSYSPSFQ | 461 | LHYYHSEEFDV | 462 |
| 6723 | | SLRISCKGSGYSFTTSW | | | | G | | | |
| | | IVWVRQMPGKGLEWM | | | | | | | |
| | | GMIDPSDSFTSYSPSFQ | | | | | | | |
| | | GHVTISADKSISTAYLQ | | | | | | | |
| | | WSSLKASDTAMYYCAR | | | | | | | |
| | | LHYYHSEEFDVWGQGT | | | | | | | |
| | | LVTVSS | | | | | | | |
| mAb01- | VL | EIVLTQSPGTLSLSPGE | 463 | RASQSVSSS | 464 | GASSRAR | 465 | QQFGSSRLFT | 466 |
| 6723 | | RATLSCRASQSVSSSYL | | YLA | | | | | |
| | | AWYQQKPGQAPRLLIY | | | | | | | |
| | | GASSRARGIPDRFSGS | | | | | | | |
| | | GSGTDFTLTISRLEPED | | | | | | | |
| | | FAVYYCQQFGSSRLFTF | | | | | | | |
| | | GQGTKLEIK | | | | | | | |
| mAb01- | VH | EVQLVQSGAEVKKPGE | 467 | TSWIV | 468 | MIDPSDSFTSYSPSFQ | 469 | LHYYNSEEFDV | 470 |
| 8174 | | SLRISCKGSGYSFSTSW | | | | G | | | |
| | | IVWVRQMPGKGLEWM | | | | | | | |
| | | GMIDPSDSFTSYSPSFQ | | | | | | | |
| | | GHVTISADKSISTAYLQ | | | | | | | |
| | | WSSLKASDTAMYYCAR | | | | | | | |
| | | LHYYNSEEFDVWGQGT | | | | | | | |
| | | LVTVSS | | | | | | | |
| mAb01- | VL | EIVLTQSPGTLSLSPGE | 471 | RASQSVSSS | 472 | GQSSRTR | 473 | QQFGDSQLFT | 474 |
| 8174 | | RATLSCRASQSVSSSYL | | YLA | | | | | |
| | | AWYQQKPGQAPRLLIY | | | | | | | |
| | | GQSSRTRGIPDRFSGS | | | | | | | |
| | | GSGTDFTLTISRLEPED | | | | | | | |
| | | FAVYYCQQFGDSQLFT | | | | | | | |
| | | FGQGTKLEIK | | | | | | | |
| mAb01- | VH | EVQLVQSGAEVKKPGE | 475 | TSWIV | 476 | MIDPSDSFTSYSPSFQ | 477 | LHYYNSEEFDV | 478 |
| 8913 | | SLRISCKGSGYSFSTSW | | | | G | | | |
| | | IVWVRQMPGKGLEWM | | | | | | | |
| | | GMIDPSDSFTSYSPSFQ | | | | | | | |
| | | GHVTISADKSISTAYLQ | | | | | | | |
| | | WSSLKASDTAMYYCAR | | | | | | | |
| | | LHYYNSEEFDVWGQGT | | | | | | | |
| | | LVTVSS | | | | | | | |
| mAb01- | VL | EIVLTQSPGTLSLSAGE | 479 | RASQSVSSS | 480 | GASSRAR | 481 | QQFGSSRLFT | 482 |
| 8913 | | RATLSCRASQSVSSSYL | | YLA | | | | | |
| | | AWYQQKPGQAPRLLIY | | | | | | | |
| | | GASSRARGIPDRFSGS | | | | | | | |
| | | GSGTDFTLTISRLEPED | | | | | | | |
| | | FAVYYCQQFGSSRLFTF | | | | | | | |
| | | GQGTKLEIK | | | | | | | |
| mAb01- | VH | EVQLVQSGAEVKKPGE | 483 | TSWIS | 484 | MIDPSDSYTSYSPSFQ | 485 | LHYYNSEEFDV | 486 |
| 9772 | | SLRISCKGSGYSFSTSW | | | | G | | | |
| | | ISWVRQMPGKGLEWM | | | | | | | |
| | | GMIDPSDSYTSYSPSFQ | | | | | | | |
| | | GHVTISADKSISTAYLQ | | | | | | | |
| | | WSSLKASDTAMYYCAR | | | | | | | |
| | | LHYYNSEEFDVWGQGT | | | | | | | |
| | | MVTVSS | | | | | | | |
| mAb01- | VL | EIVLTQSPGTLSLSPGE | 487 | RASQSVSSS | 488 | GQSSRTR | 489 | QQYGDSQLFT | 490 |
| 9772 | | RATLSCRASQSVSSSYL | | YLA | | | | | |
| | | AWYQQKPGQAPRLLIY | | | | | | | |
| | | GQSSRTRGIPDRFSGS | | | | | | | |
| | | GSGTDFTLTISRLEPED | | | | | | | |
| | | FAVYYCQQYGDSQLFT | | | | | | | |
| | | FGQGTKLEIK | | | | | | | |
| mAb01- | VH | EVQLVQSGAEVKKPGE | 491 | TSWIS | 492 | MIDPSDSFTSYSPSFQ | 493 | LHYYNSEEFDV | 494 |
| 9778 | | SLRISCKGSGYSFSTSW | | | | G | | | |
| | | ISWVRQMPGKGLEWM | | | | | | | |
| | | GMIDPSDSFTSYSPSFQ | | | | | | | |
| | | GHVTISADKSISTAYLQ | | | | | | | |
| | | WSSLKASDTAMYYCAR | | | | | | | |
| | | LHYYNSEEFDVWGQGT | | | | | | | |
| | | LVTVSS | | | | | | | |
| mAb01- | VL | EIVLTQSPGTLSLSPGE | 495 | RASQSVSSS | 496 | GQSSRTR | 497 | QQYGDSQLFT | 498 |
| 9778 | | RATLSCRASQSVSSSYL | | YLA | | | | | |
| | | AWYQQKPGQAPRLLIY | | | | | | | |
| | | GQSSRTRGIPDRFSGS | | | | | | | |
| | | GSGTDFTLTISRLEPED | | | | | | | |
| | | FAVYYCQQYGDSQLFT | | | | | | | |
| | | FGQGTKLEIK | | | | | | | |
| mAb11- | VH | EVQLVQSGAEVKKPGE | 499 | TSWIS | 500 | MIDPSDSYTSYSPSFQ | 501 | LHYYNSEEFDV | 502 |
| 1114 | | SLRISCKGSGYSFSTSW | | | | G | | | |
| | | ISWVRQMPGKGLEWM | | | | | | | |
| | | GMIDPSDSYTSYSPSFQ | | | | | | | |
| | | GHVTISADKSISTAYLQ | | | | | | | |
| | | WSSLKASDTAMYYCAR | | | | | | | |
| | | LHYYNSEEFDVWGQGT | | | | | | | |
| | | MVTVSS | | | | | | | |
| mAb11- | VL | EIVLTQSPGTLSLSPGE | 503 | RASQSVSSS | 504 | GQSSRTR | 505 | QQFGDSQLFT | 506 |
| 1114 | | RATLSCRASQSVSSSYL | | YLA | | | | | |
| | | AWYQQKPGQAPRLLIY | | | | | | | |
| | | GQSSRTRGIPDRFSGS | | | | | | | |
| | | GSGTDFTLTISRLEPED | | | | | | | |
| | | FAVYYCQQFGDSQLFT | | | | | | | |
| | | FGQGTKLEIK | | | | | | | |
| mAb11- | VH | EVQLVQSGAEVKKPGE | 507 | TSWIS | 508 | MIDPSDSFTSYSPSFQ | 509 | LHYYHSEEFDV | 510 |
| 1115 | | SLRISCKGSGYSFTTSW | | | | G | | | |
| | | ISWVRQMPGKGLEWM | | | | | | | |
| | | GMIDPSDSFTSYSPSFQ | | | | | | | |
| | | GHVTISADKSISTAYLQ | | | | | | | |
| | | WSSLKASDTAMYYCAR | | | | | | | |
| | | LHYYHSEEFDVWGQGT | | | | | | | |
| | | MVTVSS | | | | | | | |
| mAb11- | VL | EIVLTQSPGTLSLSPGE | 511 | RASQSVSSS | 512 | GQSSRTR | 513 | QQFGDSQLFT | 514 |
| 1115 | | RATLSCRASQSVSSSYL | | YLA | | | | | |
| | | AWYQQKPGQAPRLLIY | | | | | | | |
| | | GQSSRTRGIPDRFSGS | | | | | | | |
| | | GSGTDFTLTISRLEPED | | | | | | | |
| | | FAVYYCQQFGDSQLFT | | | | | | | |
| | | FGQGTKLEIK | | | | | | | |
| mAb11- | VH | EVQLVQSGAEVKKPGE | 515 | TSWIS | 516 | MIDPSDSYTSYSPSFQ | 517 | LHYYHSEEFDV | 518 |
| 1116 | | SLRISCKGSGYSFTTSW | | | | G | | | |
| | | ISWVRQMPGKGLEWM | | | | | | | |
| | | GMIDPSDSYTSYSPSFQ | | | | | | | |
| | | GHVTISADKSISTAYLQ | | | | | | | |
| | | WSSLKASDTAMYYCAR | | | | | | | |
| | | LHYYHSEEFDVWGQGT | | | | | | | |
| | | LVTVSS | | | | | | | |
| mAb11- | VL | EIVLTQSPGTLSLSPGE | 519 | RASQSVSSS | 520 | GQSSRTR | 521 | QQFGDSQLFT | 522 |
| 1116 | | RATLSCRASQSVSSSYL | | YLA | | | | | |
| | | AWYQQKPGQAPRLLIY | | | | | | | |
| | | GQSSRTRGIPDRFSGS | | | | | | | |
| | | GSGTDFTLTISRLEPED | | | | | | | |
| | | FAVYYCQQFGDSQLFT | | | | | | | |
| | | FGQGTKLEIK | | | | | | | |
| mAb11- | VH | EVQLVQSGAEVKKPGE | 523 | TSWIS | 524 | MIDPSDSYTSYSPSFQ | 525 | LHYYHSEEFDV | 526 |
| 1117 | | SLRISCKGSGYSFTTSW | | | | G | | | |
| | | ISWVRQMPGKGLEWM | | | | | | | |
| | | GMIDPSDSYTSYSPSFQ | | | | | | | |
| | | GHVTISADKSISTAYLQ | | | | | | | |
| | | WSSLKASDTAMYYCAR | | | | | | | |
| | | LHYYHSEEFDVWGQGT | | | | | | | |
| | | MVTVSS | | | | | | | |
| mAb11- | VL | EIVLTQSPGTLSLSPGE | 527 | RASQSVSSS | 528 | GQSSRTR | 529 | QQFGDSQLFT | 530 |
| 1117 | | RATLSCRASQSVSSSYL | | YLA | | | | | |
| | | AWYQQKPGQAPRLLIY | | | | | | | |
| | | GQSSRTRGIPDRFSGS | | | | | | | |
| | | GSGTDFTLTISRLEPED | | | | | | | |
| | | FAVYYCQQFGDSQLFT | | | | | | | |
| | | FGQGTKLEIK | | | | | | | |
| mAb11- | VH | EVQLVQSGAEVKKPGE | 531 | TSWIS | 532 | MIDPSDSFTSYSPSFQ | 533 | LHYYNSEEFDV | 534 |
| 1118 | | SLRISCKGSGYSFSTSW | | | | G | | | |
| | | ISWVRQMPGKGLEWM | | | | | | | |
| | | GMIDPSDSFTSYSPSFQ | | | | | | | |
| | | GHVTISADKSISTAYLQ | | | | | | | |
| | | WSSLKASDTAMYYCAR | | | | | | | |
| | | LHYYNSEEFDVWGQGT | | | | | | | |
| | | LVTVSS | | | | | | | |
| mAb11- | VL | EIVLTQSPGTLSLSPGE | 535 | RASQSVSSS | 536 | GQSSRTR | 537 | QQFGDSQLFT | 538 |
| 1118 | | RATLSCRASQSVSSSYL | | YLA | | | | | |
| | | AWYQQKPGQAPRLLIY | | | | | | | |
| | | GQSSRTRGIPDRFSGS | | | | | | | |
| | | GSGTDFTLTISRLEPED | | | | | | | |
| | | FAVYYCQQFGDSQLFT | | | | | | | |
| | | FGQGTKLEIK | | | | | | | |
| mAb11- | VH | EVQLVQSGAEVKKPGE | 539 | TSWIV | 540 | MIDPSDSYTSYSPSFQ | 541 | LHYYNSEEFDV | 542 |
| 1119 | | SLRISCKGSGYSFSTSW | | | | G | | | |
| | | IVWVRQMPGKGLEWM | | | | | | | |
| | | GMIDPSDSYTSYSPSFQ | | | | | | | |
| | | GHVTISADKSISTAYLQ | | | | | | | |
| | | WSSLKASDTAMYYCAR | | | | | | | |
| | | LHYYNSEEFDVWGQGT | | | | | | | |
| | | LVTVSS | | | | | | | |
| mAb11- | VL | EIVLTQSPGTLSLSPGE | 543 | RASQSVSSS | 544 | GQSSRTR | 545 | QQFGDSQLFT | 546 |
| 1119 | | RATLSCRASQSVSSSYL | | YLA | | | | | |
| | | AWYQQKPGQAPRLLIY | | | | | | | |
| | | GQSSRTRGIPDRFSGS | | | | | | | |
| | | GSGTDFTLTISRLEPED | | | | | | | |
| | | FAVYYCQQFGDSQLFT | | | | | | | |
| | | FGQGTKLEIK | | | | | | | |
| mAb11- | VH | EVQLVQSGAEVKKPGE | 547 | TSWIV | 548 | MIDPSDSYTSYSPSFQ | 549 | LHYYNSEEFDV | 550 |
| 1120 | | SLRISCKGSGYSFSTSW | | | | G | | | |
| | | IVWVRQMPGKGLEWM | | | | | | | |
| | | GMIDPSDSYTSYSPSFQ | | | | | | | |
| | | GHVTISADKSISTAYLQ | | | | | | | |
| | | WSSLKASDTAMYYCAR | | | | | | | |
| | | LHYYNSEEFDVWGQGT | | | | | | | |
| | | MVTVSS | | | | | | | |
| mAb11- | VL | EIVLTQSPGTLSLSPGE | 551 | RASQSVSSS | 552 | GQSSRTR | 553 | QQFGDSQLFT | 554 |
| 1120 | | RATLSCRASQSVSSSYL | | YLA | | | | | |
| | | AWYQQKPGQAPRLLIY | | | | | | | |
| | | GQSSRTRGIPDRFSGS | | | | | | | |
| | | GSGTDFTLTISRLEPED | | | | | | | |
| | | FAVYYCQQFGDSQLFT | | | | | | | |
| | | FGQGTKLEIK | | | | | | | |
| mAb11- | VH | EVQLVQSGAEVKKPGE | 555 | TSWIV | 556 | MIDPSDSFTSYSPSFQ | 557 | LHYYNSEEFDV | 558 |
| 1121 | | SLRISCKGSGYSFSTSW | | | | G | | | |
| | | IVWVRQMPGKGLEWM | | | | | | | |
| | | GMIDPSDSFTSYSPSFQ | | | | | | | |
| | | GHVTISADKSISTAYLQ | | | | | | | |
| | | WSSLKASDTAMYYCAR | | | | | | | |
| | | LHYYNSEEFDVWGQGT | | | | | | | |
| | | LVTVSS | | | | | | | |
| mAb11- | VL | EIVLTQSPGTLSLSPGE | 559 | RASQSVSSS | 560 | GQSSRTR | 561 | QQYGDSQLFT | 562 |
| 1121 | | RATLSCRASQSVSSSYL | | YLA | | | | | |
| | | AWYQQKPGQAPRLLIY | | | | | | | |
| | | GQSSRTRGIPDRFSGS | | | | | | | |
| | | GSGTDFTLTISRLEPED | | | | | | | |
| | | FAVYYCQQYGDSQLFT | | | | | | | |
| | | FGQGTKLEIK | | | | | | | |
| mAb11- | VH | EVQLVQSGAEVKKPGE | 563 | TSWIS | 564 | MIDPSDSFTSYSPSFQ | 565 | LHYYHSEEFDV | 566 |
| 1122 | | SLRISCKGSGYSFTTSW | | | | G | | | |
| | | ISWVRQMPGKGLEWM | | | | | | | |
| | | GMIDPSDSFTSYSPSFQ | | | | | | | |
| | | GHVTISADKSISTAYLQ | | | | | | | |
| | | WSSLKASDTAMYYCAR | | | | | | | |
| | | LHYYHSEEFDVWGQGT | | | | | | | |
| | | MVTVSS | | | | | | | |
| mAb11- | VL | EIVLTQSPGTLSLSPGE | 567 | RASQSVSSS | 568 | GQSSRTR | 569 | QQYGDSQLFT | 570 |
| 1122 | | RATLSCRASQSVSSSYL | | YLA | | | | | |
| | | AWYQQKPGQAPRLLIY | | | | | | | |
| | | GQSSRTRGIPDRFSGS | | | | | | | |
| | | GSGTDFTLTISRLEPED | | | | | | | |
| | | FAVYYCQQYGDSQLFT | | | | | | | |
| | | FGQGTKLEIK | | | | | | | |
| mAb11- | VH | EVQLVQSGAEVKKPGE | 571 | TSWIS | 572 | MIDPSDSYTSYSPSFQ | 573 | LHYYHSEEFDV | 574 |
| 1123 | | SLRISCKGSGYSFTTSW | | | | G | | | |
| | | ISWVRQMPGKGLEWM | | | | | | | |
| | | GMIDPSDSYTSYSPSFQ | | | | | | | |
| | | GHVTISADKSISTAYLQ | | | | | | | |
| | | WSSLKASDTAMYYCAR | | | | | | | |
| | | LHYYHSEEFDVWGQGT | | | | | | | |
| | | LVTVSS | | | | | | | |
| mAb11- | VL | EIVLTQSPGTLSLSPGE | 575 | RASQSVSSS | 576 | GQSSRTR | 577 | QQYGDSQLFT | 578 |
| 1123 | | RATLSCRASQSVSSSYL | | YLA | | | | | |
| | | AWYQQKPGQAPRLLIY | | | | | | | |
| | | GQSSRTRGIPDRFSGS | | | | | | | |
| | | GSGTDFTLTISRLEPED | | | | | | | |
| | | FAVYYCQQYGDSQLFT | | | | | | | |
| | | FGQGTKLEIK | | | | | | | |
| mAb11- | VH | EVQLVQSGAEVKKPGE | 579 | TSWIS | 580 | MIDPSDSYTSYSPSFQ | 581 | LHYYHSEEFDV | 582 |
| 1124 | | SLRISCKGSGYSFTTSW | | | | G | | | |
| | | ISWVRQMPGKGLEWM | | | | | | | |
| | | GMIDPSDSYTSYSPSFQ | | | | | | | |
| | | GHVTISADKSISTAYLQ | | | | | | | |
| | | WSSLKASDTAMYYCAR | | | | | | | |
| | | LHYYHSEEFDVWGQGT | | | | | | | |
| | | MVTVSS | | | | | | | |
| mAb11- | VL | EIVLTQSPGTLSLSPGE | 583 | RASQSVSSS | 584 | GQSSRTR | 585 | QQYGDSQLFT | 586 |
| 1124 | | RATLSCRASQSVSSSYL | | YLA | | | | | |
| | | AWYQQKPGQAPRLLIY | | | | | | | |
| | | GQSSRTRGIPDRFSGS | | | | | | | |
| | | GSGTDFTLTISRLEPED | | | | | | | |
| | | FAVYYCQQYGDSQLFT | | | | | | | |
| | | FGQGTKLEIK | | | | | | | |
| mAb11- | VH | EVQLVQSGAEVKKPGE | 587 | TSWIV | 588 | MIDPSDSYTSYSPSFQ | 589 | LHYYNSEEFDV | 590 |
| 1125 | | SLRISCKGSGYSFSTSW | | | | G | | | |
| | | IVWVRQMPGKGLEWM | | | | | | | |
| | | GMIDPSDSYTSYSPSFQ | | | | | | | |
| | | GHVTISADKSISTAYLQ | | | | | | | |
| | | WSSLKASDTAMYYCAR | | | | | | | |
| | | LHYYNSEEFDVWGQGT | | | | | | | |
| | | LVTVSS | | | | | | | |
| mAb11- | VL | EIVLTQSPGTLSLSPGE | 591 | RASQSVSSS | 592 | GQSSRTR | 593 | QQYGDSQLFT | 594 |
| 1125 | | RATLSCRASQSVSSSYL | | YLA | | | | | |
| | | AWYQQKPGQAPRLLIY | | | | | | | |
| | | GQSSRTRGIPDRFSGS | | | | | | | |
| | | GSGTDFTLTISRLEPED | | | | | | | |
| | | FAVYYCQQYGDSQLFT | | | | | | | |
| | | FGQGTKLEIK | | | | | | | |
| mAb11- | VH | EVQLVQSGAEVKKPGE | 595 | TSWIV | 596 | MIDPSDSFTSYSPSFQ | 597 | LHYYNSEEFDV | 598 |
| 1127 | | SLRISCKGSGYSFSTSW | | | | G | | | |
| | | IVWVRQMPGKGLEWM | | | | | | | |
| | | GMIDPSDSFTSYSPSFQ | | | | | | | |
| | | GHVTISADKSISTAYLQ | | | | | | | |
| | | WSSLKASDTAMYYCAR | | | | | | | |
| | | LHYYNSEEFDVWGQGT | | | | | | | |
| | | LVTVSS | | | | | | | |
| mAb11- | VL | EIVLTQSPGTLSLSPGE | 599 | RASQSVSSS | 600 | GASSRAR | 601 | QQYGDSRLFT | 602 |
| 1127 | | RATLSCRASQSVSSSYL | | YLA | | | | | |
| | | AWYQQKPGQAPRLLIY | | | | | | | |
| | | GASSRARGIPDRFSGS | | | | | | | |
| | | GSGTDFTLTISRLEPED | | | | | | | |
| | | FAVYYCQQYGDSRLFT | | | | | | | |
| | | FGQGTKLEIK | | | | | | | |
| mAb11- | VH | EVQLVQSGAEVKKPGE | 603 | TSWIS | 604 | MIDPSDSYTSYSPSFQ | 605 | LHYYNSEEFDV | 606 |
| 1128 | | SLRISCKGSGYSFSTSW | | | | G | | | |
| | | ISWVRQMPGKGLEWM | | | | | | | |
| | | GMIDPSDSYTSYSPSFQ | | | | | | | |
| | | GHVTISADKSISTAYLQ | | | | | | | |
| | | WSSLKASDTAMYYCAR | | | | | | | |
| | | LHYYNSEEFDVWGQGT | | | | | | | |
| | | MVTVSS | | | | | | | |
| mAb11- | VL | EIVLTQSPGTLSLSPGE | 607 | RASQSVSSS | 608 | GASSRAR | 609 | QQYGDSRLFT | 610 |
| 1128 | | RATLSCRASQSVSSSYL | | YLA | | | | | |
| | | AWYQQKPGQAPRLLIY | | | | | | | |
| | | GASSRARGIPDRFSGS | | | | | | | |
| | | GSGTDFTLTISRLEPED | | | | | | | |
| | | FAVYYCQQYGDSRLFT | | | | | | | |
| | | FGQGTKLEIK | | | | | | | |
| mAb11- | VH | EVQLVQSGAEVKKPGE | 611 | TSWIS | 612 | MIDPSDSFTSYSPSFQ | 613 | LHYYHSEEFDV | 614 |
| 1129 | | SLRISCKGSGYSFTTSW | | | | G | | | |
| | | ISWVRQMPGKGLEWM | | | | | | | |
| | | GMIDPSDSFTSYSPSFQ | | | | | | | |
| | | GHVTISADKSISTAYLQ | | | | | | | |
| | | WSSLKASDTAMYYCAR | | | | | | | |
| | | LHYYHSEEFDVWGQGT | | | | | | | |
| | | MVTVSS | | | | | | | |
| mAb11- | VL | EIVLTQSPGTLSLSPGE | 615 | RASQSVSSS | 616 | GASSRAR | 617 | QQYGDSRLFT | 618 |
| 1129 | | RATLSCRASQSVSSSYL | | YLA | | | | | |
| | | AWYQQKPGQAPRLLIY | | | | | | | |
| | | GASSRARGIPDRFSGS | | | | | | | |
| | | GSGTDFTLTISRLEPED | | | | | | | |
| | | FAVYYCQQYGDSRLFT | | | | | | | |
| | | FGQGTKLEIK | | | | | | | |
| mAb11- | VH | EVQLVQSGAEVKKPGE | 619 | TSWIS | 620 | MIDPSDSYTSYSPSFQ | 621 | LHYYHSEEFDV | 622 |
| 1130 | | SLRISCKGSGYSFTTSW | | | | G | | | |
| | | ISWVRQMPGKGLEWM | | | | | | | |
| | | GMIDPSDSYTSYSPSFQ | | | | | | | |
| | | GHVTISADKSISTAYLQ | | | | | | | |
| | | WSSLKASDTAMYYCAR | | | | | | | |
| | | LHYYHSEEFDVWGQGT | | | | | | | |
| | | LVTVSS | | | | | | | |
| mAb11- | VL | EIVLTQSPGTLSLSPGE | 623 | RASQSVSSS | 624 | GASSRAR | 625 | QQYGDSRLFT | 626 |
| 1130 | | RATLSCRASQSVSSSYL | | YLA | | | | | |
| | | AWYQQKPGQAPRLLIY | | | | | | | |
| | | GASSRARGIPDRFSGS | | | | | | | |
| | | GSGTDFTLTISRLEPED | | | | | | | |
| | | FAVYYCQQYGDSRLFT | | | | | | | |
| | | FGQGTKLEIK | | | | | | | |
| mAb11- | VH | EVQLVQSGAEVKKPGE | 627 | TSWIS | 628 | MIDPSDSYTSYSPSFQ | 629 | LHYYHSEEFDV | 630 |
| 1131 | | SLRISCKGSGYSFTTSW | | | | G | | | |
| | | ISWVRQMPGKGLEWM | | | | | | | |
| | | GMIDPSDSYTSYSPSFQ | | | | | | | |
| | | GHVTISADKSISTAYLQ | | | | | | | |
| | | WSSLKASDTAMYYCAR | | | | | | | |
| | | LHYYHSEEFDVWGQGT | | | | | | | |
| | | MVTVSS | | | | | | | |
| mAb11- | VL | EIVLTQSPGTLSLSPGE | 631 | RASQSVSSS | 632 | GASSRAR | 633 | QQYGDSRLFT | 634 |
| 1131 | | RATLSCRASQSVSSSYL | | YLA | | | | | |
| | | AWYQQKPGQAPRLLIY | | | | | | | |
| | | GASSRARGIPDRFSGS | | | | | | | |
| | | GSGTDFTLTISRLEPED | | | | | | | |
| | | FAVYYCQQYGDSRLFT | | | | | | | |
| | | FGQGTKLEIK | | | | | | | |
| mAb11- | VH | EVQLVQSGAEVKKPGE | 635 | TSWIS | 636 | MIDPSDSFTSYSPSFQ | 637 | LHYYNSEEFDV | 638 |
| 1132 | | SLRISCKGSGYSFSTSW | | | | G | | | |
| | | ISWVRQMPGKGLEWM | | | | | | | |
| | | GMIDPSDSFTSYSPSFQ | | | | | | | |
| | | GHVTISADKSISTAYLQ | | | | | | | |
| | | WSSLKASDTAMYYCAR | | | | | | | |
| | | LHYYNSEEFDVWGQGT | | | | | | | |
| | | LVTVSS | | | | | | | |
| mAb11- | VL | EIVLTQSPGTLSLSPGE | 639 | RASQSVSSS | 640 | GASSRAR | 641 | QQYGDSRLFT | 642 |
| 1132 | | RATLSCRASQSVSSSYL | | YLA | | | | | |
| | | AWYQQKPGQAPRLLIY | | | | | | | |
| | | GASSRARGIPDRFSGS | | | | | | | |
| | | GSGTDFTLTISRLEPED | | | | | | | |
| | | FAVYYCQQYGDSRLFT | | | | | | | |
| | | FGQGTKLEIK | | | | | | | |
| mAb11- | VH | EVQLVQSGAEVKKPGE | 643 | TSWIV | 644 | MIDPSDSYTSYSPSFQ | 645 | LHYYNSEEFDV | 646 |
| 1133 | | SLRISCKGSGYSFSTSW | | | | G | | | |
| | | IVWVRQMPGKGLEWM | | | | | | | |
| | | GMIDPSDSYTSYSPSFQ | | | | | | | |
| | | GHVTISADKSISTAYLQ | | | | | | | |
| | | WSSLKASDTAMYYCAR | | | | | | | |
| | | LHYYNSEEFDVWGQGT | | | | | | | |
| | | LVTVSS | | | | | | | |
| mAb11- | VL | EIVLTQSPGTLSLSPGE | 647 | RASQSVSSS | 648 | GASSRAR | 649 | QQYGDSRLFT | 650 |
| 1133 | | RATLSCRASQSVSSSYL | | YLA | | | | | |
| | | AWYQQKPGQAPRLLIY | | | | | | | |
| | | GASSRARGIPDRFSGS | | | | | | | |
| | | GSGTDFTLTISRLEPED | | | | | | | |
| | | FAVYYCQQYGDSRLFT | | | | | | | |
| | | FGQGTKLEIK | | | | | | | |
| mAb11- | VH | EVQLVQSGAEVKKPGE | 651 | TSWIV | 652 | MIDPSDSFTSYSPSFQ | 653 | LHYYNSEEFDV | 654 |
| 1416 | | SLRISCKGSGYSFSTSW | | | | G | | | |
| | | IVWVRQMPGKGLEWM | | | | | | | |
| | | GMIDPSDSFTSYSPSFQ | | | | | | | |
| | | GHVTISADKSISTAYLQ | | | | | | | |
| | | WSSLKASDTAMYYCAR | | | | | | | |
| | | LHYYNSEEFDVWGQGT | | | | | | | |
| | | LVTVSS | | | | | | | |
| mAb11- | VL | EIVLTQSPGTLSLSPGE | 655 | RASQSVSSS | 656 | GQSSRTR | 657 | QQFGDSRLFT | 658 |
| 1416 | | RATLSCRASQSVSSSYL | | YLA | | | | | |
| | | AWYQQKPGQAPRLLIY | | | | | | | |
| | | GQSSRTRGIPDRFSGS | | | | | | | |
| | | GSGTDFTLTISRLEPED | | | | | | | |
| | | FAVYYCQQFGDSRLFT | | | | | | | |
| | | FGQGTKLEIK | | | | | | | |
| mAb11- | VH | EVQLVQSGAEVKKPGE | 659 | TSWIS | 660 | MIDPSDSYTSYSPSFQ | 661 | LHYYNSEEFDV | 662 |
| 1417 | | SLRISCKGSGYSFSTSW | | | | G | | | |
| | | ISWVRQMPGKGLEWM | | | | | | | |
| | | GMIDPSDSYTSYSPSFQ | | | | | | | |
| | | GHVTISADKSISTAYLQ | | | | | | | |
| | | WSSLKASDTAMYYCAR | | | | | | | |
| | | LHYYNSEEFDVWGQGT | | | | | | | |
| | | MVTVSS | | | | | | | |
| mAb11- | VL | EIVLTQSPGTLSLSPGE | 663 | RASQSVSSS | 664 | GQSSRTR | 665 | QQFGDSRLFT | 666 |
| 1417 | | RATLSCRASQSVSSSYL | | YLA | | | | | |
| | | AWYQQKPGQAPRLLIY | | | | | | | |
| | | GQSSRTRGIPDRFSGS | | | | | | | |
| | | GSGTDFTLTISRLEPED | | | | | | | |
| | | FAVYYCQQFGDSRLFT | | | | | | | |
| | | FGQGTKLEIK | | | | | | | |
| mAb11- | VH | EVQLVQSGAEVKKPGE | 667 | TSWIS | 668 | MIDPSDSFTSYSPSFQ | 669 | LHYYHSEEFDV | 670 |
| 1418 | | SLRISCKGSGYSFTTSW | | | | G | | | |
| | | ISWVRQMPGKGLEWM | | | | | | | |
| | | GMIDPSDSFTSYSPSFQ | | | | | | | |
| | | GHVTISADKSISTAYLQ | | | | | | | |
| | | WSSLKASDTAMYYCAR | | | | | | | |
| | | LHYYHSEEFDVWGQGT | | | | | | | |
| | | MVTVSS | | | | | | | |
| mAb11- | VL | EIVLTQSPGTLSLSPGE | 671 | RASQSVSSS | 672 | GQSSRTR | 673 | QQFGDSRLFT | 674 |
| 1418 | | RATLSCRASQSVSSSYL | | YLA | | | | | |
| | | AWYQQKPGQAPRLLIY | | | | | | | |
| | | GQSSRTRGIPDRFSGS | | | | | | | |
| | | GSGTDFTLTISRLEPED | | | | | | | |
| | | FAVYYCQQFGDSRLFT | | | | | | | |
| | | FGQGTKLEIK | | | | | | | |
| mAb11- | VH | EVQLVQSGAEVKKPGE | 675 | TSWIS | 676 | MIDPSDSYTSYSPSFQ | 677 | LHYYHSEEFDV | 678 |
| 1419 | | SLRISCKGSGYSFTTSW | | | | G | | | |
| | | ISWVRQMPGKGLEWM | | | | | | | |
| | | GMIDPSDSYTSYSPSFQ | | | | | | | |
| | | GHVTISADKSISTAYLQ | | | | | | | |
| | | WSSLKASDTAMYYCAR | | | | | | | |
| | | LHYYHSEEFDVWGQGT | | | | | | | |
| | | LVTVSS | | | | | | | |
| mAb11- | VL | EIVLTQSPGTLSLSPGE | 679 | RASQSVSSS | 680 | GQSSRTR | 681 | QQFGDSRLFT | 682 |
| 1419 | | RATLSCRASQSVSSSYL | | YLA | | | | | |
| | | AWYQQKPGQAPRLLIY | | | | | | | |
| | | GQSSRTRGIPDRFSGS | | | | | | | |
| | | GSGTDFTLTISRLEPED | | | | | | | |
| | | FAVYYCQQFGDSRLFT | | | | | | | |
| | | FGQGTKLEIK | | | | | | | |
| mAb11- | VH | EVQLVQSGAEVKKPGE | 683 | TSWIS | 684 | MIDPSDSYTSYSPSFQ | 685 | LHYYHSEEFDV | 686 |
| 1420 | | SLRISCKGSGYSFTTSW | | | | G | | | |
| | | ISWVRQMPGKGLEWM | | | | | | | |
| | | GMIDPSDSYTSYSPSFQ | | | | | | | |
| | | GHVTISADKSISTAYLQ | | | | | | | |
| | | WSSLKASDTAMYYCAR | | | | | | | |
| | | LHYYHSEEFDVWGQGT | | | | | | | |
| | | MVTVSS | | | | | | | |
| mAb11- | VL | EIVLTQSPGTLSLSPGE | 687 | RASQSVSSS | 688 | GQSSRTR | 689 | QQFGDSRLFT | 690 |
| 1420 | | RATLSCRASQSVSSSYL | | YLA | | | | | |
| | | AWYQQKPGQAPRLLIY | | | | | | | |
| | | GQSSRTRGIPDRFSGS | | | | | | | |
| | | GSGTDFTLTISRLEPED | | | | | | | |
| | | FAVYYCQQFGDSRLFT | | | | | | | |
| | | FGQGTKLEIK | | | | | | | |
| mAb11- | VH | EVQLVQSGAEVKKPGE | 691 | TSWIS | 692 | MIDPSDSFTSYSPSFQ | 693 | LHYYNSEEFDV | 694 |
| 1421 | | SLRISCKGSGYSFSTSW | | | | G | | | |
| | | ISWVRQMPGKGLEWM | | | | | | | |
| | | GMIDPSDSFTSYSPSFQ | | | | | | | |
| | | GHVTISADKSISTAYLQ | | | | | | | |
| | | WSSLKASDTAMYYCAR | | | | | | | |
| | | LHYYNSEEFDVWGQGT | | | | | | | |
| | | LVTVSS | | | | | | | |
| mAb11- | VL | EIVLTQSPGTLSLSPGE | 695 | RASQSVSSS | 696 | GQSSRTR | 697 | QQFGDSRLFT | 698 |
| 1421 | | RATLSCRASQSVSSSYL | | YLA | | | | | |
| | | AWYQQKPGQAPRLLIY | | | | | | | |
| | | GQSSRTRGIPDRFSGS | | | | | | | |
| | | GSGTDFTLTISRLEPED | | | | | | | |
| | | FAVYYCQQFGDSRLFT | | | | | | | |
| | | FGQGTKLEIK | | | | | | | |
| mAb11- | VH | EVQLVQSGAEVKKPGE | 699 | TSWIV | 700 | MIDPSDSYTSYSPSFQ | 701 | LHYYNSEEFDV | 702 |
| 1422 | | SLRISCKGSGYSFSTSW | | | | G | | | |
| | | IVWVRQMPGKGLEWM | | | | | | | |
| | | GMIDPSDSYTSYSPSFQ | | | | | | | |
| | | GHVTISADKSISTAYLQ | | | | | | | |
| | | WSSLKASDTAMYYCAR | | | | | | | |
| | | LHYYNSEEFDVWGQGT | | | | | | | |
| | | LVTVSS | | | | | | | |
| mAb11- | VL | EIVLTQSPGTLSLSPGE | 703 | RASQSVSSS | 704 | GQSSRTR | 705 | QQFGDSRLFT | 706 |
| 1422 | | RATLSCRASQSVSSSYL | | YLA | | | | | |
| | | AWYQQKPGQAPRLLIY | | | | | | | |
| | | GQSSRTRGIPDRFSGS | | | | | | | |
| | | GSGTDFTLTISRLEPED | | | | | | | |
| | | FAVYYCQQFGDSRLFT | | | | | | | |
| | | FGQGTKLEIK | | | | | | | |
| mAb11- | VH | EVQLVQSGAEVKKPGE | 707 | TSWIV | 708 | MIDPSDSFTSYSPSFQ | 709 | LHYYNSEEFDV | 710 |
| 1431 | | SLRISCKGSGYSFSTSW | | | | G | | | |
| | | IVWVRQMPGKGLEWM | | | | | | | |
| | | GMIDPSDSFTSYSPSFQ | | | | | | | |
| | | GHVTISADKSISTAYLQ | | | | | | | |
| | | WSSLKASDTAMYYCAR | | | | | | | |
| | | LHYYNSEEFDVWGQGT | | | | | | | |
| | | LVTVSS | | | | | | | |
| mAb11- | VL | EIVLTQSPGTLSLSPGE | 711 | RASQSVSSS | 712 | GQSSRTR | 713 | QQFGSSQLFT | 714 |
| 1431 | | RATLSCRASQSVSSSYL | | YLA | | | | | |
| | | AWYQQKPGQAPRLLIY | | | | | | | |
| | | GQSSRTRGIPDRFSGS | | | | | | | |
| | | GSGTDFTLTISRLEPED | | | | | | | |
| | | FAVYYCQQFGSSQLFT | | | | | | | |
| | | FGQGTKLEIK | | | | | | | |
| mAb11- | VH | EVQLVQSGAEVKKPGE | 715 | TSWIV | 716 | MIDPSDSFTSYSPSFQ | 717 | LHYYNSEEFDV | 718 |
| 1432 | | SLRISCKGSGYSFSTSW | | | | G | | | |
| | | IVWVRQMPGKGLEWM | | | | | | | |
| | | GMIDPSDSFTSYSPSFQ | | | | | | | |
| | | GHVTISADKSISTAYLQ | | | | | | | |
| | | WSSLKASDTAMYYCAR | | | | | | | |
| | | LHYYNSEEFDVWGQGT | | | | | | | |
| | | LVTVSS | | | | | | | |
| mAb11- | VL | EIVLTQSPGTLSLSPGE | 719 | RASQSVSSS | 720 | GQSSRTR | 721 | QQFGESQLFT | 722 |
| 1432 | | RATLSCRASQSVSSSYL | | YLA | | | | | |
| | | AWYQQKPGQAPRLLIY | | | | | | | |
| | | GQSSRTRGIPDRFSGS | | | | | | | |
| | | GSGTDFTLTISRLEPED | | | | | | | |
| | | FAVYYCQQFGESQLFT | | | | | | | |
| | | FGQGTKLEIK | | | | | | | |
| mAb11- | VH | EVQLVQSGAEVKKPGE | 723 | TSWIV | 724 | MIDPSDSFTSYSPSFQ | 725 | LHYYNSEEFDV | 726 |
| 1433 | | SLRISCKGSGYSFSTSW | | | | G | | | |
| | | IVWVRQMPGKGLEWM | | | | | | | |
| | | GMIDPSDSFTSYSPSFQ | | | | | | | |
| | | GHVTISADKSISTAYLQ | | | | | | | |
| | | WSSLKASDTAMYYCAR | | | | | | | |
| | | LHYYNSEEFDVWGQGT | | | | | | | |
| | | LVTVSS | | | | | | | |
| mAb11- | VL | EIVLTQSPGTLSLSPGE | 727 | RASQSVSSS | 728 | GQSSRTR | 729 | QQFGNSQLFT | 730 |
| 1433 | | RATLSCRASQSVSSSYL | | YLA | | | | | |
| | | AWYQQKPGQAPRLLIY | | | | | | | |
| | | GQSSRTRGIPDRFSGS | | | | | | | |
| | | GSGTDFTLTISRLEPED | | | | | | | |
| | | FAVYYCQQFGNSQLFT | | | | | | | |
| | | FGQGTKLEIK | | | | | | | |
| mAb11- | VH | EVQLVQSGAEVKKPGE | 731 | TSWIV | 732 | MIDPSDSFTSYSPSFQ | 733 | LHYYNSEEFDV | 734 |
| 1434 | | SLRISCKGSGYSFSTSW | | | | G | | | |
| | | IVWVRQMPGKGLEWM | | | | | | | |
| | | GMIDPSDSFTSYSPSFQ | | | | | | | |
| | | GHVTISADKSISTAYLQ | | | | | | | |
| | | WSSLKASDTAMYYCAR | | | | | | | |
| | | LHYYNSEEFDVWGQGT | | | | | | | |
| | | LVTVSS | | | | | | | |
| mAb11- | VL | EIVLTQSPGTLSLSPGE | 735 | RASQSVSSS | 736 | GQSSRTR | 737 | QQFGQSQLFT | 738 |
| 1434 | | RATLSCRASQSVSSSYL | | YLA | | | | | |
| | | AWYQQKPGQAPRLLIY | | | | | | | |
| | | GQSSRTRGIPDRFSGS | | | | | | | |
| | | GSGTDFTLTISRLEPED | | | | | | | |
| | | FAVYYCQQFGQSQLFT | | | | | | | |
| | | FGQGTKLEIK | | | | | | | |
| mAb11- | VH | EVQLVQSGAEVKKPGE | 739 | TSWIV | 740 | MIDPSDSFTSYSPSFQ | 741 | LHYYNSEEFDV | 742 |
| 1435 | | SLRISCKGSGYSFSTSW | | | | G | | | |
| | | IVWVRQMPGKGLEWM | | | | | | | |
| | | GMIDPSDSFTSYSPSFQ | | | | | | | |
| | | GHVTISADKSISTAYLQ | | | | | | | |
| | | WSSLKASDTAMYYCAR | | | | | | | |
| | | LHYYNSEEFDVWGQGT | | | | | | | |
| | | LVTVSS | | | | | | | |
| mAb11- | VL | EIVLTQSPGTLSLSPGE | 743 | RASQSVSSS | 744 | GQSSRTR | 745 | QQFGDAQLFT | 746 |
| 1435 | | RATLSCRASQSVSSSYL | | YLA | | | | | |
| | | AWYQQKPGQAPRLLIY | | | | | | | |
| | | GQSSRTRGIPDRFSGS | | | | | | | |
| | | GSGTDFTLTISRLEPED | | | | | | | |
| | | FAVYYCQQFGDAQLFT | | | | | | | |
| | | FGQGTKLEIK | | | | | | | |
| mAb11- | VH | EVQLVQSGAEVKKPGE | 747 | TSWIV | 748 | MIDPSDSFTSYSPSFQ | 749 | LHYYNSEEFDV | 750 |
| 1436 | | SLRISCKGSGYSFSTSW | | | | G | | | |
| | | IVWVRQMPGKGLEWM | | | | | | | |
| | | GMIDPSDSFTSYSPSFQ | | | | | | | |
| | | GHVTISADKSISTAYLQ | | | | | | | |
| | | WSSLKASDTAMYYCAR | | | | | | | |
| | | LHYYNSEEFDVWGQGT | | | | | | | |
| | | LVTVSS | | | | | | | |
| mAb11- | VL | EIVLTQSPGTLSLSPGE | 751 | RASQSVSSS | 752 | GQSSRTR | 753 | QQFGDTQLFT | 754 |
| 1436 | | RATLSCRASQSVSSSYL | | YLA | | | | | |
| | | AWYQQKPGQAPRLLIY | | | | | | | |
| | | GQSSRTRGIPDRFSGS | | | | | | | |
| | | GSGTDFTLTISRLEPED | | | | | | | |
| | | FAVYYCQQFGDTQLFT | | | | | | | |
| | | FGQGTKLEIK | | | | | | | |
| mAb11- | VH | EVQLVQSGAEVKKPGE | 755 | TSWIV | 756 | MIDPSDSFTSYSPSFQ | 757 | LHYYNSEEFDV | 758 |
| 1437 | | SLRISCKGSGYSFSTSW | | | G | | | | |
| | | IVWVRQMPGKGLEWM | | | | | | | |
| | | GMIDPSDSFTSYSPSFQ | | | | | | | |
| | | GHVTISADKSISTAYLQ | | | | | | | |
| | | WSSLKASDTAMYYCAR | | | | | | | |
| | | LHYYNSEEFDVWGQGT | | | | | | | |
| | | LVTVSS | | | | | | | |
| mAb11- | VL | EIVLTQSPGTLSLSPGE | 759 | RASQSVSSS | 760 | GQSSRTR | 761 | QQFGDNQLFT | 762 |
| 1437 | | RATLSCRASQSVSSSYL | | YLA | | | | | |
| | | AWYQQKPGQAPRLLIY | | | | | | | |
| | | GQSSRTRGIPDRFSGS | | | | | | | |
| | | GSGTDFTLTISRLEPED | | | | | | | |
| | | FAVYYCQQFGDNQLFT | | | | | | | |
| | | FGQGTKLEIK | | | | | | | |
| mAb11- | VH | EVQLVQSGAEVKKPGE | 763 | TSWIS | 764 | MIDPSDSYTSYSPSFQ | 765 | LHYYNSEEFDV | 766 |
| 1439 | | SLRISCKGSGYSFSTSW | | | | G | | | |
| | | ISWVRQMPGKGLEWM | | | | | | | |
| | | GMIDPSDSYTSYSPSFQ | | | | | | | |
| | | GHVTISADKSISTAYLQ | | | | | | | |
| | | WSSLKASDTAMYYCAR | | | | | | | |
| | | LHYYNSEEFDVWGQGT | | | | | | | |
| | | MVTVSS | | | | | | | |
| mAb11- | VL | EIVLTQSPGTLSLSPGE | 767 | RASQSVSSS | 768 | GQSSRTR | 769 | QQFGSSQLFT | 770 |
| 1439 | | RATLSCRASQSVSSSYL | | YLA | | | | | |
| | | AWYQQKPGQAPRLLIY | | | | | | | |
| | | GQSSRTRGIPDRFSGS | | | | | | | |
| | | GSGTDFTLTISRLEPED | | | | | | | |
| | | FAVYYCQQFGSSQLFT | | | | | | | |
| | | FGQGTKLEIK | | | | | | | |
| mAb11- | VH | EVQLVQSGAEVKKPGE | 771 | TSWIS | 772 | MIDPSDSFTSYSPSFQ | 773 | LHYYHSEEFDV | 774 |
| 1440 | | SLRISCKGSGYSFTTSW | | | | G | | | |
| | | ISWVRQMPGKGLEWM | | | | | | | |
| | | GMIDPSDSFTSYSPSFQ | | | | | | | |
| | | GHVTISADKSISTAYLQ | | | | | | | |
| | | WSSLKASDTAMYYCAR | | | | | | | |
| | | LHYYHSEEFDVWGQGT | | | | | | | |
| | | MVTVSS | | | | | | | |
| mAb11- | VL | EIVLTQSPGTLSLSPGE | 775 | RASQSVSSS | 776 | GQSSRTR | 777 | QQFGSSQLFT | 778 |
| 1440 | | RATLSCRASQSVSSSYL | | YLA | | | | | |
| | | AWYQQKPGQAPRLLIY | | | | | | | |
| | | GQSSRTRGIPDRFSGS | | | | | | | |
| | | GSGTDFTLTISRLEPED | | | | | | | |
| | | FAVYYCQQFGSSQLFT | | | | | | | |
| | | FGQGTKLEIK | | | | | | | |
| mAb11- | VH | EVQLVQSGAEVKKPGE | 779 | TSWIS | 780 | MIDPSDSYTSYSPSFQ | 781 | LHYYHSEEFDV | 782 |
| 1441 | | SLRISCKGSGYSFTTSW | | | | G | | | |
| | | ISWVRQMPGKGLEWM | | | | | | | |
| | | GMIDPSDSYTSYSPSFQ | | | | | | | |
| | | GHVTISADKSISTAYLQ | | | | | | | |
| | | WSSLKASDTAMYYCAR | | | | | | | |
| | | LHYYHSEEFDVWGQGT | | | | | | | |
| | | LVTVSS | | | | | | | |
| mAb11- | VL | EIVLTQSPGTLSLSPGE | 783 | RASQSVSSS | 784 | GQSSRTR | 785 | QQFGSSQLFT | 786 |
| 1441 | | RATLSCRASQSVSSSYL | | YLA | | | | | |
| | | AWYQQKPGQAPRLLIY | | | | | | | |
| | | GQSSRTRGIPDRFSGS | | | | | | | |
| | | GSGTDFTLTISRLEPED | | | | | | | |
| | | FAVYYCQQFGSSQLFT | | | | | | | |
| | | FGQGTKLEIK | | | | | | | |
| mAb11- | VH | EVQLVQSGAEVKKPGE | 787 | TSWIS | 788 | MIDPSDSYTSYSPSFQ | 789 | LHYYHSEEFDV | 790 |
| 1442 | | SLRISCKGSGYSFTTSW | | | | G | | | |
| | | ISWVRQMPGKGLEWM | | | | | | | |
| | | GMIDPSDSYTSYSPSFQ | | | | | | | |
| | | GHVTISADKSISTAYLQ | | | | | | | |
| | | WSSLKASDTAMYYCAR | | | | | | | |
| | | LHYYHSEEFDVWGQGT | | | | | | | |
| | | MVTVSS | | | | | | | |
| mAb11- | VL | EIVLTQSPGTLSLSPGE | 791 | RASQSVSSS | 792 | GQSSRTR | 793 | QQFGSSQLFT | 794 |
| 1442 | | RATLSCRASQSVSSSYL | | YLA | | | | | |
| | | AWYQQKPGQAPRLLIY | | | | | | | |
| | | GQSSRTRGIPDRFSGS | | | | | | | |
| | | GSGTDFTLTISRLEPED | | | | | | | |
| | | FAVYYCQQFGSSQLFT | | | | | | | |
| | | FGQGTKLEIK | | | | | | | |
| mAb11- | VH | EVQLVQSGAEVKKPGE | 795 | TSWIS | 796 | MIDPSDSFTSYSPSFQ | 797 | LHYYNSEEFDV | 798 |
| 1443 | | SLRISCKGSGYSFSTSW | | | | G | | | |
| | | ISWVRQMPGKGLEWM | | | | | | | |
| | | GMIDPSDSFTSYSPSFQ | | | | | | | |
| | | GHVTISADKSISTAYLQ | | | | | | | |
| | | WSSLKASDTAMYYCAR | | | | | | | |
| | | LHYYNSEEFDVWGQGT | | | | | | | |
| | | LVTVSS | | | | | | | |
| mAb11- | VL | EIVLTQSPGTLSLSPGE | 799 | RASQSVSSS | 800 | GQSSRTR | 801 | QQFGSSQLFT | 802 |
| 1443 | | RATLSCRASQSVSSSYL | | YLA | | | | | |
| | | AWYQQKPGQAPRLLIY | | | | | | | |
| | | GQSSRTRGIPDRFSGS | | | | | | | |
| | | GSGTDFTLTISRLEPED | | | | | | | |
| | | FAVYYCQQFGSSQLFT | | | | | | | |
| | | FGQGTKLEIK | | | | | | | |
| mAb11- | VH | EVQLVQSGAEVKKPGE | 803 | TSWIV | 804 | MIDPSDSYTSYSPSFQ | 805 | LHYYNSEEFDV | 806 |
| 1444 | | SLRISCKGSGYSFSTSW | | | | G | | | |
| | | IVWVRQMPGKGLEWM | | | | | | | |
| | | GMIDPSDSYTSYSPSFQ | | | | | | | |
| | | GHVTISADKSISTAYLQ | | | | | | | |
| | | WSSLKASDTAMYYCAR | | | | | | | |
| | | LHYYNSEEFDVWGQGT | | | | | | | |
| | | LVTVSS | | | | | | | |
| mAb11- | VL | EIVLTQSPGTLSLSPGE | 807 | RASQSVSSS | 808 | GQSSRTR | 809 | QQFGSSQLFT | 810 |
| 1444 | | RATLSCRASQSVSSSYL | | YLA | | | | | |
| | | AWYQQKPGQAPRLLIY | | | | | | | |
| | | GQSSRTRGIPDRFSGS | | | | | | | |
| | | GSGTDFTLTISRLEPED | | | | | | | |
| | | FAVYYCQQFGSSQLFT | | | | | | | |
| | | FGQGTKLEIK | | | | | | | |
| mAb11- | VH | EVQLVQSGAEVKKPGE | 811 | TSWIV | 812 | MIDPSDSYTSYSPSFQ | 813 | LHYYNSEEFDV | 814 |
| 1445 | | SLRISCKGSGYSFSTSW | | | | G | | | |
| | | IVWVRQMPGKGLEWM | | | | | | | |
| | | GMIDPSDSYTSYSPSFQ | | | | | | | |
| | | GHVTISADKSISTAYLQ | | | | | | | |
| | | WSSLKASDTAMYYCAR | | | | | | | |
| | | LHYYNSEEFDVWGQGT | | | | | | | |
| | | MVTVSS | | | | | | | |
| mAb11- | VL | EIVLTQSPGTLSLSPGE | 815 | RASQSVSSS | 816 | GQSSRTR | 817 | QQFGSSQLFT | 818 |
| 1445 | | RATLSCRASQSVSSSYL | | YLA | | | | | |
| | | AWYQQKPGQAPRLLIY | | | | | | | |
| | | GQSSRTRGIPDRFSGS | | | | | | | |
| | | GSGTDFTLTISRLEPED | | | | | | | |
| | | FAVYYCQQFGSSQLFT | | | | | | | |
| | | FGQGTKLEIK | | | | | | | |
| mAb11- | VH | EVQLVQSGAEVKKPGE | 819 | TSWIS | 820 | MIDPSDSYTSYSPSFQ | 821 | LHYYNSEEFDV | 822 |
| 1446 | | SLRISCKGSGYSFSTSW | | | | G | | | |
| | | ISWVRQMPGKGLEWM | | | | | | | |
| | | GMIDPSDSYTSYSPSFQ | | | | | | | |
| | | GHVTISADKSISTAYLQ | | | | | | | |
| | | WSSLKASDTAMYYCAR | | | | | | | |
| | | LHYYNSEEFDVWGQGT | | | | | | | |
| | | MVTVSS | | | | | | | |
| mAb11- | VL | EIVLTQSPGTLSLSPGE | 823 | RASQSVSSS | 824 | GQSSRTR | 825 | QQFGESQLFT | 826 |
| 1446 | | RATLSCRASQSVSSSYL | | YLA | | | | | |
| | | AWYQQKPGQAPRLLIY | | | | | | | |
| | | GQSSRTRGIPDRFSGS | | | | | | | |
| | | GSGTDFTLTISRLEPED | | | | | | | |
| | | FAVYYCQQFGESQLFT | | | | | | | |
| | | FGQGTKLEIK | | | | | | | |
| mAb11- | VH | EVQLVQSGAEVKKPGE | 827 | TSWIS | 828 | MIDPSDSFTSYSPSFQ | 829 | LHYYHSEEFDV | 830 |
| 1447 | | SLRISCKGSGYSFTTSW | | | | G | | | |
| | | ISWVRQMPGKGLEWM | | | | | | | |
| | | GMIDPSDSFTSYSPSFQ | | | | | | | |
| | | GHVTISADKSISTAYLQ | | | | | | | |
| | | WSSLKASDTAMYYCAR | | | | | | | |
| | | LHYYHSEEFDVWGQGT | | | | | | | |
| | | MVTVSS | | | | | | | |
| mAb11- | VL | EIVLTQSPGTLSLSPGE | 831 | RASQSVSSS | 832 | GQSSRTR | 833 | QQFGESQLFT | 834 |
| 1447 | | RATLSCRASQSVSSSYL | | YLA | | | | | |
| | | AWYQQKPGQAPRLLIY | | | | | | | |
| | | GQSSRTRGIPDRFSGS | | | | | | | |
| | | GSGTDFTLTISRLEPED | | | | | | | |
| | | FAVYYCQQFGESQLFT | | | | | | | |
| | | FGQGTKLEIK | | | | | | | |
| mAb11- | VH | EVQLVQSGAEVKKPGE | 835 | TSWIS | 836 | MIDPSDSYTSYSPSFQ | 837 | LHYYHSEEFDV | 838 |
| 1448 | | SLRISCKGSGYSFTTSW | | | | G | | | |
| | | ISWVRQMPGKGLEWM | | | | | | | |
| | | GMIDPSDSYTSYSPSFQ | | | | | | | |
| | | GHVTISADKSISTAYLQ | | | | | | | |
| | | WSSLKASDTAMYYCAR | | | | | | | |
| | | LHYYHSEEFDVWGQGT | | | | | | | |
| | | LVTVSS | | | | | | | |
| mAb11- | VL | EIVLTQSPGTLSLSPGE | 839 | RASQSVSSS | 840 | GQSSRTR | 841 | QQFGESQLFT | 842 |
| 1448 | | RATLSCRASQSVSSSYL | | YLA | | | | | |
| | | AWYQQKPGQAPRLLIY | | | | | | | |
| | | GQSSRTRGIPDRFSGS | | | | | | | |
| | | GSGTDFTLTISRLEPED | | | | | | | |
| | | FAVYYCQQFGESQLFT | | | | | | | |
| | | FGQGTKLEIK | | | | | | | |
| mAb11- | VH | EVQLVQSGAEVKKPGE | 843 | TSWIS | 844 | MIDPSDSYTSYSPSFQ | 845 | LHYYHSEEFDV | 846 |
| 1449 | | SLRISCKGSGYSFTTSW | | | | G | | | |
| | | ISWVRQMPGKGLEWM | | | | | | | |
| | | GMIDPSDSYTSYSPSFQ | | | | | | | |
| | | GHVTISADKSISTAYLQ | | | | | | | |
| | | WSSLKASDTAMYYCAR | | | | | | | |
| | | LHYYHSEEFDVWGQGT | | | | | | | |
| | | MVTVSS | | | | | | | |
| mAb11- | VL | EIVLTQSPGTLSLSPGE | 847 | RASQSVSSS | 848 | GQSSRTR | 849 | QQFGESQLFT | 850 |
| 1449 | | RATLSCRASQSVSSSYL | | YLA | | | | | |
| | | AWYQQKPGQAPRLLIY | | | | | | | |
| | | GQSSRTRGIPDRFSGS | | | | | | | |
| | | GSGTDFTLTISRLEPED | | | | | | | |
| | | FAVYYCQQFGESQLFT | | | | | | | |
| | | FGQGTKLEIK | | | | | | | |
| mAb11- | VH | EVQLVQSGAEVKKPGE | 851 | TSWIS | 852 | MIDPSDSFTSYSPSFQ | 853 | LHYYNSEEFDV | 854 |
| 1450 | | SLRISCKGSGYSFSTSW | | | | G | | | |
| | | ISWVRQMPGKGLEWM | | | | | | | |
| | | GMIDPSDSFTSYSPSFQ | | | | | | | |
| | | GHVTISADKSISTAYLQ | | | | | | | |
| | | WSSLKASDTAMYYCAR | | | | | | | |
| | | LHYYNSEEFDVWGQGT | | | | | | | |
| | | LVTVSS | | | | | | | |
| mAb11- | VL | EIVLTQSPGTLSLSPGE | 855 | RASQSVSSS | 856 | GQSSRTR | 857 | QQFGESQLFT | 858 |
| 1450 | | RATLSCRASQSVSSSYL | | YLA | | | | | |
| | | AWYQQKPGQAPRLLIY | | | | | | | |
| | | GQSSRTRGIPDRFSGS | | | | | | | |
| | | GSGTDFTLTISRLEPED | | | | | | | |
| | | FAVYYCQQFGESQLFT | | | | | | | |
| | | FGQGTKLEIK | | | | | | | |
| mAb11- | VH | EVQLVQSGAEVKKPGE | 859 | TSWIV | 860 | MIDPSDSYTSYSPSFQ | 861 | LHYYNSEEFDV | 862 |
| 1451 | | SLRISCKGSGYSFSTSW | | | | G | | | |
| | | IVWVRQMPGKGLEWM | | | | | | | |
| | | GMIDPSDSYTSYSPSFQ | | | | | | | |
| | | GHVTISADKSISTAYLQ | | | | | | | |
| | | WSSLKASDTAMYYCAR | | | | | | | |
| | | LHYYNSEEFDVWGQGT | | | | | | | |
| | | LVTVSS | | | | | | | |
| mAb11- | VL | EIVLTQSPGTLSLSPGE | 863 | RASQSVSSS | 864 | GQSSRTR | 865 | QQFGESQLFT | 866 |
| 1451 | | RATLSCRASQSVSSSYL | | YLA | | | | | |
| | | AWYQQKPGQAPRLLIY | | | | | | | |
| | | GQSSRTRGIPDRFSGS | | | | | | | |
| | | GSGTDFTLTISRLEPED | | | | | | | |
| | | FAVYYCQQFGESQLFT | | | | | | | |
| | | FGQGTKLEIK | | | | | | | |
| mAb11- | VH | EVQLVQSGAEVKKPGE | 867 | TSWIV | 868 | MIDPSDSYTSYSPSFQ | 869 | LHYYNSEEFDV | 870 |
| 1452 | | SLRISCKGSGYSFSTSW | | | | G | | | |
| | | IVWVRQMPGKGLEWM | | | | | | | |
| | | GMIDPSDSYTSYSPSFQ | | | | | | | |
| | | GHVTISADKSISTAYLQ | | | | | | | |
| | | WSSLKASDTAMYYCAR | | | | | | | |
| | | LHYYNSEEFDVWGQGT | | | | | | | |
| | | MVTVSS | | | | | | | |
| mAb11- | VL | EIVLTQSPGTLSLSPGE | 871 | RASQSVSSS | 872 | GQSSRTR | 873 | QQFGESQLFT | 874 |
| 1452 | | RATLSCRASQSVSSSYL | | YLA | | | | | |
| | | AWYQQKPGQAPRLLIY | | | | | | | |
| | | GQSSRTRGIPDRFSGS | | | | | | | |
| | | GSGTDFTLTISRLEPED | | | | | | | |
| | | FAVYYCQQFGESQLFT | | | | | | | |
| | | FGQGTKLEIK | | | | | | | |
| mAb11- | VH | EVQLVQSGAEVKKPGE | 875 | TSWIS | 876 | MIDPSDSYTSYSPSFQ | 877 | LHYYNSEEFDV | 878 |
| 1453 | | SLRISCKGSGYSFSTSW | | | | G | | | |
| | | ISWVRQMPGKGLEWM | | | | | | | |
| | | GMIDPSDSYTSYSPSFQ | | | | | | | |
| | | GHVTISADKSISTAYLQ | | | | | | | |
| | | WSSLKASDTAMYYCAR | | | | | | | |
| | | LHYYNSEEFDVWGQGT | | | | | | | |
| | | MVTVSS | | | | | | | |
| mAb11- | VL | EIVLTQSPGTLSLSPGE | 879 | RASQSVSSS | 880 | GQSSRTR | 881 | QQFGNSQLFT | 882 |
| 1453 | | RATLSCRASQSVSSSYL | | YLA | | | | | |
| | | AWYQQKPGQAPRLLIY | | | | | | | |
| | | GQSSRTRGIPDRFSGS | | | | | | | |
| | | GSGTDFTLTISRLEPED | | | | | | | |
| | | FAVYYCQQFGNSQLFT | | | | | | | |
| | | FGQGTKLEIK | | | | | | | |
| mAb11- | VH | EVQLVQSGAEVKKPGE | 883 | TSWIS | 884 | MIDPSDSFTSYSPSFQ | 885 | LHYYHSEEFDV | 886 |
| 1454 | | SLRISCKGSGYSFTTSW | | | | G | | | |
| | | ISWVRQMPGKGLEWM | | | | | | | |
| | | GMIDPSDSFTSYSPSFQ | | | | | | | |
| | | GHVTISADKSISTAYLQ | | | | | | | |
| | | WSSLKASDTAMYYCAR | | | | | | | |
| | | LHYYHSEEFDVWGQGT | | | | | | | |
| | | MVTVSS | | | | | | | |
| mAb11- | VL | EIVLTQSPGTLSLSPGE | 887 | RASQSVSSS | 888 | GQSSRTR | 889 | QQFGNSQLFT | 890 |
| 1454 | | RATLSCRASQSVSSSYL | | YLA | | | | | |
| | | AWYQQKPGQAPRLLIY | | | | | | | |
| | | GQSSRTRGIPDRFSGS | | | | | | | |
| | | GSGTDFTLTISRLEPED | | | | | | | |
| | | FAVYYCQQFGNSQLFT | | | | | | | |
| | | FGQGTKLEIK | | | | | | | |
| mAb11- | VH | EVQLVQSGAEVKKPGE | 891 | TSWIS | 892 | MIDPSDSYTSYSPSFQ | 893 | LHYYHSEEFDV | 894 |
| 1455 | | SLRISCKGSGYSFTTSW | | | | G | | | |
| | | ISWVRQMPGKGLEWM | | | | | | | |
| | | GMIDPSDSYTSYSPSFQ | | | | | | | |
| | | GHVTISADKSISTAYLQ | | | | | | | |
| | | WSSLKASDTAMYYCAR | | | | | | | |
| | | LHYYHSEEFDVWGQGT | | | | | | | |
| | | LVTVSS | | | | | | | |
| mAb11- | VL | EIVLTQSPGTLSLSPGE | 895 | RASQSVSSS | 896 | GQSSRTR | 897 | QQFGNSQLFT | 898 |
| 1455 | | RATLSCRASQSVSSSYL | | YLA | | | | | |
| | | AWYQQKPGQAPRLLIY | | | | | | | |
| | | GQSSRTRGIPDRFSGS | | | | | | | |
| | | GSGTDFTLTISRLEPED | | | | | | | |
| | | FAVYYCQQFGNSQLFT | | | | | | | |
| | | FGQGTKLEIK | | | | | | | |
| mAb11- | VH | EVQLVQSGAEVKKPGE | 899 | TSWIS | 900 | MIDPSDSYTSYSPSFQ | 901 | LHYYHSEEFDV | 902 |
| 1456 | | SLRISCKGSGYSFTTSW | | | | G | | | |
| | | ISWVRQMPGKGLEWM | | | | | | | |
| | | GMIDPSDSYTSYSPSFQ | | | | | | | |
| | | GHVTISADKSISTAYLQ | | | | | | | |
| | | WSSLKASDTAMYYCAR | | | | | | | |
| | | LHYYHSEEFDVWGQGT | | | | | | | |
| | | MVTVSS | | | | | | | |
| mAb11- | VL | EIVLTQSPGTLSLSPGE | 903 | RASQSVSSS | 904 | GQSSRTR | 905 | QQFGNSQLFT | 906 |
| 1456 | | RATLSCRASQSVSSSYL | | YLA | | | | | |
| | | AWYQQKPGQAPRLLIY | | | | | | | |
| | | GQSSRTRGIPDRFSGS | | | | | | | |
| | | GSGTDFTLTISRLEPED | | | | | | | |
| | | FAVYYCQQFGNSQLFT | | | | | | | |
| | | FGQGTKLEIK | | | | | | | |
| mAb11- | VH | EVQLVQSGAEVKKPGE | 907 | TSWIS | 908 | MIDPSDSFTSYSPSFQ | 909 | LHYYNSEEFDV | 910 |
| 1457 | | SLRISCKGSGYSFSTSW | | | | G | | | |
| | | ISWVRQMPGKGLEWM | | | | | | | |
| | | GMIDPSDSFTSYSPSFQ | | | | | | | |
| | | GHVTISADKSISTAYLQ | | | | | | | |
| | | WSSLKASDTAMYYCAR | | | | | | | |
| | | LHYYNSEEFDVWGQGT | | | | | | | |
| | | LVTVSS | | | | | | | |
| mAb11- | VL | EIVLTQSPGTLSLSPGE | 911 | RASQSVSSS | 912 | GQSSRTR | 913 | QQFGNSQLFT | 914 |
| 1457 | | RATLSCRASQSVSSSYL | | YLA | | | | | |
| | | AWYQQKPGQAPRLLIY | | | | | | | |
| | | GQSSRTRGIPDRFSGS | | | | | | | |
| | | GSGTDFTLTISRLEPED | | | | | | | |
| | | FAVYYCQQFGNSQLFT | | | | | | | |
| | | FGQGTKLEIK | | | | | | | |
| mAb11- | VH | EVQLVQSGAEVKKPGE | 915 | TSWIV | 916 | MIDPSDSYTSYSPSFQ | 917 | LHYYNSEEFDV | 918 |
| 1458 | | SLRISCKGSGYSFSTSW | | | | G | | | |
| | | IVWVRQMPGKGLEWM | | | | | | | |
| | | GMIDPSDSYTSYSPSFQ | | | | | | | |
| | | GHVTISADKSISTAYLQ | | | | | | | |
| | | WSSLKASDTAMYYCAR | | | | | | | |
| | | LHYYNSEEFDVWGQGT | | | | | | | |
| | | LVTVSS | | | | | | | |
| mAb11- | VL | EIVLTQSPGTLSLSPGE | 919 | RASQSVSSS | 920 | GQSSRTR | 921 | QQFGNSQLFT | 922 |
| 1458 | | RATLSCRASQSVSSSYL | | YLA | | | | | |
| | | AWYQQKPGQAPRLLIY | | | | | | | |
| | | GQSSRTRGIPDRFSGS | | | | | | | |
| | | GSGTDFTLTISRLEPED | | | | | | | |
| | | FAVYYCQQFGNSQLFT | | | | | | | |
| | | FGQGTKLEIK | | | | | | | |
| mAb11- | VH | EVQLVQSGAEVKKPGE | 923 | TSWIV | 924 | MIDPSDSYTSYSPSFQ | 925 | LHYYNSEEFDV | 926 |
| 1459 | | SLRISCKGSGYSFSTSW | | | | G | | | |
| | | IVWVRQMPGKGLEWM | | | | | | | |
| | | GMIDPSDSYTSYSPSFQ | | | | | | | |
| | | GHVTISADKSISTAYLQ | | | | | | | |
| | | WSSLKASDTAMYYCAR | | | | | | | |
| | | LHYYNSEEFDVWGQGT | | | | | | | |
| | | MVTVSS | | | | | | | |
| mAb11- | VL | EIVLTQSPGTLSLSPGE | 927 | RASQSVSSS | 928 | GQSSRTR | 929 | QQFGNSQLFT | 930 |
| 1459 | | RATLSCRASQSVSSSYL | | YLA | | | | | |
| | | AWYQQKPGQAPRLLIY | | | | | | | |
| | | GQSSRTRGIPDRFSGS | | | | | | | |
| | | GSGTDFTLTISRLEPED | | | | | | | |
| | | FAVYYCQQFGNSQLFT | | | | | | | |
| | | FGQGTKLEIK | | | | | | | |
| mAb11- | VH | EVQLVQSGAEVKKPGE | 931 | TSWIS | 932 | MIDPSDSYTSYSPSFQ | 933 | LHYYNSEEFDV | 934 |
| 1460 | | SLRISCKGSGYSFSTSW | | | | G | | | |
| | | ISWVRQMPGKGLEWM | | | | | | | |
| | | GMIDPSDSYTSYSPSFQ | | | | | | | |
| | | GHVTISADKSISTAYLQ | | | | | | | |
| | | WSSLKASDTAMYYCAR | | | | | | | |
| | | LHYYNSEEFDVWGQGT | | | | | | | |
| | | MVTVSS | | | | | | | |
| mAb11- | VL | EIVLTQSPGTLSLSPGE | 935 | RASQSVSSS | 936 | GQSSRTR | 937 | QQFGQSQLFT | 938 |
| 1460 | | RATLSCRASQSVSSSYL | | YLA | | | | | |
| | | AWYQQKPGQAPRLLIY | | | | | | | |
| | | GQSSRTRGIPDRFSGS | | | | | | | |
| | | GSGTDFTLTISRLEPED | | | | | | | |
| | | FAVYYCQQFGQSQLFT | | | | | | | |
| | | FGQGTKLEIK | | | | | | | |
| mAb11- | VH | EVQLVQSGAEVKKPGE | 939 | TSWIS | 940 | MIDPSDSFTSYSPSFQ | 941 | LHYYHSEEFDV | 942 |
| 1461 | | SLRISCKGSGYSFTTSW | | | | G | | | |
| | | ISWVRQMPGKGLEWM | | | | | | | |
| | | GMIDPSDSFTSYSPSFQ | | | | | | | |
| | | GHVTISADKSISTAYLQ | | | | | | | |
| | | WSSLKASDTAMYYCAR | | | | | | | |
| | | LHYYHSEEFDVWGQGT | | | | | | | |
| | | MVTVSS | | | | | | | |
| mAb11- | VL | EIVLTQSPGTLSLSPGE | 943 | RASQSVSSS | 944 | GQSSRTR | 945 | QQFGQSQLFT | 946 |
| 1461 | | RATLSCRASQSVSSSYL | | YLA | | | | | |
| | | AWYQQKPGQAPRLLIY | | | | | | | |
| | | GQSSRTRGIPDRFSGS | | | | | | | |
| | | GSGTDFTLTISRLEPED | | | | | | | |
| | | FAVYYCQQFGQSQLFT | | | | | | | |
| | | FGQGTKLEIK | | | | | | | |
| mAb11- | VH | EVQLVQSGAEVKKPGE | 947 | TSWIS | 948 | MIDPSDSYTSYSPSFQ | 949 | LHYYHSEEFDV | 950 |
| 1463 | | SLRISCKGSGYSFTTSW | | | | G | | | |
| | | ISWVRQMPGKGLEWM | | | | | | | |
| | | GMIDPSDSYTSYSPSFQ | | | | | | | |
| | | GHVTISADKSISTAYLQ | | | | | | | |
| | | WSSLKASDTAMYYCAR | | | | | | | |
| | | LHYYHSEEFDVWGQGT | | | | | | | |
| | | MVTVSS | | | | | | | |
| mAb11- | VL | EIVLTQSPGTLSLSPGE | 951 | RASQSVSSS | 952 | GQSSRTR | 953 | QQFGQSQLFT | 954 |
| 1463 | | RATLSCRASQSVSSSYL | | YLA | | | | | |
| | | AWYQQKPGQAPRLLIY | | | | | | | |
| | | GQSSRTRGIPDRFSGS | | | | | | | |
| | | GSGTDFTLTISRLEPED | | | | | | | |
| | | FAVYYCQQFGQSQLFT | | | | | | | |
| | | FGQGTKLEIK | | | | | | | |
| mAb11- | VH | EVQLVQSGAEVKKPGE | 955 | TSWIS | 956 | MIDPSDSFTSYSPSFQ | 957 | LHYYNSEEFDV | 958 |
| 1464 | | SLRISCKGSGYSFSTSW | | | | G | | | |
| | | ISWVRQMPGKGLEWM | | | | | | | |
| | | GMIDPSDSFTSYSPSFQ | | | | | | | |
| | | GHVTISADKSISTAYLQ | | | | | | | |
| | | WSSLKASDTAMYYCAR | | | | | | | |
| | | LHYYNSEEFDVWGQGT | | | | | | | |
| | | LVTVSS | | | | | | | |
| mAb11- | VL | EIVLTQSPGTLSLSPGE | 959 | RASQSVSSS | 960 | GQSSRTR | 961 | QQFGQSQLFT | 962 |
| 1464 | | RATLSCRASQSVSSSYL | | YLA | | | | | |
| | | AWYQQKPGQAPRLLIY | | | | | | | |
| | | GQSSRTRGIPDRFSGS | | | | | | | |
| | | GSGTDFTLTISRLEPED | | | | | | | |
| | | FAVYYCQQFGQSQLFT | | | | | | | |
| | | FGQGTKLEIK | | | | | | | |
| mAb11- | VH | EVQLVQSGAEVKKPGE | 963 | TSWIV | 964 | MIDPSDSYTSYSPSFQ | 965 | LHYYNSEEFDV | 966 |
| 1465 | | SLRISCKGSGYSFSTSW | | | | G | | | |
| | | IVWVRQMPGKGLEWM | | | | | | | |
| | | GMIDPSDSYTSYSPSFQ | | | | | | | |
| | | GHVTISADKSISTAYLQ | | | | | | | |
| | | WSSLKASDTAMYYCAR | | | | | | | |
| | | LHYYNSEEFDVWGQGT | | | | | | | |
| | | LVTVSS | | | | | | | |
| mAb11- | VL | EIVLTQSPGTLSLSPGE | 967 | RASQSVSSS | 968 | GQSSRTR | 969 | QQFGQSQLFT | 970 |
| 1465 | | RATLSCRASQSVSSSYL | | YLA | | | | | |
| | | AWYQQKPGQAPRLLIY | | | | | | | |
| | | GQSSRTRGIPDRFSGS | | | | | | | |
| | | GSGTDFTLTISRLEPED | | | | | | | |
| | | FAVYYCQQFGQSQLFT | | | | | | | |
| | | FGQGTKLEIK | | | | | | | |
| mAb11- | VH | EVQLVQSGAEVKKPGE | 971 | TSWIV | 972 | MIDPSDSYTSYSPSFQ | 973 | LHYYNSEEFDV | 974 |
| 1466 | | SLRISCKGSGYSFSTSW | | | | G | | | |
| | | IVWVRQMPGKGLEWM | | | | | | | |
| | | GMIDPSDSYTSYSPSFQ | | | | | | | |
| | | GHVTISADKSISTAYLQ | | | | | | | |
| | | WSSLKASDTAMYYCAR | | | | | | | |
| | | LHYYNSEEFDVWGQGT | | | | | | | |
| | | MVTVSS | | | | | | | |
| mAb11- | VL | EIVLTQSPGTLSLSPGE | 975 | RASQSVSSS | 976 | GQSSRTR | 977 | QQFGQSQLFT | 978 |
| 1466 | | RATLSCRASQSVSSSYL | | YLA | | | | | |
| | | AWYQQKPGQAPRLLIY | | | | | | | |
| | | GQSSRTRGIPDRFSGS | | | | | | | |
| | | GSGTDFTLTISRLEPED | | | | | | | |
| | | FAVYYCQQFGQSQLFT | | | | | | | |
| | | FGQGTKLEIK | | | | | | | |
| mAb11- | VH | EVQLVQSGAEVKKPGE | 979 | TSWIS | 980 | MIDPSDSYTSYSPSFQ | 981 | LHYYNSEEFDV | 982 |
| 1467 | | SLRISCKGSGYSFSTSW | | | | G | | | |
| | | ISWVRQMPGKGLEWM | | | | | | | |
| | | GMIDPSDSYTSYSPSFQ | | | | | | | |
| | | GHVTISADKSISTAYLQ | | | | | | | |
| | | WSSLKASDTAMYYCAR | | | | | | | |
| | | LHYYNSEEFDVWGQGT | | | | | | | |
| | | MVTVSS | | | | | | | |
| mAb11- | VL | EIVLTQSPGTLSLSPGE | 983 | RASQSVSSS | 984 | GQSSRTR | 985 | QQFGDAQLFT | 986 |
| 1467 | | RATLSCRASQSVSSSYL | | YLA | | | | | |
| | | AWYQQKPGQAPRLLIY | | | | | | | |
| | | GQSSRTRGIPDRFSGS | | | | | | | |
| | | GSGTDFTLTISRLEPED | | | | | | | |
| | | FAVYYCQQFGDAQLFT | | | | | | | |
| | | FGQGTKLEIK | | | | | | | |
| mAb11- | VH | EVQLVQSGAEVKKPGE | 987 | TSWIS | 988 | MIDPSDSFTSYSPSFQ | 989 | LHYYHSEEFDV | 990 |
| 1468 | | SLRISCKGSGYSFTTSW | | | | G | | | |
| | | ISWVRQMPGKGLEWM | | | | | | | |
| | | GMIDPSDSFTSYSPSFQ | | | | | | | |
| | | GHVTISADKSISTAYLQ | | | | | | | |
| | | WSSLKASDTAMYYCAR | | | | | | | |
| | | LHYYHSEEFDVWGQGT | | | | | | | |
| | | MVTVSS | | | | | | | |
| mAb11- | VL | EIVLTQSPGTLSLSPGE | 991 | RASQSVSSS | 992 | GQSSRTR | 993 | QQFGDAQLFT | 994 |
| 1468 | | RATLSCRASQSVSSSYL | | YLA | | | | | |
| | | AWYQQKPGQAPRLLIY | | | | | | | |
| | | GQSSRTRGIPDRFSGS | | | | | | | |
| | | GSGTDFTLTISRLEPED | | | | | | | |
| | | FAVYYCQQFGDAQLFT | | | | | | | |
| | | FGQGTKLEIK | | | | | | | |
| mAb11- | VH | EVQLVQSGAEVKKPGE | 995 | TSWIS | 996 | MIDPSDSYTSYSPSFQ | 997 | LHYYHSEEFDV | 998 |
| 1470 | | SLRISCKGSGYSFTTSW | | | | G | | | |
| | | ISWVRQMPGKGLEWM | | | | | | | |
| | | GMIDPSDSYTSYSPSFQ | | | | | | | |
| | | GHVTISADKSISTAYLQ | | | | | | | |
| | | WSSLKASDTAMYYCAR | | | | | | | |
| | | LHYYHSEEFDVWGQGT | | | | | | | |
| | | MVTVSS | | | | | | | |
| mAb11- | VL | EIVLTQSPGTLSLSPGE | 999 | RASQSVSSS | 1000 | GQSSRTR | 1001 | QQFGDAQLFT | 1002 |
| 1470 | | RATLSCRASQSVSSSYL | | YLA | | | | | |
| | | AWYQQKPGQAPRLLIY | | | | | | | |
| | | GQSSRTRGIPDRFSGS | | | | | | | |
| | | GSGTDFTLTISRLEPED | | | | | | | |
| | | FAVYYCQQFGDAQLFT | | | | | | | |
| | | FGQGTKLEIK | | | | | | | |
| mAb11- | VH | EVQLVQSGAEVKKPGE | 1003 | TSWIS | 1004 | MIDPSDSFTSYSPSFQ | 1005 | LHYYNSEEFDV | 1006 |
| 1471 | | SLRISCKGSGYSFSTSW | | | | G | | | |
| | | ISWVRQMPGKGLEWM | | | | | | | |
| | | GMIDPSDSFTSYSPSFQ | | | | | | | |
| | | GHVTISADKSISTAYLQ | | | | | | | |
| | | WSSLKASDTAMYYCAR | | | | | | | |
| | | LHYYNSEEFDVWGQGT | | | | | | | |
| | | LVTVSS | | | | | | | |
| mAb11- | VL | EIVLTQSPGTLSLSPGE | 1007 | RASQSVSSS | 1008 | GQSSRTR | 1009 | QQFGDAQLFT | 1010 |
| 1471 | | RATLSCRASQSVSSSYL | | YLA | | | | | |
| | | AWYQQKPGQAPRLLIY | | | | | | | |
| | | GQSSRTRGIPDRFSGS | | | | | | | |
| | | GSGTDFTLTISRLEPED | | | | | | | |
| | | FAVYYCQQFGDAQLFT | | | | | | | |
| | | FGQGTKLEIK | | | | | | | |
| mAb11- | VH | EVQLVQSGAEVKKPGE | 1011 | TSWIV | 1012 | MIDPSDSYTSYSPSFQ | 1013 | LHYYNSEEFDV | 1014 |
| 1472 | | SLRISCKGSGYSFSTSW | | | | G | | | |
| | | IVWVRQMPGKGLEWM | | | | | | | |
| | | GMIDPSDSYTSYSPSFQ | | | | | | | |
| | | GHVTISADKSISTAYLQ | | | | | | | |
| | | WSSLKASDTAMYYCAR | | | | | | | |
| | | LHYYNSEEFDVWGQGT | | | | | | | |
| | | LVTVSS | | | | | | | |
| mAb11- | VL | EIVLTQSPGTLSLSPGE | 1015 | RASQSVSSS | 1016 | GQSSRTR | 1017 | QQFGDAQLFT | 1018 |
| 1472 | | RATLSCRASQSVSSSYL | | YLA | | | | | |
| | | AWYQQKPGQAPRLLIY | | | | | | | |
| | | GQSSRTRGIPDRFSGS | | | | | | | |
| | | GSGTDFTLTISRLEPED | | | | | | | |
| | | FAVYYCQQFGDAQLFT | | | | | | | |
| | | FGQGTKLEIK | | | | | | | |
| mAb11- | VH | EVQLVQSGAEVKKPGE | 1019 | TSWIV | 1020 | MIDPSDSYTSYSPSFQ | 1021 | LHYYNSEEFDV | 1022 |
| 1473 | | SLRISCKGSGYSFSTSW | | | | G | | | |
| | | IVWVRQMPGKGLEWM | | | | | | | |
| | | GMIDPSDSYTSYSPSFQ | | | | | | | |
| | | GHVTISADKSISTAYLQ | | | | | | | |
| | | WSSLKASDTAMYYCAR | | | | | | | |
| | | LHYYNSEEFDVWGQGT | | | | | | | |
| | | MVTVSS | | | | | | | |
| mAb11- | VL | EIVLTQSPGTLSLSPGE | 1023 | RASQSVSSS | 1024 | GQSSRTR | 1025 | QQFGDAQLFT | 1026 |
| 1473 | | RATLSCRASQSVSSSYL | | YLA | | | | | |
| | | AWYQQKPGQAPRLLIY | | | | | | | |
| | | GQSSRTRGIPDRFSGS | | | | | | | |
| | | GSGTDFTLTISRLEPED | | | | | | | |
| | | FAVYYCQQFGDAQLFT | | | | | | | |
| | | FGQGTKLEIK | | | | | | | |
| mAb11- | VH | EVQLVQSGAEVKKPGE | 1027 | TSWIS | 1028 | MIDPSDSYTSYSPSFQ | 1029 | LHYYNSEEFDV | 1030 |
| 1474 | | SLRISCKGSGYSFSTSW | | | | G | | | |
| | | ISWVRQMPGKGLEWM | | | | | | | |
| | | GMIDPSDSYTSYSPSFQ | | | | | | | |
| | | GHVTISADKSISTAYLQ | | | | | | | |
| | | WSSLKASDTAMYYCAR | | | | | | | |
| | | LHYYNSEEFDVWGQGT | | | | | | | |
| | | MVTVSS | | | | | | | |
| mAb11- | VL | EIVLTQSPGTLSLSPGE | 1031 | RASQSVSSS | 1032 | GQSSRTR | 1033 | QQFGDTQLFT | 1034 |
| 1474 | | RATLSCRASQSVSSSYL | | YLA | | | | | |
| | | AWYQQKPGQAPRLLIY | | | | | | | |
| | | GQSSRTRGIPDRFSGS | | | | | | | |
| | | GSGTDFTLTISRLEPED | | | | | | | |
| | | FAVYYCQQFGDTQLFT | | | | | | | |
| | | FGQGTKLEIK | | | | | | | |
| mAb11- | VH | EVQLVQSGAEVKKPGE | 1035 | TSWIS | 1036 | MIDPSDSFTSYSPSFQ | 1037 | LHYYHSEEFDV | 1038 |
| 1475 | | SLRISCKGSGYSFTTSW | | | | G | | | |
| | | ISWVRQMPGKGLEWM | | | | | | | |
| | | GMIDPSDSFTSYSPSFQ | | | | | | | |
| | | GHVTISADKSISTAYLQ | | | | | | | |
| | | WSSLKASDTAMYYCAR | | | | | | | |
| | | LHYYHSEEFDVWGQGT | | | | | | | |
| | | MVTVSS | | | | | | | |
| mAb11- | VL | EIVLTQSPGTLSLSPGE | 1039 | RASQSVSSS | 1040 | GQSSRTR | 1041 | QQFGDTQLFT | 1042 |
| 1475 | | RATLSCRASQSVSSSYL | | YLA | | | | | |
| | | AWYQQKPGQAPRLLIY | | | | | | | |
| | | GQSSRTRGIPDRFSGS | | | | | | | |
| | | GSGTDFTLTISRLEPED | | | | | | | |
| | | FAVYYCQQFGDTQLFT | | | | | | | |
| | | FGQGTKLEIK | | | | | | | |
| mAb11- | VH | EVQLVQSGAEVKKPGE | 1043 | TSWIS | 1044 | MIDPSDSYTSYSPSFQ | 1045 | LHYYHSEEFDV | 1046 |
| 1476 | | SLRISCKGSGYSFTTSW | | | | G | | | |
| | | ISWVRQMPGKGLEWM | | | | | | | |
| | | GMIDPSDSYTSYSPSFQ | | | | | | | |
| | | GHVTISADKSISTAYLQ | | | | | | | |
| | | WSSLKASDTAMYYCAR | | | | | | | |
| | | LHYYHSEEFDVWGQGT | | | | | | | |
| | | LVTVSS | | | | | | | |
| mAb11- | VL | EIVLTQSPGTLSLSPGE | 1047 | RASQSVSSS | 1048 | GQSSRTR | 1049 | QQFGDTQLFT | 1050 |
| 1476 | | RATLSCRASQSVSSSYL | | YLA | | | | | |
| | | AWYQQKPGQAPRLLIY | | | | | | | |
| | | GQSSRTRGIPDRFSGS | | | | | | | |
| | | GSGTDFTLTISRLEPED | | | | | | | |
| | | FAVYYCQQFGDTQLFT | | | | | | | |
| | | FGQGTKLEIK | | | | | | | |
| mAb11- | VH | EVQLVQSGAEVKKPGE | 1051 | TSWIS | 1052 | MIDPSDSYTSYSPSFQ | 1053 | LHYYHSEEFDV | 1054 |
| 1477 | | SLRISCKGSGYSFTTSW | | | | G | | | |
| | | ISWVRQMPGKGLEWM | | | | | | | |
| | | GMIDPSDSYTSYSPSFQ | | | | | | | |
| | | GHVTISADKSISTAYLQ | | | | | | | |
| | | WSSLKASDTAMYYCAR | | | | | | | |
| | | LHYYHSEEFDVWGQGT | | | | | | | |
| | | MVTVSS | | | | | | | |
| mAb11- | VL | EIVLTQSPGTLSLSPGE | 1055 | RASQSVSSS | 1056 | GQSSRTR | 1057 | QQFGDTQLFT | 1058 |
| 1477 | | RATLSCRASQSVSSSYL | | YLA | | | | | |
| | | AWYQQKPGQAPRLLIY | | | | | | | |
| | | GQSSRTRGIPDRFSGS | | | | | | | |
| | | GSGTDFTLTISRLEPED | | | | | | | |
| | | FAVYYCQQFGDTQLFT | | | | | | | |
| | | FGQGTKLEIK | | | | | | | |
| mAb11- | VH | EVQLVQSGAEVKKPGE | 1059 | TSWIS | 1060 | MIDPSDSFTSYSPSFQ | 1061 | LHYYNSEEFDV | 1062 |
| 1478 | | SLRISCKGSGYSFSTSW | | | | G | | | |
| | | ISWVRQMPGKGLEWM | | | | | | | |
| | | GMIDPSDSFTSYSPSFQ | | | | | | | |
| | | GHVTISADKSISTAYLQ | | | | | | | |
| | | WSSLKASDTAMYYCAR | | | | | | | |
| | | LHYYNSEEFDVWGQGT | | | | | | | |
| | | LVTVSS | | | | | | | |
| mAb11- | VL | EIVLTQSPGTLSLSPGE | 1063 | RASQSVSSS | 1064 | GQSSRTR | 1065 | QQFGDTQLFT | 1066 |
| 1478 | | RATLSCRASQSVSSSYL | | YLA | | | | | |
| | | AWYQQKPGQAPRLLIY | | | | | | | |
| | | GQSSRTRGIPDRFSGS | | | | | | | |
| | | GSGTDFTLTISRLEPED | | | | | | | |
| | | FAVYYCQQFGDTQLFT | | | | | | | |
| | | FGQGTKLEIK | | | | | | | |
| mAb11- | VH | EVQLVQSGAEVKKPGE | 1067 | TSWIV | 1068 | MIDPSDSYTSYSPSFQ | 1069 | LHYYNSEEFDV | 1070 |
| 1479 | | SLRISCKGSGYSFSTSW | | | | G | | | |
| | | IVWVRQMPGKGLEWM | | | | | | | |
| | | GMIDPSDSYTSYSPSFQ | | | | | | | |
| | | GHVTISADKSISTAYLQ | | | | | | | |
| | | WSSLKASDTAMYYCAR | | | | | | | |
| | | LHYYNSEEFDVWGQGT | | | | | | | |
| | | LVTVSS | | | | | | | |
| mAb11- | VL | EIVLTQSPGTLSLSPGE | 1071 | RASQSVSSS | 1072 | GQSSRTR | 1073 | QQFGDTQLFT | 1074 |
| 1479 | | RATLSCRASQSVSSSYL | | YLA | | | | | |
| | | AWYQQKPGQAPRLLIY | | | | | | | |
| | | GQSSRTRGIPDRFSGS | | | | | | | |
| | | GSGTDFTLTISRLEPED | | | | | | | |
| | | FAVYYCQQFGDTQLFT | | | | | | | |
| | | FGQGTKLEIK | | | | | | | |
| mAb11- | VH | EVQLVQSGAEVKKPGE | 1075 | TSWIV | 1076 | MIDPSDSYTSYSPSFQ | 1077 | LHYYNSEEFDV | 1078 |
| 1480 | | SLRISCKGSGYSFSTSW | | | | G | | | |
| | | IVWVRQMPGKGLEWM | | | | | | | |
| | | GMIDPSDSYTSYSPSFQ | | | | | | | |
| | | GHVTISADKSISTAYLQ | | | | | | | |
| | | WSSLKASDTAMYYCAR | | | | | | | |
| | | LHYYNSEEFDVWGQGT | | | | | | | |
| | | MVTVSS | | | | | | | |
| mAb11- | VL | EIVLTQSPGTLSLSPGE | 1079 | RASQSVSSS | 1080 | GQSSRTR | 1081 | QQFGDTQLFT | 1082 |
| 1480 | | RATLSCRASQSVSSSYL | | YLA | | | | | |
| | | AWYQQKPGQAPRLLIY | | | | | | | |
| | | GQSSRTRGIPDRFSGS | | | | | | | |
| | | GSGTDFTLTISRLEPED | | | | | | | |
| | | FAVYYCQQFGDTQLFT | | | | | | | |
| | | FGQGTKLEIK | | | | | | | |
| mAb11- | VH | EVQLVQSGAEVKKPGE | 1083 | TSWIS | 1084 | MIDPSDSYTSYSPSFQ | 1085 | LHYYNSEEFDV | 1086 |
| 1481 | | SLRISCKGSGYSFSTSW | | | | G | | | |
| | | ISWVRQMPGKGLEWM | | | | | | | |
| | | GMIDPSDSYTSYSPSFQ | | | | | | | |
| | | GHVTISADKSISTAYLQ | | | | | | | |
| | | WSSLKASDTAMYYCAR | | | | | | | |
| | | LHYYNSEEFDVWGQGT | | | | | | | |
| | | MVTVSS | | | | | | | |
| mAb11- | VL | EIVLTQSPGTLSLSPGE | 1087 | RASQSVSSS | 1088 | GQSSRTR | 1089 | QQFGDNQLFT | 1090 |
| 1481 | | RATLSCRASQSVSSSYL | | YLA | | | | | |
| | | AWYQQKPGQAPRLLIY | | | | | | | |
| | | GQSSRTRGIPDRFSGS | | | | | | | |
| | | GSGTDFTLTISRLEPED | | | | | | | |
| | | FAVYYCQQFGDNQLFT | | | | | | | |
| | | FGQGTKLEIK | | | | | | | |
| mAb11- | VH | EVQLVQSGAEVKKPGE | 1091 | TSWIS | 1092 | MIDPSDSFTSYSPSFQ | 1093 | LHYYHSEEFDV | 1094 |
| 1482 | | SLRISCKGSGYSFTTSW | | | | G | | | |
| | | ISWVRQMPGKGLEWM | | | | | | | |
| | | GMIDPSDSFTSYSPSFQ | | | | | | | |
| | | GHVTISADKSISTAYLQ | | | | | | | |
| | | WSSLKASDTAMYYCAR | | | | | | | |
| | | LHYYHSEEFDVWGQGT | | | | | | | |
| | | MVTVSS | | | | | | | |
| mAb11- | VL | EIVLTQSPGTLSLSPGE | 1095 | RASQSVSSS | 1096 | GQSSRTR | 1097 | QQFGDNQLFT | 1098 |
| 1482 | | RATLSCRASQSVSSSYL | | YLA | | | | | |
| | | AWYQQKPGQAPRLLIY | | | | | | | |
| | | GQSSRTRGIPDRFSGS | | | | | | | |
| | | GSGTDFTLTISRLEPED | | | | | | | |
| | | FAVYYCQQFGDNQLFT | | | | | | | |
| | | FGQGTKLEIK | | | | | | | |
| mAb11- | VH | EVQLVQSGAEVKKPGE | 1099 | TSWIS | 1100 | MIDPSDSYTSYSPSFQ | 1101 | LHYYHSEEFDV | 1102 |
| 1483 | | SLRISCKGSGYSFTTSW | | | | G | | | |
| | | ISWVRQMPGKGLEWM | | | | | | | |
| | | GMIDPSDSYTSYSPSFQ | | | | | | | |
| | | GHVTISADKSISTAYLQ | | | | | | | |
| | | WSSLKASDTAMYYCAR | | | | | | | |
| | | LHYYHSEEFDVWGQGT | | | | | | | |
| | | LVTVSS | | | | | | | |
| mAb11- | VL | EIVLTQSPGTLSLSPGE | 1103 | RASQSVSSS | 1104 | GQSSRTR | 1105 | QQFGDNQLFT | 1106 |
| 1483 | | RATLSCRASQSVSSSYL | | YLA | | | | | |
| | | AWYQQKPGQAPRLLIY | | | | | | | |
| | | GQSSRTRGIPDRFSGS | | | | | | | |
| | | GSGTDFTLTISRLEPED | | | | | | | |
| | | FAVYYCQQFGDNQLFT | | | | | | | |
| | | FGQGTKLEIK | | | | | | | |
| mAb11- | VH | EVQLVQSGAEVKKPGE | 1107 | TSWIS | 1108 | MIDPSDSYTSYSPSFQ | 1109 | LHYYHSEEFDV | 1110 |
| 1484 | | SLISCKGSGYSFTTSW | | | | G | | | |
| | | ISWVRQMPGKGLEWM | | | | | | | |
| | | GMIDPSDSYTSYSPSFQ | | | | | | | |
| | | GHVTISADKSISTAYLQ | | | | | | | |
| | | WSSLKASDTAMYYCAR | | | | | | | |
| | | LHYYHSEEFDVWGQGT | | | | | | | |
| | | MVTVSS | | | | | | | |
| mAb11- | VL | EIVLTQSPGTLSLSPGE | 1111 | RASQSVSSS | 1112 | GQSSRTR | 1113 | QQFGDNQLFT | 1114 |
| 1484 | | RATLSCRASQSVSSSYL | | YLA | | | | | |
| | | AWYQQKPGQAPRLLIY | | | | | | | |
| | | GQSSRTRGIPDRFSGS | | | | | | | |
| | | GSGTDFTLTISRLEPED | | | | | | | |
| | | FAVYYCQQFGDNQLFT | | | | | | | |
| | | FGQGTKLEIK | | | | | | | |
| mAb11- | VH | EVQLVQSGAEVKKPGE | 1115 | TSWIS | 1116 | MIDPSDSFTSYSPSFQ | 1117 | LHYYNSEEFDV | 1118 |
| 1485 | | SLRISCKGSGYSFSTSW | | | | G | | | |
| | | ISWVRQMPGKGLEWM | | | | | | | |
| | | GMIDPSDSFTSYSPSFQ | | | | | | | |
| | | GHVTISADKSISTAYLQ | | | | | | | |
| | | WSSLKASDTAMYYCAR | | | | | | | |
| | | LHYYNSEEFDVWGQGT | | | | | | | |
| | | LVTVSS | | | | | | | |
| mAb11- | VL | EIVLTQSPGTLSLSPGE | 1119 | RASQSVSSS | 1120 | GQSSRTR | 1121 | QQFGDNQLFT | 1122 |
| 1485 | | RATLSCRASQSVSSSYL | | YLA | | | | | |
| | | AWYQQKPGQAPRLLIY | | | | | | | |
| | | GQSSRTRGIPDRFSGS | | | | | | | |
| | | GSGTDFTLTISRLEPED | | | | | | | |
| | | FAVYYCQQFGDNQLFT | | | | | | | |
| | | FGQGTKLEIK | | | | | | | |
| mAb11- | VH | EVQLVQSGAEVKKPGE | 1123 | TSWIV | 1124 | MIDPSDSYTSYSPSFQ | 1125 | LHYYNSEEFDV | 1126 |
| 1486 | | SLRISCKGSGYSFSTSW | | | | G | | | |
| | | IVWVRQMPGKGLEWM | | | | | | | |
| | | GMIDPSDSYTSYSPSFQ | | | | | | | |
| | | GHVTISADKSISTAYLQ | | | | | | | |
| | | WSSLKASDTAMYYCAR | | | | | | | |
| | | LHYYNSEEFDVWGQGT | | | | | | | |
| | | LVTVSS | | | | | | | |
| mAb11- | VL | EIVLTQSPGTLSLSPGE | 1127 | RASQSVSSS | 1128 | GQSSRTR | 1129 | QQFGDNQLFT | 1130 |
| 1486 | | RATLSCRASQSVSSSYL | | YLA | | | | | |
| | | AWYQQKPGQAPRLLIY | | | | | | | |
| | | GQSSRTRGIPDRFSGS | | | | | | | |
| | | GSGTDFTLTISRLEPED | | | | | | | |
| | | FAVYYCQQFGDNQLFT | | | | | | | |
| | | FGQGTKLEIK | | | | | | | |
| mAb11- | VH | EVQLVQSGAEVKKPGE | 1131 | TSWIV | 1132 | MIDPSDSYTSYSPSFQ | 1133 | LHYYNSEEFDV | 1134 |
| 1487 | | SLRISCKGSGYSFSTSW | | | | G | | | |
| | | IVWVRQMPGKGLEWM | | | | | | | |
| | | GMIDPSDSYTSYSPSFQ | | | | | | | |
| | | GHVTISADKSISTAYLQ | | | | | | | |
| | | WSSLKASDTAMYYCAR | | | | | | | |
| | | LHYYNSEEFDVWGQGT | | | | | | | |
| | | MVTVSS | | | | | | | |
| mAb11- | VL | EIVLTQSPGTLSLSPGE | 1135 | RASQSVSSS | 1136 | GQSSRTR | 1137 | QQFGDNQLFT | 1138 |
| 1487 | | RATLSCRASQSVSSSYL | | YLA | | | | | |
| | | AWYQQKPGQAPRLLIY | | | | | | | |
| | | GQSSRTRGIPDRFSGS | | | | | | | |
| | | GSGTDFTLTISRLEPED | | | | | | | |
| | | FAVYYCQQFGDNQLFT | | | | | | | |
| | | FGQGTKLEIK | | | | | | | |
| mAb11- | VH | EVQLVQSGAEVKKPGE | 1139 | TSWIS | 1140 | MIDPSDSYTSYSPSFQ | 1141 | LHYYNSEEFDV | 1142 |
| 1488 | | SLRISCKGSGYSFSTSW | | | | G | | | |
| | | ISWVRQMPGKGLEWM | | | | | | | |
| | | GMIDPSDSYTSYSPSFQ | | | | | | | |
| | | GHVTISADKSISTAYLQ | | | | | | | |
| | | WSSLKASDTAMYYCAR | | | | | | | |
| | | LHYYNSEEFDVWGQGT | | | | | | | |
| | | MVTVSS | | | | | | | |
| mAb11- | VL | EIVLTQSPGTLSLSPGE | 1143 | RASQSVSSS | 1144 | GQSSRTR | 1145 | QQFGDDQLFT | 1146 |
| 1488 | | RATLSCRASQSVSSSYL | | YLA | | | | | |
| | | AWYQQKPGQAPRLLIY | | | | | | | |
| | | GQSSRTRGIPDRFSGS | | | | | | | |
| | | GSGTDFTLTISRLEPED | | | | | | | |
| | | FAVYYCQQFGDDQLFT | | | | | | | |
| | | FGQGTKLEIK | | | | | | | |
| mAb11- | VH | EVQLVQSGAEVKKPGE | 1147 | TSWIS | 1148 | MIDPSDSFTSYSPSFQ | 1149 | LHYYHSEEFDV | 1150 |
| 1489 | | SLRISCKGSGYSFTTSW | | | | G | | | |
| | | ISWVRQMPGKGLEWM | | | | | | | |
| | | GMIDPSDSFTSYSPSFQ | | | | | | | |
| | | GHVTISADKSISTAYLQ | | | | | | | |
| | | WSSLKASDTAMYYCAR | | | | | | | |
| | | LHYYHSEEFDVWGQGT | | | | | | | |
| | | MVTVSS | | | | | | | |
| mAb11- | VL | EIVLTQSPGTLSLSPGE | 1151 | RASQSVSSS | 1152 | GQSSRTR | 1153 | QQFGDDQLFT | 1154 |
| 1489 | | RATLSCRASQSVSSSYL | | YLA | | | | | |
| | | AWYQQKPGQAPRLLIY | | | | | | | |
| | | GQSSRTRGIPDRFSGS | | | | | | | |
| | | GSGTDFTLTISRLEPED | | | | | | | |
| | | FAVYYCQQFGDDQLFT | | | | | | | |
| | | FGQGTKLEIK | | | | | | | |
| mAb11- | VH | EVQLVQSGAEVKKPGE | 1155 | TSWIS | 1156 | MIDPSDSYTSYSPSFQ | 1157 | LHYYHSEEFDV | 1158 |
| 1490 | | SLRISCKGSGYSFTTSW | | | | G | | | |
| | | ISWVRQMPGKGLEWM | | | | | | | |
| | | GMIDPSDSYTSYSPSFQ | | | | | | | |
| | | GHVTISADKSISTAYLQ | | | | | | | |
| | | WSSLKASDTAMYYCAR | | | | | | | |
| | | LHYYHSEEFDVWGQGT | | | | | | | |
| | | LVTVSS | | | | | | | |
| mAb11- | VL | EIVLTQSPGTLSLSPGE | 1159 | RASQSVSSS | 1160 | GQSSRTR | 1161 | QQFGDDQLFT | 1162 |
| 1490 | | RATLSCRASQSVSSSYL | | YLA | | | | | |
| | | AWYQQKPGQAPRLLIY | | | | | | | |
| | | GQSSRTRGIPDRFSGS | | | | | | | |
| | | GSGTDFTLTISRLEPED | | | | | | | |
| | | FAVYYCQQFGDDQLFT | | | | | | | |
| | | FGQGTKLEIK | | | | | | | |
| mAb11- | VH | EVQLVQSGAEVKKPGE | 1163 | TSWIS | 1164 | MIDPSDSYTSYSPSFQ | 1165 | LHYYHSEEFDV | 1166 |
| 1491 | | SLRISCKGSGYSFTTSW | | | | G | | | |
| | | ISWVRQMPGKGLEWM | | | | | | | |
| | | GMIDPSDSYTSYSPSFQ | | | | | | | |
| | | GHVTISADKSISTAYLQ | | | | | | | |
| | | WSSLKASDTAMYYCAR | | | | | | | |
| | | LHYYHSEEFDVWGQGT | | | | | | | |
| | | MVTVSS | | | | | | | |
| mAb11- | VL | EIVLTQSPGTLSLSPGE | 1167 | RASQSVSSS | 1168 | GQSSRTR | 1169 | QQFGDDQLFT | 1170 |
| 1491 | | RATLSCRASQSVSSSYL | | YLA | | | | | |
| | | AWYQQKPGQAPRLLIY | | | | | | | |
| | | GQSSRTRGIPDRFSGS | | | | | | | |
| | | GSGTDFTLTISRLEPED | | | | | | | |
| | | FAVYYCQQFGDDQLFT | | | | | | | |
| | | FGQGTKLEIK | | | | | | | |
| mAb11- | VH | EVQLVQSGAEVKKPGE | 1171 | TSWIS | 1172 | MIDPSDSFTSYSPSFQ | 1173 | LHYYNSEEFDV | 1174 |
| 1492 | | SLRISCKGSGYSFSTSW | | | | G | | | |
| | | ISWVRQMPGKGLEWM | | | | | | | |
| | | GMIDPSDSFTSYSPSFQ | | | | | | | |
| | | GHVTISADKSISTAYLQ | | | | | | | |
| | | WSSLKASDTAMYYCAR | | | | | | | |
| | | LHYYNSEEFDVWGQGT | | | | | | | |
| | | LVTVSS | | | | | | | |
| mAb11- | VL | EIVLTQSPGTLSLSPGE | 1175 | RASQSVSSS | 1176 | GQSSRTR | 1177 | QQFGDDQLFT | 1178 |
| 1492 | | RATLSCRASQSVSSSYL | | YLA | | | | | |
| | | AWYQQKPGQAPRLLIY | | | | | | | |
| | | GQSSRTRGIPDRFSGS | | | | | | | |
| | | GSGTDFTLTISRLEPED | | | | | | | |
| | | FAVYYCQQFGDDQLFT | | | | | | | |
| | | FGQGTKLEIK | | | | | | | |
| mAb11- | VH | EVQLVQSGAEVKKPGE | 1179 | TSWIV | 1180 | MIDPSDSYTSYSPSFQ | 1181 | LHYYNSEEFDV | 1182 |
| 1493 | | SLRISCKGSGYSFSTSW | | | | G | | | |
| | | IVWVRQMPGKGLEWM | | | | | | | |
| | | GMIDPSDSYTSYSPSFQ | | | | | | | |
| | | GHVTISADKSISTAYLQ | | | | | | | |
| | | WSSLKASDTAMYYCAR | | | | | | | |
| | | LHYYNSEEFDVWGQGT | | | | | | | |
| | | LVTVSS | | | | | | | |
| mAb11- | VL | EIVLTQSPGTLSLSPGE | 1183 | RASQSVSSS | 1184 | GQSSRTR | 1185 | QQFGDDQLFT | 1186 |
| 1493 | | RATLSCRASQSVSSSYL | | YLA | | | | | |
| | | AWYQQKPGQAPRLLIY | | | | | | | |
| | | GQSSRTRGIPDRFSGS | | | | | | | |
| | | GSGTDFTLTISRLEPED | | | | | | | |
| | | FAVYYCQQFGDDQLFT | | | | | | | |
| | | FGQGTKLEIK | | | | | | | |
| mAb11- | VH | EVQLVQSGAEVKKPGE | 1187 | TSWIV | 1188 | MIDPSDSYTSYSPSFQ | 1189 | LHYYNSEEFDV | 1190 |
| 1494 | | SLRISCKGSGYSFSTSW | | | | G | | | |
| | | IVWVRQMPGKGLEWM | | | | | | | |
| | | GMIDPSDSYTSYSPSFQ | | | | | | | |
| | | GHVTISADKSISTAYLQ | | | | | | | |
| | | WSSLKASDTAMYYCAR | | | | | | | |
| | | LHYYNSEEFDVWGQGT | | | | | | | |
| | | MVTVSS | | | | | | | |
| mAb11- | VL | EIVLTQSPGTLSLSPGE | 1191 | RASQSVSSS | 1192 | GQSSRTR | 1193 | QQFGDDQLFT | 1194 |
| 1494 | | RATLSCRASQSVSSSYL | | YLA | | | | | |
| | | AWYQQKPGQAPRLLIY | | | | | | | |
| | | GQSSRTRGIPDRFSGS | | | | | | | |
| | | GSGTDFTLTISRLEPED | | | | | | | |
| | | FAVYYCQQFGDDQLFT | | | | | | | |
| | | FGQGTKLEIK | | | | | | | |

### Example 7a: Binning of Anti-FIX/FIXa Stimulating Antibodies

Antibodies capable of stimulating the enzymatic activity of FIXa towards FX were analysed in binning experiments to determine the binding characteristics for the identified antibodies using the method described below. Both parent antibodies and engineered variants were analysed, and compared to other known antibodies.

Method for Binning of Antibodies

Binning experiments were performed using Octet fortebio systems (HTX, Red384), based on the principle of Bio-Layer Interferometry, and equipped with streptavidin sensors (Pall Life Sciences, Menlo Park, Calif.), and using 8-channel mode. The binning assays were performed using a modified in-tandem setup. Briefly, (1) 370 nM randomly biotinylated human FIXa (biotinylated using NHS-d-biotin from Sigma H1759 and human FIXa obtained from Haematologic Technologies, Essex Junction, VT) was captured by streptavidin tips (dip and read biosensors Part NO:18-5019, Pall Life Sciences, Menlo Park, Calif.) for 5 minutes, (2) 330 nM of a first bivalent anti-FIXa antibody was then offered to the streptavidin tips, and incubated for 10 minutes until the biotinylated FIXa were fully saturated and (3) the tips were then offered to an equimolar solution of 330 nM of the first antibody and 330 nM of a second bivalent anti-FIXa antibody for 5 minutes. This modified in-tandem set-up, with inclusion of an equimolar concentration of the first antibody in the second antibody incubation step, were preferred due to the very low affinity (and fast koff rates) of some of the used antibodies. Unspecific binding were evaluated by including an unrelated human IgG4 bivalent antibody as first and second antibody control. As seen in Table 4, where the response values from step (3) are reported, the analysis identified three different bins, represented by the bivalent anti-FIXa antibodies 224F3 (mAb01-1582), mAb01-1767 and mAb01-2434 (ACE910 anti-FIX(a) arms). From Table 4 it is also clear, that the engineered antibodies mAb01-9933, mAb01-9978, mAb01-9985, and mAb01-9994 show the same binning pattern as their parent antibody mAb01-1767 and consequently belong to BinB (as indicated in Table 4).

TABLE 4
Interferometry response values

| | 1st antibody | | | |
|---|---|---|---|---|
| | 224F3 | mAb01-1767 | mAb01-2434 | hIgG4 |
| 2nd antibody | (BinA) | (BinB) | (BinC) | (control) |
| 224F3 | 0.11 | 1.60 | 1.69 | 2.11 |
| mAb01-1767 | 0.95 | 0.02 | 1.11 | 1.41 |
| mAb01-2434 | 0.41 | 0.49 | 0.14 | 0.85 |
| mAb01-9933 | 0.34 | 0.02 | 0.46 | 0.59 |
| mAb01-9978 | 0.24 | 0.01 | 0.34 | 0.40 |
| mAb01-9985 | 0.44 | 0.02 | 0.60 | 0.83 |
| mAb01-9994 | 0.40 | 0.01 | 0.56 | 0.75 |
| hIgG4 (control) | 0.08 | −0.02 | −0.04 | −0.03 |

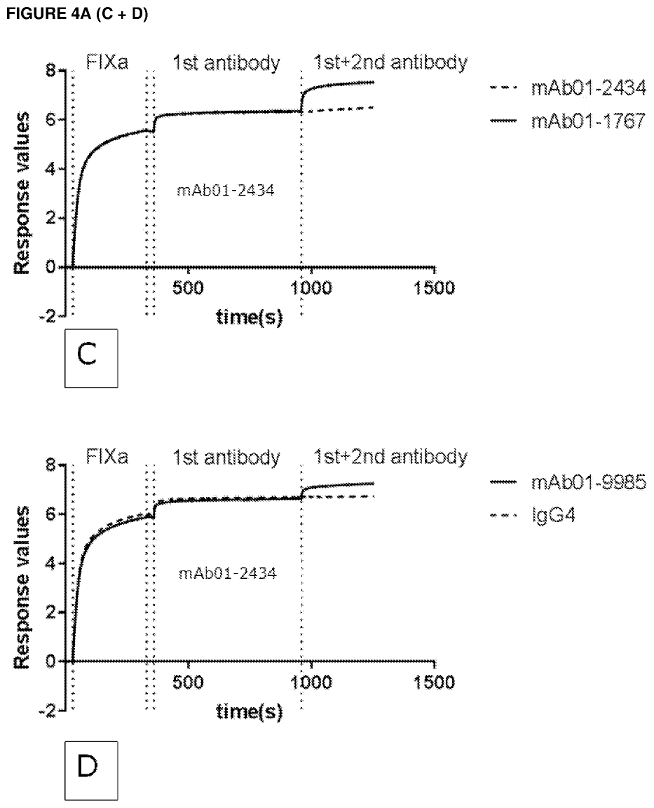

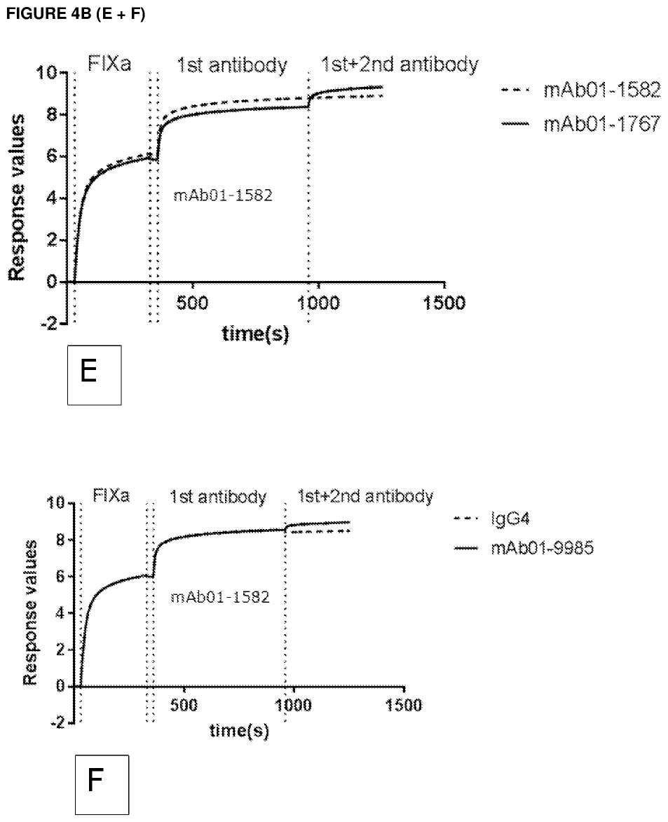

As seen from Table 4, the control antibody human IgG4 was not able to bind to FIXa when used as second antibody (Table 4 last row) and did not prevent binding of the second antibody to FIXa when used as first antibody (Table 4 last column). 224F3 and mAb01-2434 had self-competition response values higher that zero, namely 0.11 and 0.14, respectively, still well beyond the lowest response values among the second antibodies belonging to a different bin, e.g. 0.24 for mAb01-9978 (2^{nd} antibody)/224F3 (1^{st} antibody) and 0.34 for the mAb01-9978 (2^{nd} antibody)/mAb01-2434 (1^{st} antibody). Some examples of the full binding curves are shown in FIGS. 4A and 4B (C, D, E and F)

### Example 7b: Binning of Anti-FX/FXa Stimulating Antibodies

Anti-FX(a) antibodies were analysed in binning experiments to determine the binding characteristics for the identified antibodies using the method described below. Both parent antibodies and engineered variants were analysed, and compared to other known antibodies.

Method for Binning of Antibodies

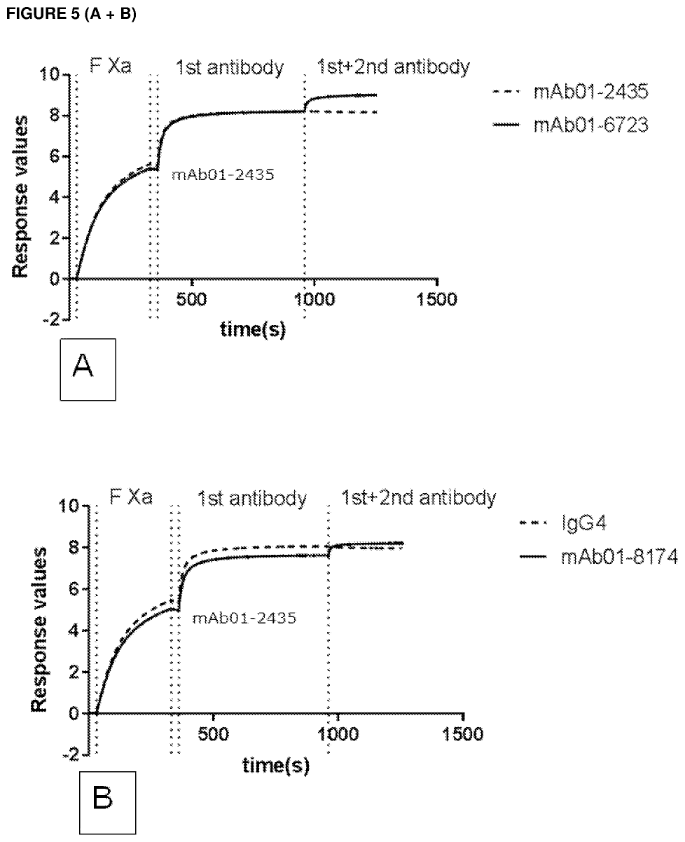

Binning experiments were performed using Octet fortebio systems (HTX, Red384) equipped with streptavidin sensors (Pall Life Sciences, Menlo Park, Calif.), and using 8-channel mode (Red384 and HTX). The binning assays were performed using a modified in-tandem setup. Briefly, (1) 363 nM randomly biotinylated human FXa (obtained from Haematologic technologies and biotinylated using NHS-d-biotin) was captured by streptavidin tips for 5 minutes (dip and read biosensors Part NO:18-5019, Pall Life Sciences, Menlo Park, Calif.), (2) 330 nM of a first bivalent anti-FX(a) antibody was then offered to the streptavidin tips, and incubated for 10 minutes until the biotinylated FXa were fully saturated and (3) the tips were then offered to an equimolar solution of 330 nM of the first antibody and 330 nM of a second bivalent anti-FX(a) antibody for 5 minutes. The set-up with inclusion of an equimolar concentration of the first antibody in the second antibody incubation step, were preferred due to the very low affinity (and fast koff rates) of some of the used antibodies. Unspecific binding were evaluated by including an unrelated human IgG4 bivalent antibody as first and second antibody control. As seen in Table 5, where the response values from step (3) are reported, the analysis identified two different bins, represented by the bivalent anti-FX(a) antibodies mAb01-2435 (ACE910 anti-FX(a) arms) and mAb01-6723. From Table 5 it is also clear, that the engineered antibodies mAb01-8174 and mAb01-9772 show the same binning pattern as their parent antibody mAb01-6723 and consequently belong to Bin2 (as indicated in Table 5). Some examples of the full binding curves are shown in FIG. 5 (A and B).

TABLE 5
Interferometry response values

| | 1st antibody | | |
|---|---|---|---|
| | mAb01-2435 | mAb01-6723 | hIgG4 |
| 2nd antibody | (Bin1) | (Bin2) | (control) |
| mAb01-2435 | −0.05 | 0.85 | 2.63 |
| mAb01-6723 | 0.79 | −0.02 | 2.59 |
| mAb01-8174 | 0.62 | 0.05 | 2.29 |
| mAb01-9772 | 0.22 | −0.01 | 1.49 |
| hIgG4 (control) | −0.08 | −0.03 | −0.09 |

### Example 8: Crystallization and Epitope/Paratope Mapping of Anti-FIX(a) Antibodies Using X-Ray Crystallography

Crystallization and Epitope/Paratope Mapping of Anti-FIX(a) Antibody mAb01-9994

Crystallization

Fab fragment corresponding to mAb01-9994 was mixed in a 1:1 molar ratio with human EGR-CK-inhibited Factor IXa Gla-domainless (wild-type) bacterial expression, Lot #hGDFIXAWTEGR_05 (purchased from Cambridge ProteinWorks) and crystals of the Fab/FIXa complex were grown using the sitting drop vapour diffusion technique at 18° C. A protein solution of 100 nl 5.5 mg/ml complex in 20 mM Tris-HCl, pH 7.4, 50 mM NaCl, and 2.5 mM CaCl₂) was mixed with 100 nl of 0.1M Bicine, pH 9.0, 2% (v/v) 1,4-dioxane, 10% PEG 20000 as precipitant and incubated over 60 μl precipitant.

Diffraction Data Collection

The crystal was cryo protected by addition of 1 μl of precipitant added 20% of ethylene glycol to the crystallization drop prior to flash cooling in liquid nitrogen. Diffraction data were collected at 100K at the Diamond Light Source beamline i03 (0.9763 Å wavelength) using a Pilatus3 6M pixel detector from Dectris. Autoindexing, integration and scaling of the data were performed with programmes from the XDS package (diffracting data statistics are summarised in Table 6).

Structure Determination and Refinement

The asymmetric unit contains one Fab:FIXa complex as judged from Matthews coefficient analysis. The structure was determined by molecular replacement using Phaser as implemented in the programme suite Phenix using a structure of a predetermined Fab:FIXa complex as search model. The correct amino acid sequence was model built using COOT and thereafter the structure was refined using steps of Phenix refinement and manual rebuilding in COOT. The refinement statistics are found in Table 6.

TABLE 6
Data collection and refinement statistics

| Wavelength (Å) | 0.9763 | |
|---|---|---|
| Resolution range (Å) | 43.06-2.7 | (2.797-2.7) |
| Space group | P4₃2₁2 | |
| Unit cell (Å, deg) | 87.65 87.65 231.8 90 90 90 | |
| Total reflections | 327518 | (33655) |
| Unique reflections | 25718 | (2511) |
| Multiplicity | 12.7 | (13.4) |
| Completeness (%) | 99.94 | (100.00) |
| Mean I/sigma(I) | 13.21 | (1.28) |
| Wilson B-factor (Å²) | 77.47 | |
| R-merge | 0.15 | (2.05) |
| R-meas | 0.1565 | (2.13) |
| R-pim | 0.04391 | (0.5757) |
| CC1/2 | 0.998 | (0.447) |
| CC* | 1 | (0.786) |
| Reflections used in refinement | 25715 | (2511) |
| Reflections used for R-free | 968 | (93) |
| R-work | 0.1978 | (0.3407) |
| R-free | 0.2494 | (0.4171) |
| CC(work) | 0.933 | (0.554) |
| CC(free) | 0.988 | (0.575) |

| | |
|---|---|
| Number of non-hydrogen atoms | 5597 |
| macromolecules | 5540 |
| ligands | 25 |
| solvent | 32 |
| Protein residues | 719 |
| RMS(bonds) (Å) | 0.012 |
| RMS(angles) (deg) | 1.92 |
| Ramachandran favored (%) | 90.24 |
| Ramachandran allowed (%) | 7.92 |
| Ramachandran outliers (%) | 1.84 |
| Rotamer outliers (%) | 0.00 |
| Clashscore | 12.93 |
| Average B-factor (Å²) | 82.66 |
| Macromolecules | 82.77 |
| Ligands | 85.42 |
| Solvent | 61.00 |

Statistics for the highest-resolution shell are shown in parentheses.

Determination of the Epitope of mAb01-9994

The epitope of mAb01-9994, defined as FIX(a) residues characterized by having a heavy atom (i.e. a non-hydrogen atom) within a distance of 3.5 Å from a heavy atom in the Fab, comprises the following residues L337, R338, T340, K341 and T343, according to SEQ ID NO:1.

Determination of the Paratope of mAb01-9994

The paratope for mAb01-9994 defined as Fab residues characterized by having a heavy atom (i.e. a non-hydrogen atom) within a distance of 3.5 Å from a heavy atom in FIXa, comprises the residues (consecutive numbering): H30, D31, W53, D56, S102, S104, Y106 and N107 in the heavy chain variable domain (SEQ ID NO:67) and residues Y91 and S92 in the light chain variable domain (SEQ ID NO:71).

Residues in bold are located in the CDR sequences, as defined using the Kabat definition, while the remaining paratope residues H30 (in the heavy chain variable domain) is a framework residue.

Crystallization and Epitope/Paratope Mapping of Anti-FIX/FIXa Antibody mAb01-9933

Crystallization

Fab fragment corresponding to mAb01-9933 was mixed in a 1:1 molar ratio with human EGR-CK-inhibited Factor IXa Gla-domainless (wild-type) bacterial expression, Lot #hGDFIXAWTEGR_05 (purchased from Cambridge ProteinWorks) and crystals of the Fab/FIXa complex were grown using the sitting drop vapour diffusion technique at 18° C. A protein solution of 150 nl 8.1 mg/ml complex in 20 mM Tris-HCl, pH 7.4, 50 mM NaCl, and 2.5 mM CaCl₂ was mixed with 50 nl of 0.1 M Bis-Tris, pH 6.5, 20% (w/v) PEG 5000 MME as precipitant and incubated over 60 μl precipitant.

Diffraction Data Collection

The crystal was cryo protected by addition of 1 μl of precipitant added 20% of ethylene glycol to the crystallization drop prior to flash cooling in liquid nitrogen. Diffraction data were collected at 100K at the Diamond Light Source beamline i03 (0.9763 Å wavelength) using a Pilatus3 6M pixel detector from Dectris. Autoindexing, integration and scaling of the data were performed with programmes from the XDS package (diffracting data statistics are summarised in Table 7).

Structure Determination and Refinement

The asymmetric unit contains one Fab:FIXa complex as judged from Matthews coefficient analysis. The structure was determined by molecular replacement using Phaser as implemented in the programme suite Phenix using a structure of a predetermined Fab:FIXa complex as search model. The correct amino acid sequence was model built using COOT and thereafter the structure was refined using steps of Phenix refinement and manual rebuilding in COOT. The refinement statistics are found in Table 7.

TABLE 7
Data collection and refinement statistics

| Wavelength (Å) | 0.9763 | |
|---|---|---|
| Resolution range (Å) | 47.16-2.4 | (2.486-2.4) |
| Space group | P 4₃2₁2 | |
| Unit cell (Å, deg) | 85.7 85.7 225.95 90 90 90 | |
| Total reflections | 433016 | (39929) |
| Unique reflections | 33886 | (3317) |
| Multiplicity | 12.8 | (12.0) |
| Completeness (%) | 99.95 | (99.97) |
| Mean I/sigma(I) | 13.85 | (1.41) |
| Wilson B-factor | 53.78 | |
| R-merge | 0.1353 | (1.658) |
| R-meas | 0.1412 | (1.732) |
| R-pim | 0.03956 | (0.4966) |
| CC1/2 | 0.999 | (0.591) |
| CC* | 1 | (0.862) |
| Reflections used in refinement | 33879 | (3317) |
| Reflections used for R-free | 1279 | (128) |
| R-work | 0.2171 | (0.2721) |
| R-free | 0.2623 | (0.3245) |
| CC(work) | 0.957 | (0.764) |
| CC(free) | 0.991 | (0.670) |

| | |
|---|---|
| Number of non-hydrogen atoms | 5624 |
| macromolecules | 5497 |
| Ligands | 25 |
| Solvent | 102 |
| Protein residues | 715 |
| RMS(bonds) (Å) | 0.011 |
| RMS(angles) (deg) | 1.36 |
| Ramachandran favored (%) | 90.70 |
| Ramachandran allowed (%) | 7.58 |
| Ramachandran outliers (%) | 1.72 |
| Rotamer outliers (%) | 0.49 |
| Clashscore | 9.45 |
| Average B-factor (Å²) | 64.71 |
| Macromolecules | 64.92 |
| Ligands | 60.38 |
| Solvent | 54.67 |

Statistics for the highest-resolution shell are shown in parentheses.

Determination of the Epitope of mAb01-9933

The epitope of mAb01-9933, defined as FIX(a) residues characterized by having a heavy atom (i.e. a non-hydrogen atom) within a distance of 3.5 Å from a heavy atom in the Fab, comprises the following residues L337, R338, T340, and K341, according to SEQ ID NO:1.

Determination of the Paratope of mAb01-9933

Additionally, the paratope for mAb01-9933 defined as Fab residues characterized by having a heavy atom (i.e. a non-hydrogen atom) within a distance of 3.5 Å from a heavy atom in FIXa, comprises the residues (consecutive numbering): D30, D31, W53, S102, S104, N107 in the heavy chain variable domain (SEQ ID NO:35) and residues: Y91 and S92 in the light chain variable domain (SEQ ID NO:39).

Residues in bold are located in the CDR sequences, as defined using the Kabat definition, while the remaining paratope residues D30 (in the heavy chain variable domain) is a framework residue.

### Example 8a: Crystallization and Epitope/Paratope Mapping of Anti-FIX(a) Antibody mAb01-9985

Crystallization

Fab fragment corresponding to mAb01-9985 was mixed in a 1:1 molar ratio with human EGR-CK-inhibited Factor IXa Gla-domainless (wild-type) bacterial expression, Lot #hGDFIXAWTEGR_11 (purchased from Cambridge ProteinWorks). The complex was subjected to size exclusion chromatography on a HiLoad 16/60 Superdex 200 pg column (GE Healthcare) run with 20 mM Hepes, pH 7.5, 140 mM NaCl, 1 mM CaCl₂) buffer. The fractions containing the Fab/FIXa complex were pooled and concentrated to 10.1 mg/ml. Crystals of the Fab/FIXa complex were grown using the microseed matrix screening technique as described in D'Arcy et al. (2014) Acta Crystallographica Section F 70, 1117-1126 using sitting drop vapour diffusion at 18° C. The crystal used was grown using a protein solution of 200 nl 10.1 mg/ml complex in 20 mM Hepes, pH 7.4, 140 mM NaCl, 1 mM CaCl₂ mixed with 100 nl seed stock and 300 nl of 2 M ammonium sulphate, 0.1 M Hepes, pH 7.5 as precipitant and incubated over 80 μl precipitant. The seed stock was prepared from crystals of the Fab fragment corresponding to mAb01-9933 in complex with human EGR-CK-inhibited Factor IXa Gla-domainless (wild-type).

Diffraction Data Collection

The crystal was cryo protected by addition of 1.5 μl of precipitant added 20% of ethylene glycol to the crystallization drop prior to flash cooling in liquid nitrogen. Diffraction data were collected at 100K at the Swiss Light Source beamline X10SA (1.00 Å wavelength) using a Pilatus3 6M pixel detector from Dectris. Autoindexing, integration and scaling of the data were performed with programmes from the XDS package (diffracting data statistics are summarised in Table 7a).

Structure Determination and Refinement

The asymmetric unit contains one Fab:FIXa complex as judged from Matthews coefficient analysis. The structure was determined by molecular replacement using Phaser as implemented in the programme suite Phenix using a structure of a predetermined Fab:FIXa complex as search model. The correct amino acid sequence was model built using COOT and thereafter the structure was refined using steps of Phenix refinement and manual rebuilding in COOT. The refinement statistics are found in Table 7a.

TABLE 7a
Data collection and refinement statistics

| Wavelength (Å) | 1.00 | |
|---|---|---|
| Resolution range (Å) | 48.51-3.105 | (3.216-3.105) |
| Space group | P 4₁22 | |
| Unit cell (Å, deg) | 97.03 97.03 254.51 90 90 90 | |
| Total reflections | 295893 | (28675) |
| Unique reflections | 22811 | (2201) |
| Multiplicity | 13.0 | (13.0) |
| Completeness (%) | 99.80 | (99.19) |
| Mean I/sigma(I) | 4.30 | (0.95) |
| Wilson B-factor (Å²) | 50.79 | |
| R-merge | 0.692 | (2.568) |
| R-meas | 0.7205 | (2.672) |
| R-pim | 0.1985 | (0.7295) |
| CC1/2 | 0.957 | (0.476) |
| CC* | 0.989 | (0.803) |
| Reflections used in refinement | 22786 | (2193) |
| Reflections used for R-free | 548 | (53) |
| R-work | 0.2240 | (0.3283) |
| R-free | 0.2794 | (0.3814) |
| CC(work) | 0.940 | (0.725) |
| CC(free) | 0.928 | (0.614) |

| | |
|---|---|
| Number of non-hydrogen atoms | 5690 |
| macromolecules | 5595 |
| ligands | 95 |
| Protein residues | 726 |
| RMS(bonds) (Å) | 0.012 |
| RMS(angles) (deg) | 1.70 |
| Ramachandran favored (%) | 97.21 |
| Ramachandran allowed (%) | 2.65 |
| Ramachandran outliers (%) | 0.14 |
| Rotamer outliers (%) | 0.16 |
| Clashscore | 3.58 |
| Average B-factor (Å²) | 43.84 |
| Macromolecules | 43.53 |
| Ligands | 61.76 |

Statistics for the highest-resolution shell are shown in parentheses.

Determination of the Epitope of mAb01-9985

The epitope of mAb01-9985, defined as FIX(a) residues characterized by having a heavy atom (i.e. a non-hydrogen atom) within a distance of 3.5 Å from a heavy atom in the Fab, comprises the following residues L337, R338, S339, T340, and K341, according to SEQ ID NO:1.

Determination of the Paratope of mAb01-9985

The paratope for mAb01-9985 defined as Fab residues characterized by having a heavy atom (i.e. a non-hydrogen atom) within a distance of 3.5 Å from a heavy atom in FIXa, comprises the residues (consecutive numbering): H30, D31, W53, S102, S104, Y106 and N107 in the heavy chain variable domain (SEQ ID NO:51) and residues Y91 and S92 in the light chain variable domain (SEQ ID NO:55). Residues in bold are located in the CDR sequences, as defined using the Kabat definition, while the remaining paratope residue H30 (in the heavy chain variable domain) is a framework residue.

### Example 9: Crystallization and Epitope/Paratope Mapping of Anti-FX Antibodies Using X-Ray Crystallography

Crystallization and Epitope/Paratope Mapping of Anti-FXa Antibody mAb01-8174

Crystallization

Fab fragment corresponding to mAb01-8174 was mixed in a 1:1 molar ratio with human EGR-CK-inhibited Factor Xa Gla-domainless (wild-type) bacterial expression, Lot #hGDFXAEGR_026 (purchased from Cambridge ProteinWorks) and crystals of the Fab/FXa complex were grown using the sitting drop vapour diffusion technique at 18° C. A protein solution of 150 nl 4.7 mg/ml complex in 20 mM Tris-HCl, pH 7.4, 50 mM NaCl, and 2.5 mM CaCl₂) was mixed with 50 nl of 0.2 M sodium acetate, 0.1 M sodium cacodylate, pH 6.5, 18% (w/v) PEG 8000 as precipitant and incubated over 60 μl precipitant.

Diffraction Data Collection

The crystal was cryo protected by addition of 1 μl of precipitant added 20% of ethylene glycol to the crystallization drop prior to flash cooling in liquid nitrogen. Diffraction data were collected at 100K at the Swiss Light Source beamline X06DA (1.00 Å wavelength) using a Pilatus2M pixel detector from Dectris. Autoindexing, integration and scaling of the data were performed with programmes from the XDS package (diffracting data statistics are summarised in Table 8).

Structure Determination and Refinement

The asymmetric unit contains two Fab:FXa complexes as judged from Matthews coefficient analysis. The structure was determined by molecular replacement with Molrep as implemented in the programme suite CCP4 using structures of predetermined Fab:FXa complexes as search models. The correct amino acid sequence for the Fab was model built using COOT and thereafter the structure was refined using steps of Phenix refinement and manual rebuilding in COOT. The refinement statistics are found in Table 8.

TABLE 8
Data collection and refinement statistics

| Wavelength (Å) | 1.000 | |
|---|---|---|
| Resolution range (Å) | 44.12-2.6 | (2.693-2.6) |
| Space group | P2₁ | |
| Unit cell (Å, deg) | 63.13 105.25 145.78 90 89.995 90 | |
| Total reflections | 302807 | (30703) |
| Unique reflections | 58767 | (5896) |
| Multiplicity | 5.2 | (5.2) |
| Completeness (%) | 99.90 | (99.97) |
| Mean I/sigma(I) | 12.31 | (1.56) |
| Wilson B-factor (Å²) | 50.13 | |
| R-merge | 0.1209 | (1.024) |
| R-meas | 0.1346 | (1.138) |
| R-pim | 0.05869 | (0.4933) |
| CC1/2 | 0.996 | (0.523) |
| CC* | 0.999 | (0.829) |
| Reflections used in refinement | 58761 | (5896) |
| Reflections used for R-free | 1999 | (202) |
| R-work | 0.2033 | (0.3027) |
| R-free | 0.2623 | (0.3140) |
| CC(work) | 0.857 | (0.463) |
| CC(free) | 0.788 | (0.347) |

| | |
|---|---|
| Number of non-hydrogen atoms | 11641 |
| macromolecules | 11145 |
| ligands | 52 |
| solvent | 444 |
| Protein residues | 1447 |
| RMS(bonds) | 0.011 |
| RMS(angles) | 1.70 |
| Ramachandran favored (%) | 95.74 |
| Ramachandran allowed (%) | 3.70 |
| Ramachandran outliers (%) | 0.56 |
| Rotamer outliers (%) | 0.32 |
| Clashscore | 22.55 |
| Average B-factor | 46.64 |
| macromolecules | 46.94 |
| ligands | 46.66 |
| solvent | 39.14 |
| Twin refinement | h, −k, −l |

Statistics for the highest-resolution shell are shown in parentheses.

Determination of Epitope of mAb01-8174

The epitope of mAb01-8174, defined as FXa residues characterized by having a heavy atom (i.e. a non-hydrogen atom) within a distance of 3.5 Å from a heavy atom in the Fab in one or both of the complexes in the asymmetric unit, comprises the following residues: E103, Q104, V108, R113, T116, L117, D119, I125, T127, E228, F229, Y230, E266, R287, P291, I292, P304, L419, K420, D423, R424, M426, K427 and T428 according to SEQ ID NO:2.

Determination of Paratope of mAb01-8174

The paratope for mAb01-8174 defined as Fab residues characterized by having a heavy atom (i.e. a non-hydrogen atom) within a distance of 3.5 Å from a heavy atom in FXa in one or both of the complexes in the asymmetric unit, comprises the residues (consecutive numbering): K23, S25, G26, Y27, F29, W33, D52, S54, D55, F57, S77, H100, Y101, Y102, N103, S104 in the heavy chain variable domain (SEQ ID NO:467) and residues V29, S30, S31, Y33, Y50, Q52, S54, R55, R57 and D94 in the light chain variable domain (SEQ ID NO:471).

Residues in bold are located in the CDR sequences, as defined using the Kabat definition, while the remaining paratope residues K23, S25, G26, Y27, F29 and S77 (in the heavy chain variable domain) and Y50 (in light chain variable domain) are framework residues.

Crystallization and Epitope/Paratope Mapping of Anti-FXa Antibody mAb01-9772

Fab fragment corresponding to mAb01-9772 was mixed in a 1:1 molar ratio with human EGR-CK-inhibited Factor Xa Gla-domainless (wild-type) bacterial expression, Lot #hGDFXAEGR_026 (purchased from Cambridge ProteinWorks) and crystals of the Fab/FXa complex were grown using the sitting drop vapour diffusion technique at 18° C. A protein solution of 150 nl 3.7 mg/ml complex in 20 mM Tris-HCl, pH 7.4, 50 mM NaCl, and 2.5 mM CaCl₂) was mixed with 50 nl of 0.2 M sodium formate, 20% (w/v) PEG 3350 as precipitant and incubated over 60 μl precipitant.

Diffraction Data Collection

The crystal was cryo protected by addition of 1 μl of precipitant added 20% of ethylene glycol to the crystallisation drop prior to flash cooling in liquid nitrogen. Diffraction data were collected at 100K at the Swiss Light Source beamline X06DA (1.00 Å wavelength) using a Pilatus2M pixel detector from Dectris. Autoindexing, integration and scaling of the data were performed with programmes from the XDS package (diffracting data statistics are summarised in Table 9).

Structure Determination and Refinement

The asymmetric unit contains two Fab:FXa complexes as judged from Matthews coefficient analysis. The structure was determined by molecular replacement with Molrep as implemented in the programme suite CCP4 using structures of predetermined Fab:FXa complexes as search models. The correct amino acid sequence for the Fab was model built using COOT and thereafter the structure was refined using steps of Phenix refinement and manual rebuilding in COOT. The refinement statistics are found in Table 9.

TABLE 9
Data collection and refinement statistics

| Wavelength (Å) | 1.000 | |
|---|---|---|
| Resolution range | 48.33-3.15 | (3.263-3.15) |
| Space group | P 2₁ | |
| Unit cell (Å, deg) | 62.34 104.72 144.98 90 90.048 90 | |
| Total reflections | 102910 | (10275) |
| Unique reflections | 31847 | (3182) |
| Multiplicity | 3.2 | (3.2) |
| Completeness (%) | 98.16 | (98.45) |
| Mean I/sigma(I) | 8.51 | (1.45) |
| Wilson B-factor (Å²) | 74.36 | |
| R-merge | 0.126 | (0.7224) |
| R-meas | 0.1512 | (0.8628) |
| R-pim | 0.08262 | (0.4665) |
| CC1/2 | 0.994 | (0.571) |
| CC* | 0.998 | (0.853) |
| Reflections used in refinement | 31824 | (3182) |
| Reflections used for R-free | 1083 | (113) |
| R-work | 0.2163 | (0.2810) |
| R-free | 0.2929 | (0.2997) |
| CC(work) | 0.849 | (0.374) |
| CC(free) | 0.736 | (0.325) |

| | |
|---|---|
| Number of non-hydrogen atoms | 11190 |
| macromolecules | 11138 |
| ligands | 52 |
| Protein residues | 1446 |
| RMS(bonds) | 0.013 |
| RMS(angles) | 1.98 |
| Ramachandran favored (%) | 95.03 |
| Ramachandran allowed (%) | 4.20 |
| Ramachandran outliers (%) | 0.77 |
| Rotamer outliers (%) | 0.00 |
| Clashscore | 26.08 |
| Average B-factor (Å²) | 73.74 |
| macromolecules | 73.78 |
| ligands | 65.77 |
| Twin refinement | h, −k, −l |

Statistics for the highest-resolution shell are shown in parentheses.

Determination of Epitope of mAb01-9772

The epitope of mAb01-9772, defined as FXa residues characterized by having a heavy atom (i.e. a non-hydrogen atom) within a distance of 3.5 Å from a heavy atom in the Fab in one or both of the complexes in the asymmetric unit, comprises the following residues: E103, Q104, V108, R113, T116, L117, A118, D119, I125, T127, S227, E228, Y230, R287, I292, L303, P304, L419, K420, D423, R424, M426, K427 and T428 according to SEQ ID NO:2.

Determination of Paratope of mAb01-9772

The paratope for mAb01-9772 defined as Fab residues characterized by having a heavy atom (i.e. a non-hydrogen atom) within a distance of 3.5 Å from a heavy atom in FXa in one or both of the complexes in the asymmetric unit, comprises the residues (consecutive numbering): K23, G24, S25, G26, Y27, W33, D52, S54, D55, Y57, S77, L99, H100, Y101, Y102, N103 and S104 in the variable heavy chain domain (SEQ ID NO:483) and residues S30, S31, Y33, Y50, Q52, S54, R55, R57, Y92 and D94 in the light chain variable domain (SEQ ID NO:487).

Residues in bold are located in the CDR sequences, as defined using the Kabat definition, while the remaining paratope residues K23, G24, S25, G26, Y27 and S77 (in the heavy chain variable domain) and Y50 (in light chain variable domain) are framework residues.

### Example 10: Identification of Hot-Spot Residues on FIX

In order to determine residues critical for the interaction (referred to as hot-spot) between the anti-FIX/FIXa mAb01-9994 and mAb01-9985 and FIX, a set of FIX variants was selected based on Fab/FIXa structure of the Fab fragment of mAb01-9994 and FIXa. As detailed below the selected FIX variants were transiently expressed in mammalian cells, purified and characterized with respect to their binding to monovalent variants of mAb01-9994 and mAb01-9985 using Surface Plasmon Resonance (SPR).

Generation of FIX Mutants

A DNA plasmid, suitable for transient mammalian expression, was constructed with an expression cassette encoding amino acids residues 1-461 of human FIX (uniprot P00740, except for a T194A mutation according to the UNIPROT numbering, corresponding to T148A of SEQ ID NO:1) directly followed by six Histidines (6× His-tag, for affinity purification). The secreted, mature FIX protein chain produced using this construct is identical to the A148 allelic form of human FIX (Anson et al. EMBO J. 1984 3:1053-1060, McGraw et al., Proc Natl Acad Sci USA. 1985 82:2847-2851) except for the addition of the C-terminal His-tag. Using the construct as template, selected mutations were introduced by PCR. For each single-point mutation listed in Table 10, a forward primer containing the desired amino acid change and a reverse primer without amino acid mutations were designed. These primers were used in a standard PCR reaction with the vector described above as template to amplify the entire vector sequence. Ligation-free cloning was used to join the ends of the resulting amplified DNA fragment into a circular expression plasmid using overlap sequences introduced by the forward and the reverse primers.

The circularized plasmids were transformed into E. coli cells, grown on selective agar plates to form colonies, and the colonies used to start liquid E. coli cultures. After overnight growth of the E. coli cultures, plasmid preparations were performed and the mutants identified by DNA sequencing.

Recombinant protein production was performed by transfecting expi293F cells growing in suspension culture in Expi293 Expression™ medium (ThermoFisher Scientific, cat #A1435101) using the ExpiFectamine™ 293 Transfection Kit (ThermoFisher Scientific, cat #A14525) and plasmid DNA encoding each of the desired variants as well as wild-type FIX (corresponding to SEQ ID NO:1 with C-terminal His-tag). Vitamin K was added to a final concentration of 5 mg/mL at the time of transfection. Transfection Enhancers 1 and 2 from the ExpiFectamine™ 293 Transfection Kit were added the day after transfection. The cell cultures were harvested 5 days after transfection by centrifugation.

The C-terminal His-tag on each FIX variant was used for batch protein purification in a multi-well, robotic setup. Briefly, the harvested cell culture supernatants were adjusted to binding conditions, mixed with Ni Sepharose 6 Fast Flow affinity purification resin (GE Healthcare, cat #17-5318-02, 50 μl sedimented resin/ml cell culture medium) and incubated while shaking for 20 minutes. The resin/supernatant mixes were then transferred to a filter plate and the liquid drawn through the filter plate by application of vacuum. The resin remaining in the filter plate was washed three times before elution in a high-imidazole buffer.

Concentration determination of the purified protein solutions was performed by ELISA, using an anti-FIX antibody for detection and high-purity recombinant wild-type FIX for standard curves.

The FIX variants were characterized with respect to their binding to mAb01-9994 and mAb01-9985 using surface plasmon resonance (SPR) by capturing the FIX variant via the C-terminal His-tag. To avoid avidity effects, i.e. ensure a 1:1 interaction, one-armed (OA) variants of mAb01-9994 and mAb01-9985 (prepared as described in Example 5), were used as analytes.

SPR anayses were carried out on Biacore T200 instruments (Biacore AB, Uppsala, Sweden). For the experiments on the T200 instrument the following conditions were applied: measurements were conducted at a temperature of 25° C. Anti-His antibody at 25 μg/ml (R&D Systems, catalogue #MAB050) was immobilized on a CM5 sensor chip using standard amine coupling chemistry. Anti-FIX variants at 25 nM were injected at a flow rate of 10 μl/min for 1 min and were captured via their His-tag by the immobilized anti-His antibody.

Subsequently, 20 μM (with 2.5 fold dilution) of OA mAb01-9994 and OA mAb01-9985 were injected at a flow rate of 50 μl/min for 3 min to allow for binding to captured FIX variant followed by a 3 min buffer injection allowing for dissociation of the monovalent anti-FIX antibodies. The running buffer used was 20 mM Tris, 150 mM NaCl, 5 mM CaCl₂, 0.05% Tween-20, 10 mg/ml BSA, pH 7.4. This was also used for dilution of anti-FIX antibody and FIX samples. Regeneration of the chip was achieved using 10 mM Glycine pH 2.0. Binding data were analysed according to a 1:1 model using BiaEvaluation 4.1 supplied by the manufacturer (Biacore AB, Uppsala, Sweden).

Binding data are reported as % binding of the antibody to the FIX variant relative to binding of the antibody to wild-type FIX calculated according to the formula:

Binding (%)=100%×[(R_{max_Ab,FIX_var})/(R_{max_FIXvar})]/[(R_{max_Ab,FIX_wt})/(R_{max_FIXwt})]

where R_{max_FIXvar} and R_{max_FIXwt} represent capture level (RU) of FIX variant and wild-type FIX, respectively, and where R_{max_Ab,FIX_var} and R_{max_Ab,FIX_wt} represent Rmax (RU) of the 20 μM antibody to captured FIX variant and wild-type FIX, respectively. Results are shown in Tables 10 and 11.

TABLE 10
Results from SPR analysis (mAb01-9994)
Results from hot-spot analysis of OA variant of mAb01-9994

| Residue# | FIX variant | Binding (%) |
|---|---|---|
| 301 | K301A | 89 |
| 332 | D332S | 41 |
| 332 | D332A | 41 |
| 333 | R333A | 55 |
| 334 | A334L | 38 |
| 335 | T335A | 43 |
| 337 | L337A | 38 |
| 338 | R338A | 0 |
| 339 | S339L | 46 |
| 340 | T340A | 16 |
| 341 | K341E | 3 |
| 341 | K341A | 18 |
| 343 | T343I | 62 |
| 343 | T343A | 58 |
| 346 | N346Q | 101 |
| 346 | N346A | 92 |
| NA | WT | 100 |

TABLE 11
Results from SPR analysis (mAb01-9985)
Results from hot-spot analysis of OA variant of mAb01-9985

| Residue# | FIX variant | Binding (%) |
|---|---|---|
| 301 | K301A | 94 |
| 332 | D332S | 59 |
| 332 | D332A | 59 |
| 333 | R333A | 73 |
| 334 | A334L | 60 |
| 335 | T335A | 64 |
| 337 | L337A | 61 |
| 338 | R338A | 8 |
| 339 | S339L | 66 |
| 340 | T340A | 29 |
| 341 | K341E | 10 |
| 341 | K341A | 30 |
| 343 | T343I | 84 |
| 343 | T343A | 86 |
| 346 | N346Q | 93 |
| 346 | N346A | 100 |
| NA | WT | 100 |

Hot-Spot Residues for mAb01-9994 and mAb01-9985

Hot-spot residues for mAb01-9994 and mAb01-9985 are defined as positions were substitution of the wild-type residue with alanine reduces the binding of the antibody to 30% or less relative to binding of the antibody to wild-type FIX.

Hot-spot residues for mAb01-9994:

R338, T340 and K341

Hot-spot residues for mAb01-9985:

R338, T340 and K341

For both mAb01-9994 and mAb01-9985 the residue contributing most to binding is R338; substitution of R338 with alanine (R338A) in FIX exhibited the largest impact on antibody binding, which was greatly reduced to no observable binding and 8% for mAb01-9994 and mAb01-9985, respectively, relative to antibody binding to wild-type FIX.

### Example 11: Identification of Hot-Spot Residues on FX

The data provided in the present example determines the hot-spot epitope residues on FX for mAb01-8174. The FX variants used were single-site alanine variants (except for position 118, which is alanine in the wild-type, where an alanine to serine substitution was introduced) of desGla-desEGF1-FX, corresponding to residues 86-448 of SEQ ID NO:2, with a N-terminal His-tag (HHHHHH) (SEQ ID NO:1200), for affinity purification) attached via a short GS-linker (GGGGSGGGGS) (SEQ ID NO:1201). The FX variants tested are listed in Table 12.

TABLE 12
List of generated desGla-desEGF1-FX mutants

| Position¹⁾ | Variant | Domain²⁾ |
|---|---|---|
| 101 | H101A | EGF2 |
| 103 | E103A | EGF2 |
| 113 | R113A | EGF2 |
| 116 | T116A | EGF2 |
| 118 | A118S | EGF2 |
| 127 | T127A | EGF2 |
| 227 | S227A | PD |
| 228 | E228A | PD |
| 229 | F229A | PD |
| 230 | Y230A | PD |
| 266 | E266A | PD |
| 287 | R287A | PD |
| 305 | E305A | PD |
| 419 | L419A | PD |
| 420 | K420A | PD |
| 423 | D423A | PD |
| 424 | R424A | PD |
| 427 | K427A | PD |
| 428 | T428A | PD |

¹⁾According to SEQ ID NO: 2
²⁾EGF2 and PD refer to second epidermal growth factor-like and protease domains, respectively

The wild-type desGla-desEGF1-FX and variants listed in Table 12 were expressed in the HEK293 system and purified via affinity chromatography. Identification of hot-spot epitope residues was done using a Biacore 4000 instrument at 25° C. The wild-type desGla-desEGF1-FX and variants were immobilized on a Series-S Sensor Chip CM5 (GE Healthcare, Catalogue #BR100530) using standard amine coupling chemistry. 10 μM (with 2 fold serial dilutions) of the one-armed variant of mAb01-8174 was injected at the flow rate of 10 μL/min for 300 sec to allow for binding to the immobilized wild-type desGla-desEGF1-FX and variants followed by a 600 sec buffer injection to allow for dissociation of the monovalent antibody. The running buffer (also used for diluting the monovalent antibody and desGLA-desEGF1-hFX variants) contained 10 mM HEPES, 150 mM NaCl, 1 mg/mL BSA and 5 mM CaCl₂) (pH 7.4). No regeneration buffer was used because near complete dissociation of the antibody from the sensor chip was observed during the 600 sec dissociation stage. Binding data were analyzed according to the 1:1 model and the affinity (i.e. dissociation equilibrium constant) for each binding was derived from steady-state fitting of the binding curves in the Biacore 4000 Evaluation Software 1.0 supplied by GE Healthcare. Binding data are reported as fold of affinity of all the FX variants for the antibody with reference to that of the wild-type FX. Relative affinities measured for the FX variants are shown in table 13.

TABLE 13
Relative affinities (K_{D}(variant)/K_{D}(wild-type)) for
binding of one-armed mAb01-8174 to FX variants

| Variant | Relative affinity |
|---|---|
| Wild Type | 1.0 |
| H101A | 3.6 |
| E103A | 3.5 |
| R113A | 2.3 |
| T116A | 0.55 |
| A118S | 1.5 |
| T127A | 0.85 |
| S227A | 2.8 |
| E228A | 0.85 |
| F229A | 0.95 |
| Y230A | No Binding |
| E266A | 0.65 |
| R287A | >5 |
| E305A | 2.1 |
| L419A | 4.5 |
| K420A | >5 |
| D423A | No Binding |
| R424A | No Binding |
| K427A | No Binding |
| T428A | 1.0 |

“No binding” means that binding was too weak to be detected

Hot-Spot Residues for mAb01-8174

As shown in Table 13 alanine-substitutions at positions 230, 423, 424 and 427 in FX completely abrogated binding to one-armed mAb01-8174. Thus, hot-spot residues on FX for binding to mAb01-8174 are:

Y230, D423, R424 and K427

### Example 12: Activity of Monovalent Anti-FIX/FIXa Antibodies in a FXa Generation Assay

To avoid any potential avidity effects arising as a consequence of the bivalency of the conventional IgG antibody format, the stimulatory activity of anti-FIX(a) antibodies on FIXa enzymatic activity towards FX was determined following reformatting into a monovalent one-armed (OA) antibody format (see Example 5). Tested antibodies are listed in Table 14 below. The monovalent OA versions of the anti-FIXa antibodies 224F3 and ACE910 were included for comparison.

The stimulatory activity of OA antibodies was measured in assay buffer (50 mM HEPES, 100 mM NaCl, 5 mM CaCl₂), 0.1% (w/v) PEG8000, pH 7.3+1 mg/ml BSA) at fixed concentrations of phosphatidyl serine (PS):phosphatidyl choline (PC) phospholipid vesicles (final concentration of 500 μM; Haematologic Technologies Inc, USA) and plasma-derived FIXa (final concentrations of 0.04, 0.1, 0.17, 0.2, 0.3, 0.5 or 1 nM; Haematologic Technologies Inc, USA). The concentration of FIXa was chosen to ensure that less than 15% of the substrate FX was converted into FXa. Following pre-incubation in the presence of monovalent OA antibody (final concentrations listed in Table 14), 100 nM plasma-derived FX (Haematologic Technologies Inc, USA) was added to give a final reaction volume of 50 μl, and activation was allowed to proceed for 20 min at room temperature. The reaction was then quenched by addition of 25 μl quench buffer (50 mM HEPES, 100 mM NaCl, 60 mM EDTA, 0.1% PEG8000, pH 7.3+1 mg/ml BSA) and the amount of FXa generated was determined by further addition of 25 μl 2 mM S-2765 chromogenic substrate (Chromogenix, Sweden) and measurement of chromogenic substrate conversion by absorbance measurement at 405 nm (ΔOD/min) in a microplate reader. The measured activity was corrected for background activity by subtraction of the signal measured in the same assay but with FIXa and antibody replaced by assay buffer, and then normalized according to the concentration of FIXa present in the assay ([FIXa]ₜₒₜₐₗ). Dividing this number by the similarly normalized rate of FXa generation in the absence of antibody (A_{FIXa,norm}), an antibody stimulation index was calculated providing the fold stimulation of FIXa activity by the antibody at the concentration used. Due to slow rate of FXa generation by free FIXa, activation reactions in the absence of antibody were carried out as described above but with 5, 10, or 20 nM FIXa present. Measured activities were then background subtracted and normalized according to the FIXa concentration in the assay. For the calculation of the stimulation index, the average of the three normalized activities of free FIXa was used.

Determination of Stimulation Index

In summary, calculation of the stimulation index can be described as follows

Stimulation index=((A_{FIXa+OA}−A_{bckg})/[FIXa]ₜₒₜₐₗ)/A_{FIXa,norm}

where A_{FIXa+OA} is the activity measured in the presence of OA antibody, A_{bckg} is the background activity measured in the absence of FIXa and OA antibody, tot [FIXa]ₜₒₜₐₗ is the FIXa concentration in the assay, and A_{FIXa,norm} is average normalized activity of FIXa.

Determination of FIXa Saturation

The fraction of FIXa saturated with OA antibody in the assay is determined by the concentrations of FIXa and OA antibody, and the equilibrium dissociation constant (K_{d}) governing their interaction. The latter can be measured by techniques known in the art, such as isothermal titration calorimetry (ITC).

Since the stimulation index will increase as the concentration of OA antibody is increased until saturation of FIXa is reached, the concentration of OA antibody in the assay should be chosen to ensure at least 80% saturation of FIXa in the assay to provide a proper determination of the stimulation index at full FIXa saturation.

The fraction of FIXa bound to OA antibody at equilibrium (f_{FIXa+OA}), can be calculated from the total concentrations of FIXa ([FIXa]ₜₒₜₐₗ) and OA antibody ([OA]ₜₒₜₐₗ) in the assay and the equilibrium dissociation constant (K_{d}) for their interaction using the quadractic binding equation as described by Krishnaswamy et al. (1992) J. Biol. Chem., 267:23696-23706 and detailed in Eq. 1 and 2 below, wherein
[FIXa+OA]_{assay} represents the calculated concentration of FIXa-OA antibody complex at equilibrium in the assay
f_{FIXa+OA} represents the calculated fraction (in percent) of FIXa, which is bound to OA antibody at equilibrium in the assay

$${[ FIXa + O A ]}_{assay} = \frac{\begin{matrix}( {[ FIXa ]}_{total} + {[ O A ]}_{total} + {K}_{d} ) - \\ \sqrt{{( {[ FIXa ]}_{total} + {[ O A ]}_{total} + {K}_{d} )}^{2} - 4 \times {[ FIXa ]}_{total} \times {[ O A ]}_{total}}\end{matrix}}{2} \qquad Eq . 1$$

$${f}_{FIXa + O A} = 100 \% \times \frac{{[ FIXa + O A ]}_{assay}}{{[ FIXa ]}_{total}} \qquad Eq . 2$$

The stimulation index for each OA antibody is provided in Table 14. With a concentration of OA 224F3 antibody of 3260 nM in the assay and a K_{d} for the interaction with FIXa of 0.477 nM as reported by Kerschbaumer et al. (U.S. Pat. No. 7,297,336B2), more than 95% of FIXa was bound to the OA 224F3 antibody in the assay. In the case of the OA ACE910 antibody, FIXa stimulation was determined at eight different antibody concentrations which allowed for the estimation of the stimulation index at full FIXa saturation using the quadratic binding equation as outlined above. This also provided an estimated equilibrium dissociation constant (K_{d}) for the interaction of ACE910 with FIXa of 1.1 μM, which is in good agreement with the value of 1.52 μM reported by Kitazawa et al. (2017) Thromb Haemost, 117:1348-1357 and the value of 1.97 μM determined by ITC in Example 13. For the remaining antibodies, the degree of FIXa saturation was not known and the listed stimulation indices therefore represent conservative estimates of the stimulation that would be obtained at a degree of FIXa saturation of 80% or greater. For the tested antibodies the measured stimulation index was found to be higher than that measured for the OA 224F3 and OA ACE910 antibodies.

The anti-FIX mAb ID refers to the ID of the antibody used for reformatting into the OA format. Columns labelled ‘OA antibody concentration (nM)’ and ‘Stimulation index’ list the concentration of OA antibody (nM) used in the assay and the corresponding stimulation of FIXa activity measured relative to free FIXa. In the case of ACE910, the estimated stimulation index at full FIXa saturation is provided.

TABLE 14
Stimulation of FIXa activity by monovalent
one-armed (OA) anti-FIXa antibodies

| anti-FIX mAb ID | OA antibody concentration (nM) | Stimulation Index |
|---|---|---|
| ACE910 | saturation | 1372 |
| 224F3 | 3260 | <10 |
| 01-9016 | 1600 | 5905 |
| 01-9373 | 1600 | 1392 |
| 01-9696 | 1600 | 5166 |
| 01-9697 | 3200 | 6539 |
| 01-9933 | 3600 | 6444 |
| 01-9978 | 3600 | 3667 |
| 01-9985 | 2400 | 13730 |
| 01-9986 | 800 | 5822 |
| 01-9994 | 3600 | 10593 |
| 11-0047 | 800 | 2856 |
| 11-0049 | 800 | 1509 |
| 11-0107 | 800 | 3264 |
| 11-0149 | 800 | 3315 |
| 11-0160 | 800 | 4549 |
| 11-0164 | 800 | 3269 |
| 11-0173 | 1600 | 8480 |
| 11-0174 | 800 | 3690 |
| 11-0723 | 800 | 3772 |
| 11-0923 | 800 | 2823 |
| 11-0929 | 800 | 1559 |
| 11-0931 | 800 | 1882 |
| 11-0932 | 800 | 1688 |
| 11-0933 | 800 | 1614 |
| 11-0935 | 800 | 1572 |
| 11-0937 | 800 | 2684 |
| 11-0938 | 800 | 1818 |
| 11-0939 | 800 | 2011 |
| 11-0962 | 800 | 3372 |
| 11-0965 | 800 | 2857 |
| 11-1021 | 800 | 4646 |
| 11-1024 | 800 | 3654 |
| 11-1030 | 800 | 5751 |
| 11-1033 | 800 | 5126 |
| 11-1039 | 800 | 3960 |
| 11-1042 | 800 | 4316 |
| 11-1073 | 800 | 6146 |
| 11-1076 | 800 | 3587 |
| 11-1091 | 800 | 5402 |
| 11-1094 | 800 | 4083 |
| 11-1204 | 1600 | 9099 |
| 11-1233 | 800 | 1700 |
| 11-1254 | 760 | 1890 |
| 11-1259 | 800 | 2005 |
| 11-1260 | 800 | 1757 |
| 11-1262 | 800 | 1952 |
| 11-1266 | 800 | 1696 |
| 11-1268 | 800 | 1796 |
| 11-1273 | 800 | 1540 |
| 11-1275 | 800 | 1835 |
| 11-1276 | 800 | 1739 |
| 11-1278 | 800 | 1694 |
| 11-1279 | 800 | 1562 |
| 11-1282 | 800 | 1461 |
| 11-1286 | 800 | 1816 |
| 11-1288 | 800 | 2534 |
| 11-1344 | 800 | 4662 |
| 11-1358 | 800 | 2831 |
| 11-1360 | 800 | 1114 |
| 11-1389 | 800 | 5786 |
| 11-1495 | 1600 | 7694 |
| 11-1499 | 1600 | 9013 |
| 11-1501 | 1600 | 10277 |
| 11-1502 | 1600 | 5853 |

### Example 13: Binding Affinities Determined by Isothermal Titration Calorimetry (ITC)

Binding affinities for anti-FIX(a) and anti-FX(a) antibodies were measured by isothermal titration calorimetry (ITC) by using a PEAQ-ITC calorimeter (Malvern, UK). The experiments were conducted at 37° C. and pH 7.4 using 25 mM Tris, 150 mM NaCl, 5 mM CaCl₂ (Tris-buffer). The sample cell (200 μl) contained either FIX, FIXa, or FX (macromolecule) and anti-FIX(a) and anti-FX(a) antibodies (ligand) were injected via a syringe (40 μl). All proteins were extensively dialyzed in Tris-buffer prior to measurements to secure matched buffer conditions. A thermal equilibration step was followed by a 60 s delay and subsequently an initial 0.2 μl injection of antibody, followed by 12-16 injections of 1.5-3 μl of antibody at an interval of 120 s. The stirring speed was maintained at 750 rpm, and the reference power is kept constant at 5-10 μcal/s. The heat associated with each injection of antibody is integrated and plotted against the molar ratio of ligand to macromolecule. The resulting isotherm is fitted to a one-site binding model to obtain the affinity (K_{D}), stoichiometry (n), and enthalpy of interaction (ΔH) using the software provided by the manufacturer. Experiments were performed in at least duplicates. K_{D} values in μM units are reported in Table 15.

TABLE 15
Dissociation constant (K_{D}) values using ITC

| | K_{D}, μM | | |
|---|---|---|---|
| | FIX | FIXa | FX |
| mAb01-9994 | 2.48 ± 0.23 | 1.8 ± 0.13 | NA |
| mAb01-9933 | 2.39 ± 0.33 | 1.3 ± 0.12 | NA |
| mAb01-9985 | 1.92 ± 0.18 | 1.26 ± 0.08 | NA |
| mAb01-9978 | 5.6 ± 0.93 | 3.57 ± 0.52 | NA |
| mAb01-8913 | NA | NA | 0.35 ± 0.19 |
| mAb01-8174 | NA | NA | 0.75 ± 0.66 |
| mAb01-9772 | NA | NA | 1.37 ± 0.39 |
| ACE910 | 2.77 ± 1.44 | 1.97 ± 0.42 | 2.42 ± 0.2 |

NA: Not applicable since antibody is directed to different macromolecule

### Example 14: Activity of Anti-FIX(a)/FX(a) Bispecific Antibodies in a FXa Generation Assay

The procoagulant activity of anti-FIXa/FX bispecific antibodies was determined based on their ability to promote FX activation by FIXa in the presence of a procoagulant phospholipid membrane. The bispecific antibodies (BiAb) tested are listed in Table 16 and ACE910 was included for comparison.

The procoagulant activity of each bispecific antibody is reported as fold stimulation relative to FX activation by free FIXa at a given antibody concentration. Bispecific antibodies were tested at 8 concentrations (made by serial three fold dilutions in assay buffer) by pre-incubation with 35 or 125 μM human plasma-derived FIXa (Haematologic Technologies Inc, USA) and 500 μM 25:75 phosphatidyl serine:phosphatidyl choline phospholipid vesicles (Haematologic Technologies Inc, USA) in assay buffer (50 mM HEPES, 100 mM NaCl, 5 mM CaCl₂), 0.1% (w/v) PEG8000, pH 7.3+1 mg/ml BSA) for 10 min. Activation was then initiated by addition of human plasma-derived FX (Haematologic Technologies Inc, USA) to a concentration of 25 nM. Following 15 min activation at room temperature, the reaction (50 μl) was quenched by addition of 25 μl quench buffer (50 mM HEPES, 100 mM NaCl, 60 mM EDTA, 0.1% PEG8000, pH 7.3+1 mg/ml BSA). The amount of FXa generated was determined by addition of 25 μl 2 mM S-2765 chromogenic substrate (Chromogenix, Sweden) and measurement of chromogenic substrate conversion by absorbance measurement at 405 nm (DOD/min) in a microplate reader. Similarly, FX activation by free FIXa was determined at a FIXa concentration of 25 nM and a reaction time of 60 min.

The measured activity was normalized according to the concentration of FIXa present in the assay and the reaction time. By dividing this number by the similarly normalized rate of FXa generation in the absence of antibody, fold stimulation by the antibody at a given concentration was calculated.

In summary, calculation of biAb stimulation can be described as follows

BiAb stimulation=(A_{FIXa+biAb}/([FIXa]_{assay}×t_{reaction}))/A_{FIXa,norm}

where A_{FIXa+biAb} is the activity measured in the presence of bispecific antibody, [FIXa]_{assay} is the FIXa concentration in the assay, t_{reaction} is the reaction time, and A_{FIXa,norm} is the normalized activity of free FIXa.

Table 16 lists the maximum stimulation determined for each bispecific antibody among the 8 antibody concentrations tested as well as the concentration at which maximum stimulation (fold) was observed. For all tested bispecific antibodies the maximum stimulation was found to be higher than that measured for ACE910, which was tested at a concentration interval from 0 to 15300 nM.

TABLE 16
Maximum stimulation by bispecific anti-FIXa/FX antibodies

| | | BiAb conc at | |
|---|---|---|---|
| BiAb antibody | Concentration | maximum | Maximum |
| ID | span tested (nM) | stimulation (nM) | stimulation (fold) |
| ACE910 | 14.38-31440 | 10480 | 891 |
| bimAb05-0745 | 3.65-7980 | 7980 | 21143 |
| bimAb05-3761 | 3.69-8070 | 2690 | 16332 |
| bimAb05-3769 | 3.735-8170 | 8170 | 27417 |
| bimAb05-0746 | 5.1-11150 | 3717 | 14595 |
| bimAb05-2112 | 3.575-7820 | 7820 | 30426 |
| bimAb05-2113 | 3.475-7600 | 7600 | 26321 |
| bimAb05-2114 | 3.465-7580 | 7580 | 35222 |
| bimAb05-2115 | 3.56-7790 | 7790 | 14921 |
| bimAb05-3755 | 1.93-4212 | 156 | 7573 |

### Example 15: TGT of Anti-FIX(a)/FX(a) Bispecific Antibodies in Haemophilia a (HA) Plasma

Thrombin generation tests (TGT) were conducted in an automated HTP 384-well setup triggering with tissue factor. In brief, 10 μl antibodies were added to 30 μl haemophilia A (HA) plasma (George King). Then, 10 μl TissueFactor trigger (Thrombinoscope, #TS31.00) mixed with phospholipids was added, followed by addition of 10 μl thrombin substrate (FluCa, Thrombinoscope, #TS50.00) to a final assay volume of 60 μl. Fluorescence time series were measured at room temperature on a Perkin Elmer EnVision multi-label plate reader at 1-minute intervals for 2 hours. Thrombograms were calculated as the smoothed first derivative of the fluorescence time series. Peak height was calculated as the maximum value observed in the thrombogram, and normalized (peak ratio) to the peak height observed for the ACE910 reference at the highest concentration (333 nM). The test results for the bispecific antibodies (bimAb) are shown in Table 17. Peak ratio (fold) is the maximum value observed for the bimAb in the thrombogram relative to the value for ACE910 at 333 nM. The concentration (nM) at which the peak is observed is also listed.

TABLE 17
TGT activity of anti-FIX/FX bimAbs relative to ACE910

| | anti-FIX | anti-FX | Concentration | Peak ratio |
|---|---|---|---|---|
| bimAb ID | mAb ID | mAb ID | (nM) | (fold) |
| bimAb05-0396 | mAb11-1495 | mAb01-8174 | 111 | 1.18 |
| bimAb05-0417 | mAb11-1501 | mAb01-8174 | 111 | 1.25 |
| bimAb05-0438 | mAb11-1502 | mAb01-8174 | 333 | 1.10 |
| bimAb05-0745 | mAb01-9994 | mAb01-8174 | 111 | 1.24 |
| bimAb05-2114 | mAb01-9985 | mAb01-9772 | 333 | 1.07 |
| bimAb05-2375 | mAb01-9016 | mAb01-8174 | 111 | 1.14 |
| bimAb05-2379 | mAb01-9016 | mAb01-8913 | 666 | 1.26 |
| bimAb05-2532 | mAb01-9373 | mAb01-6723 | 333 | 1.24 |
| bimAb05-3416 | mAb01-9985 | mAb01-6723 | 111 | 1.13 |
| bimAb05-3769 | mAb01-9985 | mAb01-8174 | 111 | 1.27 |
| bimAb05-3770 | mAb01-9986 | mAb01-8174 | 333 | 1.23 |
| bimAb05-3862 | mAb01-9696 | mAb01-8174 | 333 | 1.52 |
| bimAb05-3880 | mAb11-0047 | mAb01-8174 | 333 | 1.4 |
| bimAb05-3886 | mAb11-0049 | mAb01-8174 | 333 | 1.16 |
| bimAb05-3955 | mAb11-0107 | mAb01-8174 | 333 | 1.34 |
| bimAb05-4100 | mAb11-0149 | mAb01-8174 | 333 | 1.25 |
| bimAb05-4114 | mAb11-0160 | mAb01-8174 | 333 | 1.26 |
| bimAb05-4121 | mAb11-0164 | mAb01-8174 | 333 | 1.34 |
| bimAb05-4220 | mAb11-0962 | mAb01-8174 | 333 | 1.39 |
| bimAb05-4226 | mAb11-0965 | mAb01-8174 | 333 | 1.31 |
| bimAb05-4271 | mAb11-0173 | mAb01-8174 | 111 | 1.32 |
| bimAb05-4283 | mAb11-1021 | mAb01-8174 | 333 | 1.19 |
| bimAb05-4289 | mAb11-1024 | mAb01-8174 | 333 | 1.06 |
| bimAb05-4292 | mAb11-1033 | mAb01-8174 | 111 | 1.19 |
| bimAb05-4293 | mAb11-1030 | mAb01-8174 | 111 | 1.35 |
| bimAb05-4387 | mAb11-1039 | mAb01-8174 | 333 | 1.28 |
| bimAb05-4392 | mAb11-1042 | mAb01-8174 | 111 | 1.24 |
| bimAb05-4419 | mAb11-0174 | mAb01-8174 | 111 | 1.21 |
| bimAb05-4422 | mAb11-1073 | mAb01-8174 | 333 | 1.2 |
| bimAb05-4428 | mAb11-1076 | mAb01-8174 | 111 | 1.12 |
| bimAb05-4443 | mAb11-1094 | mAb01-8174 | 333 | 1.14 |
| bimAb05-4444 | mAb11-1091 | mAb01-8174 | 333 | 1.19 |
| bimAb05-4601 | mAb11-0937 | mAb01-8174 | 111 | 1.17 |
| bimAb05-4604 | mAb11-0923 | mAb01-8174 | 333 | 1.02 |
| bimAb05-4611 | mAb11-0935 | mAb01-8174 | 333 | 1.02 |
| bimAb05-4612 | mAb11-0938 | mAb01-8174 | 111 | 1.23 |
| bimAb05-4613 | mAb11-0933 | mAb01-8174 | 333 | 1.03 |
| bimAb05-4684 | mAb01-9985 | mAb11-1114 | 333 | 1.05 |
| bimAb05-4685 | mAb01-9985 | mAb11-1115 | 111 | 1.12 |
| bimAb05-4686 | mAb01-9985 | mAb11-1116 | 333 | 1.16 |
| bimAb05-4687 | mAb01-9985 | mAb11-1117 | 333 | 1.12 |
| bimAb05-4688 | mAb01-9985 | mAb11-1118 | 111 | 1.16 |
| bimAb05-4689 | mAb01-9985 | mAb11-1119 | 333 | 1.26 |
| bimAb05-4690 | mAb01-9985 | mAb11-1121 | 333 | 1.36 |
| bimAb05-4693 | mAb01-9985 | mAb11-1123 | 333 | 1.02 |
| bimAb05-4696 | mAb01-9985 | mAb11-1125 | 333 | 1.3 |
| bimAb05-4704 | mAb01-9985 | mAb11-1416 | 333 | 1.17 |
| bimAb05-4708 | mAb01-9985 | mAb11-1420 | 111 | 1.02 |
| bimAb05-4710 | mAb01-9985 | mAb11-1422 | 333 | 1.1 |
| bimAb05-4756 | mAb11-1204 | mAb01-8174 | 111 | 1.20 |
| bimAb05-4788 | mAb11-1233 | mAb01-8174 | 333 | 1.13 |
| bimAb05-4884 | mAb11-1254 | mAb01-8174 | 316 | 1.16 |
| bimAb05-4895 | mAb11-1262 | mAb01-8174 | 111 | 1.28 |
| bimAb05-4896 | mAb11-1259 | mAb01-8174 | 333 | 1.13 |
| bimAb05-4898 | mAb11-1260 | mAb01-8174 | 333 | 1.17 |
| bimAb05-4903 | mAb11-1268 | mAb01-8174 | 333 | 1.1 |
| bimAb05-4906 | mAb11-1266 | mAb01-8174 | 111 | 1.38 |
| bimAb05-4910 | mAb11-1273 | mAb01-8174 | 333 | 1.12 |
| bimAb05-4914 | mAb11-1275 | mAb01-8174 | 333 | 1.16 |
| bimAb05-4915 | mAb11-1276 | mAb01-8174 | 111 | 1.23 |
| bimAb05-4919 | mAb11-1278 | mAb01-8174 | 333 | 1.17 |
| bimAb05-4920 | mAb11-1282 | mAb01-8174 | 111 | 1.22 |
| bimAb05-4921 | mAb11-1279 | mAb01-8174 | 333 | 1.14 |
| bimAb05-4924 | mAb11-1288 | mAb01-8174 | 333 | 1.22 |
| bimAb05-4927 | mAb11-1286 | mAb01-8174 | 333 | 1.22 |
| bimAb05-5092 | mAb11-1344 | mAb01-8174 | 111 | 1.23 |
| bimAb05-5095 | mAb11-0723 | mAb01-8174 | 111 | 1.31 |
| bimAb05-5204 | mAb11-1358 | mAb01-8174 | 333 | 1.5 |
| bimAb05-5205 | mAb11-1360 | mAb01-8174 | 111 | 1.38 |
| bimAb05-5240 | mAb11-1389 | mAb01-8174 | 111 | 1.29 |
| bimAb05-5340 | mAb01-9985 | mAb11-1431 | 111 | 1.28 |
| bimAb05-5341 | mAb01-9985 | mAb11-1441 | 111 | 1.05 |
| bimAb05-5342 | mAb01-9985 | mAb11-1439 | 333 | 1.14 |
| bimAb05-5344 | mAb01-9985 | mAb11-1443 | 333 | 1.03 |
| bimAb05-5346 | mAb01-9985 | mAb11-1444 | 158 | 1.17 |
| bimAb05-5348 | mAb01-9985 | mAb11-1445 | 111 | 1.19 |
| bimAb05-5353 | mAb01-9985 | mAb11-1453 | 333 | 1.15 |
| bimAb05-5354 | mAb01-9985 | mAb11-1451 | 116 | 1.01 |
| bimAb05-5356 | mAb01-9985 | mAb11-1452 | 111 | 1.16 |
| bimAb05-5357 | mAb01-9985 | mAb11-1455 | 333 | 1.11 |
| bimAb05-5358 | mAb01-9985 | mAb11-1433 | 111 | 1.22 |
| bimAb05-5359 | mAb01-9985 | mAb11-1456 | 333 | 1.07 |
| bimAb05-5361 | mAb01-9985 | mAb11-1459 | 111 | 1.21 |
| bimAb05-5362 | mAb01-9985 | mAb11-1457 | 111 | 1.39 |
| bimAb05-5363 | mAb01-9985 | mAb11-1434 | 333 | 1.56 |
| bimAb05-5364 | mAb01-9985 | mAb11-1458 | 111 | 1.18 |
| bimAb05-5365 | mAb01-9985 | mAb11-1460 | 333 | 1.04 |
| bimAb05-5366 | mAb01-9985 | mAb11-1461 | 333 | 1.12 |
| bimAb05-5367 | mAb01-9985 | mAb11-1465 | 333 | 1.15 |
| bimAb05-5369 | mAb01-9985 | mAb11-1466 | 333 | 1.17 |
| bimAb05-5370 | mAb01-9985 | mAb11-1463 | 333 | 1.06 |
| bimAb05-5371 | mAb01-9985 | mAb11-1435 | 208 | 1.19 |
| bimAb05-5372 | mAb01-9985 | mAb11-1464 | 333 | 1.05 |
| bimAb05-5373 | mAb01-9985 | mAb11-1467 | 333 | 1.15 |
| bimAb05-5374 | mAb01-9985 | mAb11-1468 | 333 | 1.07 |
| bimAb05-5375 | mAb01-9985 | mAb11-1472 | 308 | 1.27 |
| bimAb05-5377 | mAb01-9985 | mAb11-1473 | 111 | 1.27 |
| bimAb05-5378 | mAb01-9985 | mAb11-1470 | 333 | 1.16 |
| bimAb05-5379 | mAb01-9985 | mAb11-1436 | 111 | 1.07 |
| bimAb05-5380 | mAb01-9985 | mAb11-1471 | 333 | 1.09 |
| bimAb05-5381 | mAb01-9985 | mAb11-1474 | 333 | 1.15 |
| bimAb05-5383 | mAb01-9985 | mAb11-1477 | 111 | 1.05 |
| bimAb05-5385 | mAb01-9985 | mAb11-1478 | 333 | 1.03 |
| bimAb05-5386 | mAb01-9985 | mAb11-1476 | 333 | 1.08 |
| bimAb05-5387 | mAb01-9985 | mAb11-1479 | 333 | 1.27 |
| bimAb05-5388 | mAb01-9985 | mAb11-1480 | 111 | 1.36 |
| bimAb05-5389 | mAb01-9985 | mAb11-1483 | 111 | 1.19 |
| bimAb05-5390 | mAb01-9985 | mAb11-1437 | 133 | 1.06 |
| bimAb05-5396 | mAb01-9985 | mAb11-1487 | 111 | 1.07 |
| bimAb05-5406 | mAb01-9985 | mAb11-1494 | 333 | 1.19 |

### Example 16: Activity of Bispecific Anti-FIX(a)/FX(a) Antibodies in a Thrombin Generation Test (TGT) in Human Haemophilia a Platelet-Poor and Platelet-Rich Mimic Plasma

The procoagulant activity of the bispecific antibodies bimAb05-0745, bimAb05-3761, bimAb05-3769, bimAb05-0746, bimAb05-2112, bimAb05-2113 and bimAb005-2114 was determined based on their ability to promote thrombin generation in the presence of either a procoagulant synthetic phospholipid membrane or platelets according to the principles described by Hemker et al. (Pathophysiol Haemost Thromb, 2002; 32:249-253). ACE910 was included for comparison. Each antibody (test compound) was tested in a thrombin generation test (TGT) using commercially available Haemophilia A (HA) patient pooled platelet-poor plasma (HA-PPP) and HA-induced human platelet-rich plasma (HA-PRP) freshly prepared from healthy consenting donors.

Materials and Methods:

Preparation of Haemophilia A-Induced Human Platelet-Rich Plasma (HA-PRP)

Blood was obtained from healthy consenting donors by venipuncture. Six volumes of blood was collected into 1 volume acid citrate dextrose (ACD; 85 mM sodium citrate, 110 mM dextrose, and 62.3 mM citric acid, pH 4.9), final pH 6.5, and centrifuged for 20 min at 220 g at room temperature (RT). Platelet-rich plasma (PRP) was collected and platelet concentrations were determined with a Medonic CA 620 hematology analyzer (Boule Diagnostics AB, Spånga, Sweden).

The red blood cell-containing plasma part was centrifuged for another 10 min at 600 g at RT. Platelet-poor plasma (PPP) was collected and used to dilute the PRP to ˜300,000 platelets/μl. HA conditions were induced by addition of a FVIII-neutralising anti-human FVIII antibody (Sheep anti-Human Factor VIII—5 mg, Haematologic Technologies, VT, USA) to a final concentration of 0.1 mg/ml and rotated gently at 2 rpm for 30 minutes at RT.

Thrombin Generation Test

Thrombin generation tests (TGT) in both HA-PPP (George King Bio-Medical Inc, KS, USA) (Exp. A) and HA-PRP (Exp. B) were performed by standard calibrated automated thrombography using a 96-well plate fluorometer (Fluoroscan Ascent FL, Thermolabsystems, Helsinki, Finland). Reaction mixtures contained 70 μl HA-PRP (˜300,000 platelets/μl) or HA-PPP, 10 μl test compound dilution (diluted in 20 mM HEPES, 140 mM NaCl, pH 7.4, 2% BSA), 20 μl CAT reagents containing tissue factor (TF) (PRP reagent; TF without synthetic phospholipids, PPP-reagent LOW; TF with synthetic phospholipids, 1 pM TF final, Thrombinoscope BV, Maastricht, the Netherlands) or Thrombin Calibrator (Thrombinoscope BV), and 20 μl of a mixture containing the fluorescently labelled thrombin substrate z-Gly-Gly-Arg-AMC (3 mM) and CaCl₂) (90 mM) (Thrombinoscope BV). TGT was performed at up to eight concentrations of test compound (0.3, 1.0, 3, 10, 30, 100, 300, and 900 nM, final plasma concentration) or added buffer (20 mM HEPES, 140 mM NaCl, pH 7.4, 2% BSA) only (representing HA control). The concentration ranges were tested in at least three independent experiments in HA-PPP from the same stock or in blood from four different donors. Normal control levels in TGT were measured using untreated human PRP or CRYOcheck™ pooled normal human PPP plasma (Precision Biologic Inc., Dartmouth, Canada) added buffer (20 mM HEPES, 140 mM NaCl, pH 7.4, 2% BSA) only. The TGT was allowed to proceed for a total of 90 minutes and the TGT parameter Peak Thrombin Height (nM) was analysed by Thrombinoscope software (Thrombinoscope BV).

Results and Discussion

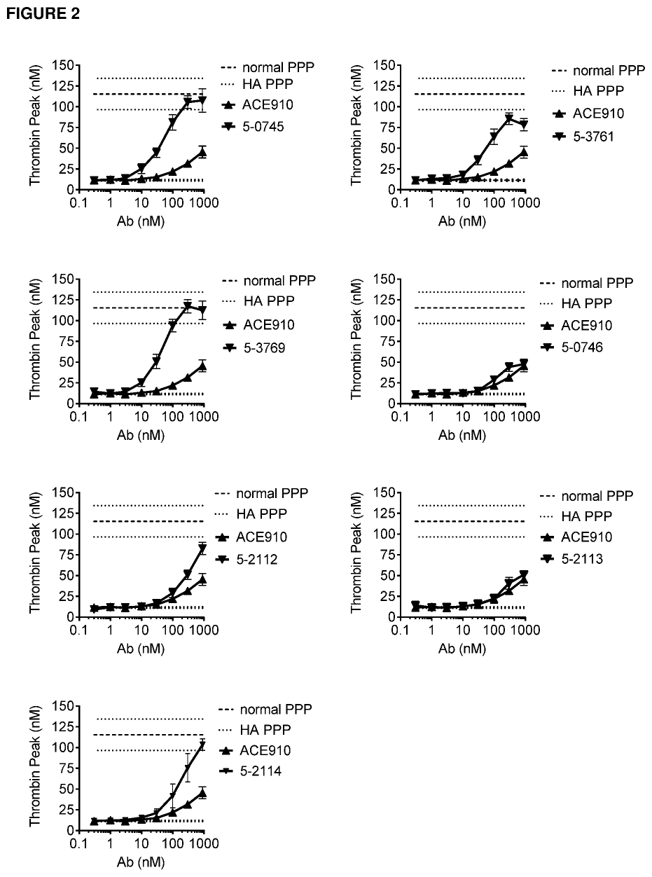

Exp. A: FIG. 2 and Table 18 shows the measured peak thrombin generation rates for each bispecific antibody at the concentrations tested in HA-PPP. The data show that all test compounds increase the peak thrombin formation above the level observed in the absence of antibody, i.e. exhibit procoagulant activity. In addition, thrombin generation levels between 10 and 300 nM for bimAb05-0745, bimAb05-3761, bimAb05-3769, bimAb05-0746, bimAb05-2112, bimAb05-2113, and bimAb05-2114 are higher than that observed for ACE910, demonstrating superior potency. Moreover, thrombin generation levels at 300 to 900 nM of bimAb05-0745, bimAb05-3761, bimAb05-3769, bimAb05-2112, and bimAb05-2114 are higher than that observed with 900 nM ACE910, demonstrating higher potencies and efficacies of these compounds compared to ACE910.

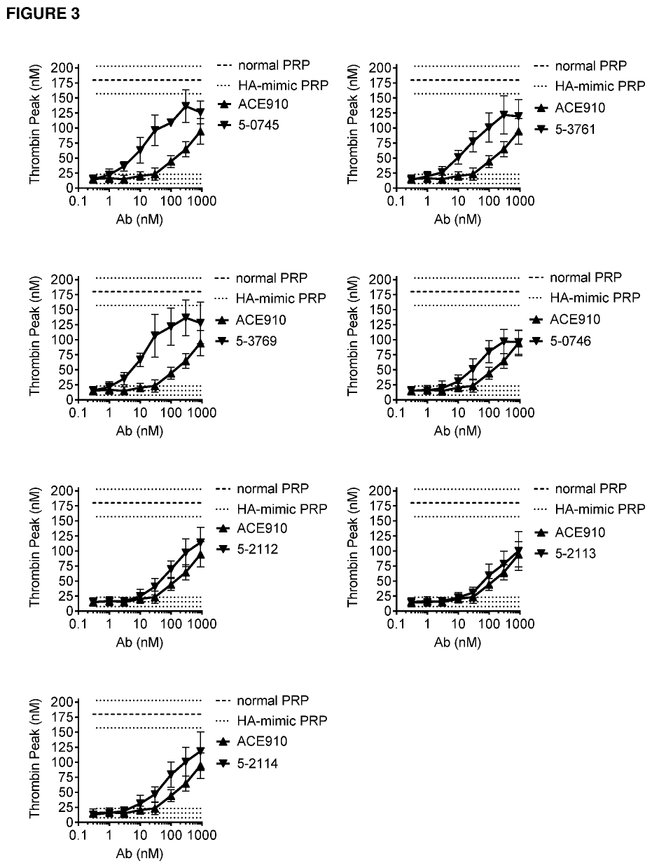

Exp. B: FIG. 3 and Table 19 shows the measured peak thrombin generation for each bispecific antibody at the concentrations tested in HA-PRP. Under these conditions, bimAb05-0745, bimAb05-3761, bimAb05-3769, bimAb05-0746, bimAb05-2112, bimAb05-2113, and bimAb05-2114 also display better potencies and efficacies compared to ACE910.

Thrombin generation test (TGT) of the bispecific antibodies bimAb05-0745, bimAb05-3761, bimAb05-3769, bimAb05-0746, bimAb05-2112, bimAb05-2113, bimAb05-2114, and ACE910 in human tissue factor activated haemophilia A platelet-poor plasma (PPP). Mean peak thrombin generation levels±standard deviation measured at each of the tested compound concentrations in at least three independent experiments in HA-PPP (Exp. A).

TABLE 18
Thrombin generation test (TGT) in HA-PPP (Exp. A)

| Exp. A | Peak thrombin | Peak thrombin | Peak thrombin |
|---|---|---|---|
| Compound | (mean ± SD in nM) | (mean ± SD in nM) | (mean ± SD in nM) |
| concentration (nM) | for ACE910 | for bimAb05-0745 | for bimAb05-3761 |
| 0 | 15.5 ± 7.8 | 11.6 ± 0.9 | 11.6 ± 0.9 |
| 0.3 | 11.5 ± 1.1 | 11.6 ± 1.9 | 11.4 ± 0.7 |
| 1 | 12.6 ± 0.1 | 11.8 ± 1.2 | 13.5 ± 1.6 |
| 3 | 11.2 ± 2.5 | 14.0 ± 3.6 | 13.8 ± 2.7 |
| 10 | 13.3 ± 0.0 | 25.5 ± 5.8 | 18.1 ± 3.8 |
| 30 | 15.3 ± 4.3 | 44.8 ± 5.8 | 35.7 ± 5.4 |
| 100 | 22.0 ± 4.1 | 81.4 ± 9.2 | 64.1 ± 9.2 |
| 300 | 31.6 ± 4.0 | 105.7 ± 7.7 | 85.7 ± 7.0 |
| 900 | 45.6 ± 7.2 | 107.7 ± 14.1 | 78.4 ± 7.5 |
| Exp. A | Peak thrombin | Peak thrombin | Peak thrombin |
| Compound | (mean ± SD in nM) | (mean ± SD in nM) | (mean ± SD in nM) |
| concentration (nM) | for bimAb05-3769 | for bimAb05-0746 | for bimAb05-2112 |
| 0 | 11.6 ± 0.9 | 11.6 ± 0.9 | 11.6 ± 0.9 |
| 0.3 | 14.6 ± 3.8 | 11.1 ± 1.2 | 9.4 ± 2.5 |
| 1 | 12.3 ± 0.8 | 12.4 ± 2.5 | 11.8 ± 1.0 |
| 3 | 14.7 ± 3.2 | 13.1 ± 3.2 | 12.0 ± 1.1 |
| 10 | 25.4 ± 4.8 | 12.6 ± 1.3 | 12.7 ± 1.2 |
| 30 | 50.8 ± 8.7 | 15.3 ± 0.9 | 17.0 ± 4.0 |
| 100 | 94.1 ± 7.5 | 29.0 ± 3.6 | 29.1 ± 5.7 |
| 300 | 117.5 ± 8.1 | 44.2 ± 5.2 | 51.2 ± 5.7 |
| 900 | 112.5 ± 11.0 | 47.8 ± 6.0 | 82.8 ± 7.5 |

| Exp. A | Peak thrombin | Peak thrombin |
|---|---|---|
| Compound | (mean ± SD in nM) | (mean ± SD in nM) |
| concentration (nM) | for bimAb05-2113 | for bimAb05-2114 |
| 0 | 11.6 ± 0.9 | 11.6 ± 0.9 |
| 0.3 | 14.2 ± 5.2 | 11.9 ± 0.5 |
| 1 | 11.5 ± 1.2 | 12.3 ± 0.8 |
| 3 | 11.8 ± 0.7 | 12.9 ± 1.2 |
| 10 | 13.0 ± 2.7 | 15.7 ± 2.8 |
| 30 | 15.7 ± 5.4 | 21.3 ± 5.1 |
| 100 | 22.4 ± 5.5 | 41.9 ± 14.5 |
| 300 | 40.7 ± 7.2 | 75.8 ± 17.0 |
| 900 | 51.1 ± 4.0 | 103.7 ± 7.1 |

Thrombin generation test (TGT) of the bispecific antibodies bimAb05-0745, bimAb05-3761, bimAb05-3769, bimAb05-0746, bimAb05-2112, bimAb05-2113, bimAb05-2114 and ACE910 in human tissue factor activated haemophilia A platelet-rich plasma (PRP). Mean peak thrombin generation±standard deviation at each of the tested compound concentrations from four independent experiments in HA-PRP (Exp. B).

TABLE 19
Thrombin generation test (TGT) in HA-PRP (Exp. B)

| Exp. B | Peak thrombin | Peak thrombin | Peak thrombin |
|---|---|---|---|
| Compound | (mean ± SD in nM) | (mean ± SD in nM) | (mean ± SD in nM) |
| concentration (nM) | for ACE910 | for bimAb05-0745 | for bimAb05-3761 |
| 0 | 15.5 ± 7.8 | 15.5 ± 7.8 | 15.5 ± 7.8 |
| 0.3 | 15.2 ± 7.1 | 12.0 ± 2.1 | 11.6 ± 0.7 |
| 1 | 16.9 ± 7.5 | 11.9 ± 1.4 | 14.0 ± 1.6 |
| 3 | 15.0 ± 6.9 | 14.5 ± 4.2 | 14.3 ± 3.0 |
| 10 | 20.3 ± 7.0 | 23.5 ± 5.2 | 18.8 ± 4.2 |
| 30 | 23.3 ± 10.3 | 45.0 ± 7.1 | 36.1 ± 6.5 |
| 100 | 44.3 ± 10.0 | 79.9 ± 10.6 | 65.8 ± 10.4 |
| 300 | 64.9 ± 12.6 | 106.0 ± 9.4 | 86.0 ± 8.6 |
| 900 | 94.5 ± 21.3 | 108.9 ± 17.0 | 80.8 ± 7.2 |
| Exp. B | Peak thrombin | Peak thrombin | Peak thrombin |
| Compound | (mean ± SD in nM) | (mean ± SD in nM) | (mean ± SD in nM) |
| concentration (nM) | for bimAb05-3769 | for bimAb05-0746 | for bimAb05-2112 |
| 0 | 15.5 ± 7.8 | 15.5 ± 7.8 | 15.5 ± 7.8 |
| 0.3 | 13.0 ± 2.6 | 11.3 ± 1.4 | 9.0 ± 2.9 |
| 1 | 12.6 ± 0.8 | 13.0 ± 2.7 | 12.0 ± 1.2 |
| 3 | 15.7 ± 3.1 | 13.7 ± 3.7 | 12.3 ± 1.1 |
| 10 | 24.8 ± 5.7 | 13.0 ± 1.2 | 12.9 ± 1.4 |
| 30 | 49.8 ± 10.4 | 15.5 ± 1.0 | 17.1 ± 4.8 |
| 100 | 95.6 ± 8.5 | 30.4 ± 2.8 | 30.8 ± 5.8 |
| 300 | 118.6 ± 9.6 | 45.3 ± 5.8 | 51.3 ± 6.9 |
| 900 | 114.4 ± 12.6 | 49.5 ± 5.9 | 83.3 ± 9.1 |

| Exp. B | Peak thrombin | Peak thrombin |
|---|---|---|
| Compound | (mean ± SD in nM) | (mean ± SD in nM) |
| concentration (nM) | for bimAb05-2113 | for bimAb05-2114 |
| 0 | 15.5 ± 7.8 | 15.5 ± 7.8 |
| 0.3 | 11.7 ± 1.1 | 11.9 ± 0.5 |
| 1 | 11.6 ± 1.4 | 12.3 ± 0.8 |
| 3 | 11.7 ± 0.9 | 12.9 ± 1.2 |
| 10 | 13.6 ± 2.9 | 15.7 ± 2.8 |
| 30 | 16.8 ± 6.1 | 21.3 ± 5.1 |
| 100 | 24.2 ± 5.2 | 41.9 ± 14.5 |
| 300 | 43.3 ± 6.2 | 75.8 ± 17.0 |
| 900 | 52.3 ± 4.1 | 103.7 ± 7.1 |

### Example 17: In Vivo Efficacy of Bispecific Anti-FIX(a)/FX(a) Antibodies in a Tain Vein Transection (TVT) Model

In vivo efficacy was determined using a Tail Vein Transection (TVT) model in FVIII knockout-mice. The efficacy of a high dose (8 mg/kg) of antibody test compounds and ACE910 was investigated in a Tail Vein Transection (TVT) study in FVIII knockout mice (B6; 129S-F8tm1Kaz/J, The Jackson Laboratory, Bar Harbor, Me., US) co-treated with human FIX (2 mg/kg) (Benefix, Pfizer, New York City, N.Y., US) and FX (1.5 mg/kg) (Haematologic Technologies, INC, Essex Junction, VT, US). In short, the mice were anaesthetized with isoflurane and placed on a heating pad, set to keep animal body temperature at 37° C., with their tails immersed in saline (37° C.). Dosing was performed in the right lateral tail vein 5 minutes prior to the injury. In the present TVT model (Johansen et al., Haemophilia, 2016, 625-31) the lateral vein was transected. If the bleeding stopped at 10, 20, or 30 min, the tail was taken up from the saline, and wound was gently wiped with a saline wetted gauze swab. Total blood loss was determined after 40 min by quantifying the amount of haemoglobin in the saline (see Table 20). The efficacy of the antibody test compound was compared with a One-way ANOVA followed by a Tukey multiple comparison test. A p-value<0.05 was considered significant.

TABLE 20
Comparison of the efficacy in the
bleeding models in F8 knockout mice

| | Blood loss mean |
|---|---|
| Bispecific antibody ID | (nmol haemoglobin) |
| Vehicle (control) | 4441 |
| bimAb05-0745 | 1072* |
| bimAb05-0746 | 925* |
| bimAb05-3761 | 566* |
| bimAb05-3769 | 860* |
| bimAb05-2112 | 1301* |
| bimAb05-2113 | 450* |
| bimAb05-2114 | 1222* |
| ACE910 | 1462* |

*Significantly different from vehicle treated FVIII knockout mice

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A multispecific antibody or antigen-binding fragment thereof capable of stimulating the enzymatic activity of FIXa towards FX comprising a first antigen-binding site capable of binding to FIX (SEQ ID NO:1) and/or the activated form thereof (FIXa), and a second antigen-binding site capable of binding to FX (SEQ ID NO:2) and/or the activated form thereof (FXa).

2. The antibody or antigen-binding fragment thereof according to claim 1, wherein the antibody or antigen-binding fragment thereof
competes with a reference antibody for binding to FIX(a) wherein the reference antibody comprises
a) a heavy chain variable domain identified by SEQ ID NO:35 and a light chain variable domain identified by SEQ ID NO:39, or
b) a heavy chain variable domain identified by SEQ ID NO:43 and a light chain variable domain identified by SEQ ID NO:47, or
c) a heavy chain variable domain identified by SEQ ID NO:51 and a light chain variable domain identified by SEQ ID NO:55, or
d) a heavy chain variable domain identified by SEQ ID NO:67 and a light chain variable domain identified by SEQ ID NO:71,
and/or
competes with a reference antibody for binding to FX(a) wherein the reference antibody comprises
e) a heavy chain variable domain identified by SEQ ID NO:467 and a light chain variable domain identified by SEQ ID NO:471, or
f) a heavy chain variable domain identified by SEQ ID NO:483 and a light chain variable domain identified by SEQ ID NO:487.

3. The antibody or antigen-binding fragment thereof according to claim 1, wherein the first antigen-binding site is capable of binding an epitope comprising amino acid residues R338, T340 and K341 of FIX(a).

4. The antibody or antigen-binding fragment thereof according to claim 3, wherein the first antigen-binding site is capable of binding an epitope comprising amino acid residues L337, R338, T340 and K341 and optionally T343 of FIX(a).

5. The antibody or antigen-binding fragment thereof according to claim 3, wherein the first antigen-binding site is capable of binding an epitope comprising amino acid residues L337, R338, S339, T340, and K341 of FIX(a).

6. The antibody or antigen-binding fragment thereof according to claim 1, wherein the second antigen-binding site is capable of binding to the EGF-2 domain and/or the C-terminal trypsin-like serine protease domain of FX(a).

7. The antibody or antigen-binding fragment thereof according to claim 1, wherein the first antigen-binding site is capable of binding to an epitope comprising R338, T340, K341 of FIX(a), and wherein the second antigen-binding site is capable of binding an epitope comprising amino acid residues Y230, D423, R424 and K427 of FX(a).

8. The antibody or antigen-binding fragment thereof according to claim 1, wherein the first antigen-binding site is capable of binding to an epitope comprising R338, T340, K341, and optionally T343 of FIX(a), and wherein the second antigen-binding site is capable of binding an epitope comprising amino acid residues
i) E103, Q104, V108, R113, T116, L117, D119, I125, T127, E228, F229, Y230, E266, R287, P291, I292, P304, L419, K420, D423, R424, M426, K427 and T428 of FX(a) or
ii) E103, Q104, V108, R113, T116, L117, A118, D119, I125, T127, S227, E228, Y230, R287, I292, L303, P304, L419, K420, D423, R424, M426, K427 and T428 of FX(a).

9. The antibody or antigen-binding fragment thereof according to claim 1, wherein the first antigen-binding site is capable of binding to an epitope comprising L337, R338, S339, T340 and K341 of FIX(a), and wherein the second antigen-binding site is capable of binding an epitope comprising amino acid residues
i) E103, Q104, V108, R113, T116, L117, D119, I125, T127, E228, F229, Y230, E266, R287, P291, I292, P304, L419, K420, D423, R424, M426, K427 and T428 of FX(a)
or
ii) E103, Q104, V108, R113, T116, L117, A118, D119, I125, T127, S227, E228, Y230, R287, I292, L303, P304, L419, K420, D423, R424, M426, K427 and T428 of FX(a).

10. The antibody according to claim 1, wherein the first antigen-binding site comprises
i. a paratope comprising amino acid residues D30, D31, W53, S102, S104 and N107 in the heavy chain variable domain (SEQ ID NO:35) and amino acid residues Y91 and S92 in the light chain variable domain (SEQ ID NO:39), optionally comprising one or two substitution(s) in the eight recited paratope amino acid residues, or
ii. a paratope comprising amino acid residues H30, D31, W53, D56, S102, S104, Y106 and N107 in the heavy chain variable domain (SEQ ID NO:67) and residues Y91 and S92 in the light chain variable domain (SEQ ID NO:71), optionally comprising one or two substitution(s) in the ten recited paratope amino acid residues, or
iii. a paratope comprising amino acid residues H30, D31, W53, S102, S104, Y106 and N107 in the heavy chain variable domain (SEQ ID NO:51) and residues Y91 and S92 in the light chain variable domain (SEQ ID NO:55), optionally comprising one or two substitution(s) in the nine recited paratope amino acid residues,
and the second antigen-binding site comprises
a. a paratope comprising amino acid residues K23, G24, S25, G26, Y27, W33, D52, S54, D55, Y57, S77, L99, H100, Y101, Y102, N103 and S104 in the variable heavy chain domain (SEQ ID NO:483) and amino acid residues S30, S31, Y33, Y50, Q52, S54, R55, R57, Y92 and D94 in the light chain variable domain (SEQ ID NO:487), optionally comprising one, two, three, four or five substitutions in the 27 recited paratope amino acid residues, or
b. a paratope comprising amino acid residues K23, S25, G26, Y27, F29, W33, D52, S54, D55, F57, S77, H100, Y101, Y102, N103 and S104 in the heavy chain variable domain (SEQ ID NO:467) and residues V29, S30, S31, Y33, Y50, Q52, S54, R55, R57 and D94 in the light chain variable domain (SEQ ID NO:471), optionally comprising one, two, three, four or five substitutions in the 26 recited paratope amino acid residues.

11. The antibody or antigen-binding fragment thereof according to claim 10 wherein said substitutions are conservative substitutions.

12. The antibody or antigen-binding fragment thereof according to claim 1 wherein
the first antigen-binding site is comprised by an anti-FIX(a) antibody or antigen-binding fragment thereof comprising a heavy chain and a light chain, and wherein
the second antigen-binding site is comprised by an anti-FX(a) antibody or antigen-binding fragment thereof comprising a heavy chain and a light chain,
wherein
a. the anti-FIX(a) antibody heavy chain CDR1-3 sequences are identified by SEQ ID NOs:36, 37 and 38, respectively, optionally comprising 1, 2 or 3 amino acid substitutions, and
the anti-FIX(a) antibody light chain CDR1-3 sequences are identified by SEQ ID NOs:40, 41 and 42, respectively, optionally comprising 1, 2 or 3 amino acid substitutions, and
the anti-FX(a) antibody heavy chain CDR1-3 sequences are identified by SEQ ID NOs:468, 469 and 470, respectively, optionally comprising 1, 2 or 3 amino acid substitutions, and
the anti-FX(a) antibody light chain CDR1-3 sequences are identified by SEQ ID NOs:472, 473 and 474, respectively, optionally comprising 1, 2 or 3 amino acid substitutions, or
b. the anti-FIX(a) antibody heavy chain CDR1-3 sequences are identified by SEQ ID NOs:36, 37 and 38, respectively, optionally comprising 1, 2 or 3 amino acid substitutions, and
the anti-FIX(a) antibody light chain CDR1-3 sequences are identified by SEQ ID NOs:40, 41 and 42, respectively, optionally comprising 1, 2 or 3 amino acid substitutions, and
the anti-FX(a) antibody heavy chain CDR1-3 sequences are identified by SEQ ID NOs:484, 485 and 486, respectively, optionally comprising 1, 2 or 3 amino acid substitutions, and
the anti-FX(a) antibody light chain CDR1-3 sequences are identified by SEQ ID NOs:488, 489 and 490, respectively, optionally comprising 1, 2 or 3 amino acid substitutions, or
c. the anti-FIX(a) antibody heavy chain CDR1-3 sequences are identified by SEQ ID NOs:44, 45 and 46, respectively, optionally comprising 1, 2 or 3 amino acid substitutions, and
the anti-FIX(a) antibody light chain CDR1-3 sequences are identified by SEQ ID NOs:48, 49 and 50, respectively, optionally comprising 1, 2 or 3 amino acid substitutions, and
the anti-FX(a) antibody heavy chain CDR1-3 sequences are identified by SEQ ID NOs: 468, 469 and 470, respectively, optionally comprising 1, 2 or 3 amino acid substitutions, and
the anti-FX(a) antibody light chain CDR1-3 sequences are identified by SEQ ID NOs: 472, 473 and 474, respectively, optionally comprising 1, 2 or 3 amino acid substitutions, or
d. the anti-FIX(a) antibody heavy chain CDR1-3 sequences are identified by SEQ ID NOs:52, 53 and 54, respectively, optionally comprising 1, 2 or 3 amino acid substitutions, and
the anti-FIX(a) antibody light chain CDR1-3 sequences are identified by SEQ ID NOs: 56, 57 and 58, respectively, optionally comprising 1, 2 or 3 amino acid substitutions, and
the anti-FX(a) antibody heavy chain CDR1-3 sequences are identified by SEQ ID NOs:468, 469 and 470, respectively, optionally comprising 1, 2 or 3 amino acid substitutions, and
the anti-FX(a) antibody light chain CDR1-3 sequences are identified by SEQ ID NOs:472, 473 and 474, respectively, optionally comprising 1, 2 or 3 amino acid substitutions, or
e. the anti-FIX(a) antibody heavy chain CDR1-3 sequences are identified by SEQ ID NOs: 52, 53 and 54, respectively, optionally comprising 1, 2 or 3 amino acid substitutions, and
the anti-FIX(a) antibody light chain CDR1-3 sequences are identified by SEQ ID NOs:56, 57 and 58, respectively, optionally comprising 1, 2 or 3 amino acid substitutions, and
the anti-FX(a) antibody heavy chain CDR1-3 sequences are identified by SEQ ID NOs: 484, 485 and 486, respectively, optionally comprising 1, 2 or 3 amino acid substitutions, and
the anti-FX(a) antibody light chain CDR1-3 sequences are identified by SEQ ID NOs:488, 489 and 490, respectively, optionally comprising 1, 2 or 3 amino acid substitutions, or
f. the anti-FIX(a) antibody heavy chain CDR1-3 sequences are identified by SEQ ID NOs:68, 69 and 70, respectively, optionally comprising 1, 2 or 3 amino acid substitutions, and
the anti-FIX(a) antibody light chain CDR1-3 sequences are identified by SEQ ID NOs:72, 73 and 74, respectively, optionally comprising 1, 2 or 3 amino acid substitutions, and
the anti-FX(a) antibody heavy chain CDR1-3 sequences are identified by SEQ ID NOs: 468, 469 and 470, respectively, optionally comprising 1, 2 or 3 amino acid substitutions, and
the anti-FX(a) antibody light chain CDR1-3 sequences are identified by SEQ ID NOs:472, 473 and 474, respectively, optionally comprising 1, 2 or 3 amino acid substitutions, or
g. the anti-FIX(a) antibody heavy chain CDR1-3 sequences are identified by SEQ ID NOs:68, 69 and 70, respectively, optionally comprising 1, 2 or 3 amino acid substitutions, and
the anti-FIX(a) antibody light chain CDR1-3 sequences are identified by SEQ ID NOs:72, 73 and 74, respectively, optionally comprising 1, 2 or 3 amino acid substitutions, and
the anti-FX(a) antibody heavy chain CDR1-3 sequences are identified by SEQ ID NOs:484, 485 and 486, respectively, optionally comprising 1, 2 or 3 amino acid substitutions, and
the anti-FX(a) antibody light chain CDR1-3 sequences are identified by SEQ ID NOs: 488, 489 and 490, respectively, optionally comprising 1, 2 or 3 amino acid substitutions, or
h. the anti-FIX(a) antibody heavy chain CDR1-3 sequences are identified by SEQ ID NOs:1203, 1204 and 1205, respectively, optionally comprising 1, 2 or 3 amino acid substitutions, and
the anti-FIX(a) antibody light chain CDR1-3 sequences are identified by SEQ ID NOs:1207, 1208 and 1209, respectively, optionally comprising 1, 2 or 3 amino acid substitutions, and
the anti-FX(a) antibody heavy chain CDR1-3 sequences are identified by SEQ ID NOs:468, 469 and 470, respectively, optionally comprising 1, 2 or 3 amino acid substitutions, and
the anti-FX(a) antibody light chain CDR1-3 sequences are identified by SEQ ID NOs:472, 473, and 474, respectively, optionally comprising 1, 2 or 3 amino acid substitutions, or
i. the anti-FIX(a) antibody heavy chain CDR1-3 sequences are identified by SEQ ID NOs:1211, 1212 and 1213, respectively, optionally comprising 1, 2 or 3 amino acid substitutions, and
the anti-FIX(a) antibody light chain CDR1-3 sequences are identified by SEQ ID NOs:1215, 1216 and 1217, respectively, optionally comprising 1, 2 or 3 amino acid substitutions, and
the anti-FX(a) antibody heavy chain CDR1-3 sequences are identified by SEQ ID NOs:468, 469 and 470, respectively, optionally comprising 1, 2 or 3 amino acid substitutions, and
the anti-FX(a) antibody light chain CDR1-3 sequences are identified by SEQ ID NOs:472, 473, and 474, respectively, optionally comprising 1, 2 or 3 amino acid substitutions, or
j. the anti-FIX(a) antibody heavy chain CDR1-3 sequences are identified by SEQ ID NOs:1219, 1220 and 1221, respectively, optionally comprising 1, 2 or 3 amino acid substitutions, and
the anti-FIX(a) antibody light chain CDR1-3 sequences are identified by SEQ ID NOs:1223, 1224 and 1225, respectively, optionally comprising 1, 2 or 3 amino acid substitutions, and
the anti-FX(a) antibody heavy chain CDR1-3 sequences are identified by SEQ ID NOs:468, 469 and 470, respectively, optionally comprising 1, 2 or 3 amino acid substitutions, and
the anti-FX(a) antibody light chain CDR1-3 sequences are identified by SEQ ID NOs:472, 473, and 474, respectively, optionally comprising 1, 2 or 3 amino acid substitutions, or
k. the anti-FIX(a) antibody heavy chain CDR1-3 sequences are identified by SEQ ID NOs:1227, 1228 and 1229, respectively, optionally comprising 1, 2 or 3 amino acid substitutions, and
the anti-FIX(a) antibody light chain CDR1-3 sequences are identified by SEQ ID NOs:1231, 1232 and 1233, respectively, optionally comprising 1, 2 or 3 amino acid substitutions, and
the anti-FX(a) antibody heavy chain CDR1-3 sequences are identified by SEQ ID NOs:468, 469 and 470, respectively, optionally comprising 1, 2 or 3 amino acid substitutions, and
the anti-FX(a) antibody light chain CDR1-3 sequences are identified by SEQ ID NOs:472, 473, and 474, respectively, optionally comprising 1, 2 or 3 amino acid substitutions, or
l. the anti-FIX(a) antibody heavy chain CDR1-3 sequences are identified by SEQ ID NOs:1235, 1236 and 1337, respectively, optionally comprising 1, 2 or 3 amino acid substitutions, and
the anti-FIX(a) antibody light chain CDR1-3 sequences are identified by SEQ ID NOs:1239, 1240 and 1241, respectively, optionally comprising 1, 2 or 3 amino acid substitutions, and
the anti-FX(a) antibody heavy chain CDR1-3 sequences are identified by SEQ ID NOs:468, 469 and 470, respectively, optionally comprising 1, 2 or 3 amino acid substitutions, and
the anti-FX(a) antibody light chain CDR1-3 sequences are identified by SEQ ID NOs:472, 473, and 474, respectively, optionally comprising 1, 2 or 3 amino acid substitutions, or
m. the anti-FIX(a) antibody heavy chain CDR1-3 sequences are identified by SEQ ID NOs:1243, 1244 and 1245, respectively, optionally comprising 1, 2 or 3 amino acid substitutions, and
the anti-FIX(a) antibody light chain CDR1-3 sequences are identified by SEQ ID NOs:1247, 1248 and 1249, respectively, optionally comprising 1, 2 or 3 amino acid substitutions, and
the anti-FX(a) antibody heavy chain CDR1-3 sequences are identified by SEQ ID NOs:468, 469 and 470, respectively, optionally comprising 1, 2 or 3 amino acid substitutions, and
the anti-FX(a) antibody light chain CDR1-3 sequences are identified by SEQ ID NOs:472, 473, and 474, respectively, optionally comprising 1, 2 or 3 amino acid substitutions.

13. The antibody or antigen-binding fragment thereof according to claim 1, wherein
the first antigen-binding site is comprised by an anti-FIX(a) antibody or antigen-binding fragment thereof comprising a heavy chain and a light chain, and
the second antigen-binding site is comprised by an anti-FX(a) antibody or antigen-binding fragment thereof comprising a heavy chain and a light chain,
wherein the anti-FIX(a) antibody or antigen-binding fragment thereof comprises
a. a heavy chain variable domain identified by SEQ ID NO:35 and a light chain variable domain identified by SEQ ID NO:39, or
b. a heavy chain variable domain identified by SEQ ID NO:43 and a light chain variable domain identified by SEQ ID NO:47, or
c. a heavy chain variable domain identified by SEQ ID NO:51 and a light chain variable domain identified by SEQ ID NO:55, or
d. a heavy chain variable domain identified by SEQ ID NO:67 and a light chain variable domain identified by SEQ ID NO:71,
e. a heavy chain variable domain identified by SEQ ID NO:1202 and a light chain variable domain identified by SEQ ID NO:1206,
f. a heavy chain variable domain identified by SEQ ID NO:1210 and a light chain variable domain identified by SEQ ID NO:1214,
g. a heavy chain variable domain identified by SEQ ID NO:1218 and a light chain variable domain identified by SEQ ID NO:1222,
h. a heavy chain variable domain identified by SEQ ID NO:1226 and a light chain variable domain identified by SEQ ID NO:1230,
i. a heavy chain variable domain identified by SEQ ID NO:1234 and a light chain variable domain identified by SEQ ID NO:1238, or
j. a heavy chain variable domain identified by SEQ ID NO:1242 and a light chain variable domain identified by SEQ ID NO:1246,
and wherein the anti-FX(a) antibody or antigen-binding fragment thereof comprises
k. a heavy chain variable domain identified by SEQ ID NO:467 and a light chain variable domain identified by SEQ ID NO:471, or
l. a heavy chain variable domain identified by SEQ ID NO:483 and a light chain variable domain identified by SEQ ID NO:487.

14. The antibody or antigen-binding fragment thereof according to claim 13, wherein both the heavy chain variable domains and the light chain variable domains are at least 96, 97, 98, 99, 99.1 or 99.2% identical to the recited SEQ ID NOs.

15. The antibody or antigen-binding fragment thereof according to claim 1, wherein the antibody or antigen-binding fragment thereof is a bispecific antibody.

16. A method of treating haemophilia A with or without inhibitors comprising administering to a subject in need thereof the antibody or antigen-binding fragment thereof according to claim 1.

17. A pharmaceutical composition comprising the antibody or antigen-binding fragment thereof according to claim 1 and one or more pharmaceutically acceptable carrier(s).

18. An antibody or antigen-binding fragment thereof which is capable of binding to FIX (SEQ ID NO:1) and/or the activated form thereof (FIXa) and capable of stimulating the enzymatic activity of FIXa towards FX comprising a heavy chain and a light chain wherein
a. the heavy chain CDR1-3 sequences are identified by SEQ ID NOs:36, 37 and 38, respectively, optionally comprising 1, 2 or 3 amino acid substitutions, and
the light chain CDR1-3 sequences are identified by SEQ ID NOs:40, 41 and 42, respectively, optionally comprising 1, 2 or 3 amino acid substitutions, or
b. the heavy chain CDR1-3 sequences are identified by SEQ ID NOs:44, 45 and 46, respectively, optionally comprising 1, 2 or 3 amino acid substitutions, and
the light chain CDR1-3 sequences are identified by SEQ ID NOs:48, 49 and 50, respectively, optionally comprising 1, 2 or 3 amino acid substitutions, or
c. the heavy chain CDR1-3 sequences are identified by SEQ ID NOs:52, 53 and 54, respectively, optionally comprising 1, 2 or 3 amino acid substitutions, and
the light chain CDR1-3 sequences are identified by SEQ ID NOs: 56, 57 and 58, respectively, optionally comprising 1, 2 or 3 amino acid substitutions, or
d. the heavy chain CDR1-3 sequences are identified by SEQ ID NOs:68, 69 and 70, respectively, optionally comprising 1, 2 or 3 amino acid substitutions, and
the light chain CDR1-3 sequences are identified by SEQ ID NOs:72, 73 and 74, respectively, optionally comprising 1, 2 or 3 amino acid substitutions, or
e. the heavy chain CDR1-3 sequences are identified by SEQ ID NOs:1203, 1204 and 1205, respectively, optionally comprising 1, 2 or 3 amino acid substitutions, and
the light chain CDR1-3 sequences are identified by SEQ ID NOs:1207, 1208 and 1209, respectively, optionally comprising 1, 2 or 3 amino acid substitutions, or
f. the heavy chain CDR1-3 sequences are identified by SEQ ID NOs:1211, 1212 and 1213, respectively, optionally comprising 1, 2 or 3 amino acid substitutions, and
the light chain CDR1-3 sequences are identified by SEQ ID NOs:1215, 1216 and 1217, respectively, optionally comprising 1, 2 or 3 amino acid substitutions, or
g. the heavy chain CDR1-3 sequences are identified by SEQ ID NOs:1219, 1220 and 1221, respectively, optionally comprising 1, 2 or 3 amino acid substitutions, and
the light chain CDR1-3 sequences are identified by SEQ ID NOs:1223, 1224 and 1225, respectively, optionally comprising 1, 2 or 3 amino acid substitutions, or
h. the heavy chain CDR1-3 sequences are identified by SEQ ID NOs:1227, 1228 and 1229, respectively, optionally comprising 1, 2 or 3 amino acid substitutions, and the light chain CDR1-3 sequences are identified by SEQ ID NOs:1231, 1232 and 1233, respectively, optionally comprising 1, 2 or 3 amino acid substitutions, or
i. the heavy chain CDR1-3 sequences are identified by SEQ ID NOs:1235, 1236 and 1337, respectively, optionally comprising 1, 2 or 3 amino acid substitutions, and
the light chain CDR1-3 sequences are identified by SEQ ID NOs:1239, 1240 and 1241, respectively, optionally comprising 1, 2 or 3 amino acid substitutions, or
j. the heavy chain CDR1-3 sequences are identified by SEQ ID NOs:1243, 1244 and 1245, respectively, optionally comprising 1, 2 or 3 amino acid substitutions, and
the light chain CDR1-3 sequences are identified by SEQ ID NOs:1247, 1248 and 1249, respectively, optionally comprising 1, 2 or 3 amino acid substitutions;
wherein said antibody or antigen-binding fragment thereof is an intermediate for use in the manufacture of a multispecific antibody which is capable of binding to FIX (SEQ ID NO:1) and/or the activated form thereof (FIXa), and capable of binding to FX (SEQ ID NO:2) and/or the activated form thereof (FXa).

19. A method of treating haemophilia A with or without inhibitors comprising administering to a subject in need thereof the antibody or antigen-binding fragment thereof according to claim 2.

20. A pharmaceutical composition comprising the antibody or antigen-binding fragment thereof according to claim 2 and one or more pharmaceutically acceptable carrier(s).
